(19) United States
(12) Patent Application Publication
(10) Pub. No.: US 2023/0391772 A1
(43) Pub. Date: Dec. 7, 2023

(54) METHODS AND COMPOSITIONS FOR TARGETED PROTEIN DEGRADATION

(71) Applicant: Ranok Therapeutics (Hangzhou) Co. Ltd., Hangzhou (CN)

(72) Inventors: Weiwen Ying, Lexington, MA (US); Kevin P. Foley, Waltham, MA (US); Mingkai Wang, Hangzhou (CN); Chenghao Ying, Shanghai (CN); Long Ye, Shanghai (CN); Wei Yin, Lianyungang (CN); Lingjie Zhang, Wuxi (CN)

(21) Appl. No.: 18/031,679

(22) PCT Filed: Oct. 13, 2021
(86) PCT No.: PCT/CN2021/123660

§ 371 Date: Apr. 13, 2023

(30) Foreign Application Priority Data
Oct. 14, 2020 (WO) PCT/CN2020/120927

(51) Int. Cl. C07D 471/04; C07D 519/00; A61P 35/00

(52) U.S. Cl. CPC C07D 471/04; C07D 519/00; A61P 35/00

(57) ABSTRACT

Provided are compounds of Formula (I) and pharmaceutically acceptable salts and compositions thereof, which are useful for treating cancers and related conditions.

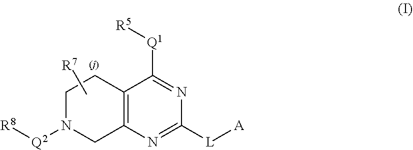

### RELATED APPLICATIONS

This application claims priority to PCT/CN2020/120927, filed Oct. 14, 2020, the entire contents of which are incorporated herein by reference.

### BACKGROUND

Protein homeostasis, or proteostasis, refers to the ability of cells to regulate the synthesis, folding, trafficking and degradation of proteins. In particular, properly regulated protein degradation is required for the normal functioning of cells, including their proliferation, differentiation and death, and is often dysregulated in cancers and other diseases (Van Die, Chin J Cancer, 2011, 30:124-137).

The ubiquitin-proteasome system (UPS) is one of the major pathways in cells that mediates the disposal and metabolic recycling of proteins (Yu and Matouschek, Annu Rev Biophys, 2017, 46:149-173; Navon and Ciechanover, J Biol Chem, 2009, 284:33713-33718). Ubiquitin is a 76 amino acid-residue protein that is ubiquitously expressed. With respect to protein degradation by the UPS, the process of ubiquitination occurs when a ubiquitin is attached to a lysine amino acid residue in a substrate protein, which involves a series of enzymatic steps. First, ubiquitin is transferred to an E1 ubiquitin-activating enzyme. Second, activated ubiquitin is transferred from the E1 to an E2 ubiquitin-conjugating enzyme. And third, one of the several hundred different E3 ubiquitin ligase enzymes links the ubiquitin to a lysine residue in a substrate protein. Repetition of this enzymatic process results in tagging substrate proteins with polyubiquitin chains. Such ubiquitin-tagged proteins can then be delivered to the proteasome, a large multi-subunit complex that degrades proteins. The ability of some cellular chaperone proteins and chaperone complexes to direct proteins towards the UPS is facilitated by their direct interaction with E3 ubiquitin ligases (Amm et al., Biochim Biophys Acta, 2014, 1843:182-196; Taipale et al., Cell, 2012, 150:987-1001). In addition to protein degradation, the ubiquitination of proteins can also regulate other processes, such as subcellular localization, activity and protein-protein interactions.

Chemically induced, targeted protein degradation (TPD) has emerged as a new modality for small molecule drug development. A small molecule can be used to promote the interaction of a target protein or proteins with a component or components of various cellular protein degradation pathways, thereby inducing the degradation of the targeted protein or proteins as a way to treat disease.

In particular, proteolysis-targeting chimeras (PROTACs) are an example of such small molecules that purposely induce protein degradation of specific proteins by coopting the UPS (Burslem and Crews, Cell, 2020, 181:102-114; Pettersson and Crews, Drug Discov Today Technol, 2019, 31:15-27). PROTAC molecules are bifunctional small molecules that simultaneously bind to a target protein or proteins and an E3 ubiquitin ligase, creating ternary complexes in cells between the target protein(s), the PROTAC molecule and an E3 ligase protein. The induced proximity of the target protein(s) and the E3 ligase causes the ubiquitination of the target protein(s) and subsequent degradation of the target protein(s) by the proteasome. Although PROTACs that incorporate target protein binders that promiscuously bind to multiple proteins can often degrade multiple proteins, in some cases protein-protein interactions between individual targets and an E3 ligase can increase or decrease the observed potency and selectivity of degradation, for example by inhibiting formation of some ternary complexes due to charge repulsion and steric clashing between a given target protein and E3 ligase pair (Pettersson and Crews, Drug Discov Today Technol, 2019, 31:15-27; Bondeson et al., Cell Chem Biol, 2018, 25:78-87; Gadd et al., Nat Chem Biol, 2017, 13:514-521; Zengerle et al., ACS Chem Biol, 2015, 10:1770-1777).

Other methods to chemically induce TPD have also been described, such as molecular glues (Che et al., Bioog Med Chem Lett, 2018, 28:2585-2592), AUTACs, ATTECs and LYTACs (Ding et al., Trends Pharmacol Sci, 2020, 41:464-474). For example, AUTAC technology follows a similar principle of induced proximity, but targets proteins for degradation via autophagy (Daiki et al., Mol Cell, 2019, 76:797-810).

Collectively, TPD technologies have a number of advantages over conventional biochemical inhibitors (Pettersson and Crews, Drug Discov Today Technol, 2019, 31:15-27; Ding et al., Trends Pharmacol Sci, 2020, 41:464-474). For example, unlike conventional inhibitors, TPD agents work sub-stoichiometrically and can typically mediate the sequential degradation of multiple molecules of the target protein(s), often leading to greater potency than the isolated target binding moiety that they incorporate and other biochemical inhibitors. Also, since inhibition of target protein(s) function by TPD agents is principally due to degradation rather than solely biochemical inhibition, recovery of the function of target protein(s) is typically slower than is observed for biochemical inhibitors. TPD agents may also have improved target selectivity over biochemical inhibitors. Finally, TPD agents can target proteins that are not amenable to biochemical inhibition by interacting with binding pockets that do not affect the biochemical activity of the target but still permit its degradation.

However, some disadvantages are associated with current TPD technologies. These include the promiscuous degradation of the target protein(s) in many tissues and organs, not just the tissue(s) and organ(s) where the target protein(s) is involved in a disease process, which is expected to result in unwanted side effects of treatment. Also, resistance to these technologies can develop through mutations or alterations in expression of components of the UPS such as E3 ligases (Ottis et al., ACS Chem Biol, 2019, 14:2215-2223; Zhang et al., Mol Cancer Ther, 2019, 18:1302-1311), resulting in loss of therapeutic efficacy. As such, a need exists for improved/alternative methods and compositions for TPD.

It is also desirable to develop improved/alternative TPD agents that mediate the degradation of proteins involved in cancer and other diseases. Activating mutations in KRAS are among the most frequent oncogenic changes found in human cancers (Simanshu et al., Cell, 2017, 170:17-33). In particular, mutated KRAS(G12C) is found in ˜15% of lung adenocarcinomas, ˜8% of colorectal carcinomas and ˜4% of pancreatic adenocarcinomas, as well as in other cancers (Jiao and Yang, Innovation (N Y), 2020, 1:100035). Although mutated KRAS has historically been considered an undruggable cancer drug target, recent progress has led to the development of covalently binding pharmacological inhibitors of KRAS(G12C) (Ostrem et al., Nature, 2013, 503:548-551; Cannon et al., Nature, 2019, 575:217-223; O'Bryan, Pharmacol Res, 2019, 139:503-511; Hallin et al., Cancer Discov, 2020, 10:54-71). Although efficacy has been observed for such inhibitors in cancer patients in the clinic, innate resistance and tumor relapses have also been reported (Cannon et al., Nature, 2019, 575:217-223). The mechanisms underlying the innate and acquired resistance to covalent KRAS(G12C) inhibitors have also begun to be elucidated, mediated for example, through high EGFR signaling (Adachi et al., Clin Cancer Res, 2020, in press; Amodio et al., Cancer Discov, 2020; Hata and Shaw, Nat Med, 2020, 26:169-170; Jiao and Yang, Innovation (N Y), 2020, 1:100035; Koleilat and Kwong, Cancer Discov, 2020, 10:1094-1096; Ryan et al., Clin Cancer Res, 2020, 26:1633-1643; Xue et al., Nature, 2020, 577:421-425). As such, KRAS(G12C) represents an attractive drug target and it is desirable to develop agents that induce TPD of KRAS(G12C). It is also desirable to develop agents that induce degradation of both KRAS(G12C) and one or more other proteins, particularly those involved in mediating resistance to KRAS(G12C) inhibitors.

### SUMMARY

The present disclosure provides tumor-targeted protein degradation chimeras, termed chaperone-mediated protein degraders (CHAMPs) comprising a first moiety that is capable of binding to a target protein (e.g., KRAS(G12C)) or proteins and a second moiety that is capable of binding a chaperone protein or proteins or protein component of chaperone complexes (e.g., HSP90). Such CHAMP compounds include those having the Formula I

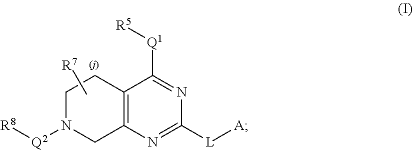

and pharmaceutically acceptable salts thereof, wherein L, A, Q¹, Q², R⁵, R⁷, R⁸, and j are as defined herein.

Compositions comprising the disclosed compounds of Formula I as well as methods for their manufacture are also provided. In one aspect, the disclosed compounds induce targeted oncogenic protein degradation in a tumor-selective fashion and are useful in the treatment of cancer and related conditions.

### DETAILED DESCRIPTION

### 1. General Description of Compounds

Provided herein are CHAMP compounds having the Formula I

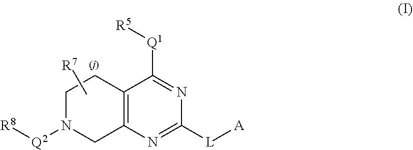

or a pharmaceutically acceptable salt thereof, wherein,
A is a chemical moiety that binds HSP90 protein;
L is a linker;
Q¹ is a nitrogen containing heteroaryl or heterocyclyl ring, each of which are optionally substituted with 1 to 3 groups selected from R⁶;
R⁵ is —C(O)Y or —S(O)₂Y;
Y is a (C₁-C₆)alkyl, halo(C₁-C₆)alkyl, (C₂-C₆)alkenyl, halo(C₂-C₆)alkenyl, NH₂, —NH(C₁-C₆)alkyl, —N[(C₁-C₆)alkyl]₂, NHNH₂, or NHOH, wherein said (C₂-C₆)alkenyl, alone or as recited in halo(C₂-C₆)alkenyl, is optionally substituted with (C₁-C₆)alkyl, halo(C₁—C₆)alkyl, heteroalkyl, hydroxy(C₁-C₆)alkyl, —C(O)NH₂, —C(O)NH(C₁-C₆)alkyl, or —C(O)N[(C₁-C₆)alkyl]₂;
R⁶ is (C₁-C₆)alkyl, halo(C₁-C₆)alkyl, (C₁-C₆)alkoxy, halo(C₁-C₆)alkoxy, hydroxy(C₁-C₆)alkyl, cyano(C₁-C₆)alkyl, oxo, cyano, heteroalkyl, —C(O)OH, —C(O)O(C₁-C₆)alkyl, —C(O)NH₂, —C(O)NH(C₁-C₆)alkyl, or —C(O)N[(C₁-C₆)alkyl]₂, wherein said (C₁-C₆)alkyl is optionally substituted with heteroaryl;
R⁷ is halo, hydroxyl, (C₁-C₆)alkyl, halo(C₁-C₆)alkyl, (C₁-C₆)alkoxy, halo(C₁-C₆)alkoxy, cycloalkyl, heteroalkyl, hydroxy(C₁-C₆)alkyl, or S(C₁-C₆)alkyl;
j is 1 or 2;
Q² is a bond, —C(O)—, or (C₁-C₃)alkylene;
R⁸ is cycloalkyl, heterocyclyl, aryl, or heteroaryl, each of which are optionally substituted with 1 to 3 groups selected from R⁹;
R⁹ is halo, (C₁-C₆)alkyl, (C₂-C₆)alkenyl, halo(C₂-C₆)alkenyl, (C₂-C₆)alkynyl, oxo, cyano, —(C₁-C₆)alkylOR^{c}, —(C₁-C₆)alkylN(R^{d})₂, —(C₁-C₆)alkylC(O)OR^{d}, OH, —(C₁-C₆)alkylC(O)N(R^{d})₂, —(C₁-C₆)alkylO(C₁-C₆)alkylN(R^{d})₂, —(C₁-C₆)alkylSOR^{d}, —(C₁-C₆)alkylS(O)₂R^{d}, —(C₁-C₆)alkylSON(R^{d})₂, —(C₁-C₆)alkylSO₂N(R^{d})₂, —(C₁-C₆)alkylcycloalkyl, —(C₁-C₆)alkylheterocyclyl, —(C₁-C₆)alkylheteroaryl, —(C₁-C₆)alkylaryl, —(C₁-C₆)alkoxy, halo(C₁-C₆)alkoxy, CN, aryl, heteroaryl, cycloalkyl, heterocycloalkyl, —C(O)R^{d}, —C(O)OR^{d}, —C(O)N(R^{d})₂, N(R^{d})₂, —C(O)NR^{d}(C₁-C₆)alkylN(R^{d})₂, —NR^{d}(C₁-C₆)alkylN(R^{d})₂, —NR^{d}(C₁-C₆)alkylOR^{d}, —SOR^{d}, —S(O)₂R^{d}, —SON(R^{d})₂, —SO₂N(R^{d})₂, or CN, wherein each aryl, cycloalkyl, heterocyclyl, and heteoaryl alone and in connection with —(C₁-C₆)alkylcycloalkyl, —(C₁-C₆)alkylheterocyclyl, —(C₁-C₆)alkylheteroaryl, —(C₁-C₆)alkylaryl are optionally substituted with 1 to 3 groups selected from R^{e};
R^{c} and R^{d} are each independently selected from hydrogen, (C₁-C₆)alkyl, and halo(C₁-C₆)alkyl; and
R^{e} is selected from halo, oxo, CN, NO₂, —N(R^{d})₂, —OR^{d}, —C(O)OR^{d}, (C₁-C₆)alkyl, —(C₁-C₆)alkylOR^{c}, halo(C₁-C₆)alkyl, (C₁-C₆)alkoxy, halo(C₁-C₆)alkoxy, —(C₁-C₆)alkylC(O)OR^{d}, —(C₁-C₆)alkylC(O)N(R^{d})₂, (C₂-C₆)alkenyl, halo(C₂-C₆)alkenyl, (C₂-C₆)alkynyl, —(C₁-C₆)alkylSR^{d}, —(C₁-C₆)alkylOR^{c}, —(C₁-C₆)alkylN(R^{d})₂, —C(O)N(R^{d})₂, —C(O)NRdC₁₋₆alkylN(R^{d})₂, —NR^{d}C₁₋₆alkylN(R^{d})₂, —NR^{d}C₁₋₆alkylOR^{d}, —SOR^{d}, —S(O)₂R^{d}, —SON(R^{d})₂, —SO₂N(R^{d})₂, aryl, heteroaryl, cycloalkyl, and heterocycloalkyl.

### 2. Definitions

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” are used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, the term “alkyl” means a saturated straight chain or branched non-cyclic hydrocarbon having, unless specified otherwise, from 1 to 10 carbon atom e.g., (C₁-C₆)alkyl or (C₁-C₄)alkyl. Representative straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

As used herein, the term “alkenyl” means a saturated straight chain or branched non-cyclic hydrocarbon having, unless specified otherwise, from 2 to 10 carbon atoms (e.g., (C₂-C₆)alkenyl or (C₂-C₄)alkenyl) and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like.

As used herein, the term “alkynyl” means a saturated straight chain or branched non-cyclic hydrocarbon having, unless specified otherwise, from 2 to 10 carbon atoms (e.g., (C₂-C₆)alkynyl or (C₂-C₄)alkynyl) and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like.

As used herein, the term “cycloalkyl” means a saturated, monocyclic alkyl radical having from e.g., 3 to 10 carbon atoms (e.g., from 4 to 6 carbon atoms). Representative cycloalkyls include cyclopropyl, cyclobutyl, cyclopentyl, cyclohexyl, cycloheptyl, cyclooctyl, cyclononyl, and cyclodecanyl.

The term “oxo” refers to the group ═O.

As used herein, the term “haloalkyl” means and alkyl group in which one or more (including all) the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker.

As used herein, an “haloalkoxy” is an haloalkyl group which is attached to another moiety via an oxygen linker.

As used herein, the term “alkylene” refers to an alkyl group that has two points of attachment. Straight chain alkylene groups are preferred. Non-limiting examples of alkylene groups include methylene ethylene, n-propylene, isopropylene, and the like. Alkylene groups may be optionally substituted with one or more substituents.

As used herein, the term “heterocyclyl” means a monocyclic heterocyclic ring system which is either a saturated ring or an unsaturated non-aromatic ring comprising, as size and valency permits, up to 5 heteroatoms independently selected from nitrogen, oxygen, and sulfur. The heterocycle may be attached via any heteroatom or carbon atom. Representative heterocycles include morpholinyl, thiomorpholinyl, pyrrolidinonyl, pyrrolidinyl, piperidinyl, piperazinyl, oxiranyl, oxetanyl, tetrahydrofuranyl, tetrahydropyranyl, tetrahydropyrindinyl, tetrahydropyrimidinyl, and the like.

As used herein, the term “heteroaryl” means, as the defined size permits, a monocyclic or polycyclic heteroaromatic ring comprising carbon atom ring members and one or more heteroatom ring members selected from nitrogen, oxygen, and sulfur. Representative heteroaryl groups include pyridyl, furanyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, benzothienyl, and the like. The point of attachment of a heteroaromatic or heteroaryl ring to another group may be at either a carbon atom or a heteroatom of the heteroaromatic or heteroaryl rings.

As used herein, the term “halogen” or “halo” means F, Cl, Br or I.

When a heterocyclyl or heteroaryl, group contains a nitrogen atom, it may be substituted or unsubstituted as valency permits.

The term “linker” or “tether,” used interchangeably, refers to a chemical moiety that joins two other moieties (e.g., a first binding moiety and a second binding moiety). A linker can covalently join a first binding moiety and a second binding moiety. In one aspect, the linker is uncleavable in vivo. In one aspect, the linker comprises one or more cyclic ring systems. In another aspect, the linker comprises an alkyl chain optionally substituted by and/or interrupted with one or more chemical groups. In one aspect, the linker comprises optimal spatial and chemical properties to effectuate optimal therapeutic activity. In one aspect, the linker does not interfere with the ability of the first binding moiety and/or the second binding moiety to bind their respective targets (e.g., HSP90 and KRAS(G12C)). In one aspect, the linker alters the ability of the first binding moiety and/or the second binding moiety to bind their respective targets (e.g., HSP90 and KRAS(G12C)).

The term “KRAS” refers to the protein product of the KRAS proto-oncogene, GTPase gene.

The term “KRAS(G12C)” refers to the protein product of the KRAS gene carrying a mutation that results in the glycine amino acid at position 12 of KRAS being replaced by a cysteine.

The term “HSP90” refers collectively, individually or in various combinations to the protein products of members of the heat shock protein 90 (90 kDa) gene family, including: HSP90AA1 (HSP90-alpha or HSP90a), HSP90AB1 (HSP90-beta or HSP900), HSP90B1 (GRP94) and TRAP1.

When used in connection to describe a chemical group that may have multiple points of attachment, a hyphen (-) designates the point of attachment of that group to the variable to which it is defined. For example, —NR^{a}R^{b} and —C(O)NR^{a}(C₁₋₄alkylene)NR^{a}R mean that the point of attachment for these groups occur on the nitrogen atom and carbon atom respectively.

A hash bond as in “ [symbol] ” represents the point at which the depicted group is attached to the defined variable.

When the stereochemistry of a disclosed compound is named or depicted by structure, the named or depicted stereoisomer is at least 60%, 70%, 80%, 90%, 99% or 99.9% by weight pure relative to all of the other stereoisomers. Percent by weight pure relative to all of the other stereoisomers is the ratio of the weight of one stereoisomer over the weight of the other stereoisomers. For example, when a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

For use in medicines, the pharmaceutically acceptable salts of the disclosed compounds refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) of the state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations. In one aspect, a therapeutically effective amount refers to a dosage of from about 0.01 to about 100 mg/kg body weight/day.

The terms “administer,” “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. In another preferred embodiment, an agent is administered orally. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### 3. Compounds

In a first embodiment, provided is a compound of the Formula I

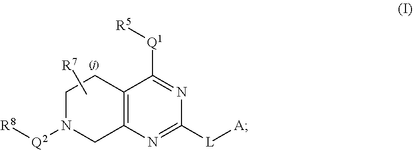

or a pharmaceutically acceptable salt thereof, wherein the variables are as described above.

In a second embodiment, A in the compound of Formula I is selected from

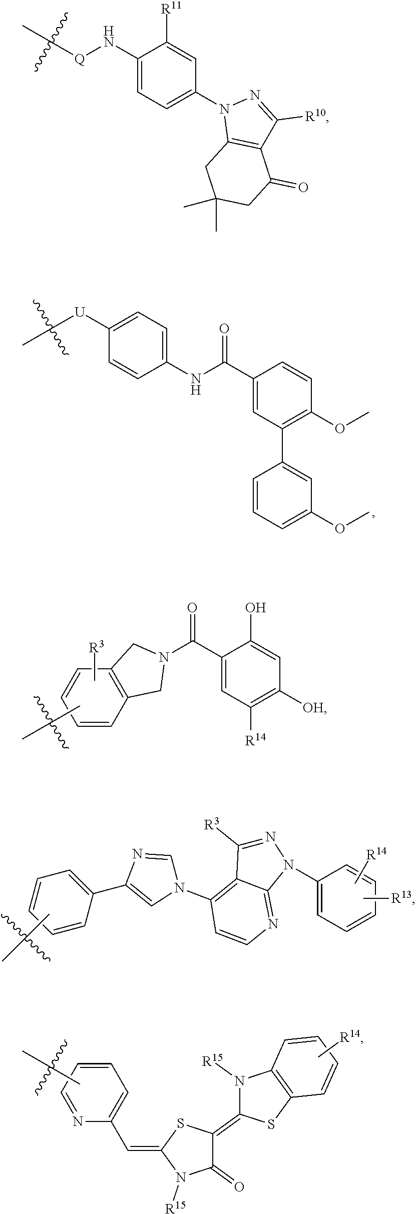

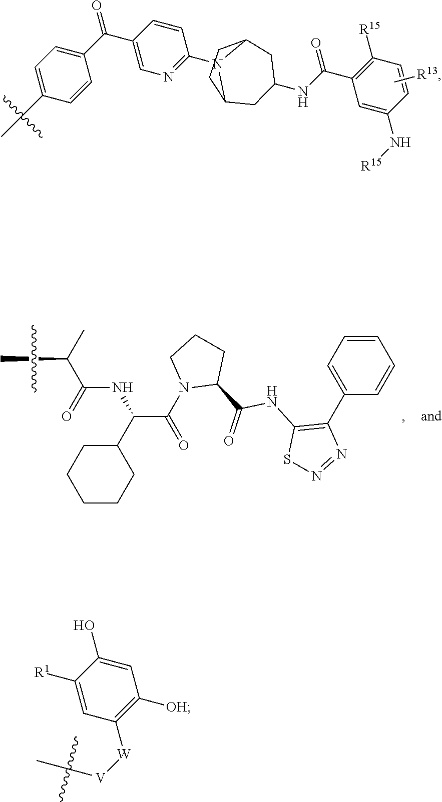

wherein
Q and U are each independently selected from phenyl, heteroaryl, heterocyclyl, and cycloalkyl, each of which being optionally substituted with 1 to 3 groups selected from R²
   R¹³ and R¹⁴ are each independently selected from hydrogen, halo, —CN, (C₁-C₄)alkyl, halo(C₁-C₄)alkyl, and —C(O)NR^{a}R^{b};
   R¹⁵ is hydrogen, (C₁-C₄)alkyl, or halo(C₁-C₄)alkyl;
   W is 5- or 6-membered heteroaryl optionally substituted with 1 to 3 groups selected from R²;
   V is phenyl or 5- to 9-membered heteroaryl optionally substituted with 1 to 3 groups selected from R³;
   R¹ is halo, (C₁-C₄)alkyl, halo(C₁-C₄)alkyl, (C₁-C₄)alkoxy, or halo(C₁-C₄)alkoxy;
   R² is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{a}, —OR^{a}, —C(O)R^{a}, —C(O)OR^{a}, —C(O)NR^{a}R^{b}, —C(O)NR^{a}(C₁₋₄alkylene)OR^{a}, —C(O)NR^{a}(C₁₋₄alkylene)NR^{a}R^{b}, —C(O)NR^{a}(C₁₋₄alkylene)OR, —NR^{a}R^{b}, —O(C₁₋₄alkylene)NR^{a}R^{b}, —SH, —S(C₁₋₄alkyl), —C₁₋₄alkylNR^{a}R^{b}, —SR^{a}, —S(O)R^{a}, —S(O)₂R^{a}, —S(O)NR^{a}R^{b}, —SO₂NR^{a}R^{b}, —NR^{a}(C₁₋₄alkyl)OR^{a}, —NR^{a}(C₁₋₄alkyl)NR^{a}R^{b}, —C₁₋₆alkylC(O)NR^{a}R^{b}, phenyl or 5- to 7-membered heteroaryl, wherein said phenyl and 5- to 7-membered heteroaryl are each optionally and independently substituted with 1 to 3 groups selected from R⁴;
   R^{a} and R^{b} are each independently selected from hydrogen and (C₁-C₄)alkyl, wherein said (C₁-C₄)alkyl is optionally substituted with one or more halo or a 3- to 7-membered heterocyclyl, or both; and
   R³ and R⁴ are each independently halo, —NR^{a}R^{b}, (C₁-C₄)alkyl, halo(C₁-C₄)alkyl, (C₁-C₄)alkoxy, or halo(C₁-C₄)alkoxy, wherein the remaining variables are as described above for Formula I.

In a third embodiment, A in the compound of Formula I is

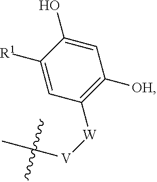

wherein the remaining variables are as described above for Formula I or the second embodiment. Alternatively, as part of a third embodiment, A in the compound of Formula I is selected from the structure corresponding to the position A in exemplified compounds 187 to 351, wherein the remaining variables are as described above for Formula I or the second embodiment.

In a fourth embodiment, the compound of Formula I is of the Formula

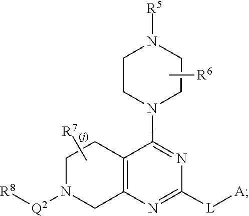

or a pharmaceutically acceptable salt thereof, wherein the remaining variables are as described above for Formula I or the second, or third embodiment.

In a fifth embodiment, R⁵ is —C(O)Y, wherein the remaining variables are as described above for Formula I or the second, third, or fourth embodiment.

In a sixth embodiment, Y is (C₁-C₆)alkyl, halo(C₁-C₆)alkyl, (C₂-C₆)alkenyl, halo(C₂-C₆)alkenyl, or NH₂, wherein the remaining variables are as described above for Formula I or the second, third, fourth, or fifth embodiment. Alternatively, as part of a sixth embodiment, Y is C(O)CH₃, C(O)CHCH₂, C(O)CH₂CH₃, C(O)CF₃, C(O)CFCH₂, C(O)CCH₃, or C(O)NH₂, wherein the remaining variables are as described above for Formula I or the second, third, fourth, or fifth embodiment. Alternatively, as part of a sixth embodiment, Y is C(O)CHCH₂, wherein the remaining variables are as described above for Formula I or the second, third, fourth, or fifth embodiment.

In a seventh embodiment, R⁶ is cyano(C₁-C₆)alkyl, wherein the remaining variables are as described above for Formula I or the second, third, fourth, fifth, or sixth embodiment. Alternatively, as part of a seventh embodiment, R⁶ is CH₂CN, wherein the remaining variables are as described above for Formula I or the second, third, fourth, fifth, or sixth embodiment.

In an eighth embodiment, j is 0, wherein the remaining variables are as described above for Formula I or the second, third, fourth, fifth, sixth, or seventh embodiment.

In an ninth embodiment, Q² is a bond, wherein the remaining variables are as described above for Formula I or the second, third, fourth, fifth, sixth, seventh, or eighth embodiment.

In a tenth embodiment, R⁸ is aryl optionally substituted with 1 to 3 groups selected from R⁹, wherein the remaining variables are as described above for Formula I or the second, third, fourth, fifth, sixth, seventh, eighth, or ninth embodiment. Alternatively, as part of a tenth embodiment, R⁸ is naphthyl optionally substituted with 1 to 3 groups selected from R⁹, wherein the remaining variables are as described above for Formula I or the second, third, fourth, fifth, sixth, seventh, eighth, or ninth embodiment.

In an eleventh embodiment, R⁹ is selected from halo, (C₁-C₆)alkyl, and OH, wherein the remaining variables are as described above for Formula I or the second, third, fourth, fifth, sixth, seventh, eighth, ninth, or tenth embodiment. Alternatively, as part of an eleventh embodiment, R⁹ is selected from chloro and OH, wherein the remaining variables are as described above for Formula I or the second, third, fourth, fifth, sixth, seventh, eighth, ninth, or tenth embodiment.

In a twelfth embodiment, A is selected from

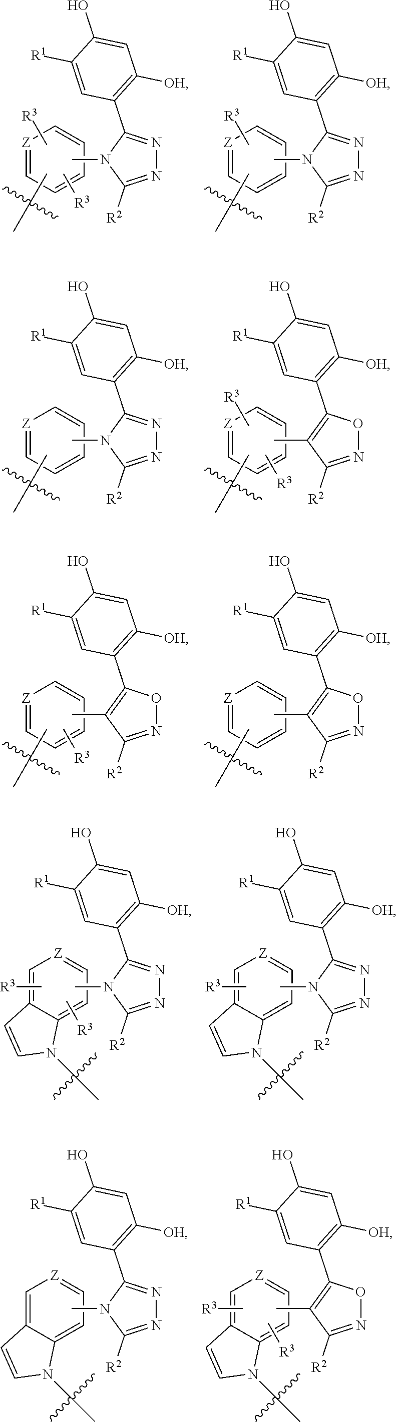

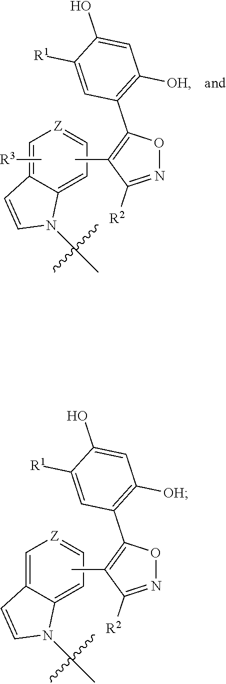

and Z is N or CH, wherein the remaining variables are as described above for Formula I or the second, third, fourth, fifth, sixth, seventh, eighth, ninth, tenth, or eleventh embodiment. Alternatively, as part of a twelfth embodiment, A is selected from the formula above; and Z is CH, wherein the remaining variables are as described above for Formula I or the second, third, fourth, fifth, sixth, seventh, eighth, ninth, tenth, or eleventh embodiment.

In a thirteenth embodiment, R³ is independently (C₁-C₄)alkyl or halo, wherein the remaining variables are as described above for Formula I or the second, third, fourth, fifth, sixth, seventh, eighth, ninth, tenth, eleventh, or twelfth embodiment.

In a fourteenth embodiment, A is

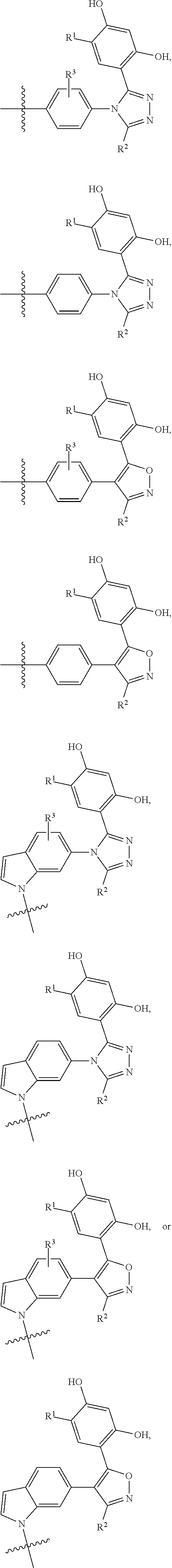

wherein the remaining variables are as described above for Formula I or the second, third, fourth, fifth, sixth, seventh, eighth, ninth, tenth, eleventh, twelfth, or thirteenth embodiment. Alternatively, as part of a fourteenth embodiment, A is

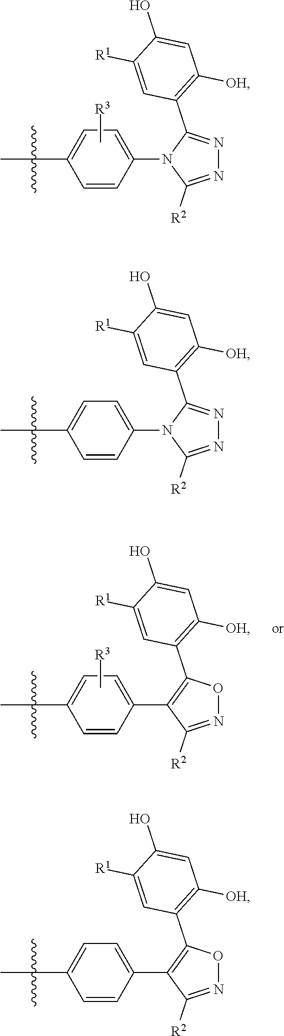

wherein the remaining variables are as described above for Formula I or the second, third, fourth, fifth, sixth, seventh, eighth, ninth, tenth, eleventh, twelfth, or thirteenth embodiment. Alternatively, as part of a fourteenth embodiment, A is

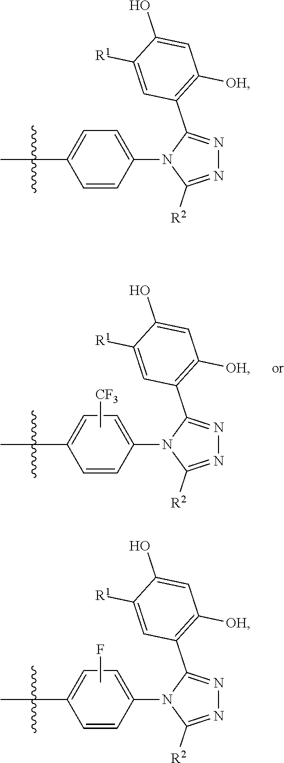

wherein the remaining variables are as described above for Formula I or the second, third, fourth, fifth, sixth, seventh, eighth, ninth, tenth, eleventh, twelfth, or thirteenth embodiment. Alternatively, as part of a fourteenth embodiment, A is

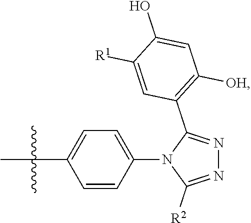

wherein the remaining variables are as described above for Formula I or the second, third, fourth, fifth, sixth, seventh, eighth, ninth, tenth, eleventh, twelfth, or thirteenth embodiment. In another alternative, as part of a fourteenth embodiment, is selected from the structures corresponding to position A in exemplified compounds 187 to 351, wherein the remaining variables are as described above for Formula I or the second, third, fourth, fifth, sixth, seventh, eighth, ninth, tenth, eleventh, twelfth, or thirteenth embodiment.

In a fifteenth embodiment, R¹ is halo or (C₁-C₄)alkyl, wherein the remaining variables are as described above for Formula I or the second, third, fourth, fifth, sixth, seventh, eighth, ninth, tenth, eleventh, twelfth, thirteenth, or fourteenth embodiment. Alternatively, as part of a fifteenth embodiment, R¹ is chloro, isopropyl, methyl, propyl, or ethyl, wherein the remaining variables are as described above for Formula I or the second, third, fourth, fifth, sixth, seventh, eighth, ninth, tenth, eleventh, twelfth, thirteenth, or fourteenth embodiment. Alternatively, as part of a fifteenth embodiment, R¹ is chloro, isopropyl or ethyl, wherein the remaining variables are as described above for Formula I or the second, third, fourth, fifth, sixth, seventh, eighth, ninth, tenth, eleventh, twelfth, thirteenth, or fourteenth embodiment.

In a sixteenth embodiment, R² is —OR^{a}, —SR^{a}, —C(O)NR^{a}R^{b}, or —C(O)NR^{a}(C₁₋₄alkylene)NR^{a}R^{b}, wherein the remaining variables are as described above for Formula I or the second, third, fourth, fifth, sixth, seventh, eighth, ninth, tenth, eleventh, twelfth, thirteenth, fourteenth, or fifteenth embodiment.

In a seventeenth embodiment, R^{a} and R^{b} are each independently selected from hydrogen and (C₁-C₄)alkyl, wherein said (C₁-C₄)alkyl is optionally substituted with 1 to 3 halo or a 6-membered heterocyclyl, wherein the remaining variables are as described above for Formula I or the second, third, fourth, fifth, sixth, seventh, eighth, ninth, tenth, eleventh, twelfth, thirteenth, fourteenth, fifteenth, or sixteenth embodiment.

In an eighteenth embodiment, R² is OH, —C(O)NHCH₂CF₃, —C(O)NHCH₂CH₃, —C(O)NHCH(CH₃)₂, —C(O)NH(CH₂CH₃)₂, —C(O)NHCH(CH₃)CF₃, —C(O)NHcyclopropyl, —C(O)NHmethylcyclopropyl, C(O)NH₂, or —C(O)NH(CH₂)₂piperidinyl, wherein the remaining variables are as described above for Formula I or the second, third, fourth, fifth, sixth, seventh, eighth, ninth, tenth, eleventh, twelfth, thirteenth, fourteenth, fifteenth, sixteenth, or seventeenth embodiment. Alternatively, as part of an eighteenth embodiment, R² is —C(O)NHCH₂CF₃ or OH, wherein the remaining variables are as described above for Formula I or the second, third, fourth, fifth, sixth, seventh, eighth, ninth, tenth, eleventh, twelfth, thirteenth, fourteenth, fifteenth, sixteenth, or seventeenth embodiment. Alternatively, as part of an eighteenth embodiment, R² is OH, wherein the remaining variables are as described above for Formula I or the second, third, fourth, fifth, sixth, seventh, eighth, ninth, tenth, eleventh, twelfth, thirteenth, fourteenth, fifteenth, sixteenth, or seventeenth embodiment.

In a nineteenth embodiment, L is selected from -Het¹-X¹—, -Het¹-, -Het¹-Het²-X¹—, -Het¹-Het²-, —NR^{d}—(CH₂)ₘ—X³—NR^{c}—(CH₂)ₘ-Het¹-X¹-Het²-X²—, —NR^{c}—(CH₂)ₘ-Het¹-X¹-Het²-X²—, -Het¹-X¹-Het²-X²—, O—(CH₂)ₘ—NR^{c}—X¹—(CH₂)ₘ—NR^{d}—, —X¹—NR^{c}—X²—O—(CH₂)ₘ—NR^{d}—, —X¹-Het¹-X²-Het²-(CH₂)ₘO—, O-Het¹-, O-Het¹-X¹—, —X¹(OCH₂CH₂)ₙ—NR^{c}—, —(CH₂)ₘNR^{c}—, —(CH₂)ₘ—, —O—, X¹NR^{c}—, —NR^{c}—(CH₂)ₘ—X¹-Het¹-X²—, —NR^{d}—(CH₂)ₘ—X³—NR^{c}—(CH₂)ₘ- Het¹-X¹-Het²-X²—, O-Het¹-X¹—(CH₂)ₘ—NR^{d}—, —X¹—NR^{c}—X²—(CH₂)ₘ—NR^{d}—, X¹-Het¹-X²—NR^{c}—X³-Het²-(OCH₂CH₂)ₙ—(CH₂)ₘ—NR^{d}—(CH₂)ₘ—, —NR^{d}—(CH₂)ₘ—X¹—NR^{c}—(CH₂CH₂O)ₙ—, —NR^{c}—(CH₂)ₘ—X¹—NR^{c}—(CH₂)ₚ—, X¹-Het¹-X²—NR^{c}—X³-Het²-(OCH₂CH₂)ₙ—NR^{d}—(CH₂)ₘ—, —NR^{c}—(CH₂)ₘ—X¹-Het¹-X²-Het²-X³—, O—X¹—Het¹-, —O(CH₂)ₘ—X¹-Het¹-X²-Het²-X³—, —O(CH₂)ₘ—X¹—NR^{c}—(CH₂)ₚ-Het¹-X²-Het²-X³—, O—(CH₂)ₘ—NR^{c}—, O—X¹-Het¹-X²—, —X¹—NR^{c}—(CH₂)ₘ-Het¹-X²-Het²-X³—(CH₂)ₚ—NR^{d}—(CH₂)ₚ—, —NR^{c}—(CH₂)ₘ—X¹—(CH)CH₃-Het¹-X²-Het³-X³—, —NR^{c}—(CH₂)ₘ—X¹—(CH₂)ₚ-Het¹-X²-Het²-X³—, —NR^{c}—(CH₂)ₘ—X¹—NR^{d}—(CH₂)ₚ-Het¹-X²-Het²-X³—, —NR^{c}—(CH₂)ₘ—NR^{d}—X¹-Het¹-X²—, Het¹-X¹-Het²-X²—, -Het¹-X¹-Het²-X²—O—, —O(CH₂)ₘ-Het¹-(CH₂)ₚ—O(CH₂)ₘ—NR^{c}—X²—, —O(CH₂)ₘ-Het¹-(CH₂)ₚ—O(CH₂)ₘ—NR^{c}—X²—, -Het¹-O—(CH₂)ₘ X¹-Het²-X²—, -Het¹-O—(CH₂)ₘ—X¹—NR^{c}—(CH₂CH₂O)ₙ(CH₂)ₘ-Het²-X²—, -Het¹-X¹—NR^{c}—(CH₂)ₘ—, -Het¹-X¹-Het²-Het³-X²—, -Het¹-X¹—NR^{c}—(CH₂CH₂O)ₙ(CH₂)ₘ—, -Het¹-X¹—NR^{c}—(CH₂CH₂O)ₙHet²-(CH₂)ₘ—X²—, -Het¹-X¹—NR^{c}—(CH₂CH₂O)ₙ—, -Het¹-X¹—NR^{c}—(CH₂)ₘ-Het²-X²-Het³-(CH₂)ₘ—, -Het¹-X¹-Het²-(CH₂)ₘ Het³-X²—, -Het¹-X¹-Het²-, -Het¹-X¹—NR^{c}—, -Het¹-X¹—NR^{c}—(CH₂)ₘ-Phe-X²-Het²-(CH₂)ₘ, -Het¹-X¹-Het²-Het³-, -Het¹-X¹-Het²-(CH₂)ₘ-Het³-X²—(CH₂)ₚ—NR^{c}—(CH₂)ₘ—, -Het¹-X¹-Het²-(CH₂)ₘ-Het³-(CH₂)ₘO—, -Het¹-X¹-Het²-(CH₂)ₘ-Het³-(CH₂)ₚ—NR^{c}—(CH₂)ₘ—, -Het¹-X¹-Het²-(CH₂CH₂O)ₙ—, -Het¹-X¹—(CH₂)ₘ-Het²-X²—, —(CH₂CH₂O)ₒ—(CH₂)ₚ-Het¹-X¹-Het²-(CH₂CH₂O)ₙ, —(CH₂CH₂O)ₙ—(CH₂)ₘ-Het¹-X¹-Het²-X², -Het¹-X¹-Phe-X²—NR^{c}—X³—, —(CH₂CH₂O)ₒ—(CH₂)ₚ-Het¹-X¹-Phe-X²—NR^{c}—(CH₂CH₂O)ₙ—, —(CH₂CH₂O)ₙ—(CH₂)ₘ—NR^{c}-Phe-X¹—, —(CH₂CH₂O)ₒ—(CH₂)ₚ—NR^{c}-Phe-(CH₂CH₂O)ₙ—, —(CH₂CH₂O)ₒ—(CH₂)ₚ—NR^{c}—(CH₂CH₂O)ₙ—(CH₂)ₘ—, —(CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, —(CH₂CH₂O)ₒ—(CH₂)ₚ—NR^{c}—(CH₂CH₂O)ₙ—(CH₂)ₘ-Het¹-X¹-Het²-X²—, —(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, —NR^{c}—(CH₂CH₂O)ₙ—(CH₂)ₘ- Phe-NH—X¹-Het¹-X², —NR^{c}—(CH₂CH₂O)ₙ—(CH₂)ₘ-Phe-NH—X¹-Het¹-X²—(CH₂CH₂O)ₒ, —(CH₂CH₂O)ₒ, —(C₂CH₂)ₚ—NR^{c}—(CH₂CH₂O)ₙ—(CH₂)ₘ-Phe-X¹—NR^{c}—(CH₂CH₂O)ₒ—(CH₂)ₚ—, —(CH₂CH₂O)ₒ—(CH₂)ₚ—NR^{c}—(CH₂CH₂O)ₙ—(CH₂)ₘ-Het¹-X¹—, —(CH₂CH₂O)ₒ—(CH₂)ₚ—NR^{c}—(CH₂CH₂O)ₙ—(CH₂)ₘ-Het¹-X¹—(CH₂CH₂O)ₙ—, —(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ, or —NR^{c}—(CH₂)ₘ—C(O)—NR^{d}—(CH₂)ₘ-Het¹-X¹-Het²-X²—, C(O)O-—X¹-Het¹-(CH₂CH₂O)ₒ—(CH₂)ₘ—NR^{c}—, -Het¹-(CH₂)ₘ-Het²-, -Het¹-X¹-Het²-(CH₂)ₚ—O—(CH₂)ₘ—, O(CH₂)ₘC(O), —OC(O)—NR^{c}—(CH₂)ₘ—NR^{d}—, —OC(O)—NR^{c}—(CH₂)ₘ—O(CH₂)ₘ—NR^{d}—, OC(O)Het¹, —OC(O)—NR^{c}—(CH₂CH₂O)ₒ—NR^{d}—, OC(O)Het¹-Het²-, —OC(O)—NR^{c}—(CH₂)ₘC(O)-Het¹-X¹-Het²-, O—(CH₂)ₘ-Het¹-, and O—(CH₂)ₘ-Het¹-X¹-Het²;
Het¹, Het², and Het³ are each independently phenyl, a 4- to 6-membered heterocyclyl, 5- to 7-membered heteroaryl, or a 4- to 6-membered cycloalkyl, each of which are optionally substituted with (C₁-C₄)alkyl;
X¹, X², and X³, are each independently C(O) or (CH₂)ᵣ; and
m, n, o, p, q and r are each independently integers selected from 0, 1, 2, 3, 4, 5, and 6, wherein the remaining variables are as described above for Formula I or the second, third, fourth, fifth, sixth, seventh, eighth, ninth, tenth, eleventh, twelfth, thirteenth, fourteenth, fifteenth, sixteenth, seventeenth, or eighteenth embodiment. Alternatively, as part of a nineteenth embodiment, L is selected from -Het¹-X¹—, -Het¹-Het²-X¹—, —X¹-Het¹-X²-Het²-(CH₂)ₘO—, —NR^{c}—(CH₂)ₘ—X¹-Het¹-X²—, —NR^{c}—(CH₂)ₘ—X¹—NR^{c}—(CH₂CH₂O)ₙ—, —NR^{c}—(CH₂)ₘ X¹—NR^{c}—(CH₂)ₚ—, —NR^{c}—(CH₂)ₘ—X¹-Het¹-X²-Het²-X³—, —O(CH₂)ₘ—X¹-Het¹-X²-Het²- X³—, —O(CH₂)ₘ—X¹—NR^{c}—(CH₂)ₚ-Het¹-X²-Het²-X³—, —X¹—NR^{c}—(CH₂)ₘ-Het¹-X²-Het²-X³—(CH₂)ₚ—NR^{d}—(CH₂)ₚ—, —NR^{c}—(CH₂)ₘ—X¹—(CH)CH₃-Het¹-X²-Het³-X³—, —NR^{c}—(CH₂)ₘ—X¹—(CH₂)ₚ- Het¹-X²-Het²-X³—, —NR^{c}—(CH₂)ₘ—X¹—NR^{d}—(CH₂)ₚ-Het¹-X²-Het²-X³—, —NR^{c}—(CH₂)ₘ NR^{c}—X¹-Het¹-X²-Het¹-X¹-Het²-X²—, -Het¹-X¹-Het²-X²—O—, —O(CH₂)ₘ-Het¹-(CH₂)ₚ—O(CH₂)ₘ—NR^{c}—X²—, -Het¹-O—(CH₂)ₘ—X¹-Het²-X²—, -Het¹-O—(CH₂)ₘ—X¹—NR^{c}—(CH₂CH₂O)ₙ(CH₂)ₘ-Het²-X²—, -Het¹- X¹—NR^{c}—(CH₂)ₘ—, -Het¹-X¹-Het²-Het³-X²—, -Het¹-X¹—NR^{c}—(CH₂CH₂O)ₙ(CH₂)ₘ—, -Het¹-X¹—NR^{c}—(CH₂CH₂O)ₙHet²-(CH₂)ₘ—X²—, -Het¹-X¹—NR^{c}—(CH₂CH₂O),₁-, -Het¹-X¹—NR^{c}—(CH₂)ₘ-Het²-X²-Het³-(CH₂)ₘ—, -Het¹-X¹-Het²-(CH₂)ₘ-Het³-X²—, -Het¹-X¹-Het²-, -Het¹-X¹—NR^{c}—, -Het¹-X¹—NR^{c}—(CH₂)ₘ-Phe-X²-Het²-(CH₂)ₘ—, -Het¹-X¹-Het²-Het³-, -Het¹-X¹-Het²-(CH₂)ₘ-Het³-X²—(CH₂)ₚ—NR^{c}—(CH₂)ₘ—, -Het¹-X¹-Het²-(CH₂)ₘ-Het³-(CH₂)ₘ—O—, -Het¹-X¹-Het²-(CH₂)ₘ-Het³-(CH₂)ₚ—NR^{c}—(CH₂)ₘ—, -Het¹-X¹-Het²-(CH₂CH₂O)ₙ—, -Het¹-X¹—(CH₂)ₘ-Het²-X²—, —(CH₂CH₂O)ₒ—(CH₂)ₚ-Het¹-X¹-Het²-(CH₂CH₂O)ₙ, —(CH₂CH₂O)ₙ—(CH₂)ₘ-Het¹-X¹-Het²-X², -Het¹-X¹-Phe-X²—NR^{c}—X³—, —(CH₂CH₂O)ₒ—(CH₂)ₚ-Het¹-X¹-Phe-X²—NR^{c}—(CH₂CH₂O)ₙ—, —(CH₂CH₂O)ₙ—(CH₂)ₘ—NR^{c}-Phe-X¹—, —(CH₂CH₂O)ₒ—(CH₂)ₚ—NR^{c}-Phe-(CH₂CH₂O)ₙ—, —(CH₂CH₂O)ₒ—(CH₂)ₚ—NR^{c}—(CH₂CH₂O)ₙ—(CH₂)ₘ—, —(CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, —(CH₂CH₂O)ₒ—(CH₂)ₚ—NR^{c}—(CH₂CH₂O)ₙ—(CH₂)ₘ Het¹-X¹-Het²-X²—, —(CH₂CH₂O)ₒ—(CH₂)ₚ—NR^{c}—(CH₂CH₂O)ₙ—(CH₂)ₘ-Het¹-X¹-Het²-X²—(CH₂CH₂O)ₒ, —NR^{c}—(CH₂CH₂O)ₙ—(CH₂)ₘ-Phe-NH—X¹-Het¹-X², —NR^{c}—(CH₂CH₂O)ₙ—(CH₂)ₘ Phe-NH—X¹-Het¹-X²—(CH₂CH₂O)ₒ, —(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ, —(CH₂)ₚ—, —(CH₂CH₂O)ₒ—(CH₂)ₚ—NR^{c}—(CH₂CH₂O)ₙ—(CH₂)ₘ-Het¹-X¹—, —(CH₂CH₂O)ₒ—(CH₂)ₚ—NR^{c}—(CH₂CH₂)ₙ—(CH₂)ₘ-Het¹-X¹—(CH₂CH₂O)ₙ—, —(CH₂CH₂O)ₙ—(CH₂)ₘ—NR^{c}—(CH₂)ₘ—C(O)—NR^{d}-Het¹-X¹-Het²-(CH₂CH₂O)ₒ—(CH₂)ₚ, and —NR^{c}—(CH₂)ₘ—C(O)—NR^{d}—(CH₂)ₘ-Het¹-X¹-Het²-X²—, wherein the remaining variables are as described above for Formula I or the second, third, fourth, fifth, sixth, seventh, eighth, ninth, tenth, eleventh, twelfth, thirteenth, fourteenth, fifteenth, sixteenth, seventeenth, or eighteenth embodiment. Alternatively, as part of a nineteenth embodiment, L is -Het¹-X¹—*, -Het¹-Het²-X¹—*, *—X¹-Het¹-X²-Het²-(CH₂)ₘO—, —NR^{c}—(CH₂)ₘ—X¹-Het¹-X²—*, —NR^{c}—(CH₂)ₘ—X¹—NR^{c}—(CH₂CH₂O)ₙ—*, —NR^{c}—(CH₂)ₘ—X¹—NR^{c}—(CH₂)ₚ—*, —NR^{c}—(CH₂)ₘ—X¹-Het¹-X²-Het²-X³—*, —O(CH₂)ₘ—X¹-Het¹-X²-Het²-X³—*, —O(CH₂)ₘ—X¹—NR^{c}—(CH₂)ₚ-Het¹-X²-Het²-X³—*, *—X¹—NR^{c}—(CH₂)ₘ-Het¹-X²-Het²-X³—(CH₂)ₚ—NR^{d}—(CH₂)ₚ—, —NR^{c}—(CH₂)ₘ—X¹—(CH)CH₃—Het¹-X²-Het³-X³—*, —NR^{c}—(CH₂)ₘ—X¹—(CH₂)ₚ-Het¹-X²-Het²-X³—*, —NR^{c}—(CH₂)ₘ—X¹—NR^{d}—(CH₂)ₚ-Het¹-X²- Het²-X³—*, —NR^{c}—(CH₂)ₘ NR^{d}—X¹-Het¹-X²—*, *Het¹-X¹-Het²-X²—, *-Het¹-X¹-Het²-X²—O—, *—O(CH₂)ₘ-Het¹-(CH₂)ₚ—O(CH₂)ₘ—NR^{c}—X²—, —O(CH₂)ₘ-Het¹-(CH₂)ₚ—O(CH₂)ₘ—NR^{c}—X²—*-Het¹-O—(CH₂)ₘ—X¹-Het²-X²—, *-Het¹-O—(CH₂)ₘ—X¹—NR^{c}—(CH₂CH₂O)ₙ(CH₂)ₘ-Het²-X²—, *-Het¹-X¹—NR^{c}—(CH₂)ₘ—, *-Het¹-X¹-Het²-Het³-X²—, *-Het¹-X¹—NR^{c}—(CH₂CH₂O)ₙ(CH₂)ₘ—, *-Het¹-X¹—NR^{c}—(CH₂CH₂O)ₙHet²-(CH₂)ₘ X²—, *-Het¹-X¹—NR^{c}—(CH₂CH₂O)ₙ—, *-Het¹-X¹—NR^{c}—(CH₂)ₘ Het²-X²-Het³-(CH₂)ₘ—, *-Het¹-X¹-Het²-(CH₂)ₘ-Het³-X²—, *-Het¹-X¹-Het²-, *-Het¹-X¹—NR^{c}—, *-Het¹-X¹—NR^{c}—(CH₂)ₘ-Phe-X²-Het²-(CH₂)ₘ—, *-Het¹-X¹-Het²-Het³-, *-Het¹-X¹-Het²-(CH₂)ₘ-Het³-X²—(CH₂)ₚ—NR^{c}—(CH₂)ₘ—, *-Het¹-X¹-Het²-(CH₂)ₘ-Het³-(CH₂)ₘ—O—, *-Het¹-X¹-Het²-(CH₂)ₘ-Het³-(CH₂)ₚ—NR^{c}—(CH₂)ₘ—, *-Het¹-X¹-Het²-(CH₂CH₂O)ₙ—,*-Het¹-X¹—(CH₂)ₘ-Het²-X²—, *—(CH₂CH₂O)ₒ—(CH₂)ₚ-Het¹-X-Het²-(CH₂CH₂O)ₙ, *—(CH₂CH₂O)ₙ—(CH₂)ₘ-Het¹-X¹-Het²-X², *-Het¹-X¹-Phe-X²—NR^{c}—X³—, *—(CH₂CH₂O)ₒ—(CH₂)ₚ-Het¹-X¹-Phe-X²—NR^{c}—(CH₂CH₂O)ₙ—, *—(CH₂CH₂O), —(CH₂)ₘ—NR^{c}-Phe-X¹—, *—(CH₂CH₂O)ₒ—(CH₂)ₚ—NR^{c}-Phe-(CH₂CH₂O)ₙ—, *—(CH₂CH₂O)ₒ—(CH₂)ₚ—NR^{c}—(CH₂CH₂O)ₙ—(CH₂)ₘ—, *—(CH₂CH₂O)ₙ—(CH₂)ₘ NR^{c}—(CH₂CH₂O)ₙ—(CH₂)ₘ—C(O)—NR^{d}—(CH₂CH₂O)ₒ—(CH₂)ₚ—, *—(CH₂CH₂O)ₒ—(CH₂)ₚ—NR^{c}—(CH₂CH₂O)ₙ—(CH₂)ₘ-Het¹-X¹-Het²-X²—, *—(CH₂CH₂O)ₒ—(CH₂)ₚ—NR^{c}—(CH₂CH₂O)ₙ—(CH₂)ₘ Het¹-X¹-Het²-X²—(CH₂CH₂O)ₒ, *—NR^{c}—(CH₂CH₂O)ₙ—(CH₂)ₘ-Phe-NH—X¹-Het¹-X², *—NR^{c}—(CH₂CH₂O)ₙ—(CH₂)ₘ-Phe-NH—X¹-Het¹-X²—(CH₂CH₂O)ₒ, *—(CH₂CH₂O)ₒ—(CH₂)ₚ—NR^{c}—(CH₂CH₂O)ₙ—(CH₂)ₘ-Phe-X¹—NR^{c}—(CH₂CH₂O)ₒ—(CH₂)ₚ—, *—(CH₂CH₂O)ₒ—(CH₂)ₚ—NR^{c}—(CH₂CH₂O)ₙ—(CH₂)ₘ-Het-X¹—, *—(CH₂CH₂O)ₒ—(CH₂)ₚ—NR^{c}—(CH₂CH₂O)ₙ—(CH₂)ₘ-Het¹-X¹—(CH₂CH₂O)ₙ—, *—(CH₂CH₂O)ₙ—(CH₂)ₘ—NR^{c}—(CH₂)ₘ—C(O)—NR^{d}-Het¹-X¹-Het²-(CH₂CH₂O)ₒ₋(CH₂)ₚ, or *—NR^{c}—(CH₂)ₘ—C(O)—NR^{d}—(CH₂)ₘ-Het¹-X¹-Het²-X²—; wherein * indicates the point of attachment to A and wherein the remaining variables are as described above for Formula I or the second, third, fourth, fifth, sixth, seventh, eighth, ninth, tenth, eleventh, twelfth, thirteenth, fourteenth, fifteenth, sixteenth, seventeenth, or eighteenth embodiment. Alternatively, as part of a nineteenth embodiment, L is -Het¹-X¹—*, -Het¹-Het²-X¹—*, *—X¹-Het¹-X²-Het²-(CH₂)ₘO—, —NR^{c}—(CH₂)ₘ—X¹-Het¹-X²—*, —NR^{c}—(CH₂)ₘ—X¹—NR^{c}—(CH₂CH₂O)ₙ—*, —NR^{c}—(CH₂)ₘ-X¹—NR^{c}—(CH₂)ₚ—*, —NR^{c}—(CH₂)ₘX¹-Het¹-X²-Het²-X³—*, —O(CH₂)ₘ—X¹-Het¹-X²-Het²-X³—*, —O(CH₂)ₘ—X¹—NR^{c}—(CH₂)ₚ-Het¹-X²-Het²-X³—*, *—X¹—NR^{c}—(CH₂)ₘ-Het¹-X²-Het²-X³—(CH₂)ₚ—NR^{d}—, —NR^{c}—(CH₂)ₘ X¹—(CH)CH₃-Het¹-X²-Het²-X³—*, —NR^{c}—(CH₂)ₘ—X¹—(CH₂)ₚ-Het¹-X¹-Het¹-X¹—*, —NR^{c}—(CH₂)ₘ—X¹—NR^{c}—(CH₂)ₚ-Het¹-X¹-Het¹-X³—*, —NR^{c}—(CH₂)ₘ—NR^{d}—X¹-Het¹-X²—*, *Het¹-X¹-Het²-X²—, *-Het¹-X¹-Het²-X²—O—, *—O(CH₂)ₘ-Het¹-(CH₂)ₚ—O(CH₂)ₘ—NR^{c}—X²—, or *-Het¹-X¹-Het²-, wherein the remaining variables are as described above for Formula I or the second, third, fourth, fifth, sixth, seventh, eighth, ninth, tenth, eleventh, twelfth, thirteenth, fourteenth, fifteenth, sixteenth, seventeenth, or eighteenth embodiment. Alternatively, as part of a nineteenth embodiment, L is —NR^{c}—(CH₂)ₘ—X¹—NR^{c}—(CH₂)ₚ—* or —NR^{c}—(CH₂)ₘ—X¹-Het¹-X²-Het²-X³—*, wherein the remaining variables are as described above for Formula I or the second, third, fourth, fifth, sixth, seventh, eighth, ninth, tenth, eleventh, twelfth, thirteenth, fourteenth, fifteenth, sixteenth, seventeenth, or eighteenth embodiment.

In a twentieth embodiment, Het¹ and Het² are each independently phenyl or a 4- to 6-membered heterocyclyl, wherein the remaining variables are as described above for the nineteenth embodiment. Alternatively, as part of a twentieth embodiment Het¹ and Het² are each independently piperidinyl, phenyl, azetidinyl, piperazinyl, or pyrrolidinyl, wherein the remaining variables are as described above for the nineteenth embodiment.

In a twenty-first embodiment, m, n, o, p, q and r are each independently integers selected from 0, 1, 2, and 3, wherein the remaining variables are as described above for the nineteenth or twentieth embodiment.

In a twenty-second embodiment, L is selected from

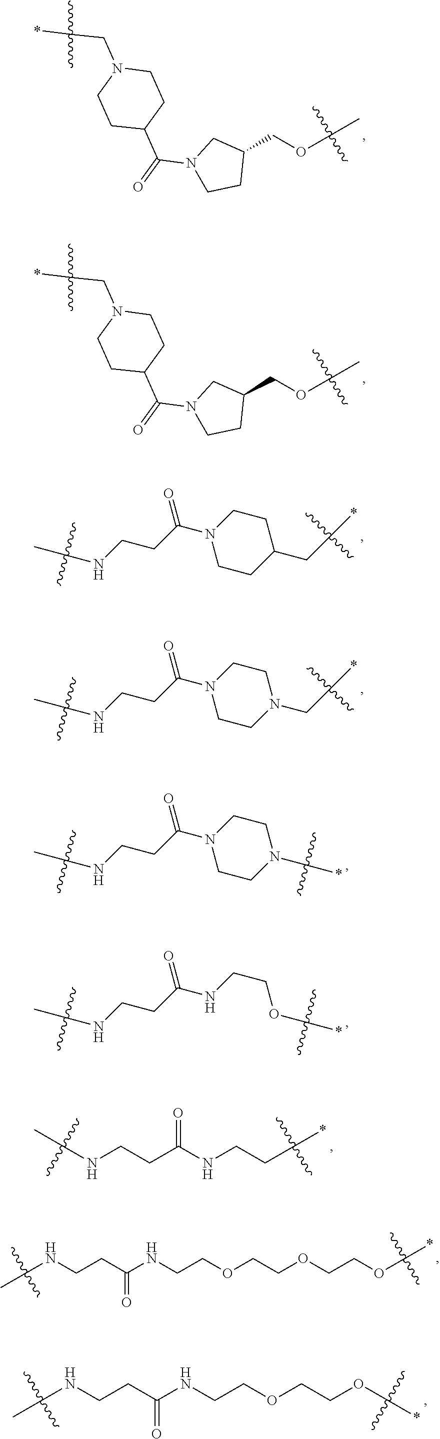

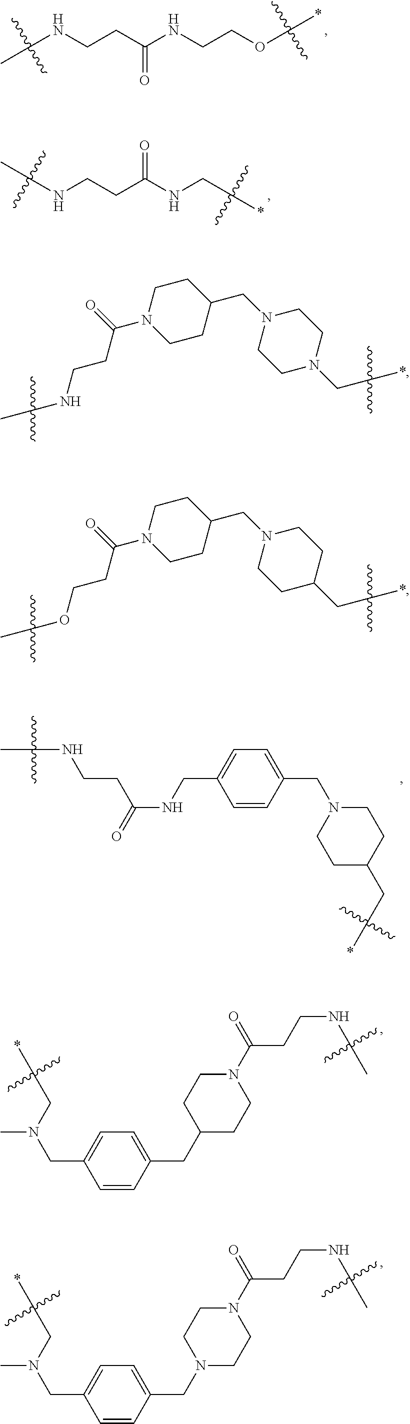

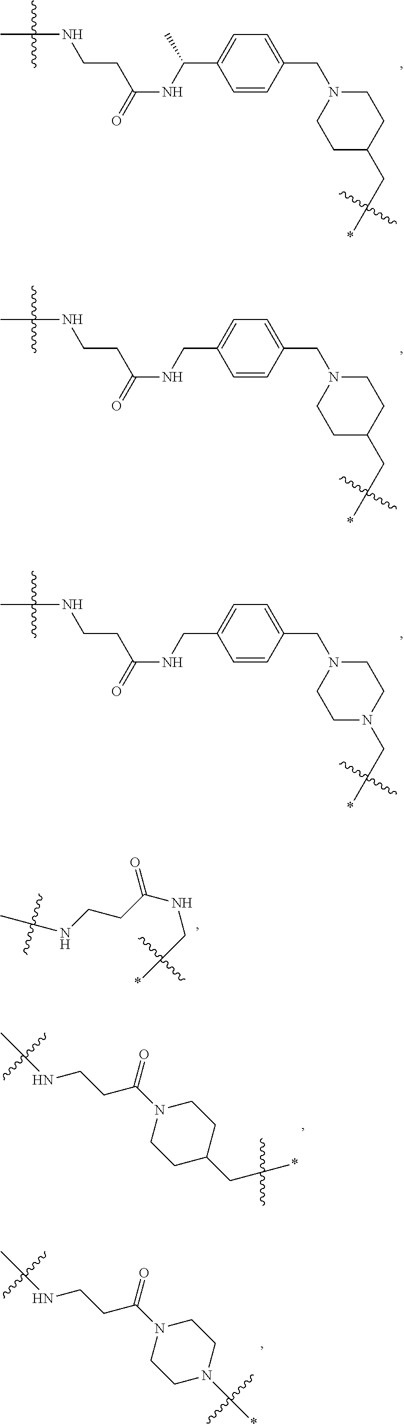

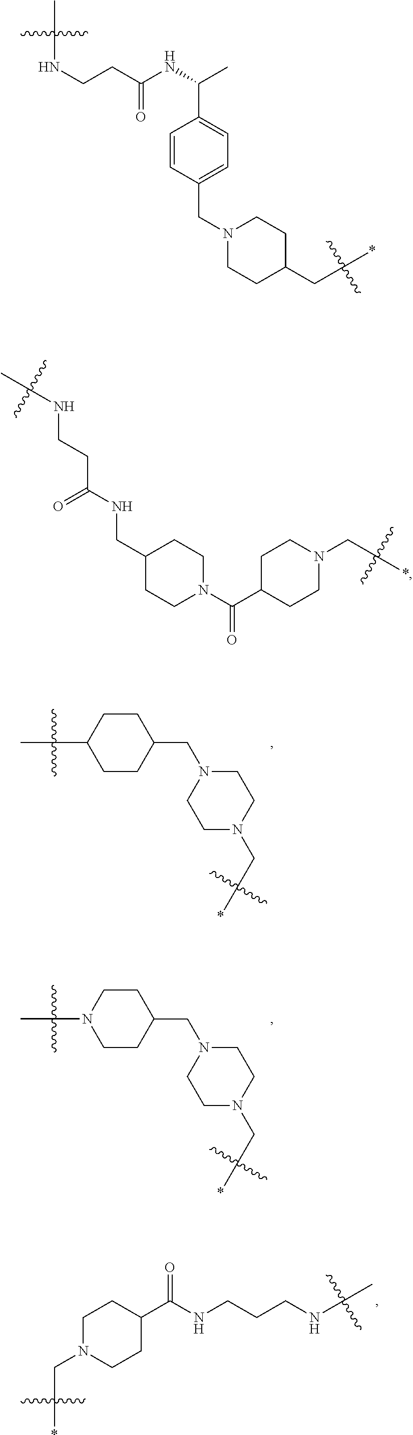

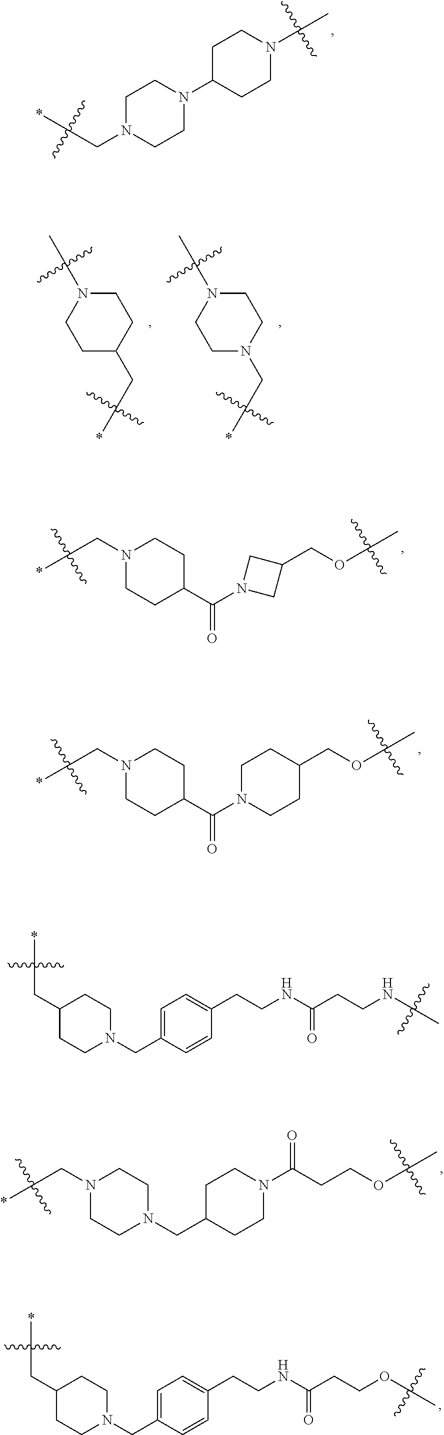

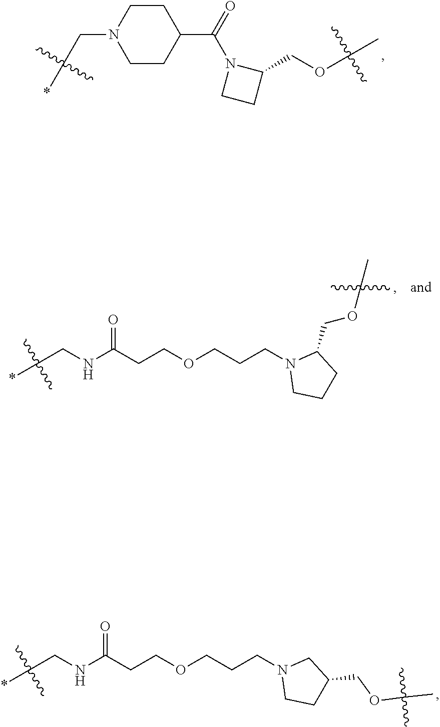

wherein the remaining variables are as described above for Formula I or the second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first embodiment. Alternatively, as part of a twenty-second embodiment, L is selected from

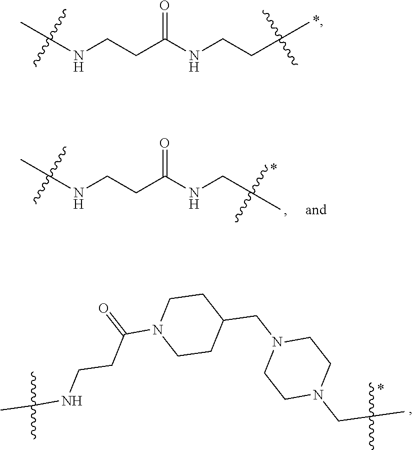 [SMILES: *CCNC(=O)CCN* ; *CNC(=O)CCN* ; *CN1CCN(CC2CCN(C(=O)CCN*)CC2)CC1]

wherein the remaining variables are as described above for Formula I or the second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first embodiment. In another alternative, as part of a twelfth embodiment, is selected from the structures corresponding to position L in exemplified compounds 187 to 351, wherein the remaining variables are as described above for Formula I or the second, third, fourth, fifth, sixth, seventh, eighth, ninth, tenth, or eleventh embodiment.

Specific compounds are exemplified below and are included as part of the invention. Free base and salt forms of these compounds are also included.

### 4. Uses, Formulation and Administration

Compounds and compositions described herein are generally useful as anticancer therapies. In one aspect, the disclosed compounds and compositions behave as chaperone-mediated protein degraders (CHAMPs) in which one portion of the compounds is responsible for binding KRAS(G12C) and the other portion is responsible for binding to HSP90 or other chaperone proteins or protein components of chaperone complexes (e.g., members of the HSP70 family). Their mechanisms of action include, but are not limited to, degrading KRAS(G12C) and thereby impeding down-stream signals that may result in inhibition of cancer cell growth and/or induction of cancer cell death or other KRAS or KRAS(G12C) functions. In one aspect, the disclosed compounds effectuate the degradation of KRAS(G12C).

In one aspect, the disclosed compounds and compositions include chaperone or chaperone complex binders that have a range of different binding affinities. In different embodiments, it is desirable to use a high-affinity binder, a moderate-affinity binder or a low-affinity binder. Since a HSP90-binding moiety that interacts with the N-terminal ATP-binding pocket of HSP90 may inhibit HSP90 activity and induce the degradation of HSP90 client proteins (Schopf et al., Nat Rev Mol Cell Biol, 2017, 18:345-360), some CHAMP molecules may not only induce the degradation of the desired target protein or proteins (which may or may not be HSP90 client proteins), but also simultaneously induce the degradation of HSP90 client proteins. EGFR and ERBB2 (HER2) are two such HSP90 client proteins (Xu et al., J Biol Chem, 2001, 276:3702-3708). Such combinations of degradation activities may increase the biological activity of CHAMP molecules over that of other TPD technologies directed towards the same target(s) and may evade mechanisms of resistance to KRAS(G12C) inhibitors and degraders such as that mediated by the EGFR pathway.

In one aspect, the disclosed compounds and compositions behave as tumor-targeted CHAMPs in which one portion of the compounds is responsible for binding KRAS(G12C) and the other portion is responsible for binding to HSP90 or other chaperone proteins or protein components of chaperone complexes (e.g., members of the HSP70 family). In one aspect, the disclosed compounds and compositions have prolonged pharmacokinetic exposures in cancer cells and tumors relative to normal cells, tissues and organs (Kamal et al., Nature, 2003, 425:407-410; Vilenchik et al., Chem Biol, 2004, 11:787-797). In one aspect, the disclosed compounds have increased therapeutic indexes relative to other KRAS(G12C) degraders and inhibitors.

Thus, provided herein are methods of treating conditions which are responsive to the degradation of KRAS(G12C) comprising administering to a subject in need thereof, a therapeutically effective amount of one or more compounds or compositions described herein. Also provided is the use of one or more compounds or compositions described herein in the manufacture of a medicament for treating conditions which are responsive to the degradation of KRAS(G12C). Further provided is the use of a compound or composition described herein for treating conditions which are responsive to the degradation of KRAS(G12C).

In one aspect, the condition treated by the present compounds and compositions is a cancer. The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin and non-Hodgkin), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” or “tumorous tissue” are understood as cells, extracellular matrix, and other naturally occurring components associated with the solid tumor.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### EXEMPLIFICATION

### Example 1: Compound 148 Synthesis

A representative synthesis scheme for compound 148 is shown in below. Specific synthesis routes of intermediates are also shown.

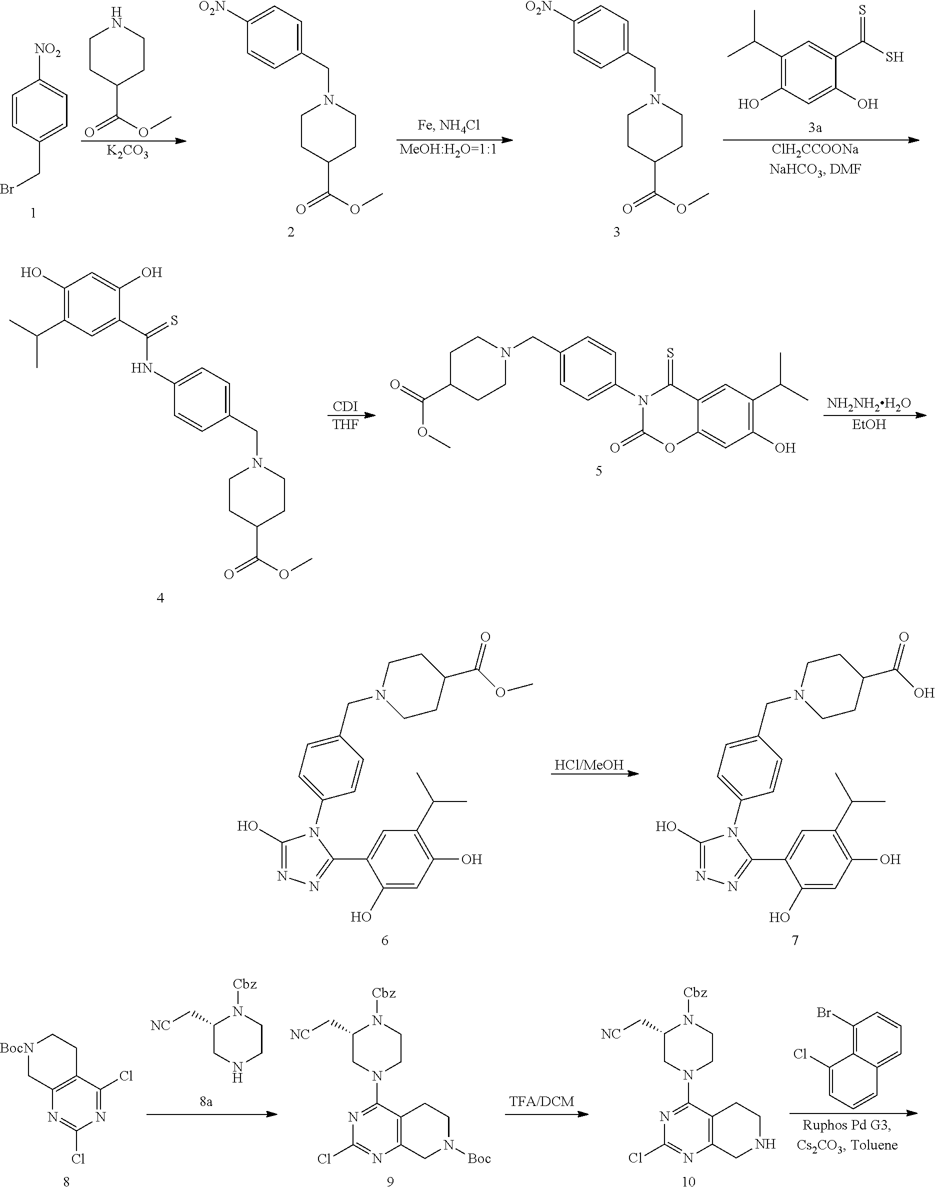

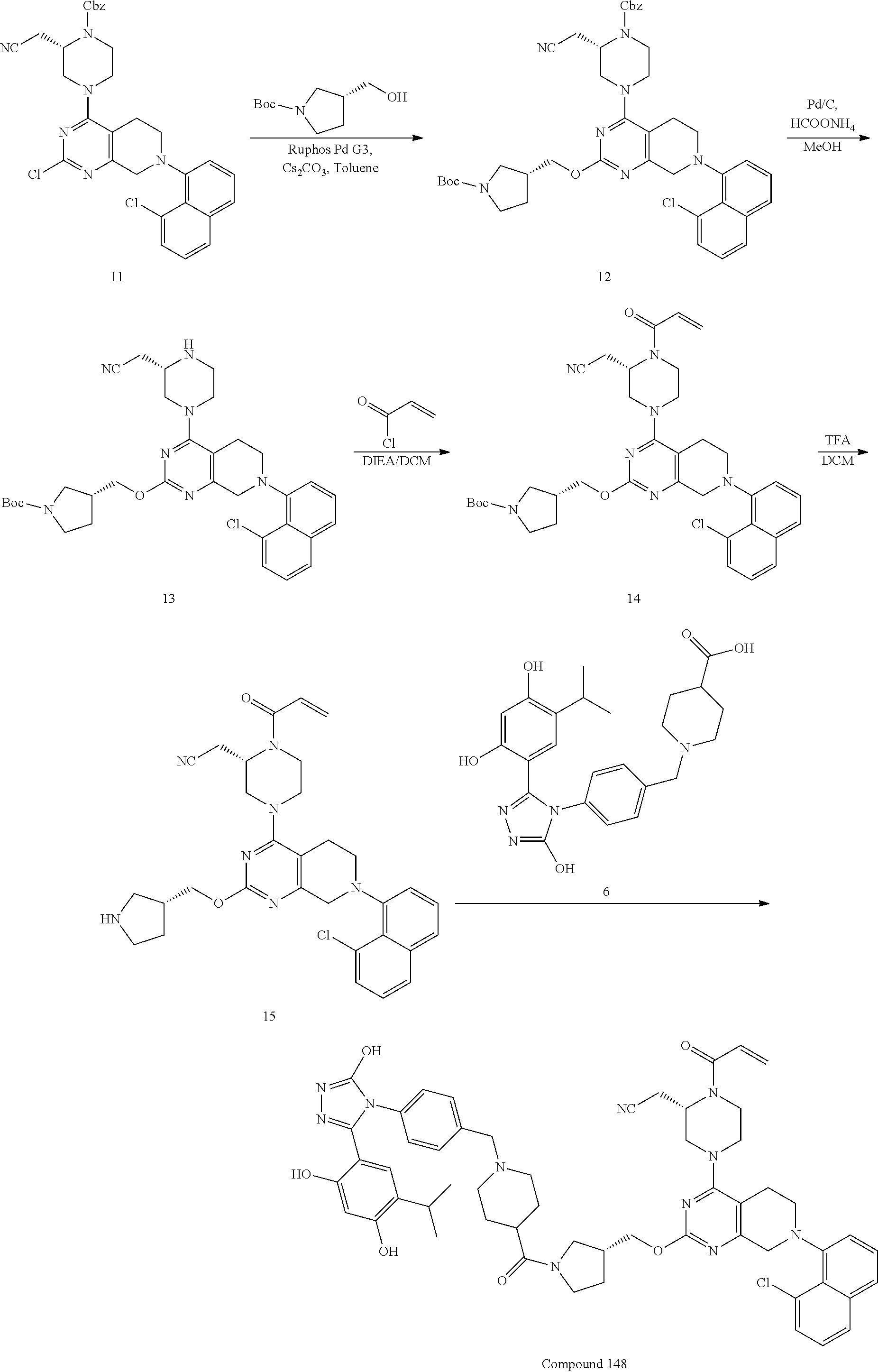

### Intermediate 2

### methyl 1-(4-nitrobenzyl)piperidine-4-carboxylate

To the mixture of 1-(bromomethyl)-4-nitrobenzene (40 g, 185.16 mmol) and methyl piperidine-4-carboxylate (26.51 g, 185.16 mmol) in DMF (300 mL) was added K2CO3 (51.18 g, 370.22 mmol). The mixture was stirred at r.t. overnight. The mixture was poured into 500 mL of H2O. It was extracted with EA (500 mL*2). Combined organic phase was washed with H2O and brine. It was dried (Na2SO4), filtered and concentrated to give t Intermediate 2 as yellow solid (49 g).

### Intermediate 3

### methyl 1-(4-aminobenzyl)piperidine-4-carboxylate

To the mixture of methyl 1-(4-nitrobenzyl)piperidine-4-carboxylate (5.0 g, 17.97 mmol) and NH₄Cl (4.81 g, 89.83 mmol) in MeOH/H₂O (25 mL/25 mL) was added Fe powder (5.02 g 89.83 mmol). The mixture was stirred at reflux overnight. After cooled to r.t., the mixture was filtered and the filtrate was concentrated. It was extracted with EA (50 mL*2). Combined organic phase was washed with H2O and brine. It was dried (Na2SO4), filtered and concentrated to give Intermediate 3 as brown oil (3.8 g)

### Intermediate 4

### Methyl 1-(4-(2,4-dihydroxy-5-isopropylphenylthioamido)benzyl)piperidine-4-carboxylate

To a solution of compound 3a (20.69 g, 90.61 mmol), ClCH₂COONa (10.55 g, 90.61 mmol) and NaHCO₃ (15.22 g, 181.22 mmol) in DMF (100 mL) was stirred at 40° C. for 3 hours. Intermediate 3 (15.0 g, 60.41 mmol) in DMF (50 mL) was added to the mixture. After the resulting mixture was heated at 80° C. overnight. The reaction mixture was poured into water and solid precipitated was collected by filtration. The mother liquid was extracted with EA (200 mL*2). Combined organic phase was washed with H2O and brine. It was dried (Na₂SO₄), filtered and concentrated. It was combined with the solid filtered and purified by SGC to give Intermediate 4 (22.1 g) as a yellow solid.

### Intermediate 5

### methyl 1-(4-(7-hydroxy-6-isopropyl-2-oxo-4-thioxo-2H-benzo[e][1,3]oxazin-3(4H)-yl)benzyl)piperidine-4-carboxylate

The solution of Intermediate 4 (8.0 g, 18.08 mmol) and CDI (11.72 g, 72.3 mmol) in THF (80 mL) was stirred for 2 hours at room temperature. The reaction solution was poured into brine (1 L) and extracted with EtOAc (200 mL*3). The combine organic layers was washed with brine, dried over Na₂SO₄ and concentrated to give the Intermediate 5 (crude) which was used for further reaction without purification.

### Intermediate 6

### Methyl 1-(4-(3-(2,4-dihydroxy-5-isopropylphenyl)-5-hydroxy-4H-1,2,4-triazol-4-yl)benzyl)piperidine-4-carboxylate

To a solution of Intermediate 5 (8.0 g, 17.07 mmol) in EtOH (80 mL) was added NH₂NH₂H₂O (1.11 g, 22.2 mmol). Then the resulting mixture was stirred at room temperature overnight. The precipitated solid was filtered and dried to give Intermediate 6 (4 g) as a white solid.

### Intermediate 7

### 1-(4-(3-(2,4-dihydroxy-5-isopropylphenyl)-5-hydroxy-4H-1,2,4-triazol-4-yl)benzyl)piperidine-4-carboxylic acid

To a solution of Intermediate 6 (4.0 g 8.57 mmol) in MeOH/THF (1:1, 30 mL) was added the solution (15 mL) of LiOH H₂O (1.8 g, 42.87 mmol). Then the resulting mixture was stirred at room temperature for 3 hours. The pH was adjusted to 5-6 with 2N HCl. The precipitated solid was filtered and dried to give Intermediate 7 (2.5 g) as a white solid.

### Intermediate 9

### (S)-tert-butyl 4-(4-((benzyloxy)carbonyl)-3-(cyanomethyl)piperazin-1-yl)-2-chloro-5,6-dihydropyrido[3,4-d]pyrimidine-7(8H)-carboxylate (2)

To a mixture of tert-butyl 2,4-dichloro-5,6-dihydropyrido[3,4-d]pyrimidine-7(8H)-carboxylate 8 (28 g, 92.4 mmol) and (S)-benzyl 2-(cyanomethyl)piperazine-1-carboxylate 8a (23.2 g, 89.5 mmol) in dioxane (300 mL) was added DIPEA (36.8 mL, 223 mmol). The mixture was stirred overnight under N₂ atmosphere at 50° C. The mixture was diluted with water (300 mL) and was extracted with ethyl acetate (300 mL×3). The combined organic phase was washed with brine (200 mL), dried over Na₂SO₄ and concentrated. The residue was purified by silica gel column chromatography (PE/EtOAc=5/1 to 1/1) to give the title Intermediate 9 (45 g, yield 90.7%) as a white solid. ¹H NMR (DMSO-d₆, 400 MHz), δ 7.39-7.33 (m, 5H), 5.17-5.11 (m, 2H), 4.58-4.26 (m, 3H), 4.05-3.92 (m, 3H), 3.66-3.63 (m, 1H), 3.31-2.90 (m, 6H), 2.69-2.68 (m, 2H), 1.43 (s, 9H); LC-MS: m/z 527.3 [M+H]⁺.

### Intermediate 10

### (S)-benzyl 4-(2-chloro-5,6,7,8-tetrahydropyrido[3,4-d]pyrimidin-4-yl)-2-(cyanomethyl)piperazine-1-carboxylate (3)

To a solution of (S)-tert-butyl 4-(4-((benzyloxy)carbonyl)-3-(cyanomethyl)piperazin-1-yl)-2-chloro-5,6-dihydropyrido[3,4-d]pyrimidine-7(8H)-carboxylate 9 (45 g, 85.5 mmol) in DCM (150 mL) was added TFA (50 mL). The mixture was stirred for 2 hours at room temperature and was concentrated under reduced pressure. The residue was diluted in EtOAc (500 mL) and sat.NaHCO₃ (500 mL). The organic phase was separated and washed with brine (500 mL), dried over Na₂SO₄ and concentrated to give the title Intermediate 10 (33 g, yield 90.6%) as a white solid. LC-MS: m/z 427.2[M+H]⁺.

### Intermediate 11

### (S)-benzyl 4-(2-chloro-7-(8-chloronaphthalen-1-yl)-5,6,7,8-tetra hydropyrido[3,4-d]pyrimidin-4-yl)-2-(cyanomethyl)piperazine-1-carboxylate

To a solution of (S)-benzyl 4-(2-chloro-5,6,7,8-tetrahydropyrido[3,4-d]pyrimidin-4-yl)-2-(cyanomethyl)piperazine-1-carboxylate 10 (25 g, 58.6 mmol) in toluene (200 mL) were added 1-bromo-8-chloronaphthalene (14.1 g, 58.6 mmol), Cs₂CO₃ (57.3 g, 176 mmol) and Ruphos Pd G3 (9.8 g, 11.7 mmol). The mixture was degassed for three times by N₂. Then the mixture was heated for 16 h at 75° C. After cooled to room temperature, the solid was filtered out. The filtrate was concentrated under vacuum. The residue was purified by silica gel column chromatography (PE:EtOAc=5:1 to 2:1) to give Intermediate 11 (5 g, yield 14.5%) as an off-white solid. ¹H NMR (CDCl₃, 400 MHz), δ 7.75 (d, J=8.4 Hz, 1H), 7.63 (t, J=7.6 Hz, 1H), 7.52 (d, J=6.0 Hz, 1H), 7.47-7.43 (m, 1H), 7.40-7.31 (m, 6H), 7.22-7.17 (m, 1H), 5.20 (s, 2H), 4.67 (s, 1H), 4.50-4.42 (m, 1H), 4.21-3.84 (m, 4H), 3.60-3.42 (m, 2H), 3.34-3.04 (m, 4H), 2.94-2.56 (m, 3H). LC-MS: m/z 587.2 [M+H]⁺.

### Intermediate 12

### (S)-benzyl 4-(2-(((R)-1-(tert-butoxycarbonyl)pyrrolidin-3-yl) methoxy)-7-(8-chloronaphthalen-1-yl)-5,6,7,8-tetrahydropyrido[3,4-d]pyrimidin-4-yl)-2-(cyanomethyl)piperazine-1-carboxylate

A mixture of Intermediate 11 (3.0 g, 5.11 mmol), Cs₂CO₃ (5.0 g, 15.3 mmol), (R)-tert-butyl 3-(hydroxymethyl)pyrrolidine-1-carboxylate (3.1 g, 15.3 mmol) and XPhos Pd G2 (600 mg, 0.77 mmol) in toluene (150 mL) was stirred at 85° C. for 16 h under N₂ atmosphere. The reaction was filtered and concentrated in vacuum to give a residue, which was purified by silica gel chromatography column (PE:EtOAc=4:1 to 2:1) to give Intermediate 12 (5.0 g, crude product) as a yellow oil. LC-MS: m/z 752.2 [M+H]⁺.

### Intermediate 13

### (R)-tert-butyl 3-(((7-(8-chloronaphthalen-1-yl)-4-((S)-3-(cyanomethyl) piperazin-1-yl)-5,6,7,8-tetrahydropyrido[3,4-d]pyrimidin-2-yl)oxy)methyl)pyrrolidine-1-carboxylate

A mixture of Intermediate 12 (5.0 g, 5.11 mmol, crude), ammonium formate (6.4 g, 102 mmol) and Pd/C (300 mg, 10% wet) in MeOH (100 mL) was refluxed for 0.5 h under N₂ atmosphere. The reaction was filtered and concentrated in vacuum to give a residue, which was purified by C18 flash chromatography column (ACN:H₂O=80:20 to 85:15) to give Intermediate 13 (1.4 g, 44.4% yield) as a white solid. LC-MS: m/z 618.1 [M+H]⁺.

### Intermediate 14

### Synthesis of (R)-tert-butyl 3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)pyrrolidine-1-carboxylate

To a solution of Intermediate 13 (1.4 g, 2.26 mmol) and DIPEA (730 mg, 5.66 mmol) in DCM (20 mL) was added acryloyl chloride (245 mg, 2.71 mmol) at 0° C., and the reaction was stirred at r.t for 1 h under N₂ atmosphere. The reaction was concentrated in vacuum to give a residue, which was purified by silica gel chromatography column (PE:EtOAc=3:2 to 2:3) to give Intermediate 14 (1.5 g, 98.8% yield) as a white solid. ¹H NMR (DMSO-d₆, 400 MHz): δ 7.92 (d, J=7.6 Hz, 1H), 7.75-7.72 (m, 1H), 7.75 (d, J=8.0 Hz, 1H), 7.58 (d, J=7.6 Hz, 1H), 7.53 (q, J=7.6 Hz, 1H), 7.45 (t, J=7.6 Hz, 1H), 7.33 (dd, J=15.2 Hz, 7.2 Hz, 1H), 6.85 (brs, 1H), 6.18 (d, J=28.8 Hz, 1H), 5.77 (d, J=12.4 Hz, 1H), 4.97-4.77 (m, 1H), 4.42-3.36 (m, 10H), 3.24-2.86 (m, 8H), 2.67-2.51 (m, 2H), 1.96 (brs, 1H), 1.66 (brs, 1H), 1.38 (s, 9H). LC-MS: m/z 672.1 [M+H]⁺.

### Intermediate 15

### 2-((S)-1-acryloyl-4-(7-(8-chloronaphthalen-1-yl)-2-((R)-pyrrolidin-3-ylmethoxy)-5,6,7,8-tetrahydropyrido[3,4-d]pyrimidin-4-yl)piperazin-2-yl)acetonitrile

To a solution of Intermediate 14 (600 mg, 0.89 mmol) in DCM (3 mL) was added TFA (1 mL) at 0° C., and the reaction was stirred at r.t for 1 h under N₂ atmosphere. The reaction was concentrated in vacuum to give the crude Intermediate 15 (550 mg) as a yellow oil, which was used directly for next step. LC-MS: m/z 572.5 [M+H]⁺.

### Compound 148

### 2-((S)-1-acryloyl-4-(7-(8-chloronaphthalen-1-yl)-2-(((R)-1-(1-(4-(3-(2,4-dihydroxy-5-isopropylphenyl)-5-hydroxy-4H-1,2,4-triazol-4-yl)benzyl)piperidine-4-carbonyl)pyrrolidin-3-yl)methoxy)-5,6,7,8-tetrahydropyrido[3,4-d]pyrimidin-4-yl)piperazin-2-yl)acetonitrile

A mixture of compound 5 (600 mg, 0.87 mmol, crude), Intermediate 15 (453 mg, 0.96 mmol), PyBOP (545 mg, 1.05 mmol) and DIPEA (338 mg, 2.62 mmol) in DMF (8 mL) was stirred at r.t for 16 h. The mixture was purified by pre-HPLC (TFA) to give Compound 148 (260 mg, 29.6% yield) as a white solid. ¹H NMR (DMSO-d₆, 400 MHz): δ 11.93 (s, 1H), 9.59 (s, 1H), 9.42 (s, 1H), 7.92 (d, J=8.0 Hz, 1H), 7.74 (d, J=8.0 Hz, 1H), 7.58 (d, J=6.8 Hz, 1H), 7.53 (q, J=7.6 Hz, 1H), 7.45 (t, J=7.6 Hz, 1H), 7.36-7.27 (m, 3H), 7.12 (d, J=8.0 Hz, 2H), 6.84 (brs, 1H), 6.75 (s, 1H), 6.26 (s, 1H), 6.18 (d, J=16.8 Hz, 1H), 5.77 (d, J=10.4 Hz, 1H), 4.96-4.73 (m, 1H), 4.41-4.12 (m, 4H), 4.02-3.40 (m, 10H), 3.28-2.54 (m, 14H), 2.38-2.31 (m, 2H), 1.64-1.51 (m, 4H), 0.92 (dd, J=6.8 Hz, 2.0 Hz, 6H). LC-MS: m/z 1006.4 [M+H]⁺.

### Example 2: Compound 175 Synthesis

A representative synthesis scheme for compound 175 is shown in below. Specific synthesis routes of intermediates are also shown.

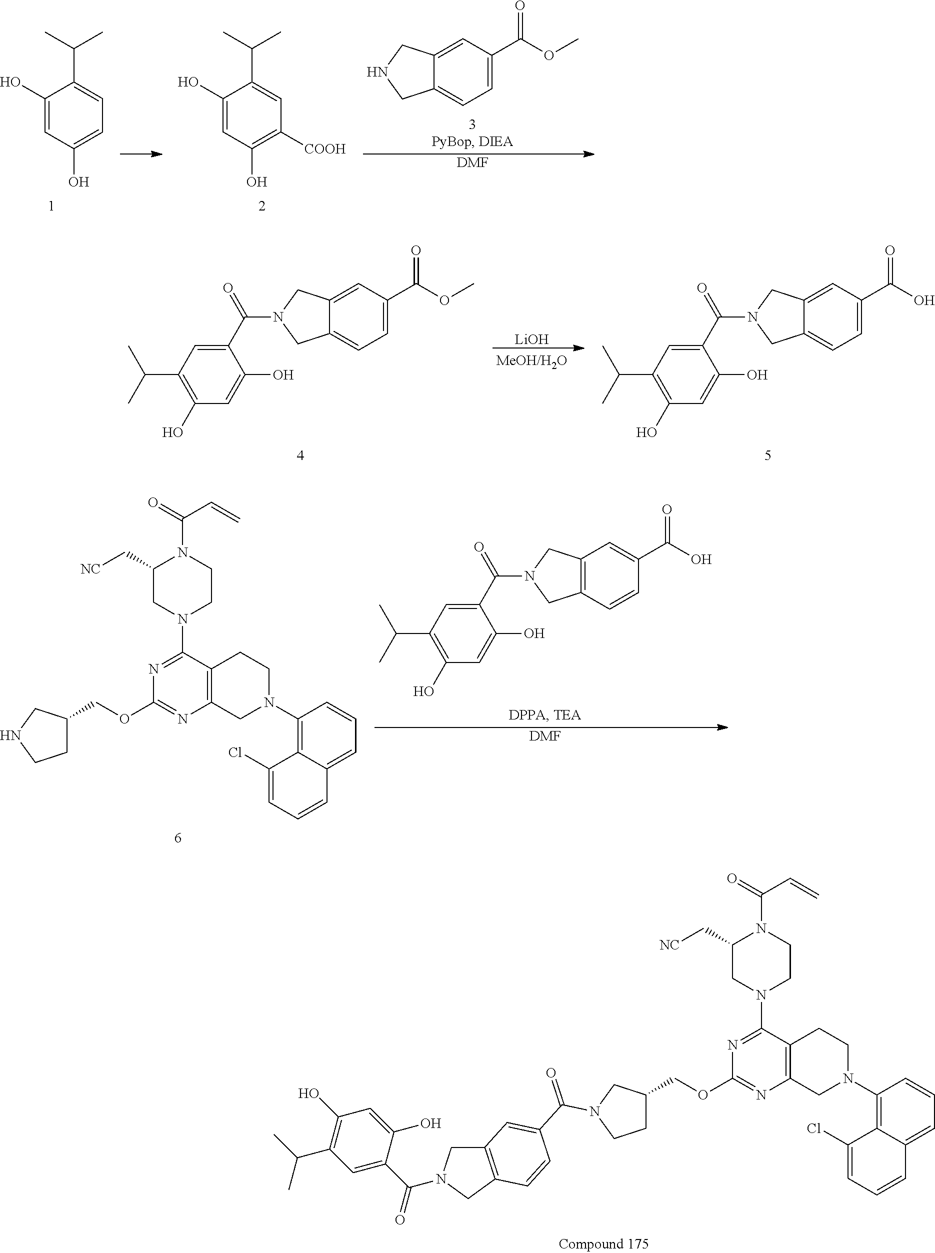

### Intermediate 2

To a solution of 4-isopropylbenzene-1,3-diol (100 g, 658 mmol) in H₂O (1 L) was added KHCO₃ (493 g, 4.9 mol). The resulting mixture was stirred at reflux overnight while CO₂ bubbling in. The reaction solution was cooled to room temperature and 2N HCl was added slowly to adjust the pH to 1. The solid precipitated was collected by filtration and dried to give target compound (70 g, 54% yield) as a white solid.

### Intermediate 4

### methyl 2-(2,4-dihydroxy-5-isopropylbenzoyl)isoindoline-5-carboxylate

A mixture of 2,4-dihydroxy-5-isopropylbenzoic acid (254 mg, 1.3 mmol), methyl isoindoline-5-carboxylate (191 mg, 1.08 mmol), PyBOP (1.1 g, 2.2 mmol) and DIPEA (557 mg, 4.3 mmol) in DMF (10 mL) was stirred at r.t for 3 h. The mixture was poured into H₂O (40 mL). The precipitate was filtered and purified by pre-TLC (EA) to give methyl 2-(2,4-dihydroxy-5-isopropylbenzoyl)isoindoline-5-carboxylate (301 mg, 78% yield) as a red solid.

¹H NMR (DMSO-d₆, 400 MHz): δ 10.06-10.02 (m, 1H), 9.61 (s, 1H), 7.95-7.88 (m, 2H), 7.50-7.44 (m, 1H), 7.05 (s, 1H), 6.40 (s, 1H), 4.84 (s, 4H), 3.85 (s, 3H), 3.13-3.06 (m, 1H), 1.13 (d, J=6.8 Hz, 6H). LC-MS: m/z 356.1 [M+H]⁺.

### Intermediate 5

### 2-(2,4-dihydroxy-5-isopropylbenzoyl)isoindoline-5-carboxylic acid

A mixture of methyl 2-(2,4-dihydroxy-5-isopropylbenzoyl)isoindoline-5-carboxylate (301 mg, 0.85 mmol) and LiOH·H₂O (110 mg, 2.6 mmol) in MeOH/H₂O (50 mL/10 mL) was stirred at r.t. for 24 h. The reaction was concentrated in vacuum to give a residue, which was adjusted PH to 4. The precipitate was filtered and triturated with a mixture of THF (20 mL) and PE (150 mL) to give 2-(2,4-dihydroxy-5-isopropylbenzoyl)isoindoline-5-carboxylic acid (210 mg, 72% yield) as a white solid.

¹H NMR (DMSO-d₆, 400 MHz): δ 12.94 (brs, 1H), 10.04-10.02 (m, 1H), 9.62 (s, 1H), 7.95-7.84 (m, 2H), 7.49-7.41 (m, 1H), 7.04 (s, 1H), 6.40 (s, 1H), 4.82 (s, 4H), 3.12-3.05 (m, 1H), 1.13 (d, J=6.4 Hz, 6H). LC-MS: m/z 342.0 [M+H]⁺.

### Compound 175

### 2-((S)-1-acryloyl-4-(7-(8-chloronaphthalen-1-yl)-2-(((R)-1-(2-(2,4-dihydroxy-5-isopropylbenzoyl)isoindoline-5-carbonyl)pyrrolidin-3-yl)methoxy)-5,6,7,8-tetrahydropyrido[3,4-d]pyrimidin-4-yl)piperazin-2-yl)acetonitrile

A mixture of 2-((S)-1-acryloyl-4-(7-(8-chloronaphthalen-1-yl)-2-((R)-pyrrolidin-3-ylmethoxy)-5,6,7,8-tetrahydropyrido[3,4-d]pyrimidin-4-yl)piperazin-2-yl)acetonitrile (63 mg, 0.11 mmol), 2-(2,4-dihydroxy-5-isopropylbenzoyl)isoindoline-5-carboxylic acid (35 mg, 0.11 mmol), DPPA (71 mg, 0.26 mmol) and TEA (42 mg, 0.42 mmol) in DMF (4 mL) was stirred at r.t for 17 h. The mixture was poured into H₂O (15 mL). The precipitate was filtered and purified by pre-HPLC (TFA) to give Compound 175

(5.1 mg, 5.5% yield) as a white solid.

¹H NMR (CD₃OD, 400 MHz): δ 7.87 (d, J=8.4 Hz, 1H), 7.75 (dd, J=8.0, 4.0 Hz, 1H), 7.60-7.36 (m, 7H), 7.18 (d, J=5.6 Hz, 1H), 6.82-6.80 (m, 1H), 6.38 (s, 1H), 6.32 (d, J=16.4 Hz, 1H), 5.86 (d, J=10.4 Hz, 1H), 4.96 (s, 4H), 4.76-4.29 (m, 6H), 4.18-4.16 (m, 1H), 3.88-3.37 (m, 9H), 3.27-3.15 (m, 2H), 3.08-2.79 (m, 4H), 2.29-2.16 (m, 1H), 2.01-1.90 (m, 1H), 1.22 (d, J=7.2 Hz, 3H), 1.20 (d, J=7.2 Hz, 3H). LC-MS: m/z 895.4 [M+H]⁺.

### Example 3: Compound 178 Synthesis

A representative synthesis scheme for compound 178 is shown in below. Specific synthesis routes of intermediates are also shown.

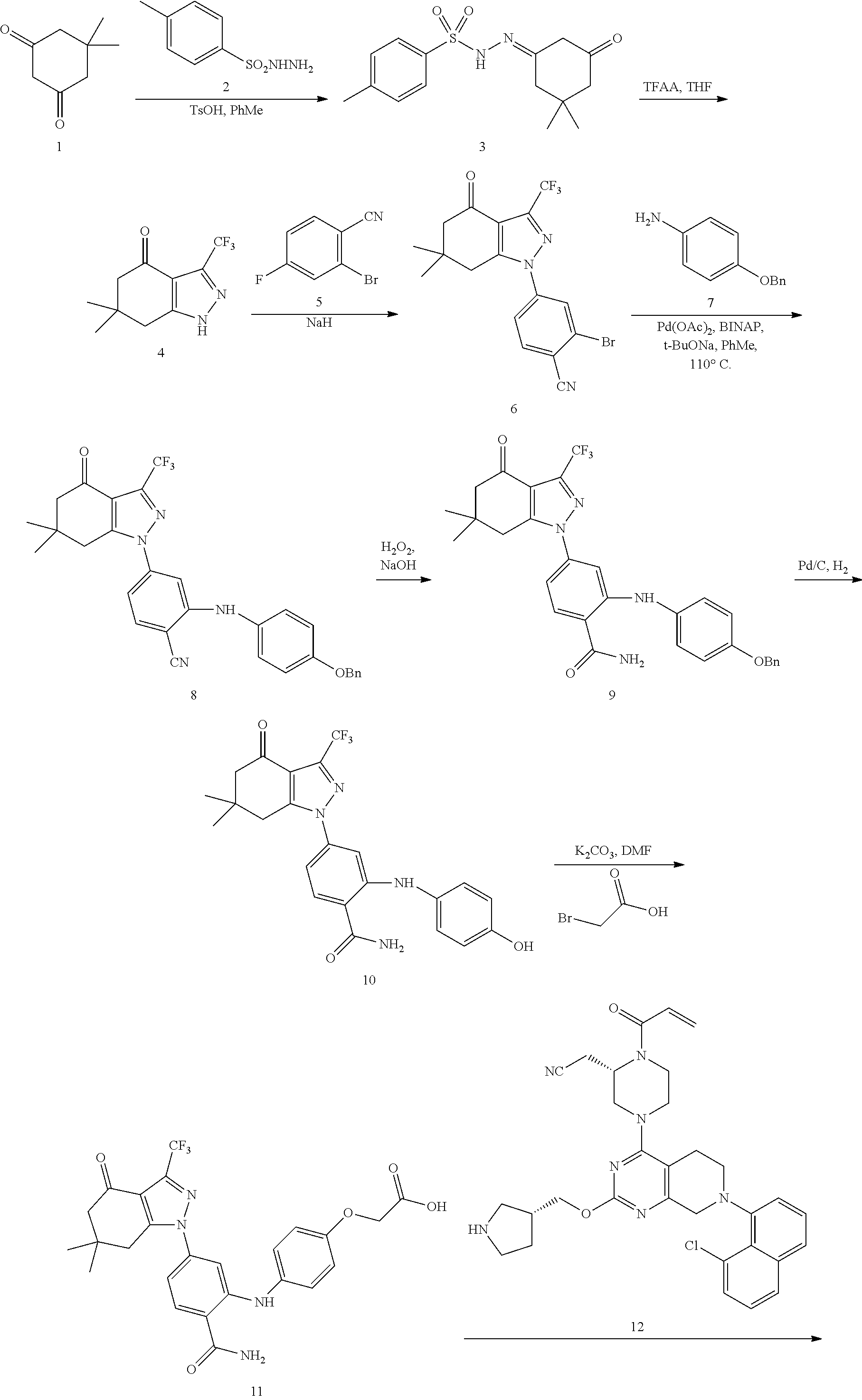

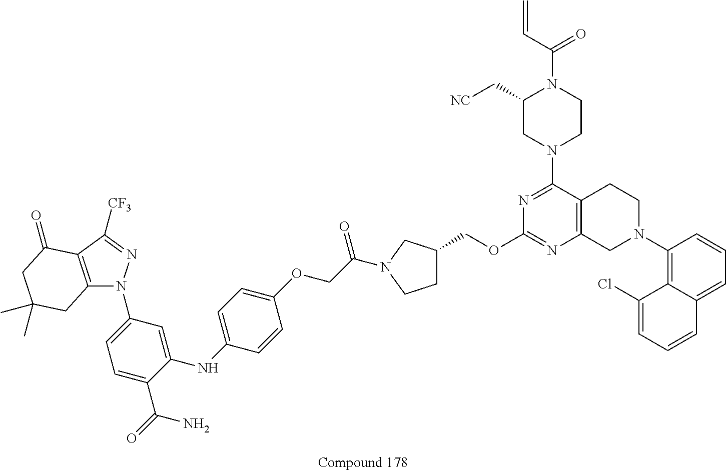 [SMILES: C=CC(=O)N1CCN(c2nc(OC[C@@H]3CCN(C(=O)COc4ccc(Nc5cc(-n6nc(C(F)(F)F)c7c6CC(C)(C)CC7=O)ccc5C(N)=O)cc4)C3)nc3c2CCN(c2cccc4cccc(Cl)c24)C3)C[C@@H]1CC#N]

### Intermediate 3

### (E)-N′-(3,3-dimethyl-5-oxocyclohexylidene)-4-methylbenzenesulfonohydrazide

A mixture of 1 (200 g, 1426.72 mmol), 2 (265.71 g, 1426.7167 mmol) and p-Toluene sulfonic acid (24.54 g, 142.67 mmol) in toluene (8 L) was heated to 120° C. After 1 h, the mixture was cooled and followed by the addition of toluene (1.2 L). The mixture was then reflux for 1 h. The reaction was cooled to ambient temperature. The precipitated solids was collected by filtration, washed three times with ether and dried under vacuum to give intermediate 3 (360 g, 1167.30 mmol, 81.82%). LCMS: m/z 309 [M+H]⁺.

### Intermediate 4

### 6,6-dimethyl-3-(trifluoromethyl)-1,5,6,7-tetrahydro-4H-indazol-4-one

To a suspension of 3 (360 g, 1167.30 mmol) and TEA (486.67 mL, 3501.33 mmol) in THF (3 L) was added trifluoroacetyl 2,2,2-trifluoroacetate (243.51 mL, 1750.67 mmol) at 0° C. The resulting reaction was heated to 55° C. for 3 h, the reaction mixture was cooled to ambient temperature. To the mixture was added Methanol (1.4 L) and 1 N NaOH (1.4 L). After stirring for 3 h, the reaction mixture was diluted with saturated ammonium chloride (3 L), extracted with ethyl acetate three times, the combine organic layers was washed with brine, dried over sodium sulfate, and concentrated in vacuum. The residue was purified by column chromatography to give the intermediate 4 (160 g, 689.05 mmol, 59.04%). LCMS: m/z 233 [M+H]⁺.

### Intermediate 6

### 2-bromo-4-(6,6-dimethyl-4-oxo-3-(trifluoromethyl)-4,5,6,7-tetrahydro-1H-indazol-1-yl)benzonitrile

NaH (15.50 g, 645.98 mmol) was added to a solution of 4 (150 g, 645.98 mmol) in DMSO (2 L) at room temperature. After 15 min, 2-bromo-4-fluorobenzonitrile (129.20 g, 645.98 mmol) was added as solid. The reaction mixture was heated at 45° C. overnight. The mixture was cooled to room temperature and quenched with saturated aqueous NH₄Cl. The mixture was diluted with water and extracted with EtOAc. The combined organic layers were washed with brine, dried over anhydrous sodium sulfate and concentrated in vacuum. The residue was purified by column chromatography to give intermediate 6 (180 g, 436.67 mmol, 67.59%). LCMS: m/z 412 [M+H]⁺.

### Intermediate 8

### 2-((4-(benzyloxy)phenyl)amino)-4-(6,6-dimethyl-4-oxo-3-(trifluoromethyl)-4,5,6,7-tetrahydro-1H-indazol-1-yl)benzonitrile

To a solution of 6 (50 g, 121.30 mmol) in toluene (500 mL) was added 7 (24.17 g, 121.30 mmol) and Cs₂CO₃ (79.04 g, 242.59 mmol). Then BINAP (15.10 g, 24.26 mmol) and Pd(OAc)₂ (2.74 g, 12.13 mmol) was added successively under nitrogen protection. The mixture reaction was heated to 120° C. for 3 h. After which in was filtered, the filtrate was concentrated in vacuum, the residue was purified by silica gel chromatography to give the intermediate 8 (40 g, 75.39 mmol, 62.16%). LCMS: m/z 531 [M+H]⁺.

### Intermediate 9

### 2-((4-(benzyloxy)phenyl)amino)-4-(6,6-dimethyl-4-oxo-3-(trifluoromethyl)-4,5,6,7-tetrahydro-1H-indazol-1-yl)benzamide

To a solution of 8 (40 g, 75.39 mmol) in EtOH (400 mL) and DMSO (100 mL) was added 1 N NaOH (226.18 mL, 226.18 mmol) and H₂O₂ (25.63 g, 226.18 mmol) dropwise successively at 0° C. Then the mixture was stirred at RT for 2 h before diluting with water, it was extracted with EtOAc, washed with brine, dried over sodium sulfate. The organic layer was concentrated in vacuum, the residue was purified by silica gel column to give the intermediate 9 (35 g, 63.80 mmol, 84.63%). LCMS: m/z 549 [M+H]⁺.

### Intermediate 10

### 4-(6,6-dimethyl-4-oxo-3-(trifluoromethyl)-4,5,6,7-tetrahydro-1H-indazol-1-yl)-2-((4-hydroxyphenyl)amino)benzamide

To a solution of 9 (35 g, 63.80 mmol) in MeOH (400 mL) was added Pd/C 10% (6.7 g, 6.38 mmol), the mixture was stirred at RT overnight with H₂ existence. After which it was filtered, washed with EA followed by DCM, the filler was concentrated in vacuum to give intermediate 10 (26 g, 56.71 mmol, 88.89%) as a solid. LCMS: m/z 459 [M+H]⁺.

### Intermediate 11

### 2-(4-((2-carbamoyl-5-(6,6-dimethyl-4-oxo-3-(trifluoromethyl)-4,5,6,7-tetrahydro-1H-indazol-1-yl)phenyl)amino)phenoxy)acetic acid

To a solution of 2-bromoacetic acid (0.6 g, 4.59 mmol) in DMF (20 mL) was added 10 (1.89 g, 4.13 mmol) and K₂CO₃ (1.90 g, 13.76 mmol), the mixture was stirred at 90° C. overnight. Then water was added, it was extracted with EA, washed with saturated brine, dried over sodium sulfate, concentrated in vacuum, the residue was purified by silica gel column to give the intermediate (1.6 g, 2.48 mmol, 54.01%) as a solid. LCMS: 518 [M+H]⁺.

### Compound 178

### 2-((4-(2-((R)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)pyrrolidin-1-yl)-2-oxoethoxy)phenyl)amino)-4-(6,6-dimethyl-4-oxo-3-(trifluoromethyl)-4,5,6,7-tetrahydro-1H-indazol-1-yl)benzamide

To a solution of intermediate 11 (340 mg, 0.7 mmol), HATU (290 mg, 0.77 mmol) and DIEA (450 mg, 3.48 mmol) in DMF (8 mL) was added intermediate 12 (350 mg, 0.7 mmol).

The resulting mixture was stirred at room temperature for 2 hours. The mixture was purified by prep-HPLC to give compound 178 (230 mg) as yellow solid. H NMR (DMSO-d₆, 400 MHz): δ 10.09 (s, 1H), 8.19 (s, 1H), 7.92-7.88 (m, 2H), 7.74 (d, J=8.0 Hz, 1H), 7.58-7.53 (m, 3H), 7.50 (t, J=7.2 Hz, 1H), 7.44-7.30 (m, 1H), 7.20 (brs, 2H), 7.03 (s, 1H), 6.95-6.82 (m, 4H), 6.18 (d, J=16.4 Hz, 1H), 5.77 (d, J=10.8 Hz, 1H), 4.96-4.75 (m, 1H), 4.71 (s, 2H), 4.41-4.14 (m, 4H), 4.03-3.48 (m, 9H), 3.24-3.15 (m, 5H), 3.09 (s, 3H), 3.02-2.54 (m, 2H), 2.40 (d, J=5.6 Hz, 2H), 2.12-1.61 (m, 3H), 1.02 (s, 6H). LC-MS: m/z 1070.4 [M+H]⁺.

Additional compounds were made according to the general procedure and scheme noted in the Examples including the following:

### Compound 1

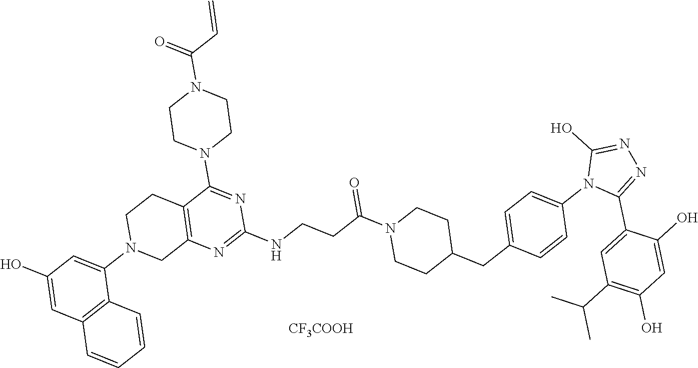 [SMILES: C=CC(=O)N1CCN(c2nc(NCCC(=O)N3CCC(Cc4ccc(-n5c(O)nnc5-c5cc(C(C)C)c(O)cc5O)cc4)CC3)nc3c2CCN(c2cc(O)cc4ccccc24)C3)CC1 ; "CF3COOH"]

### 1-(4-(2-((3-(4-(4-(3-(2,4-dihydroxy-5-isopropylphenyl)-5-hydroxy-4H-1,2,4-triazol-4-yl)benzyl)piperidin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 11.94 (s, 1H), 9.76 (s, 1H), 9.63 (s, 1H), 9.44 (s, 1H), 7.99 (d, J=8.4 Hz, 1H), 7.68 (d, J=8.4 Hz, 1H), 7.42-7.08 (m, 6H), 6.92-6.70 (m, 4H), 6.30-6.08 (m, 2H), 5.77-5.71 (m, 1H), 4.42-4.32 (m, 1H), 4.12 (s, 1H), 3.90-3.50 (m, 15H), 3.27-3.19 (m, 2H), 3.00-2.86 (m, 3H), 2.70-2.59 (m, 2H), 1.80-1.50 (m, 3H), 1.29-1.20 (m, 2H), 0.92 (d, J=6.8 Hz, 6H). LCMS (ESI): m/z found 893.5[M−CF3COOH+H]⁺.

### Compound 2

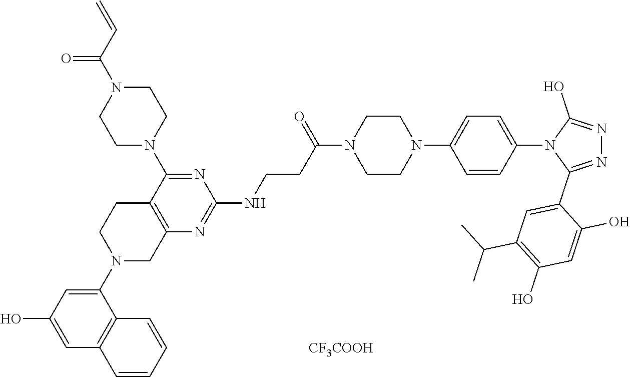 [SMILES: C=CC(=O)N1CCN(c2nc(NCCC(=O)N3CCN(c4ccc(-n5c(O)nnc5-c5cc(C(C)C)c(O)cc5O)cc4)CC3)nc3c2CCN(c2cc(O)cc4ccccc24)C3)CC1 ; "CF3COOH"]

### 1-(4-(2-((3-(4-(4-(3-(2,4-dihydroxy-5-isopropylphenyl)-5-hydroxy-4H-1,2,4-triazol-4-yl)phenyl)piperazin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 11.87 (s, 1H), 9.90-9.35 (m, 3H), 7.99 (d, J=8.4 Hz, 1H), 7.85-7.61 (m, 2H), 7.44-7.24 (m, 2H), 7.07-6.75 (m, 8H), 6.31-6.09 (m, 2H), 5.82-5.68 (m, 1H), 4.17 (s, 2H), 3.91-3.57 (m, 14H), 3.27-3.10 (m, 6H), 3.03-2.88 (m, 3H), 2.78-2.67 (m, 2H), 0.96 (d, J=7.2 Hz, 6H). LCMS (ESI): m/z found 880.4[M−CF3COOH+H]⁺

### Compound 3

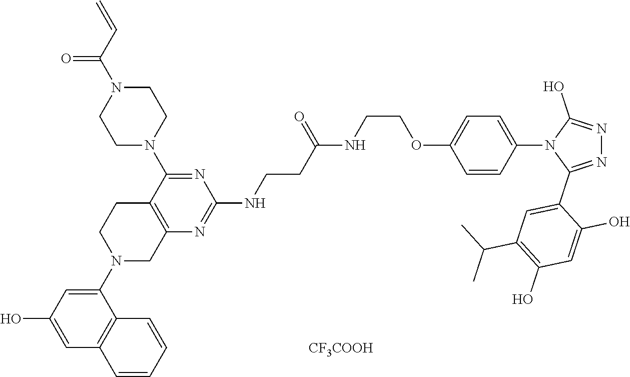 [SMILES: C=CC(=O)N1CCN(c2nc(NCCC(=O)NCCOc3ccc(-n4c(O)nnc4-c4cc(C(C)C)c(O)cc4O)cc3)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(2-(4-(3-(2,4-dihydroxy-5-isopropylphenyl)-5-hydroxy-4H-1,2,4-triazol-4-yl)phenoxy)ethyl)propanamide, trifluoroacetic acid

¹H NMR (400 MHz, DMSO-d₆): δ 11.89 (s, 1H), 9.90-9.35 (m, 3H), 8.31-8.23 (m, 1H), 8.06-7.62 (m, 3H), 7.44-7.24 (m, 2H), 7.11-6.74 (m, 8H), 6.29-6.14 (m, 2H), 5.79-5.69 (m, 1H), 4.16 (s, 2H), 4.00-3.56 (m, 14H), 3.27-3.21 (m, 2H), 3.03-2.86 (m, 3H), 2.48-2.44 (m, 2H), 0.97 (d, J=7.2 Hz, 6H). LCMS (ESI): m/z found 855.4[M—CF3COOH+H]⁺.

### Compound 4

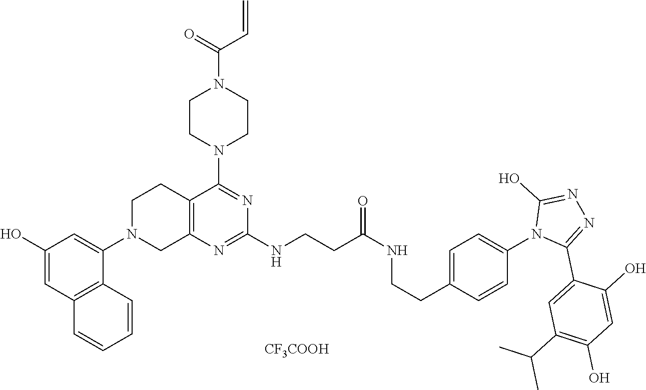 [SMILES: C=CC(=O)N1CCN(c2nc(NCCC(=O)NCCc3ccc(-n4c(O)nnc4-c4cc(C(C)C)c(O)cc4O)cc3)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)phenethyl)propanamide, trifluoroacetic acid

¹H NMR (400 MHz, DMSO-d₆): δ 11.91 (s, 1H), 9.76-9.38 m, 3H), 8.18-7.94 (m, 2H), 7.68 (d, J=8.4 Hz, 1H), 7.43-7.07 (m, 6H), 6.94-6.72 (m, 4H), 6.28-6.12 (m, 2H), 5.74 (dd, J=10.4, 2.2 Hz, 1H), 4.15 (s, 2H), 3.92-3.53 (m, 10H), 3.03-2.87 (m, 3H), 2.74-2.65 (m, 2H), 2.46-2.39 (m, 3H), 2.08-1.93 (m, 1H), 1.24 (s, 3H), 0.96 (d, J=6.8 Hz, 6H). LCMS (ESI): m/z found 839.4[M−CF3COOH+H]⁺.

### Compound 5

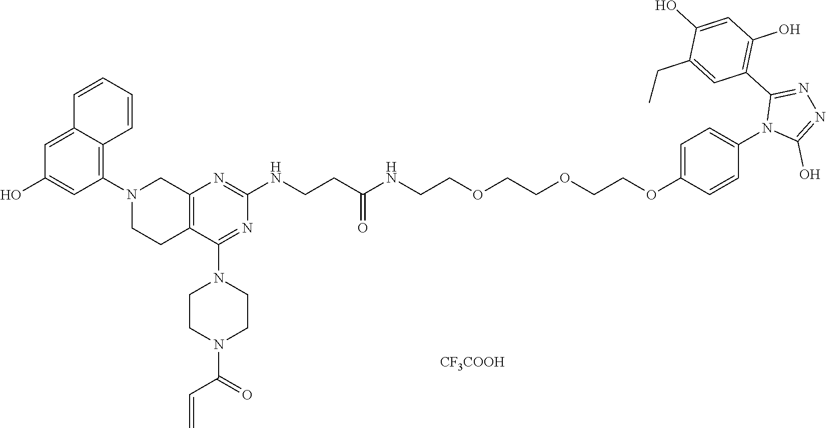 [SMILES: C=CC(=O)N1CCN(c2nc(NCCC(=O)NCCOCCOCCOc3ccc(-n4c(O)nnc4-c4cc(CC)c(O)cc4O)cc3)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(2-(2-(2-(4-(3-(5-ethyl-2,4-dihydroxyphenyl)-5-hydroxy-4H-1,2,4-triazol-4-yl)phenoxy)ethoxy)ethoxy)ethyl)propanamide, trifluoroacetic acid

¹H NMR (400 MHz, CD₃OD) δ 8.04 (d, J=8.4 Hz, 1H), 7.63 (d, J=8.0 Hz, 1H), 7.33 (dt, J=15.0, 7.2 Hz, 2H), 7.12 (d, J=8.8 Hz, 2H), 6.98-6.66 (m, 6H), 6.33-6.21 (m, 2H), 5.81 (d, J=9.6 Hz, 1H), 5.11-4.92 (m, 5H), 4.20-3.31 (m, 27H), 3.00-2.85 (m, 2H), 2.54 (t, J=6.2 Hz, 2H), 2.34 (q, J=7.5 Hz, 2H), 0.94 (t, J=7.6 Hz, 3H). LCMS (ESI): m/z found 929.2[M−CF3COOH+H]⁺.

### Compound 6

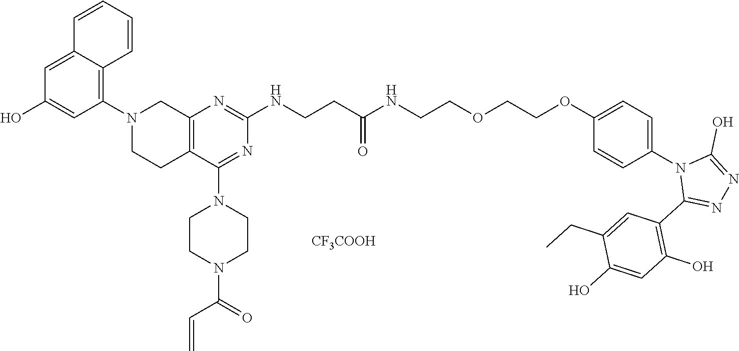 [SMILES: C=CC(=O)N1CCN(c2nc(NCCC(=O)NCCOCCOc3ccc(-n4c(O)nnc4-c4cc(CC)c(O)cc4O)cc3)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(2-(2-(4-(3-(5-ethyl-2,4-dihydroxyphenyl)-5-hydroxy-4H-1,2,4-triazol-4-yl)phenoxy)ethoxy)ethyl)propanamide, trifluoroacetic acid

¹H NMR (400 MHz, DMSO-d₆): δ 11.85 (s, 1H), 9.76 (s, 1H), 9.56 (s, 1H), 9.34 (s, 1H), 8.16-7.93 (m, 2H), 7.68 (d, J=8.2 Hz, 1H), 7.42-7.26 (m, 2H), 7.14-6.70 (m, 9H), 6.30-6.11 (m, 2H), 5.74 (d, J=12.6 Hz, 1H), 4.18-3.68 (m, 15H), 3.29-3.20 (m, 5H), 3.01-2.84 (m, 2H), 2.49-2.30 (m, 6H), 0.99 (t, J=7.6 Hz, 3H). LCMS (ESI): found 885.4[M−CF3COOH+H]⁺.

### Compound 7

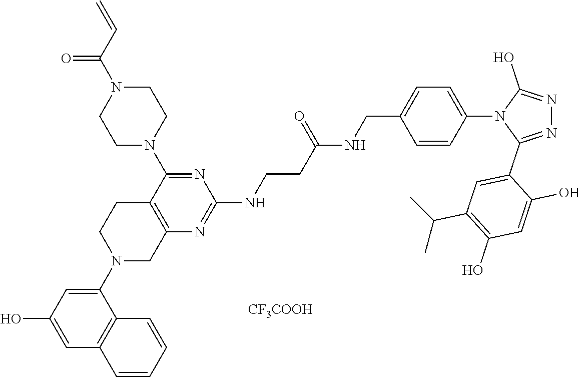 [SMILES: C=CC(=O)N1CCN(c2nc(NCCC(=O)NCc3ccc(-n4c(O)nnc4-c4cc(C(C)C)c(O)cc4O)cc3)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)benzyl)propanamide, trifluoroacetic acid

¹H NMR (400 MHz, DMSO-d₆) δ 11.91 (s, 1H), 9.79 (s, 1H), 9.60 (s, 1H), 9.37 (s, 1H), 8.52 (t, J=5.6 Hz, 1H), 8.03-7.66 (m, 3H), 7.41 (t, J=7.5 Hz, 1H), 7.29 (t, J=7.4 Hz, 1H), 7.23 (d, J=8.3 Hz, 2H), 7.11 (d, J=8.3 Hz, 2H), 6.90 (s, 1H), 6.84 (s, 1H), 6.82-6.74 (m, 2H), 6.24 (s, 1H), 6.17 (m, 1H), 5.74 (m, 2.0 Hz, 1H), 4.27 (d, J=5.7 Hz, 2H), 4.16 (s, 2H), 3.87 (s, 4H), 3.79-3.59 (m, 7H), 3.25 (s, 3H), 2.97 (m, 3H), 0.99 (d, J=6.9 Hz, 6H). LCMS (ESI): m/z found 825.6 [M−CF3COOH+H]⁺.

### Compound 8

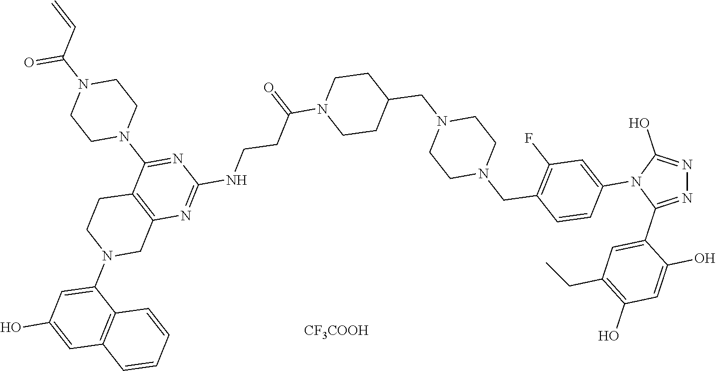 [SMILES: C=CC(=O)N1CCN(c2nc(NCCC(=O)N3CCC(CN4CCN(Cc5ccc(-n6c(O)nnc6-c6cc(CC)c(O)cc6O)cc5F)CC4)CC3)nc3c2CCN(c2cc(O)cc4ccccc24)C3)CC1 ; "CF3COOH"]

### 1-(4-(2-((3-(4-((4-(4-(3-(5-ethyl-2,4-dihydroxyphenyl)-5-hydroxy-4H-1,2,4-triazol-4-yl)-2-fluorobenzyl)piperazin-1-yl)methyl)piperidin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆) δ 11.99 (s, 1H), 9.77 (s, 1H), 9.62 (s, 1H), 9.38 (s, 1H), 8.06-7.93 (m, 1H), 7.69 (d, J=8.0 Hz, 1H), 7.35 (m, 3H), 7.16-6.68 (m, 6H), 6.30-6.10 (m, 2H), 5.75 (d, J=12.5 Hz, 1H), 4.38 (m, 1H), 4.16 (s, 2H), 3.89 (m, 17H), 3.25 (s, 2H), 3.11-2.83 (m, 8H), 2.79-2.55 (m, 6H), 2.45-2.28 (m, 3H), 2.01 (s, 1H), 1.73 (m, 2H), 1.02 (t, J=7.5 Hz, 3H). LCMS (ESI): S m/z found 995.3 [M—CF3COOH+H]⁺.

### Compound 9

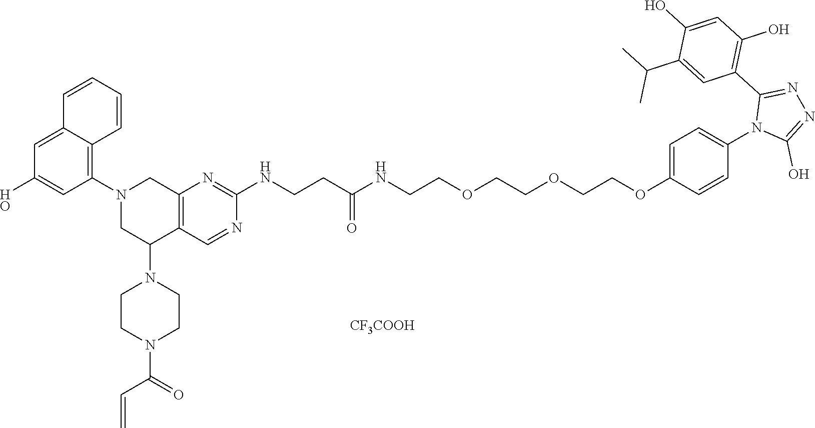 [SMILES: C=CC(=O)N1CCN(C2CN(c3cc(O)cc4ccccc34)Cc3nc(NCCC(=O)NCCOCCOCCOc4ccc(-n5c(O)nnc5-c5cc(C(C)C)c(O)cc5O)cc4)ncc32)CC1 ; "CF3COOH"]

### 3-((5-(4-acryloylpiperazin-1-yl)-7-(3-hydroxynaphthalen-1-yl)-5,6,7,8-tetrahydropyrido[3,4-d]pyrimidin-2-yl)amino)-N-(2-(2-(2-(4-(3-(2,4-dihydroxy-5-isopropylphenyl)-5-hydroxy-4H-1,2,4-triazol-4-yl)phenoxy)ethoxy)ethoxy)ethyl)propanamide, trifluoroacetic acid

¹H NMR (400 MHz, DMSO-d₆) δ 11.88 (s, 1H), 9.80 (s, 1H), 9.62 (s, 1H), 9.40 (s, 1H), 8.07 (s, 1H), 7.99 (d, J=8.4 Hz, 1H), 7.88 (s, 1H), 7.68 (d, J=8.2 Hz, 1H), 7.40 (t, J=7.3 Hz, 1H), 7.28 (t, J=7.3 Hz, 1H), 7.09 (d, J=8.9 Hz, 2H), 6.91 (d, J=9.1 Hz, 3H), 6.85-6.75 (m, 3H), 6.26 (s, 1H), 6.17 (dd, J=16.7, 2.1 Hz, 1H), 5.74 (dd, J=10.5, 2.0 Hz, 1H), 4.15 (s, 2H), 4.08-4.03 (m, 2H), 3.88 (s, 4H), 3.73 (m, 7H), 3.60-3.52 (m, 7H), 3.22 (m, 5.5 Hz, 4H), 2.97 (m, 3H), 2.44 (t, J=6.6 Hz, 2H), 0.98 (d, J=6.9 Hz, 6H). LCMS (ESI): m/z found 943.6 [M—CF3COOH+H]⁺.

### Compound 10

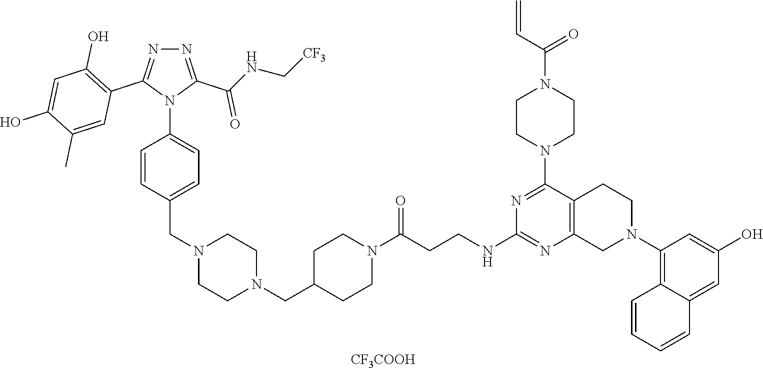 [SMILES: C=CC(=O)N1CCN(c2nc(NCCC(=O)N3CCC(CN4CCN(Cc5ccc(-n6c(C(=O)NCC(F)(F)F)nnc6-c6cc(C)c(O)cc6O)cc5)CC4)CC3)nc3c2CCN(c2cc(O)cc4ccccc24)C3)CC1 ; "CF3COOH"]

### 4-(4-((4-((1-(3-((4-(4-acryloylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(2,4-dihydroxy-5-methylphenyl)-N-(2,2,2-trifluoroethyl)-4H-1,2,4-triazole-3-carboxamide, trifluoroacetic acid

¹H NMR (400 MHz, DMSO-d₆): δ 9.85-9.67 (m, 4H), 7.97 (d, J=5.2 Hz, 1H), 7.68 (d, J=5.2 Hz, 1H), 7.41-7.28 (m, 6H), 6.90-6.74 (m, 4H), 6.28-6.15 (m, 2H), 5.78-5.72 (m, 1H), 4.44-4.38 (m, 1H), 4.16 (s, 3H), 3.94-3.80 (m, 10H), 3.66-3.60 (m, 4H), 3.31-3.24 (m, 4H), 3.09-2.85 (m, 9H), 2.67-2.58 (s, 4H), 2.42-2.32 (m, 6H), 1.87 (s, 3H), 1.75-1.69 (m, 2H). LCMS (ESI): m/z found 1073.1[M−CF3COOH+H]⁺

### Compound 11

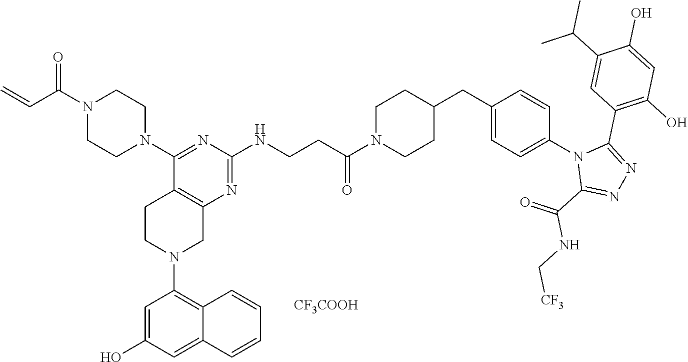 [SMILES: C=CC(=O)N1CCN(c2nc(NCCC(=O)N3CCC(Cc4ccc(-n5c(C(=O)NCC(F)(F)F)nnc5-c5cc(C(C)C)c(O)cc5O)cc4)CC3)nc3c2CCN(c2cc(O)cc4ccccc24)C3)CC1 ; "CF3COOH"]

### 4-(4-((1-(3-((4-(4-acryloylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆): δ 9.78-9.60 (m, 3H), 8.00-7.98 (m, 1H), 7.69 (d, J=8.8 Hz, 1H), 7.42-7.38 (m, 1H), 7.30-7.23 (m, 6H), 6.89-6.75 (m, 3H), 6.61 (s, 1H), 6.35 (s, 1H), 4.43-4.35 (m, 2H), 4.18-4.15 (m, 3H), 3.95-3.68 (m, 18H), 3.27-3.22 (m, 7H), 2.94-2.89 (m, 5H), 0.82 (d, J=6.8 Hz, 6H). LCMS (ESI): m/z found 1003.0[M−CF3COOH+H]⁺.

### Compound 12

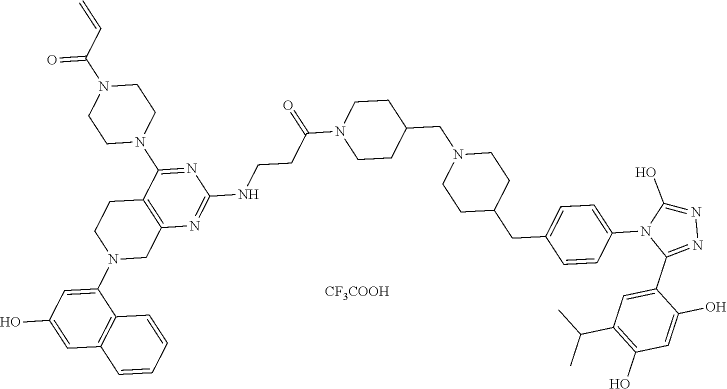 [SMILES: C=CC(=O)N1CCN(c2nc(NCCC(=O)N3CCC(CN4CCC(Cc5ccc(-n6c(O)nnc6-c6cc(C(C)C)c(O)cc6O)cc5)CC4)CC3)nc3c2CCN(c2cc(O)cc4ccccc24)C3)CC1 ; "CF3COOH"]

### 1-(4-(2-((3-(4-((4-(4-(3-(2,4-dihydroxy-5-isopropylphenyl)-5-hydroxy-4H-1,2,4-triazol-4-yl)benzyl)piperidin-1-yl)methyl)piperidin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, CD3OD): δ 8.06 (d, J=8.4 Hz, 1H), 7.64 (d, J=8.0 Hz, 2H), 7.39-7.21 (m, 7H), 6.91-6.70 (m, 4H), 6.30-6.25 (m, 2H), 5.81 (d, J=10.4 Hz, 1H), 4.20-4.00 (m, 9H), 3.85-3.75 (m, 6H), 3.61-3.57 (m, 2H), 3.05-2.93 (m, 5H), 2.77-2.64 (m, 6H), 2.12-1.97 (m, 1H), 1.92-1.85 (m, 4H), 1.59-1.44 (m, 2H), 1.33 (s, 6H), 0.90 (d, J=6.4 Hz, 6H). LCMS (ESI): m/z found 990.7[M−CF3COOH+H]⁺.

### Compound 13

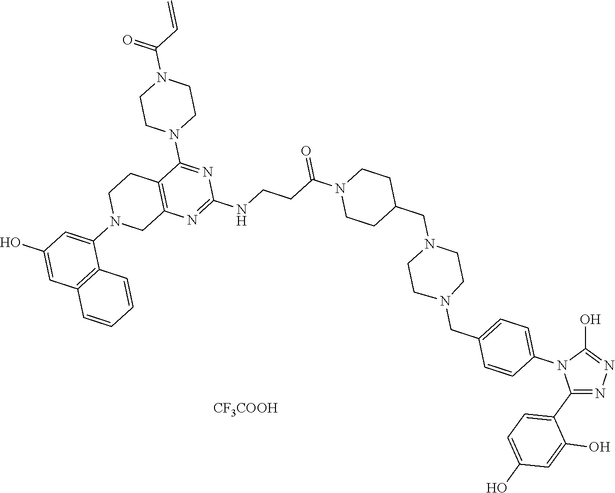

### 1-(4-(2-((3-(4-((4-(4-(3-(2,4-dihydroxyphenyl)-5-hydroxy-4H-1,2,4-triazol-4-yl)benzyl)piperazin-1-yl)methyl)piperidin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 8.05 (d, J=8.4 Hz, 1H), 7.64 (d, J=8.0 Hz, 1H), 7.48-7.25 (m, 6H), 7.03 (d, J=8.4 Hz, 1H), 6.93-6.75 (m, 3H), 6.31-6.15 (m, 3H), 5.84-5.78 (m, 1H), 4.59-4.49 (m, 2H), 4.18 (s, 2H), 4.04-3.94 (m, 7H), 3.88-3.72 (m, 6H), 3.19-2.93 (m, 11H), 2.82-2.73 (m, 4H), 2.71-2.62 (m, 1H), 2.08-1.98 (m, 1H), 1.90-1.78 (m, 2H), 1.38-1.06 (m, 3H). LCMS (ESI): m/z found 949.6[M−CF3COOH+H]⁺.

### Compound 14

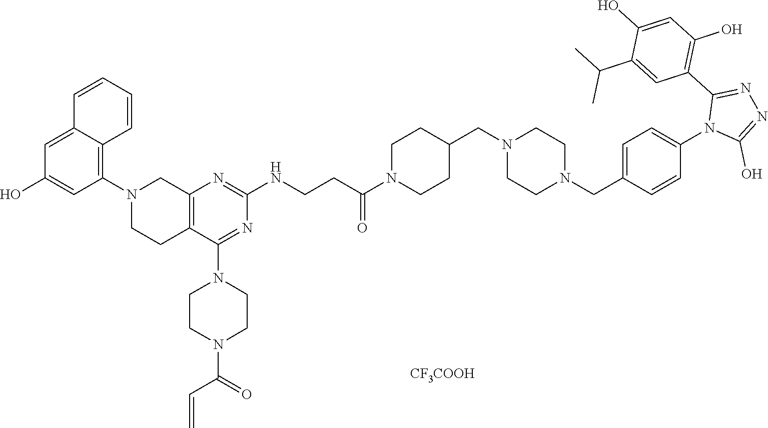

### 1-(4-(2-((3-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 8.06 (d, J=7.6 Hz, 1H), 7.72-7.28 (m, 8H), 6.95-6.77 (m, 4H), 6.30-6.22 (m, 2H), 5.83-5.76 (m, 1H), 4.48-4.38 (m, 4H), 4.27-3.67 (m, 14H), 3.40-3.29 (m, 2H), 3.17-2.65 (m, 15H), 2.10-1.73 (m, 3H), 1.37-1.10 (m, 4H), 1.00-0.87 (m, 7H). LCMS (ESI): found 991.1[M−CF3COOH+H]⁺.

### Compound 15

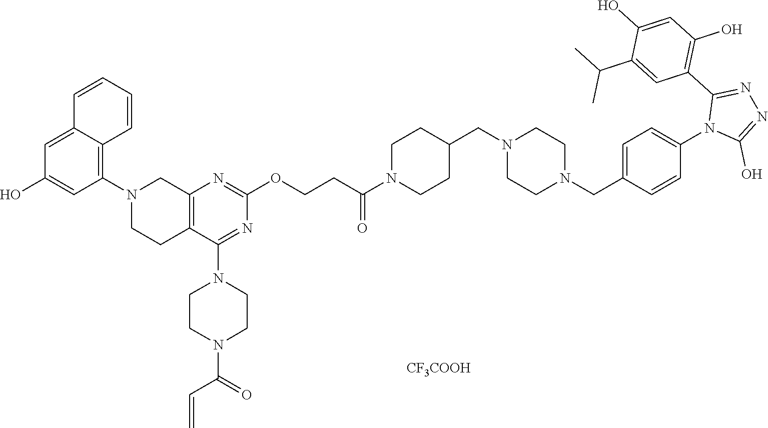

### 1-(4-(2-(3-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-3-oxopropoxy)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 11.97 (s, 1H), 9.80-9.30 (m, 2H), 8.00 (d, J=8.4 Hz, 1H), 7.74-7.18 (m, 7H), 6.92-6.75 (m, 3H), 6.33-6.12 (m, 2H), 5.78-5.70 (m, 1H), 4.54-4.33 (m, 3H), 4.16-3.83 (m, 12H), 3.58-3.42 (m, 7H), 3.34-2.73 (m, 16H), 2.08-1.71 (m, 3H), 1.26-1.10 (m, 2H), 1.00 (d, J=6.8 Hz, 6H). LCMS (ESI): found 992.1[M−CF3COOH+H]⁺.

### Compound 16

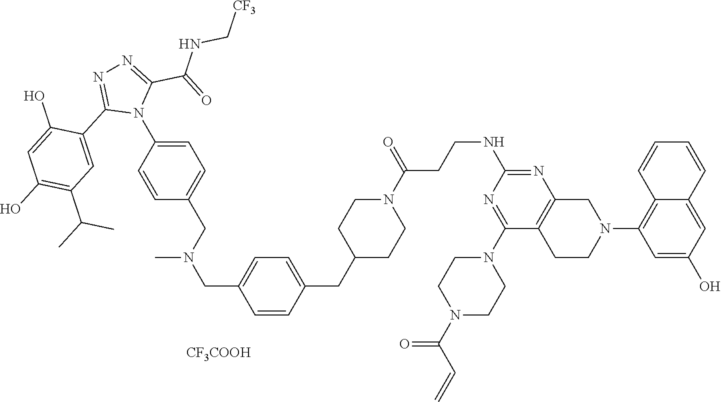 [SMILES: C=CC(=O)N1CCN(c2nc(NCCC(=O)N3CCC(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cc(O)cc4ccccc24)C3)CC1 ; "CF3COOH"]

### 4-(4-(((4-((1-(3-((4-(4-acryloylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)benzyl)(methyl)amino)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, CD₃OD) δ 8.06 (d, J=7.8 Hz, 1H), 7.66-7.25 (m, 12H), 6.93-6.75 (m, 4H), 6.32-6.23 (m, 2H), 5.81 (dd, J=10.6, 1.8 Hz, 1H), 4.19 (s, 1H), 4.02-3.70 (m, 12H), 3.06-2.93 (m, 3H), 2.74 (s, 5H), 2.68-2.54 (m, 2H), 2.08-1.81 (m, 1H), 1.77-1.56 (m, 4H), 1.32-1.25 (m, 10H), 1.24-1.05 (m, 4H), 0.97-0.93 (m, 7H). LCMS (ESI): m/z found 1135.3[M—CF3COOH+H]⁺.

### Compound 17

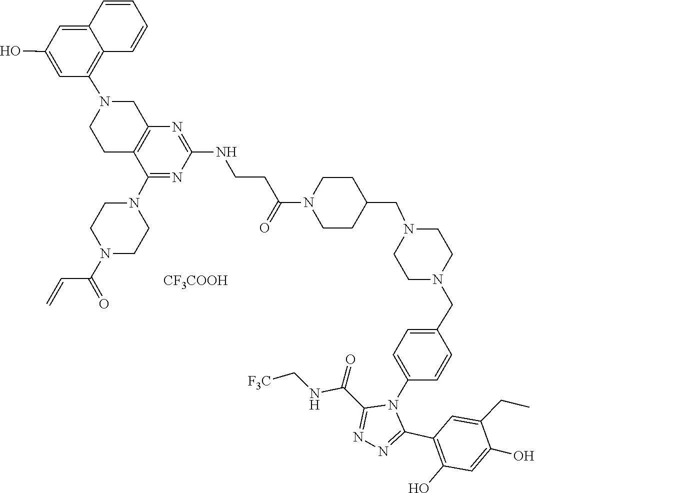

### 4-(4-((4-((1-(3-((4-(4-acryloylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(5-ethyl-2,4-dihydroxyphenyl)-N-(2,2,2-trifluoroethyl)-4H-1,2,4-triazole-3-carboxamide, trifluoroacetic acid

¹H NMR (400 MHz, DMSO-d₆): δ 9.91-9.50 (m, 3H), 7.99 (d, J=7.6 Hz, 1H), 7.69 (d, J=8.0 Hz, 1H), 7.48-7.14 (m, 7H), 6.94-6.60 (m, 5H), 6.35-6.11 (m, 2H), 5.75 (d, J=10.4 Hz, 1H), 5.36-5.28 (m, 1H), 4.39 (d, J=14.0 Hz, 1H), 4.16 (s, 1H), 4.02-3.90 (m, 4H), 3.90-3.79 (m, 7H), 3.01-2.90 (m, 7H), 2.69-2.26 (m, 4H), 2.30-2.24 (m, 5H), 2.03-1.97 (m, 4H), 1.74 (d, J=10.4 Hz, 3H), 1.50-1.37 (m, 3H), 0.87 (dd, J=15.6, 7.2 Hz, 6H). LCMS (ESI): m/z found 1086.7[M−CF3COOH+H]⁺.

### Compound 18

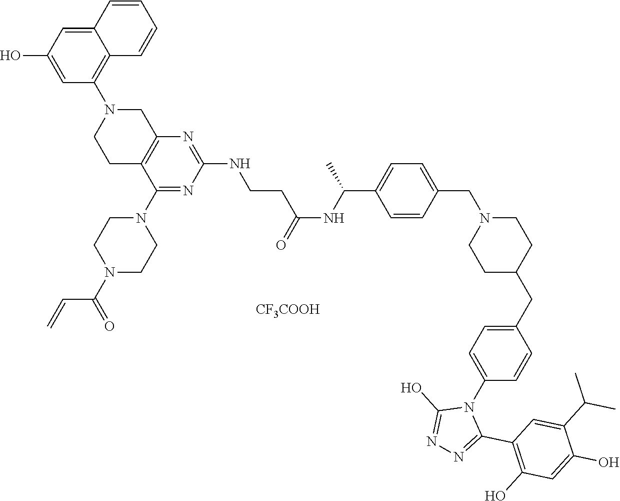 [SMILES: C=CC(=O)N1CCN(c2nc(NCCC(=O)N[C@H](C)c3ccc(CN4CCC(Cc5ccc(-n6c(O)nnc6-c6cc(C(C)C)c(O)cc6O)cc5)CC4)cc3)nc3c2CCN(c2cc(O)cc4ccccc24)C3)CC1 ; "CF3COOH"]

### (R)-3-((4-(4-acryloylpiperazin-1-yl)-7-(3-hydroxynaphthalen-1-yl)-5,6,7,8-tetrahydropyrido[3,4-d]pyrimidin-2-yl)amino)-N-(1-(4-((4-(4-(3-(2,4-dihydroxy-5-isopropylphenyl)-5-hydroxy-4H-1,2,4-triazol-4-yl)benzyl)piperidin-1-yl)methyl)phenyl)ethyl)propanamide, trifluoroacetic acid

¹H NMR (400 MHz, DMSO-d₆): δ 11.92 (s, 1H), 9.77-9.61 (m, 1H), 9.39-9.31 (m, 1H), 8.44 (s, 1H), 7.99 (d, J=8.4 Hz, 1H), 7.68 (d, J=8.6 Hz, 1H), 7.41-7.08 (m, 11H), 6.89-6.74 (m, 3H), 6.29-6.11 (m, 2H), 5.74 (d, J=12.6 Hz, 1H), 5.00-4.90 (m, 1H), 4.16 (d, J=31.2 Hz, 3H), 3.88-3.68 (m, 7H), 3.34-3.22 (m, 6H), 2.89-2.77 (m, 5H), 2.05-1.94 (m, 2H), 1.77-1.69 (m, 2H), 1.42-1.19 (m, 12H), 0.94 (d, J=6.8 Hz, 6H). LCMS (ESI): m/z found 1026.7[M−CF3COOH+H]⁺.

### Compound 19

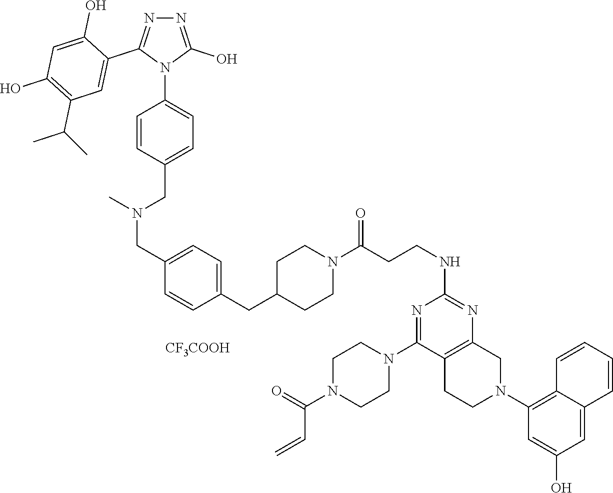 [SMILES: C=CC(=O)N1CCN(c2nc(NCCC(=O)N3CCC(Cc4ccc(CN(C)Cc5ccc(-n6c(O)nnc6-c6cc(C(C)C)c(O)cc6O)cc5)cc4)CC3)nc3c2CCN(c2cc(O)cc4ccccc24)C3)CC1 ; "CF3COOH"]

### 1-(4-(2-((3-(4-(4-(((4-(3-(2,4-dihydroxy-5-isopropylphenyl)-5-hydroxy-4H-1,2,4-triazol-4-yl)benzyl)(methyl)amino)methyl)benzyl)piperidin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, CD₃OD) δ 8.06 (d, J=8.1 Hz, 1H), 7.64 (d, J=8.2 Hz, 1H), 7.53 (d, J=8.2 Hz, 2H), 7.42-7.25 (m, 9H), 6.92-6.74 (m, 5H), 6.35-6.17 (m, 3H), 5.81 (d, J=12.1 Hz, 2H), 4.51 (m, 2H), 4.19 (s, 4H), 3.99 (s, 5H), 3.85 (s, 5H), 3.74 (s, 2H), 3.09-2.97 (m, 4H), 2.76-2.55 (m, 9H), 2.22-2.14 (m, 1H), 2.03 (d, J=5.5 Hz, 1H), 1.87 (s, 1H), 1.67 (m, 2H), 1.19-1.09 (m, 2H), 1.01 (d, J=6.9 Hz, 6H). LCMS (ESI): 1026.5[M−CF3COOH+H]⁺.

### Compound 20

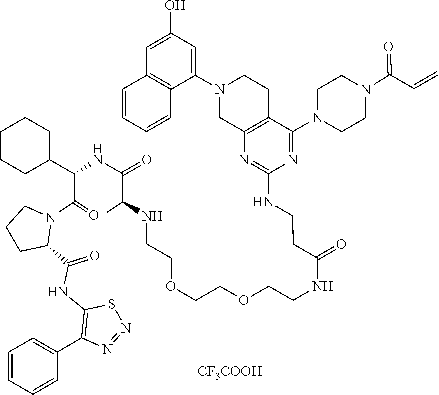 [SMILES: C=CC(=O)N1CCN(c2nc(NCCC(=O)NCCOCCOCCN[C@@H](C)C(=O)N[C@H](C(=O)N3CCC[C@H]3C(=O)Nc3snnc3-c3ccccc3)C3CCCCC3)nc3c2CCN(c2cc(O)cc4ccccc24)C3)CC1 ; "CF3COOH"]

### (S)-1-((2S,5S)-18-((4-(4-acryloylpiperazin-1-yl)-7-(3-hydroxynaphthalen-1-yl)-5,6,7,8-tetrahydropyrido[3,4-d]pyrimidin-2-yl)amino)-2-cyclohexyl-5-methyl-4,16-dioxo-9,12-dioxa-3,6,15-triazaoctadecanoyl)-N-(4-phenyl-1,2,3-thiadiazol-5-yl)pyrrolidine-2-carboxamide, trifluoroacetic acid

¹H NMR (400 MHz, CD₃OD) δ 8.06 (t, J=9.2 Hz, 1H), 7.81-7.46 (m, 7H), 7.39 (t, J=7.0 Hz, 1H), 7.29 (t, J=7.1 Hz, 1H), 6.90 (s, 1H), 6.86-6.74 (m, 2H), 6.28 (dd, J=16.8, 1.8 Hz, 1H), 5.81 (dd, J=10.6, 1.7 Hz, 1H), 4.83-4.79 (m, 1H), 4.50 (d, J=8.0 Hz, 1H), 4.27-3.35 (m, 28H), 3.22-2.90 (m, 5H), 2.56 (m, 2H), 2.10 (m, 4H), 1.86-1.63 (m, 6H), 1.53-1.43 (m, 3H), 1.37-0.98 (m, 8H). LCMS (ESI): m/z found 1100.0 [M−CF3COOH+H]⁺.

### Compound 21

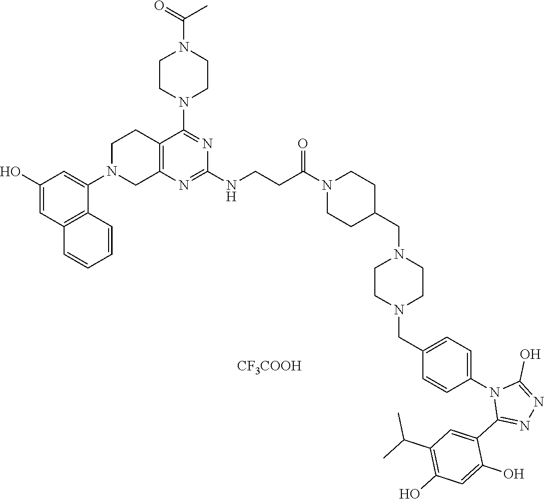

### 3-((4-(4-acetylpiperazin-1-yl)-7-(3-hydroxynaphthalen-1-yl)-5,6,7,8-tetrahydropyrido[3,4-d]pyrimidin-2-yl)amino)-1-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)propan-1-one, trifluoroacetic acid

¹H NMR (400 MHz, CD₃OD) δ 8.05 (d, J=8.5 Hz, 1H), 7.64 (d, J=8.2 Hz, 1H), 7.50 (d, J=8.3 Hz, 2H), 7.39 (t, J=7.4 Hz, 1H), 7.29 (m, 3H), 6.91 (d, J=1.8 Hz, 1H), 6.83-6.77 (m, 2H), 6.25 (s, 1H), 4.55 (d, J=12.6 Hz, 1H), 4.19 (s, 2H), 3.97 (m, 8H), 3.74 (s, 7H), 3.07 (m, 12H), 2.74 (m, 7H), 2.16 (s, 3H), 2.03 (s, 2H), 1.85 (t, J=13.5 Hz, 2H), 1.31-1.08 (m, 4H), 0.92 (m, 7H). LCMS (ESI): m/z found 979.1 [M−CF3COOH+H]⁺.

### Compound 22

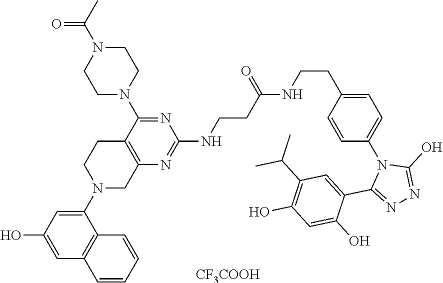 [SMILES: CC(=O)N1CCN(c2nc(NCCC(=O)NCCc3ccc(-n4c(O)nnc4-c4cc(C(C)C)c(O)cc4O)cc3)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)phenethyl)propanamide, trifluoroacetic acid

¹H NMR (400 MHz, CD₃OD): δ 8.05 (d, J=8.4 Hz, 1H), 7.64 (d, J=8.0 Hz, 1H), 7.40-7.17 (m, 6H), 6.91-6.68 (m, 3H), 6.26 (s, 1H), 5.38-5.34 (m, 1H), 4.19 (s, 2H), 4.01-3.91 (m, 3H), 3.75-3.68 (m, 6H), 3.45 (t, J=7.2 Hz, 2H), 3.01-2.81 (m, 5H), 2.53 (t, J=6.4 Hz, 2H), 2.19-1.98 (m, 4H), 1.68-1.53 (m, 1H), 0.88 (d, J=6.8 Hz, 6H). LCMS (ESI): m/z found 827.1[M−CF3COOH+H]⁺.

### Compound 23

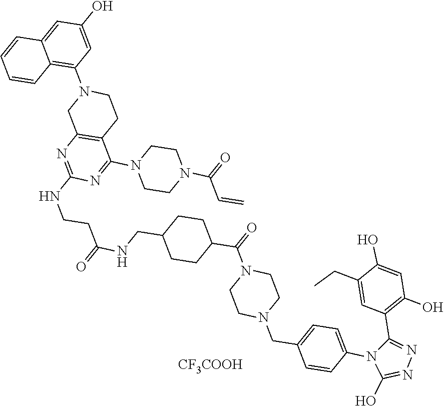 [SMILES: C=CC(=O)N1CCN(c2nc(NCCC(=O)NCC3CCC(C(=O)N4CCN(Cc5ccc(-n6c(O)nnc6-c6cc(CC)c(O)cc6O)cc5)CC4)CC3)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-((4-(4-(4-(3-(5-ethyl-2,4-dihydroxyphenyl)-5-hydroxy-4H-1,2,4-triazol-4-yl)benzyl)piperazine-1-carbonyl)cyclohexyl)methyl)propanamide, trifluoroacetic acid

¹H NMR (400 MHz, DMSO-d₆): δ 12.45 (s, 1H), 10.46 (s, 1H), 9.79-9.62 (m, 3H), 8.11-7.99 (m, 2H), 7.69 (d, J=6.8 Hz, 1H), 7.41-7.27 (m, 10H), 6.89 (s, 1H), 6.75 (s, 1H), 6.60 (s, 1H), 6.34 (s, 1H), 6.19-6.15 (m, 1H), 5.76-5.72 (m, 1H), 4.24-4.15 (m, 4H), 4.03-3.90 (m, 3H), 3.80-3.60 (m, 6H), 3.43-3.22 (m, 8H), 2.80-2.61 (m, 6H), 2.44-2.38 (m, 3H), 1.78-1.71 (m, 3H), 1.45-1.38 (m, 2H), 1.24 (s, 2H), 0.82 (d, J=6.4 Hz, 6H). LCMS (ESI): m/z found 1020.0[M−CF3COOH+H]⁺.

### Compound 24

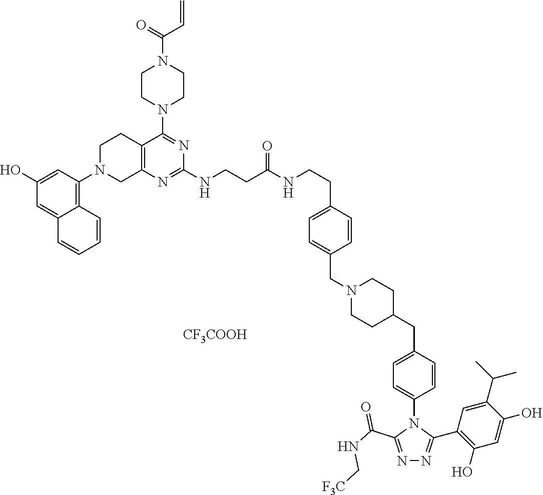 [SMILES: C=CC(=O)N1CCN(c2nc(NCCC(=O)NCCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; "CF3COOH"]

### 4-(4-((1-(4-(2-(3-((4-(4-acryloylpiperazin-1-yl)-7-(3-hydroxynaphthalen-1-yl)-5,6,7,8-tetrahydropyrido[3,4-d]pyrimidin-2-yl)amino)propanamido)ethyl)benzyl)piperidin-4-yl)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆): δ 10.48 (s, 1H), 9.89-9.60 (m, 3H), 8.15-7.93 (m, 3H), 7.71-7.68 (m, 2H), 7.45-7.22 (m, 11H), 6.92-6.52 (m, 4H), 6.37-6.21 (m, 1H), 5.71-5.68 (m, 1H), 5.38 (s, 1H), 4.23-4.12 (m, 4H), 3.97-3.97 (m, 2H), 3.75-3.66 m, 6H), 2.96-2.90 (m, 5H), 2.76-2.67 (m, 5H), 2.35-2.33 (m, 5H), 2.04-1.95 (m, 4H), 1.74-1.72 (m, 2H), 1.48-1.37 (m, 2H), 0.85-0.80 (m, 8H). LCMS (ESI): m/z found 1135.5[M−CF3COOH+H]⁺.

### Compound 25

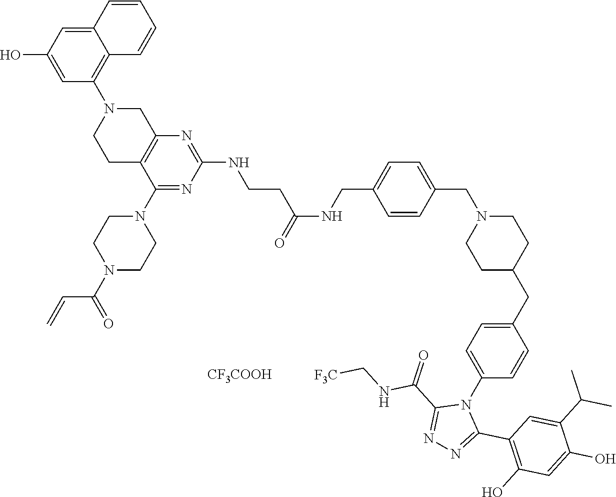 [SMILES: C=CC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; "CF3COOH"]

### 4-(4-((1-(4-((3-((4-(4-acryloylpiperazin-1-yl)-7-(3-hydroxynaphthalen-1-yl)-5,6,7,8-tetrahydropyrido[3,4-d]pyrimidin-2-yl)amino)propanamido)methyl)benzyl)piperidin-4-yl)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆): δ 9.78 (s, 1H), 9.61 (s, 1H), 9.58 (s, 1H), 8.54 (s, 1H), 7.99 (d, J=8.4 Hz, 1H), 7.68 (d, J=8.4 Hz, 1H), 7.43-7.40 (m, 4H), 7.35-7.26 (m, 8H), 6.96-6.75 (m, 3H), 6.60 (s, 1H), 6.34 (s, 1H), 6.18 (d, J=10.4 Hz, 1H), 5.75 (d, J=8.4 Hz, 1H), 4.31 (s, 3H), 4.24 (s, 2H), 4.15 (s, 2H), 3.88-3.76 (m, 7H), 3.34-3.21 (m, 7H), 2.97-2.78 (m, 8H), 1.78-1.70 (m, 3H), 1.39-1.25 (m, 2H), 1.23 (s, 2H), 0.82 (d, J=6.0 Hz, 6H). LCMS (ESI): m/z found 1121.5[M−CF3COOH+H]⁺.

### Compound 26

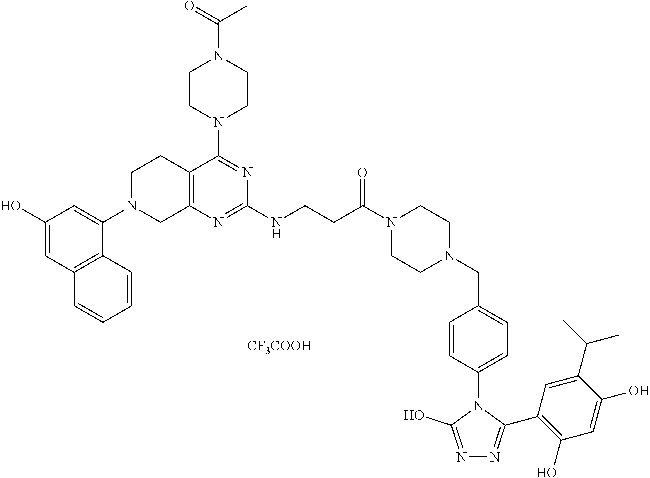 [SMILES: CC(=O)N1CCN(c2nc(NCCC(=O)N3CCN(Cc4ccc(-n5c(O)nnc5-c5cc(C(C)C)c(O)cc5O)cc4)CC3)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-1-(4-(4-(3-(2,4-dihydroxy-5-isopropylphenyl)-5-hydroxy-4H-1,2,4-triazol-4-yl)benzyl)piperazin-1-yl)propan-1-one, trifluoroacetic acid

¹H NMR (400 MHz, CD₃OD): δ 8.06 (d, J=6.0 Hz, 1H), 7.75-7.60 (m, 3H), 7.41-7.17 (m, 6H), 6.85-6.66 (m, 3H), 6.27 (s, 1H), 4.57 (s, 1H), 4.12-4.06 (m, 3H), 3.72-3.48 (m, 13H), 3.02-2.69 (m, 5H), 2.41-2.36 (m, 4H), 2.15 (s, 3H), 2.06-1.98 (m, 1H), 0.87 (d, J=7.6 Hz, 6H). LCMS (ESI): m/z found 882.0[M−CF3COOH+H]⁺.

### Compound 27

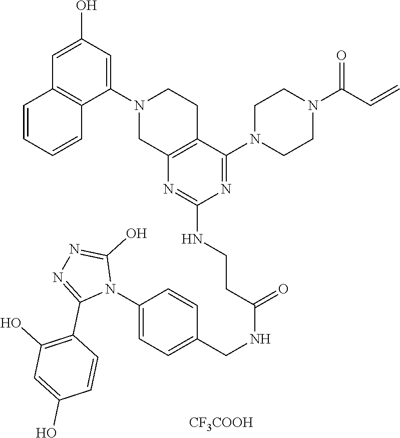 [SMILES: C=CC(=O)N1CCN(c2nc(NCCC(=O)NCc3ccc(-n4c(O)nnc4-c4ccc(O)cc4O)cc3)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxyphenyl)-5-hydroxy-4H-1,2,4-triazol-4-yl)benzyl)propanamide, trifluoroacetic acid

¹H NMR (400 MHz, CD₃OD) δ 8.04 (d, J=8.7 Hz, 1H), 7.64 (d, J=8.2 Hz, 1H), 7.39 (t, J=7.1 Hz, 1H), 7.28 (dd, J=13.1, 7.9 Hz, 3H), 7.16 (d, J=8.1 Hz, 2H), 6.92 (d, J=8.2 Hz, 2H), 6.77 (dd, J=16.9, 10.7 Hz, 2H), 6.31-6.15 (m, 3H), 5.80 (dd, J=10.6, 1.8 Hz, 1H), 4.39 (s, 2H), 4.17 (s, 2H), 3.97 (s, 4H), 3.80 (s, 6H), 2.96 (s, 2H), 2.60 (t, J=6.1 Hz, 2H). LCMS (ESI): m/z found 783.3 [M−CF3COOH+H]⁺.

### Compound 28

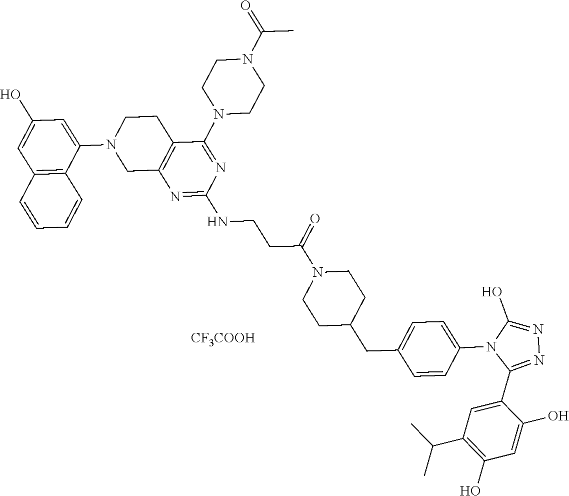 [SMILES: CC(=O)N1CCN(c2nc(NCCC(=O)N3CCC(Cc4ccc(-n5c(O)nnc5-c5cc(C(C)C)c(O)cc5O)cc4)CC3)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-1-(4-(4-(3-(2,4-dihydroxy-5-isopropylphenyl)-5-hydroxy-4H-1,2,4-triazol-4-yl)benzyl)piperidin-1-yl)propan-1-one, trifluoroacetic acid

¹H NMR (400 MHz, CD₃OD) δ 8.06 (d, J=8.7 Hz, 1H), 7.64 (d, J=8.4 Hz, 1H), 7.48-7.13 (m, 6H), 6.91 (s, 1H), 6.81 (s, 1H), 6.67 (s, 1H), 6.28 (s, 1H), 5.34 (s, 1H), 4.51 (s, 1H), 4.19 (s, 2H), 3.95 (d, J=19.4 Hz, 4H), 3.61 (m, 12H), 3.00 (m, 5H), 2.75 (s, 2H), 2.67-2.53 (m, 3H), 2.17 (m, 3H), 2.04 (s, 1H), 1.85 (s, 1H), 1.73 (s, 2H), 1.60 (s, 1H), 0.88 (t, J=7.6 Hz, 6H). LCMS (ESI): m/z found 881.8 [M−CF3COOH+H]⁺.

### Compound 29

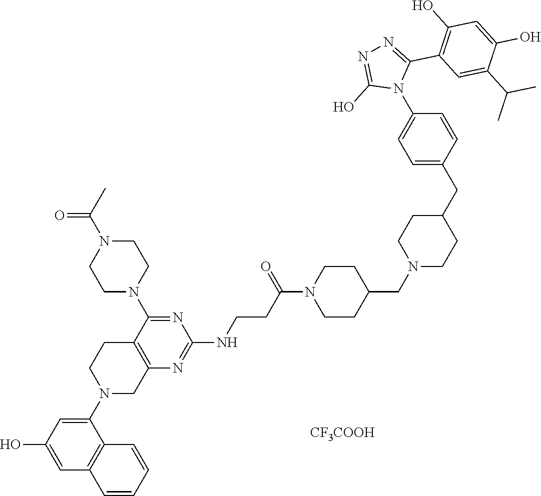 [SMILES: CC(=O)N1CCN(c2nc(NCCC(=O)N3CCC(CN4CCC(Cc5ccc(-n6c(O)nnc6-c6cc(C(C)C)c(O)cc6O)cc5)CC4)CC3)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-1-(4-((4-(4-(3-(2,4-dihydroxy-5-isopropylphenyl)-5-hydroxy-4H-1,2,4-triazol-4-yl)benzyl)piperidin-1-yl)methyl)piperidin-1-yl)propan-1-one, trifluoroacetic acid

¹H NMR (400 MHz, CD₃OD): δ 8.06 (d, J=8.0 Hz, 1H), 7.64 (d, J=8.4 Hz, 1H), 7.46-7.16 (m, 8H), 6.97-6.64 (m, 3H), 6.27 (s, 1H), 5.34 (t, J=4.8 Hz, 2H), 4.64-4.53 (m, 1H), 4.18 (s, 2H), 4.04-3.90 (m, 5H), 3.74 (s, 6H), 3.60-3.53 (m, 10H), 2.98-2.91 (m, 5H), 2.82-2.74 (m, 4H), 2.05-2.00 (m, 3H), 1.95-1.78 (m, 6H), 1.64-1.55 (m, 3H), 0.91-0.89 (m, 6H). LCMS (ESI): m/z found 979.0[M−CF3COOH+H]⁺.

### Compound 30

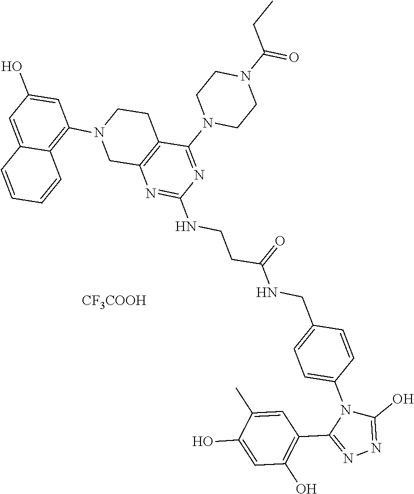 [SMILES: CCC(=O)N1CCN(c2nc(NCCC(=O)NCc3ccc(-n4c(O)nnc4-c4cc(C)c(O)cc4O)cc3)nc3c2CCN(c2cc(O)cc4ccccc24)C3)CC1 ; "CF3COOH"]

### N-(4-(3-(2,4-dihydroxy-5-methylphenyl)-5-hydroxy-4H-1,2,4-triazol-4-yl)benzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆): δ 11.86 (s, 1H), 9.74 (s, 1H), 9.55 (s, 1H), 9.30 (s, 1H), 8.49 (s, 1H), 7.99 (d, J=8.0 Hz, 1H), 7.69 (d, J=8.4 Hz, 1H), 7.49-6.67 (m, 10H), 6.22 (s, 1H), 4.27 (d, J=5.8 Hz, 2H), 4.16 (s, 2H), 3.86 (s, 4H), 3.61 (s, 6H), 3.24 (s, 2H), 2.91 (s, 2H), 2.72-2.64 (m, 1H), 2.41-2.31 (m, 3H), 1.95 (s, 3H), 1.01 (t, J=7.6 Hz, 3H). LCMS (ESI): m/z found 799.3[M−CF3COOH+H]⁺.

### Compound 31

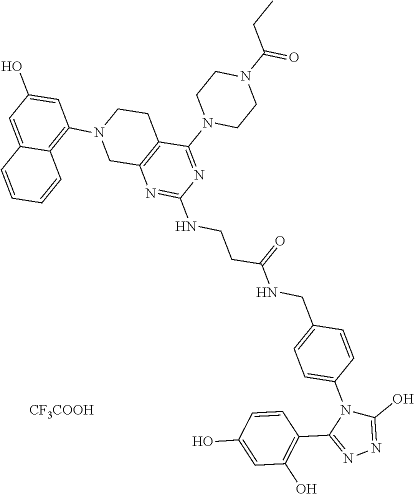 [SMILES: "CF3COOH" ; CCC(=O)N1CCN(c2nc(NCCC(=O)NCc3ccc(-n4c(O)nnc4-c4ccc(O)cc4O)cc3)nc3c2CCN(c2cc(O)cc4ccccc24)C3)CC1]

### N-(4-(3-(2,4-dihydroxyphenyl)-5-hydroxy-4H-1,2,4-triazol-4-yl)benzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆): δ 11.88 (s, 1H), 9.84-9.52 (m, 3H), 8.55-8.45 (m, 1H), 7.99 (d, J=8.4 Hz, 1H), 7.69 (d, J=8.0 Hz, 2H), 7.50-6.65 (m, 10H), 6.25-6.11 (m, 2H), 4.33-4.07 (m, 5H), 3.94-3.78 (m, 6H), 3.55-3.54 (m, 2H), 3.30-3.18 (m, 3H), 2.98-2.86 (m, 2H), 2.40-2.31 (m, 4H), 1.04-0.98 (m, 3H). LCMS (ESI): m/z found 785.3[M−CF3COOH+H]⁺.

### Compound 32

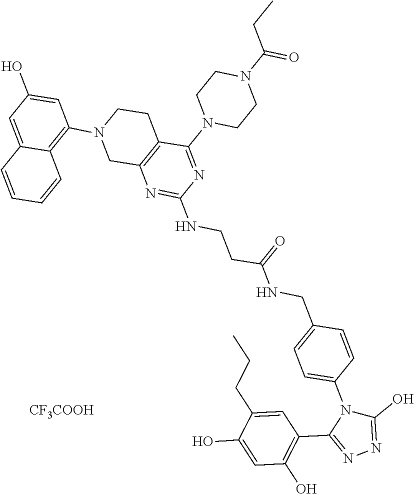 [SMILES: "CF3COOH" ; CCCc1cc(-c2nnc(O)n2-c2ccc(CNC(=O)CCNc3nc4c(c(N5CCN(C(=O)CC)CC5)n3)CCN(c3cc(O)cc5ccccc35)C4)cc2)c(O)cc1O]

### N-(4-(3-(2,4-dihydroxy-5-propylphenyl)-5-hydroxy-4H-1,2,4-triazol-4-yl)benzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆): δ 11.87 (s, 1H), 9.74 (s, 1H), 9.53-9.29 (m, 2H), 8.48 (s, 1H), 7.98 (d, J=8.4 Hz, 1H), 7.68 (d, J=8.0 Hz, 1H), 7.45-6.70 (m, 10H), 6.23 (s, 1H), 4.30-4.13 (m, 4H), 3.95-3.75 (m, 4H), 3.64-3.58 (m, 6H), 3.27-3.20 (m, 2H), 2.97-2.85 (m, 2H), 2.41-2.27 (m, 5H), 1.45-1.35 (m, 2H), 1.01 (t, J=7.2 Hz, 3H), 0.78 (t, J=7.2 Hz, 3H). LCMS (ESI): m/z found 827.7[M−CF3COOH+H]⁺.

### Compound 33

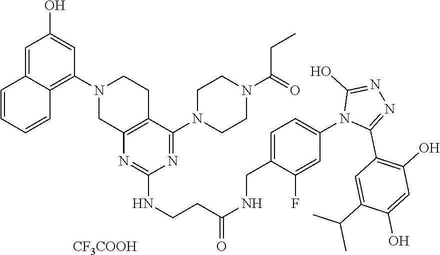 [SMILES: CCC(=O)N1CCN(c2nc(NCCC(=O)NCc3ccc(-n4c(O)nnc4-c4cc(C(C)C)c(O)cc4O)cc3F)nc3c2CCN(c2cc(O)cc4ccccc24)C3)CC1 ; "CF3COOH"]

### N-(4-(3-(2,4-dihydroxy-5-isopropylphenyl)-5-hydroxy-4H-1,2,4-triazol-4-yl)-2-fluorobenzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆): δ 11.95 (s, 1H), 9.75-9.57 (m, 2H), 9.37 (s, 1H), 8.47 (s, 1H), 7.99 (d, J=8.0 Hz, 1H), 7.68 (d, J=8.0 Hz, 1H), 7.45-7.22 (m, 3H), 7.13-6.69 (m, 6H), 6.24 (s, 1H), 5.98 (s, 1H), 5.52 s, 2H), 4.33-4.25 (m, 3H), 4.17-4.09 (s, 2H), 3.94-3.74 (m, 5H), 3.63-3.58 (m, 5H), 3.06-2.86 (m, 4H), 2.39-2.32 (m, 3H), 1.05-1.01 (m, 9H). LCMS (ESI): m/z found 845.7[M−CF3COOH+H]⁺.

### Compound 34

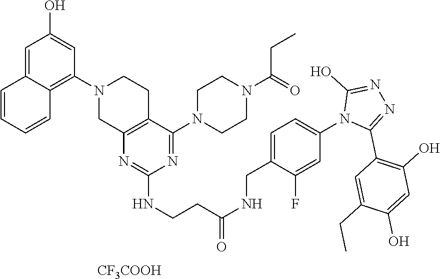 [SMILES: CCC(=O)N1CCN(c2nc(NCCC(=O)NCc3ccc(-n4c(O)nnc4-c4cc(CC)c(O)cc4O)cc3F)nc3c2CCN(c2cc(O)cc4ccccc24)C3)CC1 ; "CF3COOH"]

### N-(4-(3-(5-ethyl-2,4-dihydroxyphenyl)-5-hydroxy-4H-1,2,4-triazol-4-yl)-2-fluorobenzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆): δ 11.95 (s, 1H), 9.74 (s, 1H), 9.58 (s, 1H), 9.37 (s, 1H), 8.48 (s, 1H), 7.99 (d, J=8.4 Hz, 1H), 7.68 (d, J=8.8 Hz, 1H), 7.44-7.38 (m, 1H), 7.32-7.20 (m, 2H), 7.10-7.02 (m, 1H), 6.98-6.88 (m, 3H), 6.75 (s, 1H), 6.24 (s, 1H), 4.33-4.25 (m, 2H), 4.19-4.10 (m, 2H), 3.95-3.79 (m, 4H), 3.66-3.54 (m, 8H), 3.27-3.20 (m, 2H), 2.99-2.83 (m, 2H), 2.41-2.32 (m, 5H), 1.05-0.98 (m, 6H). LCMS (ESI): m/z found 831.3[M−CF3COOH+H]⁺.

### Compound 35

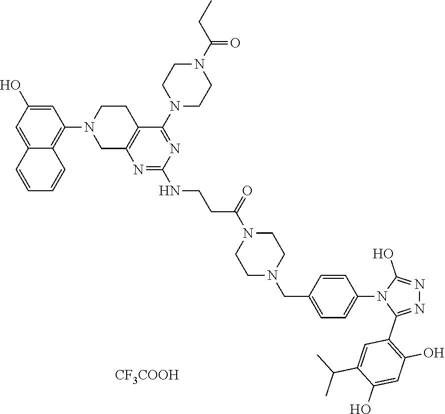 [SMILES: CCC(=O)N1CCN(c2nc(NCCC(=O)N3CCN(Cc4ccc(-n5c(O)nnc5-c5cc(C(C)C)c(O)cc5O)cc4)CC3)nc3c2CCN(c2cc(O)cc4ccccc24)C3)CC1 ; "CF3COOH"]

### 1-(4-(4-(3-(2,4-dihydroxy-5-isopropylphenyl)-5-hydroxy-4H-1,2,4-triazol-4-yl)benzyl)piperazin-1-yl)-3-((7-(3-hydroxynaphthalen-1-yl)-4-(4-propionylpiperazin-1-yl)-5,6,7,8-tetrahydropyrido[3,4-d]pyrimidin-2-yl)amino)propan-1-one, trifluoroacetic acid

¹H NMR (400 MHz, DMSO-d₆): δ 11.96 (s, 1H), 9.80-9.58 (m, 2H), 9.34 (s, 1H), 7.99 (d, J=8.8 Hz, 1H), 7.68 (d, J=8.0 Hz, 1H), 7.54-7.20 (m, 6H), 6.89-6.73 (m, 2H), 6.25 (s, 1H), 4.45-4.25 (m, 2H), 4.21-4.09 (m, 2H), 3.90-3.76 (m, 5H), 3.71-3.59 (m, 11H), 3.41-3.36 (m, 4H), 3.29-3.22 (m, 3H), 3.03-2.90 (m, 4H), 2.71-2.65 (m, 3H), 2.40-2.31 (m, 3H), 1.06-0.94 (m, 9H). LCMS (ESI): S m/z found 896.4[M−CF3COOH+H]⁺.

### Compound 36

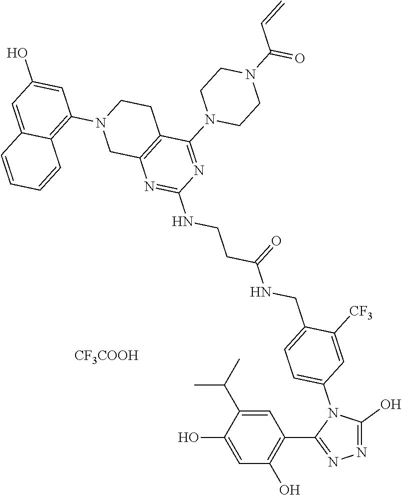 [SMILES: C=CC(=O)N1CCN(c2nc(NCCC(=O)NCc3ccc(-n4c(O)nnc4-c4cc(C(C)C)c(O)cc4O)cc3C(F)(F)F)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)-2-(trifluoromethyl)benzyl)propanamide, trifluoroacetic acid. ¹H NMR (400 MHz, DMSO-d₆): δ 12.01 (s, 1H), 9.66 (d, J=58.8 Hz, 2H), 9.35 (s, 1H), 8.58 (s, 1H), 7.99 (d, J=8.8 Hz, 1H), 7.69 (d, J=8.0 Hz, 1H), 7.55-7.25 (m, 5H), 6.99-6.72 (m, 4H), 6.26-6.12 (m, 2H), 5.77-5.70 (m, 1H), 4.43 (d, J=7.0 Hz, 2H), 4.15 (s, 2H), 3.88 (s, 3H), 3.78-3.68 (m, 4H), 3.63-3.58 (m, 3H), 3.31-3.23 (m, 3H), 3.05-3.01 (m, 1H), 2.97-2.87 (m, 2H), 2.60-2.55 (m, 2H), 1.03 (d, J=6.8 Hz, 6H). LCMS (ESI): m/z found 893.3[M−CF3COOH+H]⁺

### Compound 37

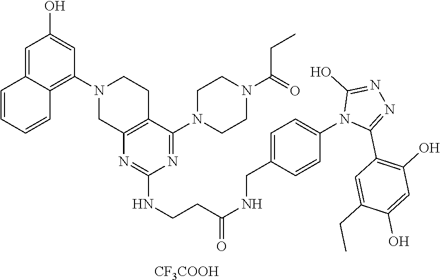 [SMILES: CCC(=O)N1CCN(c2nc(NCCC(=O)NCc3ccc(-n4c(O)nnc4-c4cc(CC)c(O)cc4O)cc3)nc3c2CCN(c2cc(O)cc4ccccc24)C3)CC1 ; "CF3COOH"]

### N-(4-(3-(5-ethyl-2,4-dihydroxyphenyl)-5-hydroxy-4H-1,2,4-triazol-4-yl)benzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆): δ 11.87 (s, 1H), 9.73 (s, 1H), 9.53 (s, 1H), 9.33 (s, 1H), 8.47 (s, 1H), 7.99 (d, J=7.8 Hz, 1H), 7.68 (d, J=8.2 Hz, 1H), 7.45-7.06 (m, 7H), 6.88-6.75 (m, 3H), 6.23 (s, 1H), 4.27 (d, J=5.8 Hz, 2H), 4.12 (s, 2H), 3.89-3.75 (m, 2H), 3.60 (s, 6H), 3.27-3.19 (m, 3H), 2.89 (s, 2H), 2.35 (dd, J=14.8, 7.2 Hz, 6H), 1.00 (t, J=7.2 Hz, 7H). LCMS (ESI): m/z found 813.2[M−CF3COOH+H]⁺.

### Compound 38

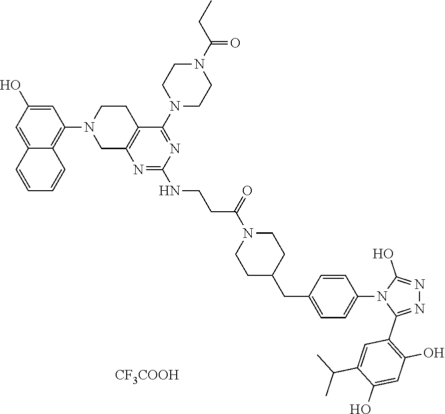 [SMILES: CCC(=O)N1CCN(c2nc(NCCC(=O)N3CCC(Cc4ccc(-n5c(O)nnc5-c5cc(C(C)C)c(O)cc5O)cc4)CC3)nc3c2CCN(c2cc(O)cc4ccccc24)C3)CC1 ; "CF3COOH"]

### 1-(4-(4-(3-(2,4-dihydroxy-5-isopropylphenyl)-5-hydroxy-4H-1,2,4-triazol-4-yl)benzyl)piperidin-1-yl)-3-((7-(3-hydroxynaphthalen-1-yl)-4-(4-propionylpiperazin-1-yl)-5,6,7,8-tetrahydropyrido[3,4-d]pyrimidin-2-yl)amino)propan-1-one, trifluoroacetic acid

¹H NMR (400 MHz, DMSO-d₆): δ 11.91 (s, 1H), 9.73 (s, 1H), 9.60 (s, 1H), 9.42 (s, 1H), 7.99 (d, J=8.2 Hz, 1H), 7.68 (d, J=8.7 Hz, 1H), 7.41-7.09 (m, 6H), 6.93-6.84 (m, 1H), 6.79-6.68 (m, 2H), 6.27 (s, 1H), 4.42-4.32 (m, 1H), 4.11 (s, 1H), 3.83-3.74 (m, 3H), 3.67-3.48 (m, 9H), 3.43-3.39 (m, 5H), 3.26-3.19 (m, 3H), 3.01-2.86 (m, 3H), 2.69-2.60 (m, 2H), 2.41-2.30 (m, 3H), 1.79-1.67 (m, 1H), 1.63-1.51 (m, 2H), 1.01 (t, J=7.4 Hz, 3H), 0.92 (d, J=6.8 Hz, 6H). LCMS (ESI): m/z found 895.4[M−CF3COOH+H]⁺.

### Compound 39

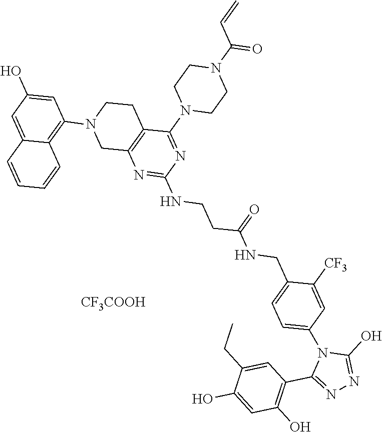 [SMILES: C=CC(=O)N1CCN(c2nc(NCCC(=O)NCc3ccc(-n4c(O)nnc4-c4cc(CC)c(O)cc4O)cc3C(F)(F)F)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(4-(3-(5-ethyl-2,4-dihydroxyphenyl)-5-hydroxy-4H-1,2,4-triazol-4-yl)-2-(trifluoromethyl)benzyl)propanamide, trifluoroacetic acid

¹H NMR (400 MHz, DMSO) δ 12.01 (s, 1H), 9.75 (s, 1H), 9.58 (s, 1H), 9.36 (s, 1H), 8.58 (s, 1H), 7.99 (d, J=8.4 Hz, 1H), 7.69 (d, J=8.4 Hz, 1H), 7.54 (s, 1H), 7.42 (m, 3H), 7.33-7.24 (m, 1H), 6.97 (d, J=5.9 Hz, 1H), 6.89 (s, 1H), 6.85-6.72 (m, 2H), 6.18 (m, 2H), 5.74 (d, J=12.6 Hz, 1H), 4.43 (d, J=5.4 Hz, 2H), 4.15 (s, 2H), 3.87 (s, 3H), 3.72 (m, 4H), 3.62 (s, 3H), 3.24 (s, 2H), 2.91 (s, 2H), 2.56 (d, J=6.7 Hz, 2H), 2.37 (m, 3H), 1.02 (t, J=7.4 Hz, 3H). LCMS (ESI): m/z found 879.0 [M−CF3COOH+H]⁺.

### Compound 40

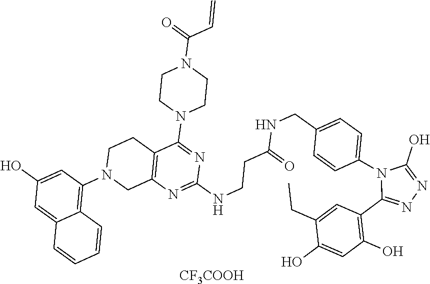 [SMILES: C=CC(=O)N1CCN(c2nc(NCCC(=O)NCc3ccc(-n4c(O)nnc4-c4cc(CC)c(O)cc4O)cc3)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(4-(3-(5-ethyl-2,4-dihydroxyphenyl)-5-hydroxy-4H-1,2,4-triazol-4-yl)benzyl)propanamide, trifluoroacetic acid

¹H NMR (400 MHz, DMSO-d₆) δ 11.88 (s, 1H), 9.77 (s, 1H), 9.56 (s, 1H), 9.34 (s, 1H), 8.50 (s, 1H), 7.99 (d, J=8.4 Hz, 1H), 7.81 (s, 1H), 7.69 (d, J=8.1 Hz, 1H), 7.41 (t, J=7.5 Hz, 1H), 7.33-7.25 (m, 1H), 7.21 (d, J=8.4 Hz, 2H), 7.11 (d, J=8.4 Hz, 2H), 6.88 (d, J=9.8 Hz, 2H), 6.84-6.72 (m, 2H), 6.26-6.09 (m, 2H), 5.78-5.70 (m, 1H), 4.27 (d, J=5.7 Hz, 2H), 4.16 (s, 2H), 3.88 (s, 4H), 3.72 (d, J=22.9 Hz, 4H), 3.62 (s, 3H), 3.24 (s, 2H), 2.92 (s, 2H), 2.53 (s, 1H), 2.35 (m, 2H), 0.99 (t, J=7.5 Hz, 3H). LCMS (ESI): m/z found 881.3 [M—CF3COOH+H]⁺.

### Compound 41

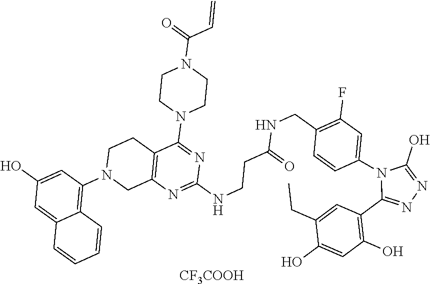 [SMILES: C=CC(=O)N1CCN(c2nc(NCCC(=O)NCc3ccc(-n4c(O)nnc4-c4cc(CC)c(O)cc4O)cc3F)nc3c2CCN(c2cc(O)cc4ccccc24)C3)CC1 ; "CF3COOH"]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(4-(3-(5-ethyl-2,4-dihydroxyphenyl)-5-hydroxy-4H-1,2,4-triazol-4-yl)-2-fluorobenzyl)propanamide, trifluoroacetic acid

¹H NMR (400 MHz, DMSO-d₆) δ 11.95 (s, 1H), 9.76 (s, 1H), 9.59 (s, 1H), 9.38 (s, 1H), 8.49 (s, 1H), 7.99 (d, J=8.6 Hz, 1H), 7.82 (s, 1H), 7.69 (d, J=8.2 Hz, 1H), 7.41 (t, J=7.4 Hz, 1H), 7.33-7.20 (m, 2H), 7.05 (d, J=11.0 Hz, 1H), 6.92 (m, 3H), 6.87-6.71 (m, 2H), 6.24 (s, 1H), 6.16 (m, 1H), 5.74 (d, J=12.6 Hz, 1H), 4.29 (d, J=5.3 Hz, 2H), 4.15 (s, 2H), 3.88 (s, 4H), 3.72 (m, 5H), 3.59 (s, 2H), 3.25 (s, 2H), 2.92 (s, 2H), 2.38 (dd, J=14.9, 7.5 Hz, 2H), 1.02 (t, J=7.5 Hz, 3H). LCMS (ESI): m/z found 829.3 [M−CF3COOH+H]⁺.

### Compound 42

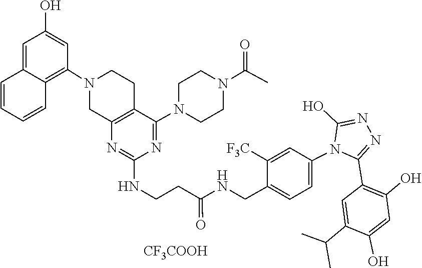 [SMILES: CC(=O)N1CCN(c2nc(NCCC(=O)NCc3ccc(-n4c(O)nnc4-c4cc(C(C)C)c(O)cc4O)cc3C(F)(F)F)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)-2-(trifluoromethyl)benzyl)propanamide, trifluoroacetic acid

¹H NMR (400 MHz, DMSO-d₆): δ 12.01 (s, 1H), 9.75-9.35 (m, 3H), 8.58 (s, 1H), 7.80-7.67 (m, 3H), 7.51-7.26 (m, 5H), 6.97-6.75 (m, 3H), 6.23 (s, 1H), 4.43 (d, J=4.4 Hz, 2H), 4.15 (s, 2H), 3.86-3.77 (m, 4H), 3.60 (s, 6H), 3.29 (s, 2H), 3.04-2.88 (m, 3H), 2.58-2.54 (m, 2H), 2.04 (s, 3H), 1.03 (d, J=6.8 Hz, 7H). LCMS (ESI): m/z found 881.0[M−CF3COOH+H]⁺.

### Compound 43

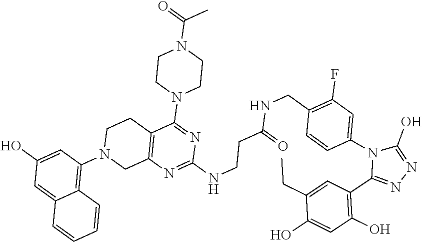 [SMILES: CCc1cc(-c2nnc(O)n2-c2ccc(CNC(=O)CCNc3nc4c(c(N5CCN(C(C)=O)CC5)n3)CCN(c3cc(O)cc5ccccc35)C4)c(F)c2)c(O)cc1O]

### 3-((4-(4-acetylpiperazin-1-yl)-7-(3-hydroxynaphthalen-1-yl)-5,6,7,8-tetrahydropyrido[3,4-d]pyrimidin-2-yl)amino)-N-(4-(3-(5-ethyl-2,4-dihydroxyphenyl)-5-hydroxy-4H-1,2,4-triazol-4-yl)-2-fluorobenzyl)propanamide

¹H NMR (400 MHz, DMSO-d₆): δ 11.95 (s, 1H), 9.76-9.37 (m, 3H), 8.49 (t, J=6.4 Hz, 1H), 7.99 (d, J=8.4 Hz, 1H), 7.91-7.67 (m, 2H), 7.42-6.75 (m, 9H), 6.24 (s, 1H), 4.30-4.15 (m, 4H), 3.87-3.78 (m, 4H), 3.65 (s, 6H), 3.31-3.24 (m, 2H), 2.91-2.88 (m, 2H), 2.41-2.35 (m, 3H), 2.04-1.98 (m, 4H), 1.02 (t, J=7.2 Hz, 3H). LCMS (ESI): m/z found 817.3[M+H]⁺.

### Compound 44

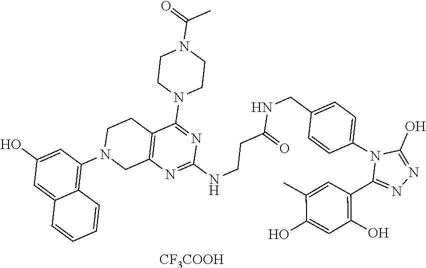 [SMILES: CC(=O)N1CCN(c2nc(NCCC(=O)NCc3ccc(-n4c(O)nnc4-c4cc(C)c(O)cc4O)cc3)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxy-5-methylphenyl)-5-hydroxy-4H-1,2,4-triazol-4-yl)benzyl)propanamide, trifluoroacetic acid

¹H NMR (400 MHz, DMSO-d₆): δ 11.86 (s, 1H), 9.77-9.32 (m, 3H), 8.49 (t, J=5.6 Hz, 1H), 8.00-7.68 (m, 3H), 7.42-6.75 (m, 9H), 6.23 (s, 1H), 4.28-4.15 (s, 4H), 3.96-3.82 (m, 4H), 3.77-3.60 (m, 9H), 3.24 (s, 2H), 2.96-2.85 (m, 2H), 2.04 (s, 3H), 1.95 (s, 3H). LCMS (ESI): m/z found 785.3[M−CF3COOH+H]⁺.

### Compound 45

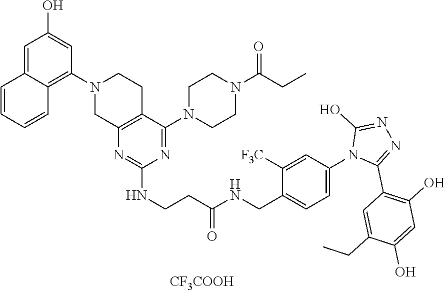 [SMILES: CCC(=O)N1CCN(c2nc(NCCC(=O)NCc3ccc(-n4c(O)nnc4-c4cc(CC)c(O)cc4O)cc3C(F)(F)F)nc3c2CCN(c2cc(O)cc4ccccc24)C3)CC1 ; "CF3COOH"]

### N-(4-(3-(5-ethyl-2,4-dihydroxyphenyl)-5-hydroxy-4H-1,2,4-triazol-4-yl)-2-(trifluoromethyl)benzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆): δ 12.01 (s, 1H), 9.82-9.30 (m, 3H), 8.58 (s, 1H), 7.98 (d, J=8.4 Hz, 1H), 7.74-6.72 (m, 11H), 6.23 (s, 1H), 4.47-4.39 (m, 2H), 4.14 (s, 2H), 3.94-3.78 (m, 4H), 3.63-3.58 (m, 5H), 3.28-3.21 (m, 2H), 2.96-2.84 (m, 2H), 2.59-2.54 (m, 2H), 2.42-2.32 (m, 5H), 1.06-0.98 (m, 6H). LCMS (ESI): m/z found 881.0[M−CF3COOH+H]⁺.

### Compound 46

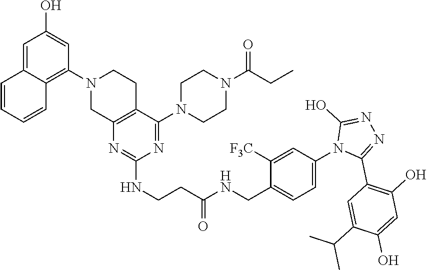 [SMILES: CCC(=O)N1CCN(c2nc(NCCC(=O)NCc3ccc(-n4c(O)nnc4-c4cc(C(C)C)c(O)cc4O)cc3C(F)(F)F)nc3c2CCN(c2cc(O)cc4ccccc24)C3)CC1]

### N-(4-(3-(2,4-dihydroxy-5-isopropylphenyl)-5-hydroxy-4H-1,2,4-triazol-4-yl)-2-(trifluoromethyl)benzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propenamide

¹H NMR (400 MHz, DMSO-d₆): δ 12.01 (s, 1H), 9.84-9.28 (m, 3H), 8.57 (s, 1H), 7.99 (d, J=8.0 Hz, 1H), 7.77-6.69 (m, 11H), 6.23 (s, 1H), 4.47-4.39 (m, 2H), 4.13 (s, 2H), 3.87-3.75 (m, 2H), 3.64-3.57 (m, 6H), 3.27-3.20 (m, 3H), 3.06-2.99 (m, 1H), 2.95-2.84 (m, 2H), 2.58-2.54 (m, 2H), 2.39-2.31 (m, 3H), 1.06-0.99 (m, 9H). LCMS (ESI): m/z found 895.0[M+H]⁺.

### Compound 47

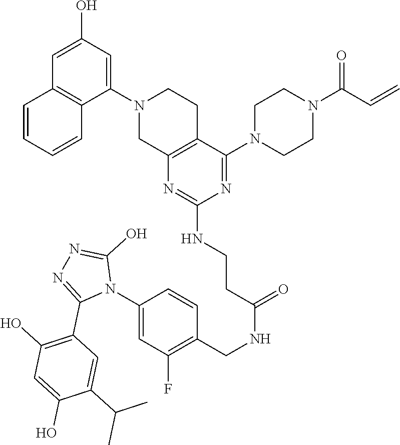 [SMILES: C=CC(=O)N1CCN(c2nc(NCCC(=O)NCc3ccc(-n4c(O)nnc4-c4cc(C(C)C)c(O)cc4O)cc3F)nc3c2CCN(c2cc(O)cc4ccccc24)C3)CC1]

### 3-((4-(4-acryloylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)-2-fluorobenzyl)propenamide

¹H NMR (400 MHz, DMSO-d₆): δ 11.96 (s, 1H), 9.82-9.34 (m, 3H), 8.50 (s, 1H), 8.06-7.57 (m, 3H), 7.45-6.74 (m, 10H), 6.28-6.12 (m, 2H), 5.77-5.71 (m, 1H), 4.32-4.26 (m, 2H), 4.16 (s, 2H), 3.93-3.83 (m, 4H), 3.78-3.68 (m, 4H), 3.63-3.56 (m, 3H), 3.28-3.21 (m, 2H), 3.07-2.88 (m, 3H), 1.03 (d, J=6.8 Hz, 6H). LCMS (ESI): m/z found 843.3[M+H]⁺.

### Compound 48

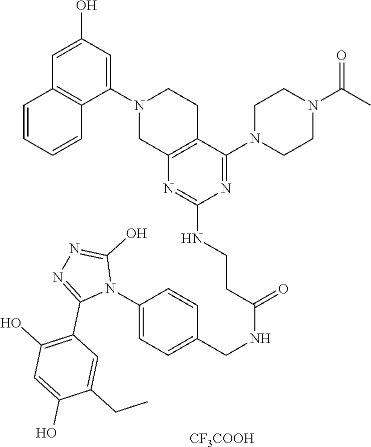 [SMILES: CCc1cc(-c2nnc(O)n2-c2ccc(CNC(=O)CCNc3nc4c(c(N5CCN(C(C)=O)CC5)n3)CCN(c3cc(O)cc5ccccc35)C4)cc2)c(O)cc1O ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-N-(4-(3-(5-ethyl-2,4-dihydroxyphenyl)-5-hydroxy-4H-1,2,4-triazol-4-yl)benzyl)propanamide, trifluoroacetic acid

¹H NMR (400 MHz, DMSO-d₆): δ 11.88 (s, 1H), 9.77 (s, 1H), 9.56 (s, 1H), 9.33 (s, 1H), 8.50 (t, J=6.0 Hz, 1H), 8.04-7.63 (m, 3H), 7.44-7.07 (m, 7H), 6.93-6.72 (m, 3H), 6.23 (s, 1H), 4.30-4.12 (m, 4H), 3.85 (d, J=22.4 Hz, 4H), 3.60 (s, 6H), 3.24 (s, 2H), 2.91 (s, 2H), 2.40-2.31 (m, 2H), 2.08-1.97 (m, 4H), 0.99 (t, J=7.6 Hz, 3H). LCMS (ESI): m/z found 799.3[M—CF3COOH+H]⁺.

### Compound 49

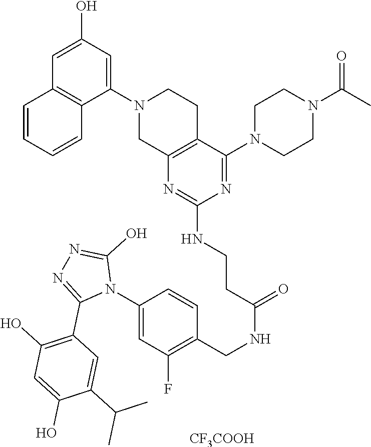 [SMILES: CC(=O)N1CCN(c2nc(NCCC(=O)NCc3ccc(-n4c(O)nnc4-c4cc(C(C)C)c(O)cc4O)cc3F)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)-2-fluorobenzyl)propanamide, trifluoroacetic acid

¹H NMR (400 MHz, DMSO-d₆): δ 11.96 (s, 1H), 9.84-9.28 (m, 3H), 8.50 (t, J=5.6 Hz, 1H), 8.06-7.62 (m, 3H), 7.48-6.67 (m, 9H), 6.25 (s, 1H), 4.29 (d, J=5.4 Hz, 2H), 4.16 (s, 2H), 3.85 (d, J=21.8 Hz, 4H), 3.60 (s, 6H), 3.24 (s, 2H), 3.05-2.87 (m, 3H), 2.10-1.96 (m, 4H), 1.03 (d, J=6.8 Hz, 6H). LCMS (ESI): m/z found 831.3[M—CF3COOH+H]⁺.

### Compound 50

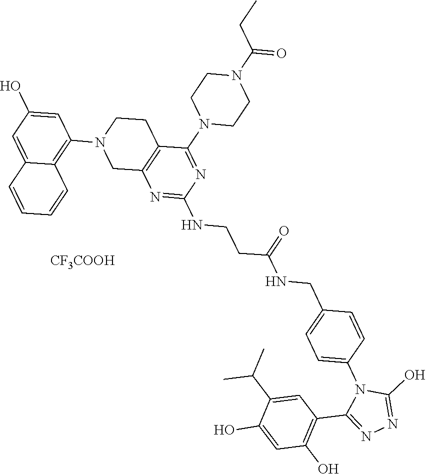 [SMILES: CCC(=O)N1CCN(c2nc(NCCC(=O)NCc3ccc(-n4c(O)nnc4-c4cc(C(C)C)c(O)cc4O)cc3)nc3c2CCN(c2cc(O)cc4ccccc24)C3)CC1 ; "CF3COOH"]

### N-(4-(3-(2,4-dihydroxy-5-isopropylphenyl)-5-hydroxy-4H-1,2,4-triazol-4-yl)benzyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆) δ 11.89 (s, 1H), 9.75 (s, 1H), 9.57 (s, 1H), 9.35 (s, 1H), 8.49 (s, 1H), 7.99 (d, J=8.4 Hz, 1H), 7.68 (d, J=8.1 Hz, 1H), 7.40 (t, J=7.7 Hz, 1H), 7.31-7.21 (m, 3H), 7.11 (d, J=8.3 Hz, 2H), 6.89 (s, 1H), 6.84 (s, 1H), 6.75 (s, 1H), 6.24 (s, 1H), 4.27 (d, J=5.6 Hz, 2H), 4.14 (s, 2H), 3.82 (s, 4H), 3.61 (s, 6H), 3.43 (s, 3H), 3.24 (s, 2H), 3.02-2.88 (m, 3H), 2.36 (dd, J=14.7, 7.4 Hz, 2H), 1.00 (m, 9H). LCMS (ESI): m/z found 827.1 [M−CF3COOH+H]⁺.

### Compound 51

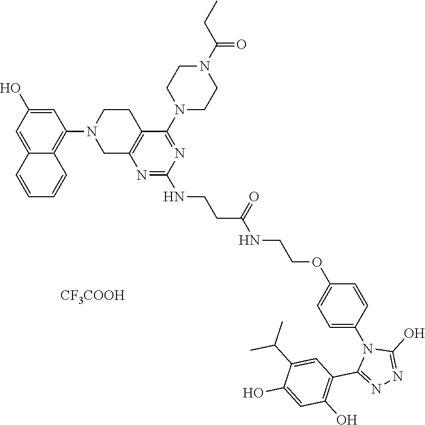 [SMILES: CCC(=O)N1CCN(c2nc(NCCC(=O)NCCOc3ccc(-n4c(O)nnc4-c4cc(C(C)C)c(O)cc4O)cc3)nc3c2CCN(c2cc(O)cc4ccccc24)C3)CC1 ; "CF3COOH"]

### N-(2-(4-(3-(2,4-dihydroxy-5-isopropylphenyl)-5-hydroxy-4H-1,2,4-triazol-4-yl)phenoxy)ethyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆) δ 11.86 (s, 1H), 9.74 (s, 1H), 9.57 (s, 1H), 9.37 (s, 1H), 8.23 (s, 1H), 7.98 (d, J=8.8 Hz, 1H), 7.68 (d, J=8.2 Hz, 1H), 7.40 (t, J=7.7 Hz, 1H), 7.30-7.25 (m, 1H), 7.10 (d, J=8.9 Hz, 2H), 6.95-6.88 (m, 3H), 6.80 (s, 1H), 6.75 (s, 1H), 6.25 (s, 1H), 4.15 (s, 2H), 3.96 (d, J=5.4 Hz, 2H), 3.83 (s, 4H), 3.62 (s, 8H), 3.24 (s, 3H), 3.01-2.88 (m, 3H), 2.46 (s, 1H), 2.36 (m, 3H), 1.07-0.91 (m, 9H). LCMS (ESI): m/z found 857.0 [M−CF3COOH+H]⁺.

### Compound 52

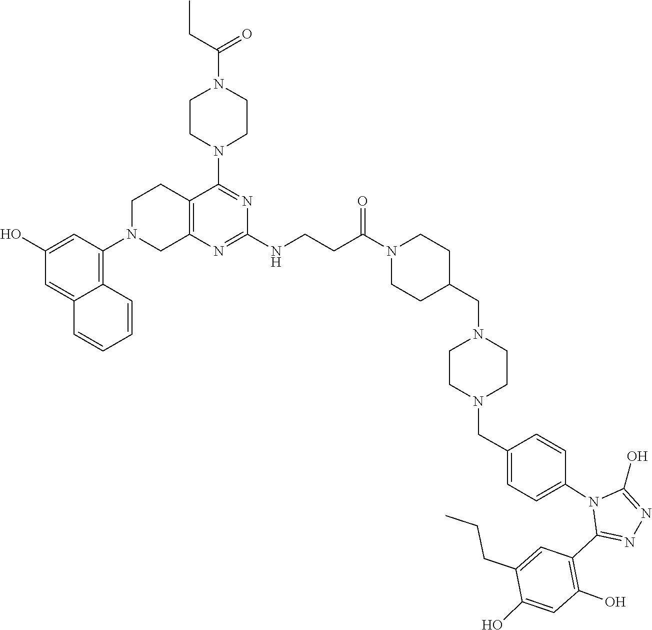

### 1-(4-((4-(4-(3-(2,4-dihydroxy-5-propylphenyl)-5-hydroxy-4H-1,2,4-triazol-4-yl)benzyl)piperazin-1-yl)methyl)piperidin-1-yl)-3-((7-(3-hydroxynaphthalen-1-yl)-4-(4-propionylpiperazin-1-yl)-5,6,7,8-tetrahydropyrido[3,4-d]pyrimidin-2-yl)amino)propan-1-one

¹H NMR (400 MHz, DMSO-d₆) δ 11.91 (s, 1H), 9.74 (s, 1H), 9.56 (s, 1H), 9.35 (s, 1H), 7.99 (d, J=8.2 Hz, 1H), 7.67 (d, J=8.5 Hz, 1H), 7.43-7.36 (m, 1H), 7.3-7.23 (m, 2H), 7.12 (s, 2H), 7.00 (s, 1H), 6.88 (s, 1H), 6.82 (s, 1H), 6.75 (s, 1H), 6.25 (s, 1H), 4.35 (s, 1H), 4.08 (s, 1H), 3.85 (s, 2H), 3.60 (s, 4H), 3.54-3.47 (m, 8H), 2.99 (s, 10H), 2.65 (m, 2H), 2.43-2.24 (m, 8H), 2.19-1.86 (m, 3H), 1.71 (s, 3H), 1.45-1.36 (m, 2H), 1.24 (s, 2H), 1.01 (t, J=7.2 Hz, 3H), 0.78 (t, J=7.3 Hz, 3H). LCMS (ESI): m/z found 993.5[M+H]⁺.

### Compound 53

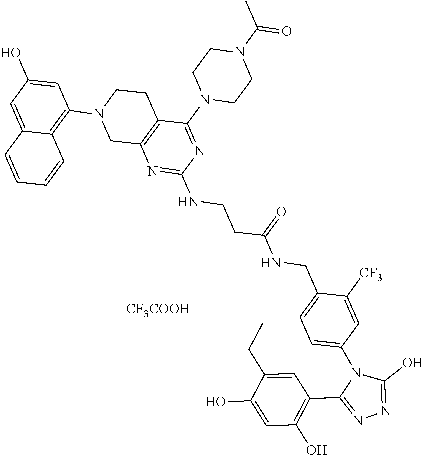 [SMILES: CCc1cc(-c2nnc(O)n2-c2ccc(CNC(=O)CCNc3nc4c(c(N5CCN(C(C)=O)CC5)n3)CCN(c3cc(O)cc5ccccc35)C4)c(C(F)(F)F)c2)c(O)cc1O ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-N-(4-(3-(5-ethyl-2,4-dihydroxyphenyl)-5-hydroxy-4H-1,2,4-triazol-4-yl)-2-(trifluoromethyl)benzyl)propanamide, trifluoroacetic acid

¹H NMR (400 MHz, DMSO-d₆) δ 12.01 (s, 1H), 9.74 (s, 1H), 9.59 (s, 1H), 9.36 (s, 1H), 8.57 (s, 1H), 7.99 (d, J=8.3 Hz, 1H), 7.68 (d, J=7.9 Hz, 1H), 7.53 (s, 1H), 7.43-7.36 (m, 3H), 7.28 (t, J=7.5 Hz, 1H), 6.98 (s, 1H), 6.89 (s, 1H), 6.75 (s, 1H), 6.23 (s, 1H), 4.42 (s, 2H), 4.14 (s, 2H), 3.83-3.68 (m, 4H), 3.60 (s, 8H), 3.24 (s, 2H), 2.90 (s, 2H), 2.41-2.34 (m, 2H), 2.04 (s, 3H), 1.23 (s, 1H), 1.02 (t, J=7.5 Hz, 3H). LCMS (ESI): m/z found 867.3[M−CF3COOH+H]⁺.

### Compound 54

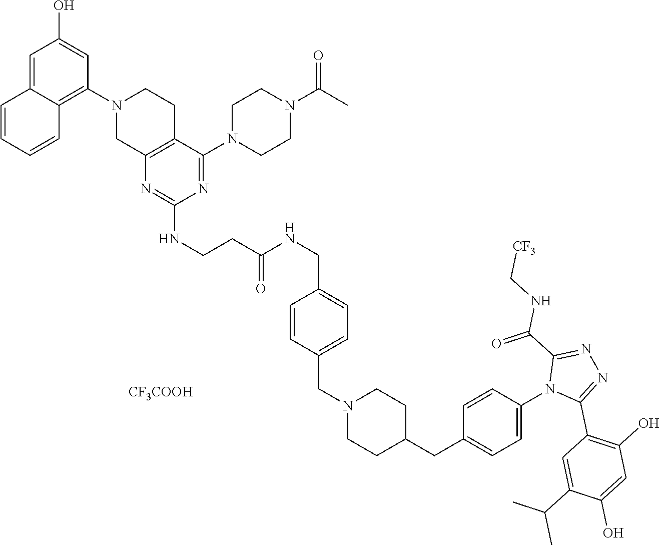 [SMILES: CC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; "CF3COOH"]

### 4-(4-((1-(4-((3-((4-(4-acetylpiperazin-1-yl)-7-(3-hydroxynaphthalen-1-yl)-5,6,7,8-tetrahydropyrido[3,4-d]pyrimidin-2-yl)amino)propanamido)methyl)benzyl)piperidin-4-yl)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆) δ 9.79 (s, 2H), 9.59 (d, J=6.1 Hz, 1H), 9.49 (s, 1H), 8.53 (s, 1H), 7.99 (d, J=8.3 Hz, 1H), 7.68 (d, J=7.9 Hz, 1H), 7.46-7.21 (m, 12H), 6.90 (s, 1H), 6.76 (s, 1H), 6.60 (s, 1H), 6.34 (s, 1H), 4.32 (d, J=5.4 Hz, 2H), 4.23 (s, 2H), 4.15 (s, 2H), 3.92-3.78 (m, 12H), 3.33 (d, J=10.2 Hz, 2H), 3.26 (s, 2H), 3.14 (s, 1H), 2.89-2.80 (m, 6H), 2.05 (s, 3H), 2.00 (d, J=7.6 Hz, 1H), 1.88-1.62 (m, 4H), 1.48-1.37 (m, 2H), 0.82 (d, J=6.9 Hz, 6H). LCMS (ESI): m/z found 1109.5[M−CF3COOH+H]⁺.

### Compound 55

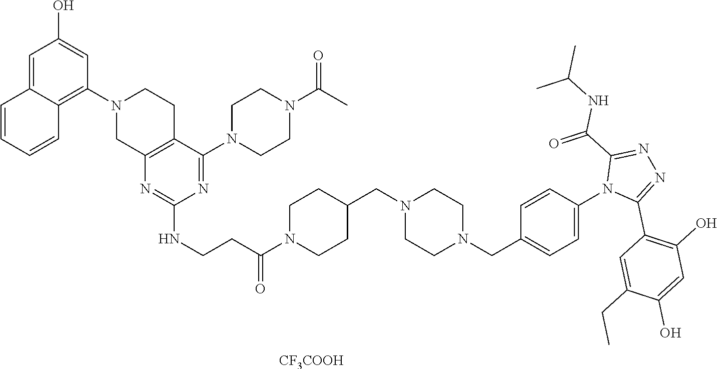 [SMILES: CCc1cc(-c2nnc(C(=O)NC(C)C)n2-c2ccc(CN3CCN(CC4CCN(C(=O)CCNc5nc6c(c(N7CCN(C(C)=O)CC7)n5)CCN(c5cc(O)cc7ccccc57)C6)CC4)CC3)cc2)c(O)cc1O ; "CF3COOH"]

### 4-(4-((4-((1-(3-((4-(4-acetylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(5-ethyl-2,4-dihydroxyphenyl)-N-isopropyl-4H-1,2,4-triazole-3-carboxamide, trifluoroacetic acid

¹H NMR (400 MHz, DMSO-d₆): δ 9.76 (s, 1H), 9.71 (s, 1H), 8.80 (d, J=8.0 Hz, 1H), 7.99 (d, J=8.0 Hz, 1H), 7.69 (d, J=8.0 Hz, 1H), 7.53-6.97 (m, 8H), 6.90 (s, 1H), 6.76 (s, 1H), 6.62 (s, 1H), 6.31 (s, 1H), 4.44-4.34 (m, 2H), 4.16 (s, 2H), 3.99-3.85 (m, 15H), 3.28-3.23 (m, 2H), 3.10-2.80 (m, 10H), 2.68-2.63 (m, 2H), 2.31-2.20 (m, 4H), 2.06 (s, 3H), 2.03-1.96 (m, 2H), 1.78-1.71 (m, 2H), 1.50-1.28 (m, 2H), 1.10 (d, J=6.4 Hz, 6H), 0.89-0.84 (m, 3H). LCMS (ESI): m/z found 1034.5[M—CF3COOH+H]⁺.

### Compound 56

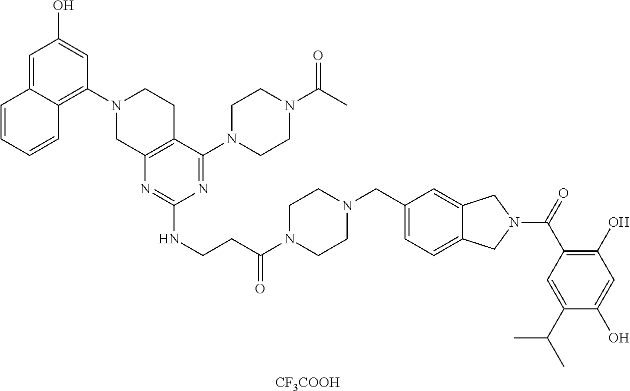 [SMILES: CC(=O)N1CCN(c2nc(NCCC(=O)N3CCN(Cc4ccc5c(c4)CN(C(=O)c4cc(C(C)C)c(O)cc4O)C5)CC3)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-1-(4-((2-(2,4-dihydroxy-5-isopropylbenzoyl)isoindolin-5-yl)methyl)piperazin-1-yl)propan-1-one, trifluoroacetic acid

¹H NMR (400 MHz, DMSO-d₆): δ 10.02 (s, 1H), 9.75 (s, 1H), 9.64 (s, 1H), 7.99 (d, J=8.0 Hz, 1H), 7.68 (d, J=8.0 Hz, 1H), 7.55-7.23 (m, 6H), 7.03 (s, 1H), 6.89 (s, 1H), 6.75 (s, 1H), 6.40 (s, 1H), 4.87-4.75 (m, 4H), 4.54-4.28 (m, 3H), 4.14 (s, 2H), 3.95-3.60 (m, 14H), 3.27-3.24 (m, 2H), 3.13-3.07 (m, 2H), 2.97-2.85 (m, 4H), 2.70-2.66 (m, 2H), 2.05 (s, 3H), 1.13 (d, J=6.8 Hz, 6H). LCMS (ESI): m/z found 868.4[M−CF3COOH+H]⁺.

### Compound 57

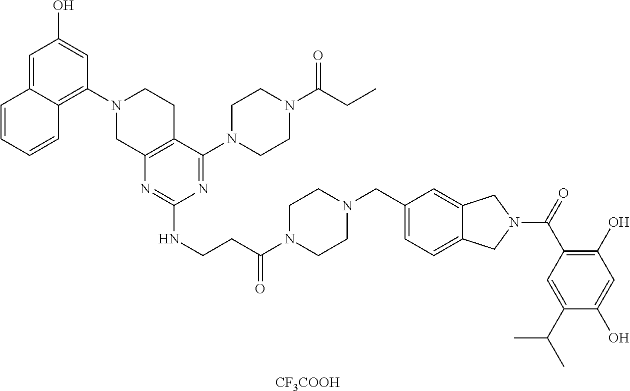 [SMILES: CCC(=O)N1CCN(c2nc(NCCC(=O)N3CCN(Cc4ccc5c(c4)CN(C(=O)c4cc(C(C)C)c(O)cc4O)C5)CC3)nc3c2CCN(c2cc(O)cc4ccccc24)C3)CC1 ; "CF3COOH"]

### 1-(4-((2-(2,4-dihydroxy-5-isopropylbenzoyl)isoindolin-5-yl)methyl)piperazin-1-yl)-3-((7-(3-hydroxynaphthalen-1-yl)-4-(4-propionylpiperazin-1-yl)-5,6,7,8-tetrahydropyrido[3,4-d]pyrimidin-2-yl)amino)propan-1-one, trifluoroacetic acid

¹H NMR (400 MHz, DMSO-d₆): δ 10.01 (s, 1H), 9.75 (s, 1H), 9.64 (s, 1H), 7.99 (d, J=8.0 Hz, 1H), 7.68 (d, J=8.4 Hz, 1H), 7.54-7.23 (m, 6H), 7.03 (s, 1H), 6.89 (s, 1H), 6.75 (s, 1H), 6.40 (s, 1H), 4.86-4.76 (m, 4H), 4.52-4.30 (m, 3H), 4.15 (s, 2H), 4.00-3.74 (m, 10H), 3.40-3.30 (m, 4H), 3.27-3.22 (m, 2H), 3.13-3.05 (m, 2H), 3.00-2.84 (m, 4H), 2.71-2.65 (m, 2H), 2.40-2.34 (m, 2H), 1.13 (d, J=6.8 Hz, 6H), 1.05-0.99 (m, 3H). LCMS (ESI): m/z found 882.4[M—CF3COOH+H]⁺.

### Compound 58

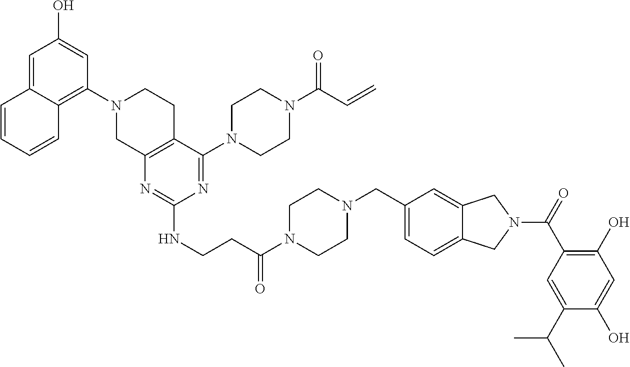 [SMILES: C=CC(=O)N1CCN(c2nc(NCCC(=O)N3CCN(Cc4ccc5c(c4)CN(C(=O)c4cc(C(C)C)c(O)cc4O)C5)CC3)nc3c2CCN(c2cc(O)cc4ccccc24)C3)CC1]

### 1-(4-(2-((3-(4-((2-(2,4-dihydroxy-5-isopropylbenzoyl)isoindolin-5-yl)methyl)piperazin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one

¹H NMR (400 MHz, DMSO-d₆): δ 10.25-9.90 (m, 2H), 9.78 (s, 1H), 9.66 (s, 1H), 7.99 (d, J=8.8 Hz, 1H), 7.69 (d, J=8.1 Hz, 1H), 7.55-7.20 (m, 6H), 7.03 (s, 1H), 6.89 (s, 1H), 6.88-6.79 (m, 1H), 6.75 (s, 1H), 6.41 (s, 1H), 6.17 (dd, J=16.3, 1.9 Hz, 1H), 5.75 (dd, J=10.5, 1.8 Hz, 1H), 4.88-4.72 (m, 4H), 4.52-4.26 (m, 3H), 4.14 (s, 2H), 4.06-3.64 (m, 14H), 3.26 (s, 2H), 3.13-3.05 (m, 2H), 2.98-2.84 (m, 4H), 2.72-2.66 (m, 2H), 1.13 (d, J=6.8 Hz, 6H). LCMS (ESI): S m/z found 880.4[M+H]⁺.

### Compound 59

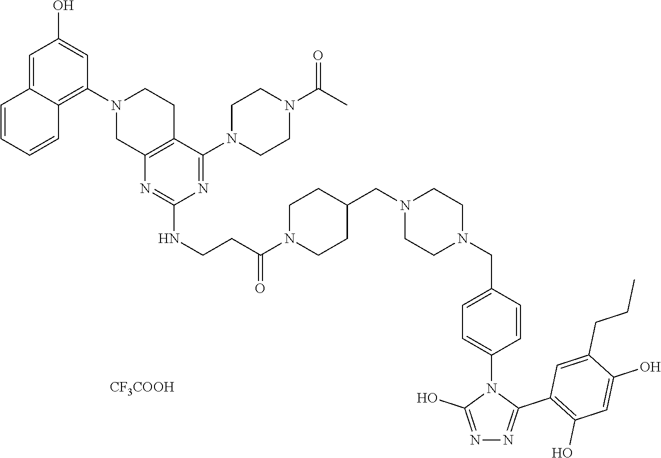 [SMILES: CCCc1cc(-c2nnc(O)n2-c2ccc(CN3CCN(CC4CCN(C(=O)CCNc5nc6c(c(N7CCN(C(C)=O)CC7)n5)CCN(c5cc(O)cc7ccccc57)C6)CC4)CC3)cc2)c(O)cc1O ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-1-(4-((4-(4-(3-(2,4-dihydroxy-5-propylphenyl)-5-hydroxy-4H-1,2,4-triazol-4-yl)benzyl)piperazin-1-yl)methyl)piperidin-1-yl)propan-1-one, trifluoroacetic acid

¹H NMR (400 MHz, DMSO-d₆): δ 9.89-9.18 (m, 3H), 8.00 (d, J=6.8 Hz, 1H), 7.66 (d, J=6.8 Hz, 1H), 7.48-7.02 (m, 7H), 6.92-6.70 (m, 3H), 6.62-6.44 (m, 1H), 6.25 (s, 1H), 4.36 (d, J=12.4 Hz, 1H), 4.05-3.82 (m, 3H), 3.56 (s, 6H), 3.02-2.87 (m, 3H), 2.85-2.60 (m, 6H), 2.40-2.23 (m, 10H), 2.05 (s, 7H), 1.79-1.57 (m, 4H), 1.42-1.31 (m, 2H), 1.00-0.84 (m, 2H), 0.80-0.72 (m, 3H). LCMS (ESI): m/z found 979.5[M−CF3COOH+H]⁺.

### Compound 60

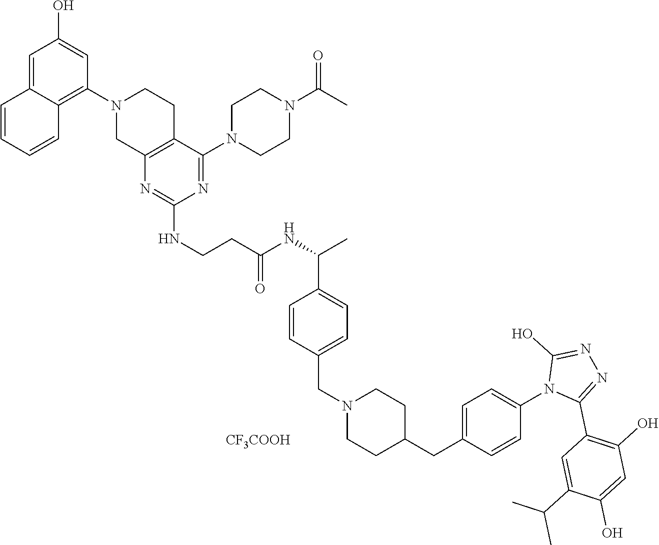 [SMILES: CC(=O)N1CCN(c2nc(NCCC(=O)N[C@H](C)c3ccc(CN4CCC(Cc5ccc(-n6c(O)nnc6-c6cc(C(C)C)c(O)cc6O)cc5)CC4)cc3)nc3c2CCN(c2cc(O)cc4ccccc24)C3)CC1 ; "CF3COOH"]

### (R)-3-((4-(4-acetylpiperazin-1-yl)-7-(3-hydroxynaphthalen-1-yl)-5,6,7,8-tetrahydropyrido[3,4-d]pyrimidin-2-yl)amino)-N-(1-(4-((4-(4-(3-(2,4-dihydroxy-5-isopropylphenyl)-5-hydroxy-4H-1,2,4-triazol-4-yl)benzyl)piperidin-1-yl)methyl)phenyl)ethyl)propanamide, trifluoroacetic acid

¹H NMR (400 MHz, DMSO-d₆). δ 11.92 (s, 1H), 9.77 (s, 1H), 9.61 (s, 1H), 9.40 (s, 2H), 8.47 (s, 1H), 7.99 (d, J=8.0 Hz, 1H), 7.68 (d, J=8.6 Hz, 1H), 7.52-7.34 (m, 5H), 7.31-7.25 (m, 1H), 7.21-7.14 (m, 2H), 7.10 (d, J=8.4 Hz, 2H), 6.89 (s, 1H), 6.78-6.72 (m, 2H), 6.26 (s, 1H), 5.02-4.92 (m, 1H), 4.24-4.17 (m, 2H), 4.13 (s, 2H), 3.92-3.60 (m, 10H), 3.34-3.08 (m, 8H), 3.03-2.77 (m, 6H), 2.05 (s, 3H), 1.77-1.66 (m, 3H), 1.43-1.29 (m, 5H), 0.94 (d, J=6.8 Hz, 6H). LCMS (ESI): m/z found 1014.5[M−CF3COOH+H]⁺.

### Compound 61

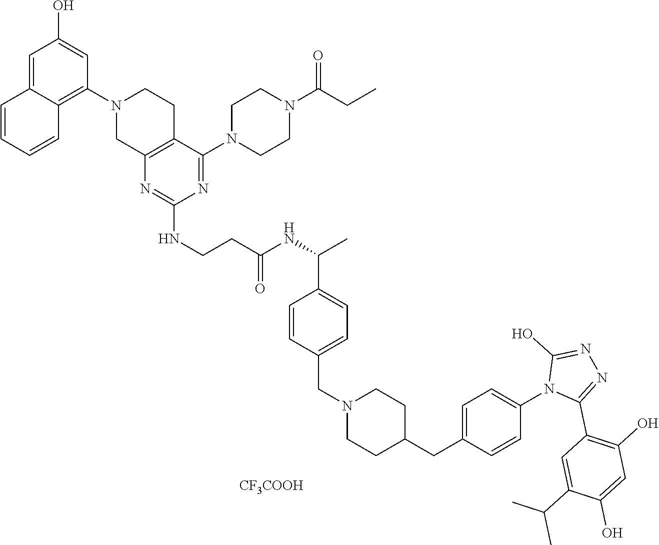 [SMILES: CCC(=O)N1CCN(c2nc(NCCC(=O)N[C@H](C)c3ccc(CN4CCC(Cc5ccc(-n6c(O)nnc6-c6cc(C(C)C)c(O)cc6O)cc5)CC4)cc3)nc3c2CCN(c2cc(O)cc4ccccc24)C3)CC1 ; "CF3COOH"]

### (R)—N-(1-(4-(4-(4-(3-(2,4-dihydroxy-5-isopropylphenyl)-5-hydroxy-4H-1,2,4-triazol-4-yl)benzyl)piperidin-1-yl)methyl)phenyl)ethyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆) δ 11.92 (s, 1H), 9.77 (s, 1H), 9.61 (s, 1H), 9.40 (s, 1H), 9.36-9.28 (m, 1H), 8.46 (s, 1H), 7.99 (d, J=8.3 Hz, 1H), 7.68 (d, J=8.1 Hz, 1H), 7.38 (t, J 6.9 Hz, 5H), 7.30-7.26 (m, 1H), 7.17 (d, J=8.2 Hz, 2H), 7.10 (d, J=8.3 Hz, 2H), 6.89 (s, 1H), 6.75 (d, J=5.7 Hz, 2H), 6.26 (s, 1H), 4.97 (d, J=7.3 Hz, 1H), 4.20 (s, 2H), 4.12 (s, 2H), 3.66 (m, 9H), 3.32 (s, 6H), 3.17-3.06 (m, 2H), 2.93 (m, 6H), 2.36 (d, J=7.5 Hz, 2H), 1.72 (d, J=11.5 Hz, 3H), 1.34 (d, J=6.8 Hz, 5H), 1.01 (t, J=7.3 Hz, 3H), 0.94 (d, J=6.8 Hz, 6H). LCMS (ESI): m/z found 1028.6 [M—CF3COOH+H]⁺.

### Compound 62

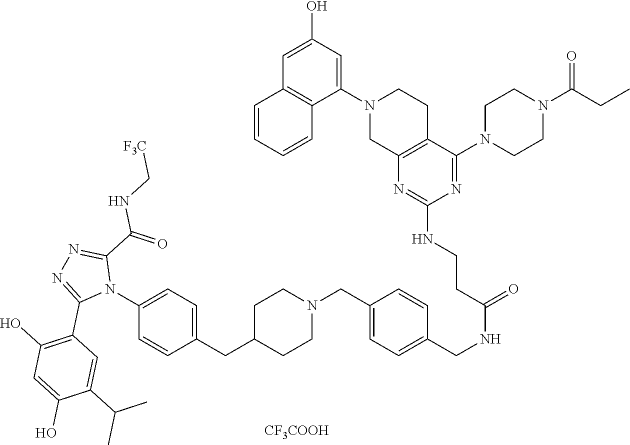 [SMILES: CCC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; "CF3COOH"]

### 5-(2,4-dihydroxy-5-isopropylphenyl)-4-(4-((1-(4-((3-((7-(3-hydroxynaphthalen-1-yl)-4-(4-propionylpiperazin-1-yl)-5,6,7,8-tetrahydropyrido[3,4-d]pyrimidin-2-yl)amino)propanamido)methyl)benzyl)piperidin-4-yl)methyl)phenyl)-N-(2,2,2-trifluoroethyl)-4H-1,2,4-triazole-3-carboxamide, trifluoroacetic acid

¹H NMR (400 MHz, DMSO-d₆) δ 12.28 (s, 1H), 9.80 (s, 1H), 9.60 (t, J=6.3 Hz, 1H), 9.29 (s, 1H), 8.54 (s, 1H), 7.96-7.95 (m, 1H), 7.67 (d, J=8.3 Hz, 2H), 7.42-7.41 (m, 3H), 7.28 (m, 7H), 6.88 (s, 1H), 6.73 (s, 1H), 6.58 (s, 1H), 6.32 (s, 1H), 4.25-4.23 (m, 7H), 3.91 (m, 7H), 3.25 (m, 6H), 2.97-2.77 (m, 5H), 2.70 (t, J=16.4 Hz, 1H), 2.53 (d, J=5.7 Hz, 3H), 2.34 (q, J=7.4 Hz, 2H), 2.02-2.01 (m, 1H), 1.74-1.73 (m, 3H), 1.37 (d, J=13.0 Hz, 2H), 1.21 (s, 1H), 0.99 (t, J=7.3 Hz, 3H), 0.80 (d, J=6.8 Hz, 6H). LCMS (ESI): m/z found 1123.6 [M—CF3COOH+H]⁺.

### Compound 63

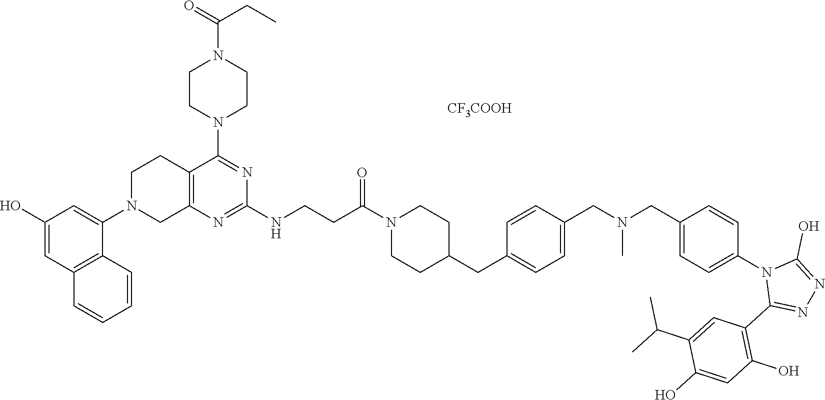 [SMILES: CCC(=O)N1CCN(c2nc(NCCC(=O)N3CCC(Cc4ccc(CN(C)Cc5ccc(-n6c(O)nnc6-c6cc(C(C)C)c(O)cc6O)cc5)cc4)CC3)nc3c2CCN(c2cc(O)cc4ccccc24)C3)CC1 ; "CF3COOH"]

### (R)-3-((4-(4-acetylpiperazin-1-yl)-7-(3-hydroxynaphthalen-1-yl)-5,6,7,8-tetrahydropyrido[3,4-d]pyrimidin-2-yl)amino)-N-(1-(4-((4-(4-(3-(2,4-dihydroxy-5-isopropylphenyl)-5-hydroxy-4H-1,2,4-triazol-4-yl)benzyl)piperidin-1-yl)methyl)phenyl)ethyl)propanamide, trifluoroacetic acid

¹H NMR (400 MHz, DMSO-d₆): δ 11.98 (s, 1H), 9.75-9.37 (m, 4H), 7.99 (d, J=8.4 Hz, 1H), 7.69 (d, J=8.0 Hz, 1H), 7.51-7.25 (m, 12H), 6.89 (d, J=3.6 Hz, 2H), 6.75 (s, 1H), 6.24 (s, 1H), 4.45-4.35 (m, 3H), 4.17 (s, 4H), 3.93-3.72 (m, 5H), 3.63 (s, 7H), 3.25 (s, 2H), 3.10-2.77 (m, 5H), 2.64 (s, 2H), 2.41-2.31 (m, 2H), 1.82-1.50 (m, 3H), 1.24 (s, 3H), 1.16-0.88 (m, 11H). LCMS (ESI): m/z found 1028.5[M−CF3COOH+H]⁺.

### Compound 64

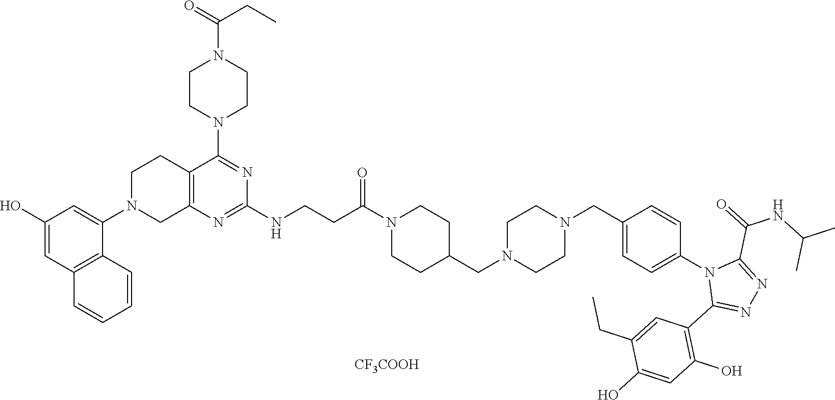 [SMILES: CCC(=O)N1CCN(c2nc(NCCC(=O)N3CCC(CN4CCN(Cc5ccc(-n6c(C(=O)NC(C)C)nnc6-c6cc(CC)c(O)cc6O)cc5)CC4)CC3)nc3c2CCN(c2cc(O)cc4ccccc24)C3)CC1 ; "CF3COOH"]

### 5-(5-ethyl-2,4-dihydroxyphenyl)-4-(4-((4-((1-(3-((7-(3-hydroxynaphthalen-1-yl)-4-(4-propionylpiperazin-1-yl)-5,6,7,8-tetrahydropyrido[3,4-d]pyrimidin-2-yl)amino)propanoyl)piperidin-4-yl)methyl)piperazin-1-yl)methyl)phenyl)-N-isopropyl-4H-1,2,4-triazole-3-carboxamide, trifluoroacetic acid

¹H NMR (400 MHz, DMSO-d₆) δ 9.74 (s, 2H), 8.80 (d, J=8.1 Hz, 1H), 7.99 (d, J=7.8 Hz, 1H), 7.90-7.62 (m, 2H), 7.51-7.22 (m, 8H), 6.90 (s, 1H), 6.76 (s, 1H), 6.64 (s, 1H), 6.31 (s, 1H), 4.39 (d, J=10.8 Hz, 1H), 4.17 (s, 3H), 3.94-3.82 (m, 9H), 3.26 (s, 4H), 3.03-2.81 (m, 11H), 2.40-2.20 (m, 5H), 2.08-1.91 (m, 2H), 1.86-1.65 (m, 3H), 1.12-0.83 (m, 18H). LCMS (ESI): m/z found 1048.6[M—CF3COOH+H]⁺.

### Compound 65

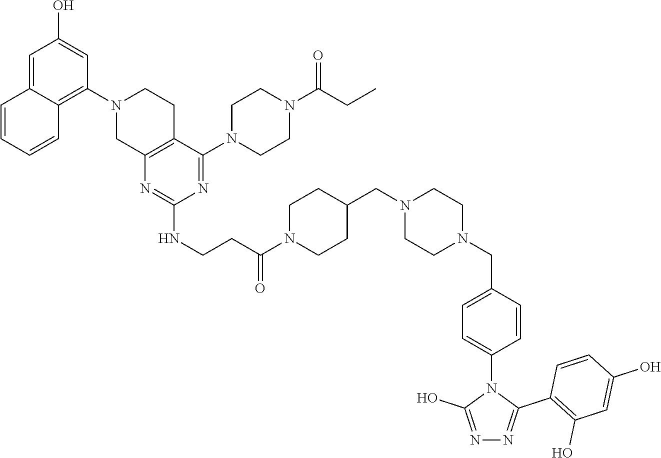 [SMILES: CCC(=O)N1CCN(c2nc(NCCC(=O)N3CCC(CN4CCN(Cc5ccc(-n6c(O)nnc6-c6ccc(O)cc6O)cc5)CC4)CC3)nc3c2CCN(c2cc(O)cc4ccccc24)C3)CC1]

### 1-(4-((4-(4-(3-(2,4-dihydroxyphenyl)-5-hydroxy-4H-1,2,4-triazol-4-yl)benzyl)piperazin-1-yl)methyl)piperidin-1-yl)-3-((7-(3-hydroxynaphthalen-1-yl)-4-(4-propionylpiperazin-1-yl)-5,6,7,8-tetrahydropyrido[3,4-d]pyrimidin-2-yl)amino)propan-1-one, trifluoroacetic acid

¹H NMR (400 MHz, DMSO-d₆) δ 9.67 (s, 2H), 8.00 (s, 1H), 7.66 (s, 1H), 7.39 (s, 1H), 7.32-7.19 (m, 4H), 7.13-7.03 (m, 3H), 6.85 (s, 1H), 6.75 (s, 1H), 6.50 (s, 1H), 6.25-6.14 (m, 2H), 5.34 (s, 1H), 4.61 (s, 2H), 4.38-4.34 (m, 1H), 3.98 (s, 3H), 3.53 (s, 6H), 2.94-2.67 (m, 10H), 2.38-2.27 (m, 10H), 2.05-1.94 (m, 4H), 1.69-1.63 (m, 3H), 1.51-1.43 (m, 2H), 1.02-0.99 (m, 3H). LCMS (ESI): m/z found 951.6[M+H]⁺.

### Compound 66

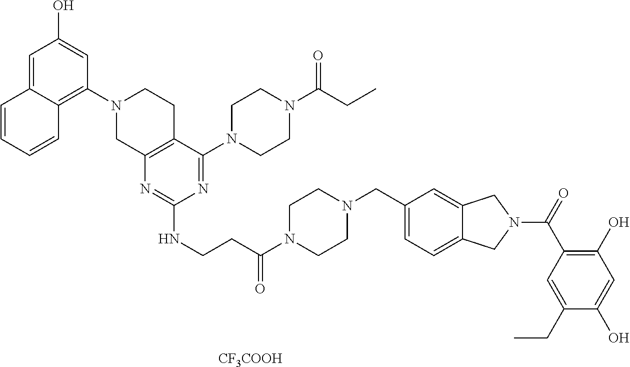 [SMILES: CCC(=O)N1CCN(c2nc(NCCC(=O)N3CCN(Cc4ccc5c(c4)CN(C(=O)c4cc(CC)c(O)cc4O)C5)CC3)nc3c2CCN(c2cc(O)cc4ccccc24)C3)CC1 ; "CF3COOH"]

### 1-(4-((2-(5-ethyl-2,4-dihydroxybenzoyl)isoindolin-5-yl)methyl)piperazin-1-yl)-3-((7-(3-hydroxynaphthalen-1-yl)-4-(4-propionylpiperazin-1-yl)-5,6,7,8-tetrahydropyrido[3,4-d]pyrimidin-2-yl)amino)propan-1-one, trifluoroacetic acid

¹H NMR (400 MHz, DMSO-d₆): δ 10.10 (s, 1H), 9.79-9.61 (m, 2H), 7.99 (d, J=8.4 Hz, 1H), 7.68 (d, J=8.0 Hz, 1H), 7.52-7.21 (m, 5H), 7.05-6.84 (m, 2H), 6.75 (s, 1H), 6.40 (s, 1H), 4.88-4.74 (m, 4H), 4.50-4.26 (m, 2H), 4.18-4.08 (m, 2H), 3.92-3.72 (m, 4H), 3.68-3.52 (m, 11H), 3.27-3.22 (m, 2H), 3.00-2.81 (m, 5H), 2.70-2.64 (m, 2H), 2.47-2.42 (m, 2H), 2.40-2.30 (m, 3H), 2.08-1.96 (m, 1H), 1.12-1.07 (m, 3H), 1.01 (t, J=7.2 Hz, 3H). LCMS (ESI): m/z found 868.4[M−CF3COOH+H]⁺.

### Compound 67

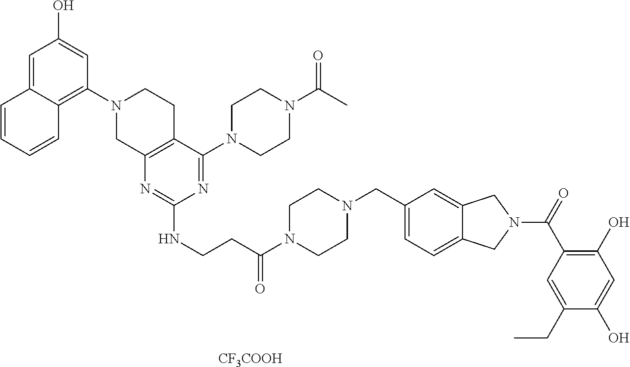 [SMILES: CCc1cc(C(=O)N2Cc3ccc(CN4CCN(C(=O)CCNc5nc6c(c(N7CCN(C(C)=O)CC7)n5)CCN(c5cc(O)cc7ccccc57)C6)CC4)cc3C2)c(O)cc1O ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-1-(4-((2-(5-ethyl-2,4-dihydroxybenzoyl)isoindolin-5-yl)methyl)piperazin-1-yl)propan-1-one, trifluoroacetic acid

¹H NMR (400 MHz, DMSO-d₆): δ 10.41-9.94 (m, 2H), 9.82-9.63 (m, 2H), 7.99 (d, J=8.8 Hz, 1H), 7.69 (d, J=8.0 Hz, 1H), 7.56-7.22 (m, 6H), 7.05-6.70 (m 3H), 6.41 (s, 1H), 4.88-4.74 (m, 4H), 4.55-4.30 (m, 3H), 4.22-4.08 (m, 3H), 3.94-3.79 (m, 4H), 3.63-3.58 (m, 7H), 3.28-3.21 (m, 3H), 3.00-2.82 (m, 6H), 2.7-2.64 (m, 3H), 2.47-2.41 (m, 3H), 2.05 (s, 4H), 1.09 (t, J=7.6 Hz, 3H). LCMS (ESI): m/z found 854.4[M−CF3COOH+H]⁺.

### Compound 68

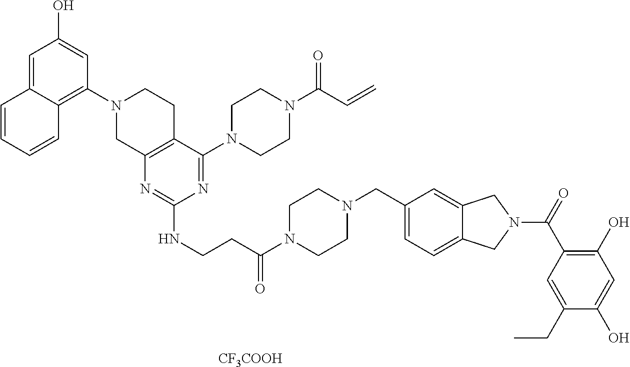 [SMILES: C=CC(=O)N1CCN(c2nc(NCCC(=O)N3CCN(Cc4ccc5c(c4)CN(C(=O)c4cc(CC)c(O)cc4O)C5)CC3)nc3c2CCN(c2cc(O)cc4ccccc24)C3)CC1 ; "CF3COOH"]

### 1-(4-(2-((3-(4-((2-(5-ethyl-2,4-dihydroxybenzoyl)isoindolin-5-yl)methyl)piperazin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 10.10 (s, 1H), 9.80-9.61 (m, 2H), 7.99 (d, J=8.0 Hz, 1H), 7.68 (d, J=8.4 Hz, 1H), 7.52-7.22 (m, 5H), 7.06-6.73 (m, 4H), 6.41 (s, 1H), 6.21-6.13 (m, 1H), 5.78-5.72 (m, 1H), 4.88-4.74 (m, 4H), 4.46-4.28 (m, 2H), 4.19-4.09 (m, 2H), 3.90-3.68 (m 10H), 3.42-3.33 (m, 4H), 3.29-3.20 (m, 3H), 2.98-2.83 (m, 4H), 2.74-2.62 (m, 3H), 2.47-2.42 (m, 2H), 1.10 (t, J=7.6 Hz, 3H). LCMS (ESI): m/z found 866.4[M−CF3COOH+H]⁺.

### Compound 69

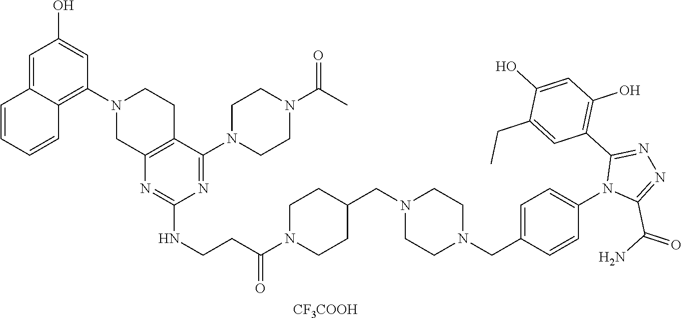 [SMILES: CCc1cc(-c2nnc(C(N)=O)n2-c2ccc(CN3CCN(CC4CCN(C(=O)CCNc5nc6c(c(N7CCN(C(C)=O)CC7)n5)CCN(c5cc(O)cc7ccccc57)C6)CC4)CC3)cc2)c(O)cc1O ; "CF3COOH"]

### 4-(4-((4-((1-(3-((4-(4-acetylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(5-ethyl-2,4-dihydroxyphenyl)-4H-1,2,4-triazole-3-carboxamide, trifluoroacetic acid

¹H NMR (400 MHz, DMSO-d₆): δ 9.82-9.65 (m, 2H), 8.32 (s, 1H), 7.99 (d, J=8.8 Hz, 1H), 7.78-7.62 (m, 3H), 7.46-7.23 (m, 7H), 6.90-6.59 (m, 3H), 6.31 (s, 1H), 4.39 (d, J=12.3 Hz, 2H), 4.17 (s, 3H), 3.94-3.79 (m, 6H), 3.62 (s, 8H), 3.25 (s, 2H), 3.08-2.87 (m, 7H), 2.67-2.64 (m, 1H), 2.34-2.21 (m, 5H), 2.06 (s, 4H), 1.75 (d, J=14.9 Hz, 3H), 1.13-0.97 (m, 2H), 0.86 (t, J=7.4 Hz, 4H). LCMS (ESI): m/z found 992.5[M−CF3COOH+H]⁺.

### Compound 70

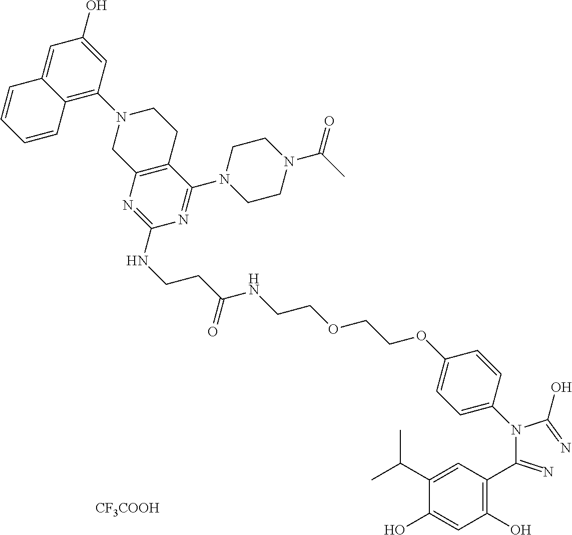 [SMILES: CC(=O)N1CCN(c2nc(NCCC(=O)NCCOCCOc3ccc(N(C(=N)O)C(=N)c4cc(C(C)C)c(O)cc4O)cc3)nc3c2CCN(c2cc(O)cc4ccccc24)C3)CC1 ; "CF3COOH"]

### 3-((4-(4-acetylpiperazin-1-yl)-7-(3-hydroxynaphthalen-1-yl)-5,6,7,8-tetrahydropyrido[3,4-d]pyrimidin-2-yl)amino)-N-(2-(2-(4-(3-(2,4-dihydroxy-5-isopropylphenyl)-5-hydroxy-4H-1,2,4-triazol-4-yl)phenoxy)ethoxy)ethyl)propanamide, trifluoroacetic acid

¹H NMR (400 MHz, DMSO-d₆): δ 11.87 (s, 1H), 9.75 (s, 1H), 9.58 (s, 1H), 9.38 (s, 1H), 8.15-7.93 (m, 2H), 7.68 (d, J=8.2 Hz, 1H), 7.42-7.26 (m, 2H), 7.14-6.71 (m, 7H), 6.25 (s, 1H), 4.19-4.03 (m, 4H), 3.97-3.77 (m, 4H), 3.73-3.69 (m, 2H), 3.65-3.58 (m, 4H), 3.50-3.45 (m, 5H), 3.29-3.20 (m, 4H), 3.02-2.87 (m, 3H), 2.45-2.40 (m, 2H), 2.06 (d, J=10.8 Hz, 3H), 0.97 (d, J=6.8 Hz, 6H). LCMS (ESI): m/z found 887.4[M—CF3COOH+H]⁺.

### Compound 71

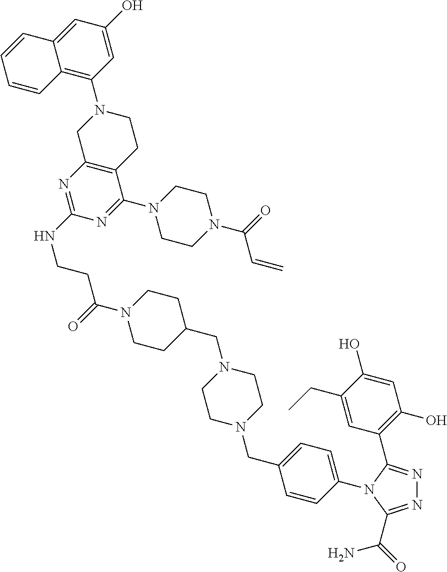 [SMILES: C=CC(=O)N1CCN(c2nc(NCCC(=O)N3CCC(CN4CCN(Cc5ccc(-n6c(C(N)=O)nnc6-c6cc(CC)c(O)cc6O)cc5)CC4)CC3)nc3c2CCN(c2cc(O)cc4ccccc24)C3)CC1]

### 4-(4-((4-((1-(3-((4-(4-acryloylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(5-ethyl-2,4-dihydroxyphenyl)-4H-1,2,4-triazole-3-carboxamide

¹H NMR (400 MHz, DMSO-d₆): δ 9.74 (d, J=23.0 Hz, 2H), 8.31 (s, 1H), 7.99 (d, J=9.2 Hz, 1H), 7.76-7.64 (m, 3H), 7.45-7.25 m, 6H), 6.93-6.56 (m, 4H), 6.34-6.12 (m, 2H), 5.75 (d, J=10.4 Hz, 1H), 4.46-4.29 (m, 3H), 4.15 (s, 3H), 3.78-3.70 (m, 10H), 3.28-3.21 (s, 3H), 3.06-2.88 (m, 10H), 2.72-2.62 (m, 5H), 2.29-2.20 (m, 2H), 1.82-1.69 (m, 2H), 1.14-0.98 (m, 2H), 0.93-0.79 (m, 4H). LCMS (ESI): m/z found 1004.4[M+H]⁺.

### Compound 72

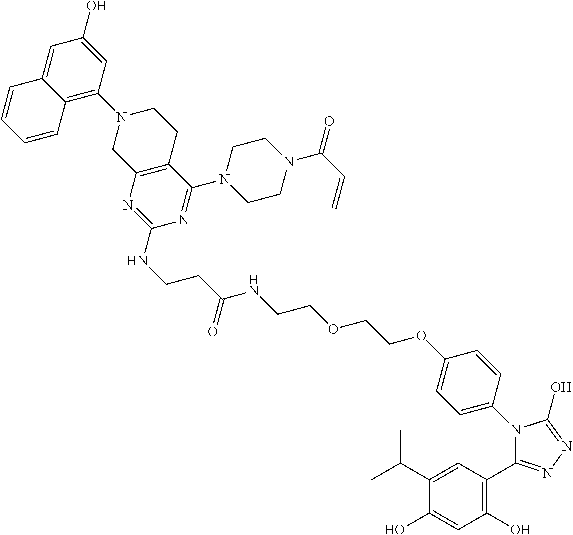 [SMILES: C=CC(=O)N1CCN(c2nc(NCCC(=O)NCCOCCOc3ccc(-n4c(O)nnc4-c4cc(C(C)C)c(O)cc4O)cc3)nc3c2CCN(c2cc(O)cc4ccccc24)C3)CC1]

### (R)-3-((4-(4-acetylpiperazin-1-yl)-7-(3-hydroxynaphthalen-1-yl)-5,6,7,8-tetrahydropyrido[3,4-d]pyrimidin-2-yl)amino)-N-(1-(4-((4-(4-(3-(2,4-dihydroxy-5-isopropylphenyl)-5-hydroxy-4H-1,2,4-triazol-4-yl)benzyl)piperidin-1-yl)methyl)phenyl)ethyl)propanamide, trifluoroacetic acid

¹H NMR (400 MHz, DMSO-d₆) δ 11.86 (s, 1H), 9.75 (s, 1H), 9.58 (s, 1H), 9.38 (s, 1H), 8.07 (s, 1H), 7.99 (d, J=8.7 Hz, 1H), 7.68 (d, J=8.4 Hz, 1H), 7.40 (t, J=7.5 Hz, 1H), 7.32-7.24 (m, 1H), 7.09 (d, J=8.9 Hz, 2H), 6.92 (m, 3H), 6.84-6.74 (m, 3H), 6.25 (s, 1H), 6.17-6.15 (m, 1H), 5.74 (d, J=12.6 Hz, 1H), 4.15 (s, 2H), 4.07 (s, 2H), 3.87 (s, 3H), 3.73-3.71 (m, 6H), 3.48 (d, J=5.8 Hz, 5H), 3.25 (d, J=5.7 Hz, 4H), 2.96-2.93 (m, 3H), 2.45-2.43 (m, 2H), 2.08 (d, J=4.4 Hz, 1H), 0.97 (d, J=6.9 Hz, 6H). LCMS (ESI): m/z found 899.6[M+H]⁺.

### Compound 73

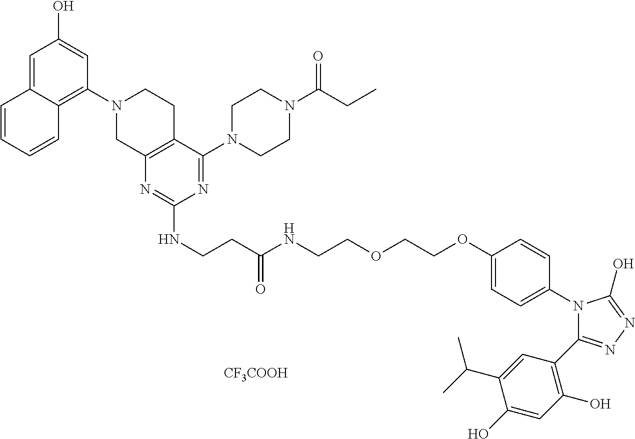 [SMILES: CCC(=O)N1CCN(c2nc(NCCC(=O)NCCOCCOc3ccc(-n4c(O)nnc4-c4cc(C(C)C)c(O)cc4O)cc3)nc3c2CCN(c2cc(O)cc4ccccc24)C3)CC1 ; "CF3COOH"]

### N-(2-(2-(4-(3-(2,4-dihydroxy-5-isopropylphenyl)-5-hydroxy-4H-1,2,4-triazol-4-yl)phenoxy)ethoxy)ethyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆) δ 11.87 (s, 1H), 9.77 (s, 1H), 9.60 (s, 1H), 9.39 (s, 1H), 8.10-7.95 (m, 2H), 7.68 (d, J=7.9 Hz, 1H), 7.45-7.36 (m, 1H), 7.32-7.24 (m, 1H), 7.09 (d, J=8.8 Hz, 2H), 6.98-6.85 (m, 3H), 6.81 (s, 1H), 6.75 (s, 1H), 6.25 (s, 1H), 4.10-4.08 (m, 4H), 3.76-3.75 (m, 5H), 3.58-3.56 (m, 6H), 3.46 (d, J=5.9 Hz, 3H), 3.24 (d, J=5.4 Hz, 4H), 2.96-2.95 (m, 3H), 2.46-2.29 (m, 5H), 1.00-0.99 (m, 9H). LCMS (ESI): m/z found 901.4 [M−CF3COOH+H]⁺.

### Compound 74

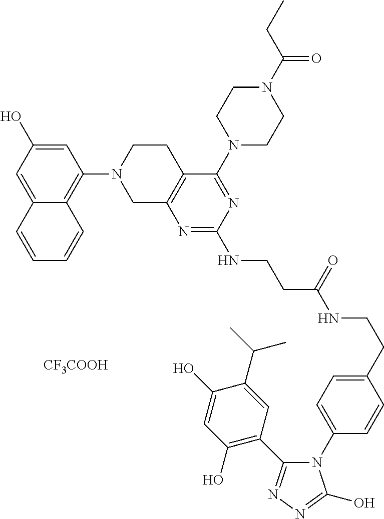 [SMILES: "CF3COOH" ; CCC(=O)N1CCN(c2nc(NCCC(=O)NCCc3ccc(-n4c(O)nnc4-c4cc(C(C)C)c(O)cc4O)cc3)nc3c2CCN(c2cc(O)cc4ccccc24)C3)CC1]

### N-(4-(3-(2,4-dihydroxy-5-isopropylphenyl)-5-hydroxy-4H-1,2,4-triazol-4-yl)phenethyl)-3-((7-(3-hydroxynaphthalen-1-yl)-4-(4-propionylpiperazin-1-yl)-5,6,7,8-tetrahydropyrido[3,4-d]pyrimidin-2-yl)amino)propanamide, trifluoroacetic acid

¹H NMR (400 MHz, DMSO-d₆) δ 11.90 (s, 1H), 9.74 (s, 1H), 9.58 (s, 1H), 9.37 (s, 1H), 8.10 (s, 1H), 7.99 (d, J=8.1 Hz, 1H), 7.68 (d, J=8.1 Hz, 1H), 7.43-7.37 (m, 1H), 7.30-7.25 (m, 1H), 7.21 (d, J=8.1 Hz, 2H), 7.11 (d, J=8.2 Hz, 2H), 6.89 (s, 1H), 6.78 (s, 1H), 6.75 (s, 1H), 6.25 (s, 1H), 4.15 (s, 2H), 3.83 (s, 4H), 3.59-3.58 (m, 7H), 3.26 (s, 5H), 2.99-2.89 (m, 3H), 2.72 (d, J=7.5 Hz, 3H), 2.42 (s, 2H), 2.38-2.32 (m, 2H), 1.07-0.90 (m, 9H). LCMS (ESI): m/z found 841.8 [M−CF3COOH+H]⁺.

### Compound 75

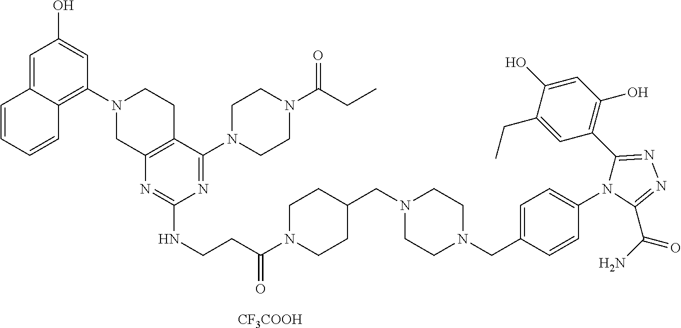 [SMILES: CCC(=O)N1CCN(c2nc(NCCC(=O)N3CCC(CN4CCN(Cc5ccc(-n6c(C(N)=O)nnc6-c6cc(CC)c(O)cc6O)cc5)CC4)CC3)nc3c2CCN(c2cc(O)cc4ccccc24)C3)CC1 ; "CF3COOH"]

### 5-(5-ethyl-2,4-dihydroxyphenyl)-4-(4-((4-((1-(3-((7-(3-hydroxynaphthalen-1-yl)-4-(4-propionylpiperazin-1-yl)-5,6,7,8-tetrahydropyrido[3,4-d]pyrimidin-2-yl)amino)propanoyl)piperidin-4-yl)methyl)piperazin-1-yl)methyl)phenyl)-4H-1,2,4-triazole-3-carboxamide, trifluoroacetic acid

¹H NMR (400 MHz, DMSO-d₆) δ 9.81-9.72 (m, 2H), 8.31 (s, 1H), 7.99 (d, J=8.6 Hz, 1H), 7.80-7.59 (m, 3H), 7.46-7.23 (m, 6H), 6.90 (s, 1H), 6.75 (s, 1H), 6.61 (s, 1H), 6.31 (s, 1H), 4.48-4.37 (m, 2H), 4.15 (s, 3H), 3.94-3.78 (m, 9H), 3.50-3.38 (m, 7H), 3.30-3.20 (m, 2H), 3.09-2.83 (m, 10H), 2.69-2.58 (m, 4H), 2.41-2.30 (m, 2H), 2.29-2.18 (m, 2H), 1.78-1.65 (m, 2H), 1.02 (t, J=7.4 Hz, 5H), 0.92-0.79 (m, 3H). LCMS (ESI): m/z found 1006.5[M−CF3COOH+H]⁺.

### Compound 76

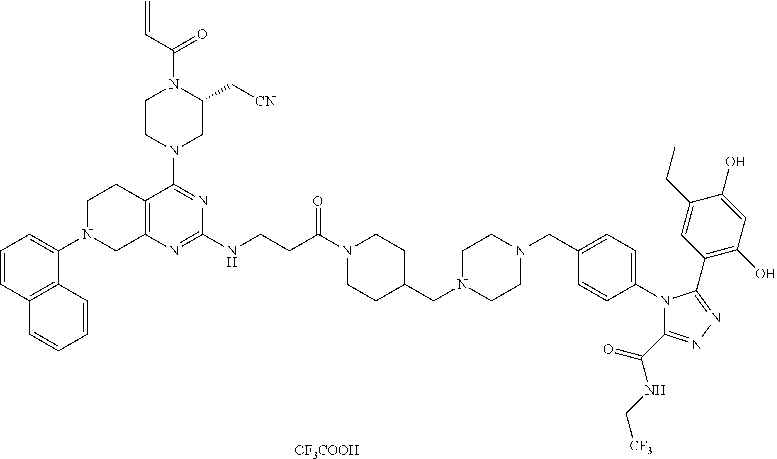 [SMILES: C=CC(=O)N1CCN(c2nc(NCCC(=O)N3CCC(CN4CCN(Cc5ccc(-n6c(C(=O)NCC(F)(F)F)nnc6-c6cc(CC)c(O)cc6O)cc5)CC4)CC3)nc3c2CCN(c2cccc4ccccc24)C3)C[C@H]1CC#N ; "CF3COOH"]

### (R)-4-(4-((4-((1-(3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(5-ethyl-2,4-dihydroxyphenyl)-N-(2,2,2-trifluoroethyl)-4H-1,2,4-triazole-3-carboxamide, trifluoroacetic acid

¹H NMR (400 MHz, DMSO-d₆) δ 9.74-9.63 (m, 2H), 8.15 (d, J=5.2 Hz, 1H), 7.96-7.93 (m, 1H), 7.83 (s, 1H), 7.69 (d, J=8.1 Hz, 1H), 7.59-7.17 (m, 10H), 6.95-6.87 (m, 1H), 6.67 (s, 1H), 6.31 (s, 1H), 6.21 (d, J=15.9 Hz, 1H), 5.80 (d, J=10.5 Hz, 1H), 4.95-4.79 (m, 1H), 4.45-4.32 (m, 3H), 4.21 (s, 3H), 3.97-4.80 (m, 3H), 3.66-3.48 (m, 5H), 3.31-3.18 (m, 3H), 3.08-2.80 (m, 9H), 2.68-2.53 (m, 3H), 2.29-2.23 (m, 3H), 2.03-1.88 (m, 2H), 1.78-1.64 (m, 3H), 1.29-1.15 (m, 3H), 1.11-0.93 (m, 2H), 0.88 (t, J=7.4 Hz, 4H). LCMS (ESI): m/z found 1110.1[M—CF3COOH+H]⁺.

### Compound 77

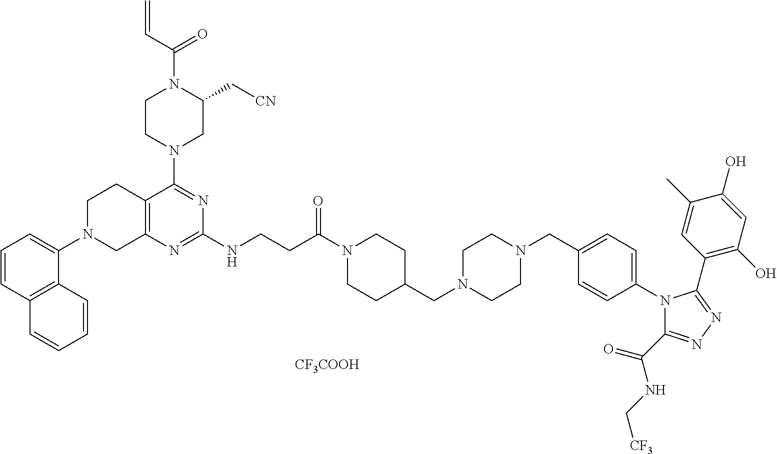 [SMILES: C=CC(=O)N1CCN(c2nc(NCCC(=O)N3CCC(CN4CCN(Cc5ccc(-n6c(C(=O)NCC(F)(F)F)nnc6-c6cc(C)c(O)cc6O)cc5)CC4)CC3)nc3c2CCN(c2cccc4ccccc24)C3)C[C@H]1CC#N ; "CF3COOH"]

### (R)-4-(4-((4-((1-(3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(2,4-dihydroxy-5-methylphenyl)-N-(2,2,2-trifluoroethyl)-4H-1,2,4-triazole-3-carboxamide, trifluoroacetic acid

¹H NMR (400 MHz, DMSO-d₆) δ 9.74-9.64 (m, 2H), 8.17 (d, J=4.8 Hz, 1H), 8.16-7.93 (m, 2H), 7.69 (d, J=8.0 Hz, 1H), 7.69-7.32 (m, 10H), 7.21 (d, J=7.2 Hz, 1H), 6.97-6.78 (m, 1H), 6.28 (s, 1H), 6.22 (d, J=17.6 Hz, 1H), 5.81 (d, J=11.6 Hz, 1H), 4.95-4.77 (m, 1H), 4.53-4.25 (m, 4H), 4.21 (s, 3H), 4.04-3.75 (m, 4H), 3.70-3.48 (m, 5H), 3.31-3.18 (m, 4H), 3.08-2.80 (m, 9H), 2.68-2.53 (m, 3H), 2.01-1.90 (m, 1H), 1.87 (s, 5H), 1.82-1.70 (m, 3H), 1.11-0.97 (m, 3H). LCMS (ESI): m/z found 1096.2[M−CF3COOH+H]⁺.

### Compound 78

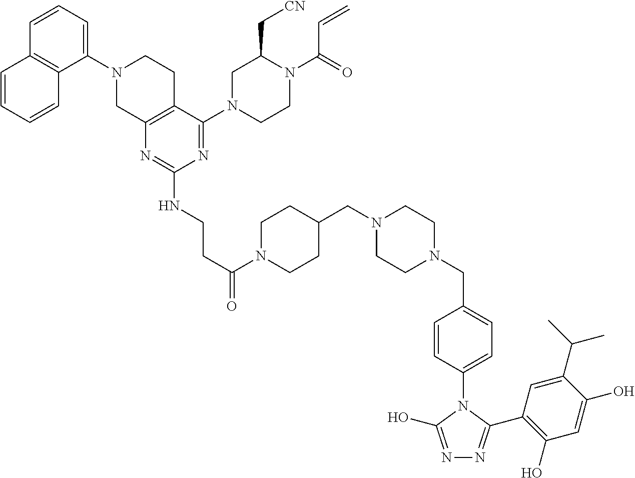 [SMILES: C=CC(=O)N1CCN(c2nc(NCCC(=O)N3CCC(CN4CCN(Cc5ccc(-n6c(O)nnc6-c6cc(C(C)C)c(O)cc6O)cc5)CC4)CC3)nc3c2CCN(c2cccc4ccccc24)C3)C[C@H]1CC#N]

### (R)-2-(1-acryloyl-4-(2-((3-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-3-oxopropyl)amino)-7-(naphthalen-1-yl)-5,6,7,8-tetrahydropyrido[3,4-d]pyrimidin-4-yl)piperazin-2-yl)acetonitrile

¹H NMR (400 MHz, DMSO-d₆): δ 11.96 (s, 1H), 9.70-9.35 (m, 2H), 8.17 (s, 1H), 8.00-7.80 (m, 2H), 7.75-7.13 (m, 10H), 6.94-6.78 (m, 2H), 6.30-6.14 (m, 2H), 5.80 (d, J=11.2 Hz, 1H), 5.00-4.78 (m, 1H), 4.52-4.36 (m, 3H), 4.25-4.18 (m, 2H), 4.12-3.85 (m, 7H), 3.35-3.18 (m, 5H), 3.08-2.88 (m, 9H), 2.69-2.62 (m, 3H), 2.59-2.55 (m, 2H), 2.06-1.88 (m, 2H), 1.79-1.68 (m, 2H), 1.32-1.22 (m, 2H), 1.10-0.90 (m, 8H). LCMS (ESI): m/z found 1014.2[M+H]⁺.

### Compound 79

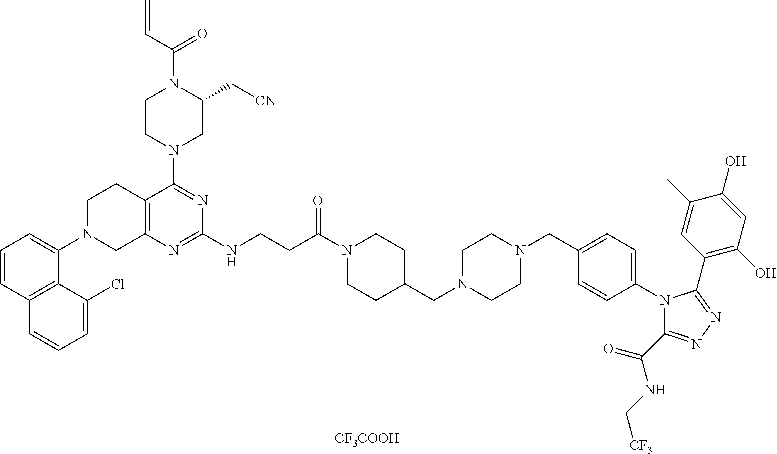 [SMILES: C=CC(=O)N1CCN(c2nc(NCCC(=O)N3CCC(CN4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@H]1CC#N ; "CF3COOH"]

### (R)-4-(4-((4-((1-(3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(2,4-dihydroxy-5-methylphenyl)-N-(2,2,2-trifluoroethyl)-4H-1,2,4-triazole-3-carboxamide, trifluoroacetic acid

¹H NMR (400 MHz, DMSO-d₆): δ 9.81-9.61 (m, 3H), 8.01-7.76 (m, 4H), 7.64-7.32 (m, 9H), 6.98-6.68 (m, 2H), 6.35-6.14 (m, 2H), 5.79 (d, J=10.0 Hz, 1H), 5.00-4.75 (m, 2H), 4.55-4.20 (m, 9H), 3.98-3.78 (m, 10H), 3.68-3.32 (m, 9H), 3.15-2.85 (m, 9H), 2.72-2.59 (m, 2H), 1.87 (s, 3H), 1.78-1.70 (m, 3H), 1.12-0.96 (m, 2H). LCMS (ESI): m/z found 1129.3[M−CF3COOH+H]⁺.

### Compound 80

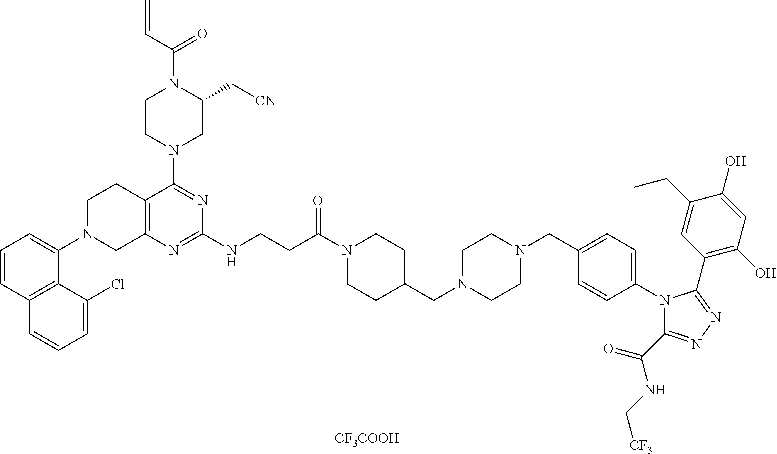 [SMILES: C=CC(=O)N1CCN(c2nc(NCCC(=O)N3CCC(CN4CCN(Cc5ccc(-n6c(C(=O)NCC(F)(F)F)nnc6-c6cc(CC)c(O)cc6O)cc5)CC4)CC3)nc3c2CCN(c2cccc4cccc(Cl)c24)C3)C[C@H]1CC#N ; "CF3COOH"]

### (R)-4-(4-((4-((1-(3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(5-ethyl-2,4-dihydroxyphenyl)-N-(2,2,2-trifluoroethyl)-4H-1,2,4-triazole-3-carboxamide, trifluoroacetic acid

¹H NMR (400 MHz, DMSO-d₆): δ 9.90-9.60 (m, 3H), 8.05-7.76 (m, 3H), 7.64-7.26 (m, 9H), 7.16-7.00 (m, 1H), 6.94-6.76 (m, 1H), 6.67 (s, 1H), 6.36-6.16 (m, 2H), 5.79 (d, J=10.8 Hz, 1H), 5.07-4.72 (m, 2H), 4.46-4.32 (m, 4H), 4.05-3.90 (m, 11H), 3.65-3.40 (m, 7H), 3.18-2.93 (m, 9H), 2.71-2.58 (m, 5H), 2.31-2.22 (m, 2H), 2.05-1.88 (m, 1H), 1.80-1.69 (m, 2H), 1.18-0.97 (m, 2H), 0.88 (t, J=7.6 Hz, 3H). LCMS (ESI): m/z found 1143.3[M−CF3COOH+H]⁺.

### Compound 81

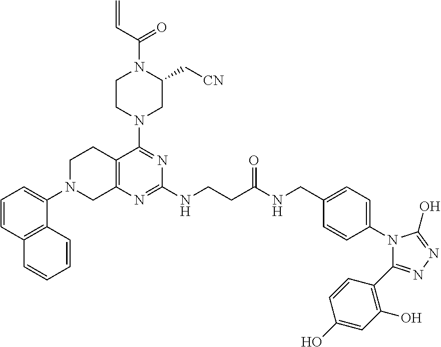 [SMILES: C=CC(=O)N1CCN(c2nc(NCCC(=O)NCc3ccc(-n4c(O)nnc4-c4ccc(O)cc4O)cc3)nc3c2CCN(c2cccc4ccccc24)C3)C[C@H]1CC#N]

### (R)-3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)-N-(4-(3-(2,4-dihydroxyphenyl)-5-hydroxy-4H-1,2,4-triazol-4-yl)benzyl)propenamide

¹H NMR (400 MHz, DMSO-d₆): δ 11.88 (s, 1H), 9.62 (d, J=26.0 Hz, 2H), 8.47 (s, 1H), 8.25-8.11 (m, 1H), 8.01-7.89 (m, 1H), 7.75-7.40 (m, 5H), 7.33-6.76 (m, 8H), 6.30-6.09 (m, 3H), 5.79 (d, J=10.4 Hz, 1H), 5.02-4.75 (m, 1H), 4.44-4.07 (m, 7H), 3.66-3.56 (m, 4H), 3.28-3.12 (m, 4H), 2.95 (d, J=18.7 Hz, 4H). LCMS (ESI): m/z found 806.8[M+H]⁺.

### Compound 82

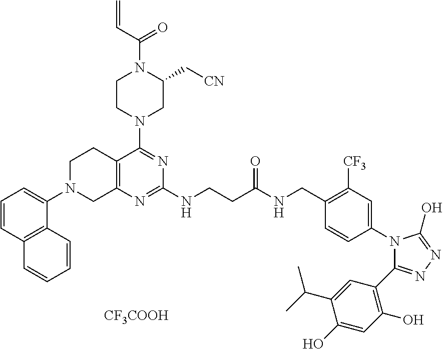 [SMILES: C=CC(=O)N1CCN(c2nc(NCCC(=O)NCc3ccc(-n4c(O)nnc4-c4cc(C(C)C)c(O)cc4O)cc3C(F)(F)F)nc3c2CCN(c2cccc4ccccc24)C3)C[C@H]1CC#N ; "CF3COOH"]

### (R)-3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)-2-(trifluoromethyl)benzyl)propanamide, trifluoroacetic acid

¹H NMR (400 MHz, DMSO-d₆): δ 12.01 (s, 1H), 9.59 (s, 1H), 9.35 (s, 1H), 8.58 (s, 1H), 8.21-8.12 (m, 1H), 7.98-7.91 (m, 1H), 7.71-7.39 (m, 8H), 7.20 (d, J=7.6 Hz, 1H), 7.04-6.77 (m, 2H), 6.20 (d, J=19.2 Hz, 2H), 5.79 (d, J=9.6 Hz, 1H), 5.02-4.74 (m, 1H), 4.48-4.39 (m, 3H), 4.20 (s, 2H), 3.60-3.57 (m, 3H), 3.38-3.14 (m, 5H), 3.06-2.88 (m, 5H), 2.60-2.54 (m, 3H), 1.03 (d, J=6.8 Hz, 6H). LCMS (ESI): m/z found 916.8[M−CF3COOH+H]⁺.

### Compound 83

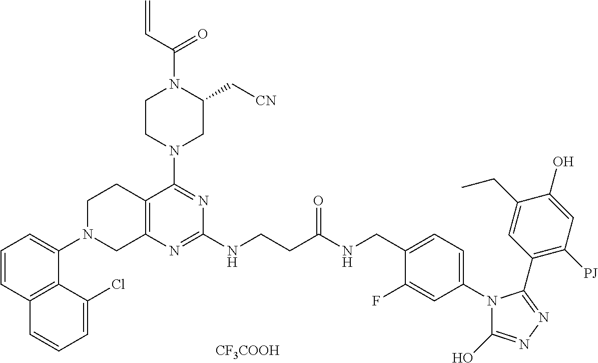

### (R)-3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)-N-(4-(3-(5-ethyl-2,4-dihydroxyphenyl)-5-hydroxy-4H-1,2,4-triazol-4-yl)-2-fluorobenzyl)propanamide, trifluoroacetic acid

¹H NMR (400 MHz, DMSO-d₆): δ 11.95 (d, J=0.4 Hz, 1H), 9.58 (s, 1H), 9.37 (s, 1H), 8.46 (s, 1H), 7.94 (d, J=7.6 Hz, 1H), 7.83-7.75 (m, 1H), 7.66-6.81 (m, 9H), 6.27-6.15 (m, 2H), 5.78 (d, J=11.0 Hz, 1H), 4.34-4.20 (m, 4H), 3.82-3.67 (m, 6H), 3.50-3.40 (m, 5H), 3.18-3.00 (m, 4H), 2.71-2.65 (m, 2H), 2.40-2.34 (m, 3H), 1.01 (t, J=7.2 Hz, 3H). LCMS (ESI): m/z found 886.8[M−CF3COOH+H]⁺.

### Compound 84

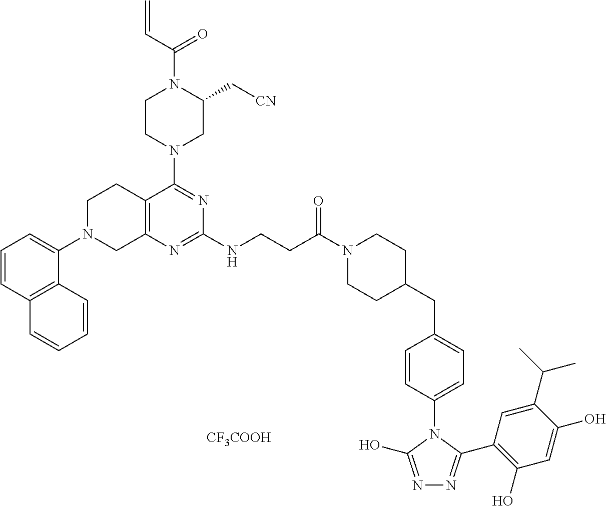 [SMILES: C=CC(=O)N1CCN(c2nc(NCCC(=O)N3CCC(Cc4ccc(-n5c(O)nnc5-c5cc(C(C)C)c(O)cc5O)cc4)CC3)nc3c2CCN(c2cccc4ccccc24)C3)C[C@H]1CC#N ; "CF3COOH"]

### (R)-2-(1-acryloyl-4-(2-((3-(4-(4-(3-(2,4-dihydroxy-5-isopropylphenyl)-5-hydroxy-4H-1,2,4-triazol-4-yl)benzyl)piperidin-1-yl)-3-oxopropyl)amino)-7-(naphthalen-1-yl)-5,6,7,8-tetrahydropyrido[3,4-d]pyrimidin-4-yl)piperazin-2-yl)acetonitrile, trifluoroacetic acid

¹H NMR (400 MHz, DMSO-d₆) δ 11.94 (s, 1H), 9.64 (s, 1H), 9.44 (s, 1H), 8.17 (s, 1H), 7.94 (s, 1H), 7.84-7.42 (m, 5H), 7.34-7.07 (m, 5H), 6.81 (m, 2H), 6.36-6.14 (m, 2H), 5.80 (d, J=10.3 Hz, 1H), 4.89 (m, 1H), 4.26 (m, 7H), 3.80 (d, J=12.7 Hz, 2H), 3.17 (s, 4H), 2.94 (m, 6H), 2.68 (m, 3H), 1.66 (m, 4H), 1.24 (s, 2H), 1.12-0.77 (m, 8H). LCMS (ESI): m/z found 916.1 [M−CF3COOH+H]⁺.

### Compound 85

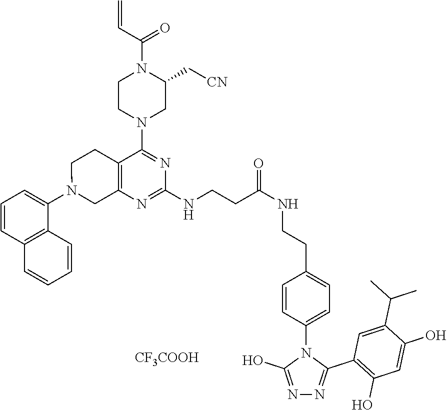 [SMILES: C=CC(=O)N1CCN(c2nc(NCCC(=O)NCCc3ccc(-n4c(O)nnc4-c4cc(C(C)C)c(O)cc4O)cc3)nc3c2CCN(c2cccc4ccccc24)C3)C[C@H]1CC#N ; "CF3COOH"]

### (R)-3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)phenethyl)propanamide, trifluoroacetic acid

¹H NMR (400 MHz, DMSO-d₆) δ 11.94 (s, 1H), 9.62 (s, 1H), 9.41 (s, 1H), 8.16 (s, 2H), 7.96 (s, 1H), 7.79-7.46 (m, 5H), 7.17 (m, 6H), 6.80 (s, 2H), 6.32-6.16 (m, 2H), 5.81 (d, J=11.0 Hz, 1H), 4.96 (s, 1H), 4.32 (m, 6H), 3.60 (s, 5H), 2.96 (m, 5H), 2.73 (d, J=7.5 Hz, 3H), 2.43 (s, 3H), 0.97 (d, J=6.8 Hz, 6H). LCMS (ESI): m/z found 862.0[M−CF3COOH+H]⁺.

### Compound 86

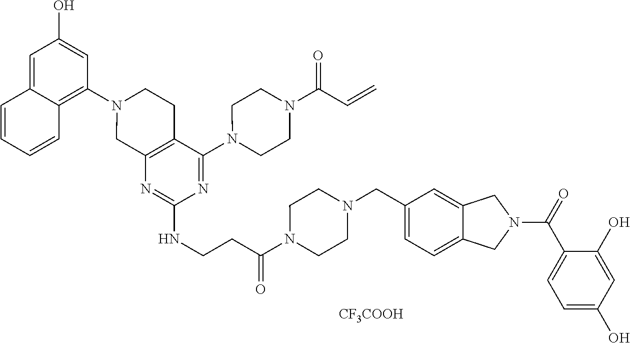 [SMILES: C=CC(=O)N1CCN(c2nc(NCCC(=O)N3CCN(Cc4ccc5c(c4)CN(C(=O)c4ccc(O)cc4O)C5)CC3)nc3c2CCN(c2cc(O)cc4ccccc24)C3)CC1 ; "CF3COOH"]

### 1-(4-(2-((3-(4-((2-(2,4-dihydroxybenzoyl)isoindolin-5-yl)methyl)piperazin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆) δ 10.38 (s, 1H), 10.11-9.92 (m, 1H), 9.75 (s, 1H), 7.99 (d, J=9.0 Hz, 1H), 7.68 (d, J=8.2 Hz, 1H), 7.60-7.05 (m, 6H), 6.99-6.68 (m, 3H), 6.40-6.10 (m, 3H), 5.75 (d, J=11.0 Hz, 1H), 4.79 (s, 4H), 4.35 (s, 3H), 4.20-3.57 (m, 12H), 3.35-3.17 (m, 5H), 2.91 (s, 5H), 2.67 (s, 4H). LCMS (ESI): m/z found 838.4 [M−CF3COOH+H]⁺.

### Compound 87

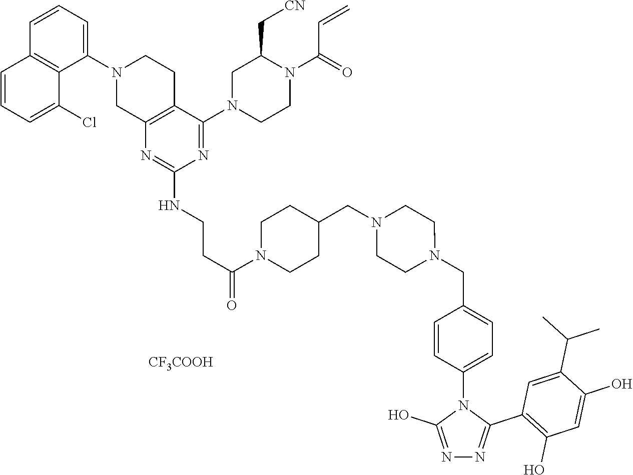 [SMILES: C=CC(=O)N1CCN(c2nc(NCCC(=O)N3CCC(CN4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@H]1CC#N ; "CF3COOH"]

### (R)-2-(1-acryloyl-4-(7-(8-chloronaphthalen-1-yl)-2-((3-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-3-oxopropyl)amino)-5,6,7,8-tetrahydropyrido[3,4-d]pyrimidin-4-yl)piperazin-2-yl)acetonitrile, trifluoroacetic acid

¹H NMR (400 MHz, DMSO-d₆) δ 11.95 (s, 1H), 9.65-9.48 (m, 2H), 8.06-7.72 (m, 3H), 7.49-7.37 (m, 9H), 7.21 (d, J=7.1 Hz, 2H), 6.82 (s, 2H), 6.34-6.15 (m, 2H), 5.80 (d, J=10.6 Hz, 1H), 4.92-4.73 (m, 2H), 4.31-4.45 (m, 3H), 4.29-4.09 (m, 4H), 3.83-3.75 (m, 5H), 3.18-3.11 (s, 3H), 3.05-2.95 (m, 6H), 2.70-2.60 (m, 5H), 2.42 (s, 2H), 2.01-1.88 (m, 2H), 1.75-1.68 (m, 3H), 1.24 (s, 2H), 1.13-1.01 (m, 1H), 0.97 (d, J=6.9 Hz, 7H). LCMS (ESI): m/z found 1048.5[M—CF3COOH+H]⁺.

### Compound 88

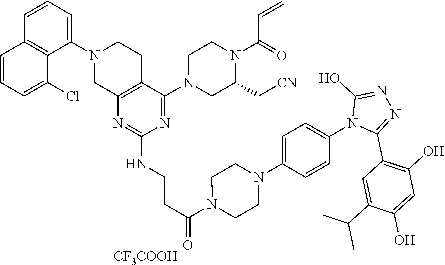 [SMILES: C=CC(=O)N1CCN(c2nc(NCCC(=O)N3CCN(c4ccc(-n5c(O)nnc5-c5cc(C(C)C)c(O)cc5O)cc4)CC3)nc3c2CCN(c2cccc4cccc(Cl)c24)C3)C[C@H]1CC#N ; "CF3COOH"]

### (R)-2-(1-acryloyl-4-(7-(8-chloronaphthalen-1-yl)-2-((3-(4-(4-(3-(2,4-dihydroxy-5-isopropylphenyl)-5-hydroxy-4H-1,2,4-triazol-4-yl)phenyl)piperazin-1-yl)-3-oxopropyl)amino)-5,6,7,8-tetrahydropyrido[3,4-d]pyrimidin-4-yl)piperazin-2-yl)acetonitrile, trifluoroacetic acid

¹H NMR (400 MHz, CD₃OD): δ 7.92-7.74 (m, 2H), 7.72-7.30 (m, 4H), 7.22-7.05 (m, 4H), 6.95-6.66 (m, 1H), 6.45-6.20 (m, 2H), 5.88 (s, 1H), 5.41 (s, 1H), 3.94-3.50 (m, 8H), 3.30-3.11 (m, 8H), 3.08-2.88 (m, 5H), 2.25-2.01 (m, 1H), 1.43-1.25 (m, 6H), 0.90 (d, J=6.4 Hz, 6H). LCMS (ESI): m/z found 937.4[M—CF3COOH+H]⁺.

### Compound 89

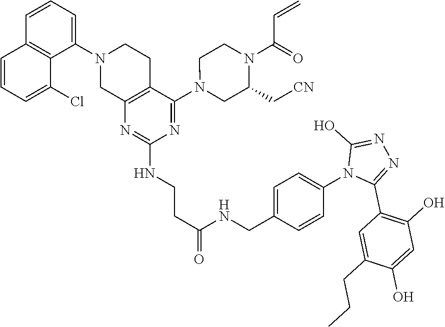 [SMILES: C=CC(=O)N1CCN(c2nc(NCCC(=O)NCc3ccc(-n4c(O)nnc4-c4cc(CCC)c(O)cc4O)cc3)nc3c2CCN(c2cccc4cccc(Cl)c24)C3)C[C@H]1CC#N]

### (R)-3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)-N-(4-(3-(2,4-dihydroxy-5-propylphenyl)-5-hydroxy-4H-1,2,4-triazol-4-yl)benzyl)propenamide

1H NMR (400 MHz, CD₃OD) δ 7.86-7.72 (m, 2H), 7.59-7.14 (m, 8H), 6.90-6.63 (m, 2H), 6.35-6.20 (m, 2H), 5.84 (d, J=9.4 Hz, 1H), 5.07-4.99 (m, 2H), 4.46-4.22 (m, 3H), 3.87-3.72 (m, 3H), 3.62-3.47 (m, 3H), 3.22-3.14 (m, 2H), 3.02-2.85 (m, 2H), 2.76-2.55 (m, 3H), 2.30 (t, J=6.8 Hz, 2H), 1.43-1.21 (m, 5H), 0.76 (t, J=6.0 Hz, 3H). LCMS (ESI): m/z found 882.7[M+H]⁺.

### Compound 90

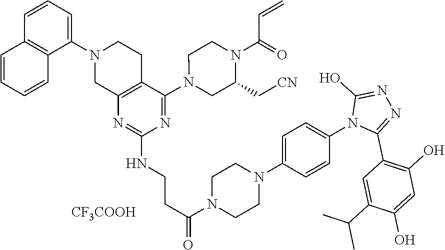 [SMILES: C=CC(=O)N1CCN(c2nc(NCCC(=O)N3CCN(c4ccc(-n5c(O)nnc5-c5cc(C(C)C)c(O)cc5O)cc4)CC3)nc3c2CCN(c2cccc4ccccc24)C3)C[C@H]1CC#N ; "CF3COOH"]

### (R)-2-(1-acryloyl-4-(2-((3-(4-(4-(3-(2,4-dihydroxy-5-isopropylphenyl)-5-hydroxy-4H-1,2,4-triazol-4-yl)phenyl)piperazin-1-yl)-3-oxopropyl)amino)-7-(naphthalen-1-yl)-5,6,7,8-tetrahydropyrido[3,4-d]pyrimidin-4-yl)piperazin-2-yl)acetonitrile, trifluoroacetic acid

¹H NMR (400 MHz, DMSO-d₆): δ 8.28-8.19 (m, 1H), 7.93-7.85 (m, 1H), 7.69-7.43 (m, 4H), 7.24-7.06 (m, 5H), 6.88-6.62 (m, 2H), 6.36-6.24 (m, 2H), 5.89-5.79 (m, 1H), 5.09-4.96 (m, 1H), 4.53-4.40 (m, 3H), 4.26-4.14 (m, 3H), 3.84-3.69 (m, 7H), 3.04-2.93 (m, 6H), 2.85-2.79 (m, 3H), 2.24-1.99 (m, 2H), 1.36-1.27 (m, 6H), 0.90-0.84 (m, 7H). LCMS (ESI): m/z found 904.0M−CF3COOH+H]⁺.

### Compound 91

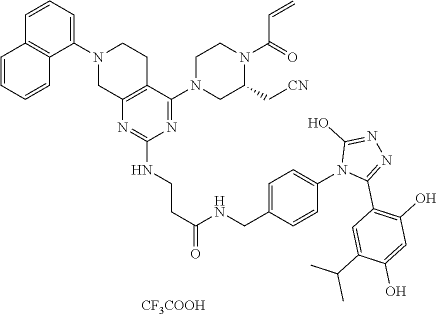 [SMILES: C=CC(=O)N1CCN(c2nc(NCCC(=O)NCc3ccc(-n4c(O)nnc4-c4cc(C(C)C)c(O)cc4O)cc3)nc3c2CCN(c2cccc4ccccc24)C3)C[C@H]1CC#N ; "CF3COOH"]

### (R)-3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)benzyl)propanamide, trifluoroacetic acid

¹H NMR (400 MHz, DMSO-d₆): δ 11.88 (s, 1H), 9.54-9.30 (m, 2H), 8.50 (s, 1H), 8.20-7.90 (m, 3H), 7.75-7.43 (m, 6H), 7.25-7.05 (m, 7H), 6.92-6.78 (m, 2H), 6.25-6.16 (m, 2H), 5.79 (d, J=10.4 Hz, 1H), 4.98-4.76 (m, 1H), 4.34-4.16 (m, 6H), 3.69-3.57 (m, 6H), 3.02-2.87 (m, 5H), 2.35-2.26 (m, 4H), 2.04-1.95 (m, 1H), 1.51-1.34 (m, 3H), 0.88-0.74 (m, 4H). LCMS (ESI): m/z found 848.9[M−CF3COOH+H]⁺.

### Compound 92

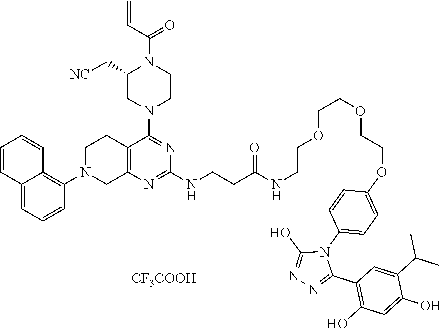 [SMILES: C=CC(=O)N1CCN(c2nc(NCCC(=O)NCCOCCOCCOc3ccc(-n4c(O)nnc4-c4cc(C(C)C)c(O)cc4O)cc3)nc3c2CCN(c2cccc4ccccc24)C3)C[C@@H]1CC#N ; "CF3COOH"]

### (S)-3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)-N-(2-(2-(2-(4-(3-(2,4-dihydroxy-5-isopropylphenyl)-5-hydroxy-4H-1,2,4-triazol-4-yl)phenoxy)ethoxy)ethoxy)ethyl)propanamide, trifluoroacetic acid

¹H NMR (400 MHz, DMSO-d₆): δ 11.91 (s, 1H), 9.72-9.40 (m, 2H), 8.27-7.91 (m, 3H), 7.82-7.47 (m, 4H), 7.30-6.79 (m, 7H), 6.38-6.12 (m, 2H), 5.81 (d, J=10.0 Hz, 1H), 5.02-4.80 (m, 1H), 4.53-4.39 (m, 1H), 4.20 (s, 2H), 4.07 (s, 2H), 3.78-3.72 (m, 5H), 3.61-3.57 (m, 6H), 3.28-3.22 (m, 8H), 3.03-2.94 (m, 4H), 2.81-2.77 (m, 1H), 2.48-2.40 (m, 3H), 0.98 (d, J=6.4 Hz, 6H). LCMS (ESI): m/z found 966.5[M−CF3COOH+H]⁺.

### Compound 93

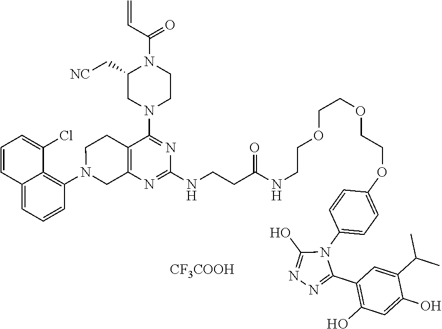 [SMILES: C=CC(=O)N1CCN(c2nc(NCCC(=O)NCCOCCOCCOc3ccc(-n4c(O)nnc4-c4cc(C(C)C)c(O)cc4O)cc3)nc3c2CCN(c2cccc4cccc(Cl)c24)C3)C[C@@H]1CC#N ; "CF3COOH"]

### (S)-3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)-N-(2-(2-(2-(4-(3-(2,4-dihydroxy-5-isopropylphenyl)-5-hydroxy-4H-1,2,4-triazol-4-yl)phenoxy)ethoxy)ethoxy)ethyl)propanamide, trifluoroacetic acid

¹H NMR (400 MHz, DMSO-d₆): δ 11.91 (s, 1H), 9.75-9.30 (m, 2H), 8.90-7.33 (m, 6H), 7.22-6.73 (m, 6H), 6.33-6.15 (m, 1H), 5.80 (d, J=9.2 Hz, 1H), 5.03-4.80 (m, 1H), 4.33-4.18 (m, 3H), 4.07 (s, 4H), 3.79-3.68 (m, 11H), 3.25-3.11 (m, 9H), 3.03-2.94 (m, 3H), 2.81-2.68 (m, 2H), 2.47-2.40 (m, 2H), 0.99 (d, J=6.0 Hz, 6H). LCMS (ESI): m/z found 1000.4[M−CF3COOH+H]⁺.

### Compound 94

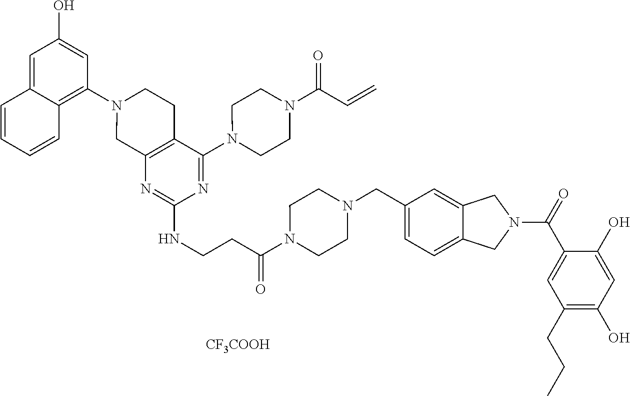 [SMILES: C=CC(=O)N1CCN(c2nc(NCCC(=O)N3CCN(Cc4ccc5c(c4)CN(C(=O)c4cc(CCC)c(O)cc4O)C5)CC3)nc3c2CCN(c2cc(O)cc4ccccc24)C3)CC1 ; "CF3COOH"]

### 1-(4-(2-((3-(4-((2-(2,4-dihydroxy-5-propylbenzoyl)isoindolin-5-yl)methyl)piperazin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 10.12 (s, 1H), 9.75-9.62 (m, 1H), 7.99 (d, J=8.7 Hz, 1H), 7.68 (d, J=7.5 Hz, 1H), 7.50-7.23 (m, 6H), 7.14-6.72 (m, 4H), 6.40 (s, 1H), 6.22-6.11 (m, 1H), 5.79-5.70 (m, 1H), 4.80 (s, 4H), 4.49-4.30 (m, 2H), 4.18-4.07 (m, 2H), 4.06-3.94 (m, 2H), 3.80-3.68 (m, 6H), 3.33-3.16 (m, 6H), 3.00-2.84 (m, 4H), 2.70-2.64 (m, 3H), 2.45-2.37 (m, 3H), 1.55-1.46 (m, 2H), 1.24 (s, 2H), 0.88 (t, J=7.4 Hz, 4H). LCMS (ESI): m/z found 880.3[M−CF3COOH+H]⁺.

### Compound 95

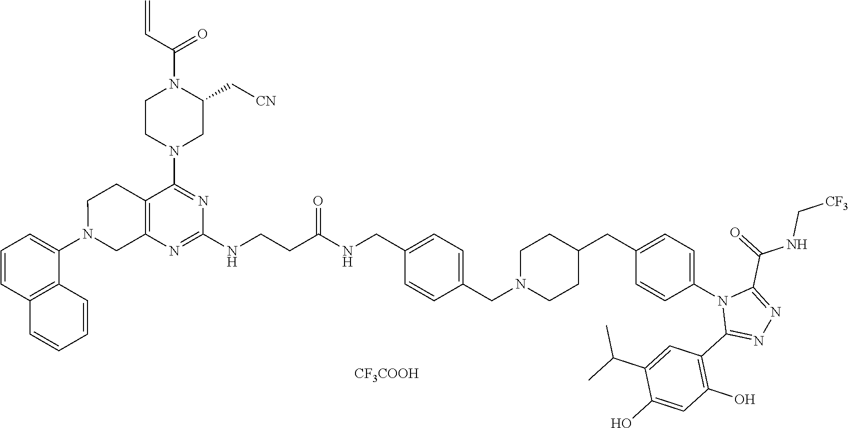 [SMILES: C=CC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cccc4ccccc24)C3)C[C@H]1CC#N ; "CF3COOH"]

### (R)-4-(4-((1-(4-((3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)propanamido)methyl)benzyl)piperidin-4-yl)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆): δ 9.79 (s, 1H), 9.62 (s, 1H), 9.44 (s, 1H), 8.54 (s, 1H), 8.17 (s, 1H), 7.95 (s, 1H), 7.72-7.66 (m, 1H), 7.62-7.53 (m, 2H), 7.58-7.41 (m, 3H), 7.39-7.18 (m, 6H), 7.10 (s, 1H), 6.97 (s, 1H), 6.92-6.76 (m, 1H), 6.60 (s, 1H), 6.34 (s, 1H), 6.21 (d, J=14.4 Hz, 1H), 5.84-5.75 (m, 1H), 5.04-4.77 (m, 2H), 4.39-4.29 (m, 3H), 4.28-4.15 (m, 4H), 4.02-3.89 (m, 4H), 3.39-3.29 (m, 4H), 3.24-3.10 (m, 3H), 3.08-2.66 (m, 10H), 1.82-1.63 (m, 3H), 1.48-1.32 (m, 2H), 1.28-1.15 (m, 2H), 0.82 (d, J=6.0 Hz, 6H). LCMS (ESI): m/z found 1144.6[M−CF3COOH+H]⁺.

### Compound 96

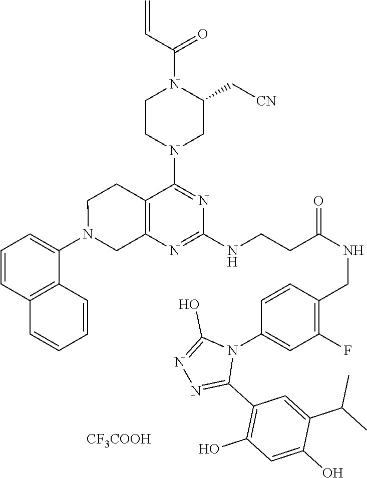 [SMILES: C=CC(=O)N1CCN(c2nc(NCCC(=O)NCc3ccc(-n4c(O)nnc4-c4cc(C(C)C)c(O)cc4O)cc3F)nc3c2CCN(c2cccc4ccccc24)C3)C[C@H]1CC#N ; "CF3COOH"]

### (R)-3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)-2-fluorobenzyl)propanamide, trifluoroacetic acid

¹H NMR (400 MHz, DMSO-d₆) δ 11.94 (s, 1H), 9.59 (s, 1H), 9.38 (s, 1H), 8.34 (s, 1H), 8.17 (s, 1H), 7.91 (s, 1H), 7.63 (d, J=8.3 Hz, 1H), 7.47 (m, 3H), 7.22 (m, 2H), 7.03 (d, J=10.8 Hz, 1H), 6.92 (d, J=7.4 Hz, 3H), 6.58 (s, 1H), 6.26-6.14 (m, 2H), 5.77 (d, J=11.3 Hz, 1H), 5.00 (s, 1H), 4.76 (s, 1H), 4.40 (s, 1H), 4.27 (d, J=4.9 Hz, 2H), 4.02 (s, 2H), 3.88 (s, 2H), 3.46 (d, J=6.8 Hz, 3H), 3.10-2.93 (m, 4H), 2.85 (s, 3H), 2.44 (m, 3H), 1.02 (d, J=6.9 Hz, 6H). LCMS (ESI): m/z found 866.8 [M−CF3COOH+H]⁺.

### Compound 97

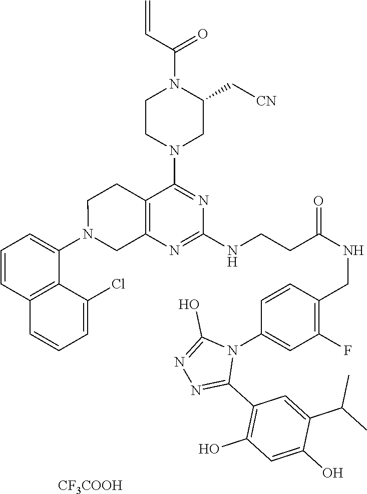 [SMILES: C=CC(=O)N1CCN(c2nc(NCCC(=O)NCc3ccc(-n4c(O)nnc4-c4cc(C(C)C)c(O)cc4O)cc3F)nc3c2CCN(c2cccc4cccc(Cl)c24)C3)C[C@H]1CC#N ; "CF3COOH"]

### (R)-3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)-N-(4-(3-(2,4-dihydroxy-5-isopropylphenyl)-5-hydroxy-4H-1,2,4-triazol-4-yl)-2-fluorobenzyl)propanamide, trifluoroacetic acid

¹H NMR (400 MHz, DMSO-d₆) δ 11.97 (s, 1H), 9.61 (s, 1H), 9.38 (s, 1H), 8.48 (s, 1H), 7.95 (d, J=8.0 Hz, 1H), 7.78 (s, 1H), 7.66-7.20 (m, 6H), 7.12-6.75 (m, 5H), 6.30-6.12 (m, 2H), 5.78 (d, J=10.6 Hz, 1H), 4.78 (s, 1H), 4.26 (m, 5H), 4.10-3.98 (m, 1H), 3.97-3.63 (m, 6H), 3.01 (m, 6H), 2.67 (s, 2H), 1.02 (d, J=6.7 Hz, 6H). LCMS (ESI): m/z found 900.8 [M−CF3COOH+H]⁺.

### Compound 98

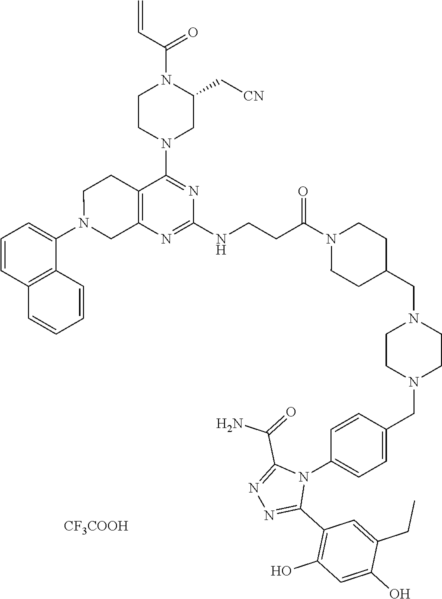 [SMILES: C=CC(=O)N1CCN(c2nc(NCCC(=O)N3CCC(CN4CCN(Cc5ccc(-n6c(C(N)=O)nnc6-c6cc(CC)c(O)cc6O)cc5)CC4)CC3)nc3c2CCN(c2cccc4ccccc24)C3)C[C@H]1CC#N ; "CF3COOH"]

### (R)-4-(4-((4-((1-(3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(5-ethyl-2,4-dihydroxyphenyl)-4H-1,2,4-triazole-3-carboxamide, trifluoroacetic acid

¹H NMR (400 MHz, DMSO-d₆) δ 9.72 (s, 1H), 8.32 (s, 1H), 8.17 (s, 1H), 7.95 (s, 1H), 7.76-7.68 (m, 3H), 7.57-7.55 (m, 2H), 7.49 (t, J=8.0 Hz, 1H), 7.37 (m, 4H), 7.21 (d, J=7.4 Hz, 1H), 6.61 (s, 1H), 6.38-6.15 (m, 2H), 5.81 (d, J=10.2 Hz, 1H), 4.88 (m, 2H), 4.41 (s, 3H), 4.21-4.20 (m, 4H), 4.05-4.03 (m, 2H), 3.82-3.81 (m, 2H), 3.62 (s, 6H), 3.32 (s, 2H), 3.17 (s, 3H), 3.00-2.99 (m, 6H), 2.65 (s, 3H), 2.24 (d, J=7.2 Hz, 3H), 1.99 (s, 1H), 1.73-1.72 (m, 2H), 1.24-1.23 (m, 2H), 1.00-1.09 (m, 2H), 0.86 (t, J=7.3 Hz, 4H). LCMS (ESI): m/z found 1028.0 [M−CF3COOH+H]⁺.

### Compound 99

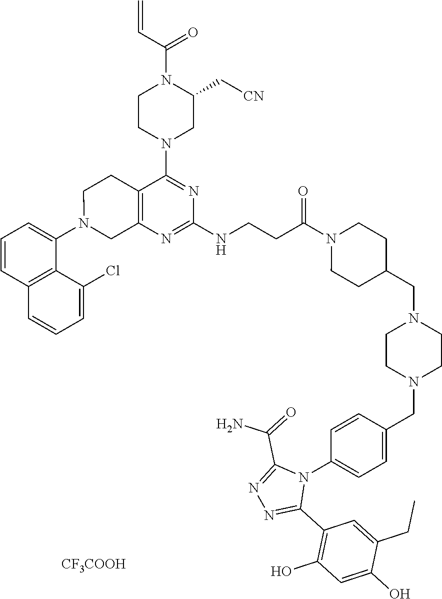 [SMILES: C=CC(=O)N1CCN(c2nc(NCCC(=O)N3CCC(CN4CCN(Cc5ccc(-n6c(C(N)=O)nnc6-c6cc(CC)c(O)cc6O)cc5)CC4)CC3)nc3c2CCN(c2cccc4cccc(Cl)c24)C3)C[C@H]1CC#N ; "CF3COOH"]

### (R)-4-(4-((4-((1-(3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)propanoyl)piperidin-4-yl)methyl)piperazin-1-yl)methyl)phenyl)-5-(5-ethyl-2,4-dihydroxyphenyl)-4H-1,2,4-triazole-3-carboxamide, trifluoroacetic acid

¹H NMR (400 MHz, DMSO-d₆) δ 9.73 (s, 1H), 8.32 (s, 1H), 7.98-7.31 (m, 11H), 6.94-6.58 (m, 1H), 6.35-6.16 (m, 1H), 5.80 (d, J=10.7 Hz, 1H), 5.12-3.90 (m, 12H), 3.93-2.87 (m, 16H), 2.72-2.59 (m, 2H), 2.30-2.20 (m, 3H), 1.97 (s, 2H), 1.81-1.60 (m, 3H), 1.34-0.95 (m, 6H), 0.86 (t, J=7.4 Hz, 5H). LCMS (ESI): m/z found 1061.5[M−CF3COOH+H]⁺.

### Compound 100

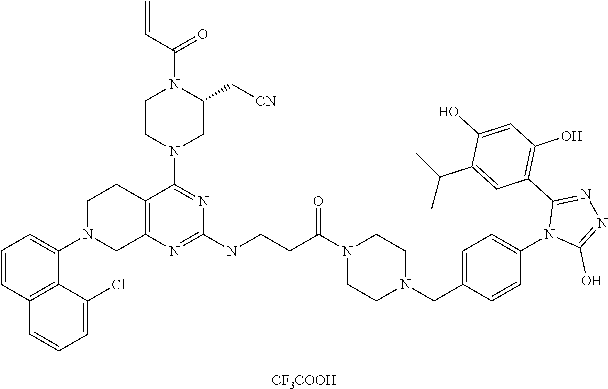

### (R)-2-(1-acryloyl-4-(7-(8-chloronaphthalen-1-yl)-2-((3-(4-(4-(3-(2,4-dihydroxy-5-isopropylphenyl)-5-hydroxy-4H-1,2,4-triazol-4-yl)benzyl)piperazin-1-yl)-3-oxopropyl)amino)-5,6,7,8-tetrahydropyrido[3,4-d]pyrimidin-4-yl)piperazin-2-yl)acetonitrile, trifluoroacetic acid

¹H NMR (400 MHz, DMSO-d₆) δ 11.98 (s, 1H), 10.47-9.11 (m, 3H), 7.97-7.65 (m, 2H), 7.64-7.05 (m, 8H), 6.89-6.75 (m, 2H), 6.29-6.15 (m, 2H), 5.85-5.72 (m, 1H), 4.56-2.73 (m, 28H), 1.00 (d, J=6.3 Hz, 9H). LCMS (ESI): m/z found 951.4[M−CF3COOH+H]⁺.

### Compound 101

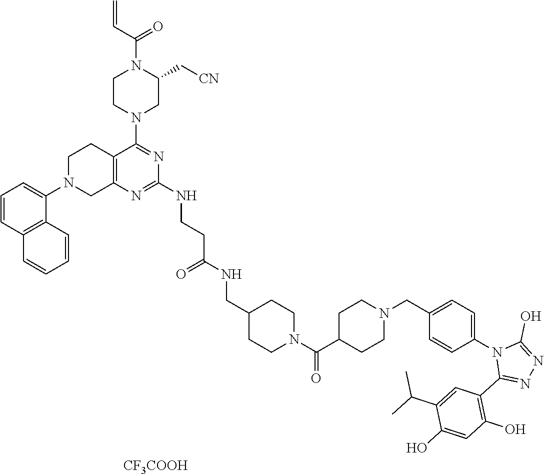 [SMILES: C=CC(=O)N1CCN(c2nc(NCCC(=O)NCC3CCN(C(=O)C4CCN(Cc5ccc(-n6c(O)nnc6-c6cc(C(C)C)c(O)cc6O)cc5)CC4)CC3)nc3c2CCN(c2cccc4ccccc24)C3)C[C@H]1CC#N ; "CF3COOH"]

### (R)-3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)-N-((1-(1-(4-(3-(2,4-dihydroxy-5-isopropylphenyl)-5-hydroxy-4H-1,2,4-triazol-4-yl)benzyl)piperidine-4-carbonyl)piperidin-4-yl)methyl)propanamide, trifluoroacetic acid

¹H NMR (400 MHz, DMSO-d₆) δ 11.98 (s, 1H), 9.69-9.25 (m, 3H), 8.25-7.88 (m, 3H), 7.69 (d, J=7.9 Hz, 1H), 7.59-7.44 (m, 5H), 7.31-7.18 (m, 3H), 6.87 (s, 2H), 6.30-6.15 (m, 2H), 5.80 (d, J=10.3 Hz, 1H), 5.01-4.76 (m, 1H), 4.30-4.15 (m, 6H), 4.01-3.92 (m, 3H), 3.63-3.55 (s, 5H), 3.38-3.21 (m, 6H), 3.02-2.91 (m, 9H), 2.45-2.37 (m, 4H), 1.82-1.57 (m, 7H), 1.07-0.80 (m, 8H). LCMS (ESI): m/z found 1057.1[M−CF3COOH+H]⁺.

### Compound 102

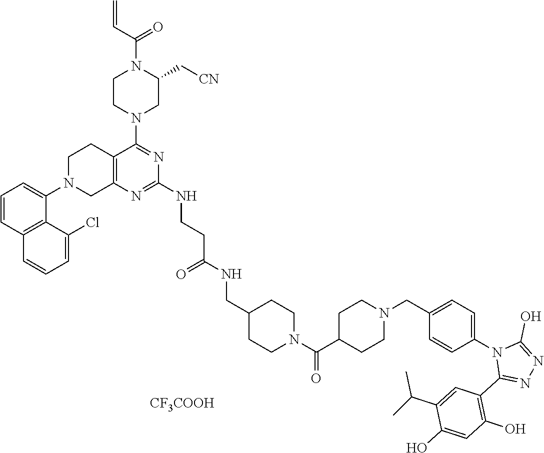 [SMILES: C=CC(=O)N1CCN(c2nc(NCCC(=O)NCC3CCN(C(=O)C4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@H]1CC#N ; "CF3COOH"]

### (R)-3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)-N-((1-(1-(4-(3-(2,4-dihydroxy-5-isopropylphenyl)-5-hydroxy-4H-1,2,4-triazol-4-yl)benzyl)piperidine-4-carbonyl)piperidin-4-yl)methyl)propanamide, trifluoroacetic acid

¹H NMR (400 MHz, DMSO-d₆): δ 11.98 (s, 1H), 9.69-9.27 (m, 3H), 8.03-7.91 (m, 2H), 7.82-7.76 (m, 1H), 7.647.23 (m, 9H), 6.92-6.78 (m, 2H), 6.30-6.15 (m, 2H), 5.79 (d, J=10.4 Hz, 1H), 4.38-4.16 (m, 6H), 3.95-3.76 (m, 6H), 3.62-3.44 (m, 7H), 3.15-2.90 (m, 10H), 2.70-2.63 (m, 1H), 2.35-2.30 (m, 2H), 1.82-1.29 (m, 8H), 1.05-0.85 (m, 10H). LCMS (ESI): m/z found 1090.8[M−CF3COOH+H]⁺.

### Compound 103

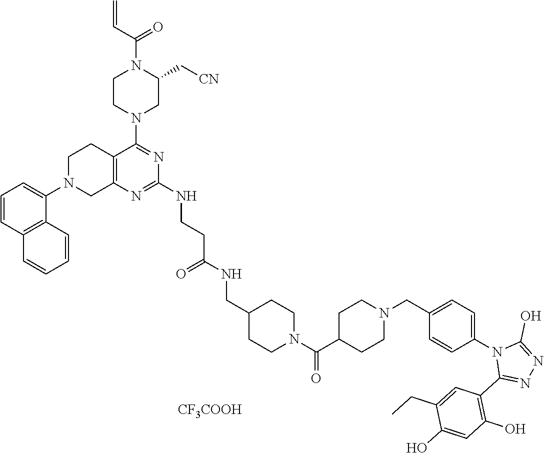 [SMILES: C=CC(=O)N1CCN(c2nc(NCCC(=O)NCC3CCN(C(=O)C4CCN(Cc5ccc(-n6c(O)nnc6-c6cc(CC)c(O)cc6O)cc5)CC4)CC3)nc3c2CCN(c2cccc4ccccc24)C3)C[C@H]1CC#N ; "CF3COOH"]

### (R)-3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)-N-((1-(1-(4-(3-(5-ethyl-2,4-dihydroxyphenyl)-5-hydroxy-4H-1,2,4-triazol-4-yl)benzyl)piperidine-4-carbonyl)piperidin-4-yl)methyl)propanamide, trifluoroacetic acid

¹H NMR (400 MHz, DMSO-d₆): δ 11.96 (s, 1H), 9.67-9.26 (m, 3H), 8.19-8.14 (m, 1H), 8.05-7.90 (m, 2H), 7.71-7.44 (m, 7H), 7.29-7.19 (m, 3H), 6.96-6.80 (m, 2H), 6.27-6.17 (m, 2H), 5.80 (d, J=12.4 Hz, 1H), 5.00-4.75 (m, 1H), 4.46-4.16 (m, 8H), 4.08-3.91 (m, 3H), 3.74-3.66 (m, 3H), 3.39-3.25 (m, 5H), 3.07-2.83 (m, 11H), 2.44-2.33 (m, 4H), 1.89-1.58 (m, 7H), 1.08-0.86 (m, 5H). LCMS (ESI): m/z found 1042.5[M−CF3COOH+H]⁺.

### Compound 104

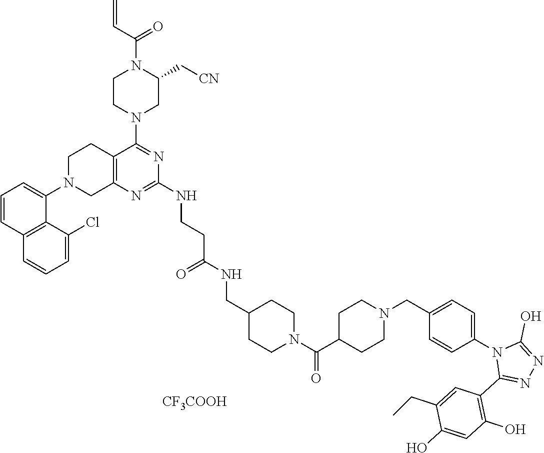 [SMILES: C=CC(=O)N1CCN(c2nc(NCCC(=O)NCC3CCN(C(=O)C4CCN(Cc5ccc(-n6c(O)nnc6-c6cc(CC)c(O)cc6O)cc5)CC4)CC3)nc3c2CCN(c2cccc4cccc(Cl)c24)C3)C[C@H]1CC#N ; "CF3COOH"]

### (R)-3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)-N-((1-(1-(4-(3-(5-ethyl-2,4-dihydroxyphenyl)-5-hydroxy-4H-1,2,4-triazol-4-yl)benzyl)piperidine-4-carbonyl)piperidin-4-yl)methyl)propanamide, trifluoroacetic acid

¹H NMR (400 MHz, DMSO-d₆): δ 11.96 (s, 1H), 9.66-9.29 (m, 3H), 7.97-7.91 (m, 2H), 7.80-7.75 (m, 1H), 7.66-7.21 (m, 9H), 6.95-6.75 (m, 2H), 6.29-6.13 (m, 2H), 5.79 (d, J=11.2 Hz, 1H), 4.41-4.16 (m, 6H), 4.10-3.88 (m, 3H), 3.57-3.51 (m, 4H), 3.16-2.84 (m, 13H), 2.70-2.63 (m, 2H), 2.42-2.32 (m, 5H), 1.90-1.54 (m, 8H), 1.09-0.80 (m, 6H). LCMS (ESI): m/z found 1076.5[M—CF3COOH+H]⁺.

### Compound 105

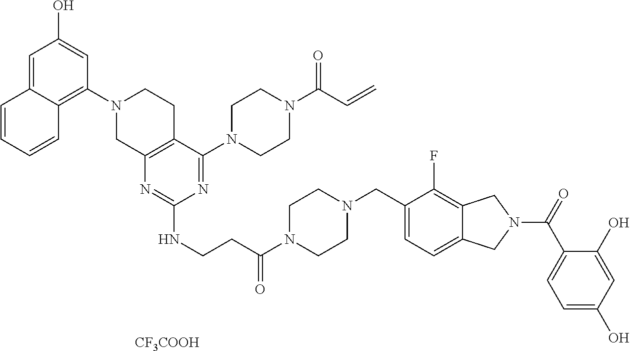 [SMILES: C=CC(=O)N1CCN(c2nc(NCCC(=O)N3CCN(Cc4ccc5c(c4F)CN(C(=O)c4ccc(O)cc4O)C5)CC3)nc3c2CCN(c2cc(O)cc4ccccc24)C3)CC1 ; "CF3COOH"]

### 1-(4-(2-((3-(4-((2-(2,4-dihydroxybenzoyl)-4-fluoroisoindolin-5-yl)methyl)piperazin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 10.34 (s, 1H), 9.77 (s, 2H), 7.99 (d, J=8.2 Hz, 1H), 7.69 (d, J=8.6 Hz, 1H), 7.44-7.14 (m, 5H), 6.94-6.70 (m, 3H), 6.43-6.11 (m, 3H), 5.75 (d, J=10.6 Hz, 1H), 4.85 (s, 4H), 4.38-4.11 (m, 4H), 3.92-3.68 (m, 10H), 3.65-3.54 (m, 6H), 2.98-2.63 (m, 9H). LCMS (ESI): m/z found 856.8[M−CF3COOH+H]⁺.

### Compound 106

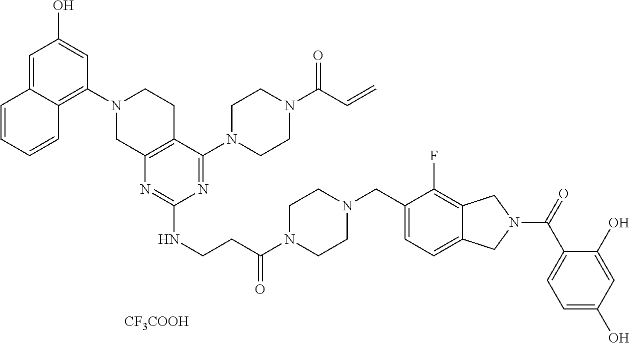 [SMILES: C=CC(=O)N1CCN(c2nc(NCCC(=O)N3CCN(Cc4ccc5c(c4F)CN(C(=O)c4ccc(O)cc4O)C5)CC3)nc3c2CCN(c2cc(O)cc4ccccc24)C3)CC1 ; "CF3COOH"]

### 1-(4-(2-((3-(4-((2-(2,4-dihydroxybenzoyl)-4-fluoroisoindolin-5-yl)methyl)piperazin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 10.34 (s, 1H), 9.77 (s, 2H), 7.99 (d, J=8.2 Hz, 1H), 7.69 (d, J=8.6 Hz, 1H), 7.44-7.14 (m, 5H), 6.94-6.70 (m, 3H), 6.43-6.11 (m, 3H), 5.75 (d, J=10.6 Hz, 1H), 4.85 (s, 4H), 4.38-4.11 (m, 4H), 3.92-3.68 (m, 10H), 3.65-3.54 (m, 6H), 2.98-2.63 (m, 9H). LCMS (ESI): m/z found 856.8[M−CF3COOH+H]⁺.

### Compound 107

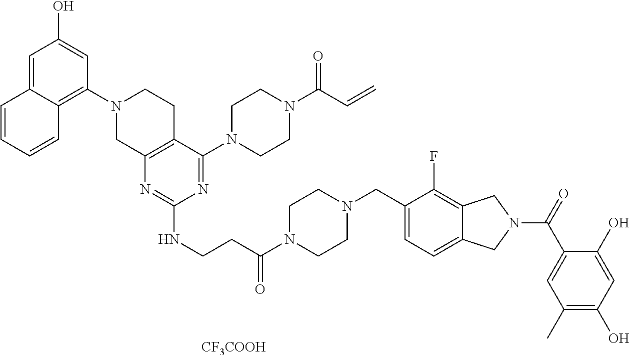 [SMILES: C=CC(=O)N1CCN(c2nc(NCCC(=O)N3CCN(Cc4ccc5c(c4F)CN(C(=O)c4cc(C)c(O)cc4O)C5)CC3)nc3c2CCN(c2cc(O)cc4ccccc24)C3)CC1 ; "CF3COOH"]

### 1-(4-(2-((3-(4-((2-(2,4-dihydroxy-5-methylbenzoyl)-4-fluoroisoindolin-5-yl)methyl)piperazin-1-yl)-3-oxopropyl)amino)-7-(3-hydroxynaphthalen-1-yl)-5,6,7,8-tetrahydropyrido[3,4-d]pyrimidin-4-yl)piperazin-1-yl)prop-2-en-1-one, trifluoroacetic acid

¹H NMR (400 MHz, DMSO-d₆): δ 10.05 (s, 1H), 9.72 (d, J=27.4 Hz, 2H), 7.99 (d, J=8.6 Hz, 1H), 7.68 (d, J=8.0 Hz, 1H), 7.45-7.16 (m, 5H), 7.07-6.71 (m, 5H), 6.41 (s, 1H), 6.17 (d, J=14.8 Hz, 1H), 5.74 (d, J=12.0 Hz, 1H), 4.84 (s, 4H), 4.50-4.25 (m, 2H), 4.13 (s, 2H), 3.84-3.67 (m, 10H), 3.29-3.21 (m, 4H), 3.00-2.83 (m, 6H), 2.70-2.64 (m, 3H), 2.03 (s, 3H). LCMS (ESI): m/z found 870.3[M−CF3COOH+H]⁺.

### Compound 108

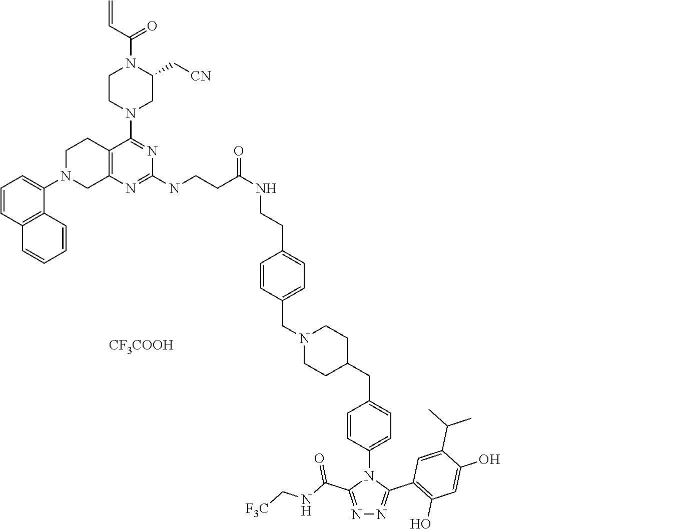

### (R)-4-(4-((1-(4-(2-(3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)propanamido)ethyl)benzyl)piperidin-4-yl)methyl)phenyl)-5-(2,4-dihydroxy-5-isopropylphenyl)-N-(2,2,2-trifluoroethyl)-4H-1,2,4-triazole-3-carboxamide, trifluoroacetic acid, trifluoroacetic acid

¹H NMR (400 MHz, DMSO-d₆) δ 9.79 (s, 1H), 9.61 (t, J=6.6 Hz, 1H), 9.40 (s, 1H), 8.20-8.08 (m, 2H), 7.97-7.91 (m, 1H), 7.69 (d, J=8.2 Hz, 1H), 7.55-7.54 (m, 2H), 7.52-7.46 (m, 1H), 7.41 (d, J=7.9 Hz, 2H), 7.26 (m, 7H), 6.60 (s, 1H), 6.34 (s, 1H), 6.21 (m, 1H), 5.80 (d, J=12.2 Hz, 1H), 4.89 (d, J=48.9 Hz, 2H), 4.37 (s, 3H), 4.21 (d, J=11.1 Hz, 4H), 3.98-3.93 (m, 2H), 3.58 (s, 2H), 3.31 (s, 4H), 3.21-3.12 (m, 2H), 2.92 (m, 7H), 2.78-2.72 (m, 2H), 2.54 (s, 2H), 2.43 (d, J=6.5 Hz, 2H), 1.76 (d, J=10.7 Hz, 4H), 1.40 (d, J=5.8 Hz, 2H), 0.82 (d, J=6.9 Hz, 6H). LCMS (ESI): m/z found 1158.6 [M−CF3COOH+H]⁺.

### Compound 109

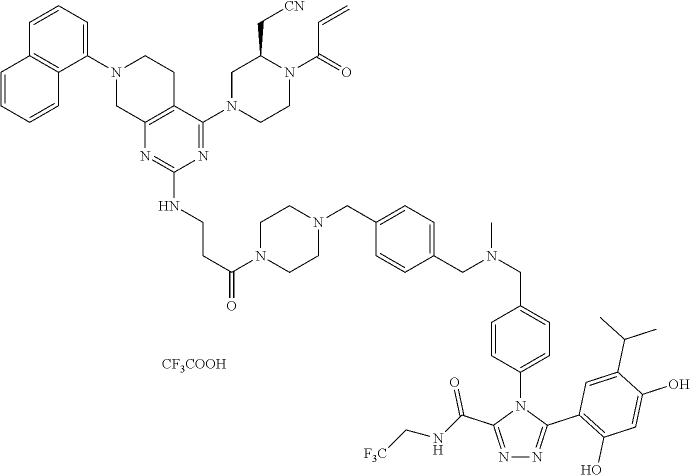 [SMILES: C=CC(=O)N1CCN(c2nc(NCCC(=O)N3CCN(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cccc4ccccc24)C3)C[C@H]1CC#N ; "CF3COOH"]

### (R)-4-(4-(((4-((4-(3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)propanoyl)piperazin-1-yl)methyl)benzyl)(methyl)amino)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆) δ 10.17 (s, 1H), 9.70 (d, J=27.9 Hz, 2H), 8.15 (s, 1H), 7.99-7.89 (m, 1H), 7.77-7.37 (m, 9H), 7.21 (d, J=7.4 Hz, 1H), 6.75 (s, 2H), 6.35-6.14 (m, 2H), 5.80 (d, J=11.8 Hz, 1H), 4.95 (s, 2H), 4.43 (s, 3H), 4.19 (s, 6H), 3.94 (dd, J=16.1, 9.0 Hz, 4H), 3.61 (s, 5H), 3.28 (d, J=50.5 Hz, 6H), 2.96 (d, J=20.5 Hz, 6H), 2.68 (s, 3H), 2.00 (d, J=7.6 Hz, 1H), 1.58 (s, 2H), 0.87 (dd, J=20.6, 6.3 Hz, 5H). LCMS (ESI): m/z found 1160.1 [M—CF3COOH+H]⁺.

### Compound 110

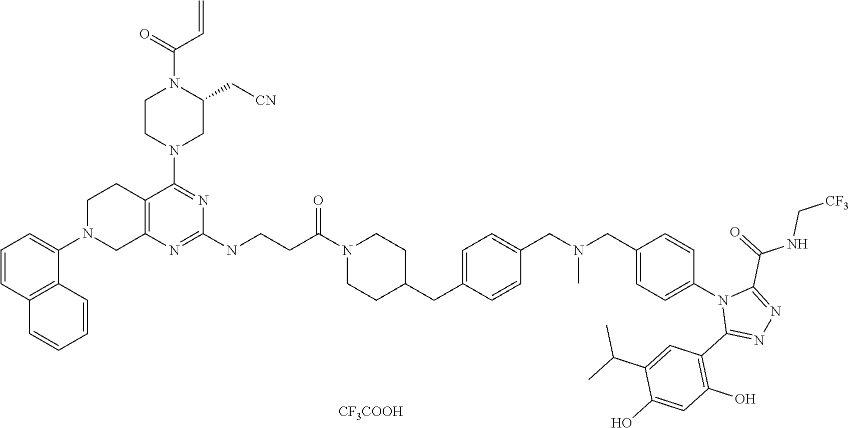

### (R)-4-(4-(((4-((1-(3-((4-(4-acryloyl-3-(cyanomethyl)piperazin-1-yl)-7-(naphthalen-1-yl)-5,6,7,8-tetrahydropyrido[3,4-d]pyrimidin-2-yl)amino)propanoyl)piperidin-4-yl)methyl)benzyl)(methyl)amino)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆) δ 9.88 (s, 1H), 9.69 (d, J=8.5 Hz, 2H), 8.17 (s, 1H), 7.96 (s, 2H), 7.69 (d, J=7.6 Hz, 2H), 7.60-7.18 (m, 12H), 6.76 (s, 2H), 6.29 (s, 1H), 6.20 (d, J=17.0 Hz, 1H), 5.80 (d, J=10.5 Hz, 1H), 4.90-4.81 (m, 1H), 4.40 (s, 6H), 4.21 (s, 6H), 3.96 (s, 3H), 3.83 (s, 1H), 3.59 (s, 4H), 3.21 (s, 3H), 2.95 (d, J=16.9 Hz, 5H), 2.63 (s, 3H), 1.59 (s, 1H), 1.13 (s, 2H), 0.90 (d, J=6.2 Hz, 6H). LCMS (ESI): m/z found 1158.3 [M−CF3COOH+H]⁺.

### Compound 111

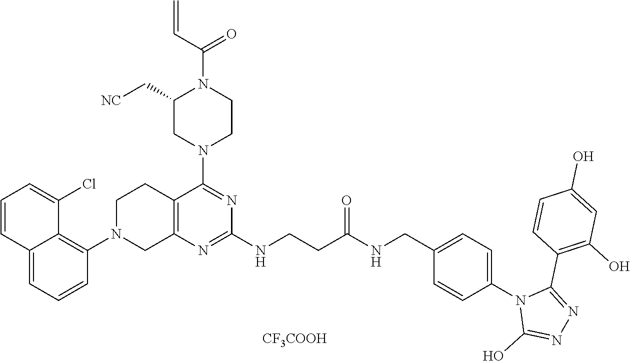 [SMILES: C=CC(=O)N1CCN(c2nc(NCCC(=O)NCc3ccc(-n4c(O)nnc4-c4ccc(O)cc4O)cc3)nc3c2CCN(c2cccc4cccc(Cl)c24)C3)C[C@@H]1CC#N ; "CF3COOH"]

### (S)-3-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)amino)-N-(4-(3-(2,4-dihydroxyphenyl)-5-hydroxy-4H-1,2,4-triazol-4-yl)benzyl)propanamide, trifluoroacetic acid

¹H NMR (400 MHz, DMSO-d₆) δ 11.89 (s, 1H), 9.75-9.50 (m, 2H), 8.59-8.38 (m, 1H), 8.03-7.67 (m, 3H), 7.65-7.31 (m, 4H), 7.26-7.00 (m, 5H), 6.95-6.75 (m, 1H), 6.28-6.12 (m, 3H), 5.79 (d, J=10.3 Hz, 1H), 5.08-4.70 (m, 2H), 4.46-4.18 (m, 6H), 3.88-3.56 (m, 8H), 3.18-2.88 (m, 5H), 2.71-2.64 (m, 1H). LCMS (ESI): m/z found 840.3[M−CF3COOH+H]⁺.

### Compound 112

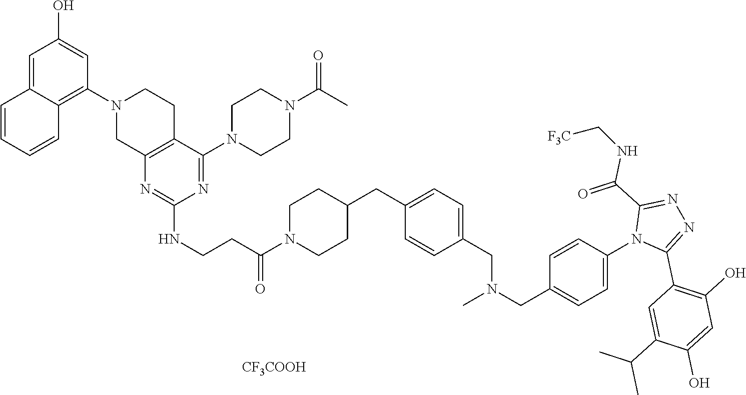 [SMILES: CC(=O)N1CCN(c2nc(NCCC(=O)N3CCC(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cc(O)cc4ccccc24)C3)CC1 ; "CF3COOH"]

### 4-(4-(((4-((1-(3-((4-(4-acetylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)benzyl)(methyl)amino)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆) δ 9.82-9.58 (m, 3H), 7.99 (d, J=8.4 Hz, 1H), 7.69 (d, J=7.8 Hz, 2H), 7.62-7.06 (m, 12H), 6.90 (s, 1H), 6.76 (s, 2H), 6.30 (s, 1H), 4.51-4.31 (m, 4H), 4.26-4.12 (m, 5H), 4.01-3.78 (m, 6H), 3.62 (s, 6H), 3.30-3.20 (m, 2H), 3.08-2.85 (m, 4H), 2.70-2.62 (m, 2H), 2.58-2.53 (m, 4H), 2.05 (s, 4H), 1.84-1.74 (m, 1H), 1.65-1.52 (m, 2H), 1.27-0.97 (m, 2H), 0.90 (d, J=6.6 Hz, 6H). LCMS (ESI): m/z found 1123.3[M−CF3COOH+H]⁺.

### Compound 113

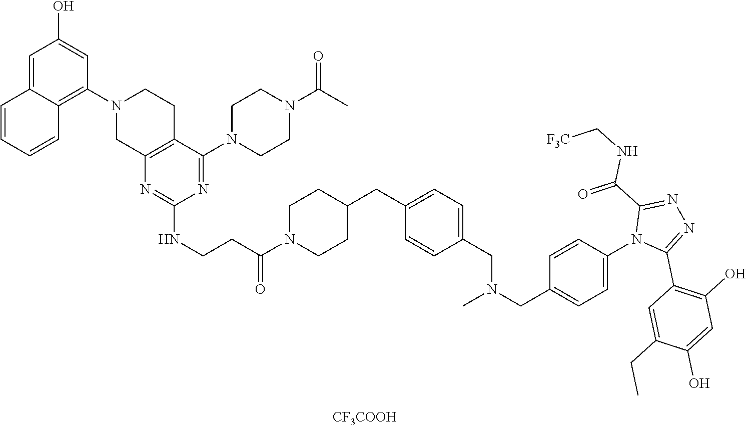 [SMILES: CCc1cc(-c2nnc(C(=O)NCC(F)(F)F)n2-c2ccc(CN(C)Cc3ccc(CC4CCN(C(=O)CCNc5nc6c(c(N7CCN(C(C)=O)CC7)n5)CCN(c5cc(O)cc7ccccc57)C6)CC4)cc3)cc2)c(O)cc1O ; "CF3COOH"]

### 4-(4-(((4-((1-(3-((4-(4-acetylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)benzyl)(methyl)amino)methyl)phenyl)-5-(5-ethyl-2,4-dihydroxyphenyl)-N-(2,2,2-trifluoroethyl)-4H-1,2,4-triazole-3-carboxamide, trifluoroacetic acid

¹H NMR (400 MHz, DMSO-d₆) δ 9.77-9.62 (m, 3H), 7.99 (d, J=9.2 Hz, 1H), 7.69 (d, J=8.1 Hz, 1H), 7.59-7.24 (m, 11H), 6.89 (s, 1H), 6.75 (s, 2H), 6.28 (s, 1H), 4.50-4.33 (m, 3H), 4.25-4.12 (m, 4H), 4.03-3.92 (m, 4H), 3.89-3.78 (m, 6H), 3.67-3.57 (m, 9H), 3.30-3.21 (m, 2H), 3.02-2.86 (m, 3H), 2.70-2.62 (m, 3H), 2.36-2.23 (m, 3H), 2.05 (s, 3H), 1.84-1.73 (m, 1H), 1.65-1.52 (m, 2H), 1.24 (s, 2H), 0.91 (t, J=7.2 Hz, 3H). LCMS (ESI): m/z found 1109.3[M−CF3COOH+H]⁺.

### Compound 114

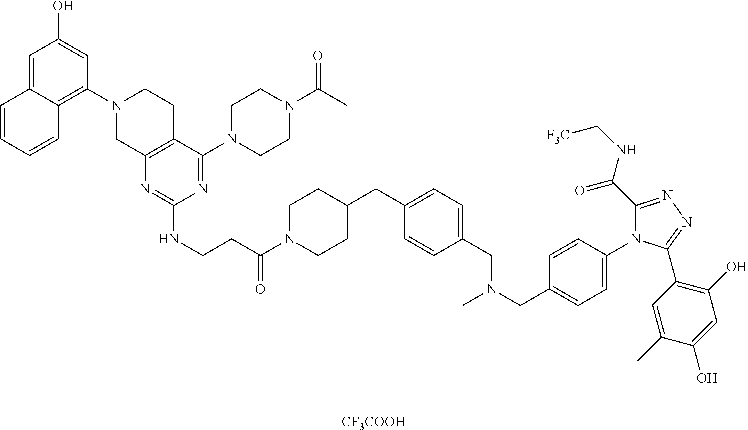 [SMILES: CC(=O)N1CCN(c2nc(NCCC(=O)N3CCC(Cc4ccc(CN(C)Cc5ccc(-n6c(C(=O)NCC(F)(F)F)nnc6-c6cc(C)c(O)cc6O)cc5)cc4)CC3)nc3c2CCN(c2cc(O)cc4ccccc24)C3)CC1 ; "CF3COOH"]

### 4-(4-(((4-((1-(3-((4-(4-acetylpiperazin-1-yl)-7-(3-hydroxynaphthalen-1-yl)-5,6,7,8-tetrahydropyrido[3,4-d]pyrimidin-2-yl)amino)propanoyl)piperidin-4-yl)methyl)benzyl)(methyl)amino)methyl)phenyl)-5-(2,4-dihydroxy-5-methylphenyl)-N-(2,2,2-trifluoroethyl)-4H-1,2,4-triazole-3-carboxamide, trifluoroacetic acid

¹H NMR (400 MHz, DMSO-d₆): δ 9.85-9.62 (m, 3H), 7.99 (d, J=8.4 Hz, 1H), 7.69 (d, J=8.4 Hz, 1H), 7.55-7.23 (m, 11H), 6.90 (s, 1H), 6.81-6.74 (m, 2H), 6.26 (s, 1H), 4.52-4.33 (m, 4H), 4.25-4.13 (m, 5H), 4.01-3.85 (m, 7H), 3.68-3.56 (m, 7H), 3.29-3.20 (m, 2H), 3.00-2.86 (m, 3H), 2.70-2.60 (m, 3H), 2.55-2.52 (m, 4H), 2.05 (s, 3H), 1.88 (s, 3H), 1.83-1.71 (m, 1H), 1.65-1.53 (m, 2H), 1.16-1.95 (m, 2H). LCMS (ESI): m/z found 1095.3[M−CF3COOH+H]⁺.

### Compound 115

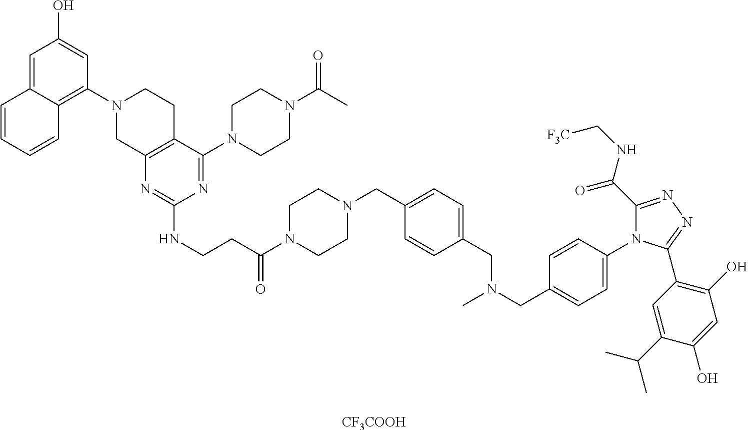 [SMILES: CC(=O)N1CCN(c2nc(NCCC(=O)N3CCN(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cc(O)cc4ccccc24)C3)CC1 ; "CF3COOH"]

### 4-(4-(((4-((4-(3-((4-(4-acetylpiperazin-1-yl)-7-(3-hydroxynaphthalen-1-yl)-5,6,7,8-tetrahydropyrido[3,4-d]pyrimidin-2-yl)amino)propanoyl)piperazin-1-yl)methyl)benzyl)(methyl)amino)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆): δ 9.80-9.65 (m, 2H), 7.99 (d, J=8.8 Hz, 1H), 7.75-7.25 (m, 11H), 6.89 (s, 1H), 6.75 (s, 1H), 6.30 (s, 1H), 4.52-4.41 (m, 2H), 4.32-4.41 (m, 3H), 4.17-4.09 (m, 3H), 4.00-3.90 (m, 4H), 3.61-3.53 (m, 17H), 3.30-3.20 (m, 4H), 3.02-2.82 (m, 6H), 2.72-2.64 (m, 4H), 2.05 (s, 3H), 0.95-0.85 (m, 6H). LCMS (ESI): m/z found 1124.3[M—CF3COOH+H]⁺.

### Compound 116

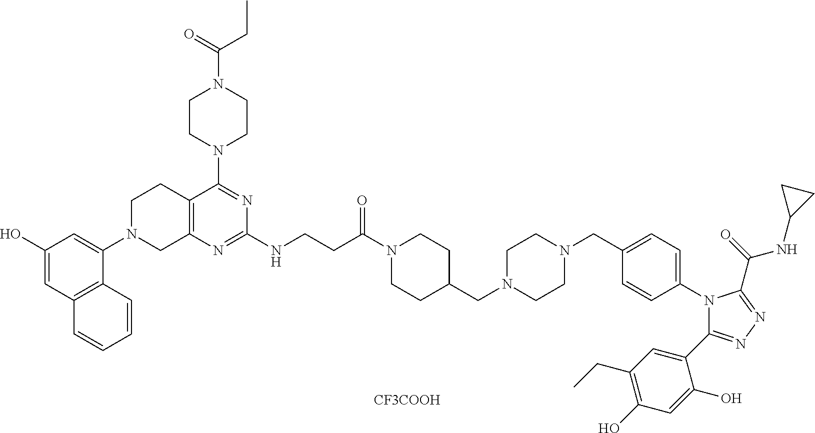 [SMILES: CCC(=O)N1CCN(c2nc(NCCC(=O)N3CCC(CN4CCN(Cc5ccc(-n6c(C(=O)NC7CC7)nnc6-c6cc(CC)c(O)cc6O)cc5)CC4)CC3)nc3c2CCN(c2cc(O)cc4ccccc24)C3)CC1 ; "CF3COOH"]

### N-cyclopropyl-5-(5-ethyl-2,4-dihydroxyphenyl)-4-(4-((4-((1-(3-((7-(3-hydroxynaphthalen-1-yl)-4-(4-propionylpiperazin-1-yl)-5,6,7,8-tetrahydropyrido[3,4-d]pyrimidin-2-yl)amino)propanoyl)piperidin-4-yl)methyl)piperazin-1-yl)methyl)phenyl)-4H-1,2,4-triazole-3-carboxamide, trifluoroacetic acid

¹H NMR (400 MHz, DMSO-d₆): δ 9.81-9.69 (m, 2H), 9.07 (d, J=4.4 Hz, 1H), 7.99 (d, J=8.4 Hz, 1H), 7.69 (d, J=8.4 Hz, 2H), 7.46-4.25 (m, 6H), 6.90 (s, 1H), 6.75 (s, 1H), 6.63 (s, 1H), 6.30 (s, 1H), 4.43-4.35 (m, 2H), 4.20-4.13 (m, 3H), 3.94-3.76 (m, 7H), 3.68-3.56 (m, 8H), 3.29-3.20 (m, 2H), 3.07-2.86 (m, 7H), 2.76-2.61 (m, 3H), 2.40-2.33 (m, 4H), 2.30-2.18 (m, 4H), 1.81-1.69 (m, 3H), 1.05-0.98 (m, 4H), 0.89-0.84 (m, 3H), 0.66-0.57 (m, 4H). LCMS (ESI): m/z found 1046.4 [M−CF3COOH+H]⁺.

### Compound 117

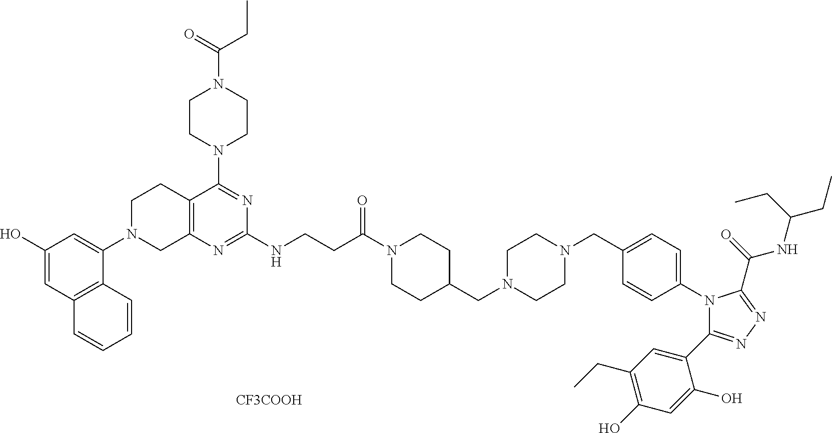 [SMILES: CCC(=O)N1CCN(c2nc(NCCC(=O)N3CCC(CN4CCN(Cc5ccc(-n6c(C(=O)NC(CC)CC)nnc6-c6cc(CC)c(O)cc6O)cc5)CC4)CC3)nc3c2CCN(c2cc(O)cc4ccccc24)C3)CC1 ; "CF3COOH"]

### 5-(5-ethyl-2,4-dihydroxyphenyl)-4-(4-((4-((1-(3-((7-(3-hydroxynaphthalen-1-yl)-4-(4-propionylpiperazin-1-yl)-5,6,7,8-tetrahydropyrido[3,4-d]pyrimidin-2-yl)amino)propanoyl)piperidin-4-yl)methyl)piperazin-1-yl)methyl)phenyl)-N-(pentan-3-yl)-4H-1,2,4-triazole-3-carboxamide, trifluoroacetic acid

¹H NMR (400 MHz, DMSO-d₆): δ 9.74 (d, J=27.7 Hz, 1H), 8.65 (d, J=9.2 Hz, 1H), 7.99 (d, J=8.0 Hz, 1H), 7.69 (d, J=8.8 Hz, 1H), 7.47-7.23 (m, 5H), 6.90 (s, 1H), 6.75 (d, J=1.6 Hz, 1H), 6.70-6.61 (m, 1H), 6.31 (s, 1H), 4.43-4.35 (m, 1H), 4.15 (s, 2H), 3.99-3.76 (m, 9H), 3.71-3.47 (m, 14H), 3.25 (s, 3H), 3.10-2.83 (m, 8H), 2.71-2.61 (m, 2H), 2.42-2.19 (m, 6H), 1.84-1.67 (m, 2H), 1.55-1.34 (m, 4H), 1.02 (t, J=7.4 Hz, 4H), 0.93-0.84 (m, 3H), 0.78 (t, J=7.3 Hz, 6H). LCMS (ESI): m/z found 1076.4[M−CF3COOH+H]⁺.

### Compound 118

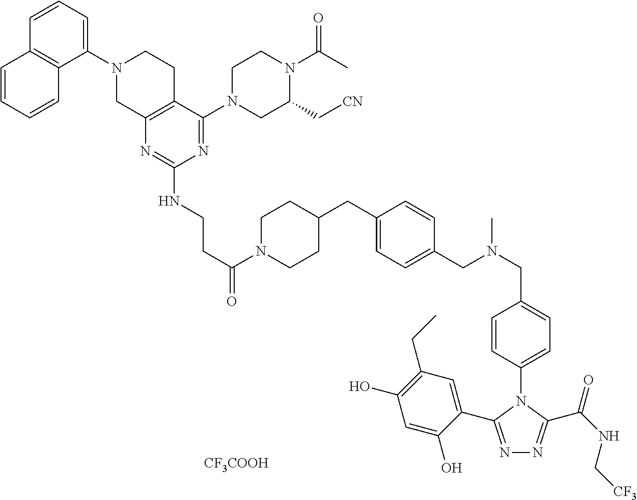 [SMILES: CCc1cc(-c2nnc(C(=O)NCC(F)(F)F)n2-c2ccc(CN(C)Cc3ccc(CC4CCN(C(=O)CCNc5nc6c(c(N7CCN(C(C)=O)[C@H](CC#N)C7)n5)CCN(c5cccc7ccccc57)C6)CC4)cc3)cc2)c(O)cc1O ; "CF3COOH"]

### (R)-4-(4-(((4-((1-(3-((4-(4-acetyl-3-(cyanomethyl)piperazin-1-yl)-7-(naphthalen-1-yl)-5,6,7,8-tetrahydropyrido[3,4-d]pyrimidin-2-yl)amino)propanoyl)piperidin-4-yl)methyl)benzyl)(methyl)amino)methyl)phenyl)-5-(5-ethyl-2,4-dihydroxyphenyl)-N-(2,2,2-trifluoroethyl)-4H-1,2,4-triazole-3-carboxamide, trifluoroacetic acid

¹H NMR (400 MHz, CD₃OD) δ 8.22 (d, J=8.9 Hz, 1H), 7.91-7.85 (m, 1H), 7.65 (d, J=8.3 Hz, 1H), 7.58-7.48 (m, 5H), 7.47-7.44 (m, 2H), 7.38 (d, J=8.0 Hz, 2H), 7.32 (d, J=8.0 Hz, 2H), 7.23 (d, J=7.4 Hz, 1H), 6.78 (s, 1H), 6.24 (s, 1H), 5.02-4.93 (m, 2H), 4.75-4.36 (m, 7H), 4.23 (s, 3H), 4.03-3.92 (m, 3H), 3.81-3.64 (m, 4H), 3.16-2.87 (m, 7H), 2.82-2.67 (m, 6H), 2.66-2.58 (m, 2H), 2.40-2.25 (m, 3H), 2.21-2.16 (m, 2H), 1.86 (s, 1H), 1.75-1.61 (m, 2H), 1.39-1.12 (m, 6H), 0.95 (t, J=7.6 Hz, 3H). LCMS (ESI): m/z found 1132.5[M—CF3COOH+H]⁺.

### Compound 119

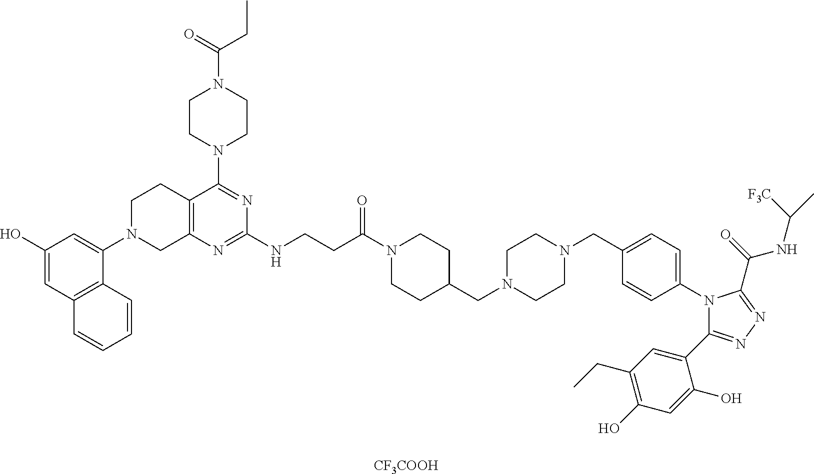 [SMILES: CCC(=O)N1CCN(c2nc(NCCC(=O)N3CCC(CN4CCN(Cc5ccc(-n6c(C(=O)NC(C)C(F)(F)F)nnc6-c6cc(CC)c(O)cc6O)cc5)CC4)CC3)nc3c2CCN(c2cc(O)cc4ccccc24)C3)CC1 ; "CF3COOH"]

### 5-(5-ethyl-2,4-dihydroxyphenyl)-4-(4-((4-((1-(3-((7-(3-hydroxynaphthalen-1-yl)-4-(4-propionylpiperazin-1-yl)-5,6,7,8-tetrahydropyrido[3,4-d]pyrimidin-2-yl)amino)propanoyl)piperidin-4-yl)methyl)piperazin-1-yl)methyl)phenyl)-N-(1,1,1-trifluoropropan-2-yl)-4H-1,2,4-triazole-3-carboxamide, trifluoroacetic acid

¹H NMR (400 MHz, DMSO-d₆): δ 9.93-9.53 (m, 3H), 7.99 (d, J=8.4 Hz, 1H), 7.88-7.66 (m, 2H), 7.55-7.20 (m, 6H), 6.90 (s, 1H), 6.79-6.65 (m, 2H), 6.30 (s, 1H), 4.71-4.58 (m, 1H), 4.39 (d, J=14.4 Hz, 1H), 4.16 (s, 2H), 3.96-3.72 (m, 6H), 3.70-3.37 (m, 10H), 3.25 (s, 2H), 3.11-2.83 (m, 7H), 2.72-2.56 (m, 2H), 2.45-2.21 (m, 8H), 2.09-1.95 (m, 1H), 1.75 (d, J=11.6 Hz, 3H), 1.34 (d, J=7.0 Hz, 3H), 1.19-1.07 (m, 1H), 1.02 (t, J=7.4 Hz, 4H), 0.89 (t, J=7.4 Hz, 3H). LCMS (ESI): m/z found 1103.0[M−CF3COOH+H]⁺.

### Compound 120

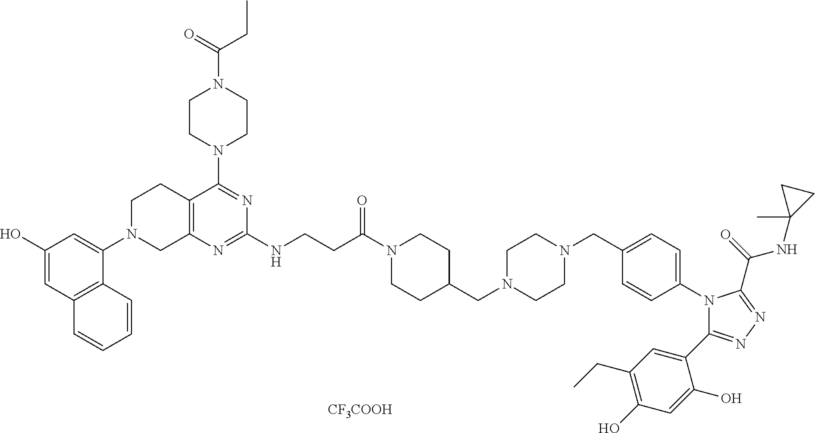 [SMILES: CCC(=O)N1CCN(c2nc(NCCC(=O)N3CCC(CN4CCN(Cc5ccc(-n6c(C(=O)NC7(C)CC7)nnc6-c6cc(CC)c(O)cc6O)cc5)CC4)CC3)nc3c2CCN(c2cc(O)cc4ccccc24)C3)CC1 ; "CF3COOH"]

### 5-(5-ethyl-2,4-dihydroxyphenyl)-4-(4-((4-((1-(3-((7-(3-hydroxynaphthalen-1-yl)-4-(4-propionylpiperazin-1-yl)-5,6,7,8-tetrahydropyrido[3,4-d]pyrimidin-2-yl)amino)propanoyl)piperidin-4-yl)methyl)piperazin-1-yl)methyl)phenyl)-N-(1-methylcyclopropyl)-4H-1,2,4-triazole-3-carboxamide, trifluoroacetic acid

¹H NMR (400 MHz, DMSO-d₆) δ 10.34 (s, 1H), 9.70 (s, 1H), 9.64 (s, 1H), 9.09 (s, 1H), 8.00 (d, J=8.5 Hz, 1H), 7.66 (d, J=9.0 Hz, 1H), 7.32 (m, 7H), 6.84 (s, 1H), 6.74 (s, 1H), 6.55 (s, 2H), 6.31 (s, 1H), 4.36 (d, J=11.8 Hz, 1H), 3.97 (s, 3H), 3.57-3.56 (m, 4H), 3.47-3.45 (m, 6H), 3.31 (s, 3H), 3.23-3.20 (m, 2H), 2.96-2.95 (m, 1H), 2.79 (s, 2H), 2.36 (d, J=7.4 Hz, 9H), 2.21 (d, J=7.4 Hz, 3H), 2.10 (s, 3H), 1.67 (m, 2H), 1.23 (s, 4H), 1.01 (m, 5H), 0.83-0.82 (m, 5H), 0.63-0.62 (m, 2H), 0.52-0.51 (m, 2H). LCMS (ESI): m/z found 1060.4 [M—CF3COOH+H]⁺.

### Compound 121

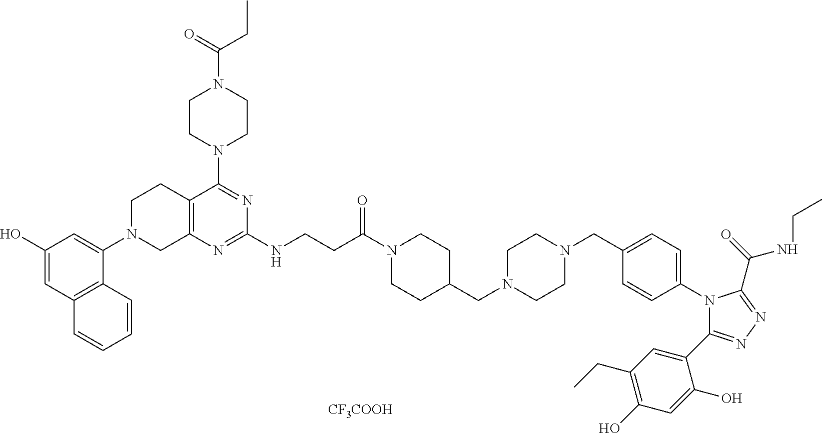 [SMILES: CCNC(=O)c1nnc(-c2cc(CC)c(O)cc2O)n1-c1ccc(CN2CCN(CC3CCN(C(=O)CCNc4nc5c(c(N6CCN(C(=O)CC)CC6)n4)CCN(c4cc(O)cc6ccccc46)C5)CC3)CC2)cc1 ; "CF3COOH"]

### N-ethyl-5-(5-ethyl-2,4-dihydroxyphenyl)-4-(4-((4-((1-(3-((7-(3-hydroxynaphthalen-1-yl)-4-(4-propionylpiperazin-1-yl)-5,6,7,8-tetrahydropyrido[3,4-d]pyrimidin-2-yl)amino)propanoyl)piperidin-4-yl)methyl)piperazin-1-yl)methyl)phenyl)-4H-1,2,4-triazole-3-carboxamide, trifluoroacetic acid

¹H NMR (400 MHz, DMSO-d₆) δ 9.73 (s, 2H), 9.01 (t, J=5.8 Hz, 1H), 7.99 (d, J=8.3 Hz, 1H), 7.86 (s, 1H), 7.69 (d, J=8.3 Hz, 1H), 7.41 (t, J=7.4 Hz, 3H), 7.37-7.26 (m, 3H), 6.90 (s, 1H), 6.76 (s, 1H), 6.62 (s, 1H), 6.31 (s, 1H), 4.39 (d, J=13.0 Hz, 1H), 4.17 (s, 2H), 3.88 (d, J=12.8 Hz, 7H), 3.63 (s, 8H), 3.31-3.13 (m, 6H), 2.97 (d, J=28.2 Hz, 8H), 2.74-2.53 (m, 6H), 2.37 (dd, J=14.9, 7.4 Hz, 4H), 2.25 (dd, J=14.8, 7.4 Hz, 3H), 1.99 (s, 1H), 1.75 (d, J=9.2 Hz, 2H), 1.13-0.95 (m, 9H), 0.87 (t, J=7.4 Hz, 3H). LCMS (ESI): m/z found 1034.4 [M−CF3COOH+H]⁺.

### Compound 122

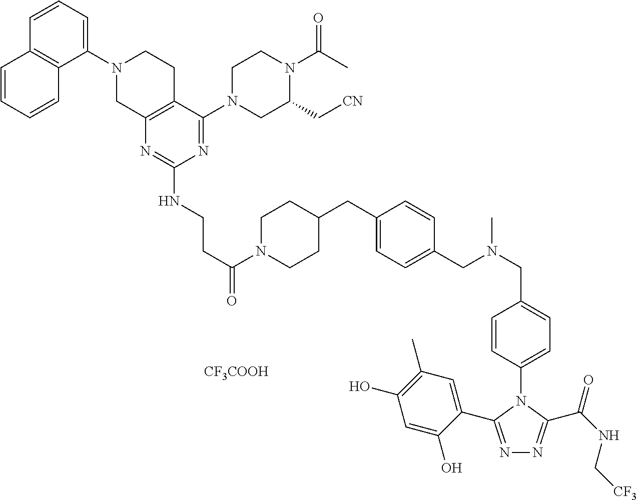 [SMILES: CC(=O)N1CCN(c2nc(NCCC(=O)N3CCC(Cc4ccc(CN(C)Cc5ccc(-n6c(C(=O)NCC(F)(F)F)nnc6-c6cc(C)c(O)cc6O)cc5)cc4)CC3)nc3c2CCN(c2cccc4ccccc24)C3)C[C@H]1CC#N ; "CF3COOH"]

### (R)-4-(4-(((4-((1-(3-((4-(4-acetyl-3-(cyanomethyl)piperazin-1-yl)-7-(naphthalen-1-yl)-5,6,7,8-tetrahydropyrido[3,4-d]pyrimidin-2-yl)amino)propanoyl)piperidin-4-yl)methyl)benzyl)(methyl)amino)methyl)phenyl)-5-(2,4-dihydroxy-5-methylphenyl)-N-(2,2,2-trifluoroethyl)-4H-1,2,4-triazole-3-carboxamide, trifluoroacetic acid

¹H NMR (400 MHz, DMSO-d₆) δ 10.08 (s, 1H), 9.83 (s, 1H), 9.60 (t, J=6.3 Hz, 1H), 8.18 (s, 1H), 7.97-7.85 (m, 1H), 7.62 (d, J=8.2 Hz, 1H), 7.53 (s, 2H), 7.44 (t, J=7.8 Hz, 1H), 7.35 (d, J=8.2 Hz, 2H), 7.22 (dd, J=19.3, 8.2 Hz, 5H), 7.10 (d, J=7.6 Hz, 2H), 6.64 (s, 1H), 6.55 (s, 1H), 6.38 (s, 1H), 4.91 (s, 1H), 4.53 (s, 1H), 4.36 (d, J=12.2 Hz, 2H), 4.13-3.72 (m, 8H), 3.46 (d, J=20.4 Hz, 6H), 3.17-2.76 (m, 8H), 2.68 (s, 1H), 2.44 (t, J=10.6 Hz, 3H), 2.21-2.00 (m, 6H), 1.79 (s, 3H), 1.71 (s, 1H), 1.55 (d, J=11.8 Hz, 2H), 1.23 (s, 3H), 1.00 (dd, J=35.9, 11.1 Hz, 2H). LCMS (ESI): m/z found 1118.5 [M−CF3COOH+H]⁺.

### Compound 123

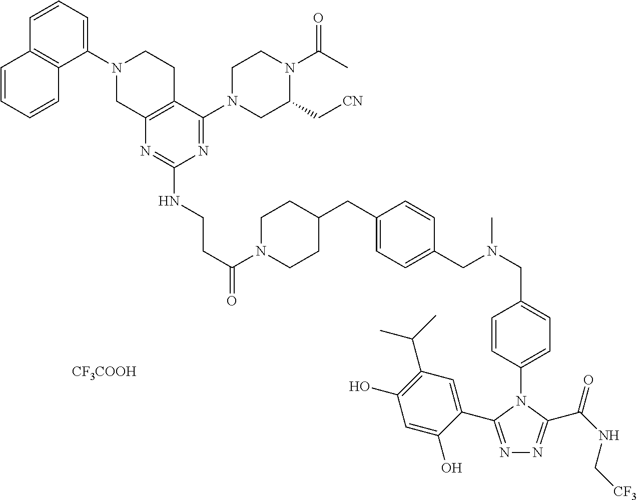 [SMILES: CC(=O)N1CCN(c2nc(NCCC(=O)N3CCC(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cccc4ccccc24)C3)C[C@H]1CC#N ; "CF3COOH"]

### (R)-4-(4-(((4-((1-(3-((4-(4-acetyl-3-(cyanomethyl)piperazin-1-yl)-7-(naphthalen-1-yl)-5,6,7,8-tetrahydropyrido[3,4-d]pyrimidin-2-yl)amino)propanoyl)piperidin-4-yl)methyl)benzyl)(methyl)amino)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆) δ 9.97 (s, 1H), 9.80-9.60 (m, 2H), 8.17 (s, 1H), 7.99-7.91 (m, 1H), 7.87-7.17 (m, 13H), 6.76 (s, 1H), 6.30 (s, 1H), 4.85 (s, 1H), 4.62-4.15 (m, 10H), 4.04-3.78 (m, 5H), 3.36-3.11 (m, 4H), 3.10-2.87 (m, 5H), 2.70-2.52 (m, 8H), 2.23-1.94 (m, 4H), 1.85-1.71 (m, 1H), 1.67-1.50 (m, 2H), 1.37-1.06 (m, 4H), 1.05-0.82 (m, 7H). LCMS (ESI): m/z found 1146.5[M−CF3COOH+H]⁺.

### Compound 124

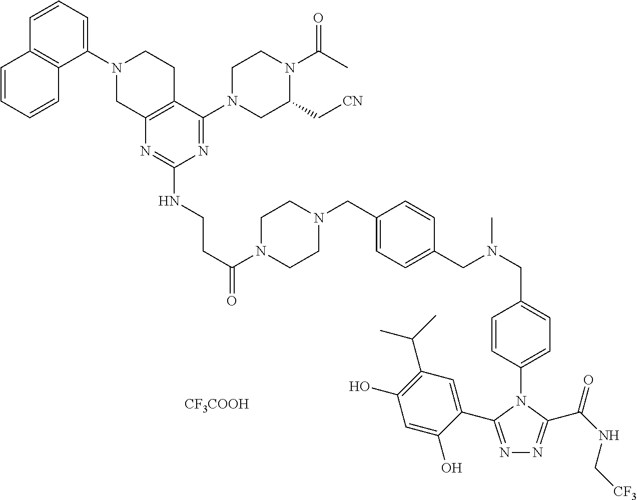 [SMILES: CC(=O)N1CCN(c2nc(NCCC(=O)N3CCN(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cccc4ccccc24)C3)C[C@H]1CC#N ; "CF3COOH"]

### (R)-4-(4-(((4-((4-(3-((4-(4-acetyl-3-(cyanomethyl)piperazin-1-yl)-7-(naphthalen-1-yl)-5,6,7,8-tetrahydropyrido[3,4-d]pyrimidin-2-yl)amino)propanoyl)piperazin-1-yl)methyl)benzyl)(methyl)amino)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, DMSO-d₆) δ 10.45 (s, 1H), 9.75 (s, 1H), 9.60 (t, J=6.0 Hz, 1H), 8.17 (s, 1H), 7.96-7.86 (m, 1H), 7.66-7.14 (m, 13H), 6.64-6.50 (m, 2H), 6.33 (s, 1H), 4.55-4.30 (m, 1H), 4.10-3.74 (m, 6H), 3.56-3.41 (m, 9H), 3.10-2.79 (m, 7H), 2.75-2.60 (m, 1H), 2.60-2.51 (m, 3H), 2.31 (s, 4H), 2.18-1.93 (m, 7H), 1.23 (s, 5H), 0.77 (d, J=6.8 Hz, 6H). LCMS (ESI): m/z found 1147.6[M−CF3COOH+H]⁺.

### Compound 125

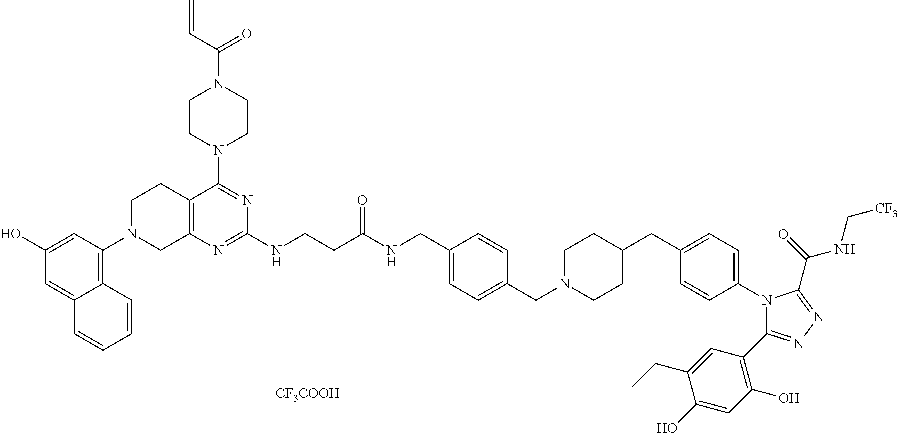 [SMILES: C=CC(=O)N1CCN(c2nc(NCCC(=O)NCc3ccc(CN4CCC(Cc5ccc(-n6c(C(=O)NCC(F)(F)F)nnc6-c6cc(CC)c(O)cc6O)cc5)CC4)cc3)nc3c2CCN(c2cc(O)cc4ccccc24)C3)CC1 ; "CF3COOH"]

### 4-(4-((1-(4-((3-((4-(4-acryloylpiperazin-1-yl)-7-(3-hydroxynaphthalen-1-yl)-5,6,7,8-tetrahydropyrido[3,4-d]pyrimidin-2-yl)amino)propanamido)methyl)benzyl)piperidin-4-yl)methyl)phenyl)-5-(5-ethyl-2,4-dihydroxyphenyl)-N-(2,2,2-trifluoroethyl)-4H-1,2,4-triazole-3-carboxamide, trifluoroacetic acid

¹H NMR (400 MHz, CD₃OD) δ 8.05 (s, 1H), 7.64 (d, J=8.8 Hz, 1H), 7.47-7.23 (m, 10H), 6.92-6.75 (m, 2H), 6.58 (s, 1H), 6.35-6.24 (m, 2H), 5.85-5.75 (m, 1H), 5.40-5.23 (m, 1H), 4.42 (s, 1H), 4.30-4.15 (m, 3H), 4.04-3.75 (m, 10H), 3.50-3.15 (m, 6H), 3.03-2.80 (m, 3H), 2.70-2.60 (m, 3H), 2.30-2.22 (m, 1H), 1.95-1.80 (m, 2H), 1.30 (s, 8H), 0.86 (t, J=7.4 Hz, 3H). LCMS (ESI): m/z found 1107.3[M—CF3COOH+H]⁺.

### Compound 126

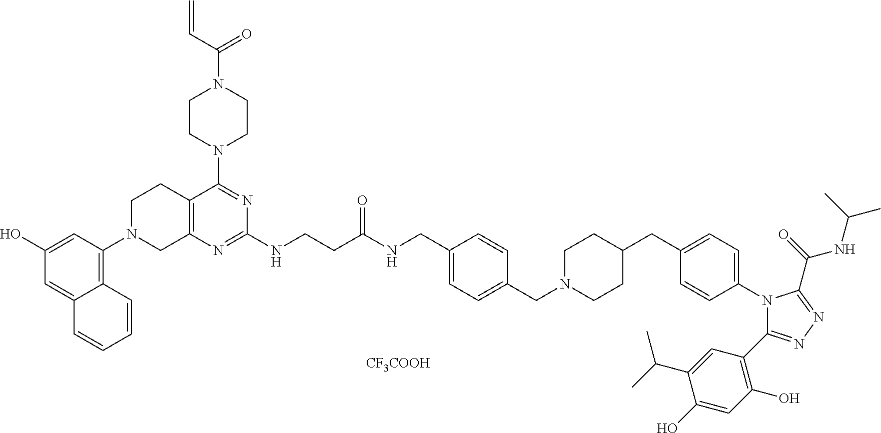 [SMILES: C=CC(=O)N1CCN(c2nc(NCCC(=O)NCc3ccc(CN4CCC(Cc5ccc(-n6c(C(=O)NC(C)C)nnc6-c6cc(C(C)C)c(O)cc6O)cc5)CC4)cc3)nc3c2CCN(c2cc(O)cc4ccccc24)C3)CC1 ; "CF3COOH"]

### 4-(4-((1-(4-((3-((4-(4-acryloylpiperazin-1-yl)-7-(3-hydroxynaphthalen-1-yl)-5,6,7,8-tetrahydropyrido[3,4-d]pyrimidin-2-yl)amino)propanamido)methyl)benzyl)piperidin-4-yl)methyl)phenyl)-5-(2,4-dihydroxy-5-isopropylphenyl)-N-isopropyl-4H-1,2,4-triazole-3-carboxamide, trifluoroacetic acid

¹H NMR (400 MHz, DMSO-d₆) δ 9.90-9.74 (m, 2H), 9.61-9.37 (m, 1H), 8.80 (s, 1H), 8.54 (s, 1H), 8.00 (d, J=8.2 Hz, 1H), 7.69 (d, J=8.4 Hz, 1H), 7.50-7.25 (m, 9H), 6.94-6.74 (m, 2H), 6.63-6.54 (m, 1H), 6.34 (s, 1H), 6.28-6.10 (m, 1H), 5.75 (d, J=10.7 Hz, 1H), 4.35-4.12 (m, 7H), 3.94-3.60 (m, 20H), 3.35-3.23 (s, 3H), 2.97-2.82 (m, 4H), 2.38-2.30 (m, 3H), 1.81-1.68 (m, 2H), 1.45-1.37 (m, 1H), 1.24 (s, 1H), 1.13-1.02 (m, 4H), 0.81 (d, J=6.8 Hz, 6H). LCMS (ESI): m/z found 1081.5[M—CF3COOH+H]⁺.

### Compound 127

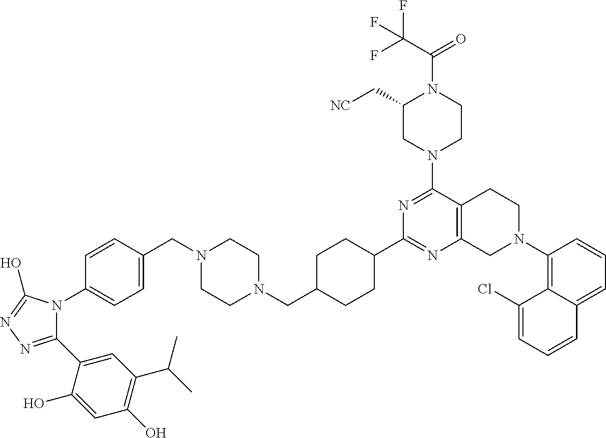 [SMILES: CC(C)c1cc(-c2nnc(O)n2-c2ccc(CN3CCN(CC4CCC(c5nc6c(c(N7CCN(C(=O)C(F)(F)F)[C@@H](CC#N)C7)n5)CCN(c5cccc7cccc(Cl)c57)C6)CC4)CC3)cc2)c(O)cc1O]

### (S)-2-(4-(7-(8-chloronaphthalen-1-yl)-2-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-5,6,7,8-tetrahydropyrido[3,4-d]pyrimidin-4-yl)-1-(2,2,2-trifluoroacetyl)piperazin-2-yl)acetonitrile

¹H NMR (CD3OD, 400 MHz): δ 7.71 (d, J=8.0 Hz, 1H), 7.55 (dd, J=8.0, 2.0 Hz, 1H), 7.43-7.34 (m, 4H), 7.28-7.14 (m, 4H), 6.63 (s, 1H), 6.16 (s, 1H), 4.58-3.21 (m, 13H), 3.06-2.69 (m, 15H), 2.54-2.43 (m, 4H), 2.16-2.09 (m, 1H), 1.89-1.84 (m, 2H), 1.07-0.98 (m, 2H), 0.82 (d, J=6.8 Hz, 6H); LC-MS: m/z 1019.4 [M+H]⁺.

### Compound 128

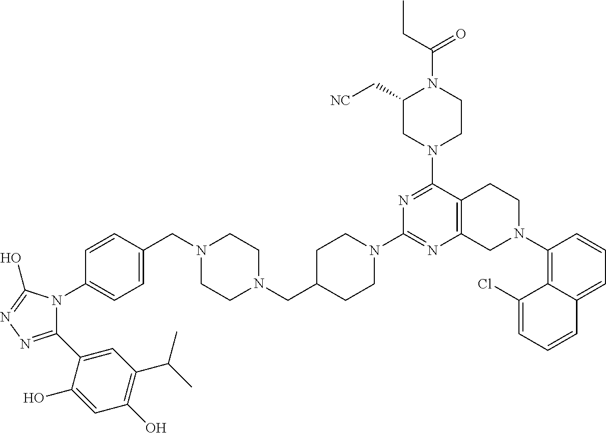 [SMILES: CCC(=O)N1CCN(c2nc(N3CCC(CN4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(4-(7-(8-chloronaphthalen-1-yl)-2-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-5,6,7,8-tetrahydropyrido[3,4-d]pyrimidin-4-yl)-1-propionylpiperazin-2-yl)acetonitrile

¹H NMR (CD₃OD, 400 MHz): δ 7.83 (d, J=8.0 Hz, 1H), 7.67 (dd, J=8.0 Hz, 3.6 Hz, 1H), 7.55-7.45 (m, 4H), 7.38 (t, J=8.0 Hz, 1H), 7.34-7.26 (m, 4H), 6.75 (s, 1H), 6.28 (s, 1H), 5.06-4.89 (m, 1H), 4.70-4.53 (m, 3H), 4.16 (dd, J=16.8 Hz, 5.2 Hz, 1H), 4.07-3.43 (m, 8H), 3.23-2.82 (m, 10H), 2.66-2.47 (m, 11H), 2.34 (d, J=7.2 Hz, 2H), 1.87-1.78 (m, 3H), 1.19-1.15 (m, 3H), 0.94 (d, J=7.2 Hz, 6H). LC-MS: m/z 979.4 [M+H]⁺.

### Compound 129

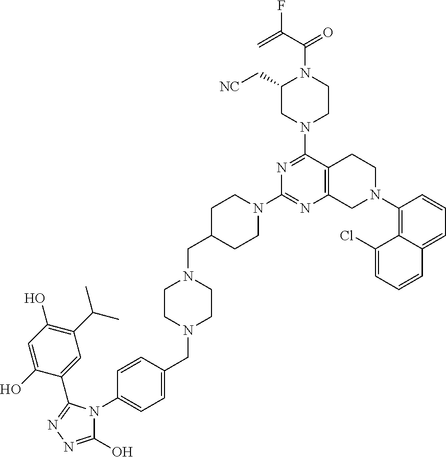 [SMILES: C=C(F)C(=O)N1CCN(c2nc(N3CCC(CN4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(4-(7-(8-chloronaphthalen-1-yl)-2-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-5,6,7,8-tetrahydropyrido[3,4-d]pyrimidin-4-yl)-1-(2-fluoroacryloyl)piperazin-2-yl)acetonitrile

¹H NMR (CD3OD, 400 MHz): δ 7.76 (d, J=8.0 Hz, 1H), 7.64 (d, J=8.8 Hz, 1H), 7.49-7.41 (m, 4H), 7.32-7.22 (m, 4H), 6.69 (s, 1H), 6.15 (s, 1H), 5.32-5.21 (m, 2H), 4.47-3.37 (m, 12H), 3.17-2.86 (m, 16H), 2.66-2.58 (m, 4H), 2.01-1.96 (m, 1H), 1.89-1.83 (m, 2H), 1.28-1.21 (m, 2H), 0.85 (d, J=6.8 Hz, 6H); LC-MS: m/z 665.4 [M+H]⁺.

### Compound 130

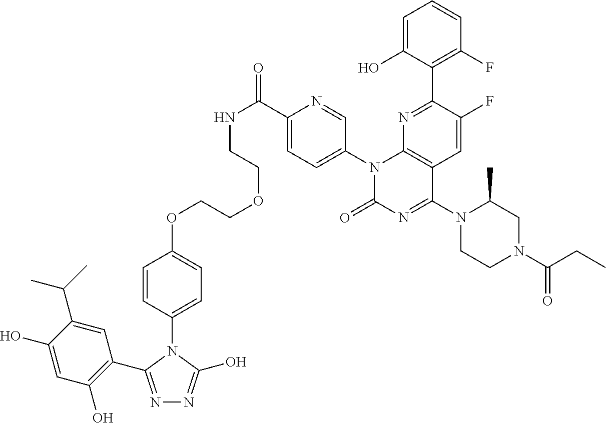 [SMILES: CCC(=O)N1CCN(c2nc(=O)n(-c3ccc(C(=O)NCCOCCOc4ccc(-n5c(O)nnc5-c5cc(C(C)C)c(O)cc5O)cc4)nc3)c3nc(-c4c(O)cccc4F)c(F)cc23)[C@@H](C)C1]

### N-(2-(2-(4-(3-(2,4-dihydroxy-5-isopropylphenyl)-5-hydroxy-4H-1,2,4-triazol-4-yl)phenoxy)ethoxy)ethyl)-5-(6-fluoro-7-(2-fluoro-6-hydroxyphenyl)-4-((S)-2-methyl-4-propionylpiperazin-1-yl)-2-oxopyrido[2,3-d]pyrimidin-1(2H)-yl)picolinamide

¹H NMR (DMSO-d₆, 400 MHz): δ 11.89 (s, 1H), 10.13 (s, 1H), 9.62 (s, 1H), 9.48 (s, 1H), 8.78 (t, J=6.0 Hz, 1H), 8.58 (s, 1H), 8.26 (dd, J=8.8 Hz, 1H), 8.12 (d, J=7.6 Hz, 1H), 7.97-7.94 (m, 1H), 7.29-7.23 (m, 1H), 7.08 (d, J=9.2 Hz, 2H), 6.92 (d, J=8.8 Hz, 2H), 6.82 (s, 1H), 6.74-6.68 (m, 2H), 6.25 (s, 1H), 4.86 (s, 1H) 4.369-4.23 (m, 2H), 4.08-3.38 (m, 11H), 3.14-2.95 (m, 2H), 2.41-2.34 (m, 2H), 1.39-1.23 (m, 3H), 1.07-0.97 (m, 9H). LC-MS: m/z 947.4 [M+H]⁺.

### Compound 131

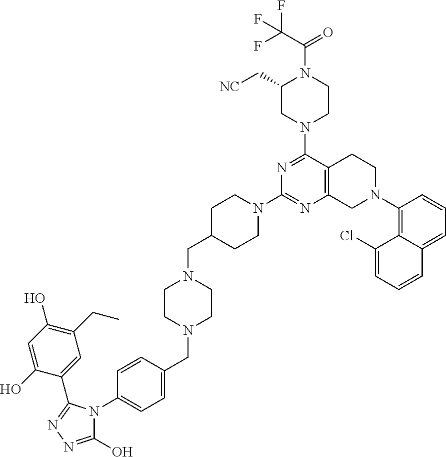 [SMILES: CCc1cc(-c2nnc(O)n2-c2ccc(CN3CCN(CC4CCN(c5nc6c(c(N7CCN(C(=O)C(F)(F)F)[C@@H](CC#N)C7)n5)CCN(c5cccc7cccc(Cl)c57)C6)CC4)CC3)cc2)c(O)cc1O]

### (S)-2-(4-(7-(8-chloronaphthalen-1-yl)-2-(4-((4-(4-(3-(5-ethyl-2,4-dihydroxyphenyl)-5-hydroxy-4H-1,2,4-triazol-4-yl)benzyl)piperazin-1-yl)methyl)piperidin-1-yl)-5,6,7,8-tetrahydropyrido[3,4-d]pyrimidin-4-yl)-1-(2,2,2-trifluoroacetyl)piperazin-2-yl)acetonitrile

¹H NMR (CD3OD, 400 MHz): δ 7.71 (d, J=8.0 Hz, 1H), 7.56-7.54 (m, 1H), 7.43-7.17 (m, 6H), 7.13 (d, J=8.0 Hz, 2H), 6.63 (s, 1H), 6.13 (s, 1H), 4.93-4.85 (m, 1H), 4.58-4.55 (m, 2H), 4.16 (d, J=16.8 Hz, 1H), 3.98-3.42 (m, 8H), 3.06-2.69 (m, 9H), 2.55-2.37 (m, 8H), 2.25 (q, J=8.0 Hz, 2H), 2.21-2.11 (m, 2H), 1.94-1.62 (m, 3H), 1.070.98 (m, 2H), 0.85 (t, J=7.8 Hz, 3H). LC-MS: m/z 1005.4 [M+H]⁺.

### Compound 132

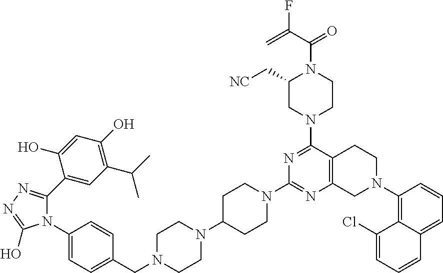 [SMILES: C=C(F)C(=O)N1CCN(c2nc(N3CCC(N4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(4-(7-(8-chloronaphthalen-1-yl)-2-(4-(4-(4-(3-(2,4-dihydroxy-5-isopropylphenyl)-5-hydroxy-4H-1,2,4-triazol-4-yl)benzyl)piperazin-1-yl)piperidin-1-yl)-5,6,7,8-tetrahydropyrido[3,4-d]pyrimidin-4-yl)-1-(2-fluoroacryloyl)piperazin-2-yl)acetonitrile

¹H NMR (CD3OD, 400 MHz): δ 7.83 (d, J=8.0 Hz, 1H), 7.67 (dd, J=8.0, 2.8 Hz, 1H), 7.55-7.45 (m, 4H), 7.40-7.25 (m, 4H), 6.75 (s, 1H), 6.28 (s, 1H), 5.38-5.33 (m, 2H), 4.83-4.76 (m, 2H), 4.31-3.50 (m, 11H), 3.18-2.53 (m, 16H), 1.99-1.94 (m, 2H), 1.52-1.32 (m, 4H), 0.94 (d, J=6.8 Hz, 6H); LC-MS: m/z 1006.1 [M+H]⁺.

### Compound 133

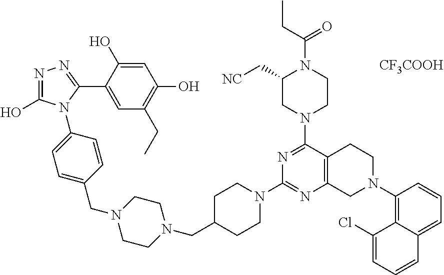 [SMILES: CCC(=O)N1CCN(c2nc(N3CCC(CN4CCN(Cc5ccc(-n6c(O)nnc6-c6cc(CC)c(O)cc6O)cc5)CC4)CC3)nc3c2CCN(c2cccc4cccc(Cl)c24)C3)C[C@@H]1CC#N ; "CF3COOH"]

### (S)-2-(4-(7-(8-chloronaphthalen-1-yl)-2-(4-((4-(4-(3-(5-ethyl-2,4-dihydroxyphenyl)-5-hydroxy-4H-1,2,4-triazol-4-yl)benzyl)piperazin-1-yl)methyl)piperidin-1-yl)-5,6,7,8-tetrahydropyrido[3,4-d]pyrimidin-4-yl)-1-propionylpiperazin-2-yl)acetonitrile, trifluoroacetic acid

¹H NMR (CD₃OD, 400 MHz): δ 7.87 (d, J=8.0 Hz, 1H), 7.75 (dd, J=8.0 Hz, 2.4 Hz, 1H), 7.60-7.50 (m, 4H), 7.42 (t, J=8.0 Hz, 1H), 7.39-7.32 (m, 4H), 6.82 (s, 1H), 6.24 (s, 1H), 5.02-4.90 (m, 1H), 4.79-4.73 (m, 1H), 4.57-4.39 (m, 5H), 4.02-3.49 (m, 8H), 3.32-2.86 (m, 14H), 2.76 (s, 2H), 2.52-2.48 (m, 2H), 2.41 (q, J=7.6 Hz, 2H), 2.11 (s, 1H), 1.98-1.94 (m, 2H), 1.39-1.29 (m, 2H), 1.19-1.15 (m, 3H), 1.00 (t, J=7.6 Hz, 3H). LC-MS: m/z 965.5 [M-CF₃COOH+H]⁺.

### Compound 134

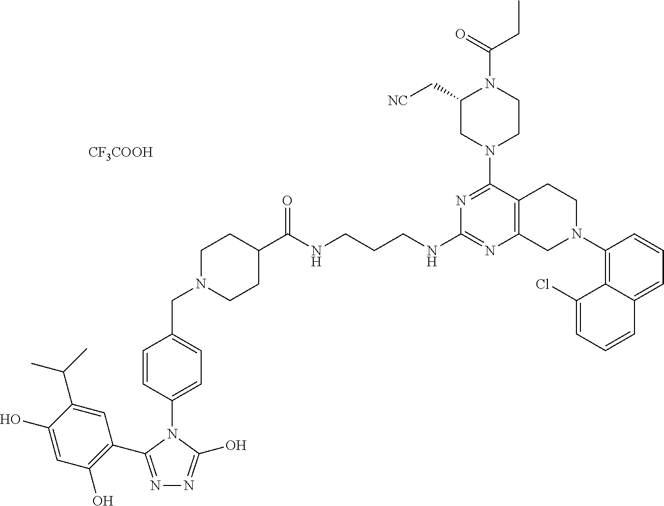 [SMILES: CCC(=O)N1CCN(c2nc(NCCCNC(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N ; "CF3COOH"]

### (S)—N-(3-((7-(8-chloronaphthalen-1-yl)-4-(3-(cyanomethyl)-4-propionylpiperazin-1-yl)-5,6,7,8-tetrahydropyrido[3,4-d]pyrimidin-2-yl)amino)propyl)-1-(4-(3-(2,4-dihydroxy-5-isopropylphenyl)-5-hydroxy-4H-1,2,4-triazol-4-yl)benzyl)piperidine-4-carboxamide, trifluoroacetic acid

¹H NMR (CD₃OD, 400 MHz): δ 7.86 (d, J=8.0 Hz, 1H), 7.75 (dd, J=8.0 Hz, 2.4 Hz, 1H), 7.59-7.50 (m, 4H), 7.43-7.36 (m, 4H), 6.89 (s, 1H), 6.24 (s, 1H), 5.04-4.92 (m, 1H), 4.78-4.31 (m, 7H), 4.01-3.50 (m, 9H), 3.30-2.49 (m, 13H), 2.08-1.85 (m, 6H), 1.18-1.14 (m, 3H), 1.03 (d, J=7.2 Hz, 6H). LC-MS: m/z 981.5 [M-CF₃COOH+H]⁺.

### Compound 135

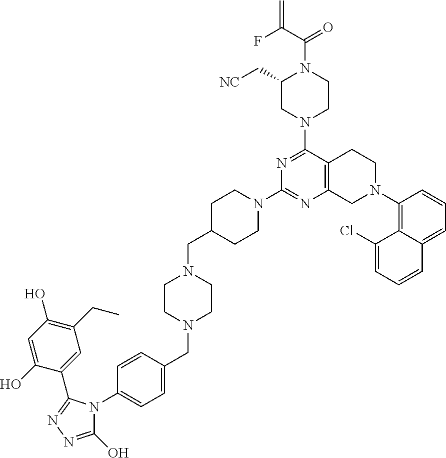 [SMILES: C=C(F)C(=O)N1CCN(c2nc(N3CCC(CN4CCN(Cc5ccc(-n6c(O)nnc6-c6cc(CC)c(O)cc6O)cc5)CC4)CC3)nc3c2CCN(c2cccc4cccc(Cl)c24)C3)C[C@@H]1CC#N]

### (S)-2-(4-(7-(8-chloronaphthalen-1-yl)-2-(4-((4-(4-(3-(5-ethyl-2,4-dihydroxyphenyl)-5-hydroxy-4H-1,2,4-triazol-4-yl)benzyl)piperazin-1-yl)methyl)piperidin-1-yl)-5,6,7,8-tetrahydropyrido[3,4-d]pyrimidin-4-yl)-1-(2-fluoroacryloyl)piperazin-2-yl)acetonitrile

¹H NMR (CD3OD, 400 MHz): δ 7.87 (d, J=8.0 Hz, 1H), 7.75 (d, J=8.4 Hz, 1H), 7.60-7.50 (m, 4H), 7.44-7.33 (m, 4H), 6.82 (s, 1H), 6.24 (s, 1H), 5.43-5.32 (m, 2H), 4.558-4.4 (m, 5H), 4.02-3.50 (m, 9H), 3.17-2.71 (m, 17H), 2.41 (q, J=7.2 Hz, 2H), 2.15-2.06 (m, 1H), 1.97-1.95 (m, 2H), 1.38-1.29 (m, 2H), 1.00 (t, J=7.6 Hz, 3H); LC-MS: m/z 981.4 [M+H]⁺.

### Compound 136

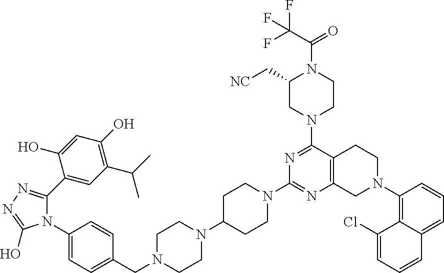 [SMILES: CC(C)c1cc(-c2nnc(O)n2-c2ccc(CN3CCN(C4CCN(c5nc6c(c(N7CCN(C(=O)C(F)(F)F)[C@@H](CC#N)C7)n5)CCN(c5cccc7cccc(Cl)c57)C6)CC4)CC3)cc2)c(O)cc1O]

### (S)-2-(4-(7-(8-chloronaphthalen-1-yl)-2-(4-(4-(4-(3-(2,4-dihydroxy-5-isopropylphenyl)-5-hydroxy-4H-1,2,4-triazol-4-yl)benzyl)piperazin-1-yl)piperidin-1-yl)-5,6,7,8-tetrahydropyrido[3,4-d]pyrimidin-4-yl)-1-(2,2,2-trifluoroacetyl)piperazin-2-yl)acetonitrile

¹H NMR (CD3OD, 400 MHz): δ 7.83 (d, J=8.0 Hz, 1H), 7.68 (d, J=7.6 Hz, 1H), 7.55-7.45 (m, 4H), 7.40-7.26 (m, 4H), 6.75 (s, 1H), 6.28 (s, 1H), 5.07-5.00 (m, 2H), 4.30-3.38 (m, 11H), 3.26-2.58 (m, 20H), 2.06-1.97 (m, 2H), 1.64-1.42 (m, 2H), 0.94 (d, J=6.8 Hz, 6H); LC-MS: m/z 1005.4 [M+H]⁺.

### Compound 137

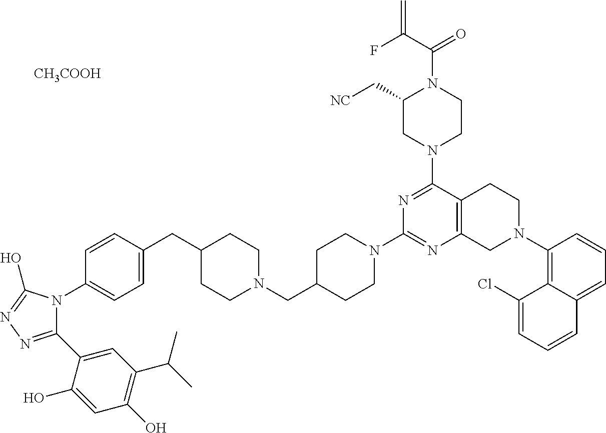 [SMILES: C=C(F)C(=O)N1CCN(c2nc(N3CCC(CN4CCC(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N ; "CH3COOH"]

### (S)-2-(4-(7-(8-chloronaphthalen-1-yl)-2-(4-((4-(4-(3-(2,4-dihydroxy-5-isopropylphenyl)-5-hydroxy-4H-1,2,4-triazol-4-yl)benzyl)piperidin-1-yl)methyl)piperidin-1-yl)-5,6,7,8-tetrahydropyrido[3,4-d]pyrimidin-4-yl)-1-(2-fluoroacryloyl)piperazin-2-yl)acetonitrile, acetic acid

¹H NMR (CD3OD, 400 MHz): δ 7.80 (d, J=8.0 Hz, 1H), 7.65-7.62 (m, 1H), 7.52-7.26 (m, 6H), 7.20 (d, J=8.0 Hz, 2H), 6.68 (s, 1H), 6.27 (s, 1H), 5.34-5.26 (m, 2H), 4.68 (d, J=10.8 Hz, 2H), 4.27-3.46 (m, 11H), 3.16-2.50 (m, 17H), 1.81-1.74 (m, 4H), 1.91 (s, 3H), 1.76-1.74 (m, 4H), 1.47-1.37 (m, 2H), 1.21-1.14 (m, 2H), 0.88 (d, J=6.8 Hz, 6H); LC-MS: m/z 994.5 [M-CH₃COOH+H]⁺.

### Compound 138

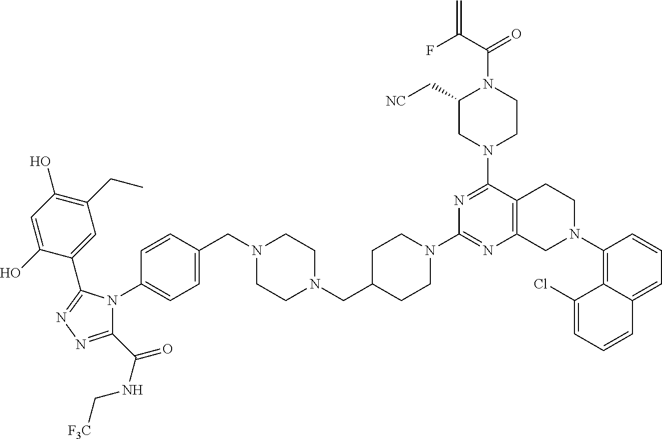 [SMILES: C=C(F)C(=O)N1CCN(c2nc(N3CCC(CN4CCN(Cc5ccc(-n6c(C(=O)NCC(F)(F)F)nnc6-c6cc(CC)c(O)cc6O)cc5)CC4)CC3)nc3c2CCN(c2cccc4cccc(Cl)c24)C3)C[C@@H]1CC#N]

### (S)-4-(4-((4-((1-(7-(8-chloronaphthalen-1-yl)-4-(3-(cyanomethyl)-4-(2-fluoroacryloyl)piperazin-1-yl)-5,6,7,8-tetrahydropyrido[3,4-d]pyrimidin-2-yl)piperidin-4-yl)methyl)piperazin-1-yl)methyl)phenyl)-5-(5-ethyl-2,4-dihydroxyphenyl)-N-(2,2,2-trifluoroethyl)-4H-1,2,4-triazole-3-carboxamide

¹H NMR (CD3OD, 400 MHz): δ 7.74 (d, J=8.4 Hz, 1H), 7.59-7.56 (m, 1H), 7.46-7.20 (m, 8H), 6.56 (s, 1H), 6.21 (s, 1H), 5.28-5.17 (m, 2H), 4.59 (d, J=12.8 Hz, 2H), 4.21-4.16 (m, 1H), 4.00-3.88 (m, 10H), 3.19-2.43 (m, 19H), 2.25-2.20 (m, 4H), 1.75-1.70 (m, 2H), 1.27-1.05 (m, 2H), 0.83 (t, J=7.2 Hz, 3H); LC-MS: m/z 1090.5 [M+H]⁺.

### Compound 139

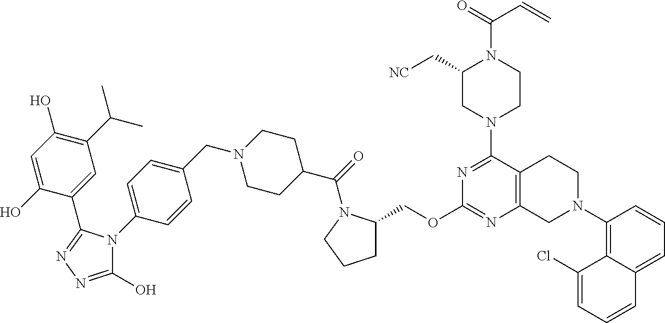 [SMILES: C=CC(=O)N1CCN(c2nc(OC[C@@H]3CCCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N]

### 2-((S)-1-acryloyl-4-(7-(8-chloronaphthalen-1-yl)-2-(((S)-1-(1-(4-(3-(2,4-dihydroxy-5-isopropylphenyl)-5-hydroxy-4H-1,2,4-triazol-4-yl)benzyl)piperidine-4-carbonyl)pyrrolidin-2-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.94 (s, 1H), 9.62 (d, J=11.2 Hz, 1H), 9.43 (d, J=10.4 Hz, 1H), 7.92-7.84 (m, 1H), 7.75-7.72 (m, 1H), 7.58-7.29 (m, 5H), 7.14-7.03 (m, 3H), 6.83 (brs, 1H), 6.73 (d, J=12.4 Hz, 2H), 6.27 (d, J=11.6 Hz, 1H), 6.18 (d, J=16.8 Hz, 1H), 5.77 (d, J=10.4 Hz, 1H), 4.96-4.75 (m, 1H), 4.30-3.69 (m, 8H), 3.51-3.48 (m, 3H), 3.18-2.79 (m, 10H), 2.63-2.57 (m, 1H), 2.41-2.35 (m, 1H), 2.03-1.82 (m, 6H), 1.63-1.46 (m, 5H), 0.91 (t, J=7.2 Hz, 6H). LC-MS: m/z 1006.4 [M+H]⁺.

### Compound 140

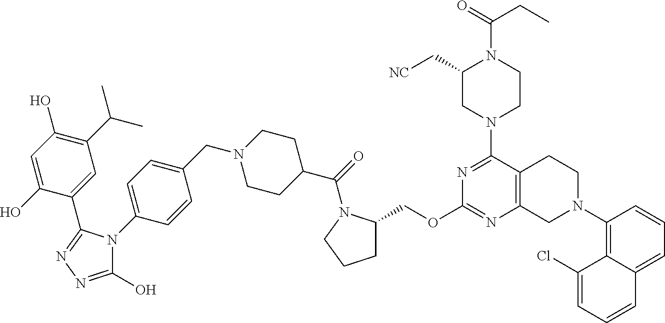 [SMILES: CCC(=O)N1CCN(c2nc(OC[C@@H]3CCCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N]

### 2-((S)-4-(7-(8-chloronaphthalen-1-yl)-2-(((S)-1-(1-(4-(3-(2,4-dihydroxy-5-isopropylphenyl)-5-hydroxy-4H-1,2,4-triazol-4-yl)benzyl)piperidine-4-carbonyl)pyrrolidin-2-yl)methoxy)-5,6,7,8-tetrahydropyrido[3,4-d]pyrimidin-4-yl)-1-propionylpiperazin-2-yl)acetonitrile

¹H NMR (DMSO-d₆, 400 MHz): δ 11.94 (s, 1H), 9.59 (d, J=12.4 Hz, 1H), 9.43 (d, J=10.4 Hz, 1H), 7.92-7.84 (m, 1H), 7.75-7.72 (m, 1H), 7.58-7.28 (m, 5H), 7.14-7.03 (m, 3H), 6.74 (d, J=10.4 Hz, 1H), 6.27 (d, J=12.4 Hz, 1H), 4.89-4.55 (m, 1H), 4.38-3.70 (m, 8H), 3.53-3.45 (m, 3H), 3.48-2.51 (m, 13H), 2.44-2.32 (m, 3H), 1.99-1.45 (m, 10H), 1.02 (brs, 3H), 0.91 (t, J=6.8 Hz, 6H). LC-MS: m/z 1008.5 [M+H]⁺.

### Compound 141

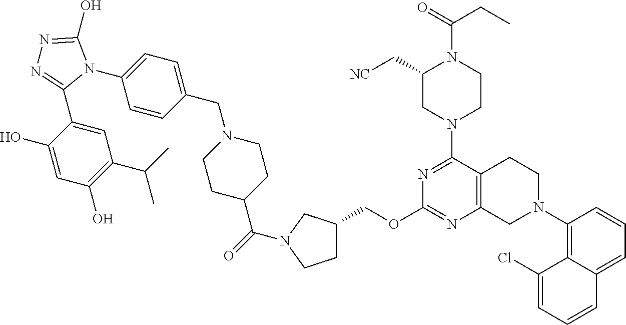 [SMILES: CCC(=O)N1CCN(c2nc(OC[C@@H]3CCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N]

### 2-((S)-4-(7-(8-chloronaphthalen-1-yl)-2-(((R)-1-(1-(4-(3-(2,4-dihydroxy-5-isopropylphenyl)-5-hydroxy-4H-1,2,4-triazol-4-yl)benzyl)piperidine-4-carbonyl)pyrrolidin-3-yl)methoxy)-5,6,7,8-tetrahydropyrido[3,4-d]pyrimidin-4-yl)-1-propionylpiperazin-2-yl)acetonitrile

¹H NMR (DMSO-d₆, 400 MHz): δ 11.92 (s, 1H), 9.59 (s, 1H), 9.41 (s, 1H), 7.92 (d, J=8.0 Hz, 1H), 7.74 (d, J=8.0 Hz, 1H), 7.58 (d, J=7.2 Hz, 1H), 7.53 (q, J=7.6 Hz, 1H), 7.45 (t, J=7.6 Hz, 1H), 7.33 (dd, J=16.0 Hz, J=8.0 Hz, 1H), 7.30 (d, J=7.2 Hz, 2H), 7.13 (d, J=7.2 Hz, 2H), 6.76 (s, 1H), 6.26 (s, 1H), 4.92-4.54 (m, 1H), 4.39-4.13 (m, 3H), 4.03-3.46 (m, 9H), 3.27-2.54 (m, 14H), 2.44-2.32 (m, 3H), 2.06-1.93 (m, 3H), 1.77-1.54 (m, 5H), 1.03-1.00 (m, 3H), 0.93 (dd, J=6.8 Hz, 2.4 Hz, 6H). LC-MS: m/z 1008.5 [M+H]⁺.

### Compound 142

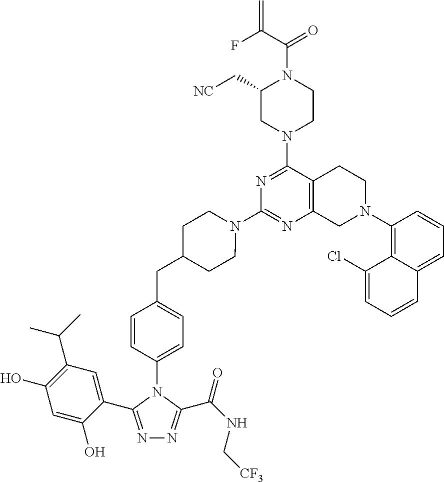 [SMILES: C=C(F)C(=O)N1CCN(c2nc(N3CCC(Cc4ccc(-n5c(C(=O)NCC(F)(F)F)nnc5-c5cc(C(C)C)c(O)cc5O)cc4)CC3)nc3c2CCN(c2cccc4cccc(Cl)c24)C3)C[C@@H]1CC#N]

### (S)-4-(4-((1-(7-(8-chloronaphthalen-1-yl)-4-(3-(cyanomethyl)-4-(2-fluoroacryloyl)piperazin-1-yl)-5,6,7,8-tetrahydropyrido[3,4-d]pyrimidin-2-yl)piperidin-4-yl)methyl)phenyl)-5-(2,4-dihydroxy-5-isopropylphenyl)-N-(2,2,2-trifluoroethyl)-4H-1,2,4-triazole-3-carboxamide

¹H NMR (CD3OD, 400 MHz): δ 7.71 (d, J=8.4 Hz, 1H), 7.56-7.54 (m, 1H), 7.44-7.35 (m, 2H), 7.28-7.16 (m, 6H), 6.56 (s, 1H), 6.23 (s, 1H), 5.26-5.14 (m, 2H), 4.59 (d, J=12.0 Hz, 1H), 4.20-4.15 (m, 1H), 3.92-3.85 (m, 4H). 3.61-3.38 (m, 3H), 3.09-2.53 (m, 14H), 1.93-1.92 (m, 1H), 1.78-1.74 (m, 1H), 1.64 (d, J=12.8 Hz, 2H), 1.16-1.08 (m, 2H), 0.75 (d, J=6.8 Hz, 6H); LC-MS: m/z 1006.4 [M+H]⁺.

### Compound 143

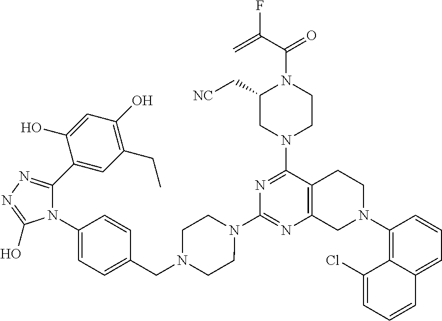 [SMILES: C=C(F)C(=O)N1CCN(c2nc(N3CCN(Cc4ccc(-n5c(O)nnc5-c5cc(CC)c(O)cc5O)cc4)CC3)nc3c2CCN(c2cccc4cccc(Cl)c24)C3)C[C@@H]1CC#N]

### (S)-2-(4-(7-(8-chloronaphthalen-1-yl)-2-(4-(4-(3-(5-ethyl-2,4-dihydroxyphenyl)-5-hydroxy-4H-1,2,4-triazol-4-yl)benzyl)piperazin-1-yl)-5,6,7,8-tetrahydropyrido[3,4-d]pyrimidin-4-yl)-1-(2-fluoroacryloyl)piperazin-2-yl)acetonitrile

¹H NMR (CD3OD, 400 MHz): δ 7.73 (d, J=7.6 Hz, 1H), 7.58 (d, J=8.4 Hz, 1H), 7.47-7.36 (m, 4H), 7.30-7.19 (m, 4H), 6.78 (s, 1H), 6.10 (s, 1H), 5.28-5.16 (m, 2H), 4.31-3.92 (m, 8H), 3.63-3.34 (m, 7H), 3.13-2.58 (m, 10H), 2.31 (q, J=7.6 Hz, 2H), 0.92 (t, J=7.6 Hz, 3H); LC-MS: m/z 884.3 [M+H]⁺.

### Compound 144

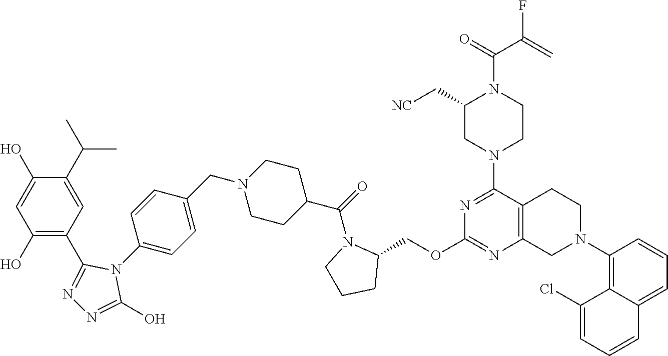 [SMILES: C=C(F)C(=O)N1CCN(c2nc(OC[C@@H]3CCCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N]

### 2-((S)-4-(7-(8-chloronaphthalen-1-yl)-2-(((S)-1-(1-(4-(3-(2,4-dihydroxy-5-isopropylphenyl)-5-hydroxy-4H-1,2,4-triazol-4-yl)benzyl)piperidine-4-carbonyl)pyrrolidin-2-yl)methoxy)-5,6,7,8-tetrahydropyrido[3,4-d]pyrimidin-4-yl)-1-(2-fluoroacryloyl)piperazin-2-yl)acetonitrile

¹H NMR (DMSO-d₆, 400 MHz): δ 11.93-11.92 (m, 1H), 9.61-9.58 (m, 1H), 9.43-9.41 (m, 1H), 7.91 (d, J=8.0 Hz, 1H), 7.73 (d, J=8.0 Hz, 1H), 7.58-7.27 (m, 5H), 7.13-7.00 (m, 3H), 6.75-6.73 (m, 1H), 6.29-6.26 (m, 1H), 5.38 (dd, J=17.6, 4.0 Hz, 1H), 5.33-5.20 (m, 1H), 4.87-4.64 (m, 1H), 4.31-3.71 (m, 8H), 3.50-3.43 (m, 3H), 3.32-2.32 (m, 11H), 2.01-1.83 (m, 6H), 1.63-1.47 (m, 5H), 0.93-0.85 (m, 6H). LC-MS: m/z 1024.3 [M+H]⁺.

### Compound 145

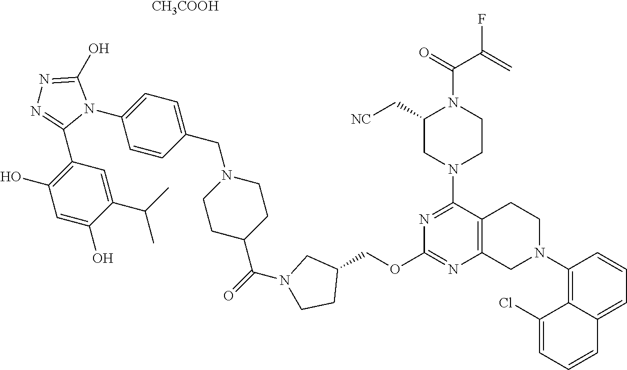 [SMILES: C=C(F)C(=O)N1CCN(c2nc(OC[C@@H]3CCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N ; "CH3COOH"]

### 2-((S)-4-(7-(8-chloronaphthalen-1-yl)-2-(((R)-1-(1-(4-(3-(2,4-dihydroxy-5-isopropylphenyl)-5-hydroxy-4H-1,2,4-triazol-4-yl)benzyl)piperidine-4-carbonyl)pyrrolidin-3-yl)methoxy)-5,6,7,8-tetrahydropyrido[3,4-d]pyrimidin-4-yl)-1-(2-fluoroacryloyl)piperazin-2-yl)acetonitrile, acetic acid

¹H NMR (DMSO-d₆, 400 MHz): δ 11.92 (s, 1H), 9.59 (s, 1H), 9.41 (s, 1H), 7.92 (d, J=8.0 Hz, 1H), 7.74 (d, J=8.0 Hz, 1H), 7.59-7.28 (m, 6H), 7.12 (d, J=8.0 Hz, 2H), 6.75 (s, 1H), 6.26 (s, 1H), 5.41-5.20 (m, 2H), 4.86-4.65 (m, 1H), 4.23-3.70 (m, 8H), 3.51-3.41 (m, 4H), 3.30-2.32 (m, 14H), 2.06-1.88 (m, 2H), 1.77-1.50 (m, 5H), 0.93 (d, J=7.2 Hz, 3H), 0.92 (d, J=7.2 Hz, 3H). LC-MS: m/z 1024.3 [M-CH₃COOH+H]⁺.

### Compound 146

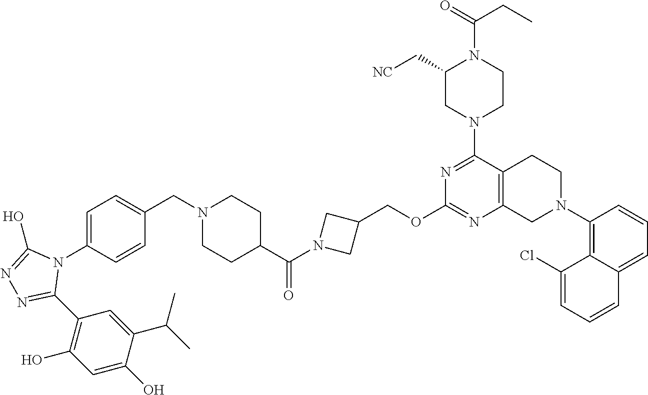 [SMILES: CCC(=O)N1CCN(c2nc(OCC3CN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N]

### (S)-2-(4-(7-(8-chloronaphthalen-1-yl)-2-((1-(1-(4-(3-(2,4-dihydroxy-5-isopropylphenyl)-5-hydroxy-4H-1,2,4-triazol-4-yl)benzyl)piperidine-4-carbonyl)azetidin-3-yl)methoxy)-5,6,7,8-tetrahydropyrido[3,4-d]pyrimidin-4-yl)-1-propionylpiperazin-2-yl)acetonitrile

¹H NMR (DMSO-d₆, 400 MHz): δ 11.93 (s, 1H), 9.59 (s, 1H), 9.42 (s, 1H), 7.92 (d, J=8.0 Hz, 1H), 7.74 (dd, J=8.0 Hz, J=3.2 Hz, 1H), 7.58 (d, J=8.0 Hz, 1H), 7.53 (q, J=8.0 Hz, 1H), 7.45 (t, J=8.0 Hz, 1H), 7.33 (dd, J=16.0 Hz, J=8.0 Hz, 1H), 7.28 (d, J=8.0 Hz, 2H), 7.12 (d, J=8.0 Hz, 2H), 6.75 (s, 1H), 6.26 (s, 1H), 4.89-4.55 (m, 1H), 4.34-3.41 (m, 13H), 3.26-2.67 (m, 12H), 2.55 (brs, 1H), 2.44-2.32 (m, 2H), 2.17-2.12 (m, 1H), 1.94-1.89 (m, 2H), 1.57-1.45 (m, 4H), 1.03-1.00 (m, 3H), 0.92 (d, J=7.2 Hz, 6H). LC-MS: m/z 994.5 [M+H]⁺.

### Compound 147

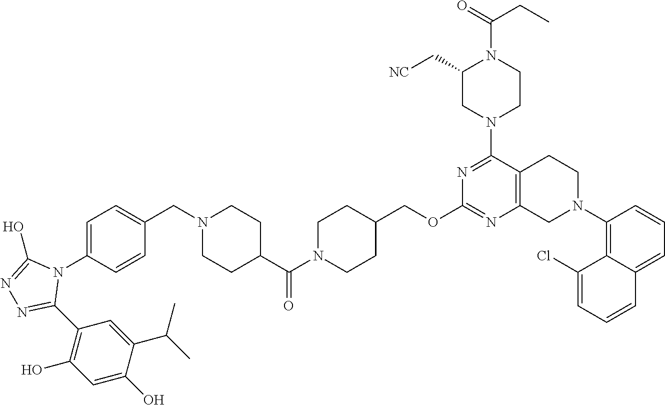 [SMILES: CCC(=O)N1CCN(c2nc(OCC3CCN(C(=O)C4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(4-(7-(8-chloronaphthalen-1-yl)-2-((1-(1-(4-(3-(2,4-dihydroxy-5-isopropylphenyl)-5-hydroxy-4H-1,2,4-triazol-4-yl)benzyl)piperidine-4-carbonyl)piperidin-4-yl)methoxy)-5,6,7,8-tetrahydropyrido[3,4-d]pyrimidin-4-yl)-1-propionylpiperazin-2-yl)acetonitrile

¹H NMR (DMSO-d₆, 400 MHz): δ 11.92 (s, 1H), 9.59 (s, 1H), 9.41 (s, 1H), 7.92 (d, J=8.0 Hz, 1H), 7.75-7.72 (m, 1H), 7.58 (d, J=7.2 Hz, 1H), 7.53 (q, J=8.0 Hz, 1H), 7.45 (t, J=8.0 Hz, 1H), 7.33 (dd, J=17.2 Hz, J=7.6 Hz, 1H), 7.29 (d, J=8.8 Hz, 2H), 7.12 (d, J=8.8 Hz, 2H), 6.76 (s, 1H), 6.26 (s, 1H), 4.89-4.56 (m, 1H), 4.42-3.43 (m, 11H), 3.26-2.64 (m, 12H), 2.55 (brs, 2H), 2.44-2.30 (m, 2H), 1.98 (brs, 3H), 1.79-1.54 (m, 6H), 1.03-1.00 (m, 3H), 0.93 (d, J=7.2 Hz, 6H). LC-MS: m/z 1023.4 [M+H]⁺.

### Compound 149

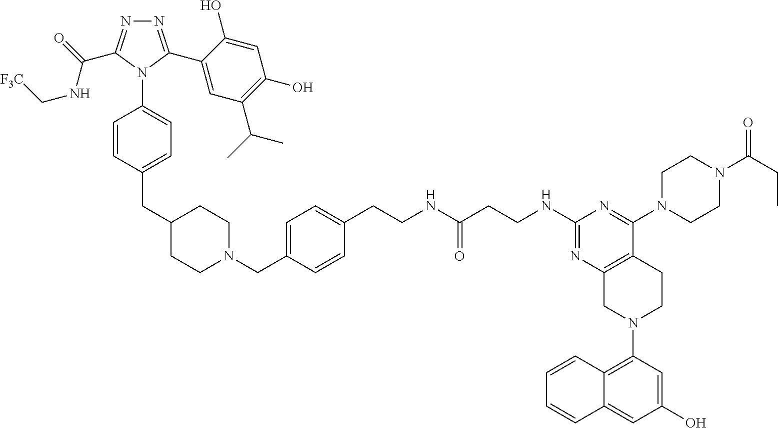 [SMILES: CCC(=O)N1CCN(c2nc(NCCC(=O)NCCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1]

### 4-(4-propionylpiperazin-1-yl)-5,6,7,8-tetrahydropyrido[3,4-d]pyrimidin-2-yl)amino)propanamido)ethyl)benzyl)piperidin-4-yl)methyl)phenyl)-N-(2,2,2-trifluoroethyl)-4H-1,2,4-triazole-3-carboxamide

¹H NMR (DMSO-d₆, 400 MHz): δ 10.54 (s, 1H), 9.76 (s, 1H), 9.63 (s, 1H), 9.57 (t, J=6.4 Hz, 1H), 8.00 (d, J=8.4 Hz, 1H), 7.90 (t, J=6.0 Hz, 1H), 7.65 (t, J=7.6 Hz, 1H), 7.37 (dd, J=7.6, 7.2 Hz, 1H), 7.27-7.10 (m, 9H), 6.84 (d, J=2.0 Hz, 1H), 6.75 (s, 1H), 6.59 (s, 1H), 6.45 (brs, 1H), 6.34 (s, 1H), 3.98-3.92 (m, 4H), 3.56-3.22 (m, 18H), 2.92-2.65 (m, 7H), 2.38-2.32 (m, 4H), 1.83-1.77 (m, 2H), 1.53-1.44 (m, 3H), 1.23-1.17 (m, 2H), 1.01 (t, J=7.2 Hz, 1H), 0.80 (d, J=6.8 Hz, 1H). LC-MS: m/z 1137.6 [M+H]⁺.

### Compound 150

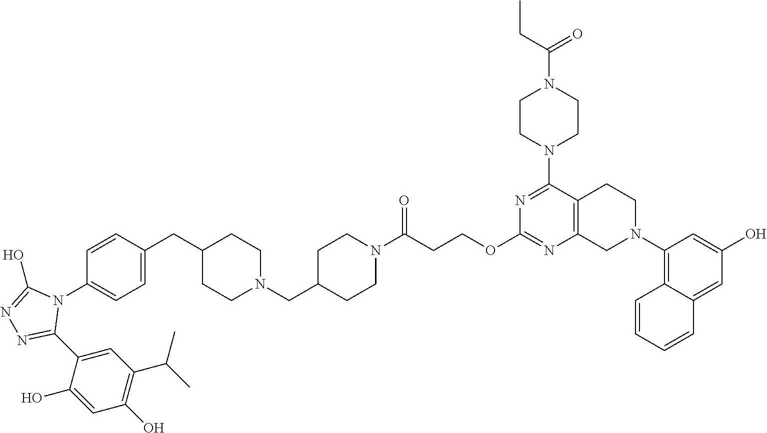 [SMILES: CCC(=O)N1CCN(c2nc(OCCC(=O)N3CCC(CN4CCC(Cc5ccc(-n6c(O)nnc6-c6cc(C(C)C)c(O)cc6O)cc5)CC4)CC3)nc3c2CCN(c2cc(O)cc4ccccc24)C3)CC1]

### 1-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-3-((7-(3-hydroxynaphthalen-1-yl)-4-(4-propionylpiperazin-1-yl)-5,6,7,8-tetrahydropyrido[3,4-d]pyrimidin-2-yl)oxy)propan-1-one

¹H NMR (DMSO-d₆, 400 MHz): δ 11.95 (s, 1H), 9.62 (brs, 1H), 9.38 (brs, 1H), 8.11 (t, J=4.4 Hz, 1H), 8.00 (d, J=8.0 Hz, 1H), 7.67 (d, J=8.0 Hz, 1H), 7.41-7.37 (m, 3H), 7.27 (t, J=7.2 Hz, 1H), 7.21 (d, J=7.2 Hz, 2H), 6.87 (d, J=1.6 Hz, 1H), 6.83 (s, 1H), 6.76 (d, J=1.6 Hz, 1H), 6.26 (s, 1H), 4.48 (t, J=6.0 Hz, 3H), 4.38 (d, J=13.2 Hz, 1H), 4.23 (d, J=3.6 Hz, 3H), 4.10 (s, 3H), 3.92 (d, J=12.4 Hz, 4H), 3.60 (s, 9H), 3.26 (brs, 2H), 3.04-3.53 (m, 12H), 2.37 (q, J=7.6 Hz, 2H), 1.96 (brs, 1H), 1.73 (d, J=12.0 Hz, 2H), 1.02 (t, J=7.2 Hz, 3H), 0.97 (d, J=7.2 Hz, 6H). LC-MS: m/z 994.5 [M+H]⁺.

### Compound 151

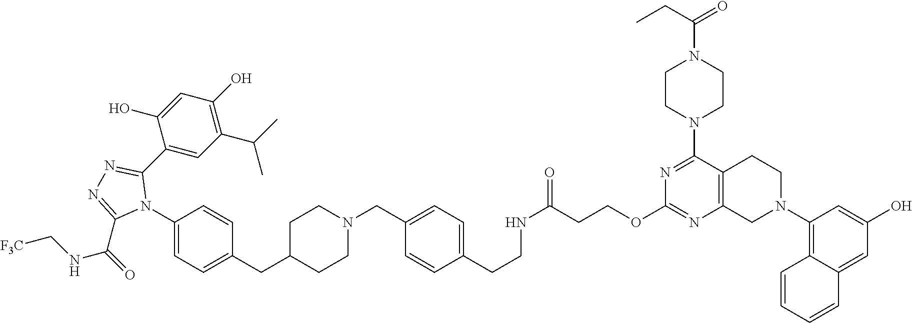 [SMILES: CCC(=O)N1CCN(c2nc(OCCC(=O)NCCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1]

### 5-(2,4-dihydroxy-5-isopropylphenyl)-4-(4-((1-(4-(2-(3-((7-(3-hydroxynaphthalen-1-yl)-4-(4-propionylpiperazin-1-yl)-5,6,7,8-tetrahydropyrido[3,4-d]pyrimidin-2-yl)oxy)propanamido)ethyl)benzyl)piperidin-4-yl)methyl)phenyl)-N-(2,2,2-trifluoroethyl)-4H-1,2,4-triazole-3-carboxamide

¹H NMR (DMSO-d₆, 400 MHz): δ 9.79 (brs, 1H), 9.60 (t, J=4.8 Hz, 1H), 9.35 (brs, 1H), 8.11 (t, J=4.4 Hz, 1H), 8.00 (d, J=8.0 Hz, 1H), 7.67 (d, J=8.0 Hz, 1H), 7.46-7.24 (m, 10H), 6.87 (d, J=1.6 Hz, 1H), 6.77 (d, J=2.4 Hz, 1H), 6.60 (s, 1H), 6.34 (s, 1H), 4.46 (t, J=6.0 Hz, 3H), 4.36 (s, 1H), 4.23 (d, J=3.6 Hz, 3H), 4.10 (s, 3H), 4.00-3.91 (m, 4H), 3.60 (s, 8H), 3.35-3.26 (m, 6H), 2.94-2.73 (m, 7H), 2.56-2.53 (m, 3H), 2.37 (q, J=7.2 Hz, 2H), 1.76-1.71 (m, 3H), 1.39 (dd, J=16.0 Hz, J=8.8 Hz, 2H), 1.01 (t, J=7.2 Hz, 3H), 0.82 (d, J=7.2 Hz, 6H). LC-MS: m/z 1138.5 [M+H]⁺.

### Compound 152

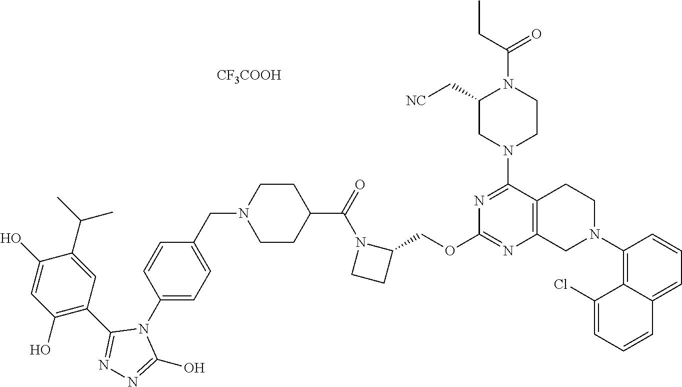 [SMILES: CCC(=O)N1CCN(c2nc(OC[C@@H]3CCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N ; "CF3COOH"]

### 2-((S)-4-(7-(8-chloronaphthalen-1-yl)-2-(((S)-1-(1-(4-(3-(2,4-dihydroxy-5-isopropylphenyl)-5-hydroxy-4H-1,2,4-triazol-4-yl)benzyl)piperidine-4-carbonyl)azetidin-2-yl)methoxy)-5,6,7,8-tetrahydropyrido[3,4-d]pyrimidin-4-yl)-1-propionylpiperazin-2-yl)acetonitrile, trifluoroacetic acid

¹H NMR (DMSO-d₆, 400 MHz): δ 11.98 (s, 1H), 9.62-9.61 (m, 1H), 9.36-9.33 (m, 1H), 8.72 (brs, 2H), 7.93-7.87 (m, 1H), 7.75-7.72 (m, 1H), 7.57-7.25 (m, 7H), 7.11-6.73 (m, 2H), 6.28-6.24 (m, 1H), 4.88-4.79 (m, 1H), 4.58-3.74 (m, 15H), 3.07-2.67 (m, 11H), 2.41-1.68 (m, 11H), 2.63-2.57 (m, 1H), 2.41-2.35 (m, 1H), 2.03-1.82 (m, 6H), 1.52 (d, J=6.8 Hz, 3H), 1.02-1.0.98 (m, 6H). LC-MS: m/z 994.4 [M-CF₃COOH+H]⁺.

### Compound 153

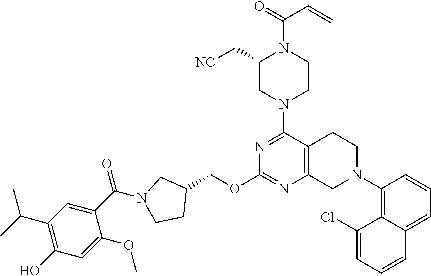

### 2-((S)-1-acryloyl-4-(7-(8-chloronaphthalen-1-yl)-2-(((R)-1-(4-hydroxy-5-isopropyl-2-methoxybenzoyl)pyrrolidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 9.62 (brs, 1H), 7.92 (d, J=8.0 Hz, 1H), 7.74 (d, J=8.0 Hz, 1H), 7.59-7.50 (m, 2H), 7.45 (t, J=8.0 Hz, 1H), 7.37-7.30 (m, 1H), 6.88 (d, J=11.2 Hz, 1H), 6.84 (brs, 1H), 6.45 (s, 1H), 6.18 (d, J=16.8 Hz, 1H), 5.78 (d, J=10.4 Hz, 1H), 4.95-4.75 (m, 1H), 4.32-3.72 (m, 6H), 3.67 (d, J=11.2 Hz, 3H), 3.58-3.36 (m, 3H), 3.25-2.83 (m, 11H), 2.68-2.55 (m, 2H), 2.07-1.93 (m, 1H), 1.73-1.66 (m, 1H), 1.11-1.03 (m, 6H). LC-MS: m/z 764.3 [M+H]⁺.

### Compound 154

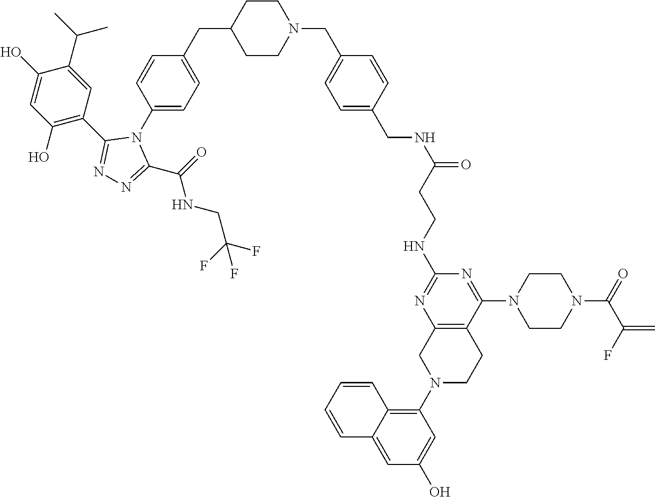 [SMILES: C=C(F)C(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1]

### 5-(2,4-dihydroxy-5-isopropylphenyl)-4-(4-((1-(4-((3-((4-(4-(2-fluoroacryloyl)piperazin-1-yl)-7-(3-hydroxynaphthalen-1-yl)-5,6,7,8-tetrahydropyrido[3,4-d]pyrimidin-2-yl)amino)propanamido)methyl)benzyl)piperidin-4-yl)methyl)phenyl)-N-(2,2,2-trifluoroethyl)-4H-1,2,4-triazole-3-carboxamide

¹H NMR (DMSO-d₆, 400 MHz): δ 10.54 (s, 1H), 9.76 (s, 1H), 9.63 (s, 1H), 9.57 (t, J=6.4 Hz, 1H), 8.30 (t, J=6.4 Hz, 1H), 8.00 (d, J=8.4 Hz, 1H), 7.65 (d, J=7.6 Hz, 1H), 7.37 (t, J=7.2 Hz, 1H), 7.27-7.14 (m, 10H), 6.84 (s, 1H), 6.75 (s, 1H), 6.59 (s, 2H), 6.34 (s, 1H), 5.34-5.29 (m, 2H), 5.16 (d, J=4 Hz, 1H), 4.24 (d, J=5.6 Hz, 2H), 3.98-3.93 (m, 4H), 3.66 (s, 4H) 3.49-3.33 (m, 6H), 3.28-3.24 (m, 3H), 2.89-2.72 (m, 6H), 2.51-2.42 (m, 5H), 1.84-1.75 (m, 2H), 1.54-1.43 (m, 2H), 0.80 (d, J=6.8 Hz, 6H). LC-MS: m/z 1139.5 [M+H]⁺.

### Compound 155

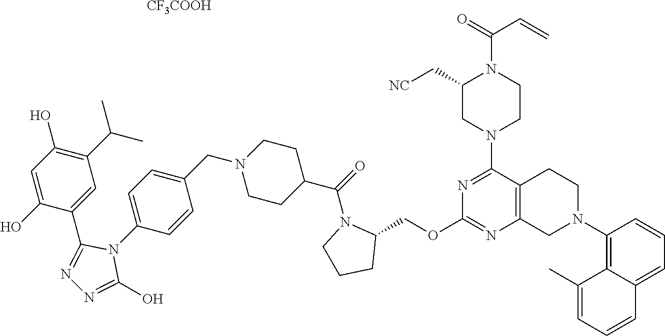 [SMILES: C=CC(=O)N1CCN(c2nc(OC[C@@H]3CCCN3C(=O)C3CCN(Cc4ccc(-n5c(O)nnc5-c5cc(C(C)C)c(O)cc5O)cc4)CC3)nc3c2CCN(c2cccc4cccc(C)c24)C3)C[C@@H]1CC#N ; "CF3COOH"]

### 2-((S)-1-acryloyl-4-(2-(((S)-1-(1-(4-(3-(2,4-dihydroxy-5-isopropylphenyl)-5-hydroxy-4H-1,2,4-triazol-4-yl)benzyl)piperidine-4-carbonyl)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile, trifluoroacetic acid

¹H NMR (DMSO-d₆, 400 MHz): δ 11.99-11.97 (m, 1H), 9.60 (brs, 1H), 9.52 (brs, 1H), 9.33 (brs, 1H), 7.76-7.66 (m, 2H), 7.49-7.25 (m, 7H), 7.14 (d, J=8.0 Hz, 1H), 6.86-6.75 (m, 2H), 6.27-6.17 (m, 2H), 5.77 (d, J=10.0 Hz, 1H), 4.96-4.75 (m, 1H), 4.44-3.93 (m, 9H), 3.36-2.90 (m, 14H), 2.85 (s, 3H), 2.70-2.67 (m, 2H), 2.02-1.75 (m, 6H), 0.99 (d, J=6.8 Hz, 6H). LC-MS: m/z 986.5 [M-CF₃COOH+H]⁺.

### Compound 156

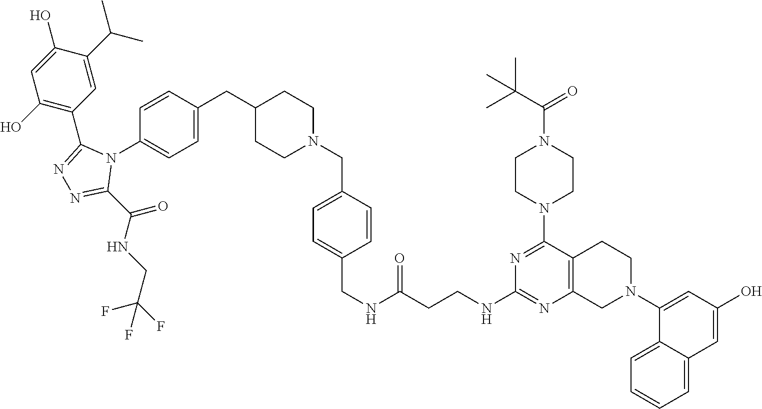 [SMILES: CC(C)c1cc(-c2nnc(C(=O)NCC(F)(F)F)n2-c2ccc(CC3CCN(Cc4ccc(CNC(=O)CCNc5nc6c(c(N7CCN(C(=O)C(C)(C)C)CC7)n5)CCN(c5cc(O)cc7ccccc57)C6)cc4)CC3)cc2)c(O)cc1O]

### (4-pivaloylpiperazin-1-yl)-5,6,7,8-tetrahydropyrido[3,4-d]pyrimidin-2-yl)amino)propanamido)methyl)benzyl)piperidin-4-yl)methyl)phenyl)-N-(2,2,2-trifluoroethyl)-4H-1,2,4-triazole-3-carboxamide

¹H NMR (DMSO-d₆, 400 MHz): δ 10.54 (s, 1H), 9.80 (s, 2H), 8.54 (s, 1H), 7.99 (d, J=8.4 Hz, 1H), 7.77-7.67 (m, 1H), 7.46-7.24 (m, 10H), 6.89 (s, 1H), 6.75 (s, 2H), 6.60 (s, 2H), 6.34 (s, 2H), 4.44-3.57 (m, 20H), 2.87-2.64 (m, 6H), 1.47-1.21 (m, 13H), 0.80 (d, J=6.8 Hz, 6H). LC-MS: m/z 1152.6 [M+H]⁺.

### Compound 157

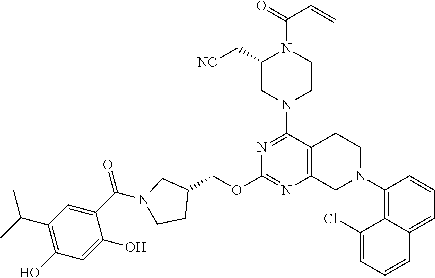

### 2-((S)-1-acryloyl-4-(7-(8-chloronaphthalen-1-yl)-2-(((R)-1-(2,4-dihydroxy-5-isopropylbenzoyl)pyrrolidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 10.50 (brs, 1H), 9.62 (brs, 1H), 7.92 (d, J=8.0 Hz, 1H), 7.75 (d, J=8.0 Hz, 1H), 7.60-7.51 (m, 2H), 7.45 (t, J=8.0 Hz, 1H), 7.34 (dd, J=13.2 Hz, 7.2 Hz, 1H), 7.05 (s, 1H), 6.83 (brs, 1H), 6.32 (s, 1H), 6.18 (d, J=16.8 Hz, 1H), 5.78 (d, J=14.4 Hz, 1H), 4.93-4.75 (m, 1H), 4.41-3.72 (m, 9H), 3.17-3.03 (m, 9H), 2.71-2.63 (m, 2H), 2.06-1.97 (m, 2H), 1.75-1.71 (m, 1H), 1.10 (J=7.2 Hz, 6H). LC-MS: m/z 750.3 [M+H]⁺.

### Compound 158

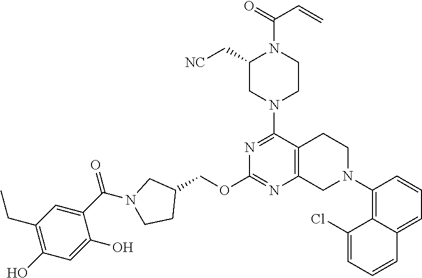

### 2-((S)-1-acryloyl-4-(7-(8-chloronaphthalen-1-yl)-2-(((R)-1-(5-ethyl-2,4-dihydroxybenzoyl)pyrrolidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 10.56 (brs, 1H), 9.66 (brs, 1H), 7.92 (d, J=8.0 Hz, 1H), 7.74 (dd, J=8.0 Hz, 4.0 Hz, 1H), 7.58 (d, J=7.2 Hz, 1H), 7.53 (q, J=7.2 Hz, 1H), 7.45 (t, J=8.0 Hz, 1H), 7.33 (dd, J=16.4 Hz, 7.6 Hz, 1H), 7.03 (s, 1H), 6.84 (brs, 1H), 6.32 (s, 1H), 6.18 (d, J=16.8 Hz, 1H), 5.77 (d, J=10.8 Hz, 1H), 4.95-4.74 (m, 1H), 4.41-3.48 (m, 13H), 3.18-2.61 (m, 6H), 2.41 (dd, J=14.8 Hz, 7.2 Hz, 2H), 2.03-1.97 (m, 2H), 1.73-1.70 (m, 1H), 1.07-1.03 (m, 3H). LC-MS: m/z 736.4 [M+H]⁺.

### Compound 159

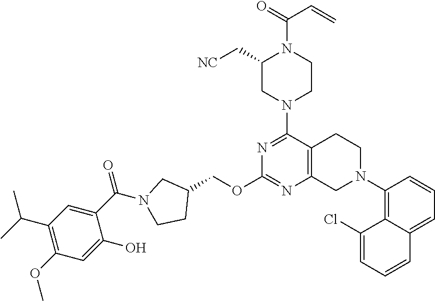

### 2-((S)-1-acryloyl-4-(7-(8-chloronaphthalen-1-yl)-2-(((R)-1-(2-hydroxy-5-isopropyl-4-methoxybenzoyl)pyrrolidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.36 (brs, 1H), 7.92 (d, J=8.0 Hz, 1H), 7.74 (dd, J=8.0 Hz, 4.0 Hz, 1H), 7.58 (d, J=8.0 Hz, 1H), 7.53 (q, J=7.6 Hz, 1H), 7.45 (t, J=8.0 Hz, 1H), 7.33 (dd, J=15.6 Hz, 7.6 Hz, 1H), 7.09 (s, 1H), 6.84 (brs, 1H), 6.44 (d, J=0.8 Hz, 1H), 6.18 (d, J=17.2 Hz, 1H), 5.77 (d, J=12.0 Hz, 1H), 4.96-4.74 (m, 1H), 4.38-3.88 (m, 7H), 3.76 (s, 1H), 3.62-3.49 (m, 5H), 3.12-2.64 (m, 8H), 2.04-1.95 (m, 2H), 1.76-1.70 (m, 1H), 0.99 (d, J=6.8 Hz, 6H). LC-MS: m/z 764.3 [M+H]⁺.

### Compound 160

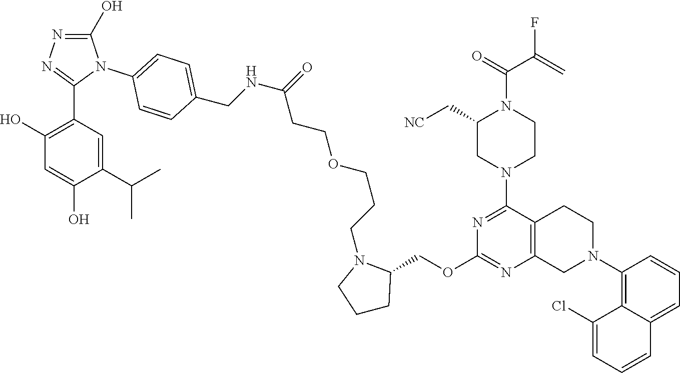 [SMILES: C=C(F)C(=O)N1CCN(c2nc(OC[C@@H]3CCCN3CCCOCCC(=O)NCc3ccc(-n4c(O)nnc4-c4cc(C(C)C)c(O)cc4O)cc3)nc3c2CCN(c2cccc4cccc(Cl)c24)C3)C[C@@H]1CC#N]

### 3-(3-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)propoxy)-N-(4-(3-(2,4-dihydroxy-5-isopropylphenyl)-5-hydroxy-4H-1,2,4-triazol-4-yl)benzyl)propenamide

¹H NMR (DMSO-d₆, 400 MHz): δ 11.87 (s, 1H), 9.54 (s, 1H), 9.35 (s, 1H), 8.31 (brs, 1H), 7.91 (d, J=7.6 Hz, 1H), 7.73 (dd, J=8.0, 2.8 Hz, 1H), 7.58-7.42 (m, 2H), 7.33 (dd, J=14.8, 7.2 Hz, 1H), 7.18 (d, J=8.4 Hz, 2H), 7.08 (d, J=8.4 Hz, 2H), 6.85 (s, 1H), 6.23 (s, 1H), 5.41-5.20 (m, 2H), 4.85-4.58 (m, 1H), 4.21-4.14 (m, 4H), 4.03-3.70 (m, 5H), 3.54-3.48 (m, 3H), 3.37-3.31 (m, 4H), 3.23-2.67 (m, 12H), 2.33-2.11 (m, 4H), 1.90-1.63 (m, 6H), 0.99 (d, J=7.2 Hz, 6H). LC-MS: m/z 1042.5 [M+H]⁺.

### Compound 161

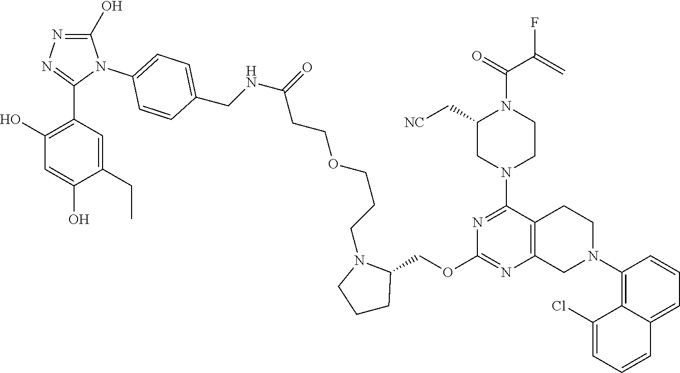 [SMILES: C=C(F)C(=O)N1CCN(c2nc(OC[C@@H]3CCCN3CCCOCCC(=O)NCc3ccc(-n4c(O)nnc4-c4cc(CC)c(O)cc4O)cc3)nc3c2CCN(c2cccc4cccc(Cl)c24)C3)C[C@@H]1CC#N]

### 3-(3-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)propoxy)-N-(4-(3-(5-ethyl-2,4-dihydroxyphenyl)-5-hydroxy-4H-1,2,4-triazol-4-yl)benzyl)propanamide

¹H NMR (DMSO-d₆, 400 MHz): δ 11.85 (s, 1H), 9.51 (s, 1H), 9.33 (s, 1H), 8.30 (t, J=5.2 Hz, 1H), 7.91 (d, J=8.4 Hz, 1H), 7.73 (dd, J=8.0, 3.2 Hz, 1H), 7.58-7.42 (m, 2H), 7.33 (dd, J=15.2, 7.6 Hz, 1H), 7.17 (d, J=10.4 Hz, 1H), 7.07 (d, J=8.4 Hz, 1H), 6.87 (s, 1H), 6.22 (s, 1H), 5.40-5.20 (m, 2H), 4.83-4.64 (m, 1H), 4.21-4.14 (m, 4H), 4.03-3.70 (m, 4H), 3.53-3.47 (m, 3H), 3.36-2.71 (m, 13H), 2.38-2.30 (m, 4H), 2.17-1.83 (m, 3H), 1.70-1.58 (m, 6H), 0.99 (t, J=7.2 Hz, 3H). LC-MS: m/z 1028.6 [M+H]⁺.

### Compound 162

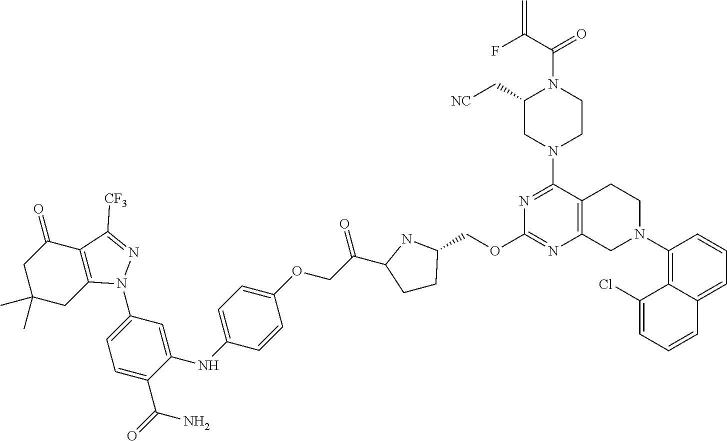 [SMILES: C=C(F)C(=O)N1CCN(c2nc(OC[C@@H]3CCC(C(=O)COc4ccc(Nc5cc(-n6nc(C(F)(F)F)c7c6CC(C)(C)CC7=O)ccc5C(N)=O)cc4)N3)nc3c2CCN(c2cccc4cccc(Cl)c24)C3)C[C@@H]1CC#N]

### 2-((4-(2-((R)-3-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)-2-oxoethoxy)phenyl)amino)-4-(6,6-dimethyl-4-oxo-3-(trifluoromethyl)-4,5,6,7-tetrahydro-1H-indazol-1-yl)benzamide

¹H NMR (DMSO-d₆, 400 MHz): δ 10.09 (s, 1H), 8.18 (s, 1H), 7.92-7.90 (m, 2H), 7.73 (d, J=8.0 Hz, 1H), 7.58-7.50 (m, 3H), 7.43 (dt, J=8.0 Hz, 3.2 Hz, 1H), 7.36-7.30 (m, 1H), 7.22-7.18 (m, 2H), 7.02 (s, 1H), 6.95-6.92 (m, 3H), 5.38 (dd, J=18.0 Hz, 4.4 Hz, 1H), 5.27 (d, J=50.0 Hz, 1H), 4.84-4.71 (m, 1H), 4.70 (s, 2H), 4.27-3.48 (m, 13H), 3.26-2.89 (m, 10H), 3.27-2.55 (m, 3H), 2.40 (d, J=5.6 Hz, 2H), 2.12-1.62 (m, 3H), 1.02 (d, J=2.4 Hz, 6H). LC-MS: m/z 1088.4 [M+H]⁺.

### Compound 163

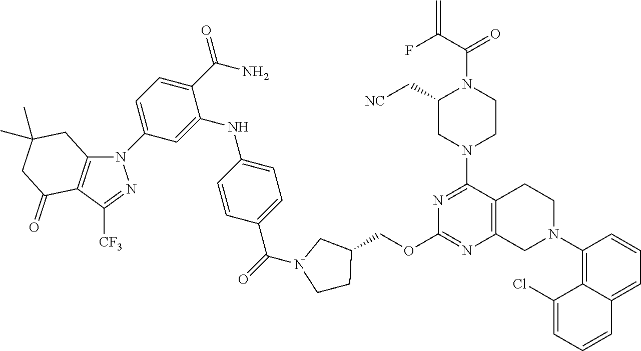 [SMILES: C=C(F)C(=O)N1CCN(c2nc(OC[C@@H]3CCN(C(=O)c4ccc(Nc5cc(-n6nc(C(F)(F)F)c7c6CC(C)(C)CC7=O)ccc5C(N)=O)cc4)C3)nc3c2CCN(c2cccc4cccc(Cl)c24)C3)C[C@@H]1CC#N]

### 2-((4-((R)-3-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidine-1-carbonyl)phenyl)amino)-4-(6,6-dimethyl-4-oxo-3-(trifluoromethyl)-4,5,6,7-tetrahydro-1H-indazol-1-yl)benzamide ¹H NMR (DMSO-d₆, 400 MHz): δ 10.33 (s, 1H), 8.26 (s, 1H), 7.94-7.90 (m, 2H), 7.75-7.72 (m, 2H), 7.57-7.27 (m, 9H), 7.14 (dd, J=12.8 Hz, 8.8 Hz, 1H), 5.38 (d, J=16.0 Hz, 1H), 5.27 (d, J=48.8 Hz, 1H), 4.84-4.71 (m, 1H), 4.27-3.38 (m, 12H), 3.30 (s, 2H), 3.10-2.63 (m, 7H), 2.42-2.32 (m, 4H), 2.04 (brs, 1H), 1.73 (brs, 1H), 1.02 (s, 6H). LC-MS: m/z 1058.4 [M+H]⁺

### Compound 164

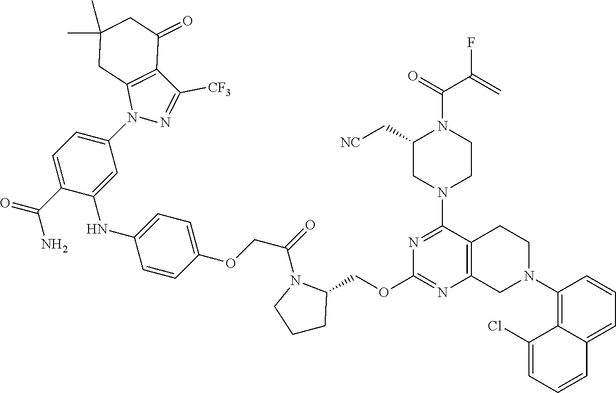 [SMILES: C=C(F)C(=O)N1CCN(c2nc(OC[C@@H]3CCCN3C(=O)COc3ccc(Nc4cc(-n5nc(C(F)(F)F)c6c5CC(C)(C)CC6=O)ccc4C(N)=O)cc3)nc3c2CCN(c2cccc4cccc(Cl)c24)C3)C[C@@H]1CC#N]

### 2-((4-(2-((S)-2-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)-2-oxoethoxy)phenyl)amino)-4-(6,6-dimethyl-4-oxo-3-(trifluoromethyl)-4,5,6,7-tetrahydro-1H-indazol-1-yl)benzamide

¹H NMR (DMSO-d₆, 400 MHz): δ 10.08 (brs, 1H), 8.20 (brs, 1H), 7.93-7.84 (m, 2H), 7.73-7.67 (m, 1H), 7.59 (brs, 1H), 7.56-7.25 (m, 4H), 7.17-6.81 (m, 6H), 5.39-5.19 (m, 2H), 5.00-4.70 (m, 3H), 4.36-3.62 (m, 10H), 3.28-2.65 (m, 10H), 2.40-2.34 (m, 2H), 2.05-1.91 (m, 4H), 1.02-0.96 (m, 6H). LC-MS: m/z 1088.4 [M+H]⁺.

### Compound 165

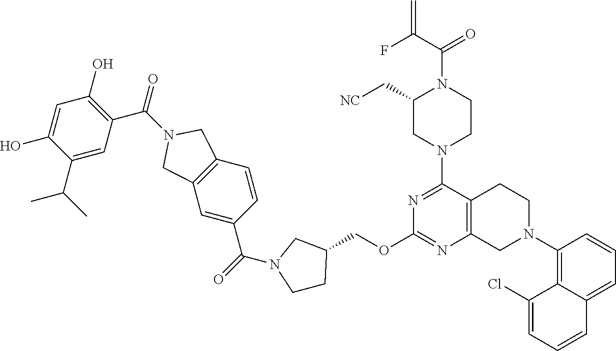 [SMILES: C=C(F)C(=O)N1CCN(c2nc(OC[C@@H]3CCN(C(=O)c4ccc5c(c4)CN(C(=O)c4cc(C(C)C)c(O)cc4O)C5)C3)nc3c2CCN(c2cccc4cccc(Cl)c24)C3)C[C@@H]1CC#N]

### 2-((S)-4-(7-(8-chloronaphthalen-1-yl)-2-(((R)-1-(2-(2,4-dihydroxy-5-isopropylbenzoyl)isoindoline-5-carbonyl)pyrrolidin-3-yl)methoxy)-5,6,7,8-tetrahydropyrido[3,4-d]pyrimidin-4-yl)-1-(2-fluoroacryloyl)piperazin-2-yl)acetonitrile

¹H NMR (DMSO-d₆, 400 MHz): δ 10.06 (brs, 1H), 9.66 (brs, 1H), 7.92 (d, J=8.0 Hz, 1H), 7.74 (d, J=8.0 Hz, 1H), 7.59-7.29 (m, 7H), 7.02 (s, 1H), 6.40 (s, 1H), 5.39 (d, J=18.0 Hz, 1H), 5.27 (d, J=50.4 Hz, 1H), 4.78 (brs, 4H), 4.26-3.47 (m, 12H), 3.22-2.86 (m, 8H), 2.71-2.54 (m, 1H), 2.06-1.73 (m, 3H), 1.14-1.11 (m, 6H). LC-MS: m/z 913.4 [M+H]⁺.

### Compound 166

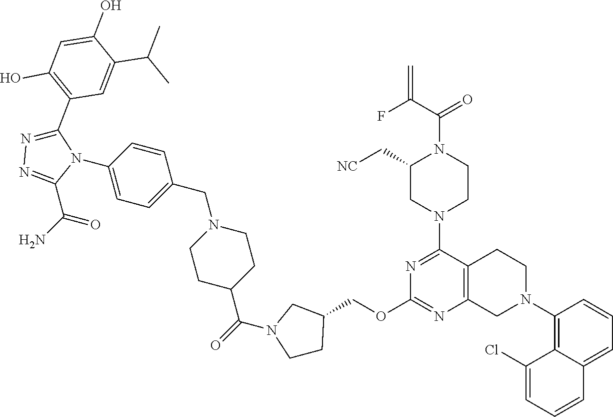 [SMILES: C=C(F)C(=O)N1CCN(c2nc(OC[C@@H]3CCN(C(=O)C4CCN(Cc5ccc(-n6c(C(N)=O)nnc6-c6cc(C(C)C)c(O)cc6O)cc5)CC4)C3)nc3c2CCN(c2cccc4cccc(Cl)c24)C3)C[C@@H]1CC#N]

### 4-(4-((4-((R)-3-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidine-1-carbonyl)piperidin-1-yl)methyl)phenyl)-5-(2,4-dihydroxy-5-isopropylphenyl)-4H-1,2,4-triazole-3-carboxamide

¹H NMR (DMSO-d₆, 400 MHz): δ 10.65 (brs, 1H), 9.81 (brs, 1H), 8.26 (s, 1H), 7.92 (d, J=8.0 Hz, 1H), 7.74 (d, J=8.0 Hz, 1H), 7.73 (s, 1H), 7.58 (d, J=6.8 Hz, 1H), 7.53 (dd, J=13.6 Hz, 7.6 Hz, 1H), 7.45 (t, J=7.6 Hz, 1H), 7.38-7.28 (m, 5H), 6.56 (d, J=2.0 Hz, 1H), 6.35 (s, 1H), 5.39 (dd, J=18.4 Hz, 3.6 Hz, 1H), 5.27 (d, J=46.4 Hz, 1H), 4.86 (brs, 1H), 4.27-3.46 (m, 15H), 3.14-2.67 (m, 10H), 2.38-2.31 (m, 2H), 2.08-1.95 (m, 4H), 1.79-1.56 (m, 5H), 0.79 (dd, J=6.8 Hz, 3.6 Hz, 6H). LC-MS: m/z 1051.4 [M+H]⁺.

### Compound 167

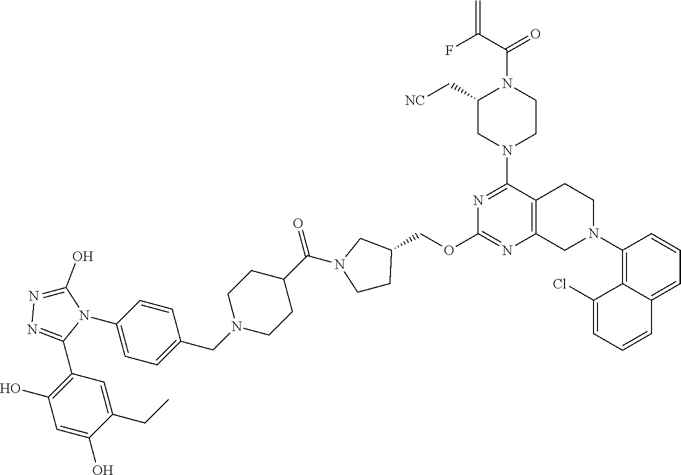 [SMILES: C=C(F)C(=O)N1CCN(c2nc(OC[C@@H]3CCN(C(=O)C4CCN(Cc5ccc(-n6c(O)nnc6-c6cc(CC)c(O)cc6O)cc5)CC4)C3)nc3c2CCN(c2cccc4cccc(Cl)c24)C3)C[C@@H]1CC#N]

### 2-((S)-4-(7-(8-chloronaphthalen-1-yl)-2-(((R)-1-(1-(4-(3-(5-ethyl-2,4-dihydroxyphenyl)-5-hydroxy-4H-1,2,4-triazol-4-yl)benzyl)piperidine-4-carbonyl)pyrrolidin-3-yl)methoxy)-5,6,7,8-tetrahydropyrido[3,4-d]pyrimidin-4-yl)-1-(2-fluoroacryloyl)piperazin-2-yl)acetonitrile

¹H NMR (DMSO-d₆, 400 MHz): δ 11.90 (s, 1H), 9.56 (s, 1H), 9.37 (s, 1H), 9.57 (t, J=6.4 Hz, 1H), 7.92 (d, J=8.4 Hz, 1H), 7.74 (d, J=8.0 Hz, 1H), 7.58-7.32 (m, 6H), 7.11 (d, J=7.6 Hz, 1H), 6.81 (s, 1H), 6.25 (s, 1H), 5.17-5.49 (m, 2H), 4.21-3.44 (m, 14H), 3.33-2.52 (m, 10H), 2.35-2.32 (m, 3H), 2.12-1.48 (m, 10H), 0.95 (t, J=7.6 Hz, 3H). LC-MS: m/z 1010.4 [M+H]⁺.

### Compound 168

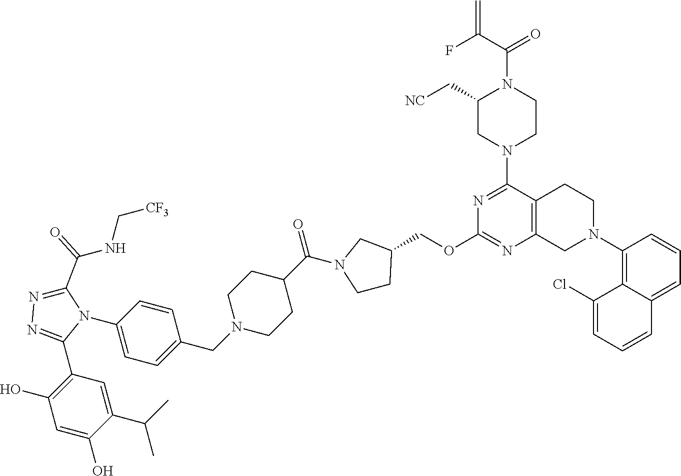 [SMILES: C=C(F)C(=O)N1CCN(c2nc(OC[C@@H]3CCN(C(=O)C4CCN(Cc5ccc(-n6c(C(=O)NCC(F)(F)F)nnc6-c6cc(C(C)C)c(O)cc6O)cc5)CC4)C3)nc3c2CCN(c2cccc4cccc(Cl)c24)C3)C[C@@H]1CC#N]

### 4-(4-((4-((R)-3-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidine-1-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide

¹H NMR (DMSO-d₆, 400 MHz): δ 10.51 (s, 1H), 9.59 (t, J=6.4 Hz, 1H), 7.92 (d, J=8.4 Hz, 1H), 7.74 (d, J=8.0 Hz, 1H), 7.59-7.30 (m, 9H), 6.61 (s, 1H), 6.35 (s, 1H), 5.41-5.37 (m, 2H), 4.21-3.36 (m, 19H), 3.25-3.16 (m, 5H), 1.99-1.59 (m, 12H), 1.23 (s, 2H), 0.82-0.80 (m, 6H). LC-MS: m/z 1133.4 [M+H]⁺.

### Compound 169

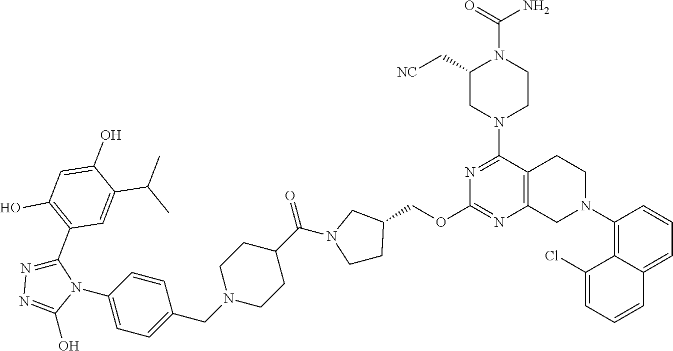 [SMILES: CC(C)c1cc(-c2nnc(O)n2-c2ccc(CN3CCC(C(=O)N4CC[C@@H](COc5nc6c(c(N7CCN(C(N)=O)[C@@H](CC#N)C7)n5)CCN(c5cccc7cccc(Cl)c57)C6)C4)CC3)cc2)c(O)cc1O]

### (S)-4-(7-(8-chloronaphthalen-1-yl)-2-(((R)-1-(1-(4-(3-(2,4-dihydroxy-5-isopropylphenyl)-5-hydroxy-4H-1,2,4-triazol-4-yl)benzyl)piperidine-4-carbonyl)pyrrolidin-3-yl)methoxy)-5,6,7,8-tetrahydropyrido[3,4-d]pyrimidin-4-yl)-2-(cyanomethyl)piperazine-1-carboxamide

¹H NMR (DMSO-d₆, 400 MHz): δ 11.92 (s, 1H), 9.58 (s, 1H), 9.40 (s, 1H), 7.91 (d, J=8.0 Hz, 1H), 7.74 (d, J=7.6 Hz, 1H), 7.58-7.42 (m, 3H), 7.36-7.29 (m, 3H), 7.13 (d, J=7.6 Hz, 2H), 6.75 (s, 1H), 6.26 (s, 1H), 6.22 (brs, 2H), 4.54-4.52 (m, 1H), 4.25-4.13 (m, 3H), 4.02-3.45 (m, 9H), 3.26-2.35 (m, 15H), 2.07-1.75 (m, 3H), 1.63-1.51 (m, 4H), 0.93 (d, J=6.4 Hz, 6H). LC-MS: m/z 995.5 [M+H]⁺.

### Compound 170

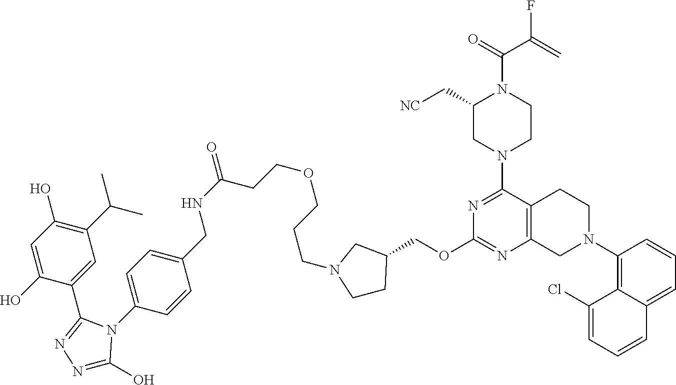

### 3-(3-((R)-3-(((7-(8-chloronaphthalen-1-yl)-4-((S)-3-(cyanomethyl)-4-(2-fluoroacryloyl)piperazin-1-yl)-5,6,7,8-tetrahydropyrido[3,4-d]pyrimidin-2-yl)oxy)methyl)pyrrolidin-1-yl)propoxy)-N-(4-(3-(2,4-dihydroxy-5-isopropylphenyl)-5-hydroxy-4H-1,2,4-triazol-4-yl)benzyl)propenamide

¹H NMR (DMSO-d₆, 400 MHz): δ 11.86 (brs, 1H), 9.59 (brs, 1H), 9.45 (brs, 1H), 8.34 (d, J=6.0 Hz, 1H), 7.92 (d, J=8.4 Hz, 1H), 7.74 (dd, J=8.0, 3.2 Hz, 1H), 7.58-7.50 (m, 2H), 7.44 (dd, J=8.0, 7.6 Hz, 1H), 7.33 (dd, J=14.8, 7.6 Hz, 1H), 7.21 (d, J=8.4 Hz, 2H), 7.09 (d, J=8.4 Hz, 2H), 6.85 (s, 1H), 6.22 (s, 1H), 5.41-5.20 (m, 2H), 4.85-4.60 (m, 1H), 4.25-3.37 (m, 15H), 3.12-2.88 (m, 6H), 2.69-2.27 (m, 9H), 2.02-1.97 (m, 1H), 1.65-1.60 (m, 2H), 1.45-1.29 (m, 3H), 0.99 (d, J=6.8 Hz, 6H). LC-MS: m/z 1042.5 [M+H]⁺.

### Compound 171

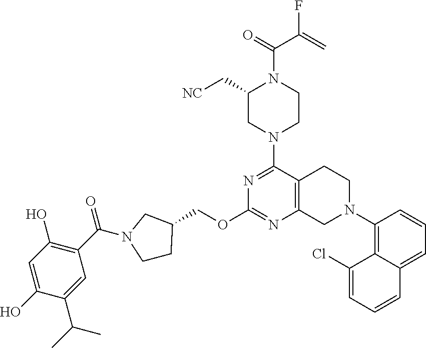 [SMILES: C=C(F)C(=O)N1CCN(c2nc(OC[C@@H]3CCN(C(=O)c4cc(C(C)C)c(O)cc4O)C3)nc3c2CCN(c2cccc4cccc(Cl)c24)C3)C[C@@H]1CC#N]

### 2-((S)-4-(7-(8-chloronaphthalen-1-yl)-2-(((R)-1-(2,4-dihydroxy-5-isopropylbenzoyl)pyrrolidin-3-yl)methoxy)-5,6,7,8-tetrahydropyrido[3,4-d]pyrimidin-4-yl)-1-(2-fluoroacryloyl)piperazin-2-yl)acetonitrile

¹H NMR (DMSO-d₆, 400 MHz): δ 10.501 (s, 1H), 9.67 (s, 1H), 7.92-7.90 (d, 1H), 7.75-7.72 (dd, 1H), 7.58-7.50 (m, 2H), 7.46-7.42 (t, 1H), 7.36-7.30 (dd, 3H), 7.04 (s, 1H), 6.31 (s, 1H), 5.41-5.40 (dd, 1H), 5.36-5.20 (d, 1H), 4.28-3.88 (m, 12H), 3.09-2.59 (m, 8H), 2.03-1.98 (m, 2H), 1.74-1.69 (m, 1H), 1.10-1.08 (d, 6H) LC-MS: m/z 768.3 [M+H]⁺.

### Compound 172

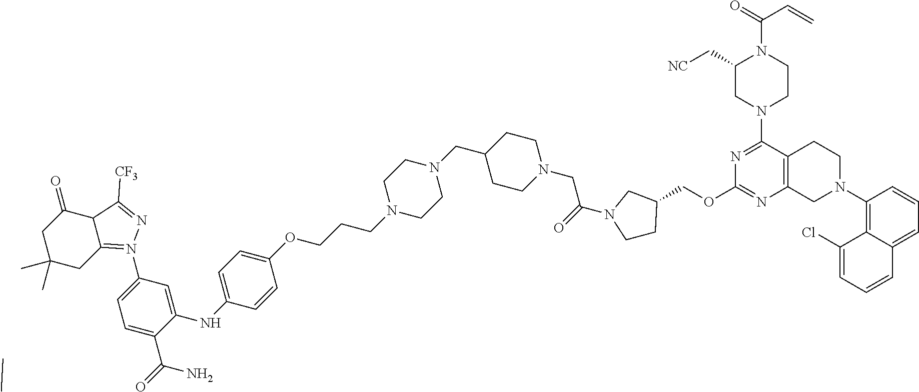

### 2-((4-(3-(4-((1-(2-((R)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)pyrrolidin-1-yl)-2-oxoethyl)piperidin-4-yl)methyl)piperidin-1-yl)propoxy)phenyl)amino)-4-(6,6-dimethyl-4-oxo-3-(trifluoromethyl)-4,5,6,7-tetrahydro-1H-indazol-1-yl)benzamide

¹H NMR (DMSO-d₆, 400 MHz): δ 10.13 (s, 1H), 7.78 (t, J=8.0 Hz, 1H), 7.68 (d, J=8.4 Hz, 1H), 7.59-7.30 (m, 9H), 7.17-6.85 (m, 5H), 6.18 (d, J=8.4 Hz, 1H), 5.75 (d, J=8.4 Hz, 1H), 4.93-4.71 (m, 1H), 4.22-3.75 (m, 8H), 2.86-2.67 (m, 8H), 2.41-2.33 (m, 10H), 2.16-1.38 (m, 17H), 1.32-0.89 (m, 17H). LC-MS: m/z 1293.6 [M+H]⁺.

### Compound 173

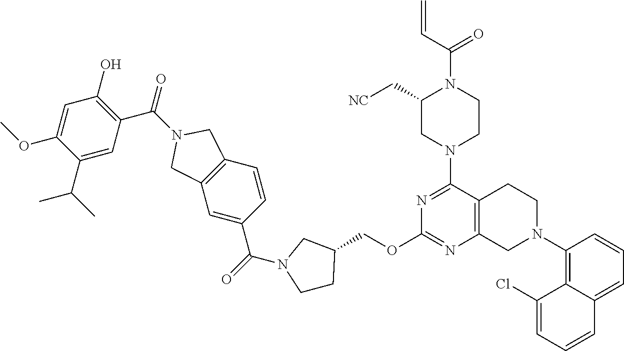 [SMILES: C=CC(=O)N1CCN(c2nc(OC[C@@H]3CCN(C(=O)c4ccc5c(c4)CN(C(=O)c4cc(C(C)C)c(OC)cc4O)C5)C3)nc3c2CCN(c2cccc4cccc(Cl)c24)C3)C[C@@H]1CC#N]

### 2-((S)-1-acryloyl-4-(7-(8-chloronaphthalen-1-yl)-2-(((R)-1-(2-(2-hydroxy-5-isopropyl-4-methoxybenzoyl)isoindoline-5-carbonyl)pyrrolidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 10.19 (brs, 1H), 7.92 (d, J=8.0 Hz, 1H), 7.74 (d, J=7.2 Hz, 1H), 7.58-7.32 (m, 7H), 6.50 (s, 1H), 6.18 (d, J=16.0 Hz, 1H), 5.77 (d, J=10.8 Hz, 1H), 4.94-4.79 (m, 1H), 4.78 (d, J=27.2 Hz, 4H), 4.39-3.89 (m, 7H), 3.78 (s, 3H), 3.71-3.50 (m, 12H), 3.16-3.01 (m, 1H), 2.67 (brs, 1H), 2.06-1.97 (m, 2H), 1.74 (brs, 1H), 1.13 (t, J=5.6 Hz, 6H). LC-MS: m/z 909.4 [M+H]⁺.

### Compound 174

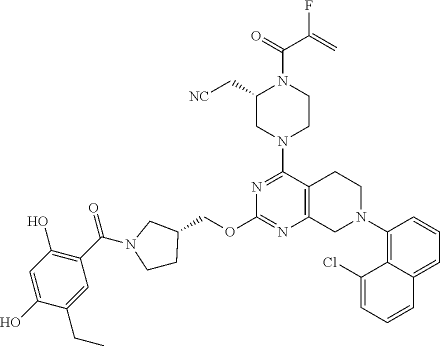 [SMILES: C=C(F)C(=O)N1CCN(c2nc(OC[C@@H]3CCN(C(=O)c4cc(CC)c(O)cc4O)C3)nc3c2CCN(c2cccc4cccc(Cl)c24)C3)C[C@@H]1CC#N]

### 2-((S)-4-(7-(8-chloronaphthalen-1-yl)-2-(((R)-1-(5-ethyl-2,4-dihydroxybenzoyl)pyrrolidin-3-yl)methoxy)-5,6,7,8-tetrahydropyrido[3,4-d]pyrimidin-4-yl)-1-(2-fluoroacryloyl)piperazin-2-yl)acetonitrile

¹H NMR (DMSO-d₆, 400 MHz): δ 7.90-7.88 (d, 1H), 7.76-7.73 (d, 2H), 7.61-7.53 (m, 2H), 7.46-7.38 (m, 1H), 7.15 (s, 1H), 6.38 (s, 1H), 5.68-5.32 (dd, 2H), 4.50-4.21 (m, 6H), 3.89-3.58 (m, 7H), 3.29-2.80 (m, 8H), 2.62-2.54 (q, 2H), 2.26-2.11 (m, 3H), 1.22-1.17 (t, 3H), 0.97-0.95 (m, 1H) LC-MS: m/z 754.3 [M+H]+

### Compound 176

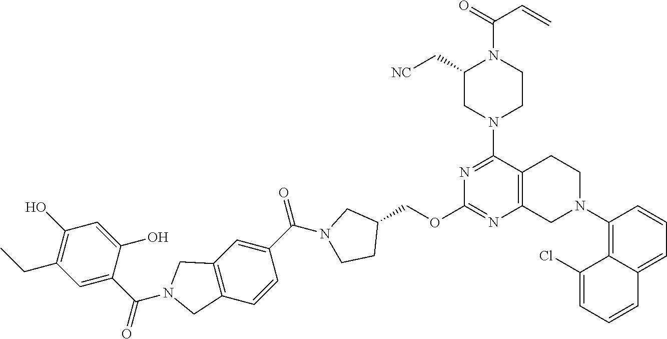 [SMILES: C=CC(=O)N1CCN(c2nc(OC[C@@H]3CCN(C(=O)c4ccc5c(c4)CN(C(=O)c4cc(CC)c(O)cc4O)C5)C3)nc3c2CCN(c2cccc4cccc(Cl)c24)C3)C[C@@H]1CC#N]

### 2-((S)-1-acryloyl-4-(7-(8-chloronaphthalen-1-yl)-2-(((R)-1-(2-(5-ethyl-2,4-dihydroxybenzoyl)isoindoline-5-carbonyl)pyrrolidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 10.13 (brs, 1H), 9.64 (brs, 1H), 7.92 (d, J=8.0 Hz, 1H), 7.74 (d, J=8.0 Hz, 1H), 7.57-7.31 (m, 7H), 7.03 (s, 1H), 6.18 (d, J=17.2 Hz, 1H), 5.77 (d, J=10.4 Hz, 1H), 4.96-4.80 (m, 1H), 4.79 (s, 4H), 4.39-3.63 (m, 13H), 3.10-3.03 (m, 3H), 2.91-2.83 (m, 1H), 2.63 (brs, 2H), 2.46-2.44 (m, 4H), 2.01-1.72 (m, 4H), 1.11-1.06 (m, 3H). LC-MS: m/z 909.4 [M+H]⁺.

### Compound 177

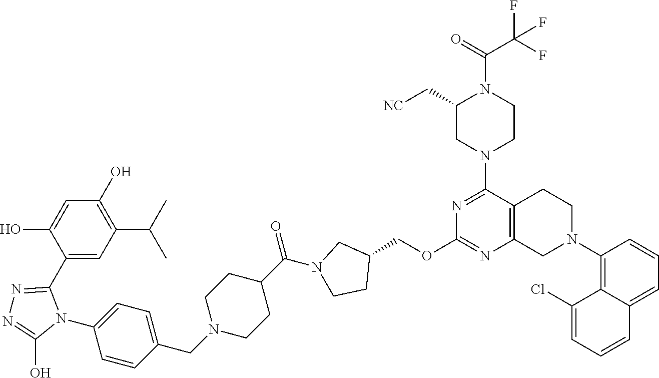 [SMILES: CC(C)c1cc(-c2nnc(O)n2-c2ccc(CN3CCC(C(=O)N4CC[C@@H](COc5nc6c(c(N7CCN(C(=O)C(F)(F)F)[C@@H](CC#N)C7)n5)CCN(c5cccc7cccc(Cl)c57)C6)C4)CC3)cc2)c(O)cc1O]

### 2-((S)-4-(7-(8-chloronaphthalen-1-yl)-2-(((R)-1-(1-(4-(3-(2,4-dihydroxy-5-isopropylphenyl)-5-hydroxy-4H-1,2,4-triazol-4-yl)benzyl)piperidine-4-carbonyl)pyrrolidin-3-yl)methoxy)-5,6,7,8-tetrahydropyrido[3,4-d]pyrimidin-4-yl)-1-(2,2,2-trifluoroacetyl)piperazin-2-yl)acetonitrile

¹H NMR (DMSO-d₆, 400 MHz): δ 11.98 (s, 1H), 9.62 (s, 1H), 9.45 (brs, 1H), 9.34 (s, 1H), 7.93 (d, J=8.0 Hz, 1H), 7.75 (d, J=8.0 Hz, 1H), 7.59-7.43 (m, 5H), 7.37-7.32 (m, 1H), 7.26 (d, J=6.0 Hz, 2H), 6.86 (s, 1H), 6.24 (s, 1H), 4.91-4.51 (m, 1H), 4.33-3.63 (m, 11H), 3.25-2.92 (m, 12H), 2.72-2.67 (m, 2H), 2.10-1.45 (m, 8H), 0.99 (d, J=6.8 Hz, 6H). LC-MS: m/z 1048.4 [M+H]⁺.

### Compound 179

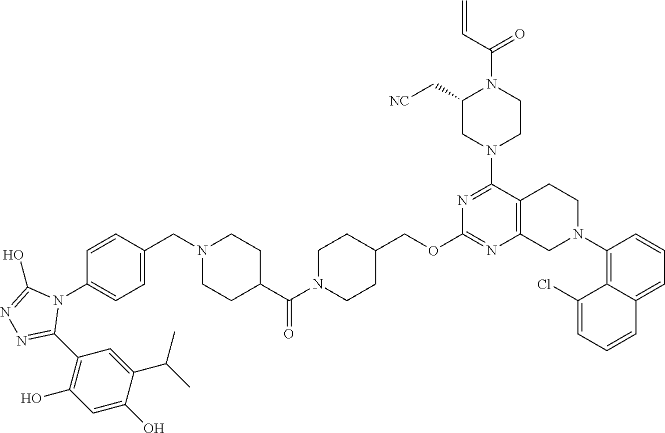 [SMILES: C=CC(=O)N1CCN(c2nc(OCC3CCN(C(=O)C4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-((1-(1-(4-(3-(2,4-dihydroxy-5-isopropylphenyl)-5-hydroxy-4H-1,2,4-triazol-4-yl)benzyl)piperidine-4-carbonyl)piperidin-4-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.84 (s, 1H), 9.61 (s, 1H), 9.43 (s, 1H), 7.92 (d, J=8.0 Hz, 1H), 7.74 (d, J=8.0 Hz, 1H), 7.52-7.45 (m, 6H), 7.12 (d, J=8.0 Hz, 2H), 6.91-6.72 (m, 2H), 6.27 (s, 1H), 6.21 (d, J=16.8 Hz, 1H), 5.77 (d, J=10.4 Hz, 1H), 4.96-4.73 (m, 1H), 4.42 (d, J=8.0 Hz, 1H), 4.32-3.81 (m, 7H), 3.51-3.42 (m, 5H), 3.08-2.77 (m, 9H), 2.01-1.32 (m, 15H), 0.92 (dd, J=6.8 Hz, 2.0 Hz, 6H). LC-MS: m/z 1020.5 [M+H]⁺.

Compound 180

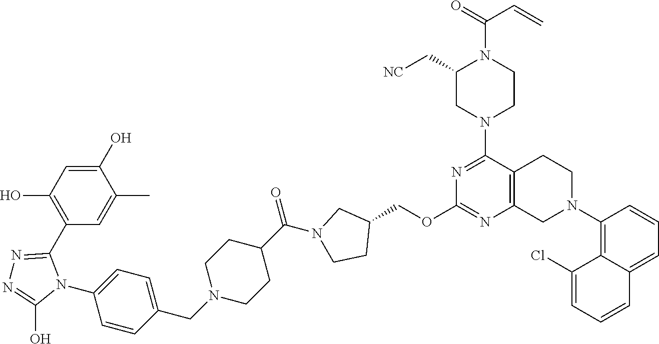 [SMILES: C=CC(=O)N1CCN(c2nc(OC[C@@H]3CCN(C(=O)C4CCN(Cc5ccc(-n6c(O)nnc6-c6cc(C)c(O)cc6O)cc5)CC4)C3)nc3c2CCN(c2cccc4cccc(Cl)c24)C3)C[C@@H]1CC#N]

### 2-((S)-1-acryloyl-4-(7-(8-chloronaphthalen-1-yl)-2-(((R)-1-(1-(4-(3-(2,4-dihydroxy-5-methylphenyl)-5-hydroxy-4H-1,2,4-triazol-4-yl)benzyl)piperidine-4-carbonyl)pyrrolidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.97 (s, 1H), 9.63 (brs, 1H), 9.52 (brs, 1H), 9.31 (brs, 1H), 7.93 (d, J=7.6 Hz, 1H), 7.75 (d, J=8.4 Hz, 1H), 7.60-7.45 (m, 5H), 7.34 (dd, J=14.0, 8.0 Hz, 1H), 7.25 (d, J=6.4 Hz, 2H), 6.95 (s, 1H), 6.85-6.82 (m, 1H), 6.22 (s, 1H), 6.19 (d, J=20.4 Hz, 1H), 5.78 (d, J=10.0 Hz, 1H), 4.95-4.75 (m, 1H), 4.41-3.95 (m, 9H), 3.80-3.70 (m, 2H), 3.36-2.84 (m, 13H), 2.71-2.57 (m, 3H), 2.10-1.64 (m, 7H), 1.97 (s, 3H). LC-MS: m/z 978.4 [M+H]⁺.

### Compound 181

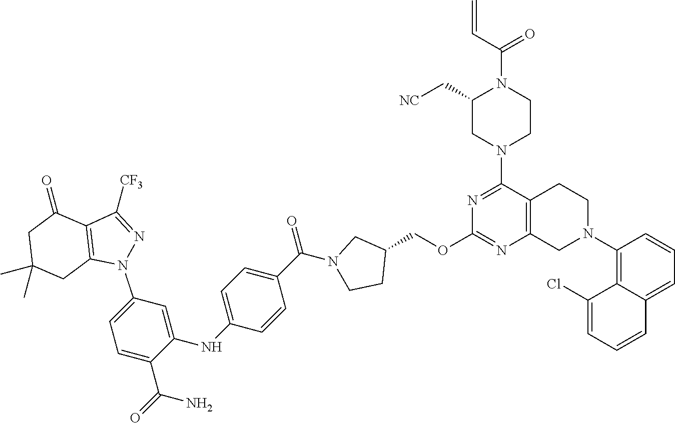 [SMILES: C=CC(=O)N1CCN(c2nc(OC[C@@H]3CCN(C(=O)c4ccc(Nc5cc(-n6nc(C(F)(F)F)c7c6CC(C)(C)CC7=O)ccc5C(N)=O)cc4)C3)nc3c2CCN(c2cccc4cccc(Cl)c24)C3)C[C@@H]1CC#N]

### 2-((4-((R)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)pyrrolidine-1-carbonyl)phenyl)amino)-4-(6,6-dimethyl-4-oxo-3-(trifluoromethyl)-4,5,6,7-tetrahydro-1H-indazol-1-yl)benzamide

¹H NMR (DMSO-d₆, 400 MHz): δ 10.33 (s, 1H), 8.26 (s, 1H), 7.94-7.90 (m, 2H), 7.75-7.73 (m, 2H), 7.57-7.42 (m, 6H), 7.34-7.27 (m, 3H), 7.14 (d, J=8.4 Hz, 1H), 6.86-6.82 (m, 1H), 6.17 (d, J=16.4 Hz, 1H), 5.76 (d, J=10.8 Hz, 2H), 4.95-4.73 (m, 1H), 4.37-3.89 (m, 6H), 3.72-3.36 (m, 6H), 3.29-2.67 (m, 10H), 2.45-2.33 (m, 3H), 2.07-2.01 (m, 1H), 1.74-1.71 (m, 1H), 1.03 (s, 6H). LC-MS: m/z 1040.4 [M+H]⁺.

### Compound 182

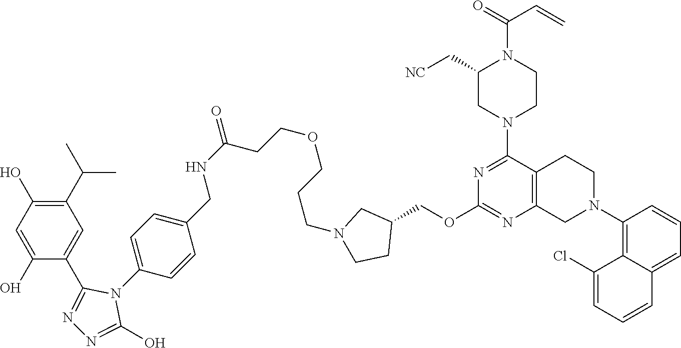 [SMILES: C=CC(=O)N1CCN(c2nc(OC[C@@H]3CCN(CCCOCCC(=O)NCc4ccc(-n5c(O)nnc5-c5cc(C(C)C)c(O)cc5O)cc4)C3)nc3c2CCN(c2cccc4cccc(Cl)c24)C3)C[C@@H]1CC#N]

### 3-(3-((R)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)pyrrolidin-1-yl)propoxy)-N-(4-(3-(2,4-dihydroxy-5-isopropylphenyl)-5-hydroxy-4H-1,2,4-triazol-4-yl)benzyl)propanamide

¹H NMR (DMSO-d₆, 400 MHz): δ 7.85 (d, J=8.4 Hz, 1H), 7.70 (d, J=7.6 Hz, 1H), 7.55-7.49 (m, 2H), 7.41-7.37 (m, 3H), 7.32 (dd, J=10.8, 8.0 Hz, 1H), 7.25 (d, J=8.4 Hz, 2H), 6.87-6.73 (m, 2H), 6.32 (d, J=16.8 Hz, 1H), 6.27 (s, 1H), 5.86 (d, J=10.8 Hz, 1H), 5.11-5.06 (m, 1H), 4.41-4.06 (m, 7H), 3.74-3.58 (m, 7H), 3.21-2.64 (m, 15H), 2.52 (t, J=6.0 Hz, 2H), 2.20-2.18 (m, 1H), 2.06-1.83 (m, 4H), 0.93 (d, J=6.8 Hz, 6H). LC-MS: m/z 1024.5 [M+H]⁺.

### Compound 183

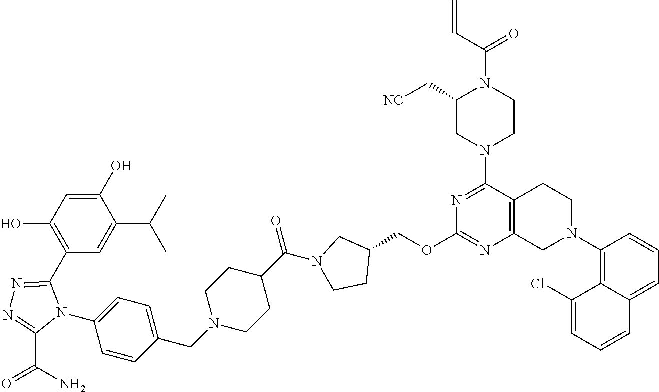 [SMILES: C=CC(=O)N1CCN(c2nc(OC[C@@H]3CCN(C(=O)C4CCN(Cc5ccc(-n6c(C(N)=O)nnc6-c6cc(C(C)C)c(O)cc6O)cc5)CC4)C3)nc3c2CCN(c2cccc4cccc(Cl)c24)C3)C[C@@H]1CC#N]

### 4-(4-((4-((R)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)pyrrolidine-1-carbonyl)piperidin-1-yl)methyl)phenyl)-5-(2,4-dihydroxy-5-isopropylphenyl)-4H-1,2,4-triazole-3-carboxamide

¹H NMR (DMSO-d₆, 400 MHz): δ 10.63 (s, 1H), 9.76 (s, 1H), 8.25 (s, 2H), 7.92-7.90 (dd, 1H), 7.72 (s, 2H), 7.58-7.52 (m, 2H), 7.46-7.29 (m, 6H), 6.82 (s, 1H), 6.56 (s, 1H), 6.35 (s, 1H), 5.78-5.76 (dd, 1H), 4.94-4.77 (d, 1H), 4.63-3.21 (m, 10H), 3.20-2.60 (m, 10H) 2.57 (s, 2H), 2.38-1.60 (m, 10H), 0.79 (s, 6H) LC-MS: m/z 1033.5[M+H]⁺.

### Compound 184

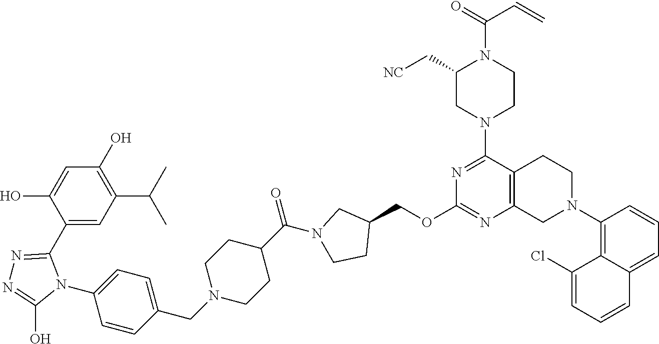 [SMILES: C=CC(=O)N1CCN(c2nc(OC[C@H]3CCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N]

### 2-((S)-1-acryloyl-4-(7-(8-chloronaphthalen-1-yl)-2-(((S)-1-(1-(4-(3-(2,4-dihydroxy-5-isopropylphenyl)-5-hydroxy-4H-1,2,4-triazol-4-yl)benzyl)piperidine-4-carbonyl)pyrrolidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.92 (s, 1H), 9.59 (s, 1H), 9.41 (s, 1H), 7.92-7.90 (dd, 1H), 7.73 (s, 1H), 7.58-7.52 (dd, 2H), 7.46-7.44 (t, 1H), 7.29-7.27 (m, 4H), 7.13-7.11 (d, 2H), 6.89-6.83 (m, 1H), 6.74 (s, 1H), 6.26 (s, 1H), 6.21-6.17 (d, 1H), 5.79-5.77 (d, 1H) 4.98-4.76 (m, 1H), 4.17-3.41 (m, 10H), 3.25-2.67 (m, 11H) 2.54 (s, 2H), 2.32-1.52 (m, 11H), 0.92-0.91 (d, 6H) LC-MS: m/z 1006.4[M+H]⁺.

### Compound 185

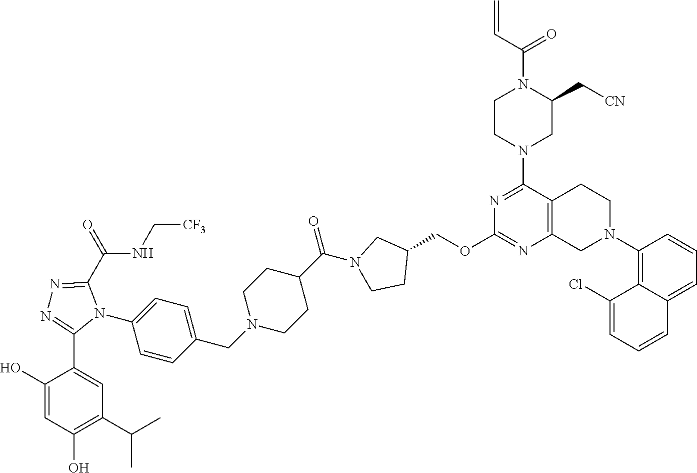 [SMILES: C=CC(=O)N1CCN(c2nc(OC[C@@H]3CCN(C(=O)C4CCN(Cc5ccc(-n6c(C(=O)NCC(F)(F)F)nnc6-c6cc(C(C)C)c(O)cc6O)cc5)CC4)C3)nc3c2CCN(c2cccc4cccc(Cl)c24)C3)C[C@@H]1CC#N]

### 4-(4-((4-((R)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)pyrrolidine-1-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide

¹H NMR (DMSO-d₆, 400 MHz): δ 9.75 (s, 1H), 9.59 (t, J=6.4 Hz, 1H), 7.92 (d, J=8.4 Hz, 1H), 7.74 (d, J=8.0 Hz, 1H), 7.59-7.30 (m, 8H), 6.81 (s, 1H), 6.61 (s, 1H), 6.35 (s, 1H), 6.18 (d, J=16.8 Hz, 1H), 5.77 (d, J=10.8 Hz, 1H), 4.98-4.64 (m, 1H), 4.22-3.34 (m, 14H), 3.24-2.67 (m, 12H), 2.08-1.60 (m, 9H), 1.23 (s, 2H), 0.82-0.80 (m, 6H). LC-MS: m/z 1115.4 [M+H]⁺.

### Compound 186

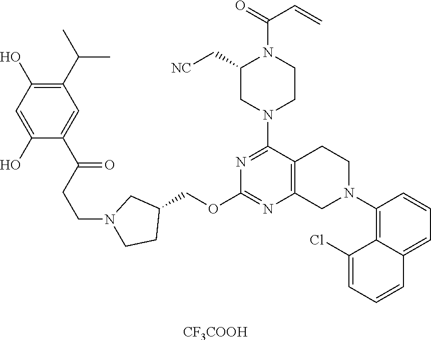 [SMILES: C=CC(=O)N1CCN(c2nc(OC[C@@H]3CCN(CCC(=O)c4cc(C(C)C)c(O)cc4O)C3)nc3c2CCN(c2cccc4cccc(Cl)c24)C3)C[C@@H]1CC#N ; "CF3COOH"]

### 2-((S)-1-acryloyl-4-(7-(8-chloronaphthalen-1-yl)-2-(((R)-1-(3-(2,4-dihydroxy-5-isopropylphenyl)-3-oxopropyl)pyrrolidin-3-yl)methoxy)-5,6,7,8-tetrahydropyrido[3,4-d]pyrimidin-4-yl)piperazin-2-yl)acetonitrile, trifluoroacetic acid

¹H NMR (DMSO-d₆, 400 MHz): δ 11.84 (s, 1H), 10.69 (s, 1H), 9.50 (s, 1H), 7.93 (d, J=8.4 Hz, 1H), 7.75 (dd, J=8.0, 3.6 Hz, 1H), 7.59-7.51 (m, 3H), 7.45 (dd, J=8.0, 7.2 Hz, 1H), 7.34 (dd, J=16.0, 7.6 Hz, 1H), 6.85-6.83 (m, 1H), 6.35 (s, 1H), 6.19 (d, J=16.4 Hz, 1H), 5.78 (d, J=11.6 Hz, 1H), 4.97-4.74 (m, 1H), 4.42-3.70 (m, 17H), 3.42-2.67 (m, 8H), 2.40-1.71 (m, 3H), 1.17 (d, J=6.8 Hz, 6H). LC-MS: m/z 778.5 [M-CF₃COOH+H]⁺.

### Compound 187

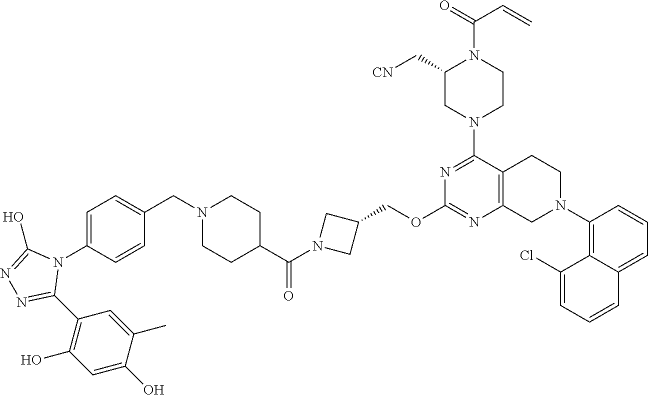

### (S)-2-(1-acryloyl-4-(7-(8-chloronaphthalen-1-yl)-2-((1-(1-(4-(3-(2,4-dihydroxy-5-methylphenyl)-5-hydroxy-4H-1,2,4-triazol-4-yl)benzyl)piperidine-4-carbonyl)azetidin-3-yl)methoxy)-5,6,7,8-tetrahydropyrido[3,4-d]pyrimidin-4-yl)piperazin-2-yl)acetonitrile, trifluoroacetic acid

¹H NMR (400 MHz, MeOD) δ 7.83 (d, J=8.1 Hz, 1H), 7.70 (d, J=8.3 Hz, 1H), 7.51 (d, J=8.0 Hz, 4H), 7.38 (t, J=8.3 Hz, 4H), 6.90 (s, 1H), 6.88-6.74 (m, 1H), 6.30 (d, J=16.5 Hz, 1H), 6.17 (s, 1H), 5.85 (s, 1H), 5.34 (t, J=4.6 Hz, 1H), 4.58 (m, 1H), 4.41 (m, 1H), 4.35 (m, 1H), 4.30 (m, 3H), 4.20 (m, 1H), 4.16 (m, 1H), 4.12 (m, 1H), 4.08-4.02 (m, 1H), 3.90 (m, 1H), 3.81 (m, 1H), 3.75 (m, 1H), 3.66-3.59 (m, 2H), 3.54 (m, 1H), 3.50-3.46 (m, 2H), 3.03 (m, 2H), 2.92 (m, 1H), 2.80 (m, 1H), 2.61 (m, 1H), 2.35 (m, 1H), 2.19 (m, 1H), 2.02 (m, 3H), 1.99 (s, 3H), 1.85 (m, 1H), 1.60 (m, 3H). LC-MS: m/z 964.3 [M+H]⁺.

### Compound 188

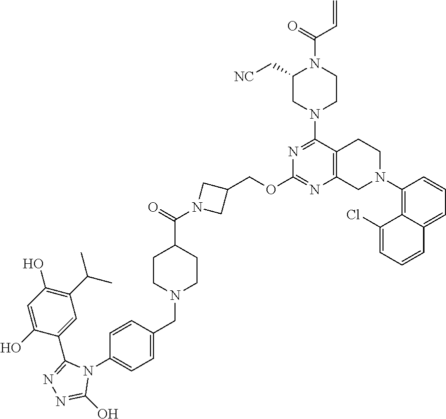 [SMILES: C=CC(=O)N1CCN(c2nc(OCC3CN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N]

### (S)-2-(1-acryloyl-4-(7-(8-chloronaphthalen-1-yl)-2-((1-(1-(4-(3-(2,4-dihydroxy-5-isopropylphenyl)-5-hydroxy-4H-1,2,4-triazol-4-yl)benzyl)piperidine-4-carbonyl)azetidin-3-yl)methoxy)-5,6,7,8-tetrahydropyrido[3,4-d]pyrimidin-4-yl)piperazin-2-yl)acetonitrile, trifluoroacetic acid

¹H NMR (400 MHz, MeOD) δ 7.84 (d, J=8.3 Hz, 1H), 7.72 (d, J=8.0 Hz, 1H), 7.58-7.48 (m, 4H), 7.42-7.32 (m, 4H), 6.88-6.72 (m, 2H), 6.30 (m, 1H), 6.21 (s, 1H), 5.85 (d, J=10.7 Hz, 1H), 4.65 (d, J=5.7 Hz, 2H), 4.52-4.45 (m, 1H), 4.44-4.30 (m, 5H), 4.20-4.09 (m, 3H), 3.91-3.85 (m, 1H), 3.82-3.75 (m, 1H), 3.66-3.57 (m, 2H), 3.55-3.46 (m, 3H), 3.26-3.15 (m, 3H), 3.14-2.99 (m, 5H), 2.96-2.90 (m, 1H), 2.84-2.77 (m, 1H), 2.66-2.54 (m, 1H), 2.05-1.79 (m, 5H), 1.00 (d, J=6.9 Hz, 6H). LC-MS: m/z 992.4 [M+H]⁺.

### Compound 189

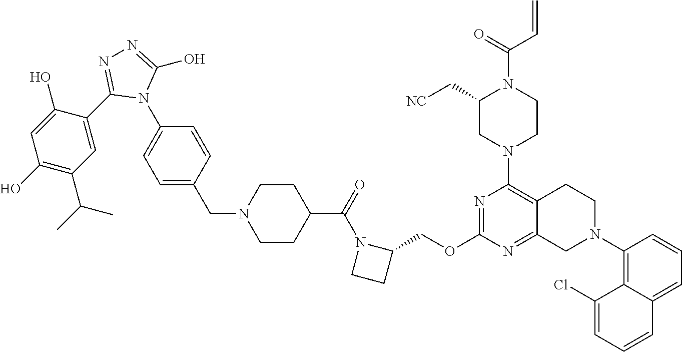 [SMILES: C=CC(=O)N1CCN(c2nc(OC[C@@H]3CCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N]

### 2-((S)-1-acryloyl-4-(7-(8-chloronaphthalen-1-yl)-2-(((S)-1-(1-(4-(3-(2,4-dihydroxy-5-isopropylphenyl)-5-hydroxy-4H-1,2,4-triazol-4-yl)benzyl)piperidine-4-carbonyl)azetidin-2-yl)methoxy)-5,6,7,8-tetrahydropyrido[3,4-d]pyrimidin-4-yl)piperazin-2-yl)acetonitrile, trifluoroacetic acid

¹H NMR (400 MHz, MeOD) δ 7.90-7.60 (m, 2H), 7.58-7.45 (m, 3H), 7.40-7.20 (m, 4H), 6.96-6.54 (m, 3H), 6.37-6.19 (m, 2H), 5.83 (d, J=12.4 Hz, 1H), 5.15-4.97 (m, 1H), 4.72-4.50 (m, 3H), 4.42-4.04 (m, 7H), 3.99-3.94 (m, 1H), 3.80-3.45 (m, 5H), 3.22-2.90 (m, 8H), 2.82-2.25 (m, 4H), 2.10-1.75 (m, 5H), 1.00 (d, J=6.5 Hz, 3H), 0.94-0.77 (m, 3H). LC-MS: m/z 992.7 [M+H]⁺.

### Compound 190

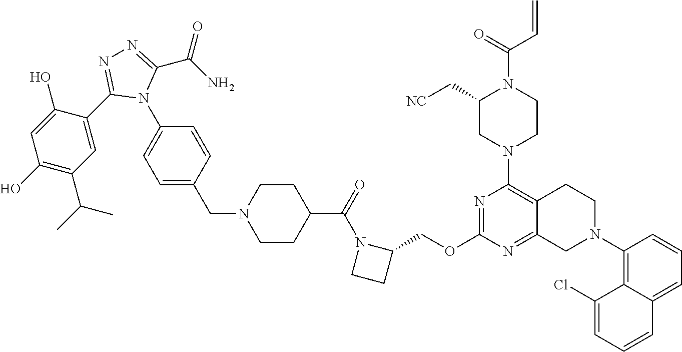 [SMILES: C=CC(=O)N1CCN(c2nc(OC[C@@H]3CCN3C(=O)C3CCN(Cc4ccc(-n5c(C(N)=O)nnc5-c5cc(C(C)C)c(O)cc5O)cc4)CC3)nc3c2CCN(c2cccc4cccc(Cl)c24)C3)C[C@@H]1CC#N]

### 4-(4-((4-((S)-2-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)azetidine-1-carbonyl)piperidin-1-yl)methyl)phenyl)-5-(2,4-dihydroxy-5-isopropylphenyl)-4H-1,2,4-triazole-3-carboxamide

¹H NMR (400 MHz, MeOD) δ 7.89-7.67 (m, 1.5H), 7.61-7.46 (m, 4H), 7.41-7.35 (m, 2H), 7.28-7.14 (m, 1.5H), 6.93-6.67 (m, 2H), 6.65-6.54 (m, 0.5H), 6.49-6.24 (m, 2.5H), 5.84 (d, J=11.6 Hz, 1H), 4.67-4.60 (m, 2H), 4.40-4.31 (m, 3H), 4.25-4.12 (m, 3H), 4.00-3.96 (m, 1H), 3.81-3.74 (m, 1H), 3.63-3.53 (m, 3H), 3.11-2.99 (m, 4H), 2.96-2.86 (m, 2H), 2.81-2.52 (m, 4H), 2.23-2.14 (m, 2H), 2.05-1.99 (m, 4H), 1.63-1.56 (m, 2H), 0.90 (m, 6H), 0.85-0.73 (m, 3H). LC-MS: m/z 1019.8 [M+H]⁺.

### Compound 191

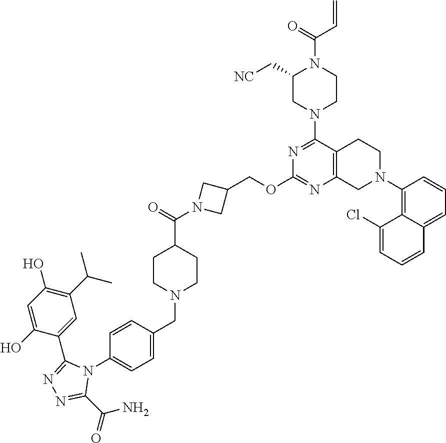

### (S)-4-(4-((4-(3-(((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)azetidine-1-carbonyl)piperidin-1-yl)methyl)phenyl)-5-(2,4-dihydroxy-5-isopropylphenyl)-4H-1,2,4-triazole-3-carboxamide

¹H NMR (400 MHz, MeOD) δ 7.84 (d, J=8.8 Hz, 1H), 7.71 (d, J=8.5 Hz, 1H), 7.61-7.43 (m, 6H), 7.41-7.32 (m, 2H), 6.88-6.69 (m, 2H), 6.33-6.22 (m, 2H), 5.84 (d, J=10.8 Hz, 1H), 4.74-4.52 (m, 3H), 4.46-4.23 (m, 6H), 4.21-4.06 (m, 3H), 3.91-3.85 (m, 1H), 3.82-3.65 (m, 2H), 3.63-3.39 (m, 5H), 3.25-2.88 (m, 8H), 2.83-2.59 (m, 2H), 2.04-1.81 (m, 4H), 0.93 (d, J=6.9 Hz, 6H). LC-MS: m/z 1019.3 [M+H]⁺.

### Compound 192

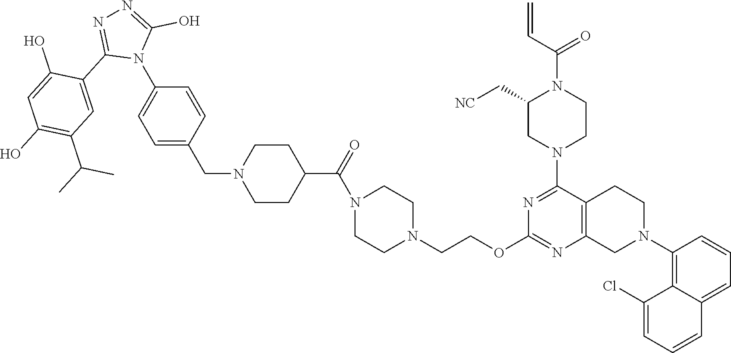 [SMILES: C=CC(=O)N1CCN(c2nc(OCCN3CCN(C(=O)C4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(1-(4-(3-(2,4-dihydroxy-5-isopropylphenyl)-5-hydroxy-4H-1,2,4-triazol-4-yl)benzyl)piperidine-4-carbonyl)piperazin-1-yl)ethoxy)-5,6,7,8-tetrahydropyrido[3,4-d]pyrimidin-4-yl)piperazin-2-yl)acetonitrile, trifluoroacetic acid

¹H NMR (400 MHz, MeOD) δ 7.84 (dd, J=8.3, 1.0 Hz, 1H), 7.70 (d, J=8.0 Hz, 1H), 7.58-7.47 (m, 4H), 7.41-7.29 (m, 4H), 6.90-6.73 (m, 2H), 6.29 (d, J=17.1 Hz, 1H), 6.22 (s, 1H), 5.84 (d, J=10.4 Hz, 1H), 4.80-4.69 (m, 3H), 4.40-4.05 (m, 6H), 3.96-3.69 (m, 5H), 3.66-3.36 (m, 11H), 3.24-3.15 (m, 2H), 3.14-2.86 (m, 7H), 2.80-2.67 (m, 1H), 2.17-1.79 (m, 4H), 1.01 (d, J=6.9 Hz, 6H). LC-MS: m/z 1035.4 [M+H]⁺.

### Compound 193

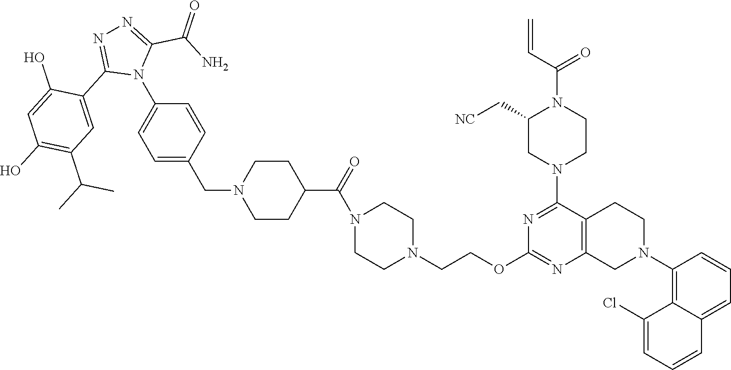 [SMILES: C=CC(=O)N1CCN(c2nc(OCCN3CCN(C(=O)C4CCN(Cc5ccc(-n6c(C(N)=O)nnc6-c6cc(C(C)C)c(O)cc6O)cc5)CC4)CC3)nc3c2CCN(c2cccc4cccc(Cl)c24)C3)C[C@@H]1CC#N]

### (S)-4-(4-((4-(4-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperazine-1-carbonyl)piperidin-1-yl)methyl)phenyl)-5-(2,4-dihydroxy-5-isopropylphenyl)-4H-1,2,4-triazole-3-carboxamide

¹H NMR (400 MHz, MeOD) δ 7.84 (d, J=7.4 Hz, 1H), 7.72 (d, J=8.1 Hz, 1H), 7.62 (d, J=8.3 Hz, 2H), 7.57-7.47 (m, 4H), 7.41-7.32 (m, 2H), 6.85-6.70 (m, 2H), 6.36-6.23 (m, 2H), 5.85 (d, J=10.3 Hz, 1H), 4.45-4.26 (m, 5H), 4.06-3.74 (m, 6H), 3.71-3.54 (m, 6H), 3.52-3.39 (m, 6H), 3.26-3.19 (m, 2H), 3.15-2.92 (m, 7H), 2.87-2.77 (m, 2H), 2.07-1.88 (m, 5H), 0.93 (d, J=6.9 Hz, 6H). LC-MS: m/z 1062.4 [M+H]⁺.

### Compound 194

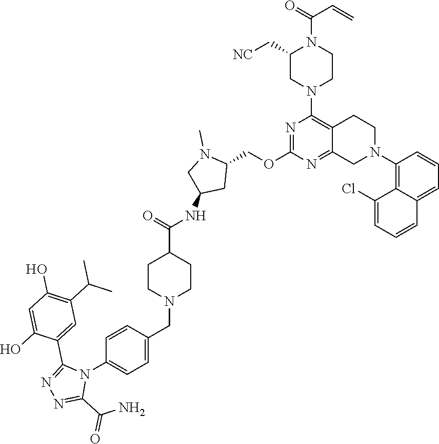 [SMILES: C=CC(=O)N1CCN(c2nc(OC[C@@H]3C[C@@H](NC(=O)C4CCN(Cc5ccc(-n6c(C(N)=O)nnc6-c6cc(C(C)C)c(O)cc6O)cc5)CC4)CN3C)nc3c2CCN(c2cccc4cccc(Cl)c24)C3)C[C@@H]1CC#N]

### N-((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-1-(4-(3-carbamoyl-5-(2,4-dihydroxy-5-isopropylphenyl)-4H-1,2,4-triazol-4-yl)benzyl)piperidine-4-carboxamide

¹H NMR (400 MHz, MeOD) δ 7.84 (d, J=7.8 Hz, 1H), 7.71 (d, J=8.2 Hz, 1H), 7.61 (d, J=8.4 Hz, 2H), 7.57-7.47 (m, 4H), 7.41-7.32 (m, 2H), 6.97-6.70 (m, 2H), 6.38-6.22 (m, 2H), 5.84 (d, J=10.8 Hz, 1H), 4.69-4.62 (m, 2H), 4.53-4.11 (m, 9H), 4.05-3.97 (m, 1H), 3.84-3.71 (m, 2H), 3.61-3.51 (m, 3H), 3.26-3.18 (m, 3H), 3.15-2.90 (m, 9H), 2.81-2.70 (m, 1H), 2.63-2.51 (m, 1H), 2.46-2.33 (m, 2H), 2.21-2.04 (m, 3H), 1.98-1.90 (m, 2H), 0.97-0.88 (m, 6H). LC-MS: m/z 1062.4 [M+H]⁺.

### Compound 195

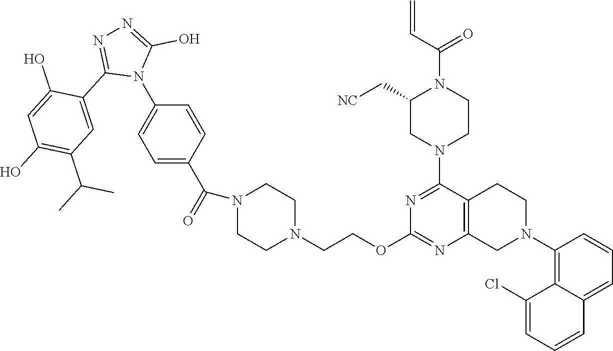 [SMILES: C=CC(=O)N1CCN(c2nc(OCCN3CCN(C(=O)c4ccc(-n5c(O)nnc5-c5cc(C(C)C)c(O)cc5O)cc4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(4-(3-(2,4-dihydroxy-5-isopropylphenyl)-5-hydroxy-4H-1,2,4-triazol-4-yl)benzoyl)piperazin-1-yl)ethoxy)-5,6,7,8-tetrahydropyrido[3,4-d]pyrimidin-4-yl)piperazin-2-yl)acetonitrile, trifluoroacetic acid

¹H NMR (400 MHz, MeOD) δ 7.84 (d, J=8.2 Hz, 1H), 7.72 (d, J=8.1 Hz, 1H), 7.59-7.47 (m, 4H), 7.42-7.29 (m, 4H), 6.92-6.72 (m, 2H), 6.30 (d, J=17.0 Hz, 1H), 6.21 (s, 1H), 5.84 (d, J=10.1 Hz, 1H), 4.78-4.65 (m, 1H), 4.54-4.29 (m, 3H), 4.11 (m, 1H), 4.03-3.65 (m, 8H), 3.65-3.40 (m, 7H), 3.28-2.88 (m, 5H), 2.84-2.68 (m, 1H), 2.22-1.97 (m, 1H), 1.82-1.42 (m, 1H), 1.02 (d, J=6.9 Hz, 6H). LC-MS: m/z 938.8 [M+H]⁺.

### Compound 196

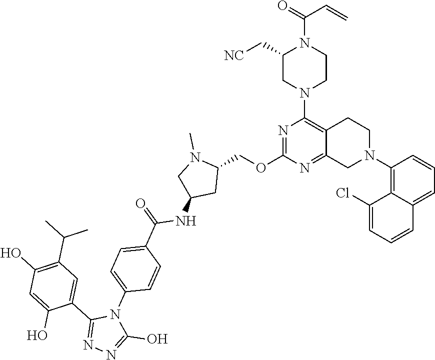 [SMILES: C=CC(=O)N1CCN(c2nc(OC[C@@H]3C[C@@H](NC(=O)c4ccc(-n5c(O)nnc5-c5cc(C(C)C)c(O)cc5O)cc4)CN3C)nc3c2CCN(c2cccc4cccc(Cl)c24)C3)C[C@@H]1CC#N]

### N-((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-4-(3-(2,4-dihydroxy-5-isopropylphenyl)-5-hydroxy-4H-1,2,4-triazol-4-yl)benzamide, trifluoroacetic acid

¹H NMR (400 MHz, MeOD) δ 7.86 (dd, J=20.5, 8.1 Hz, 3H), 7.69 (d, J=9.0 Hz, 1H), 7.55-7.48 (m, 2H), 7.41-7.30 (m, 4H), 6.93-6.74 (m, 2H), 6.29 (d, J=16.9 Hz, 1H), 6.21 (s, 1H), 5.83 (d, J=9.4 Hz, 1H), 4.66-4.60 (m, 2H), 4.36-4.04 (m, 7H), 3.77-3.48 (m, 5H), 3.26-3.03 (m, 9H), 2.95-2.89 (m, 1H), 2.78-2.69 (m, 1H), 2.53-2.45 (m, 2H), 1.02 (d, 6H). LC-MS: m/z 938.3 [M+H]⁺.

### Compound 197

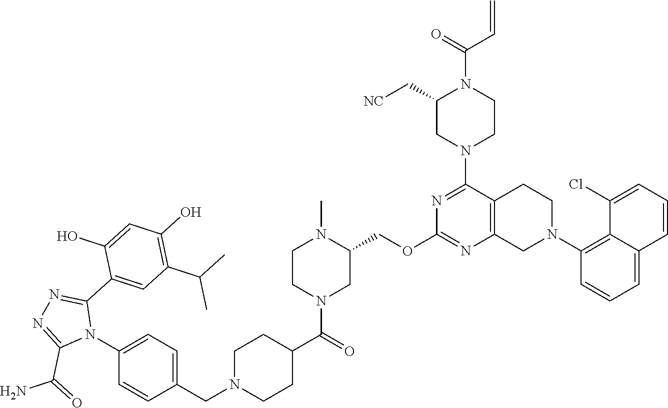 [SMILES: C=CC(=O)N1CCN(c2nc(OC[C@@H]3CN(C(=O)C4CCN(Cc5ccc(-n6c(C(N)=O)nnc6-c6cc(C(C)C)c(O)cc6O)cc5)CC4)CCN3C)nc3c2CCN(c2cccc4cccc(Cl)c24)C3)C[C@@H]1CC#N]

### 4-(4-((4-((S)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-4-methylpiperazine-1-carbonyl)piperidin-1-yl)methyl)phenyl)-5-(2,4-dihydroxy-5-isopropylphenyl)-4H-1,2,4-triazole-3-carboxamide, trifluoroacetic acid

¹H NMR (400 MHz, MeOD) δ 7.83 (d, J=8.4 Hz, 1H), 7.69 (dd, J=7.6, 3.5 Hz, 1H), 7.63-7.41 (m, 6H), 7.40-7.30 (m, 2H), 6.87-6.66 (m, 2H), 6.30 (d, J=12.4 Hz, 2H), 5.84 (d, J=10.6 Hz, 1H), 4.76-4.51 (m, 3H), 4.43-4.02 (m, 7H), 3.82-3.45 (m, 9H), 3.27-3.17 (m, 4H), 3.15-2.82 (m, 10H), 2.79-2.68 (m, 1H), 2.26-1.75 (m, 5H), 0.93 (d, J=6.2 Hz, 6H). LC-MS: m/z 1060.5 [M+H]⁺.

### Compound 198

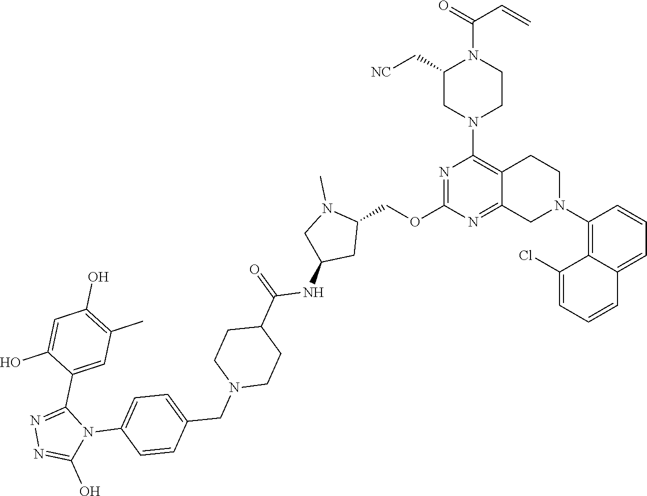 [SMILES: C=CC(=O)N1CCN(c2nc(OC[C@@H]3C[C@@H](NC(=O)C4CCN(Cc5ccc(-n6c(O)nnc6-c6cc(C)c(O)cc6O)cc5)CC4)CN3C)nc3c2CCN(c2cccc4cccc(Cl)c24)C3)C[C@@H]1CC#N]

### N-((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-1-(4-(3-(2,4-dihydroxy-5-methylphenyl)-5-hydroxy-4H-1,2,4-triazol-4-yl)benzyl)piperidine-4-carboxamide, trifluoroacetic acid

¹H NMR (400 MHz, MeOD) δ 7.84 (d, J=9.1 Hz, 1H), 7.70 (d, J=8.2 Hz, 1H), 7.56-7.47 (m, 4H), 7.44-7.28 (m, 4H), 6.97-6.69 (m, 2H), 6.29 (d, J=16.4 Hz, 1H), 6.18 (s, 1H), 5.83 (d, J=11.4 Hz, 1H), 5.11-4.97 (m, 1H), 4.66-4.43 (m, 4H), 4.39-4.26 (m, 4H), 4.16-3.99 (m, 3H), 3.80-3.70 (m, 3H), 3.66-3.58 (m, 2H), 3.55-3.48 (m, 2H), 3.24-3.19 (m, 2H), 3.13-3.06 (m, 5H), 3.04-3.00 (m, 1H), 2.97-2.86 (m, 2H), 2.81-2.67 (m, 2H), 2.46-2.33 (m, 2H), 2.06-1.98 (m, 8H), 1.47-1.40 (m, 4H). LC-MS: m/z 1007.3 [M+H]⁺.

### Compound 199

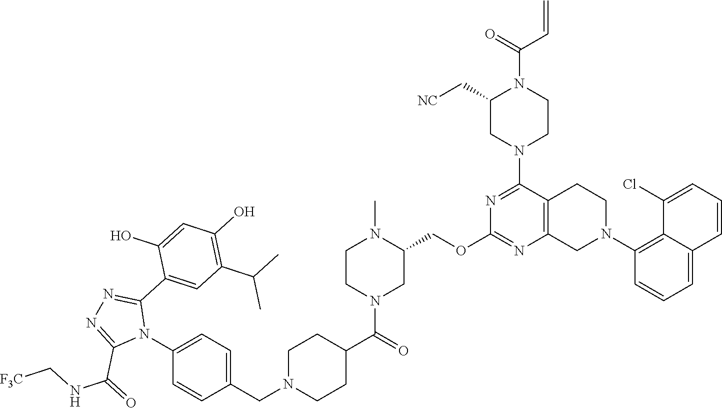 [SMILES: C=CC(=O)N1CCN(c2nc(OC[C@@H]3CN(C(=O)C4CCN(Cc5ccc(-n6c(C(=O)NCC(F)(F)F)nnc6-c6cc(C(C)C)c(O)cc6O)cc5)CC4)CCN3C)nc3c2CCN(c2cccc4cccc(Cl)c24)C3)C[C@@H]1CC#N]

### 4-(4-((4-((S)-3-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-4-methylpiperazine-1-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide

¹H NMR (400 MHz, MeOD) δ 7.84 (dd, J=10.1, 5.7 Hz, 1H), 7.69 (dd, J=9.0, 2.6 Hz, 1H), 7.64-7.44 (m, 6H), 7.42-7.30 (m, 2H), 6.88-6.70 (m, 2H), 6.30 (d, J=14.2 Hz, 2H), 5.84 (d, J=10.4 Hz, 1H), 4.77-4.52 (m, 3H), 4.45-4.06 (m, 7H), 3.98 (dd, J=18.6, 9.3 Hz, 2H), 3.79-3.46 (m, 9H), 3.27-3.16 (m, 4H), 3.15-2.99 (m, 8H), 2.95-2.65 (m, 3H), 2.13-1.83 (m, 5H), 0.94 (d, J=6.7 Hz, 6H). LC-MS: m/z 1044.5 [M+H]⁺.

### Compound 200

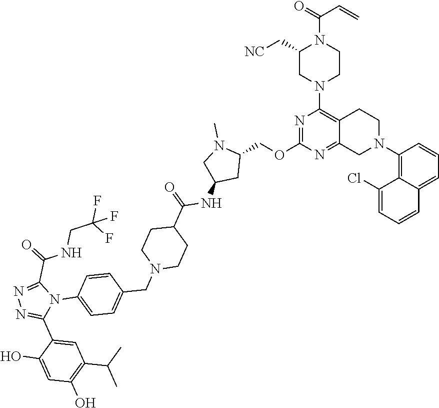 [SMILES: C=CC(=O)N1CCN(c2nc(OC[C@@H]3C[C@@H](NC(=O)C4CCN(Cc5ccc(-n6c(C(=O)NCC(F)(F)F)nnc6-c6cc(C(C)C)c(O)cc6O)cc5)CC4)CN3C)nc3c2CCN(c2cccc4cccc(Cl)c24)C3)C[C@@H]1CC#N]

### N-((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-1-(4-(3-(2,4-dihydroxy-5-isopropylphenyl)-5-((2,2,2-trifluoroethyl)carbamoyl)-4H-1,2,4-triazol-4-yl)benzyl)piperidine-4-carboxamide

¹H NMR (400 MHz, MeOD) δ 7.81 (d, J=9.0 Hz, 1H), 7.67 (d, J=10.3 Hz, 1H), 7.53-7.46 (m, 4H), 7.38-7.29 (m, 4H), 6.93-6.75 (m, 1H), 6.67 (s, 1H), 6.33-6.24 (m, 2H), 5.82 (d, J=11.7 Hz, 1H), 4.39-4.26 (m, 5H), 4.21-4.08 (m, 2H), 4.01-3.94 (m, 2H), 3.77-3.62 (m, 2H), 3.57-3.55 (m, 2H), 3.38-3.34 (m, 2H), 3.22-3.07 (m, 4H), 3.03-2.86 (m, 6H), 2.73-2.64 (m, 1H), 2.47 (s, 3H), 2.24-2.17 (m, 2H), 2.13-2.04 (m, 3H), 2.03-2.03 (m, 3H), 1.96-1.78 (m, 3H), 0.86 (d, J=6.9 Hz, 6H). LC-MS: m/z 1144.4 [M+H]⁺.

### Compound 201

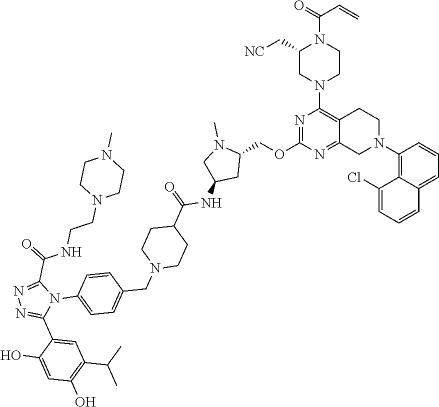 [SMILES: C=CC(=O)N1CCN(c2nc(OC[C@@H]3C[C@@H](NC(=O)C4CCN(Cc5ccc(-n6c(C(=O)NCCN7CCN(C)CC7)nnc6-c6cc(C(C)C)c(O)cc6O)cc5)CC4)CN3C)nc3c2CCN(c2cccc4cccc(Cl)c24)C3)C[C@@H]1CC#N]

### N-((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-1-(4-(3-(2,4-dihydroxy-5-isopropylphenyl)-5-((2-(4-methylpiperazin-1-yl)ethyl)carbamoyl)-4H-1,2,4-triazol-4-yl)benzyl)piperidine-4-carboxamide

¹H NMR (400 MHz, MeOD) δ 7.81 (d, J=8.1 Hz, 1H), 7.67 (d, J=10.3 Hz, 1H), 7.53-7.44 (m, 4H), 7.39-7.28 (m, 4H), 6.92-6.74 (m, 1H), 6.66 (s, 1H), 6.36-6.23 (m, 2H), 5.82 (d, J=10.8 Hz, 1H), 4.45-4.24 (m, 5H), 4.20-4.03 (m, 2H), 3.77-3.53 (m, 5H), 3.46-3.35 (m, 4H), 3.23-3.07 (m, 4H), 3.01-2.89 (m, 5H), 2.74-2.44 (m, 13H), 2.28 (s, 3H), 2.24-2.02 (m, 6H), 1.95-1.69 (m, 6H), 0.86 (d, J=6.9 Hz, 6H). LC-MS: m/z 1188.6 [M+H]⁺.

### Compound 202

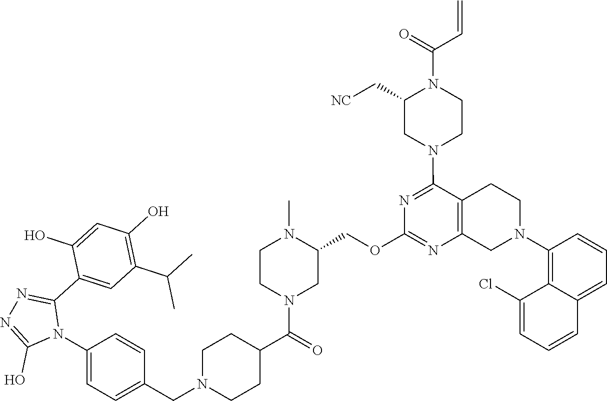 [SMILES: C=CC(=O)N1CCN(c2nc(OC[C@@H]3CN(C(=O)C4CCN(Cc5ccc(-n6c(O)nnc6-c6cc(C(C)C)c(O)cc6O)cc5)CC4)CCN3C)nc3c2CCN(c2cccc4cccc(Cl)c24)C3)C[C@@H]1CC#N]

### 2-((S)-1-acryloyl-4-(7-(8-chloronaphthalen-1-yl)-2-(((S)-4-(1-(4-(3-(2,4-dihydroxy-5-isopropylphenyl)-5-hydroxy-4H-1,2,4-triazol-4-yl)benzyl)piperidine-4-carbonyl)-1-methylpiperazin-2-yl)methoxy)-5,6,7,8-tetrahydropyrido[3,4-d]pyrimidin-4-yl)piperazin-2-yl)acetonitrile, trifluoroacetic acid

¹H NMR (400 MHz, MeOD) δ 7.83 (d, J=7.8 Hz, 1H), 7.69 (dd, J=9.4, 4.5 Hz, 1H), 7.59-7.47 (m, 4H), 7.43-7.27 (m, 4H), 6.88-6.69 (m, 2H), 6.29 (d, J=16.8 Hz, 1H), 6.22 (s, 1H), 5.84 (d, J=10.5 Hz, 1H), 4.75-4.47 (m, 3H), 4.41-3.98 (m, 7H), 3.83-3.42 (m, 9H), 3.26-3.14 (m, 4H), 3.07 (m, 8H), 2.96-2.67 (m, 3H), 2.13-1.81 (m, 5H), 1.01 (d, J=6.2 Hz, 6H). LC-MS: m/z 1033.5 [M+H]⁺.

### Compound 203

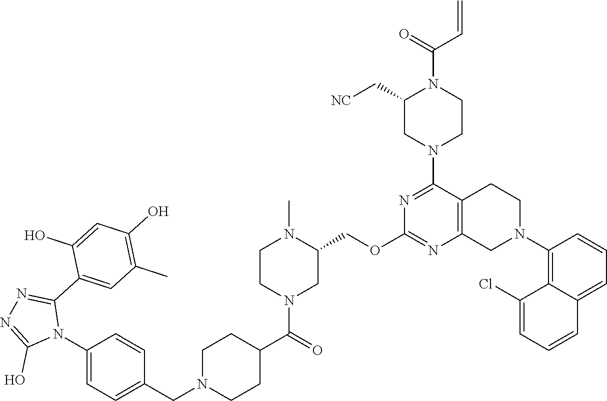 [SMILES: C=CC(=O)N1CCN(c2nc(OC[C@@H]3CN(C(=O)C4CCN(Cc5ccc(-n6c(O)nnc6-c6cc(C)c(O)cc6O)cc5)CC4)CCN3C)nc3c2CCN(c2cccc4cccc(Cl)c24)C3)C[C@@H]1CC#N]

### 2-((S)-1-acryloyl-4-(7-(8-chloronaphthalen-1-yl)-2-(((S)-4-(1-(4-(3-(2,4-dihydroxy-5-methylphenyl)-5-hydroxy-4H-1,2,4-triazol-4-yl)benzyl)piperidine-4-carbonyl)-1-methylpiperazin-2-yl)methoxy)-5,6,7,8-tetrahydropyrido[3,4-d]pyrimidin-4-yl)piperazin-2-yl)acetonitrile, trifluoroacetic acid

¹H NMR (400 MHz, MeOD) δ 7.84 (d, J=7.5 Hz, 1H), 7.71 (d, J=8.0 Hz, 1H), 7.55-7.47 (m, 4H), 7.41-7.32 (m, 4H), 6.92-6.78 (m, 2H), 6.30 (d, J=16.5 Hz, 1H), 6.18 (s, 1H), 5.84 (d, J=10.1 Hz, 1H), 4.70-4.63 (m, 2H), 4.40-4.28 (m, 5H), 4.23-4.09 (m, 2H), 3.77-3.69 (m, 2H), 3.67-3.57 (m, 5H), 3.55-3.46 (m, 3H), 3.26-3.17 (m, 4H), 3.13-3.01 (m, 8H), 2.96-2.88 (m, 2H), 2.79-2.72 (m, 1H), 2.06-1.98 (m, 7H). LC-MS: m/z 1007.4 [M+H]⁺.

### Compound 204

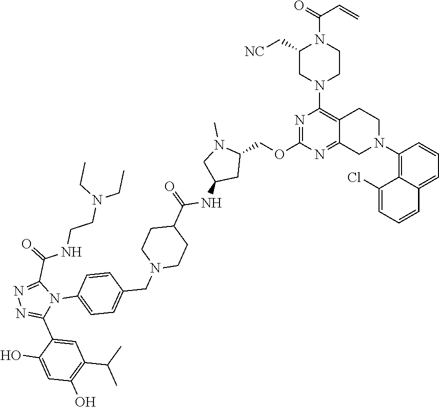 [SMILES: C=CC(=O)N1CCN(c2nc(OC[C@@H]3C[C@@H](NC(=O)C4CCN(Cc5ccc(-n6c(C(=O)NCCN(CC)CC)nnc6-c6cc(C(C)C)c(O)cc6O)cc5)CC4)CN3C)nc3c2CCN(c2cccc4cccc(Cl)c24)C3)C[C@@H]1CC#N]

### N-((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-1-(4-(3-((2-(diethylamino)ethyl)carbamoyl)-5-(2,4-dihydroxy-5-isopropylphenyl)-4H-1,2,4-triazol-4-yl)benzyl)piperidine-4-carboxamide

¹H NMR (400 MHz, MeOD) δ 7.82 (d, J=8.1 Hz, 1H), 7.67 (d, J=10.5 Hz, 1H), 7.53-7.42 (m, 4H), 7.38-7.29 (m, 2H), 7.25-7.22 (m, 2H), 6.89-6.70 (m, 2H), 6.33-6.23 (m, 2H), 5.82 (d, J=10.1 Hz, 1H), 4.37-4.29 (m, 4H), 4.20-4.07 (m, 2H), 3.74-3.67 (m, 1H), 3.54-3.52 (m, 2H), 3.18-3.13 (m, 8H), 3.03-2.99 (m, 1H), 2.95-2.90 (m, 3H), 2.47 (s, 3H), 2.21-2.02 (m, 5H), 1.90-1.83 (m, 8H), 1.78-1.74 (m, 1H), 0.90 (d, J=6.9 Hz, 6H). LC-MS: m/z 1061.5 [M+H]⁺.

### Compound 205

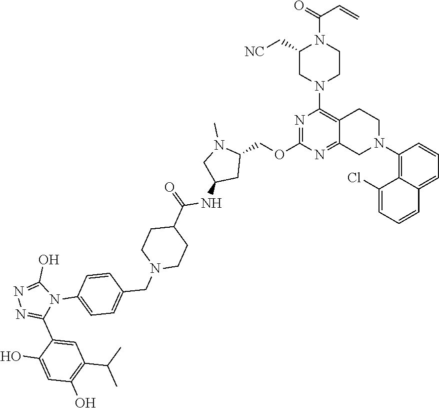 [SMILES: C=CC(=O)N1CCN(c2nc(OC[C@@H]3C[C@@H](NC(=O)C4CCN(Cc5ccc(-n6c(O)nnc6-c6cc(C(C)C)c(O)cc6O)cc5)CC4)CN3C)nc3c2CCN(c2cccc4cccc(Cl)c24)C3)C[C@@H]1CC#N]

### N-((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-1-(4-(3-(2,4-dihydroxy-5-isopropylphenyl)-5-hydroxy-4H-1,2,4-triazol-4-yl)benzyl)piperidine-4-carboxamide

¹H NMR (400 MHz, MeOD) δ 7.82 (d, J=8.1 Hz, 1H), 7.67 (d, J=10.4 Hz, 1H), 7.54-7.45 (m, 4H), 7.39-7.29 (m, 4H), 6.93-6.74 (m, 1H), 6.66 (s, 1H), 6.35-6.23 (m, 2H), 5.83 (d, J=12.0 Hz, 1H), 4.42-4.26 (m, 5H), 4.21-4.07 (m, 2H), 3.77-3.62 (m, 2H), 3.57-3.55 (m, 2H), 3.41-3.36 (m, 3H), 3.21-3.10 (m, 3H), 3.01-2.89 (m, 5H), 2.68-2.59 (m, 6H), 2.47 (s, 3H), 2.25-2.02 (m, 6H), 1.93-1.88 (m, 1H), 1.82-1.71 (m, 4H), 1.34-1.28 (m, 4H), 1.05 (t, J=7.2 Hz, 6H), 0.86 (d, J=6.9 Hz, 6H). LC-MS: m/z 1035.5 [M+H]⁺.

### Compound 206

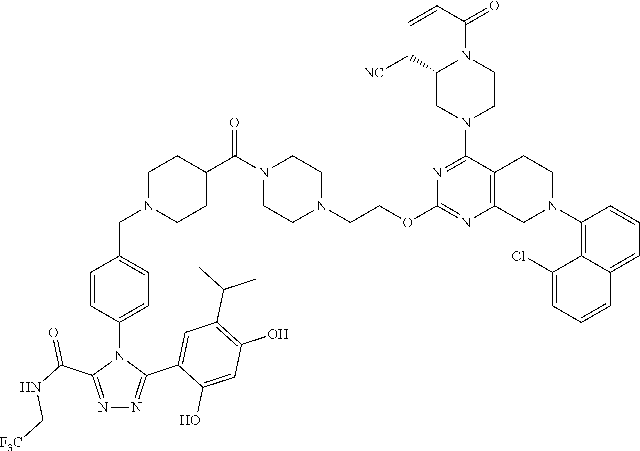 [SMILES: C=CC(=O)N1CCN(c2nc(OCCN3CCN(C(=O)C4CCN(Cc5ccc(-n6c(C(=O)NCC(F)(F)F)nnc6-c6cc(C(C)C)c(O)cc6O)cc5)CC4)CC3)nc3c2CCN(c2cccc4cccc(Cl)c24)C3)C[C@@H]1CC#N]

### (S)-4-(4-((4-(4-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperazine-1-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide, trifluoroacetic acid

¹H NMR (400 MHz, MeOD) δ 7.84 (d, J=7.5 Hz, 1H), 7.72 (d, J=8.3 Hz, 1H), 7.62 (d, J=8.4 Hz, 2H), 7.56-7.46 (m, 4H), 7.44-7.30 (m, 2H), 6.92-6.67 (m, 2H), 6.30 (d, J=16.1 Hz, 2H), 5.84 (d, J=11.1 Hz, 1H), 4.47-4.24 (m, 5H), 4.21-4.04 (m, 1H), 4.04-3.72 (m, 8H), 3.69-3.57 (m, 5H), 3.56-3.38 (m, 7H), 3.27-3.18 (m, 2H), 3.16-2.91 (m, 6H), 2.90-2.66 (m, 2H), 2.13-1.77 (m, 5H), 0.93 (d, J=6.9 Hz, 6H). LC-MS: m/z 1144.5 [M+H]⁺.

### Compound 207

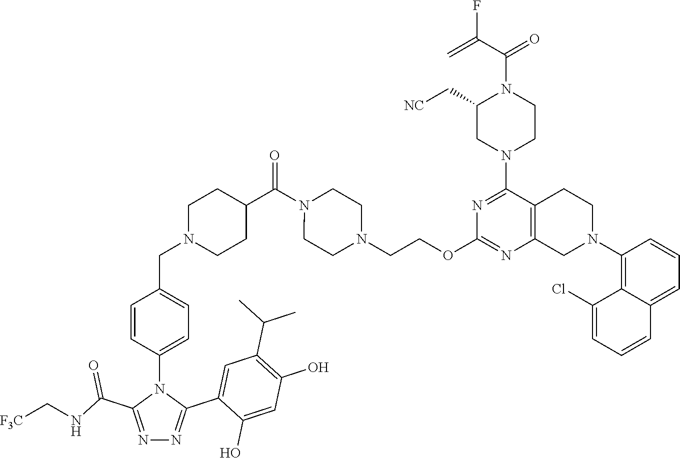 [SMILES: C=C(F)C(=O)N1CCN(c2nc(OCCN3CCN(C(=O)C4CCN(Cc5ccc(-n6c(C(=O)NCC(F)(F)F)nnc6-c6cc(C(C)C)c(O)cc6O)cc5)CC4)CC3)nc3c2CCN(c2cccc4cccc(Cl)c24)C3)C[C@@H]1CC#N]

### (S)-4-(4-((4-(4-(2-((7-(8-chloronaphthalen-1-yl)-4-(3-(cyanomethyl)-4-(2-fluoroacryloyl)piperazin-1-yl)-5,6,7,8-tetrahydropyrido[3,4-d]pyrimidin-2-yl)oxy)ethyl)piperazine-1-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide

¹H NMR (400 MHz, MeOD) δ 7.84 (d, J=8.2 Hz, 1H), 7.70 (d, J=7.8 Hz, 1H), 7.61 (d, J=8.4 Hz, 2H), 7.55-7.47 (m, 5H), 7.40-7.33 (m, 2H), 6.75 (s, 1H), 6.27 (s, 1H), 5.39-5.28 (m, 2H), 4.40-4.20 (m, 6H), 4.01-3.95 (m, 3H), 3.65-3.58 (m, 5H), 3.49-3.41 (m, 5H), 3.15-2.96 (m, 8H), 2.05-1.93 (m, 4H), 1.71-1.60 (m, 4H), 1.07-1.00 (m, 4H), 0.94 (d, J=6.9 Hz, 6H). LC-MS: m/z 1163.9 [M+H]⁺.

### Compound 208

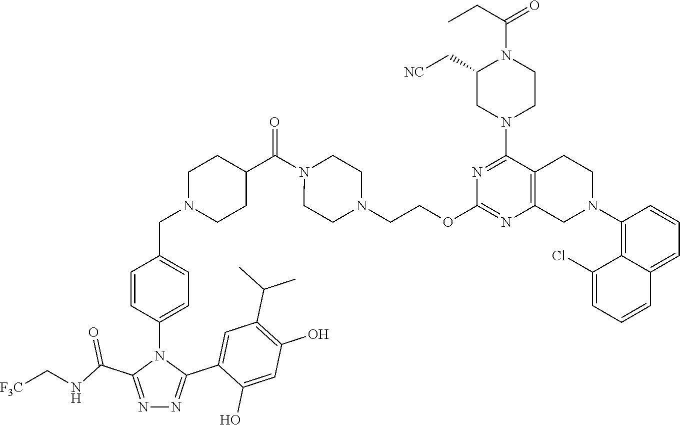 [SMILES: CCC(=O)N1CCN(c2nc(OCCN3CCN(C(=O)C4CCN(Cc5ccc(-n6c(C(=O)NCC(F)(F)F)nnc6-c6cc(C(C)C)c(O)cc6O)cc5)CC4)CC3)nc3c2CCN(c2cccc4cccc(Cl)c24)C3)C[C@@H]1CC#N]

### (S)-4-(4-((4-(4-(2-((7-(8-chloronaphthalen-1-yl)-4-(3-(cyanomethyl)-4-propionylpiperazin-1-yl)-5,6,7,8-tetrahydropyrido[3,4-d]pyrimidin-2-yl)oxy)ethyl)piperazine-1-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide

¹H NMR (400 MHz, MeOD) δ 7.82 (d, J=8.2 Hz, 1H), 7.67 (d, J=8.1 Hz, 1H), 7.54-7.45 (m, 4H), 7.38-7.28 (m, 4H), 6.68 (s, 1H), 6.30 (s, 1H), 4.53-4.48 (m, 2H), 4.34-4.27 (m, 1H), 4.18-4.13 (m, 1H), 4.02-3.92 (m, 3H), 3.71-3.48 (m, 9H), 3.23-3.10 (m, 3H), 3.04-2.93 (m, 4H), 2.87-2.78 (m, 3H), 2.72-2.47 (m, 8H), 2.16-2.08 (m, 2H), 2.03 (s, 3H), 1.82-1.66 (m, 4H), 1.19-1.13 (m, 3H), 0.86 (d, J=6.9 Hz, 6H). LC-MS: m/z 1146.5 [M+H]⁺.

### Compound 209

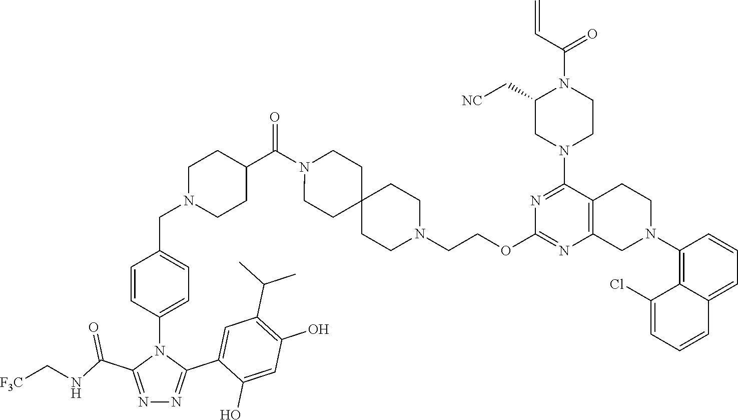 [SMILES: C=CC(=O)N1CCN(c2nc(OCCN3CCC4(CC3)CCN(C(=O)C3CCN(Cc5ccc(-n6c(C(=O)NCC(F)(F)F)nnc6-c6cc(C(C)C)c(O)cc6O)cc5)CC3)CC4)nc3c2CCN(c2cccc4cccc(Cl)c24)C3)C[C@@H]1CC#N]

### (S)-4-(4-((4-(9-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)-3,9-diazaspiro[5.5]undecane-3-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide

¹H NMR (400 MHz, MeOD) δ 7.84 (d, J=7.6 Hz, 1H), 7.71 (d, J=8.4 Hz, 1H), 7.65-7.60 (m, 2H), 7.56-7.47 (m, 4H), 7.42-7.28 (m, 2H), 6.90-6.72 (m, 2H), 6.30 (d, J=18.2 Hz, 2H), 5.84 (d, J=10.2 Hz, 1H), 4.81-4.70 (m, 3H), 4.42-4.20 (m, 5H), 4.03-3.95 (m, 2H), 3.87-3.67 (m, 2H), 3.65-3.46 (m, 12H), 3.27-3.17 (m, 3H), 3.15-2.87 (m, 7H), 2.81-2.69 (m, 1H), 2.06-1.89 (m, 6H), 1.83-1.61 (m, 5H), 1.58-1.40 (m, 2H), 1.32-1.26 (m, 1H), 0.94 (d, J=6.9 Hz, 6H). LC-MS: m/z 1210.7 [M+H]⁺.

### Compound 210

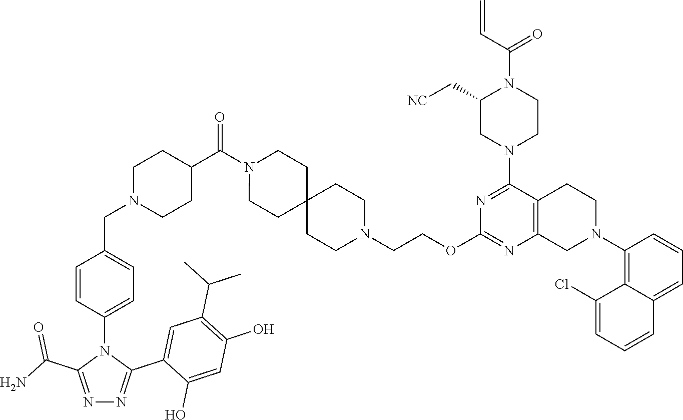 [SMILES: C=CC(=O)N1CCN(c2nc(OCCN3CCC4(CC3)CCN(C(=O)C3CCN(Cc5ccc(-n6c(C(N)=O)nnc6-c6cc(C(C)C)c(O)cc6O)cc5)CC3)CC4)nc3c2CCN(c2cccc4cccc(Cl)c24)C3)C[C@@H]1CC#N]

### (S)-4-(4-((4-(9-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)-3,9-diazaspiro[5.5]undecane-3-carbonyl)piperidin-1-yl)methyl)phenyl)-5-(2,4-dihydroxy-5-isopropylphenyl)-4H-1,2,4-triazole-3-carboxamide

¹H NMR (400 MHz, MeOD) δ 7.85 (d, J=8.1 Hz, 1H), 7.73 (d, J=8.2 Hz, 1H), 7.62 (d, J=8.3 Hz, 2H), 7.57-7.46 (m, 4H), 7.44-7.31 (m, 2H), 6.89-6.69 (m, 2H), 6.36-6.21 (m, 2H), 5.85 (d, J=10.7 Hz, 1H), 4.53 (d, J=14.7 Hz, 1H), 4.45-4.27 (m, 4H), 4.20-4.05 (m, 1H), 3.90-3.77 (m, 2H), 3.71-3.44 (m, 14H), 3.27-3.19 (m, 3H), 3.15-2.92 (m, 6H), 2.86-2.73 (m, 1H), 2.01-1.83 (m, 6H), 1.83-1.56 (m, 5H), 1.55-1.38 (m, 2H), 1.35-1.22 (m, 1H), 0.93 (d, J=6.9 Hz, 6H). LC-MS: m/z 1130.5 [M+H]⁺.

### Compound 211

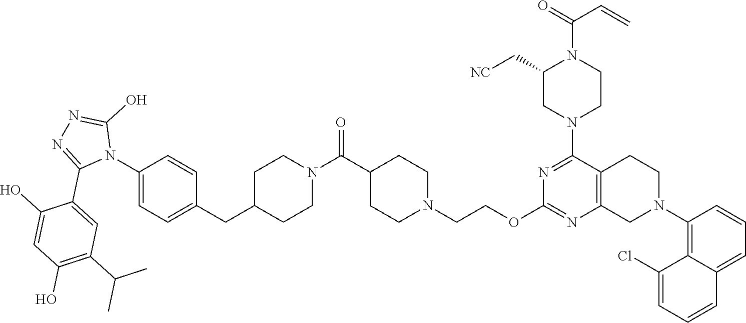 [SMILES: C=CC(=O)N1CCN(c2nc(OCCN3CCC(C(=O)N4CCC(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(4-(4-(3-(2,4-dihydroxy-5-isopropylphenyl)-5-hydroxy-4H-1,2,4-triazol-4-yl)benzyl)piperidine-1-carbonyl)piperidin-1-yl)ethoxy)-5,6,7,8-tetrahydropyrido[3,4-d]pyrimidin-4-yl)piperazin-2-yl)acetonitrile

¹H NMR (400 MHz, MeOD) δ 8.45 (br, 5H), 7.83 (d, J=8.8 Hz, 1H), 7.68 (s, 1H), 7.53 (d, J=7.5 Hz, 2H), 7.40-7.18 (m, 7H), 6.69 (s, 1H), 6.28 (s, 2H), 4.58 (m, 4H), 4.53-4.48 (m, 3H), 4.19 (m, 2H), 4.05-3.98 (m, 2H), 3.72 (m, 2H), 3.48 (m, 8H), 3.07-3.00 (m, 4H), 2.61 (m, 3H), 2.23-2.15 (m, 2H), 2.03 (m, 5H), 1.89 (m, 8H), 0.89 (d, J=6.9 Hz, 6H). LC-MS: m/z 1034.5 [M+H]⁺.

### Compound 212

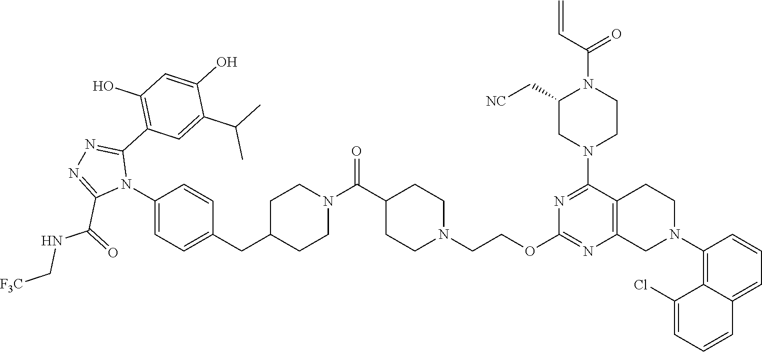 [SMILES: C=CC(=O)N1CCN(c2nc(OCCN3CCC(C(=O)N4CCC(Cc5ccc(-n6c(C(=O)NCC(F)(F)F)nnc6-c6cc(C(C)C)c(O)cc6O)cc5)CC4)CC3)nc3c2CCN(c2cccc4cccc(Cl)c24)C3)C[C@@H]1CC#N]

### (S)-4-(4-((1-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperidin-4-yl)methyl)phenyl)-5-(2,4-dihydroxy-5-isopropylphenyl)-N-(2,2,2-trifluoroethyl)-4H-1,2,4-triazole-3-carboxamide

¹H NMR (400 MHz, MeOD) δ 7.81 (d, J=8.1 Hz, 1H), 7.67 (d, J=8.2 Hz, 1H), 7.59-7.46 (m, 2H), 7.32 (m, 7H), 6.81 (s, 1H), 6.66 (s, 1H), 6.38-6.23 (m, 2H), 5.83 (m, 1H), 4.50 (m, 4H), 4.31 (m, 2H), 4.04 (m, 7H), 3.73 (m, 1H), 3.59 (m, 1H), 3.19 (m, 3H), 3.14-3.04 (m, 5H), 2.99-2.87 (m, 2H), 2.81 (m, 2H), 2.68 (m, 5H), 2.56 (m, 1H), 2.25 (m, 2H), 1.76 (m, 9H), 0.86 (d, J=6.9 Hz, 6H). LC-MS: m/z 1143.5 [M+H]⁺.

### Compound 213

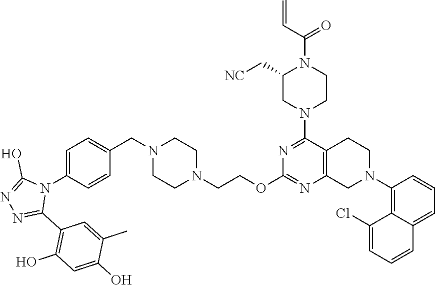 [SMILES: C=CC(=O)N1CCN(c2nc(OCCN3CCN(Cc4ccc(-n5c(O)nnc5-c5cc(C)c(O)cc5O)cc4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(4-(3-(2,4-dihydroxy-5-methylphenyl)-5-hydroxy-4H-1,2,4-triazol-4-yl)benzyl)piperazin-1-yl)ethoxy)-5,6,7,8-tetrahydropyrido[3,4-d]pyrimidin-4-yl)piperazin-2-yl)acetonitrile

¹H NMR (400 MHz, MeOD) δ 7.85 (d, J=8.2 Hz, 1H), 7.73 (d, J=8.2 Hz, 1H), 7.51 (dd, J=11.7, 7.4 Hz, 4H), 7.37 (dd, J=10.6, 8.6 Hz, 4H), 6.90-6.73 (m, 2H), 6.37-6.11 (m, 2H), 5.85 (d, J=10.5 Hz, 1H), 4.72 (dd, J=11.5, 5.7 Hz, 2H), 4.34 (d, J=16.1 Hz, 3H), 4.12 (d, J=3.9 Hz, 1H), 3.84 (dd, J=18.4, 9.2 Hz, 3H), 3.61 (dd, J=12.6, 2.6 Hz, 3H), 3.52-3.46 (m, 3H), 3.40-3.37 (m, 3H), 3.27-3.22 (m, 2H), 3.13 (t, J=13.1 Hz, 8H), 2.79 (dd, J=8.0, 7.1 Hz, 1H), 1.97 (s, 2H). LC-MS: m/z 896.8 [M+H]⁺.

### Compound 214

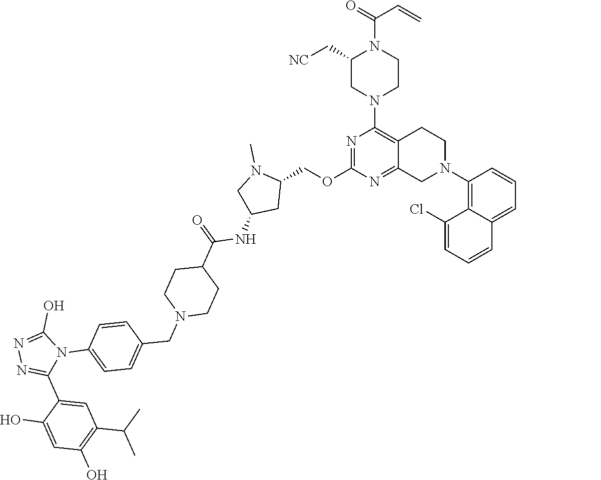 [SMILES: C=CC(=O)N1CCN(c2nc(OC[C@@H]3C[C@H](NC(=O)C4CCN(Cc5ccc(-n6c(O)nnc6-c6cc(C(C)C)c(O)cc6O)cc5)CC4)CN3C)nc3c2CCN(c2cccc4cccc(Cl)c24)C3)C[C@@H]1CC#N]

### N-((3S,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-1-(4-(3-(2,4-dihydroxy-5-isopropylphenyl)-5-hydroxy-4H-1,2,4-triazol-4-yl)benzyl)piperidine-4-carboxamide

¹H NMR (400 MHz, MeOD) δ 7.79 (d, J=8.3 Hz, 1H), 7.66 (d, J=8.1 Hz, 1H), 7.54-7.46 (m, 2H), 7.37-7.31 (m, 2H), 7.23 (dd, J=8.4, 2.1 Hz, 2H), 7.17 (d, J=7.9 Hz, 2H), 6.80 (s, 1H), 6.70 (s, 1H), 6.29 (d, J=13.2 Hz, 2H), 5.83 (m, 1H), 4.58-4.43 (m, 2H), 4.40-4.23 (m, 4H), 4.13 (m, 2H), 3.68 (m, 3H), 3.17 (m, 6H), 3.02-2.98 (m, 1H), 2.92-2.86 (m, 2H), 2.75 (m, 4H), 2.56 (m, 1H), 2.43 (m, 3H), 2.28 (m, 1H), 1.92-1.83 (m, 3H), 1.71 (m, 5H), 1.30 (m, 1H), 0.90 (d, J=6.9 Hz, 6H). LC-MS: m/z 1035.4 [M+H]⁺.

### Compound 215

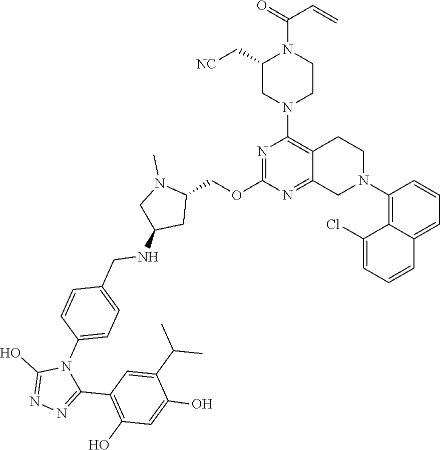 [SMILES: C=CC(=O)N1CCN(c2nc(OC[C@@H]3C[C@@H](NCc4ccc(-n5c(O)nnc5-c5cc(C(C)C)c(O)cc5O)cc4)CN3C)nc3c2CCN(c2cccc4cccc(Cl)c24)C3)C[C@@H]1CC#N]

### 2-((S)-1-acryloyl-4-(7-(8-chloronaphthalen-1-yl)-2-(((2S,4R)-4-((4-(3-(2,4-dihydroxy-5-isopropylphenyl)-5-hydroxy-4H-1,2,4-triazol-4-yl)benzyl)amino)-1-methylpyrrolidin-2-yl)methoxy)-5,6,7,8-tetrahydropyrido[3,4-d]pyrimidin-4-yl)piperazin-2-yl)acetonitrile

¹H NMR (400 MHz, MeOD) δ 7.84 (d, J=8.1 Hz, 1H), 7.71 (d, J=8.3 Hz, 1H), 7.63-7.47 (m, 5H), 7.37 (dt, J=6.9, 3.3 Hz, 4H), 6.89 (s, 1H), 6.75 (s, 1H), 6.30 (d, J=17.2 Hz, 1H), 6.19 (s, 1H), 5.84 (d, J=10.6 Hz, 1H), 4.79-4.75 (m, 1H), 4.62 (m, 2H), 4.32 (m, 5H), 4.03 (m, 4H), 3.76 (m, 1H), 3.63 (m, 1H), 3.22 (m, 1H), 3.16-2.98 (m, 6H), 2.93 (m, 1H), 2.78 (m, 1H), 2.54 (m, 2H), 1.02 (m, 7H). LC-MS: m/z 924.3 [M+H]⁺.

### Compound 216

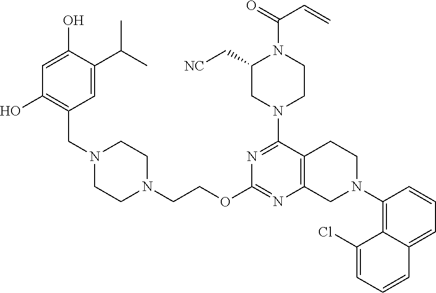 [SMILES: C=CC(=O)N1CCN(c2nc(OCCN3CCN(Cc4cc(C(C)C)c(O)cc4O)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(2,4-dihydroxy-5-isopropylbenzyl)piperazin-1-yl)ethoxy)-5,6,7,8-tetrahydropyrido[3,4-d]pyrimidin-4-yl)piperazin-2-yl)acetonitrile

¹H NMR (400 MHz, MeOD) δ 7.82 (d, J=8.2 Hz, 1H), 7.67 (d, J=8.0 Hz, 1H), 7.54-7.45 (m, 2H), 7.39-7.28 (m, 2H), 6.90-6.69 (m, 2H), 6.35-6.18 (m, 2H), 5.82 (d, J=10.8 Hz, 1H), 4.49 (t, J=5.4 Hz, 2H), 4.36-4.01 (m, 4H), 3.75-3.46 (m, 5H), 3.25-3.01 (m, 6H), 2.95-2.77 (m, 4H), 2.75-2.52 (m, 7H), 1.34-1.26 (m, 2H), 1.15 (d, J=6.9 Hz, 6H). LC-MS: m/z 765.4 [M+H]⁺.

### Compound 217

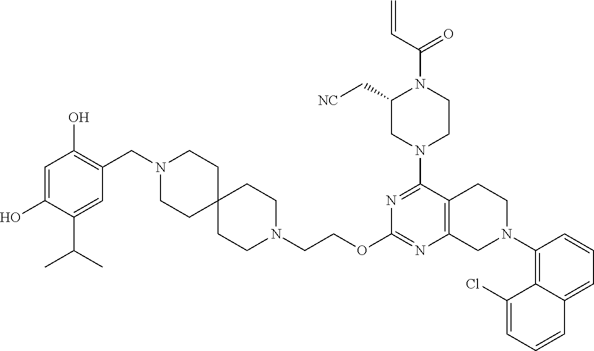 [SMILES: C=CC(=O)N1CCN(c2nc(OCCN3CCC4(CC3)CCN(Cc3cc(C(C)C)c(O)cc3O)CC4)nc3c2CCN(c2cccc4cccc(Cl)c24)C3)C[C@@H]1CC#N]

### (S)-2-(1-acryloyl-4-(7-(8-chloronaphthalen-1-yl)-2-(2-(9-(2,4-dihydroxy-5-isopropylbenzyl)-3,9-diazaspiro[5.5]undecan-3-yl)ethoxy)-5,6,7,8-tetrahydropyrido[3,4-d]pyrimidin-4-yl)piperazin-2-yl)acetonitrile

¹H NMR (400 MHz, MeOD) δ 7.82 (d, J=7.8 Hz, 1H), 7.70-7.64 (m, 1H), 7.54-7.45 (m, 2H), 7.40-7.28 (m, 2H), 6.94-6.72 (m, 2H), 6.28 (d, J=17.0 Hz, 1H), 6.22 (s, 1H), 5.83 (d, J=10.0 Hz, 1H), 4.50 (t, J=5.6 Hz, 2H), 4.33-4.27 (m, 1H), 4.23-3.98 (m, 3H), 3.76-3.53 (m, 5H), 3.51-3.40 (m, 1H), 3.24-3.04 (m, 6H), 2.94-2.87 (m, 1H), 2.82 (t, J=5.6 Hz, 2H), 2.75-2.55 (m, 9H), 1.32-1.26 (m, 8H), 1.15 (d, J=6.9 Hz, 6H). LC-MS: m/z 833.2 [M+H]⁺.

### Compound 218

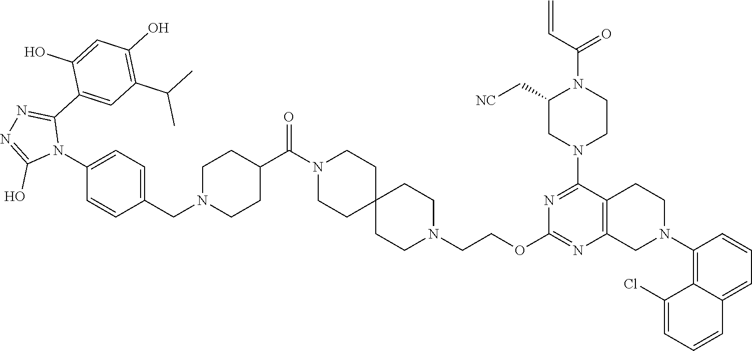 [SMILES: C=CC(=O)N1CCN(c2nc(OCCN3CCC4(CC3)CCN(C(=O)C3CCN(Cc5ccc(-n6c(O)nnc6-c6cc(C(C)C)c(O)cc6O)cc5)CC3)CC4)nc3c2CCN(c2cccc4cccc(Cl)c24)C3)C[C@@H]1CC#N]

### (S)-2-(1-acryloyl-4-(7-(8-chloronaphthalen-1-yl)-2-(2-(9-(1-(4-(3-(2,4-dihydroxy-5-isopropylphenyl)-5-hydroxy-4H-1,2,4-triazol-4-yl)benzyl)piperidine-4-carbonyl)-3,9-diazaspiro[5.5]undecan-3-yl)ethoxy)-5,6,7,8-tetrahydropyrido[3,4-d]pyrimidin-4-yl)piperazin-2-yl)acetonitrile

¹H NMR (400 MHz, MeOD) δ 7.84 (d, J=8.1 Hz, 1H), 7.70 (dd, J=7.7, 2.5 Hz, 1H), 7.58-7.49 (m, 4H), 7.42-7.32 (m, 4H), 6.88-6.78 (m, 2H), 6.29 (d, J=16.9 Hz, 1H), 6.22 (s, 1H), 5.84 (d, J=10.6 Hz, 1H), 4.77-4.66 (m, 3H), 4.38-4.20 (m, 5H), 4.17-4.10 (m, 1H), 3.74 (t, J=17.6 Hz, 1H), 3.65-3.49 (m, 12H), 3.28-3.16 (m, 5H), 3.14-2.86 (m, 8H), 2.80-2.68 (m, 1H), 2.01-1.87 (m, 6H), 1.36-1.24 (m, 5H), 1.01 (d, J=6.9 Hz, 6H). LC-MS: m/z 1104.0 [M+H]⁺.

### Compound 219

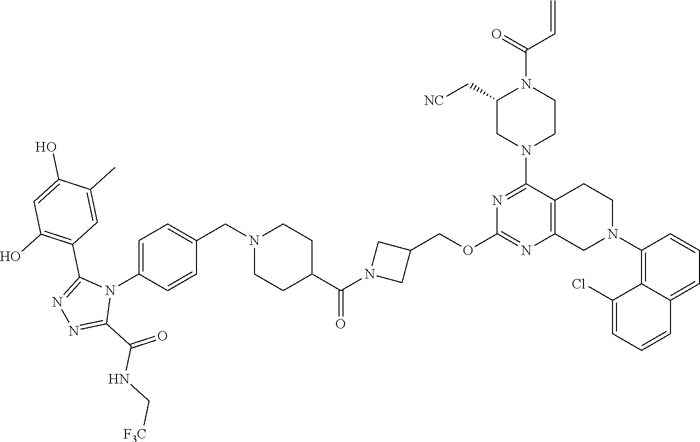 [SMILES: C=CC(=O)N1CCN(c2nc(OCC3CN(C(=O)C4CCN(Cc5ccc(-n6c(C(=O)NCC(F)(F)F)nnc6-c6cc(C)c(O)cc6O)cc5)CC4)C3)nc3c2CCN(c2cccc4cccc(Cl)c24)C3)C[C@@H]1CC#N]

### (S)-4-(4-((4-(3-(((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)azetidine-1-carbonyl)piperidin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) δ 7.84 (d, J=8.2 Hz, 1H), 7.70 (d, J=15.9 Hz, 1H), 7.62-7.48 (m, 4H), 7.47-7.30 (m, 4H), 6.90-6.71 (m, 2H), 6.31 (d, J=16.6 Hz, 1H), 6.22 (s, 1H), 5.85 (d, J=10.3 Hz, 1H), 4.75-4.62 (m, 2H), 4.61-4.49 (m, 1H), 4.48-4.29 (m, 5H), 4.26-4.08 (m, 3H), 4.03-3.94 (m, 2H), 3.93-3.67 (m, 3H), 3.67-3.41 (m, 5H), 3.27-2.58 (m, 9H), 2.09-1.79 (m, 7H), 1.45-1.14 (m, 1H). LC-MS: m/z 1074.0 [M+H]⁺.

### Compound 220

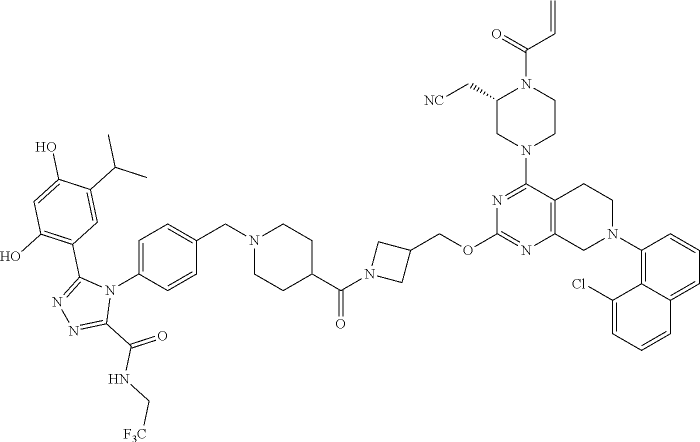 [SMILES: C=CC(=O)N1CCN(c2nc(OCC3CN(C(=O)C4CCN(Cc5ccc(-n6c(C(=O)NCC(F)(F)F)nnc6-c6cc(C(C)C)c(O)cc6O)cc5)CC4)C3)nc3c2CCN(c2cccc4cccc(Cl)c24)C3)C[C@@H]1CC#N]

### (S)-4-(4-((4-(3-(((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)azetidine-1-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide

¹H NMR (400 MHz, MeOD) δ 7.84 (d, J=8.3 Hz, 1H), 7.72 (d, J=8.0 Hz, 1H), 7.60 (d, J=8.3 Hz, 2H), 7.57-7.44 (m, 4H), 7.41-7.34 (m, 2H), 6.75 (s, 2H), 6.37-6.17 (m, 2H), 5.84 (d, J=10.9 Hz, 1H), 5.04 (d, J=36.6 Hz, 1H), 4.64 (d, J=5.3 Hz, 2H), 4.51-4.28 (m, 6H), 4.15 (dd, J=18.8, 9.2 Hz, 3H), 3.98 (q, J=9.2 Hz, 2H), 3.93-3.69 (m, 3H), 3.59 (dt, J=41.9, 16.1 Hz, 4H), 3.25-2.92 (m, 7H), 2.82-2.60 (m, 2H), 2.05-1.83 (m, 4H), 1.32 (dd, J=13.3, 5.6 Hz, 2H), 0.93 (d, J=6.9 Hz, 6H). LC-MS: m/z 1102.0 [M+H]⁺.

### Compound 221

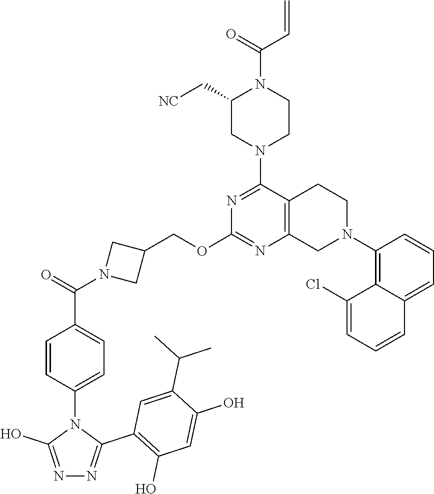 [SMILES: C=CC(=O)N1CCN(c2nc(OCC3CN(C(=O)c4ccc(-n5c(O)nnc5-c5cc(C(C)C)c(O)cc5O)cc4)C3)nc3c2CCN(c2cccc4cccc(Cl)c24)C3)C[C@@H]1CC#N]

### (S)-2-(1-acryloyl-4-(7-(8-chloronaphthalen-1-yl)-2-((1-(4-(3-(2,4-dihydroxy-5-isopropylphenyl)-5-hydroxy-4H-1,2,4-triazol-4-yl)benzoyl)azetidin-3-yl)methoxy)-5,6,7,8-tetrahydropyrido[3,4-d]pyrimidin-4-yl)piperazin-2-yl)acetonitrile

¹H NMR (400 MHz, MeOD) δ 7.84 (d, J=8.3 Hz, 1H), 7.72 (d, J=8.0 Hz, 1H), 7.60 (d, J=8.3 Hz, 2H), 7.57-7.44 (m, 4H), 7.41-7.34 (m, 2H), 6.93-6.58 (m, 2H), 6.37-6.17 (m, 2H), 5.84 (d, J=10.9 Hz, 1H), 5.12-4.90 (m, 1H), 4.64 (d, J=5.3 Hz, 2H), 4.51-4.28 (m, 6H), 4.26-4.06 (m, 3H), 4.02-3.94 (m, 2H), 3.93-3.69 (m, 3H), 3.64-3.48 (m, 4H), 3.25-2.92 (m, 7H), 2.82-2.60 (m, 2H), 2.05-1.83 (m, 4H), 1.35-1.28 (m, 2H), 0.93 (d, J=6.9 Hz, 6H). LC-MS: m/z 896.0 [M+H]⁺.

### Compound 222

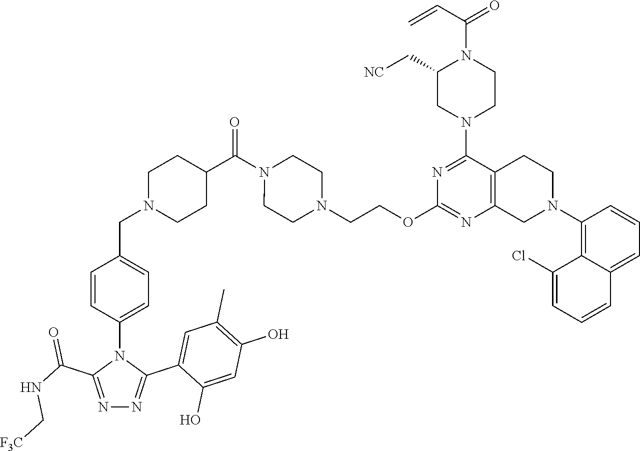 [SMILES: C=CC(=O)N1CCN(c2nc(OCCN3CCN(C(=O)C4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@@H]1CC#N]

### (S)-4-(4-((4-(4-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperazine-1-carbonyl)piperidin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) δ 7.84 (d, J=7.9 Hz, 1H), 7.70 (d, J=8.7 Hz, 1H), 7.59-7.48 (m, 4H), 7.48-7.30 (m, 4H), 6.89-6.74 (m, 2H), 6.29 (d, J=17.2 Hz, 1H), 6.22 (s, 1H), 5.84 (d, J=10.3 Hz, 1H), 4.79-4.72 (m, 2H), 4.41-4.23 (m, 4H), 4.04-3.86 (m, 6H), 3.80-3.54 (m, 7H), 3.52-3.38 (m, 6H), 3.25-3.16 (m, 2H), 3.14-2.99 (m, 4H), 2.98-2.87 (m, 2H), 2.85-2.65 (m, 1H), 2.06-1.92 (m, 7H), 1.33-1.27 (m, 2H). LC-MS: m/z 1116.2 [M+H]⁺.

### Compound 223

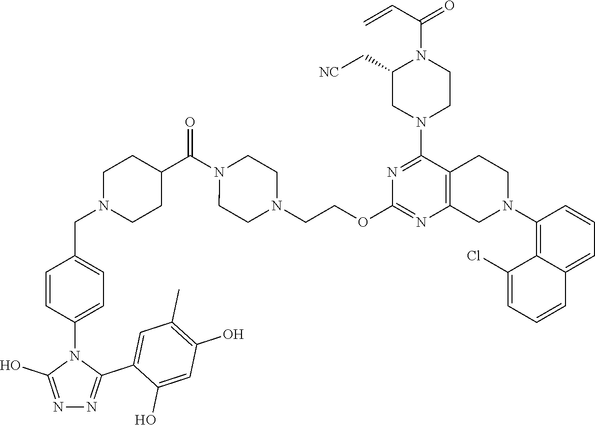 [SMILES: C=CC(=O)N1CCN(c2nc(OCCN3CCN(C(=O)C4CCN(Cc5ccc(-n6c(O)nnc6-c6cc(C)c(O)cc6O)cc5)CC4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(1-(4-(3-(2,4-dihydroxy-5-methylphenyl)-5-hydroxy-4H-1,2,4-triazol-4-yl)benzyl)piperidine-4-carbonyl)piperazin-1-yl)ethoxy)-5,6,7,8-tetrahydropyrido[3,4-d]pyrimidin-4-yl)piperazin-2-yl)acetonitrile

¹H NMR (400 MHz, MeOD) δ 7.84 (d, J=7.4 Hz, 1H), 7.71 (d, J=8.5 Hz, 1H), 7.56-7.49 (m, 4H), 7.41-7.32 (m, 4H), 6.91-6.74 (m, 2H), 6.30 (d, J=17.2 Hz, 1H), 6.18 (s, 1H), 5.84 (d, J=10.5 Hz, 1H), 5.11-4.96 (m, 2H), 4.41-4.11 (m, 6H), 4.02-3.85 (m, 4H), 3.83-3.61 (m, 6H), 3.56-3.39 (m, 9H), 3.26-3.16 (m, 2H), 3.14-2.64 (m, 7H), 2.04-1.89 (m, 7H), 1.33-1.28 (m, 1H). LC-MS: m/z 1107.4 [M+H]⁺.

### Compound 224

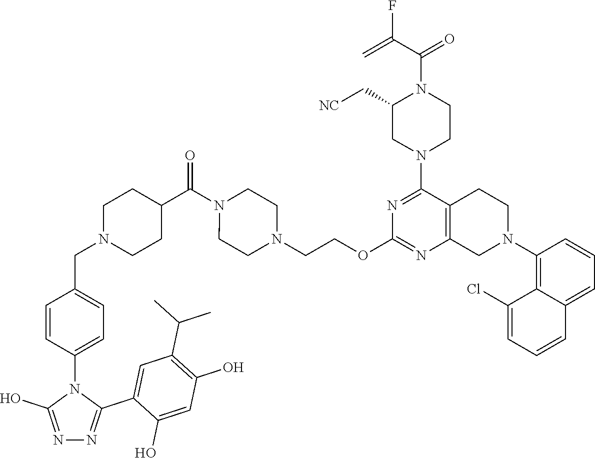 [SMILES: C=C(F)C(=O)N1CCN(c2nc(OCCN3CCN(C(=O)C4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(4-(7-(8-chloronaphthalen-1-yl)-2-(2-(4-(1-(4-(3-(2,4-dihydroxy-5-isopropylphenyl)-5-hydroxy-4H-1,2,4-triazol-4-yl)benzyl)piperidine-4-carbonyl)piperazin-1-yl)ethoxy)-5,6,7,8-tetrahydropyrido[3,4-d]pyrimidin-4-yl)-1-(2-fluoroacryloyl)piperazin-2-yl)acetonitrile

¹H NMR (400 MHz, MeOD) δ 7.86-7.82 (m, 1H), 7.71 (d, J=8.2 Hz, 1H), 7.58-7.48 (m, 4H), 7.41-7.32 (m, 4H), 6.86 (s, 1H), 6.22 (s, 1H), 5.42-5.20 (m, 3H), 4.83-4.77 (m, 3H), 4.44-4.18 (m, 6H), 4.00-3.86 (m, 4H), 3.68-3.52 (m, 9H), 3.28-2.96 (m, 9H), 2.04-1.90 (m, 4H), 1.40-1.27 (m, 3H), 1.00 (d, J=6.9 Hz, 6H). LC-MS: m/z 1053.5 [M+H]⁺.

### Compound 225

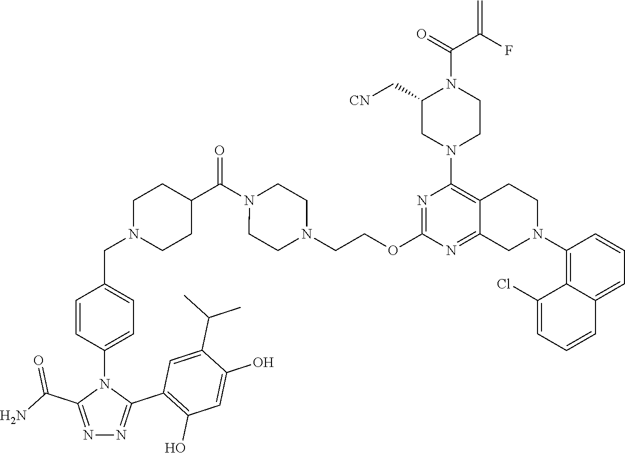

### (R)-4-(4-((4-(4-(2-((7-(8-chloronaphthalen-1-yl)-4-(4-(2-fluoroacryloyl)-3-(isocyanomethyl)piperazin-1-yl)-5,6,7,8-tetrahydropyrido[3,4-d]pyrimidin-2-yl)oxy)ethyl)piperazine-1-carbonyl)piperidin-1-yl)methyl)phenyl)-5-(2,4-dihydroxy-5-isopropylphenyl)-4H-1,2,4-triazole-3-carboxamide

¹H NMR (400 MHz, MeOD) δ 7.84 (d, J=7.4 Hz, 1H), 7.70 (d, J=8.4 Hz, 1H), 7.62-7.58 (m, 2H), 7.54-7.47 (m, 4H), 7.40-7.31 (m, 2H), 6.74 (s, 1H), 6.27 (s, 1H), 5.40-5.27 (m, 2H), 4.78-4.73 (m, 2H), 4.54-4.16 (m, 6H), 3.98-3.86 (m, 3H), 3.79-3.57 (m, 7H), 3.53-3.38 (m, 6H), 3.24-2.97 (m, 8H), 2.06-1.91 (m, 4H), 1.35-1.26 (m, 3H), 0.93 (d, J=6.9 Hz, 6H). LC-MS: m/z 1081.0 [M+H]⁺.

### Compound 226

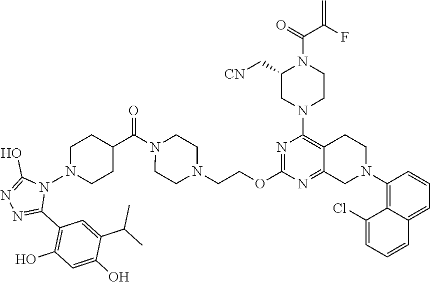

### (R)-1-(4-(7-(8-chloronaphthalen-1-yl)-2-(2-(4-(4-(3-(2,4-dihydroxy-5-isopropylphenyl)-5-hydroxy-4H-1,2,4-triazol-4-yl)benzoyl)piperazin-1-yl)ethoxy)-5,6,7,8-tetrahydropyrido[3,4-d]pyrimidin-4-yl)-2-(isocyanomethyl)piperazin-1-yl)-2-fluoroprop-2-en-1-one

¹H NMR (400 MHz, MeOD) δ 7.84 (d, J=8.3 Hz, 1H), 7.71 (d, J=8.4 Hz, 1H), 7.56-7.50 (m, 4H), 7.40-7.32 (m, 4H), 6.90 (s, 1H), 6.21 (s, 1H), 5.44-5.23 (m, 3H), 4.84-4.76 (m, 3H), 4.40-4.21 (m, 3H), 3.81-3.60 (m, 7H), 3.54-3.43 (m, 6H), 3.25-2.92 (m, 5H), 1.34-1.28 (m, 3H), 1.02 (d, J=6.9 Hz, 6H). LC-MS: m/z 956.0 [M+H]⁺.

### Compound 227

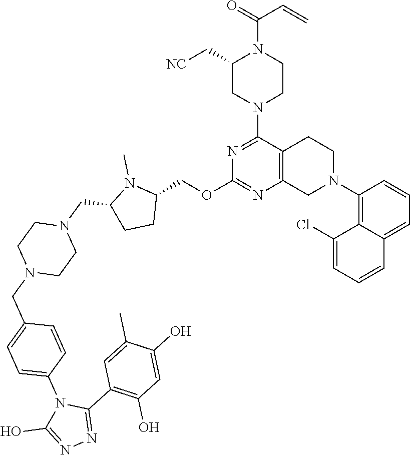 [SMILES: C=CC(=O)N1CCN(c2nc(OC[C@@H]3CC[C@H](CN4CCN(Cc5ccc(-n6c(O)nnc6-c6cc(C)c(O)cc6O)cc5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-{[4-({4-[3-(2,4-dihydroxy-5-methylphenyl)-5-hydroxy-1,2,4-triazol-4-yl]phenyl}methyl)piperazin-1-yl]meth yl}-1-methylpyrrolidin-2-yl]methoxy}-5H,6H,8H-pyrido[3,4-d] pyrimidin-4-yl]-1-(prop-2-enoyl)piperazin-2-yl]acetonitrile

¹H NMR (DMSO-d₆, 400 MHz): δ 11.88 (s, 1H), 9.55 (s, 1H), 9.31 (s, 1H), 7.93 (d, J=8.1 Hz, 1H), 7.75 (dd, J=8.1, 4.3 Hz, 1H), 7.59 (d, J=7.6 Hz, 1H), 7.56-7.50 (m, 1H), 7.45 (t, J=7.7 Hz, 1H), 7.39-7.28 (m, 1H), 7.24 (d, J=7.8 Hz, 2H), 7.10 (d, J=8.2 Hz, 2H), 6.89 (s, 2H), 6.26-6.15 (m, 2H), 5.78 (d, J=10.8 Hz, 1H), 5.01-4.73 (m, 1H), 4.51-3.88 (m, 7H), 3.88-3.57 (m, 2H), 3.62 (s, 1H), 3.49 (s, 2H), 3.25-2.81 (m, 8H), 2.45-2.23 (m, 10H), 2.21-2.11 (m, 2H), 1.94 (s, 2H), 1.69-1.41 (m, 3H), 1.28 (s, 2H), 1.24 (s, 3H), 1.14 (s, 1H). LC-MS: m/z 979.2 [M+H]⁺.

### Compound 228

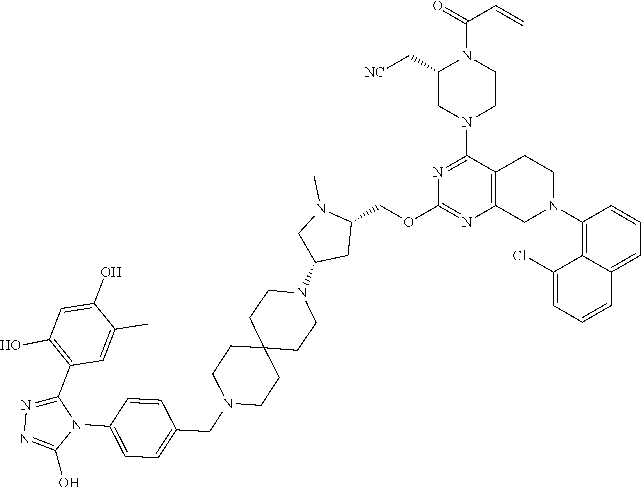 [SMILES: C=CC(=O)N1CCN(c2nc(OC[C@@H]3C[C@H](N4CCC5(CCN(Cc6ccc(-n7c(O)nnc7-c7cc(C)c(O)cc7O)cc6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-[9-({4-[3-(2,4-Dihydroxy-5-methylphenyl)-5-hydroxy-1,2,4-triazol-4-yl]phenyl}methyl)-3,9-diazaspiro[5.5]un decan-3-yl]-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.87 (s, 1H), 9.55 (s, 1H), 9.31 (s, 1H), 7.96-7.88 (m, 1H), 7.86-7.68 (m, 1H), 7.65-7.49 (m, 2H), 7.45 (t, J=7.8 Hz, 1H), 7.39-7.28 (m, 3H), 7.18-7.01 (m, 2H), 6.88 (d, J=0.9 Hz, 1H), 6.82-6.78 (m, 1H), 6.22 (d, J=8.1 Hz, 1H), 6.17 (s, 1H), 5.82-5.72 (m, 1H), 5.02-4.82 (d, 1H), 4.60-3.98 (m, 6H), 3.91-3.70 (m, 2H), 3.65-3.45 (m, 6H), 3.38 (s, 2H), 3.34-3.26 (m, 4H), 3.24-3.01 (m, 5H), 3.00-2.81 (m, 3H), 2.80-2.68 (m, 4H), 2.32-2.22 (m, 3H), 2.02-1.82 (m, 4H), 1.80-1.64 (m, 2H), 1.56 (s, 4H), 1.24 (s, 1H), 0.85 (d, J=7.0 Hz, 6H). LC-MS: m/z 1033.2 [M+H]⁺.

### Compound 229

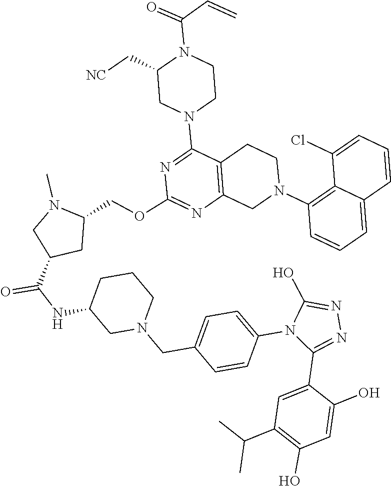 [SMILES: C=CC(=O)N1CCN(c2nc(OC[C@@H]3C[C@H](C(=O)N[C@@H]4CCCN(Cc5ccc(-n6c(O)nnc6-c6cc(C(C)C)c(O)cc6O)cc5)C4)CN3C)nc3c2CCN(c2cccc4cccc(Cl)c24)C3)C[C@@H]1CC#N]

### (3S,5S)-5-({[7-(8-chloronaphthalen-1-yl)-4-[(3S)-3-(cyanomethyl)-4-(prop-2-enoyl) piperazin-1-yl]-5H,6H,8H-pyrido[3,4-d]pyrimidin-2-yl]oxy}methyl)-N-[(3R)-1-({4-[3-(2,4-dihydroxy-5-isopropylphenyl)-5-hydroxy-1,2,4-triazol-4-yl]phenyl}methyl)piperidin-3-yl]-1-methylpyrrolidine-3-carboxamide

¹H NMR (DMSO-d₆, 400 MHz): δ 11.92 (s, 1H), 9.60 (s, 1H), 9.41 (s, 1H), 7.91 (dd, J=8.3, 1.3 Hz, 1H), 7.74 (dd, J=8.2, 3.8 Hz, 1H), 7.66 (d, J=7.8 Hz, 1H), 7.61-7.48 (m, 2H), 7.44 (t, J=7.8 Hz, 1H), 7.33 (dd, J=11.7, 7.5 Hz, 1H), 7.24 (d, J=8.1 Hz, 2H), 7.09 (d, J=8.0 Hz, 2H), 6.85 (s, 1H), 6.76 (s, 1H), 6.27 (s, 1H), 6.19 (dd, J=16.5, 2.3 Hz, 1H), 5.78 (dd, J=10.4, 2.3 Hz, 1H), 4.97-4.77 (m, 1H), 4.48-4.20 (m, 2H), 4.16-4.10 (m, 1H), 4.06-3.91 (m, 2H), 3.90-3.54 (m, 2H), 3.50-3.46 (m, 1H), 3.43-3.37 (m, 2H), 3.15-2.99 (m, 6H), 2.98-2.80 (m, 3H), 2.73 (t, J=8.5 Hz, 1H), 2.69-2.54 (m, 2H), 2.39 (t, J=8.8 Hz, 3H), 2.35-2.26 (m, 3H), 2.19-2.13 (m, 1H), 2.00 (s, 2H), 1.77 (s, 1H), 1.62-1.54 (d, J=15.3 Hz, 2H), 1.41 (d, J=10.6 Hz, 1H), 1.31-1.10 (m, 1H), 0.92 (dd, J=6.9, 2.0 Hz, 6H). LC-MS: m/z 1035.2 [M+H]⁺.

### Compound 230

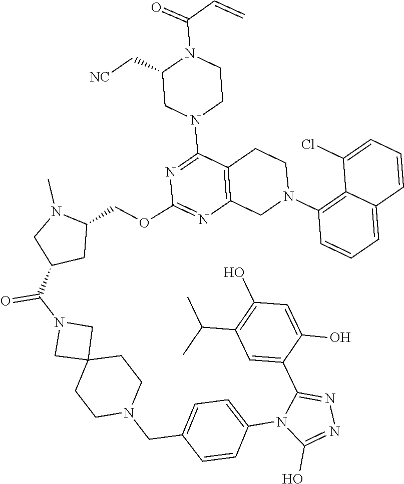 [SMILES: C=CC(=O)N1CCN(c2nc(OC[C@@H]3C[C@H](C(=O)N4CC5(CCN(Cc6ccc(-n7c(O)nnc7-c7cc(C(C)C)c(O)cc7O)cc6)CC5)C4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-[7-({4-[3-(2,4-dihydroxy-5-isopropylphenyl)-5-hydroxy-1,2,4-triazol-4-yl]phenyl}methyl)-2,7-diazaspiro[3.5]nonane-2-carbonyl]-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.93 (s, 1H), 9.60 (s, 1H), 9.42 (s, 1H), 7.93 (d, J=8.1 Hz, 1H), 7.75 (dd, J=8.1, 4.4 Hz, 1H), 7.62-7.48 (m, 2H), 7.45 (t, J=7.8 Hz, 1H), 7.35 (dd, J=18.0, 7.4 Hz, 1H), 7.28 (d, J=8.2 Hz, 2H), 7.16-7.08 (m, 2H), 6.83 (d, J=27.4 Hz, 1H), 6.76 (s, 1H), 6.27 (s, 1H), 6.19 (d, J=16.6 Hz, 1H), 5.78 (d, J=11.8 Hz, 1H), 4.97-4.77 (m, 1H), 4.48-3.91 (m, 6H), 3.90-3.57 (m, 4H), 3.50 (s, 3H), 3.39 (s, 2H), 3.12-3.06 (m, 2H), 2.97 (q, J=6.9 Hz, 3H), 2.93-2.82 (m, 2H), 2.68 (p, J=1.9 Hz, 1H), 2.58 (d, J=7.2 Hz, 2H), 2.40 (t, J=9.0 Hz, 1H), 2.35-2.19 (m, 6H), 2.09 (q, J=9.8, 8.2 Hz, 1H), 1.84-1.73 (m, 1H), 1.66 (s, 4H), 0.94 (d, J=6.9 Hz, 6H). LC-MS: m/z 1061.2 [M+H]⁺.

### Compound 231

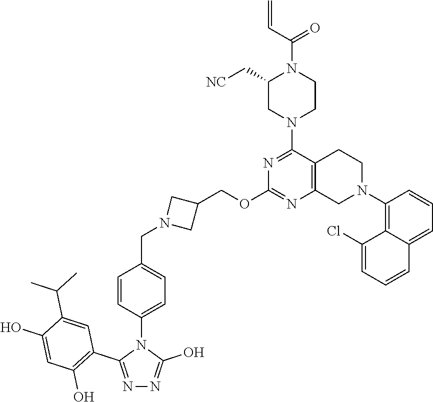 [SMILES: C=CC(=O)N1CCN(c2nc(OCC3CN(Cc4ccc(-n5c(O)nnc5-c5cc(C(C)C)c(O)cc5O)cc4)C3)nc3c2CCN(c2cccc4cccc(Cl)c24)C3)C[C@@H]1CC#N]

### (S)-2-(1-acryloyl-4-(7-(8-chloronaphthalen-1-yl)-2-((1-(4-(3-(2,4-dihydroxy-5-isopropylphenyl)-5-hydroxy-4H-1,2,4-triazol-4-yl)benzyl)azetidin-3-yl)methoxy)-5,6,7,8-tetrahydropyrido[3,4-d]pyrimidin-4-yl)piperazin-2-yl)acetonitrile

¹H NMR (400 MHz, MeOD) δ 7.83 (d, J=8.0 Hz, 1H), 7.70 (d, J=8.0 Hz, 1H), 7.56-7.47 (m, 4H), 7.37 (dd, J=15.1, 7.7 Hz, 4H), 6.86-6.76 (m, 2H), 6.29 (d, J=16.8 Hz, 1H), 6.23-6.18 (m, 1H), 5.83 (d, J=10.7 Hz, 1H), 4.54-4.43 (m, 5H), 4.39-4.31 (m, 2H), 4.28-4.11 (m, 6H), 3.74 (dd, J=16.3, 7.5 Hz, 1H), 3.66-3.53 (m, 2H), 3.25-3.16 (m, 2H), 3.13-2.98 (m, 3H), 2.94-2.87 (m, 1H), 2.83-2.70 (m, 1H), 1.32-1.26 (m, 2H), 0.97 (d, J=6.9 Hz, 6H). LC-MS: m/z 881.2 [M+H]⁺.

### Compound 232

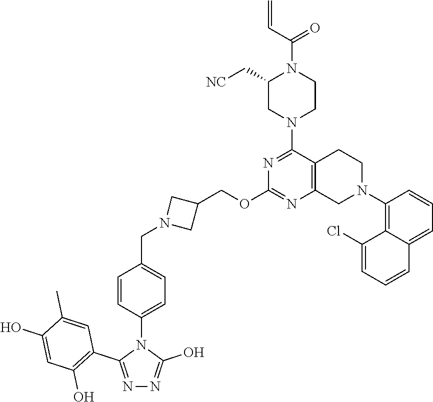 [SMILES: C=CC(=O)N1CCN(c2nc(OCC3CN(Cc4ccc(-n5c(O)nnc5-c5cc(C)c(O)cc5O)cc4)C3)nc3c2CCN(c2cccc4cccc(Cl)c24)C3)C[C@@H]1CC#N]

### (S)-2-(1-acryloyl-4-(7-(8-chloronaphthalen-1-yl)-2-((1-(4-(3-(2,4-dihydroxy-5-methylphenyl)-5-hydroxy-4H-1,2,4-triazol-4-yl)benzyl)azetidin-3-yl)methoxy)-5,6,7,8-tetrahydropyrido[3,4-d]pyrimidin-4-yl)piperazin-2-yl)acetonitrile

¹H NMR (400 MHz, MeOD) δ 7.84 (d, J=8.0 Hz, 1H), 7.70 (d, J=8.1 Hz, 1H), 7.56-7.45 (m, 4H), 7.42-7.28 (m, 4H), 6.90-6.78 (m, 2H), 6.29 (d, J=16.3 Hz, 1H), 6.20-6.13 (m, 1H), 5.84 (d, J=10.4 Hz, 1H), 4.58-4.40 (m, 5H), 4.39-4.08 (m, 8H), 3.81-3.70 (m, 1H), 3.68-3.50 (m, 2H), 3.27-3.18 (m, 2H), 3.14-2.87 (m, 3H), 2.84-2.68 (m, 1H), 1.98 (s, 3H), 1.32-1.26 (m, 2H). LC-MS: m/z 853.3 [M+H]⁺.

### Compound 233

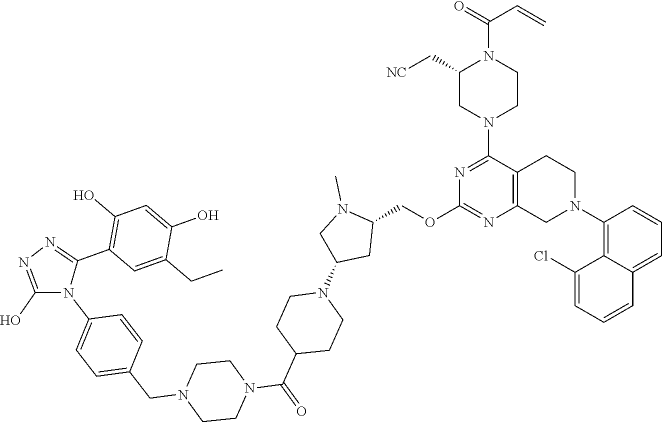 [SMILES: C=CC(=O)N1CCN(c2nc(OC[C@@H]3C[C@H](N4CCC(C(=O)N5CCN(Cc6ccc(-n7c(O)nnc7-c7cc(CC)c(O)cc7O)cc6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{4-[4-({4-[3-(5-ethyl-2,4-dihydroxyphenyl)-5-hydroxy-1,2,4-triazol-4-yl]phenyl}methyl)piperazine-1-carbonyl]piperidin-1-yl}-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.90 (s, 1H), 9.56 (s, 1H), 9.35 (s, 1H), 7.92 (d, J=8.2 Hz, 1H), 7.74 (dd, J=8.1, 4.3 Hz, 1H), 7.60-7.50 (m, 2H), 7.45 (t, J=7.8 Hz, 1H), 7.34 (dd, J=17.6, 7.5 Hz, 1H), 7.28 (d, J=8.2 Hz, 2H), 7.14-7.09 (m, 2H), 6.83 (s, 2H), 6.24 (s, 1H), 6.21-6.16 (m, 1H), 5.88-5.72 (m, 1H), 4.96-4.76 (m, 1H), 4.40-4.12 (m, 4H), 4.03-3.94 (m, 3H), 3.83-3.49 (m, 3H), 3.44 (s, 7H), 3.25-3.01 (m, 4H), 3.00-2.93 (m, 2H), 2.91-2.71 (m, 4H), 2.41-2.23 (m, 10H), 2.12-1.91 (m, 3H), 1.53 (s, 5H), 1.24 (s, 1H), 0.97 (t, J=7.5 Hz, 3H). LC-MS: m/z 1090.2 [M+H]⁺.

### Compound 234

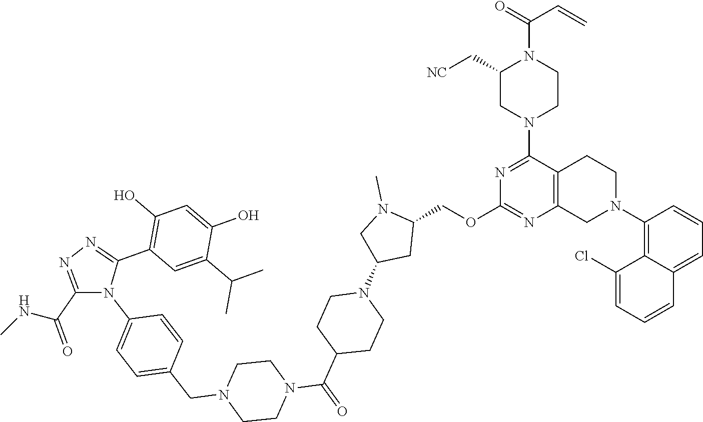 [SMILES: C=CC(=O)N1CCN(c2nc(OC[C@@H]3C[C@H](N4CCC(C(=O)N5CCN(Cc6ccc(-n7c(C(=O)NC)nnc7-c7cc(C(C)C)c(O)cc7O)cc6)CC5)CC4)CN3C)nc3c2CCN(c2cccc4cccc(Cl)c24)C3)C[C@@H]1CC#N]

### 4-{4-[(4-{1-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}meth yl)-1-methylpyrrolidin-3-yl]piperidine-4-carbonyl}piperazin-1-yl)methyl]phenyl}-5-(2,4-dihydroxy-5-isopropylphenyl)-N-methyl-1,2,4-triazole-3-carboxamide

¹H NMR (DMSO-d₆, 400 MHz): δ 10.55 (s, 1H), 9.76 (s, 1H), 8.89 (d, J=4.8 Hz, 1H), 7.92 (d, J=8.1 Hz, 1H), 7.74 (dd, J=8.1, 4.3 Hz, 1H), 7.61-7.50 (m, 1H), 7.45 (t, J=7.8 Hz, 1H), 7.44-7.36 (m, 1H), 7.33-7.27 (m, 5H), 6.85 (s, 1H), 6.59 (s, 1H), 6.34 (s, 1H), 6.20-6.16 (m, 1H), 5.79-5.76 (m, 1H), 4.79-4.76 (m, 1H), 4.51-4.11 (m, 4H), 4.08-3.95 (m, 2H), 3.91-3.66 (m, 2H), 3.58-3.41 (m, 8H), 3.24-2.74 (m, 12H), 2.68 (t, J=3.2 Hz, 4H), 2.38 (s, 2H), 2.35-2.21 (m, 6H), 2.08-1.93 (m, 3H), 1.54 (s, 5H), 0.81 (d, J=6.8 Hz, 6H). LC-MS: m/z 1145.2 [M+H]⁺.

### Compound 235

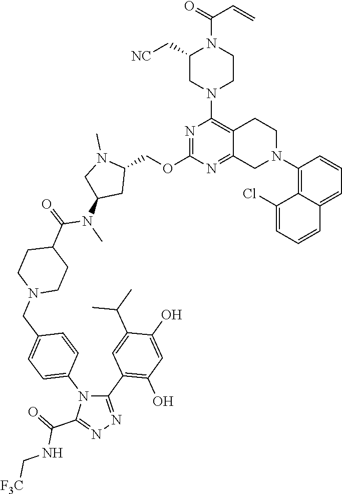 [SMILES: C=CC(=O)N1CCN(c2nc(OC[C@@H]3C[C@@H](N(C)C(=O)C4CCN(Cc5ccc(-n6c(C(=O)NCC(F)(F)F)nnc6-c6cc(C(C)C)c(O)cc6O)cc5)CC4)CN3C)nc3c2CCN(c2cccc4cccc(Cl)c24)C3)C[C@@H]1CC#N]

### N-((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)-1-(4-(3-(2,4-dihydroxy-5-isopropylphenyl)-5-((2,2,2-trifluoroethyl)carbamoyl)-4H-1,2,4-triazol-4-yl)benzyl)-N-methylpiperidine-4-carboxamide

¹H NMR (400 MHz, MeOD) δ 7.85 (d, J=8.1 Hz, 1H), 7.73 (d, J=8.0 Hz, 1H), 7.64 (d, J=8.3 Hz, 2H), 7.58-7.47 (m, 5H), 7.46-7.30 (m, 3H), 6.76 (s, 1H), 6.33-6.28 (m, 2H), 5.84 (d, J=10.4 Hz, 1H), 4.79-4.70 (m, 2H), 4.62-4.23 (m, 8H), 4.03-3.94 (m, 3H), 3.90-3.78 (m, 3H), 3.66-3.52 (m, 5H), 3.20-2.89 (m, 15H), 2.11-1.90 L10 (m, 5H), 1.34-1.27 (m, 1H), 0.93 (d, J=6.9 Hz, 6H). LC-MS: m/z 1158.6 [M+H]⁺.

### Compound 236

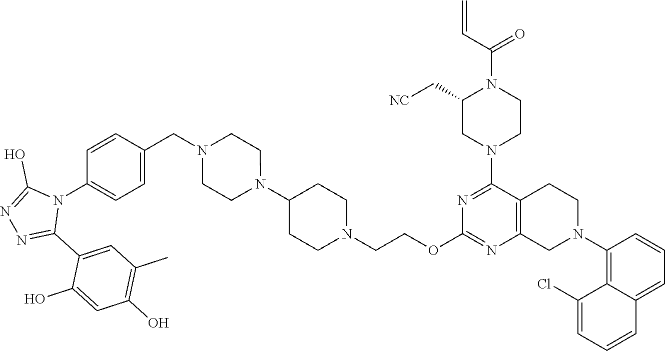 [SMILES: C=CC(=O)N1CCN(c2nc(OCCN3CCC(N4CCN(Cc5ccc(-n6c(O)nnc6-c6cc(C)c(O)cc6O)cc5)CC4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(4-(4-(3-(2,4-dihydroxy-5-methylphenyl)-5-hydroxy-4H-1,2,4-triazol-4-yl)benzyl)piperazin-1-yl)piperidin-1-yl)ethoxy)-5,6,7,8-tetrahydropyrido[3,4-d]pyrimidin-4-yl)piperazin-2-yl)acetonitrile

¹H NMR (400 MHz, MeOD) δ 7.85 (d, J=8.0 Hz, 1H), 7.73 (d, J=8.0 Hz, 1H), 7.53 (dd, J=15.5, 7.7 Hz, 4H), 7.42-7.33 (m, 4H), 6.91-6.74 (m, 2H), 6.31 (d, J=16.8 Hz, 1H), 6.19 (s, 1H), 5.85 (d, J=10.4 Hz, 1H), 4.56-4.14 (m, 6H), 3.87-3.39 (m, 11H), 3.23 (s, 7H), 3.12-2.67 (m, 9H), 2.18-2.11 (m, 2H), 2.03-1.94 (m, 5H), 1.31 (m, 1H). LC-MS: m/z 979.4 [M+H]⁺.

### Compound 237

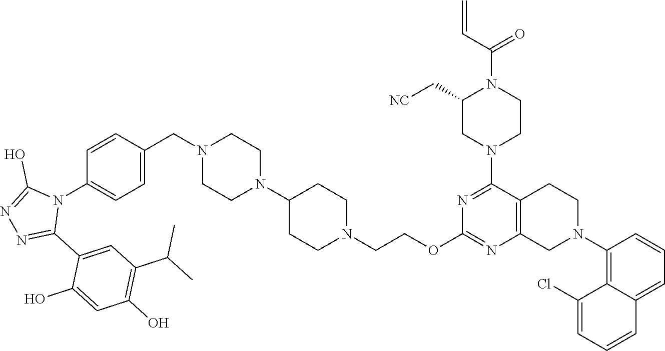 [SMILES: C=CC(=O)N1CCN(c2nc(OCCN3CCC(N4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(4-(4-(3-(2,4-dihydroxy-5-isopropylphenyl)-5-hydroxy-4H-1,2,4-triazol-4-yl)benzyl)piperazin-1-yl)piperidin-1-yl)ethoxy)-5,6,7,8-tetrahydropyrido[3,4-d]pyrimidin-4-yl)piperazin-2-yl)acetonitrile

¹H NMR (400 MHz, MeOD) δ 7.84 (d, J=8.0 Hz, 1H), 7.71 (d, J=8.2 Hz, 1H), 7.52 (ddd, J=12.8, 10.1, 6.7 Hz, 4H), 7.44-7.28 (m, 4H), 6.84 (s, 2H), 6.34-6.19 (m, 2H), 5.84 (d, J=10.1 Hz, 1H), 4.81 (s, 3H), 4.51-4.00 (m, 6H), 3.77 (m, 4H), 3.61 (s, 4H), 3.19 (m, 7H), 3.06 (m, 2H), 3.00-2.73 (m, 6H), 2.21-1.86 (m, 5H), 1.33 (m, 5H), 0.99 (d, J=6.9 Hz, 6H). LC-MS: m/z 1007.5 [M+H]⁺.

### Compound 238

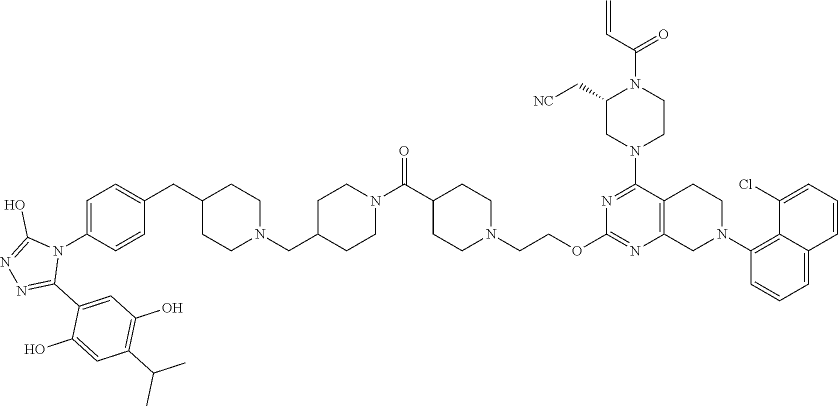 [SMILES: C=CC(=O)N1CCN(c2nc(OCCN3CCC(C(=O)N4CCC(CN5CCC(Cc6ccc(-n7c(O)nnc7-c7cc(O)c(C(C)C)cc7O)cc6)CC5)CC4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(4-((4-(4-(3-(2,5-dihydroxy-4-isopropylphenyl)-5-hydroxy-4H-1,2,4-triazol-4-yl)benzyl)piperidin-1-yl)methyl)piperidine-1-carbonyl)piperidin-1-yl)ethoxy)-5,6,7,8-tetrahydropyrido[3,4-d]pyrimidin-4-yl)piperazin-2-yl)acetonitrile

¹H NMR (400 MHz, MeOD) δ 7.97 (t, J=7.0 Hz, 1H), 7.82 (d, J=2.9 Hz, 1H), 7.67 (s, 1H), 7.53-7.48 (m, 2H), 7.36 (d, J=8.2 Hz, 1H), 7.30 (d, J=8.4 Hz, 2H), 7.21 (d, J=8.3 Hz, 2H), 6.74 (d, J=47.0 Hz, 2H), 6.29 (d, J=10.8 Hz, 2H), 5.84 (s, 1H), 4.55 (d, J=23.4 Hz, 6H), 4.30 (d, J=16.8 Hz, 2H), 4.17 (s, 1H), 4.05 (s, 2H), 3.77-3.58 (m, 3H), 3.48 (s, 1H), 3.13 (s, 8H), 2.99 (d, J=6.7 Hz, 1H), 2.90 (s, 3H), 2.71 (s, 2H), 2.62 (d, J=6.6 Hz, 3H), 2.36 (s, 2H), 2.18 (d, J=7.8 Hz, 1H), 2.03 (s, 2H), 1.76 (d, J=15.0 Hz, 5H), 1.29 (s, 6H), 1.17 (s, 3H), 0.88 (d, J=6.9 Hz, 6H) LC-MS: m/z 1131.6 [M+H]⁺.

### Compound 239

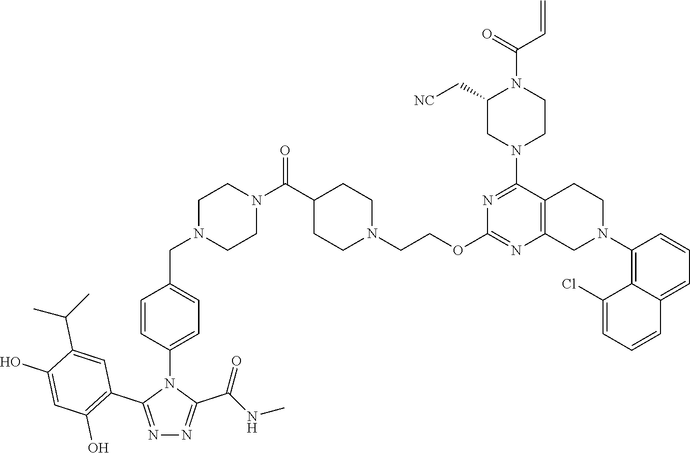 [SMILES: C=CC(=O)N1CCN(c2nc(OCCN3CCC(C(=O)N4CCN(Cc5ccc(-n6c(C(=O)NC)nnc6-c6cc(C(C)C)c(O)cc6O)cc5)CC4)CC3)nc3c2CCN(c2cccc4cccc(Cl)c24)C3)C[C@@H]1CC#N]

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-isopropylphenyl)-N-methyl-4H-1,2,4-triazole-3-carboxamide

¹H NMR (400 MHz, MeOD) δ 7.84 (d, J=8.0 Hz, 1H), 7.71 (d, J=8.4 Hz, 1H), 7.62 (d, J=8.3 Hz, 2H), 7.55-7.47 (m, 4H), 7.41-7.31 (m, 2H), 6.89-6.72 (m, 2H), 6.30 (d, J=16.4 Hz, 2H), 5.84 (d, J=10.3 Hz, 1H), 4.81-4.73 (m, 3H), 4.44-4.30 (m, 4H), 4.27-4.06 (m, 2H), 4.02-3.67 (m, 7H), 3.65-3.54 (m, 4H), 3.42-3.33 (m, 4H), 3.27-2.98 (m, 8H), 2.96-2.89 (m, 1H), 2.84 (s, 3H), 2.81-2.70 (m, 1H), 2.08-2.00 (m, 4H), 1.34-1.28 (m, 1H), 0.93 (d, J=6.9 Hz, 6H). LC-MS: m/z 1076.5 [M+H]⁺.

### Compound 240

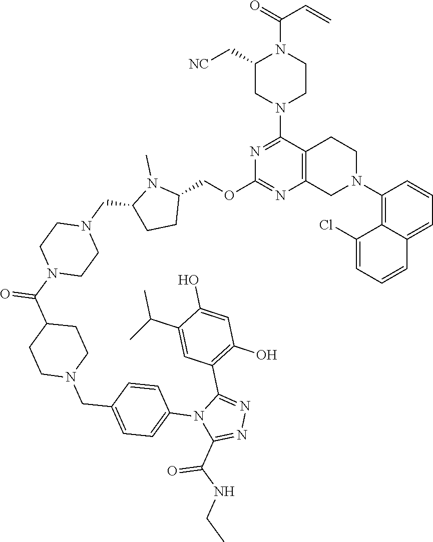 [SMILES: C=CC(=O)N1CCN(c2nc(OC[C@@H]3CC[C@H](CN4CCN(C(=O)C5CCN(Cc6ccc(-n7c(C(=O)NCC)nnc7-c7cc(C(C)C)c(O)cc7O)cc6)CC5)CC4)N3C)nc3c2CCN(c2cccc4cccc(Cl)c24)C3)C[C@@H]1CC#N]

### 4-(4-{[4-(4-{[(2R,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}meth yl)-1-methylpyrrolidin-2-yl]methyl}piperazine-1-carbonyl)piperidin-1-yl]methyl}phenyl)-5-(2,4-dihydroxy-5-isopropylphenyl)-N-ethyl-1,2,4-triazole-3-carboxamide

¹H NMR (DMSO-d₆, 400 MHz): δ 10.62 (s, 1H), 9.75 (s, 1H), 8.96 (t, J=5.9 Hz, 1H), 7.92 (d, J=8.1 Hz, 1H), 7.74 (dd, J=8.0, 4.4 Hz, 1H), 7.61-7.50 (m, 2H), 7.45 (t, J=7.8 Hz, 1H), 7.40-7.34 (m, 3H), 7.29 (d, J=8.4 Hz, 2H), 6.85 (s, 1H), 6.58 (s, 1H), 6.34 (s, 1H), 6.18 (dd, J=16.7, 2.3 Hz, 1H), 5.77 (dd, J=10.4, 2.3 Hz, 1H), 5.03-4.72 (m, 1H), 4.52-4.09 (m, 3H), 4.09-3.92 (m, 3H), 3.88-3.61 (m, 2H), 3.46 (d, J=8.2 Hz, 7H), 3.27-2.98 (m, 7H), 2.97-2.85 (m, 2H), 2.84-2.79 (m, 2H), 2.78-2.61 (m, 2H), 2.60-2.52 (m, 2H), 2.47-2.25 (m, 8H), 2.20 (dd, J=12.6, 6.8 Hz, 1H), 1.99 (s, 2H), 1.85 (s, 2H), 1.73-1.32 (m, 6H), 1.03 (t, J=7.2 Hz, 3H), 0.80 (d, J=6.8 Hz, 6H). LC-MS: m/z 1173.1 [M+H]⁺.

### Compound 241

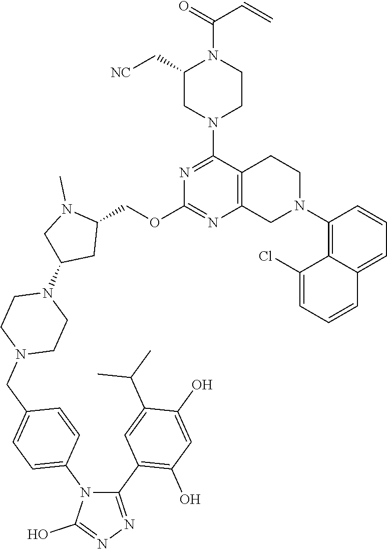 [SMILES: C=CC(=O)N1CCN(c2nc(OC[C@@H]3C[C@H](N4CCN(Cc5ccc(-n6c(O)nnc6-c6cc(C(C)C)c(O)cc6O)cc5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-[4-({4-[3-(2,4-dihydroxy-5-isopropylphenyl)-5-hydroxy-1,2,4-triazol-4-yl]phenyl}methyl)piperazin-1-yl]-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.95 (s, 1H), 9.60 (s, 1H), 9.39 (s, 1H), 7.92 (d, J=8.0 Hz, 1H), 7.82-7.70 (m, 1H), 7.60-7.51 (m, 2H), 7.48-7.41 (m, 1H), 7.40-7.34 (m, 1H), 7.29-7.24 (m, 2H), 7.23 (s, 2H), 7.15-7.08 (m, 1H), 6.97 (s, 2H), 6.27 (s, 1H), 6.19 (d, J=16.5 Hz, 1H), 5.78 (d, J=10.6 Hz, 1H), 4.96-4.70 (m, 1H), 4.60-4.14 (m, 4H), 4.12-3.95 (m, 2H), 3.91-3.65 (m, 2H), 3.50-3.46 (m, 3H), 3.25-2.82 (m, 12H), 2.80-2.75 (m, 1H), 2.52-2.10 (m, 9H), 1.88-1.81 (m, 1H), 1.11-0.80 (m, 6H). LC-MS: m/z 993.2 [M+H]⁺.

### Compound 242

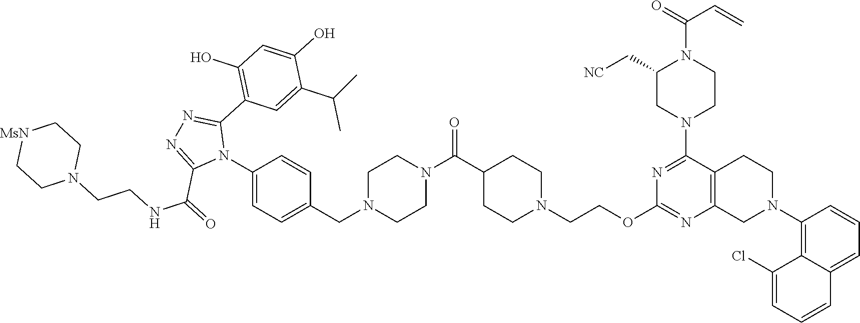

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-isopropylphenyl)-N-(2-(4-(methylsulfonyl)piperazin-1-yl)ethyl)-4H-1,2,4-triazole-3-carboxamide

¹H NMR (400 MHz, MeOD) δ 7.82 (d, J=7.6 Hz, 1H), 7.68 (d, J=8.1 Hz, 1H), 7.50 (dd, J=16.1, 7.9 Hz, 4H), 7.38-7.29 (m, 4H), 6.74 (d, J=56.5 Hz, 2H), 6.28 (d, J=18.4 Hz, 2H), 5.83 (d, J=10.4 Hz, 1H), 4.58 (s, 4H), 4.50 (s, 2H), 4.30 (d, J=17.0 Hz, 2H), 4.16 (s, 1H), 4.10-4.02 (m, 1H), 3.71 (s, 1H), 3.61 (s, 7H), 3.43 (m, 3H), 3.20 (s, 5H), 3.09 (s, 3H), 3.04-2.95 (m, 2H), 2.91 (m, 1H), 2.84 (s, 4H), 2.67 (s, 2H), 2.57 (m, 5H), 2.49 (m, 4H), 2.28 (s, 2H), 1.81-1.70 (m, 4H), 1.29 (s, 1H), 0.87 (d, J=6.9 Hz, 6H). LC-MS: m/z 1253.0 [M+H]⁺.

### Compound 243

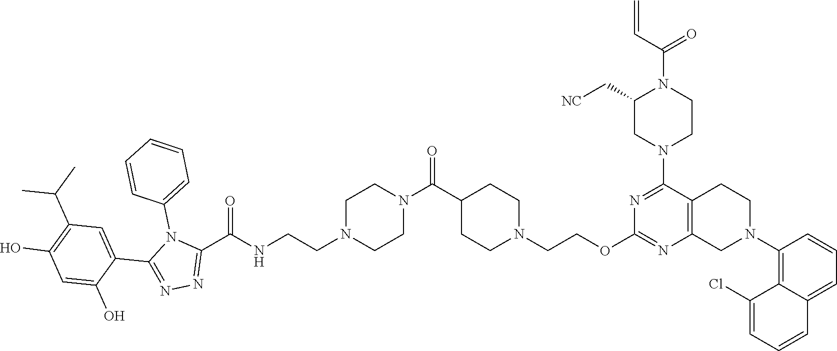 [SMILES: C=CC(=O)N1CCN(c2nc(OCCN3CCC(C(=O)N4CCN(CCNC(=O)c5nnc(-c6cc(C(C)C)c(O)cc6O)n5-c5ccccc5)CC4)CC3)nc3c2CCN(c2cccc4cccc(Cl)c24)C3)C[C@@H]1CC#N]

### (S)—N-(2-(4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)ethyl)-5-(2,4-dihydroxy-5-isopropylphenyl)-4-phenyl-4H-1,2,4-triazole-3-carboxamide

¹H NMR (400 MHz, MeOD) δ 7.81 (d, J=8.1 Hz, 1H), 7.67 (d, J=7.7 Hz, 1H), 7.53-7.45 (m, 5H), 7.35 (ddd, J=15.9, 10.2, 4.3 Hz, 4H), 6.71 (d, J=72.7 Hz, 2H), 6.29 (d, J=20.7 Hz, 2H), 5.82 (d, J=10.6 Hz, 1H), 4.58 (s, 1H), 4.49 (t, J=5.5 Hz, 2H), 4.33-4.27 (m, 1H), 4.17 (m, 2H), 3.72 (m, 1H), 3.55 (s, 5H), 3.43 (m, 3H), 3.25-2.87 (m, 9H), 2.80 (m, 2H), 2.67 (m, 2H), 2.58-2.43 (m, 6H), 2.24 (t, J=11.6 Hz, 2H), 1.73 (m, 4H), 1.31 (m, 1H), 0.85 (d, J=6.9 Hz, 6H). LC-MS: m/z 1076.5 [M+H]⁺.

### Compound 244

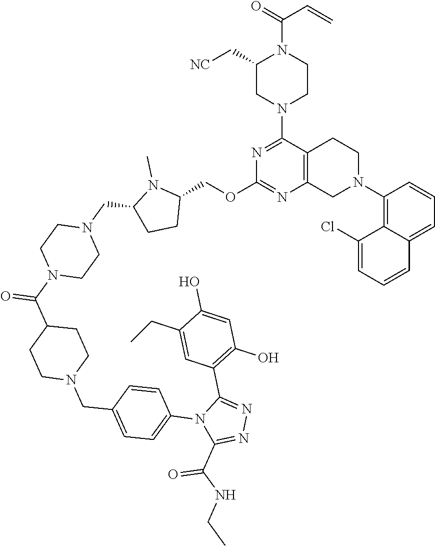 [SMILES: C=CC(=O)N1CCN(c2nc(OC[C@@H]3CC[C@H](CN4CCN(C(=O)C5CCN(Cc6ccc(-n7c(C(=O)NCC)nnc7-c7cc(CC)c(O)cc7O)cc6)CC5)CC4)N3C)nc3c2CCN(c2cccc4cccc(Cl)c24)C3)C[C@@H]1CC#N]

### 4-(4-{[4-(4-{[(2R,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-2-yl]methyl}piperazine-1-carbonyl)piperidin-1-yl]methyl}phenyl)-N-ethyl-5-(5-ethyl-2,4-dihydroxyphenyl)-1,2,4-triazole-3-carboxamide

¹H NMR (DMSO-d₆, 400 MHz): δ 10.39 (s, 1H), 9.68 (s, 1H), 8.94 (t, J=5.9 Hz, 1H), 7.92 (d, J=8.1 Hz, 1H), 7.74 (dd, J=8.1, 4.4 Hz, 1H), 7.61-7.48 (m, 2H), 7.45 (t, J=7.8 Hz, 1H), 7.37-7.28 (m, 3H), 7.25 (d, J=8.0 Hz, 2H), 6.84 (s, 1H), 6.53 (s, 1H), 6.32 (s, 1H), 6.18 (d, J=16.8 Hz, 1H), 5.77 (d, J=10.6 Hz, 1H), 4.99-4.75 (m, 1H), 4.46-4.11 (m, 3H), 4.01 (s, 3H), 3.89-3.68 (m, 3H), 3.47 (s, 8H), 3.17 (p, J=7.2 Hz, 2H), 3.12-2.95 (m, 8H), 2.92-2.85 (m, 1H), 2.80 (d, J=10.7 Hz, 2H), 2.72-2.68 (m, 1H), 2.37 (s, 5H), 2.29 (s, 1H), 2.21 (q, J=7.5 Hz, 3H), 1.99 (s, 2H), 1.85 (s, 2H), 1.56 (s, 6H), 1.04 (t, J=7.2 Hz, 3H), 0.83 (t, J=7.5 Hz, 3H). LC-MS: m/z 1159.2 [M+H]⁺.

### Compound 245

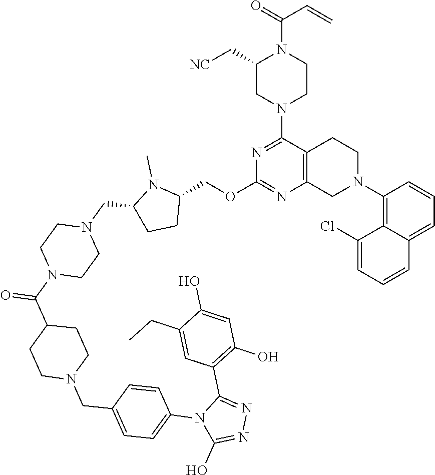 [SMILES: C=CC(=O)N1CCN(c2nc(OC[C@@H]3CC[C@H](CN4CCN(C(=O)C5CCN(Cc6ccc(-n7c(O)nnc7-c7cc(CC)c(O)cc7O)cc6)CC5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-({4-[1-({4-[3-(5-ethyl-2,4-di hydroxyphenyl)-5-hydroxy-1,2,4-triazol-4-yl]phenyl}methyl)piperidine-4-carbon yl]piperazin-1-yl}methyl)-1-methylpyrrolidin-2-yl]methoxy}-5H,6H,8H-pyrido [3,4-d]pyrimidin-4-yl]-1-(prop-2-enoyl)piperazin-2-yl]acetonitrile

¹H NMR (DMSO-d₆,400 MHz): δ 11.88 (s, 1H), 9.53 (s, 1H), 9.35 (s, 1H), 7.92 (d, J=8.1 Hz, 1H), 7.74 (t, J=6.4 Hz, 1H), 7.54 (dt, J=16.3, 7.9 Hz, 2H), 7.45 (t, J=7.7 Hz, 1H), 7.41-7.28 (m, 1H), 7.26 (d, J=8.0 Hz, 2H), 7.11 (d, J=8.0 Hz, 2H), 6.80 (s, 2H), 6.24 (s, 1H), 6.18 (d, J=16.6 Hz, 1H), 5.77 (d, J=10.8 Hz, 1H), 5.01-4.72 (m, 1H), 4.45-4.13 (m, 2H), 4.10-3.60 (m, 5H), 3.41 (s, 6H), 3.24-2.84 (m, 6H), 2.76 (d, J=10.5 Hz, 2H), 2.72-2.61 (m, 2H), 2.43-2.27 (m, 12H), 2.24-2.11 (m, 2H), 1.95 (s, 2H), 1.84 (s, 2H), 1.53 (s, 6H), 1.24 (s, 1H), 0.95 (t, J=7.4 Hz, 3H). LC-MS: m/z 1104.7 [M+H]⁺.

### Compound 246

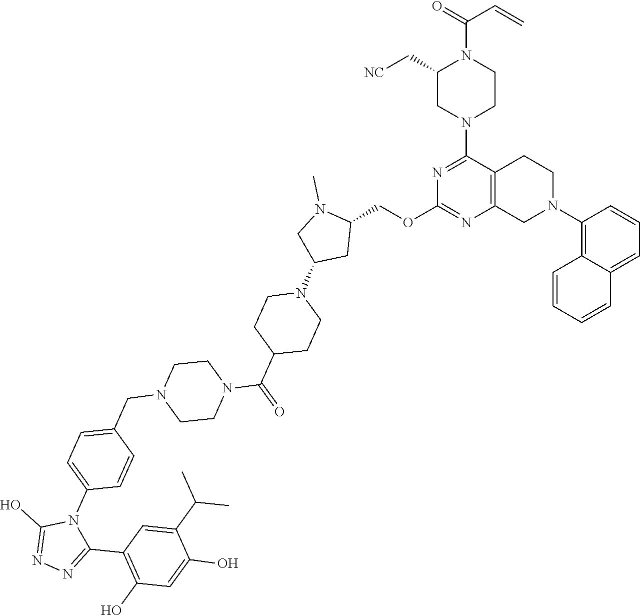

### 2-[(2S)-4-(2-{[(2S,4S)-4-{4-[4-({4-[3-(2,4-dihydroxy-5-isopropylphenyl)-5-hydro-xy-1,2,4-triazol-4-yl]phenyl}methyl)piperazine-1-carbonyl]piperidin-1-yl}-1-methylpyrrolidin-2-yl]methoxy}-7-(naphthalen-1-yl)-5H,6H,8H-pyrido[3,4-d]pyrimidin-4-yl)-1-(prop-2-enoyl)piperazin-2-yl]acetonitrile. ¹H NMR (DMSO-d₆, 400 MHz): δ 11.91 (s, 1H), 9.57 (s, 1H), 9.38 (s, 1H), 8.22-8.15 (m, 1H), 7.96-7.89 (m, 1H), 7.64 (d, J=8.2 Hz, 1H), 7.58-7.50 (m, 2H), 7.46 (t, J=7.8 Hz, 1H), 7.30 (d, J=8.3 Hz, 2H), 7.23 (d, J=7.4 Hz, 1H), 7.17-7.10 (m, 2H), 6.86 (s, 1H), 6.77 (s, 1H), 6.26 (s, 1H), 6.23-6.14 (m, 1H), 5.83-5.74 (m, 1H), 5.08-4.72 (m, 1H), 4.35-4.27 (m, 3H), 4.24-4.11 (m, 2H), 4.10-3.94 (m, 3H), 3.45 (s, 7H), 3.27-3.10 (m, 3H), 3.09-2.91 (m, 7H), 2.88-2.79 (m, 2H), 2.67 (p, J=1.8 Hz, 2H), 2.52 (d, J=1.8 Hz, 8H), 2.19-1.85 (m, 3H), 1.54 (s, 5H), 0.94 (d, J=6.9 Hz, 6H). LC-MS: m/z 1070.4 [M+H]⁺

### Compound 247

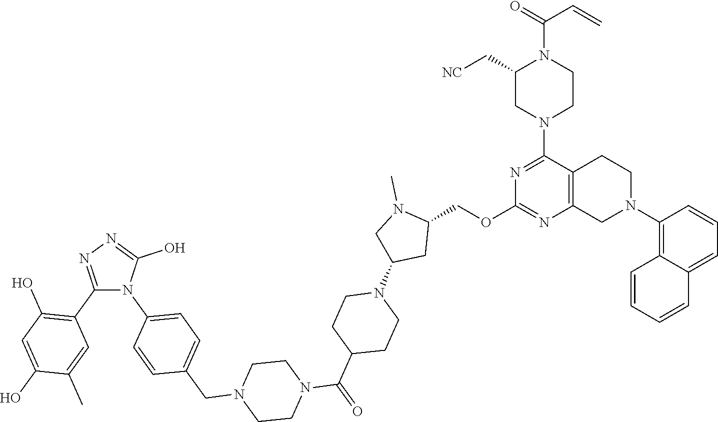

### 2-[(2S)-4-(2-{[(2S,4S)-4-{4-[4-({4-[3-(2,4-dihydroxy-5-methylphenyl)-5-hydroxy-1,2,4-triazol-4-yl]phenyl}methyl)piperazine-1-carbonyl]piperidin-1-yl}-1-methyl pyrrolidin-2-yl]methoxy}-7-(naphthalen-1-yl)-5H,6H,8H-pyrido[3,4-d]pyrimidin-4-yl)-1-(prop-2-enoyl)piperazin-2-yl]acetonitrile

¹H NMR (DMSO-d₆, 400 MHz): δ 11.86 (s, 1H), 9.54 (s, 1H), 9.30 (s, 1H), 8.22-8.15 (m, 1H), 7.92 (dt, J=7.8, 2.7 Hz, 1H), 7.64 (d, J=8.2 Hz, 1H), 7.59-7.50 (m, 2H), 7.46 (t, J=7.8 Hz, 1H), 7.30-7.20 (m, 3H), 7.14-7.08 (m, 2H), 6.89 (s, 2H), 6.22 (d, J=5.7 Hz, 1H), 6.17 (d, J=2.3 Hz, 1H), 5.79-5.76 (m, 1H), 4.98-4.77 (m, 1H), 4.29 (d, J=4.9 Hz, 2H), 4.22-3.93 (m, 5H), 3.44 (s, 8H), 3.31 (s, 3H), 3.11-2.79 (m, 8H), 2.69-2.54 (m, 2H), 2.41-2.21 (m, 8H), 1.94 (s, 6H), 1.54 (s, 5H). LC-MS: m/z 1042.4 [M+H]⁺.

### Compound 248

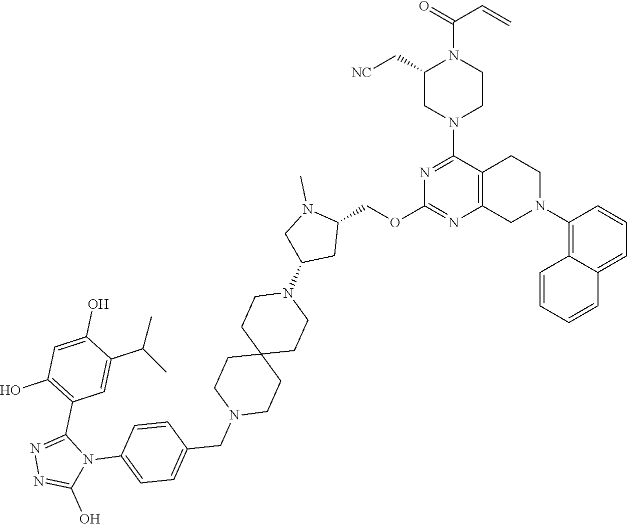 [SMILES: C=CC(=O)N1CCN(c2nc(OC[C@@H]3C[C@H](N4CCC5(CCN(Cc6ccc(-n7c(O)nnc7-c7cc(C(C)C)c(O)cc7O)cc6)CC5)CC4)CN3C)nc3c2CCN(c2cccc4ccccc24)C3)C[C@@H]1CC#N]

### 2-[(2S)-4-(2-{[(2S,4S)-4-[9-({4-[3-(2,4-dihydroxy-5-isopropylphenyl)-5-hydroxy-1,2,4-triazol-4-yl]phenyl}methyl)-3,9-diazaspiro[5.5]undecan-3-yl]-1-methylpyrr olidin-2-yl]methoxy}-7-(naphthalen-1-yl)-5H,6H,8H-pyrido[3,4-d]pyrimidin-4-yl)-1-(prop-2-enoyl)piperazin-2-yl]acetonitrile

¹H NMR (DMSO-d₆, 400 MHz): δ 11.91 (s, 1H), 9.57 (s, 1H), 9.40 (s, 1H), 8.19-8.17 (m, 1H), 7.92 (dt, J=7.8, 2.7 Hz, 1H), 7.64 (d, J=8.2 Hz, 1H), 7.55-7.52 (m, 2H), 7.46 (t, J=7.8 Hz, 1H), 7.27-7.21 (m, 3H), 7.12-7.10 (m, 2H), 6.86 (s, 1H), 6.75 (s, 1H), 6.26 (s, 1H), 6.19 (dd, J=16.6, 2.3 Hz, 1H), 5.78 (dd, J=10.4, 2.3 Hz, 1H), 4.98-4.79 (m, 1H), 4.43-4.00 (m, 7H), 3.56-3.39 (m, 3H), 3.19 (s, 3H), 3.00-2.93 (m, 8H), 2.56 (s, 1H), 2.33-2.28 (m, 10H), 2.02-1.97 (m, 1H), 1.54 (s, 1H), 1.37 (s, 8H), 1.25-1.23 (m, 1H), 0.93 (d, J=6.9 Hz, 6H). LC-MS: m/z 1027.4 [M+H]⁺.

### Compound 249

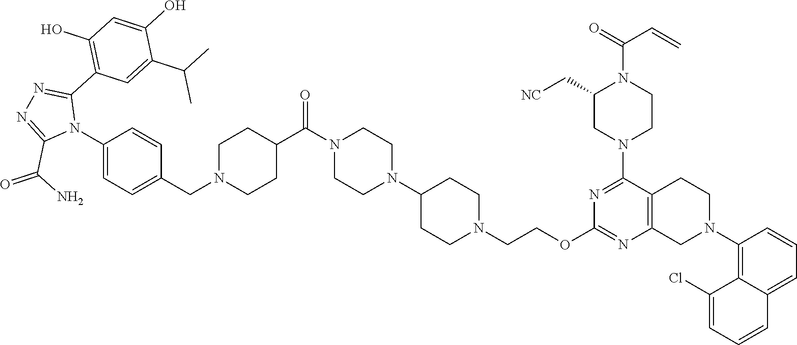 [SMILES: C=CC(=O)N1CCN(c2nc(OCCN3CCC(N4CCN(C(=O)C5CCN(Cc6ccc(-n7c(C(N)=O)nnc7-c7cc(C(C)C)c(O)cc7O)cc6)CC5)CC4)CC3)nc3c2CCN(c2cccc4cccc(Cl)c24)C3)C[C@@H]1CC#N]

### (S)-4-(4-((4-(4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidin-4-yl)piperazine-1-carbonyl)piperidin-1-yl)methyl)phenyl)-5-(2,4-dihydroxy-5-isopropylphenyl)-4H-1,2,4-triazole-3-carboxamide

¹H NMR (400 MHz, MeOD) δ 7.82 (d, J=8.4 Hz, 1H), 7.67 (s, 1H), 7.50 (dd, J=22.9, 7.9 Hz, 4H), 7.39-7.30 (m, 4H), 6.67 (s, 2H), 6.29 (d, J=16.7 Hz, 2H), 5.84 (s, 1H), 4.58 (s, 4H), 4.50 (s, 2H), 4.30 (d, J=17.8 Hz, 2H), 4.16 (s, 2H), 3.70 (s, 1H), 3.58 (s, 6H), 3.22-3.20 (m, 1H), 3.13 (s, 2H), 2.97 (m, 2H), 2.92-2.88 (m, 1H), 2.82 (s, 2H), 2.68 (s, 1H), 2.60 (s, 2H), 2.55 (s, 2H), 2.32 (s, 1H), 2.21 (s, 2H), 2.12 (s, 2H), 2.03 (s, 1H), 1.87 (s, 2H), 1.79 (m, 2H), 1.70 (s, 2H), 1.58 (m, 2H), 1.29 (s, 2H), 1.17 (m, 2H), 0.87 (d, J=6.9 Hz, 6H). LC-MS: m/z 1145.6 [M+H]⁺.

### Compound 250

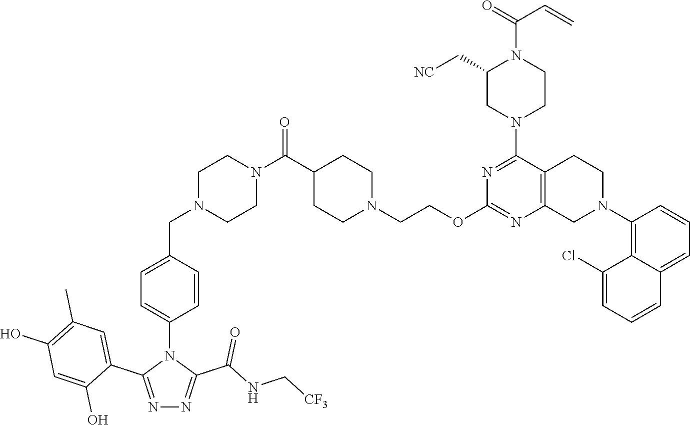 [SMILES: C=CC(=O)N1CCN(c2nc(OCCN3CCC(C(=O)N4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@@H]1CC#N]

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) δ 7.81 (d, J=7.7 Hz, 1H), 7.67 (d, J=5.6 Hz, 1H), 7.53-7.25 (m, 8H), 6.72 (d, J=74.0 Hz, 2H), 6.33-6.20 (m, 2H), 5.81 (s, 1H), 4.49 (s, 3H), 4.30 (m, 2H), 4.15 (s, 2H), 3.98 (m, 3H), 3.70 (s, 2H), 3.60 (s, 6H), 3.19 (d, 3H), 3.09 (s, 3H), 2.91 (s, 1H), 2.80 (s, 2H), 2.66 (s, 2H), 2.46 (d, 4H), 2.22 (m, 2H), 1.87 (s, 3H), 1.80-1.68 (m, 4H), 1.28 (s, 1H). LC-MS: m/z 1116.5 [M+H]⁺.

### Compound 251

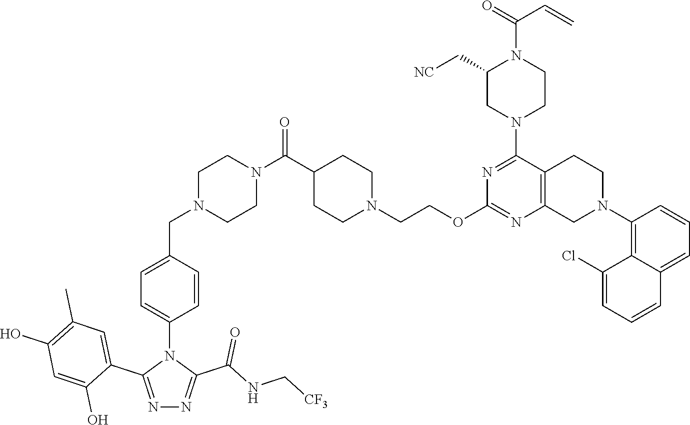 [SMILES: C=CC(=O)N1CCN(c2nc(OCCN3CCC(C(=O)N4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@@H]1CC#N]

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) δ 7.82 (d, J=7.9 Hz, 1H), 7.67 (d, J=8.2 Hz, 1H), 7.55-7.41 (m, 4H), 7.39-7.28 (m, 2H), 7.24 (d, J=8.3 Hz, 2H), 6.73 (s, 2H), 6.28 (d, J=19.1 Hz, 2H), 5.83 (d, J=11.0 Hz, 1H), 4.62-4.45 (m, 5H), 4.21 (m, 5H), 3.75-3.52 (m, 5H), 3.20 (m, 2H), 3.08 (s, 4H), 3.03-2.99 (m, 1H), 2.91 (m, 1H), 2.81 (s, 2H), 2.68 (m, 7H), 2.52 (s, 4H), 2.26 (s, 2H), 2.01-1.64 (m, 7H), 1.29 (s, 3H), 0.91 (d, J=6.9 Hz, 6H). LC-MS: m/z 1118.6 [M+H]⁺.

### Compound 252

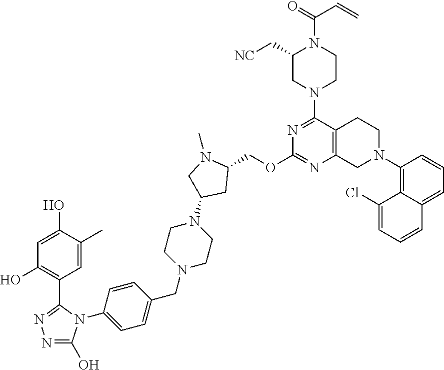 [SMILES: C=CC(=O)N1CCN(c2nc(OC[C@@H]3C[C@H](N4CCN(Cc5ccc(-n6c(O)nnc6-c6cc(C)c(O)cc6O)cc5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-[4-({4-[3-(2,4-dihydroxy-5-methylphenyl)-5-hydroxy-1,2,4-triazol-4-yl]phenyl}methyl)piperazin-1-yl]-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.81 (s, 1H), 9.53 (s, 1H), 9.30 (s, 1H), 8.01-7.82 (m, 1H), 7.78-7.70 (m, 1H), 7.61-7.50 (m, 2H), 7.51 (s, 1H), 7.39-7.29 (m, 1H), 7.28-7.16 (m, 2H), 7.12-7.05 (m, 2H), 6.90-6.86 (m, 2H), 6.81 (s, 1H), 6.21 (d, J=8.2 Hz, 1H), 6.16 (d, J=2.1 Hz, 1H), 5.83-5.72 (m, 1H), 5.05-4.76 (m, 1H), 4.65-3.90 (m, 6H), 3.88-3.69 (m, 2H), 3.60-3.55 (m, 1H), 3.54-3.37 (m, 2H), 3.21-3.00 (m, 4H), 2.99-2.95 (m, 2H), 2.93-2.85 (m, 2H), 2.60-2.55 (m, 2H), 2.44-2.32 (m, 7H), 2.30-2.26 (d, J=3.8 Hz, 4H), 2.11-2.03 (m, 1H), 1.94 (s, 3H), 1.60-1.44 (m, 1H). LC-MS: m/z 965.1 [M+H]⁺.

### Compound 253

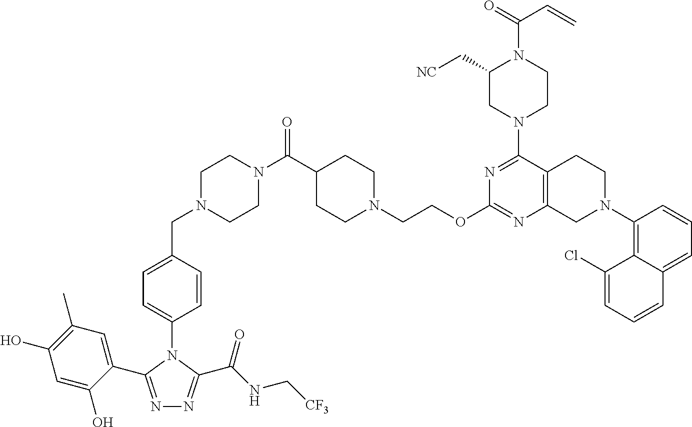 [SMILES: C=CC(=O)N1CCN(c2nc(OCCN3CCC(C(=O)N4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@@H]1CC#N]

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) (400 MHz, MeOD) δ 7.83-7.74 (m, 1H), 7.65 (dd, J=16.4, 8.3 Hz, 1H), 7.55-7.38 (m, 3H), 7.38-7.20 (m, 4H), 7.15-7.07 (m, 1H), 6.89-6.47 (m, 2H), 6.28 (d, J=10.1 Hz, 2H), 5.82 (d, J=10.4 Hz, 1H), 4.67 (s, 1H), 4.45 (s, 1H), 4.31 (m, 2H), 4.17 (m, 1H), 4.05 (s, 1H), 4.03-3.87 (m, 3H), 3.81-3.33 (m, 7H), 3.22-3.03 (m, 4H), 3.02-2.78 (m, 5H), 2.66 (m, 3H), 2.44 (m, 3H), 2.38-1.90 (m, 4H), 1.69 (m, 7H), 1.31 (m, 1H). LC-MS: m/z 1116.4 [M+H]⁺.

### Compound 254

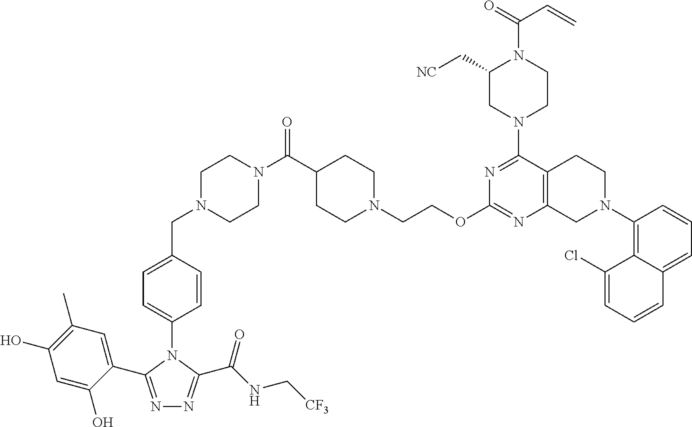 [SMILES: C=CC(=O)N1CCN(c2nc(OCCN3CCC(C(=O)N4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@@H]1CC#N]

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) δ 7.80 (s, 1H), 7.65 (d, J=8.0 Hz, 1H), 7.49 (d, J=20.5 Hz, 3H), 7.38-7.27 (m, 4H), 7.20 (d, J=7.3 Hz, 1H), 6.91-6.55 (m, 2H), 6.31 (dd, J=18.4, 11.3 Hz, 2H), 5.83 (d, J=9.3 Hz, 1H), 4.73-4.63 (m, 2H), 4.46 (s, 1H), 4.28 (s, 2H), 4.17 (s, 1H), 4.07 (s, 1H), 3.96 (s, 1H), 3.68 (m, 2H), 3.58 (s, 2H), 3.48 (s, 2H), 3.41 (s, 3H), 3.15 (m, 4H), 2.96 (s, 3H), 2.86 (s, 2H), 2.67 (s, 4H), 2.54 (m, 6H), 2.45 (s, 5H), 2.29 (m, 4H), 2.17 (s, 1H), 2.02 (s, 1H), 1.72 (s, 3H), 1.60 (s, 1H), 1.31 (m, 4H), 0.85 (m, 6H). LC-MS: m/z 1188.5 [M+H]⁺.

### Compound 255

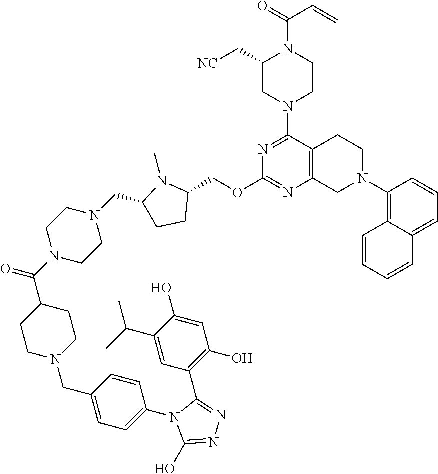 [SMILES: C=CC(=O)N1CCN(c2nc(OC[C@@H]3CC[C@H](CN4CCN(C(=O)C5CCN(Cc6ccc(-n7c(O)nnc7-c7cc(C(C)C)c(O)cc7O)cc6)CC5)CC4)N3C)nc3c2CCN(c2cccc4ccccc24)C3)C[C@@H]1CC#N]

### 2-[(2S)-4-(2-{[(2S,5R)-5-({4-[1-({4-[3-(2,4-dihydroxy-5-isopropylphenyl)-5-hydroxy-1,2,4-triazol-4-yl]phenyl}methyl)piperidine-4-carbonyl]piperazin-1-yl} methyl)-1-methylpyrrolidin-2-yl]methoxy}-7-(naphthalen-1-yl)-5H,6H,8H-pyri-do[3,4-d]pyrimidin-4-yl)-1-(prop-2-enoyl) piperazin-2-yl] acetonitrile

¹H NMR (DMSO-d₆, 400 MHz): δ 11.93 (s, 1H), 9.59 (s, 1H), 9.41 (s, 1H), 8.22-8.15 (m, 1H), 7.96-7.89 (m, 1H), 7.64 (d, J=8.2 Hz, 1H), 7.58-7.50 (m, 2H), 7.46 (t, J=7.8 Hz, 1H), 7.25 (m, 3H), 7.12 (d, J=8.1 Hz, 2H), 6.87 (s, 1H), 6.76 (s, 1H), 6.26 (s, 1H), 6.19 (m, 1H), 5.78 (d, J=11.2 Hz, 1H), 5.08-4.72 (m, 1H), 4.51-4.10 (m, 4H), 4.04 (s, 3H), 3.57 (s, 1H), 3.41 (s, 7H), 3.18 (s, 3H), 2.99 (d, J=6.8 Hz, 5H), 2.93 (d, J=6.7 Hz, 2H), 2.78 (d, J=10.6 Hz, 1H), 2.70 (s, 2H), 2.45-2.28 (m, 8H), 2.25-2.16 (m, 1H), 1.95 (s, 2H), 1.86 (s, 2H), 1.54 (s, 6H), 0.93 (d, J=6.9 Hz, 6H). LC-MS: m/z 1084.2 [M+H]⁺.

### Compound 256

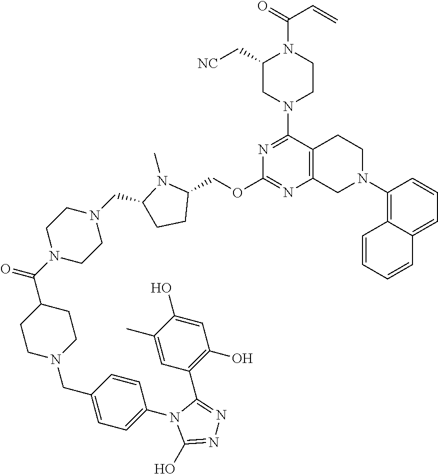 [SMILES: C=CC(=O)N1CCN(c2nc(OC[C@@H]3CC[C@H](CN4CCN(C(=O)C5CCN(Cc6ccc(-n7c(O)nnc7-c7cc(C)c(O)cc7O)cc6)CC5)CC4)N3C)nc3c2CCN(c2cccc4ccccc24)C3)C[C@@H]1CC#N]

### 2-[(2S)-4-(2-{[(2S,5R)-5-({4-[1-({4-[3-(2,4-dihydroxy-5-methylphenyl)-5-hydroxy-1,2,4-triazol-4-yl]phenyl}methyl)piperidine-4-carbonyl]piperazin-1-yl}methyl)-1-methylpyrrolidin-2-yl]methoxy}-7-(naphthalen-1-yl)-5H,6H,8H-pyrido[3,4-d]pyrimidin-4-yl)-1-(prop-2-enoyl)piperazin-2-yl]acetonitrile

¹H NMR (DMSO-d₆, 400 MHz): δ 11.88 (s, 1H), 9.55 (s, 1H), 9.32 (s, 1H), 8.22-8.15 (m, 1H), 7.93 (dd, J=6.8, 3.0 Hz, 1H), 7.64 (d, J=8.2 Hz, 1H), 7.58-7.42 (m, 3H), 7.23 (t, J=7.3 Hz, 3H), 7.09 (d, J=8.0 Hz, 2H), 6.88 (s, 2H), 6.31-6.08 (m, 2H), 5.82-5.74 (m, 1H), 5.06-4.72 (m, 1H), 4.50-4.08 (m, 4H), 4.03 (s, 3H), 3.41 (s, 7H), 3.18 (s, 3H), 3.09-2.97 (m, 4H), 2.94 (s, 2H), 2.76 (d, J=10.6 Hz, 2H), 2.73-2.63 (m, 2H), 2.39 (s, 8H), 2.25-2.17 (m, 1H), 2.09-1.71 (m, 7H), 1.54 (s, 6H). LC-MS: m/z 1056.2 [M+H]⁺.

### Compound 257

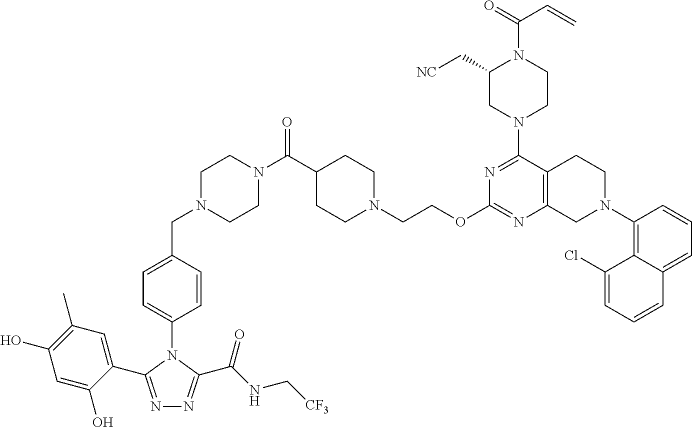 [SMILES: C=CC(=O)N1CCN(c2nc(OCCN3CCC(C(=O)N4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@@H]1CC#N]

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) δ 7.82 (d, J=7.8 Hz, 1H), 7.68 (d, J=8.1 Hz, 1H), 7.55-7.44 (m, 4H), 7.40-7.29 (m, 4H), 6.90-6.73 (m, 1H), 6.67 (s, 1H), 6.29 (d, J=16.9 Hz, 2H), 5.83 (d, J=10.8 Hz, 1H), 4.53-4.26 (m, 5H), 4.22-3.98 (m, 3H), 3.75-3.47 (m, 8H), 3.27-3.08 (m, 7H), 3.02-2.83 (m, 6H), 2.76-2.63 (m, 3H), 2.59-2.44 (m, 1H), 2.14 (m, 2H), 1.90-1.65 (m, 6H), 1.23-1.07 (m, 2H), 0.87 (d, J=6.9 Hz, 6H). LC-MS: m/z 1102.5 [M+H]⁺.

### Compound 258

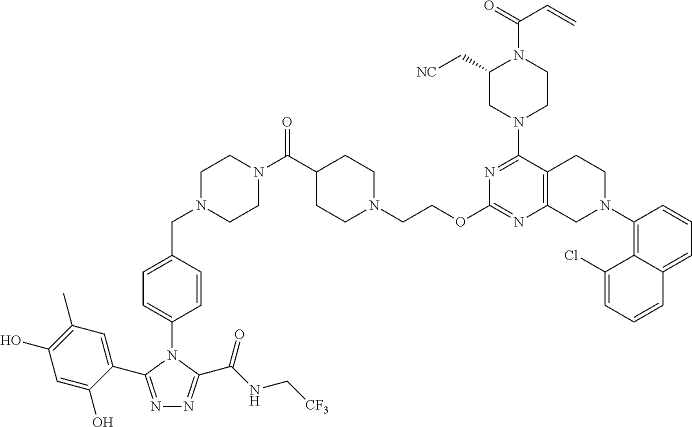 [SMILES: C=CC(=O)N1CCN(c2nc(OCCN3CCC(C(=O)N4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@@H]1CC#N]

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) δ 7.82 (d, J=8.1 Hz, 1H), 7.68 (d, J=7.9 Hz, 1H), 7.50 (dd, J=17.4, 8.0 Hz, 4H), 7.39-7.29 (m, 4H), 6.74 (d, J=51.2 Hz, 2H), 6.28 (d, J=17.1 Hz, 2H), 5.83 (d, J=10.1 Hz, 1H), 4.55 (m, 5H), 4.37-4.00 (m, 5H), 3.71 (m, 2H), 3.60 (m, 7H), 3.48 (s, 1H), 3.20 (m, 5H), 2.98 (m, 4H), 2.90 (s, 3H), 2.77-2.63 (m, 3H), 2.58 (m, 4H), 2.32 (s, 3H), 2.17 (m, 2H), 1.91 (m, 2H), 1.79 (m, 2H), 1.70 (m, 2H), 1.61 (m, 2H), 1.30 (d, J=7.4 Hz, 1H), 1.15 (t, J=7.3 Hz, 3H), 0.87 (d, J=6.9 Hz, 6H). LC-MS: m/z 1173.6 [M+H]⁺.

### Compound 259

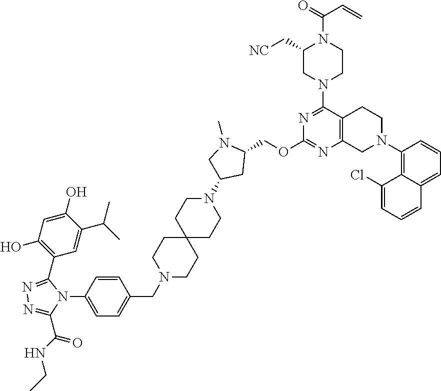 [SMILES: C=CC(=O)N1CCN(c2nc(OC[C@@H]3C[C@H](N4CCC5(CCN(Cc6ccc(-n7c(C(=O)NCC)nnc7-c7cc(C(C)C)c(O)cc7O)cc6)CC5)CC4)CN3C)nc3c2CCN(c2cccc4cccc(Cl)c24)C3)C[C@@H]1CC#N]

4-[4-({9-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]-3,9-diazaspiro[5.5]undecan-3-yl}methyl)phenyl]-5-(2,4-di hydroxy-5-isopropylphenyl)-N-ethyl-1,2,4-triazole-3-carboxamide

¹H NMR (DMSO-d₆, 400 MHz): δ 10.59 (s, 1H), 9.76 (s, 1H), 8.96 (t, J=5.9 Hz, 1H), 7.91 (d, J=8.1 Hz, 1H), 7.74 (dd, J=8.2, 3.5 Hz, 1H), 7.59-7.50 (m, 2H), 7.46-7.42 (m, 1H), 7.39-7.31 (m, 4H), 7.28 (d, J=8.0 Hz, 2H), 6.85 (s, 1H), 6.57 (s, 1H), 6.34 (s, 1H), 6.19 (d, J=16.6 Hz, 1H), 5.77 (d, J=10.4 Hz, 1H), 5.06-4.96 (m, 1H), 4.73 (t, J=5.7 Hz, 1H), 4.27-4.14 (m, 3H), 4.01-3.86 (m, 2H), 3.78-3.69 (m, 2H), 3.46-3.42 (m, 3H), 3.16 (p, J=7.1 Hz, 3H), 3.10-3.05 (m, 4H), 2.93-2.86 (m, 3H), 2.34-2.28 (m, 10H), 2.07 (s, 2H), 1.91-1.84 (m, 2H), 1.40 (s, 9H), 1.24 (s, 1H), 1.03 (t, J=7.2 Hz, 3H), 0.80 (d, J=6.9 Hz, 6H). LC-MS: m/z 1116.2 [M+H]⁺.

### Compound 260

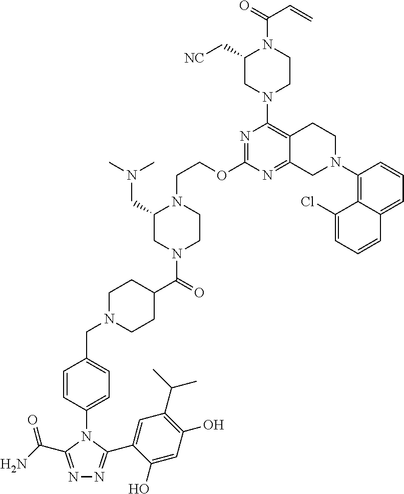 [SMILES: C=CC(=O)N1CCN(c2nc(OCCN3CCN(C(=O)C4CCN(Cc5ccc(-n6c(C(N)=O)nnc6-c6cc(C(C)C)c(O)cc6O)cc5)CC4)C[C@@H]3CN(C)C)nc3c2CCN(c2cccc4cccc(Cl)c24)C3)C[C@@H]1CC#N]

### 4-(4-((4-((S)-4-(2-((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)-3-((dimethylamino)methyl)piperazine-1-carbonyl)piperidin-1-yl)methyl)phenyl)-5-(2,4-dihydroxy-5-isopropylphenyl)-4H-1,2,4-triazole-3-carboxamide

¹H NMR (400 MHz, MeOD) δ 7.85 (d, J=7.8 Hz, 1H), 7.72 (d, J=8.0 Hz, 1H), 7.64-7.35 (m, 8H), 6.93-6.67 (m, 2H), 6.31 (d, J=15.6 Hz, 2H), 5.85 (d, J=10.3 Hz, 1H), 4.69-4.52 (m, 3H), 4.44-4.22 (m, 5H), 4.19-4.01 (m, 2H), 3.82-3.70 (m, 2H), 3.66-3.47 (m, 6H), 3.44-3.35 (m, 3H), 3.27-3.18 (m, 3H), 3.15-3.02 (m, 7H), 3.01-2.86 (m, 9H), 2.07-1.97 (m, 3H), 1.35-1.27 (m, 3H), 0.93 (d, J=6.9 Hz, 6H). LC-MS: m/z 1119.5 [M+H]⁺.

### Compound 261

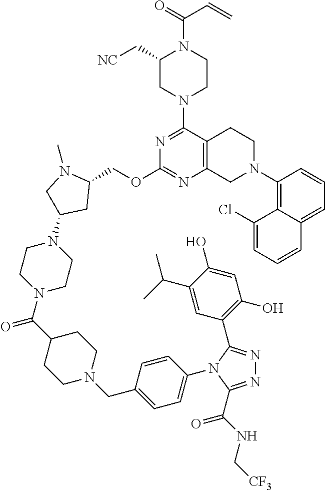 [SMILES: C=CC(=O)N1CCN(c2nc(OC[C@@H]3C[C@H](N4CCN(C(=O)C5CCN(Cc6ccc(-n7c(C(=O)NCC(F)(F)F)nnc7-c7cc(C(C)C)c(O)cc7O)cc6)CC5)CC4)CN3C)nc3c2CCN(c2cccc4cccc(Cl)c24)C3)C[C@@H]1CC#N]

### 4-{4-[(4-{4-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}meth yl)-1-methylpyrrolidin-3-yl]piperazine-1-carbonyl}piperidin-1-yl)methyl]phenyl}-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.47 (s, 1H), 9.76 (s, 1H), 9.58 (t, J=6.5 Hz, 1H), 7.91 (d, J=8.1 Hz, 1H), 7.74 (t, J=6.4 Hz, 1H), 7.58 (d, J=7.3 Hz, 1H), 7.57-7.53 (m, 1H), 7.44 (t, J=7.8 Hz, 1H), 7.39-7.27 (m, 5H), 6.85 (s, 1H), 6.61 (s, 1H), 6.34 (s, 1H), 6.18 (d, J=16.6 Hz, 1H), 5.77 (d, J=10.6 Hz, 1H), 4.97-4.75 (m, 1H), 4.45-4.12 (m, 5H), 4.05-3.91 (m, 5H), 3.82-3.70 (m, 2H), 3.50-3.48 (m, 3H), 3.15-3.03 (m, 4H), 3.02-2.88 (s, 7H), 2.85-2.73 (m, 4H), 2.42-2.38 (m, 2H), 2.32-2.20 (m, 6H), 2.12-1.90 (m, 4H), 1.65-1.52 (m, 5H), 1.22 (s, 1H), 0.92-0.78 (m, 6H). LC-MS: m/z 1213.1 [M+H]⁺.

### Compound 262

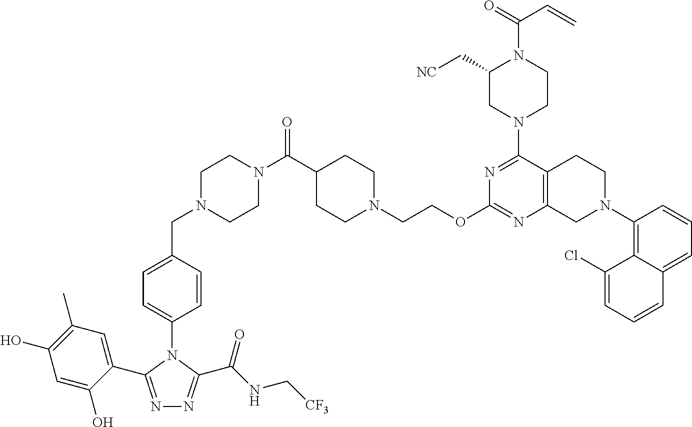 [SMILES: C=CC(=O)N1CCN(c2nc(OCCN3CCC(C(=O)N4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@@H]1CC#N]

### (S)-4-(4-((4-(1-(2-((4-(4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)piperidine-4-carbonyl)piperazin-1-yl)methyl)phenyl)-5-(2,4-dihydroxy-5-methylphenyl)-N-(2,2,2-trifluoroethyl)-4H-1,2,4-triazole-3-carboxamide

¹H NMR (400 MHz, MeOD) δ 7.82 (d, J=8.0 Hz, 1H), 7.67 (d, J=8.2 Hz, 1H), 7.57-7.42 (m, 4H), 7.41-7.26 (m, 4H), 6.95-6.72 (m, 1H), 6.29 (d, J=19.8 Hz, 2H), 5.83 (d, J=9.9 Hz, 1H), 4.52 (m, 6H), 4.36-4.25 (m, 2H), 4.23-3.92 (m, 6H), 3.76-3.47 (m, 8H), 3.27-3.05 (m, 7H), 3.04-2.88 (m, 6H), 2.80-2.60 (m, 3H), 2.57-2.39 (m, 1H), 2.20-2.05 (m, 2H), 1.92-1.74 (m, 4H), 1.68 (m, 2H), 1.39-1.22 (m, 2H), 1.22-1.02 (m, 3H), 0.87 (d, J=6.9 Hz, 6H). LC-MS: m/z 1184.5 [M+H]⁺.

### Compound 263

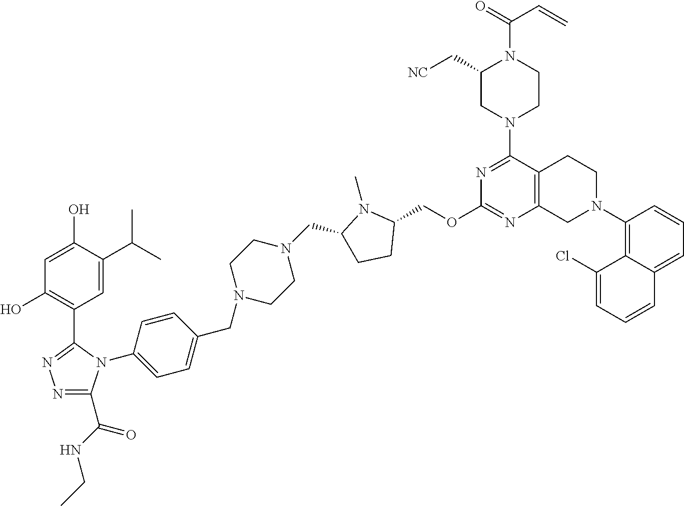 [SMILES: C=CC(=O)N1CCN(c2nc(OC[C@@H]3CC[C@H](CN4CCN(Cc5ccc(-n6c(C(=O)NCC)nnc6-c6cc(C(C)C)c(O)cc6O)cc5)CC4)N3C)nc3c2CCN(c2cccc4cccc(Cl)c24)C3)C[C@@H]1CC#N]

### 4-{4-[(4-{[(2R,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-2-yl]methyl}piperazin-1-yl)methyl]phenyl}-5-(2,4-dihydroxy-5-isopropylphenyl)-N-ethyl-1,2,4-triazole-3-carboxamide

¹H NMR (DMSO-d₆, 400 MHz): δ 10.59 (s, 1H), 9.74 (s, 1H), 8.94 (t, J=5.9 Hz, 1H), 7.92 (d, J=8.1 Hz, 1H), 7.78-7.70 (m, 1H), 7.54 (dt, J=16.1, 7.9 Hz, 2H), 7.44 (td, J=7.8, 1.4 Hz, 1H), 7.36 (d, J=8.1 Hz, 3H), 7.28 (d, J=8.3 Hz, 2H), 6.85 (s, 1H), 6.57 (s, 1H), 6.34 (s, 1H), 6.18 (d, J=16.6 Hz, 1H), 5.81-5.73 (m, 1H), 5.06-4.71 (m, 1H), 4.52-3.90 (m, 6H), 3.89-3.67 (m, 2H), 3.47 (s, 3H), 3.23-2.78 (m, 10H), 2.38 (s, 12H), 2.22-2.15 (m, 2H), 1.87 (s, 3H), 1.57-1.42 (m, 2H), 1.24 (s, 1H), 1.03 (t, J=7.2 Hz, 3H), 0.80 (d, J=6.9 Hz, 6H). LC-MS: m/z 1062.2 [M+H]⁺.

### Compound 264

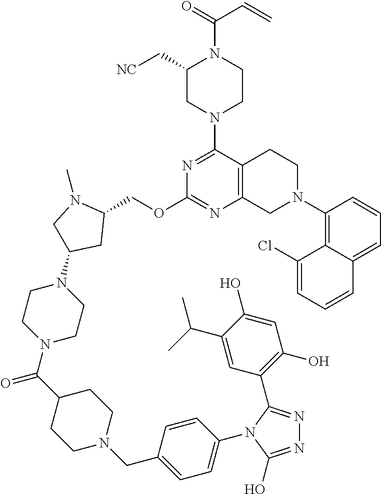 [SMILES: C=CC(=O)N1CCN(c2nc(OC[C@@H]3C[C@H](N4CCN(C(=O)C5CCN(Cc6ccc(-n7c(O)nnc7-c7cc(C(C)C)c(O)cc7O)cc6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{4-[1-({4-[3-(2,4-dihydroxy-5-isopropylphenyl)-5-hydroxy-1,2,4-triazol-4-yl]phenyl}methyl)piperidine-4-carbonyl]piperazin-1-yl}-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 10.04 (s, 1H), 9.61 (s, 1H), 7.95-7.88 (m, 1H), 7.74 (dd, J=4.2 Hz, 1H), 7.61-7.48 (m, 2H), 7.44 (t, J=7.8 Hz, 1H), 7.34 (dd, J=7.4 Hz, 1H), 7.17 (s, 3H), 7.04 (s, 1H), 6.85 (s, 1H), 6.39 (s, 1H), 6.18 (d, J=16.5 Hz, 1H), 5.77 (d, J=10.7 Hz, 1H), 4.96-4.75 (s, 5H), 4.27 (d, J=8.9 Hz, 1H), 4.19 (d, J=15.7 Hz, 3H), 4.01 (d, J=14.2 Hz, 2H), 3.49 (s, 2H), 3.39 (s, 1H), 3.30 (s, 2H), 3.08 (q, J=6.9 Hz, 5H), 2.97 (s, 2H), 2.91-2.87 (m, 3H), 2.48 (d, J=2.7 Hz, 1H), 2.29-2.23 (m, 9H), 2.02 (s, 2H), 1.53 (s, 1H), 1.36 (s, 9H), 1.13 (d, J=6.9 Hz, 6H). LC-MS: m/z 1104.1 [M+H]⁺.

### Compound 265

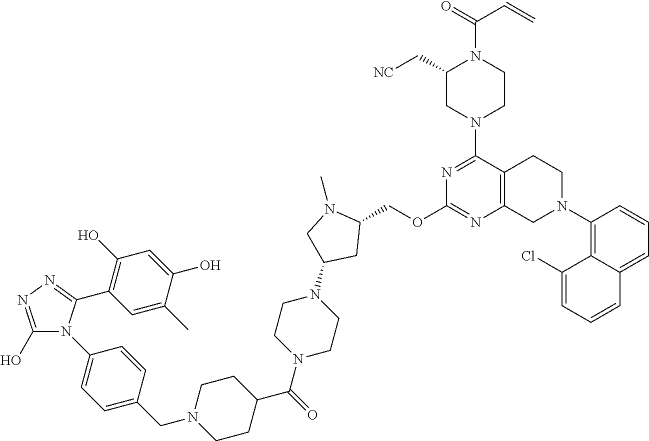 [SMILES: C=CC(=O)N1CCN(c2nc(OC[C@@H]3C[C@H](N4CCN(C(=O)C5CCN(Cc6ccc(-n7c(O)nnc7-c7cc(C)c(O)cc7O)cc6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{4-[1-({4-[3-(2,4-dihydroxy-5-methylphenyl)-5-hydroxy-1,2,4-triazol-4-yl]phenyl}methyl)piperidine-4-carbon yl]piperazin-1-yl}-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d, 400 MHz): δ 11.88 (s, 1H), 9.56 (s, 1H), 9.32 (s, 1H), 7.92 (d, J=8.2 Hz, 1H), 7.85-7.70 (in, 1H), 7.68-7.52 (m, 2H), 7.45 (t, J=7.8 Hz, 1H), 7.38-7.30 (m, 1H), 7.24 (d, J=7.9 Hz, 2H), 7.09 (d, J=8.0 Hz, 2H), 6.88 (s, 2H), 6.31-6.05 (m, 2H), 5.81-5.73 (m, 1H), 5.02-4.56 (m, 1H), 4.52-3.61 (m, 8H), 3.53-3.35 (m, 8H), 3.22-2.84 (s, 8H), 2.82-2.70 (m, 2H), 2.41-2.13 (m, 9H), 2.10-1.86 (m, 6H), 1.70-1.44 (s, 5H), 1.39 (s, 1H), 1.28-1.25 (m, 1H), 0.85-0.78 (m, 1H). LC-MS: m/z 1076.1 [M+H]⁺.

### Compound 266

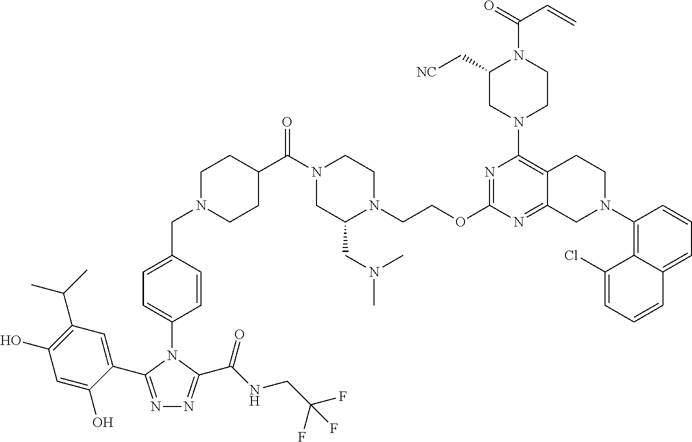 [SMILES: C=CC(=O)N1CCN(c2nc(OCCN3CCN(C(=O)C4CCN(Cc5ccc(-n6c(C(=O)NCC(F)(F)F)nnc6-c6cc(C(C)C)c(O)cc6O)cc5)CC4)C[C@H]3CN(C)C)nc3c2CCN(c2cccc4cccc(Cl)c24)C3)C[C@@H]1CC#N]

### 4-(4-((4-((R)-4-(2-((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)ethyl)-3-((dimethylamino)methyl)piperazine-1-carbonyl)piperidin-1-yl)methyl)phenyl)-5-(2,4-dihydroxy-5-isopropylphenyl)-N-(2,2,2-trifluoroethyl)-4H-1,2,4-triazole-3-carboxamide

¹H NMR (400 MHz, MeOD) δ 7.84 (d, J=7.9 Hz, 1H), 7.70 (d, J=8.0 Hz, 1H), 7.62 (d, J=8.3 Hz, 2H), 7.51 (dd, J=16.8, 7.8 Hz, 4H), 7.44-7.28 (m, 2H), 6.88-6.72 (m, 2H), 6.29 (d, J=17.1 Hz, 2H), 5.84 (d, J=10.8 Hz, 1H), 4.54-4.48 (m, 2H), 4.41-4.24 (m, 4H), 4.10-3.92 (m, 4H), 3.81-3.66 (m, 3H), 3.65-3.53 (m, 6H), 3.49-3.38 (m, 3H), 3.25-3.17 (m, 3H), 3.13-3.00 (m, 6H), 2.97-2.88 (m, 8H), 2.79-2.68 (m, 1H), 2.07-1.97 (m, 3H), 1.34-1.26 (m, 4H), 0.94 (d, J=6.9 Hz, 6H). LC-MS: m/z 1201.5 [M+H]⁺.

### Compound 267

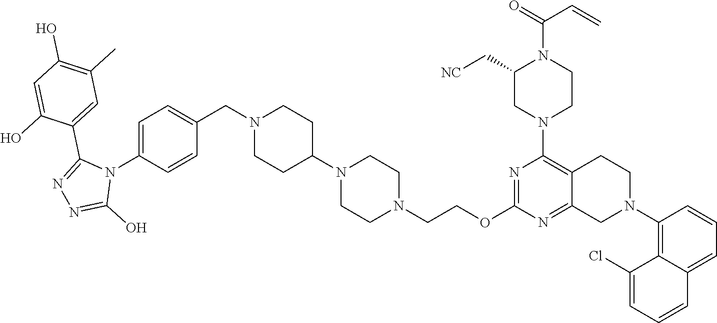 [SMILES: C=CC(=O)N1CCN(c2nc(OCCN3CCN(C4CCN(Cc5ccc(-n6c(O)nnc6-c6cc(C)c(O)cc6O)cc5)CC4)CC3)nc3c2CCN(c2cccc4cccc(Cl)c24)C3)C[C@@H]1CC#N]

### (S)-2-(1-acryloyl-4-(7-(8-chloronaphthalen-1-yl)-2-(2-(4-(1-(4-(3-(2,4-dihydroxy-5-methylphenyl)-5-hydroxy-4H-1,2,4-triazol-4-yl)benzyl)piperidin-4-yl)piperazin-1-yl)ethoxy)-5,6,7,8-tetrahydropyrido[3,4-d]pyrimidin-4-yl)piperazin-2-yl)acetonitrile

¹H NMR (400 MHz, MeOD) δ 7.82 (d, J=7.9 Hz, 1H), 7.70-7.64 (m, 1H), 7.51 (d, J=16.1, 7.4 Hz, 2H), 7.41-7.29 (m, 4H), 7.22 (d, J=8.1 Hz, 2H), 6.77 (s, 2H), 6.33-6.16 (m, 2H), 5.83 (d, J=9.9 Hz, 1H), 4.36-4.26 (m, 2H), 4.22-3.95 (m, 4H), 3.82-3.51 (m, 7H), 3.24-3.11 (m, 4H), 3.01-2.87 (m, 4H), 2.84-2.81 (m, 2H), 2.72-2.67 (m, 4H), 2.37-2.32 (m, 1H), 2.19 (t, J=7.6 Hz, 3H), 2.05-2.03 (m, 2H), 1.96-1.93 (m, 3H), 1.61-1.58 (m, 2H), 0.91-0.88 (m, 3H). LC-MS: m/z 979.5 [M+H]⁺.

### Compound 268

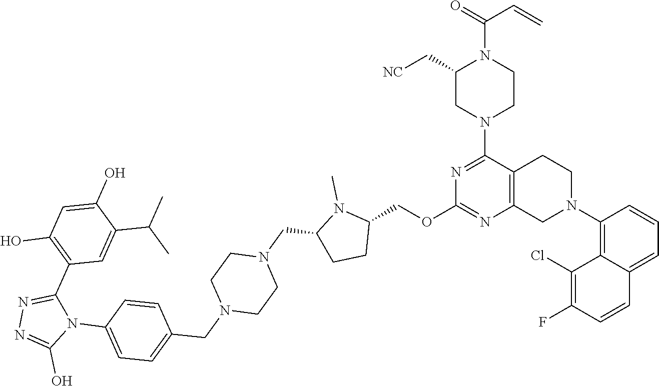 [SMILES: C=CC(=O)N1CCN(c2nc(OC[C@@H]3CC[C@H](CN4CCN(Cc5ccc(-n6c(O)nnc6-c6cc(C(C)C)c(O)cc6O)cc5)CC4)N3C)nc3c2CCN(c2cccc4ccc(F)c(Cl)c24)C3)C[C@@H]1CC#N]

### 2-[(2S)-4-[7-(8-chloro-7-fluoronaphthalen-1-yl)-2-{[(2S,5R)-5-{[4-({4-[3-(2,4-dihydroxy-5-isopropylphenyl)-5-hydroxy-1,2,4-triazol-4-yl]phenyl}methyl)piperazin-1-yl]methyl}-1-methylpyrrolidin-2-yl]methoxy}-5H,6H,8H-pyrido[3,4-d]pyramid in-4-yl]-1-(prop-2-enoyl)piperazin-2-yl]acetonitrile

¹H NMR (DMSO-d₆, 400 MHz): δ 11.92 (s, 1H), 9.58 (s, 1H), 9.40 (s, 1H), 8.02 (dd, J=9.0, 5.8 Hz, 1H), 7.79 (dd, J=8.1, 4.2 Hz, 1H), 7.61-7.50 (m, 2H), 7.45-7.38 (m, 1H), 7.27 (d, J=7.9 Hz, 2H), 7.12 (d, J=8.0 Hz, 2H), 6.85 (s, 1H), 6.75 (s, 1H), 6.26 (s, 1H), 6.18 (d, J=16.6 Hz, 1H), 5.77 (d, J=10.6 Hz, 1H), 4.95-4.76 (m, 1H), 4.18-3.72 (m, 7H), 3.41 (s, 3H), 3.15-2.85 (m, 9H), 2.67 (p, J=1.9 Hz, 1H), 2.36-2.32 (m, 12H), 2.17 (d, J=8.3 Hz, 2H), 1.83 (s, 2H), 1.51-1.44 (m, 2H), 1.24 (s, 1H), 0.93 (d, J=6.9 Hz, 6H). LC-MS: m/z 1025.1 [M+H]⁺.

### Compound 269

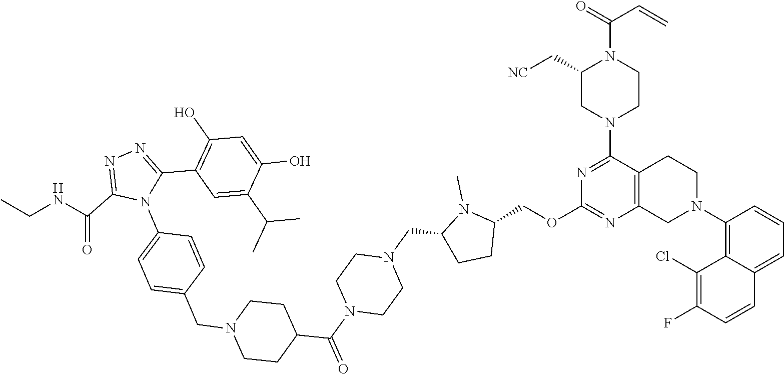 [SMILES: C=CC(=O)N1CCN(c2nc(OC[C@@H]3CC[C@H](CN4CCN(C(=O)C5CCN(Cc6ccc(-n7c(C(=O)NCC)nnc7-c7cc(C(C)C)c(O)cc7O)cc6)CC5)CC4)N3C)nc3c2CCN(c2cccc4ccc(F)c(Cl)c24)C3)C[C@@H]1CC#N]

### 4-(4-{[4-(4-{[(2R,5S)-5-({[7-(8-chloro-7-fluoronaphthalen-1-yl)-4-[(3S)-3-(cyano methyl)-4-(prop-2-enoyl)piperazin-1-yl]-5H,6H,8H-pyrido[3,4-d]pyrimidin-2-yl]oxy}methyl)-1-methylpyrrolidin-2-yl]methyl}piperazine-1-carbonyl)piperidin-1-yl]methyl}phenyl)-5-(2,4-dihydroxy-5-isopropylphenyl)-N-ethyl-1,2,4-triazole-3-carboxamide

¹H NMR (DMSO-d₆, 400 MHz): δ 10.62 (s, 1H), 9.75 (s, 1H), 8.95 (t, J=5.9 Hz, 1H), 8.02 (dd, J=9.1, 5.8 Hz, 1H), 7.79 (dd, J=8.1, 4.3 Hz, 1H), 7.61 (d, J=8.9 Hz, 1H), 7.53 (q, J=8.2 Hz, 1H), 7.46-7.36 (m, 3H), 7.30-7.28 (m, 2H), 6.83 (s, 1H), 6.57 (s, 1H), 6.34 (s, 1H), 6.18 (d, J=16.6 Hz, 1H), 5.77 (d, J=11.2 Hz, 1H), 4.95-4.77 (m, 1H), 4.41-3.62 (m, 7H), 3.47-3.40 (m, 7H), 3.20-3.05 (m, 8H), 2.91 (q, J=6.9 Hz, 1H), 2.84-2.81 (m, 2H), 2.68-2.66 (m, 2H), 2.42-2.32 (m, 9H), 2.21-2.17 (m, 1H), 1.99 (s, 2H), 1.84 (d, J=7.2 Hz, 3H), 1.56 (s, 6H), 1.03 (t, J=7.2 Hz, 3H), 0.80 (d, J=6.9 Hz, 6H). LC-MS: m/z 1191.2 [M+H]⁺.

### Compound 270

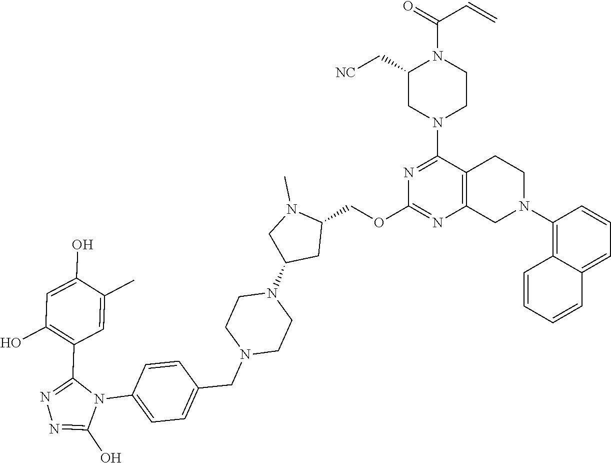 [SMILES: C=CC(=O)N1CCN(c2nc(OC[C@@H]3C[C@H](N4CCN(Cc5ccc(-n6c(O)nnc6-c6cc(C)c(O)cc6O)cc5)CC4)CN3C)nc3c2CCN(c2cccc4ccccc24)C3)C[C@@H]1CC#N]

### 2-[(2S)-4-(2-{[(2S,4S)-4-[4-({4-[3-(2,4-dihydroxy-5-methylphenyl)-5-hydroxy-1,2,4-triazol-4-yl]phenyl}methyl)piperazin-1-yl]-1-methylpyrrolidin-2-yl]methoxy}-7-(naphthalen-1-yl)-5H,6H,8H-pyrido[3,4-d]pyrimidin-4-yl)-1-(prop-2-enoyl)piperazin-2-yl]acetonitrile

¹H NMR (DMSO-d₆, 400 MHz): δ 11.86 (s, 1H), 9.54 (s, 1H), 9.30 (s, 1H), 8.22-8.15 (m, 1H), 7.99-7.84 (m, 1H), 7.64 (d, J=8.2 Hz, 1H), 7.56-7.44 (m, 3H), 7.25-7.21 (m, 3H), 7.09-7.07 (m, 2H), 6.88 (s, 2H), 6.22 (d, J=3.8 Hz, 1H), 6.17 (d, J=2.4 Hz, 1H), 5.78 (dd, J=10.4, 2.3 Hz, 1H), 4.97-4.77 (m, 1H), 4.59-3.86 (m, 7H), 3.40 (s, 3H), 3.25-2.78 (m, 10H), 2.68-2.59 (m, 1H), 2.47-2.19 (m, 12H), 2.13-2.01 (m, 1H), 1.94 (s, 3H), 1.53 (d, J=10.5 Hz, 1H). LC-MS: m/z 931.2 [M+H]⁺.

### Compound 271

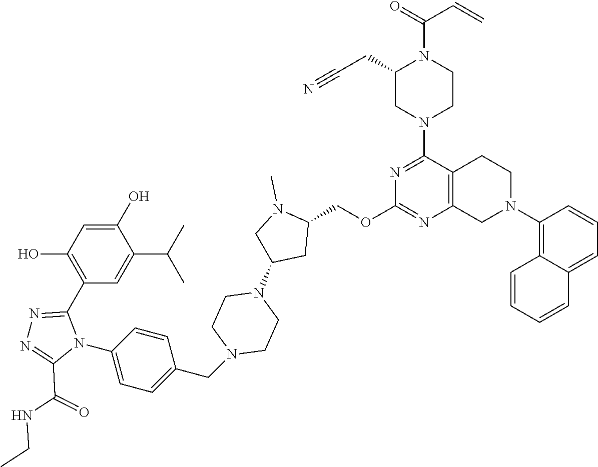 [SMILES: C=CC(=O)N1CCN(c2nc(OC[C@@H]3C[C@H](N4CCN(Cc5ccc(-n6c(C(=O)NCC)nnc6-c6cc(C(C)C)c(O)cc6O)cc5)CC4)CN3C)nc3c2CCN(c2cccc4ccccc24)C3)C[C@@H]1CC#N]

### 4-[4-({4-[(3S,5S)-5-[({4-[(3S)-3-(cyanomethyl)-4-(prop-2-enoyl)piperazin-1-yl]-7-(naphthalen-1-yl)-5H,6H,8H-pyrido[3,4-d]pyrimidin-2-yl}oxy)methyl]-1-methyl pyrrolidin-3-yl]piperazin-1-yl}methyl)phenyl]-5-(2,4-dihydroxy-5-isopropylphenyl)-N-ethyl-1,2,4-triazole-3-carboxamide

¹H NMR (DMSO-d₆, 400 MHz): δ 10.59 (s, 1H), 9.74 (s, 1H), 8.95 (t, J=5.9 Hz, 1H), 8.22-8.15 (m, 1H), 7.93-7.91 (m, 1H), 7.64 (d, J=8.2 Hz, 1H), 7.57-7.51 (m, 2H), 7.46 (t, J=7.8 Hz, 1H), 7.35 (d, J=8.0 Hz, 2H), 7.27 (d, J=8.3 Hz, 2H), 7.23 (d, J=7.4 Hz, 1H), 6.86 (s, 1H), 6.57-6.52 (m, 1H), 6.34 (s, 1H), 6.21-6.17 (m, 1H), 5.79-5.76 (m, 1H), 5.06-4.65 (m, 1H), 4.51-4.27 (m, 2H), 4.24-4.13 (m, 3H), 4.11-3.91 (m, 3H), 3.47 (s, 3H), 3.16 (p, J=7.0 Hz, 5H), 3.11-2.84 (m, 8H), 2.67 (p, J=1.9 Hz, 1H), 2.57 (d, J=8.2 Hz, 5H), 2.39 (s, 4H), 2.14-2.01 (m, 1H), 1.55 (d, J=6.0 Hz, 1H), 1.24 (s, 2H), 1.03 (t, J=7.2 Hz, 3H), 0.80 (d, J=6.9 Hz, 6H). LC-MS: m/z 1014.4 [M+H]⁺.

### Compound 272

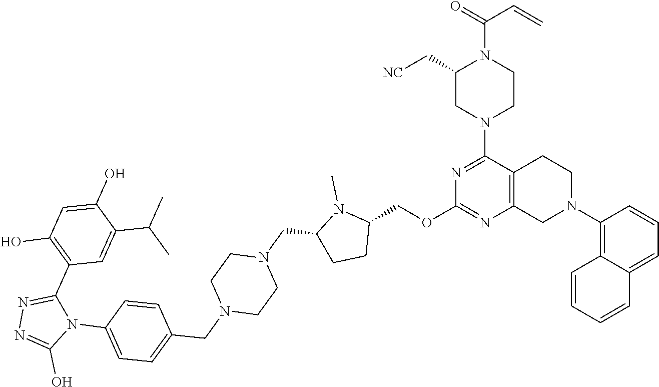 [SMILES: C=CC(=O)N1CCN(c2nc(OC[C@@H]3CC[C@H](CN4CCN(Cc5ccc(-n6c(O)nnc6-c6cc(C(C)C)c(O)cc6O)cc5)CC4)N3C)nc3c2CCN(c2cccc4ccccc24)C3)C[C@@H]1CC#N]

### 2-[(2S)-4-(2-{[(2S,5R)-5-{[4-({4-[3-(2,4-dihydroxy-5-isopropylphenyl)-5-hydroxy-1,2,4-triazol-4-yl]phenyl}methyl)piperazin-1-yl]methyl}-1-methylpyrrolidin-2-yl]methoxy}-7-(naphthalen-1-yl)-5H,6H,8H-pyrido[3,4-d]pyrimidin-4-yl)-1-(prop-2-enoyl)piperazin-2-yl]acetonitrile

¹H NMR (DMSO-d₆, 400 MHz): δ 11.91 (s, 1H), 9.58 (s, 1H), 9.40 (s, 1H), 8.21-8.15 (m, 1H), 7.96-7.89 (m, 1H), 7.64 (d, J=8.2 Hz, 1H), 7.57-7.50 (m, 2H), 7.46 (t, J=7.8 Hz, 1H), 7.27 (d, J=8.1 Hz, 2H), 7.22 (d, J=7.5 Hz, 1H), 7.12 (d, J=8.3 Hz, 2H), 6.87 (s, 1H), 6.75 (s, 1H), 6.26 (s, 1H), 6.23-6.14 (m, 1H), 5.78 (d, J=11.6 Hz, 1H), 5.13-4.64 (m, 1H), 4.49-3.95 (m, 7H), 3.41 (s, 3H), 3.18 (s, 4H), 3.14-2.81 (m, 6H), 2.67 (s, 1H), 2.45-2.10 (m, 13H), 1.84 (s, 2H), 1.63-1.42 (m, 2H), 0.93 (d, J=6.9 Hz, 6H). LC-MS: m/z 973.6 [M+H]⁺.

### Compound 273

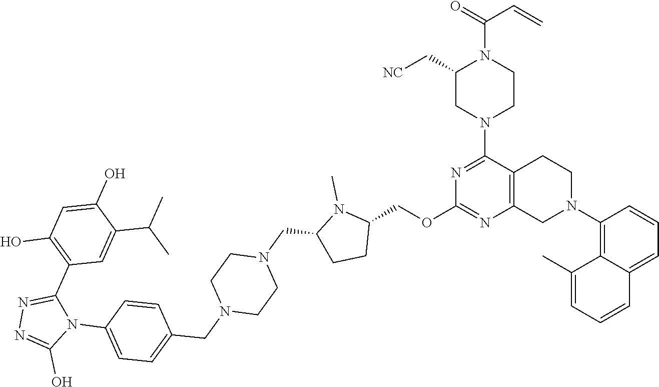 [SMILES: C=CC(=O)N1CCN(c2nc(OC[C@@H]3CC[C@H](CN4CCN(Cc5ccc(-n6c(O)nnc6-c6cc(C(C)C)c(O)cc6O)cc5)CC4)N3C)nc3c2CCN(c2cccc4cccc(C)c24)C3)C[C@@H]1CC#N]

### 2-[(2S)-4-(2-{[(2S,5R)-5-{[4-({4-[3-(2,4-dihydroxy-5-isopropylphenyl)-5-hydroxy-1,2,4-triazol-4-yl]phenyl}methyl)piperazin-1-yl]methyl}-1-methylpyrrolidin-2-yl]methoxy}-7-(8-methylnaphthalen-1-yl)-5H,6H,8H-pyrido[3,4-d]pyrimidin-4-yl)-1-(prop-2-enoyl)piperazin-2-yl]acetonitrile

¹H NMR (DMSO-d₆, 400 MHz): δ 11.91 (s, 1H), 9.58 (s, 1H), 9.40 (s, 1H), 7.75 (d, J=8.1 Hz, 1H), 7.72-7.61 (m, 1H), 7.45 (q, J=8.0 Hz, 1H), 7.40-7.30 (m, 2H), 7.27 (d, J=7.4 Hz, 3H), 7.15-7.00 (m, 2H), 6.85 (s, 1H), 6.75 (s, 1H), 6.26 (s, 1H), 6.22-6.15 (m, 1H), 5.81-5.69 (m, 1H), 5.01-4.71 (m, 1H), 4.52-4.15 (m, 2H), 4.11-3.79 (m, 5H), 3.78-3.45 (m, 2H), 3.41 (s, 4H), 3.16-2.98 (m, 5H), 2.97-2.88 (m, 2H), 2.86 (s, 3H), 2.76-2.60 (m, 2H), 2.58-2.50 (m, 2H), 2.40-2.30 (m, 9H), 2.20-2.09 (m, 1H), 1.82 (s, 2H), 1.51 (s, 2H), 1.04-0.85 (m, 6H). LC-MS: m/z 987.4 [M+H]⁺.

### Compound 274

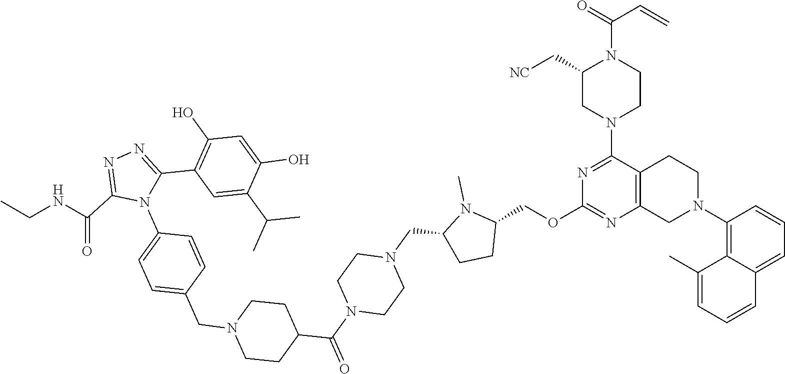 [SMILES: C=CC(=O)N1CCN(c2nc(OC[C@@H]3CC[C@H](CN4CCN(C(=O)C5CCN(Cc6ccc(-n7c(C(=O)NCC)nnc7-c7cc(C(C)C)c(O)cc7O)cc6)CC5)CC4)N3C)nc3c2CCN(c2cccc4cccc(C)c24)C3)C[C@@H]1CC#N]

### 4-(4-{[4-(4-{[(2R,5S)-5-[({4-[(3S)-3-(cyanomethyl)-4-(prop-2-enoyl)piperazin-1-yl]-7-(8-methylnaphthalen-1-yl)-5H,6H,8H-pyrido[3,4-d]pyrimidin-2-yl}oxy)methyl]-1-methylpyrrolidin-2-yl]methyl}piperazine-1-carbonyl)piperidin-1-yl]methyl}phenyl)-5-(2,4-dihydroxy-5-isopropylphenyl)-N-ethyl-1,2,4-triazole-3-carboxamide

¹H NMR (DMSO-d₆, 400 MHz): δ 10.62 (s, 1H), 9.75 (s, 1H), 8.95 (t, J=5.9 Hz, 1H), 7.88-7.66 (m, 2H), 7.45 (q, J=8.0 Hz, 1H), 7.40-7.14 (m, 7H), 6.85 (s, 1H), 6.57 (s, 1H), 6.34 (s, 1H), 6.21-6.15 (m, 1H), 5.85-5.71 (m, 1H), 5.03-4.71 (m, 1H), 4.58-4.10 (m, 2H), 4.08-3.75 (m, 5H), 3.74-3.61 (m, 1H), 3.52-3.39 (m, 7H), 3.25-2.99 (m, 7H), 2.98-2.78 (m, 7H), 2.76-2.65 (m, 2H), 2.51 (d, J=1.8 Hz, 2H), 2.43-2.24 (m, 8H), 2.21-2.06 (m, 1H), 1.99 (s, 2H), 1.84 (s, 2H), 1.56 (s, 6H), 1.03 (t, J=7.2 Hz, 3H), 0.80 (d, J=6.9 Hz, 6H). LC-MS: m/z 1153.3 [M+H]⁺.

### Compound 275

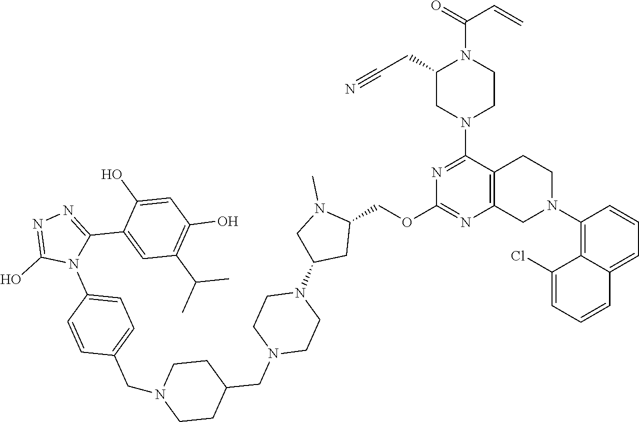 [SMILES: C=CC(=O)N1CCN(c2nc(OC[C@@H]3C[C@H](N4CCN(CC5CCN(Cc6ccc(-n7c(O)nnc7-c7cc(C(C)C)c(O)cc7O)cc6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-(4-{[1-({4-[3-(2,4-dihydroxy-5-isopropylphenyl)-5-hydroxy-1,2,4-triazol-4-yl]phenyl}methyl)piperidin-4-yl]methyl}piperazin-1-yl)-1-methylpyrrolidin-2-yl]methoxy}-5H,6H,8H-pyrido[3,4-d]pyrimidin-4-yl]-1-(prop-2-enoyl)piperazin-2-yl]acetonitrile. H NMR (DMSO-d₆, 400 MHz): δ 11.91 (s, 1H), 9.58 (s, 1H), 9.40 (s, 1H), 7.92 (dd, J=8.2, 1.3 Hz, 1H), 7.78-7.70 (m, 1H), 7.61-7.49 (m, 2H), 7.44 (t, J=7.8 Hz, 1H), 7.34 (dd, J=16.6, 7.5 Hz, 1H), 7.27 (d, J=8.1 Hz, 2H), 7.11 (d, J=8.3 Hz, 2H), 6.84 (s, 1H), 6.75 (s, 1H), 6.26 (s, 1H), 6.24-6.11 (m, 1H), 5.77 (dd, J=10.4, 2.3 Hz, 1H), 4.96-4.77 (m, 1H), 4.58-4.13 (m, 4H), 4.09-3.93 (m, 2H), 3.90-3.61 (m, 2H), 3.53-3.41 (m, 1H), 3.39 (s, 3H), 3.22-3.04 (m, 4H), 2.97 (q, J=6.7 Hz, 3H), 2.93-2.71 (m, 5H), 2.63-2.55 (m, 2H), 2.42-2.32 (m, 4H), 2.31-2.21 (m, 6H), 2.05 (d, J=7.2 Hz, 3H), 1.94-1.86 (m, 2H), 1.69-1.59 (m, 2H), 1.42 (s, 2H), 1.15-1.01 (m, 2H), 0.93 (d, J=6.9 Hz, 6H). LC-MS: m/z 1090.2 [M+H]⁺

### Compound 276

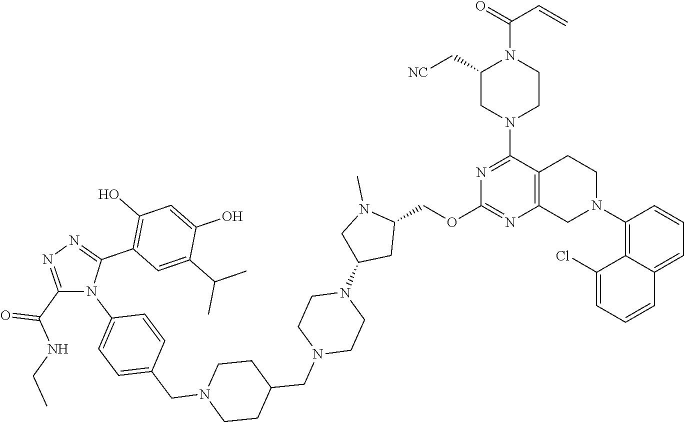 [SMILES: C=CC(=O)N1CCN(c2nc(OC[C@@H]3C[C@H](N4CCN(CC5CCN(Cc6ccc(-n7c(C(=O)NCC)nnc7-c7cc(C(C)C)c(O)cc7O)cc6)CC5)CC4)CN3C)nc3c2CCN(c2cccc4cccc(Cl)c24)C3)C[C@@H]1CC#N]

### 4-(4-{[4-({4-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]piperazin-1-yl}methyl)piperidin-1-yl]methyl}phenyl)-5-(2,4-dihydroxy-5-isopropylphenyl)-N-ethyl-1,2,4-triazole-3-carboxamide

¹H NMR (DMSO-d₆, 400 MHz): δ 10.62 (s, 1H), 9.75 (s, 1H), 8.94 (s, 1H), 7.92 (dd, J=8.2, 1.3 Hz, 1H), 7.74 (ddd, J=8.4, 3.9, 1.1 Hz, 1H), 7.59-7.50 (m, 2H), 7.45 (t, J=7.8 Hz, 1H), 7.35 (dd, J=9.0, 6.8 Hz, 3H), 7.31-7.27 (m, 2H), 6.84 (s, 1H), 6.57 (s, 1H), 6.34 (s, 1H), 6.24-6.15 (m, 1H), 5.79 (s, 1H), 4.96-4.77 (m, 1H), 4.51-4.13 (m, 4H), 4.10-3.84 (m, 3H), 3.83-3.59 (m, 2H), 3.46 (s, 4H), 3.24-3.15 (m, 3H), 3.14-3.02 (m, 3H), 3.01-2.93 (m, 2H), 2.91-2.83 (m, 3H), 2.81-2.75 (m, 2H), 2.74-2.69 (m, 1H), 2.33 (p, J=1.9 Hz, 2H), 2.27 (d, J=3.2 Hz, 9H), 2.06 (t, J=9.8 Hz, 3H), 1.98-1.86 (m, 2H), 1.69-1.61 (m, 2H), 1.57-1.37 (m, 2H), 1.03 (t, J=7.2 Hz, 5H), 0.80 (d, J=6.9 Hz, 6H). LC-MS: m/z 1145.2 [M+H]⁺.

### Compound 277

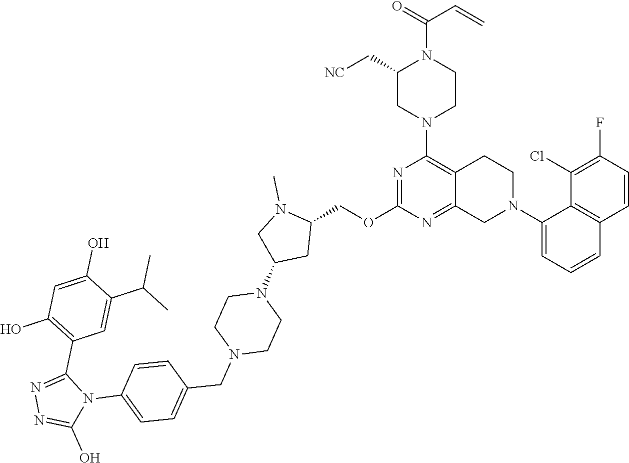 [SMILES: C=CC(=O)N1CCN(c2nc(OC[C@@H]3C[C@H](N4CCN(Cc5ccc(-n6c(O)nnc6-c6cc(C(C)C)c(O)cc6O)cc5)CC4)CN3C)nc3c2CCN(c2cccc4ccc(F)c(Cl)c24)C3)C[C@@H]1CC#N]

### 2-[(2S)-4-[7-(8-chloro-7-fluoronaphthalen-1-yl)-2-{[(2S,4S)-4-[4-({4-[3-(2,4-dihydroxy-5-isopropylphenyl)-5-hydroxy-1,2,4-triazol-4-yl]phenyl}methyl)piperazin-1-yl]-1-methylpyrrolidin-2-yl]methoxy}-5H,6H,8H-pyrido[3,4-d]pyrimidin-4-yl]-1-(prop-2-enoyl)piperazin-2-yl]acetonitrile. ¹H NMR (DMSO-d₆, 400 MHz): δ 11.91 (s, 1H), 9.57 (s, 1H), 9.39 (s, 1H), 8.01 (dd, J=9.0, 5.9 Hz, 1H), 7.79 (d, J=8.7 Hz, 1H), 7.61-7.56 (m, 1H), 7.53 (q, J=7.9 Hz, 1H), 7.45-7.38 (m, 1H), 7.27 (dd, J=8.2, 3.8 Hz, 2H), 7.11 (d, J=7.8 Hz, 2H), 6.83 (s, 1H), 6.76 (s, 1H), 6.26 (s, 1H), 6.18 (d, J=16.6 Hz, 1H), 5.77 (d, J=11.0 Hz, 1H), 4.95-4.75 (m, 1H), 4.26-3.76 (m, 11H), 3.40 (s, 5H), 3.09-2.83 (m, 8H), 2.34-2.25 (m, 10H), 2.03 (s, 1H), 1.52 (s, 1H), 0.93 (d, J=6.9 Hz, 6H). LC-MS: m/z 1011.1 [M+H]⁺

### Compound 278

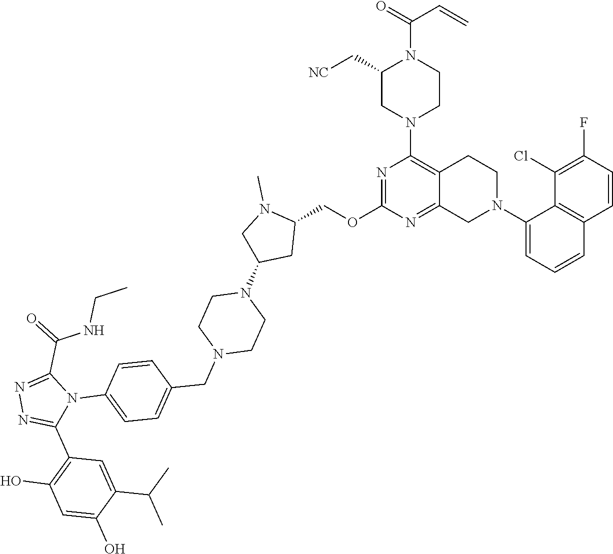 [SMILES: C=CC(=O)N1CCN(c2nc(OC[C@@H]3C[C@H](N4CCN(Cc5ccc(-n6c(C(=O)NCC)nnc6-c6cc(C(C)C)c(O)cc6O)cc5)CC4)CN3C)nc3c2CCN(c2cccc4ccc(F)c(Cl)c24)C3)C[C@@H]1CC#N]

### 4-[4-({4-[(3S,5S)-5-({[7-(8-chloro-7-fluoronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy} methyl)-1-methylpyrrolidin-3-yl]piperazin-1-yl}methyl)phenyl]-5-(2,4-dihydroxy-5-isopropylphenyl)-N-ethyl-1,2,4-triazole-3-carboxamide

¹H NMR 10.59 (s, 1H), 9.74 (s, 1H), 8.95 (t, J=5.9 Hz, 1H), 8.01 (dd, J=9.1, 5.8 Hz, 1H), 7.78 (dd, J=8.1, 3.1 Hz, 1H), 7.60 (dd, J=8.9, 1.7 Hz, 1H), 7.55-7.50 (m, 1H), 7.44 (d, J=7.5 Hz, 1H), 7.35 (dd, J=8.5, 3.1 Hz, 2H), 7.29-7.26 (m, 2H), 6.84 (s, 1H), 6.57 (s, 1H), 6.34 (s, 1H), 6.18 (d, J=16.6 Hz, 1H), 5.77 (d, J=10.5 Hz, 1H), 4.96-4.75 (m, 1H), 4.40-3.72 (m, 8H), 3.62 (s, 1H), 3.46 (s, 4H), 3.19-3.12 (m, 4H), 3.10-3.00 (m, 5H), 2.92-2.84 (m, 3H), 2.67-2.66 (m, 1H), 2.38-2.28 (m, 10H), 2.05 (s, 1H), 1.54 (s, 1H), 1.03 (t, J=7.2 Hz, 3H), 0.81-0.78 (m, 6H). LC-MS: m/z 1066.2 [M+H]⁺.

### Compound 279

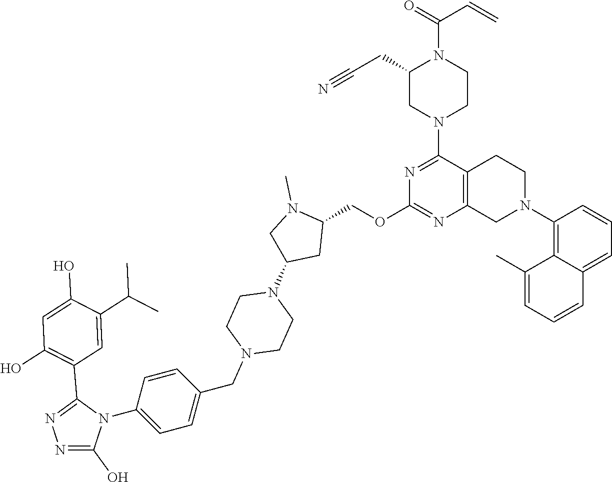 [SMILES: C=CC(=O)N1CCN(c2nc(OC[C@@H]3C[C@H](N4CCN(Cc5ccc(-n6c(O)nnc6-c6cc(C(C)C)c(O)cc6O)cc5)CC4)CN3C)nc3c2CCN(c2cccc4cccc(C)c24)C3)C[C@@H]1CC#N]

### 2-[(2S)-4-(2-{[(2S,4S)-4-[4-({4-[3-(2,4-dihydroxy-5-isopropylphenyl)-5-hydroxy-1,2,4-triazol-4-yl]phenyl}methyl)piperazin-1-yl]-1-methylpyrrolidin-2-yl]methox-y}-7-(8-methylnaphthalen-1-yl)-5H,6H,8H-pyrido[3,4-d]pyrimidin-4-yl)-1-(prop-2-enoyl)piperazin-2-yl]acetonitrile

¹H NMR (DMSO-d₆, 400 MHz): δ 11.81 (s, 1H), 9.45 (s, 1H), 9.33 (s, 1H), 7.73 (d, J=8.1 Hz, 1H), 7.68-7.61 (m, 1H), 7.44 (q, J=8.0 Hz, 1H), 7.39-7.21 (m, 5H), 7.18-7.08 (m, 2H), 6.91-6.82 (m, 1H), 6.72 (s, 1H), 6.27 (s, 1H), 6.19-6.11 (m, 1H), 5.86-5.69 (m, 1H), 4.87 (s, 1H), 4.33-4.24 (m, 1H), 4.18-4.11 (m, 1H), 4.09-3.93 (m, 3H), 3.92-3.53 (m, 2H), 3.40 (d, J=3.5 Hz, 3H), 3.18-2.89 (m, 8H), 2.88-2.81 (m, 5H), 2.80-2.65 (m, 1H), 2.58-2.48 (m, 1H), 2.43-2.29 (m, 9H), 2.27 (d, J=4.4 Hz, 4H), 2.09-2.01 (m, 1H), 1.65-1.51 (m, 1H), 0.91 (d, J=6.9 Hz, 6H). LC-MS: m/z 973.2 [M+H]⁺.

### Compound 280

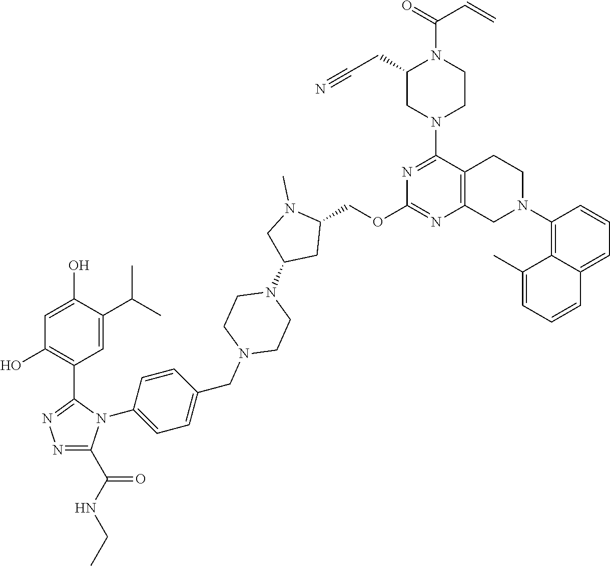 [SMILES: C=CC(=O)N1CCN(c2nc(OC[C@@H]3C[C@H](N4CCN(Cc5ccc(-n6c(C(=O)NCC)nnc6-c6cc(C(C)C)c(O)cc6O)cc5)CC4)CN3C)nc3c2CCN(c2cccc4cccc(C)c24)C3)C[C@@H]1CC#N]

### 4-[4-({4-[(3S,5S)-5-[({4-[(3S)-3-(cyanomethyl)-4-(prop-2-enoyl)piperazin-1-yl]-7-(8-methylnaphthalen-1-yl)-5H,6H,8H-pyrido[3,4-d]pyrimidin-2-yl}oxy)methyl]-1-methylpyrrolidin-3-yl]piperazin-1-yl}methyl)phenyl]-5-(2,4-dihydroxy-5-iso-propylphenyl)-N-ethyl-1,2,4-triazole-3-carboxamide

¹H NMR (DMSO-d₆, 400 MHz): δ 10.60 (s, 1H), 9.62 (s, 1H), 8.79 (t, J=5.9 Hz, 1H), 7.73 (d, J=8.1 Hz, 1H), 7.69-7.61 (m, 1H), 7.43 (q, J=7.7 Hz, 1H), 7.40-7.20 (m, 7H), 6.92-6.68 (m, 1H), 6.55 (s, 1H), 6.35 (s, 1H), 6.16-6.08 (m, 1H), 5.80-5.70 (m, 1H), 4.86 (s, 1H), 4.28 (t, J=6.0 Hz, 1H), 4.16 (m, 1H), 4.10-3.83 (m, 3H), 3.81-3.70 (m, 1H), 3.53-3.36 (m, 3H), 3.14-2.94 (m, 10H), 2.93-2.80 (m, 6H), 2.78-2.64 (m, 1H), 2.52-2.42 (m, 2H), 2.39 (s, 7H), 2.28 (d, J=4.5 Hz, 4H), 2.05 (m, 1H), 1.55 (s, 13H), 1.04-0.98 (m, 3H), 0.85-0.70 (m, 6H). LC-MS: m/z 1028.4 [M+H]⁺.

### Compound 281

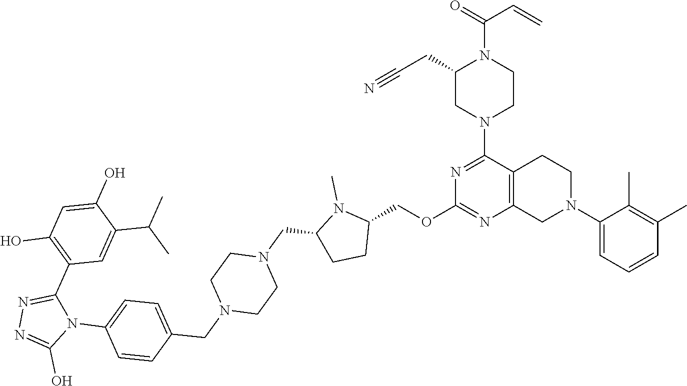 [SMILES: C=CC(=O)N1CCN(c2nc(OC[C@@H]3CC[C@H](CN4CCN(Cc5ccc(-n6c(O)nnc6-c6cc(C(C)C)c(O)cc6O)cc5)CC4)N3C)nc3c2CCN(c2cccc(C)c2C)C3)C[C@@H]1CC#N]

### 2-[(2S)-4-(2-{[(2S,5R)-5-{[4-({4-[3-(2,4-dihydroxy-5-isopropylphenyl)-5-hydroxy-1,2,4-triazol-4-yl]phenyl}methyl)piperazin-1-yl]methyl}-1-methylpyrrolidin-2-yl]methoxy}-7-(2,3-dimethylphenyl)-5H,6H,8H-pyrido[3,4-d]pyrimidin-4-yl)-1-(prop-2-enoyl)piperazin-2-yl]acetonitrile

¹H NMR (DMSO-d₆, 400 MHz): δ 11.91 (s, 1H), 9.58 (s, 1H), 9.40 (s, 1H), 7.28 (d, J=8.4 Hz, 2H), 7.12-7.07 (m, 3H), 6.97 (d, J=7.9 Hz, 4H), 6.26 (s, 1H), 6.18 (dd, J=16.7, 2.3 Hz, 1H), 5.77 (dd, J=10.4, 2.3 Hz, 1H), 4.97-4.77 (m, 1H), 4.22 (d, J=6.9 Hz, 5H), 3.96 (d, J=13.6 Hz, 2H), 3.88 (s, 4H), 3.10-3.07 (m, 3H), 2.95 (dd, J=11.7, 6.0 Hz, 4H), 2.78 (s, 3H), 2.66 (s, 1H), 2.36 (d, J=13.8 Hz, 10H), 2.22 (d, J=11.2 Hz, 8H), 1.83 (s, 2H), 1.46 (s, 2H), 0.93 (d, J=6.9 Hz, 6H). LC-MS: m/z 951.4 [M+H]⁺.

### Compound 282

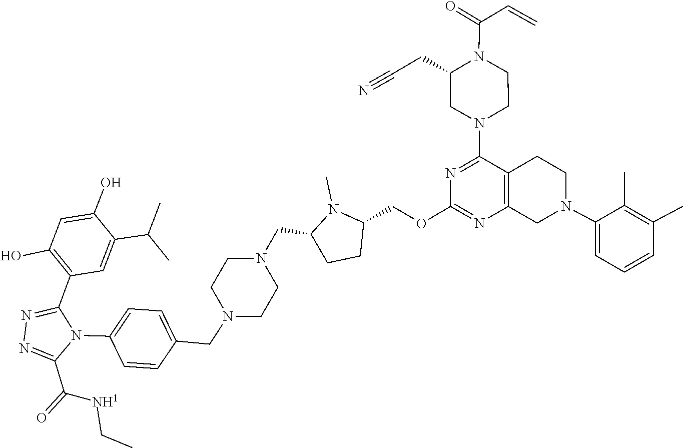

### 4-{4-[(4-{[(2R,5S)-5-[({4-[(3S)-3-(cyanomethyl)-4-(prop-2-enoyl)piperazin-1-yl]-7-(2,3-dimethylphenyl)-5H,6H,8H-pyrido[3,4-d]pyrimidin-2-yl}oxy)methyl]-1-methylpyrrolidin-2-yl]methyl}piperazin-1-yl)methyl]phenyl}-5-(2,4-dihydroxy-5-iso propylphenyl)-N-ethyl-1,2,4-triazole-3-carboxamide

¹H NMR (DMSO-d₆, 400 MHz): δ 10.59 (s, 1H), 9.74 (s, 1H), 8.94 (t, J=5.9 Hz, 1H), 7.36 (t, J=8.1 Hz, 2H), 7.29 (t, J=8.2 Hz, 2H), 7.07 (t, J=7.7 Hz, 1H), 6.97 (d, J=7.9 Hz, 1H), 6.92 (t, J=7.4 Hz, 1H), 6.83 (s, 1H), 6.57 (s, 1H), 6.34 (s, 1H), 6.18 (t, J=6.5 Hz, 1H), 5.77 (t, J=3.9 Hz, 1H), 4.97-4.77 (m, 1H), 4.75 (s, 1H), 4.38 (s, 1H), 4.24 (s, 1H), 4.22 (t, J=7.0 Hz, 6H), 3.47 (t, J=6.8 Hz, 2H), 3.31 (s, 1H), 3.15 (q, J=6.8 Hz, 5H), 2.92 (m, 3H), 2.89 (s, 2H), 2.87 (s, 2H), 2.79 (s, 9H), 2.39 (t, J=3.6 Hz, 8H), 1.84 (s, 2H), 1.51 (s, 2H), 1.03 (t, J=7.2 Hz, 3H), 0.80 (t, J=6.9 Hz, 6H). LC-MS: m/z 1006.2 [M+H]⁺.

### Compound 283

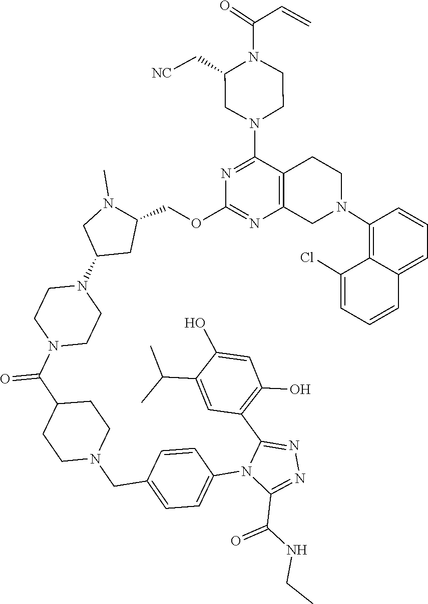 [SMILES: C=CC(=O)N1CCN(c2nc(OC[C@@H]3C[C@H](N4CCN(C(=O)C5CCN(Cc6ccc(-n7c(C(=O)NCC)nnc7-c7cc(C(C)C)c(O)cc7O)cc6)CC5)CC4)CN3C)nc3c2CCN(c2cccc4cccc(Cl)c24)C3)C[C@@H]1CC#N]

### 4-{4-[(4-{4-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]piperazine-1-carbonyl}piperidin-1-yl)methyl]phenyl}-5-(2,4-dihydroxy-5-isopropylphenyl)-N-ethyl-1,2,4-triazole-3-carboxamide

¹H NMR (DMSO-d₆, 400 MHz): δ 10.61 (s, 1H), 9.74 (s, 1H), 8.95 (t, J=5.8 Hz, 1H), 7.92 (d, J=8.1 Hz, 1H), 7.74 (dd, J=8.0, 4.3 Hz, 1H), 7.62-7.41 (m, 2H), 7.40-7.26 (m, 6H), 6.85 (s, 1H), 6.57 (s, 1H), 6.34 (s, 1H), 6.18 (d, J=16.6 Hz, 1H), 5.77 (d, J=11.1 Hz, 1H), 4.96 (s, 1H), 4.76 (s, 6H), 4.28 (s, 2H), 4.18 (t, J=15.3 Hz, 8H), 3.16 (dd, J=7.4, 6.0 Hz, 2H), 3.03-2.94 (m, 9H), 2.93-2.88 (m, 2H), 2.86-2.78 (m, 9H), 2.37 (s, 4H), 2.30-2.26 (m, 5H), 1.03 (t, J=7.2 Hz, 3H), 0.80 (d, J=6.8 Hz, 6H). LC-MS: m/z 1159.2 [M+H]⁺.

### Compound 284

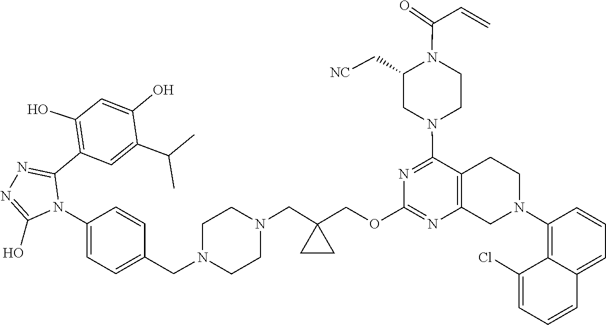 [SMILES: C=CC(=O)N1CCN(c2nc(OCC3(CN4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N]

### 2-[(2S)-4-[7-(8-chloronaphthalen-1-yl)-2-[(1-{[4-({4-[3-(2,4-dihydroxy-5-isopropyl phenyl)-5-hydroxy-1,2,4-triazol-4-yl]phenyl}methyl)piperazin-1-yl]methyl}cyclo propyl)methoxy]-5H,6H,8H-pyrido[3,4-d]pyrimidin-4-yl]-1-(prop-2-enoyl)pipera zin-2-yl]acetonitrile

¹H NMR (DMSO-d₆, 400 MHz): δ 11.93 (s, 1H), 9.57 (s, 1H), 9.40 (s, 1H), 7.93 (d, J=8.4 Hz, 1H), 7.84 (J=9.6 Hz, 1H), 7.73-7.72 (m, 2H), 7.58-7.56 (m, 1H), 7.53-7.52 (m, 1H), 7.46-7.44 (m, 2H), 7.36-7.10 (m, 2H), 6.74 (br, 1H), 6.69 (s, 1H), 6.26 (s, 1H), 6.17 (d, J=8.4 Hz, 1H), 5.77 (d, J=12.0 Hz, 1H), 4.96-4.68 (m, 1H), 4.11-3.67 (m, 8H), 3.60-3.55 (m, 1H), 3.50-3.48 (m, 2H), 2.29-2.11 (m, 2H), 3.07-2.98 (m, 2H), 2.45-2.25 (m, 6H), 2.06-1.97 (m, 1H), 1.23-1.09 (m, 6H), 0.91-0.84 (m, 6H), 0.55 (s, 2H), 0.36 (s, 2H). LC-MS: m/z 964.2 [M+H]⁺.

### Compound 285

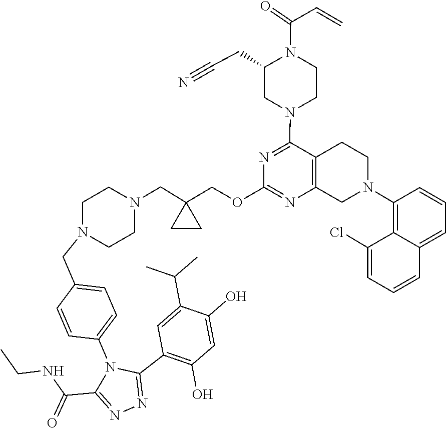 [SMILES: C=CC(=O)N1CCN(c2nc(OCC3(CN4CCN(Cc5ccc(-n6c(C(=O)NCC)nnc6-c6cc(C(C)C)c(O)cc6O)cc5)CC4)CC3)nc3c2CCN(c2cccc4cccc(Cl)c24)C3)C[C@@H]1CC#N]

### 4-{4-[(4-{[1-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)cyclopropyl]methyl}piperazin-1-yl)methyl]phenyl}-5-(2,4-dihydroxy-5-isopropylphenyl)-N-ethyl-1,2,4-triazole-3-carboxamide

¹H NMR (DMSO-d₆, 400 MHz): δ 10.70 (s, 1H), 9.83 (s, 1H), 9.00 (t, J=5.9 Hz, 1H), 7.98 (d, J=8.2 Hz, 1H), 7.81 (dd, J=8.6, 3.6 Hz, 1H), 7.62 (dd, J=18.3, 7.7 Hz, 2H), 7.55-7.45 (m, 1H), 7.44-7.30 (m, 5H), 6.91 (s, 1H), 6.63 (s, 1H), 6.41 (s, 1H), 6.25 (d, J=16.6 Hz, 1H), 5.84 (d, J=10.6 Hz, 1H), 4.93-4.74 (m, 1H), 4.23-3.46 (m, 9H), 3.43-2.83 (m, 9H), 2.55-2.45 (m, 8H), 1.30 (s, 3H), 1.10 (t, J=7.2 Hz, 3H), 0.85 (d, J=6.9 Hz, 6H), 0.64 (s, 2H), 0.44 (s, 2H). LC-MS: m/z 1019.3 [M+H]⁺.

### Compound 286

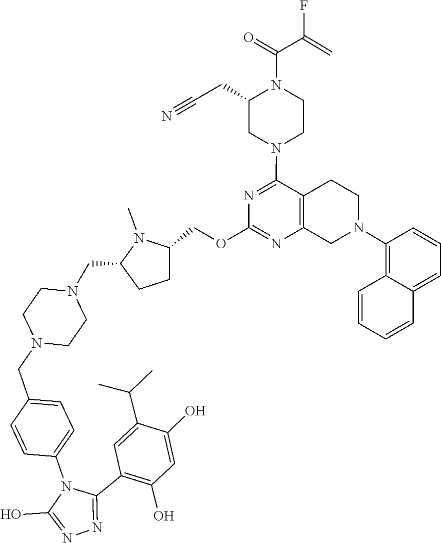 [SMILES: C=C(F)C(=O)N1CCN(c2nc(OC[C@@H]3CC[C@H](CN4CCN(Cc5ccc(-n6c(O)nnc6-c6cc(C(C)C)c(O)cc6O)cc5)CC4)N3C)nc3c2CCN(c2cccc4ccccc24)C3)C[C@@H]1CC#N]

### 2-[(2S)-4-(2-{[(2S,5R)-5-{[4-({4-[3-(2,4-dihydroxy-5-isopropylphenyl)-5-hydroxy-1,2,4-triazol-4-yl]phenyl}methyl) piperazin-1-yl]methyl}-1-methylpyrrolidin-2-yl]methoxy}-7-(naphthalen-1-yl)-5H,6H,8H-pyrido[3,4-d]pyrimidin-4-yl)-1-(2-fluoroprop-2-enoyl) piperazin-2-yl]acetonitrile

¹H NMR (DMSO-d₆, 400 MHz): δ 11.91 (s, 1H), 9.57 (s, 1H), 9.40 (s, 1H), 8.18 (dt, J=6.4, 3.5 Hz, 1H), 7.92 (dt, J=6.9, 3.5 Hz, 1H), 7.64 (d, J=8.2 Hz, 1H), 7.54 (dt, J=6.3, 3.4 Hz, 2H), 7.46 (t, J=7.8 Hz, 1H), 7.28 (d, J=8.2 Hz, 2H), 7.22 (d, J=7.4 Hz, 1H), 7.15-7.09 (m, 2H), 6.75 (s, 1H), 6.26 (s, 1H), 5.45-5.37 (m, 1H), 5.36-5.19 (m, 1H), 5.05-4.18 (m, 3H), 4.14 (s, 2H), 4.04 (d, J=12.8 Hz, 4H), 3.83-3.52 (m, 1H), 3.42 (s, 3H), 3.26-3.14 (m, 3H), 3.09-2.85 (m, 5H), 2.68 (s, 1H), 2.37 (s, 12H), 2.22-2.14 (m, 1H), 1.84 (s, 2H), 1.61-1.38 (m, 2H), 0.93 (d, J=6.9 Hz, 6H). LC-MS: m/z 991.7 [M+H]⁺.

### Compound 287

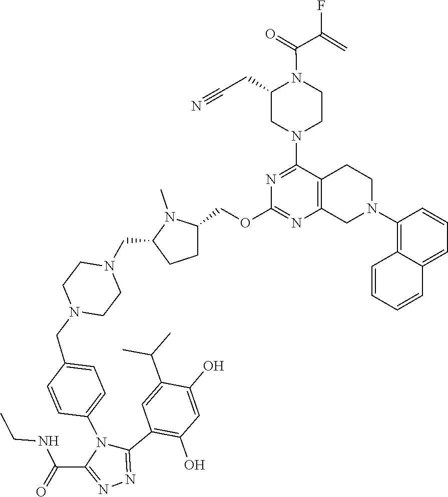 [SMILES: C=C(F)C(=O)N1CCN(c2nc(OC[C@@H]3CC[C@H](CN4CCN(Cc5ccc(-n6c(C(=O)NCC)nnc6-c6cc(C(C)C)c(O)cc6O)cc5)CC4)N3C)nc3c2CCN(c2cccc4ccccc24)C3)C[C@@H]1CC#N]

### 4-{4-[(4-{[(2R,5S)-5-[({4-[(3S)-3-(cyanomethyl)-4-(2-fluoroprop-2-enoyl)piperazin-1-yl]-7-(naphthalen-1-yl)-5H,6H,8H-pyrido[3,4-d]pyrimidin-2-yl}oxy)methyl]-1-methylpyrrolidin-2-yl]methyl}piperazin-1-yl)methyl]phenyl}-5-(2,4-dihydroxy-5-isopropylphenyl)-N-ethyl-1,2,4-triazole-3-carboxamide

¹H NMR (DMSO-d₆, 400 MHz): δ 10.60 (s, 1H), 9.74 (s, 1H), 8.94 (t, J=5.9 Hz, 1H), 8.18 (dt, J=6.4, 3.5 Hz, 1H), 7.96-7.88 (m, 1H), 7.64 (d, J=8.2 Hz, 1H), 7.54 (dt, J=6.3, 3.3 Hz, 2H), 7.46 (t, J=7.8 Hz, 1H), 7.36 (d, J=8.4 Hz, 2H), 7.29 (d, J=8.4 Hz, 2H), 7.23 (d, J=7.4 Hz, 1H), 6.57 (s, 1H), 6.34 (s, 1H), 5.44-5.37 (m, 1H), 5.36-5.20 (m, 1H), 4.84 (s, 1H), 4.24 (dd, J=10.6, 4.7 Hz, 1H), 4.14 (s, 2H), 4.11-3.61 (m, 4H), 3.48 (s, 2H), 3.24-3.10 (m, 8H), 3.06-2.84 (m, 5H), 2.70 (s, 1H), 2.40 (s, 12H), 2.19 (dd, J=12.2, 6.8 Hz, 1H), 1.85 (s, 2H), 1.59-1.42 (m, 2H), 1.03 (t, J=7.2 Hz, 3H), 0.80 (d, J=6.9 Hz, 6H). LC-MS: m/z 1046.7 [M+H]⁺.

### Compound 288

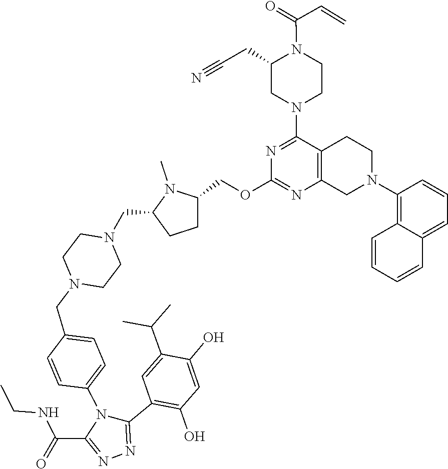 [SMILES: C=CC(=O)N1CCN(c2nc(OC[C@@H]3CC[C@H](CN4CCN(Cc5ccc(-n6c(C(=O)NCC)nnc6-c6cc(C(C)C)c(O)cc6O)cc5)CC4)N3C)nc3c2CCN(c2cccc4ccccc24)C3)C[C@@H]1CC#N]

### 4-{4-[(4-{[(2R,5S)-5-[({4-[(3S)-3-(cyanomethyl)-4-(prop-2-enoyl) piperazin-1-yl]-7-(naphthalen-1-yl)-5H,6H,8H-pyrido[3,4-d]pyrimidin-2-yl}oxy) methyl]-1-methylpyrrolidin-2-yl]methyl}piperazin-1-yl) methyl]phenyl}-5-(2,4-dihydroxy-5-isopropylphenyl)-N-ethyl-1,2,4-triazole-3-carboxamide

¹H NMR (DMSO-d₆, 400 MHz): δ 10.60 (s, 1H), 9.74 (s, 1H), 8.94 (t, J=5.9 Hz, 1H), 8.22-8.15 (m, 1H), 7.96-7.89 (m, 1H), 7.64 (d, J=8.2 Hz, 1H), 7.54 (dt, J=5.1, 3.4 Hz, 2H), 7.46 (t, J=7.8 Hz, 1H), 7.36 (d, J=8.1 Hz, 2H), 7.29 (d, J=8.3 Hz, 2H), 7.23 (d, J=7.4 Hz, 1H), 6.86 (s, 1H), 6.57 (s, 1H), 6.34 (s, 1H), 6.24-6.15 (m, 1H), 5.78 (dd, J=10.4, 2.3 Hz, 1H), 5.06-4.70 (m, 1H), 4.52-3.92 (m, 7H), 3.48 (s, 3H), 3.23-3.11 (m, 5H), 3.10-2.82 (m, 6H), 2.70 (s, 1H), 2.40 (s, 12H), 2.20 (s, 2H), 1.86 (s, 2H), 1.62-1.42 (m, 2H), 1.03 (t, J=7.2 Hz, 3H), 0.80 (d, J=6.9 Hz, 6H). LC-MS: m/z 1028.4 [M+H]⁺.

### Compound 289

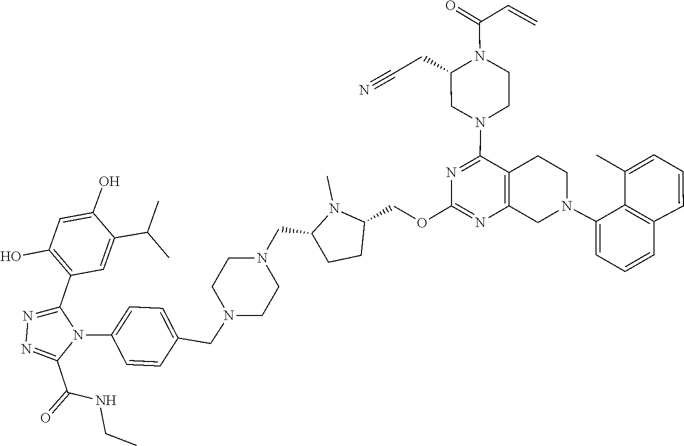 [SMILES: C=CC(=O)N1CCN(c2nc(OC[C@@H]3CC[C@H](CN4CCN(Cc5ccc(-n6c(C(=O)NCC)nnc6-c6cc(C(C)C)c(O)cc6O)cc5)CC4)N3C)nc3c2CCN(c2cccc4cccc(C)c24)C3)C[C@@H]1CC#N]

### 4-{4-[(4-{[(2R,5S)-5-[({4-[(3S)-3-(cyanomethyl)-4-(prop-2-enoyl)piperazin-1-yl]-7-(8-methylnaphthalen-1-yl)-5H,6H,8H-pyrido[3,4-d]pyrimidin-2-yl}oxy)methyl]-1-methylpyrrolidin-2-yl]methyl}piperazin-1-yl)methyl]phenyl}-5-(2,4-dihydroxy-5-isopropylphenyl)-N-ethyl-1,2,4-triazole-3-carboxamide

¹H NMR (DMSO-d₆, 400 MHz): δ 10.59 (s, 1H), 9.74 (s, 1H), 8.94 (t, J=5.9 Hz, 1H), 7.75 (d, J=8.1 Hz, 1H), 7.69 (dd, J=8.1, 5.3 Hz, 1H), 7.50-7.40 (m, 1H), 7.40-7.22 (m, 7H), 6.85 (s, 1H), 6.57 (s, 1H), 6.34 (s, 1H), 6.22-6.10 (m, 1H), 5.86-5.70 (m, 1H), 5.02-4.70 (m, 1H), 4.54-3.82 (m, 6H), 3.80-3.60 (m, 1H), 3.47 (s, 3H), 3.28-3.02 (m, 11H), 2.95-2.78 (m, 6H), 2.45-2.32 (m, 10H), 2.18 (s, 2H), 1.84 (s, 2H), 1.49 (s, 2H), 1.03 (t, J=7.2 Hz, 3H), 0.80 (d, J=6.9 Hz, 6H). LC-MS: m/z 1042.3 [M+H]⁺.

### Compound 290

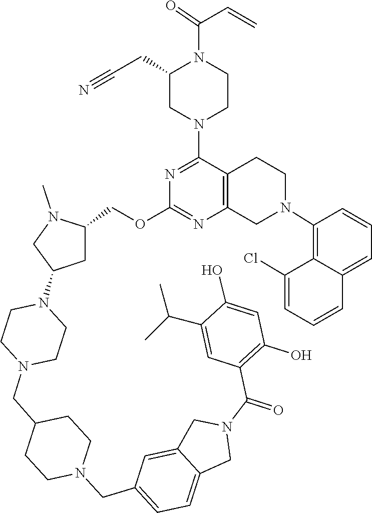 [SMILES: C=CC(=O)N1CCN(c2nc(OC[C@@H]3C[C@H](N4CCN(CC5CCN(Cc6ccc7c(c6)CN(C(=O)c6cc(C(C)C)c(O)cc6O)C7)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{4-[(1-{[2-(2,4-dihydroxy-5-isopropylbenzoyl)-1,3-dihydroisoindol-5-yl]methyl}piperidin-4-yl)methyl]piperazin-1-yl}-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 10.07 (s, 1H), 9.60 (s, 1H), 7.95-7.87 (m, 1H), 7.74 (dd, J=8.2, 4.1 Hz, 1H), 7.61-7.48 (m, 2H), 7.44 (t, J=7.8 Hz, 1H), 7.39-7.29 (m, 1H), 7.23-7.19 (m, 3H), 7.05 (s, 1H), 6.85 (s, 1H), 6.39 (s, 1H), 6.26-6.08 (m, 1H), 5.89-5.72 (m, 1H), 4.95-4.40 (m, 6H), 4.30-3.92 (m, 6H), 3.89-3.71 (m, 2H), 3.62-3.46 (m, 2H), 3.40 (s, 2H), 3.14-3.01 (m, 5H), 2.98-2.89 (m, 2H), 2.85 (s, 1H), 2.78-2.65 (m, 3H), 2.41-2.21 (m, 10H), 2.05 (d, J=7.1 Hz, 3H), 1.86 (s, 2H), 1.66-1.56 (m, 2H), 1.45 (s, 2H), 1.38 (d, J=2.8 Hz, 2H), 1.23 (s, 1H), 1.13 (d, J=6.9 Hz, 6H), 1.11-1.02 (m, 2H). LC-MS: m/z 1076.2 [M+H]⁺.

### Compound 291

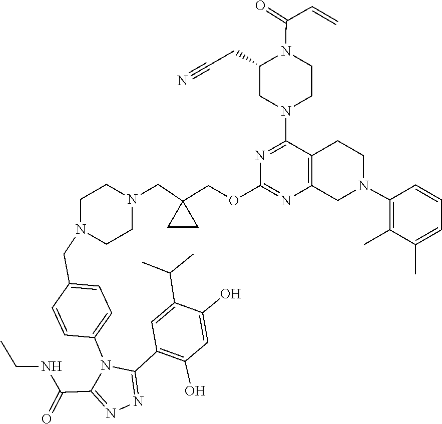 [SMILES: C=CC(=O)N1CCN(c2nc(OCC3(CN4CCN(Cc5ccc(-n6c(C(=O)NCC)nnc6-c6cc(C(C)C)c(O)cc6O)cc5)CC4)CC3)nc3c2CCN(c2cccc(C)c2C)C3)C[C@@H]1CC#N]

### 4-(4-{[4-({1-[({4-[(3S)-3-(cyanomethyl)-4-(prop-2-enoyl)piperazin-1-yl]-7-(2,3-di methylphenyl)-5H,6H,8H-pyrido[3,4-d]pyrimidin-2-yl}oxy)methyl]cyclopropyl}-methyl)piperazin-1-yl]methyl}phenyl)-5-(2,4-dihydroxy-5-isopropylphenyl)-N-et-hyl-1,2,4-triazole-3-carboxamide

¹H NMR (CDCl₃, 400 MHz): δ 10.62 (s, 1H), 9.75 (s, 1H), 8.95 (t, J=5.9 Hz, 1H), 7.35 (d, J=8.1 Hz, 2H), 7.28 (d, J=8.0 Hz, 2H), 7.07 (t, J=7.7 Hz, 1H), 6.97 (d, J=7.9 Hz, 2H), 6.94-6.74 (m, 1H), 6.56 (s, 1H), 6.34 (s, 1H), 6.18 (dd, J=16.6, 2.3 Hz, 1H), 5.77 (d, J=11.0 Hz, 1H), 5.02-4.75 (m, 1H), 4.43-4.12 (m, 3H), 4.02-3.88 (m, 5H), 3.47 (s, 3H), 3.24-3.05 (m, 6H), 3.05-2.92 (m, 3H), 2.87 (q, J=6.9 Hz, 2H), 2.79 (s, 2H), 2.36 (s, 5H), 2.28 (s, 2H), 2.23 (d, J=10.0 Hz, 6H), 1.03 (t, J=7.2 Hz, 3H), 0.79 (d, J=6.9 Hz, 6H), 0.57 (s, 2H), 0.37 (s, 2H). LC-MS: m/z 963.3 [M+H]⁺.

### Compound 292

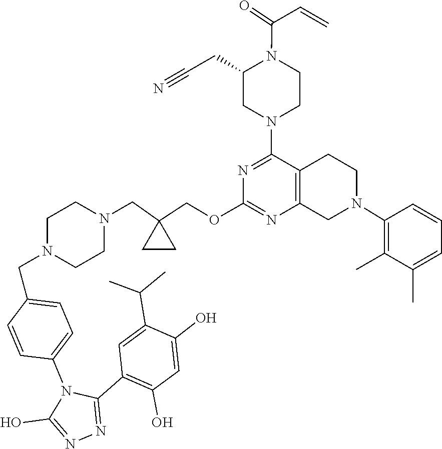 [SMILES: C=CC(=O)N1CCN(c2nc(OCC3(CN4CCN(Cc5ccc(-n6c(O)nnc6-c6cc(C(C)C)c(O)cc6O)cc5)CC4)CC3)nc3c2CCN(c2cccc(C)c2C)C3)C[C@@H]1CC#N]

### 2-[(2S)-4-{2-[(1-{[4-({4-[3-(2,4-dihydroxy-5-isopropylphenyl)-5-hydroxy-1,2,4-triazol-4-yl]phenyl}methyl)piperazin-1-yl]methyl}cyclopropyl)methoxy]-7-(2,3-dimethylphenyl)-5H,6H,8H-pyrido[3,4-d]pyrimidin-4-yl}-1-(prop-2-enoyl)piperazin-2-yl]acetonitrile

¹H NMR (DMSO-d₆, 400 MHz): δ 11.92 (s, 1H), 9.59 (s, 1H), 9.41 (s, 1H), 7.30-7.23 (m, 2H), 7.16-7.03 (m, 3H), 7.01-6.92 (m, 2H), 6.85 (s, 1H), 6.75 (s, 1H), 6.25-6.15 (m, 2H), 5.77 (dd, J=10.3, 2.2 Hz, 1H), 4.95-4.75 (m, 1H), 4.40 (s, 1H), 4.11 (s, 2H), 4.08-4.01 (m, 2H), 3.88 (s, 2H), 3.52 (s, 1H), 3.40 (s, 2H), 3.33 (s, 2H), 3.11 (t, J=15.1 Hz, 3H), 2.95-2.85 (m, 4H), 2.79 (s, 2H), 2.48-2.31 (m, 6H), 2.19-2.15 (m, 8H), 0.92 (d, J=6.9 Hz, 6H), 0.55 (d, J=4.5 Hz, 2H), 0.37 (d, J=5.1 Hz, 2H). LC-MS: m/z 908.2 [M+H]⁺.

### Compound 293

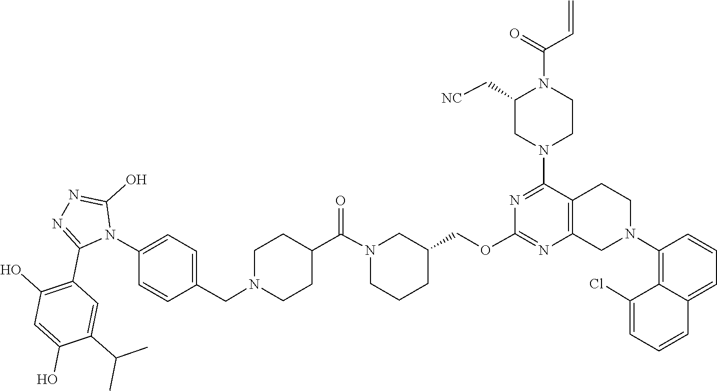 [SMILES: C=CC(=O)N1CCN(c2nc(OC[C@@H]3CCCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N]

### 2-((S)-1-acryloyl-4-(7-(8-chloronaphthalen-1-yl)-2-(((R)-1-(1-(4-(3-(2,4-dihydroxy-5-isopropylphenyl)-5-hydroxy-4H-1,2,4-triazol-4-yl)benzyl)piperidine-4-carbonyl)piperidin-3-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.92 (s, 1H), 9.59 (s, 1H), 9.42 (s, 1H), 7.91 (d, J=6.8 Hz, 2H), 7.59-7.09 (m, 8H), 6.75-6.61 (m, 2H), 6.27-6.16 (m, 2H), 5.77 (d, J=10.4 Hz, 1H), 5.06-4.81 (m, 1H), 4.22-3.29 (m, 12H), 3.14-2.67 (m, 13H), 1.98-1.23 (m, 12H), 0.94-0.91 (m, 6H). LC-MS: m/z 1220.5 [M+H]⁺.

### Compound 294

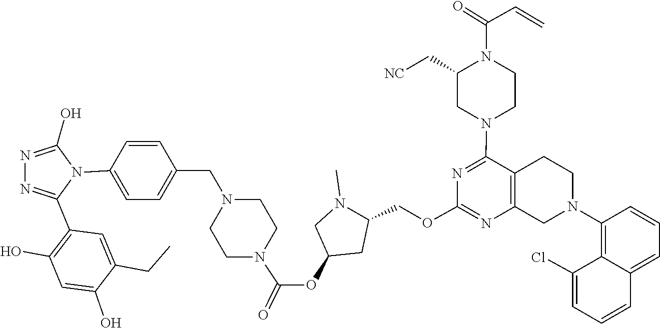 [SMILES: C=CC(=O)N1CCN(c2nc(OC[C@@H]3C[C@@H](OC(=O)N4CCN(Cc5ccc(-n6c(O)nnc6-c6cc(CC)c(O)cc6O)cc5)CC4)CN3C)nc3c2CCN(c2cccc4cccc(Cl)c24)C3)C[C@@H]1CC#N]

### (3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl 4-(4-(3-(5-ethyl-2,4-dihydroxyphenyl)-5-hydroxy-4H-1,2,4-triazol-4-yl)benzyl)piperazine-1-carboxylate

¹H NMR (CD₃OD, 400 MHz): δ 7.74 (d, J=8.0 Hz, 1H), 7.61 (d, J=8.0 Hz, 1H), 7.47-7.38 (m, 4H), 7.30-7.22 (m, 4H), 6.79 (s, 1H), 6.73-6.67 (m, 1H), 6.19 (d, J=16.4 Hz, 1H), 6.11 (s, 1H), 5.74 (d, J=10.8 Hz, 1H), 5.28 (m, 1H), 4.96-4.86 (m, 1H), 4.53-4.50 (m, 2H), 4.28-3.98 (m, 7H), 3.72-3.39 (m, 7H), 3.18-2.97 (m, 12H), 2.82-2.58 (m, 3H), 2.45-2.32 (m, 2H), 2.32 (q, J=7.6 Hz, 2H), 0.93 (t, J=7.6 Hz, 3H). LC-MS: m/z 1023.5 [M+H]⁺.

### Compound 295

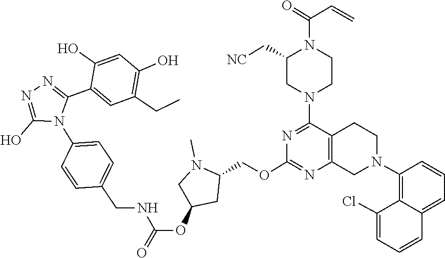 [SMILES: C=CC(=O)N1CCN(c2nc(OC[C@@H]3C[C@@H](OC(=O)NCc4ccc(-n5c(O)nnc5-c5cc(CC)c(O)cc5O)cc4)CN3C)nc3c2CCN(c2cccc4cccc(Cl)c24)C3)C[C@@H]1CC#N]

### (3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl 4-(3-(5-ethyl-2,4-dihydroxyphenyl)-5-hydroxy-4H-1,2,4-triazol-4-yl)benzylcarbamate

¹H NMR (DMSO-d₆, 400 MHz): δ 11.87 (s, 1H), 10.09 (brs, 1H), 9.54 (s, 1H), 9.33 (s, 1H), 7.93 (d, J=7.6 Hz, 1H), 7.76 (dd, J=7.6, 3.6 Hz, 1H), 7.59-7.51 (m, 2H), 7.45 (dd, J=8.0, 7.6 Hz, 1H), 7.35 (dd, J=16.0, 7.6 Hz, 1H), 7.23 (d, J=8.4 Hz, 2H), 7.12 (d, J=8.4 Hz, 1H), 6.88 (s, 1H), 6.87-6.85 (m, 1H), 6.23 (s, 1H), 6.19 (d, J=16.4 Hz, 1H), 5.79 (d, J=10.8 Hz, 1H), 5.18-5.17 (m, 1H), 4.97-4.95 (m, 1H), 4.77-4.45 (m, 3H), 4.24-4.18 (m, 4H), 3.95-3.92 (m, 2H), 3.81-3.51 (m, 4H), 3.30-3.06 (m, 6H), 3.00 (s, 3H), 2.92-2.67 (m, 2H), 2.39-2.21 (m, 4H), 1.00 (t, J=7.6 Hz, 3H). LC-MS: m/z 954.4 [M+H]⁺.

### Compound 296

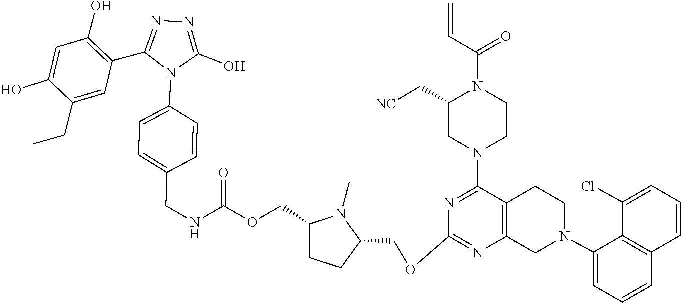 [SMILES: C=CC(=O)N1CCN(c2nc(OC[C@@H]3CC[C@H](COC(=O)NCc4ccc(-n5c(O)nnc5-c5cc(CC)c(O)cc5O)cc4)N3C)nc3c2CCN(c2cccc4cccc(Cl)c24)C3)C[C@@H]1CC#N]

### ((2R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-2-yl)methyl4-(3-(5-ethyl-2,4-dihydroxyphenyl)-5-hydroxy-4H-1,2,4-triazol-4-yl)benzylcarbamate

¹H NMR (DMSO-d₆, 400 MHz): δ 11.86 (s, 1H), 9.51 (s, 1H), 9.32 (s, 1H), 7.92 (d, 1H), 7.90-7.73 (m, 2H), 7.69-7.52 (m, 2H), 7.66-7.64 (t, 1H), 7.34-7.30 (dd, 1H), 7.21-7.19 (d, 2H), 7.11-7.09 (d, 2H) 6.86 (m, 1H), 6.22-6.19 (m, 2H), 5.78 (d, 1H), 4.98-4.73 (m, 1H), 4.41-3.92 (m, 13H), 3.29-2.54 (m, 8H) 2.39-2.32 (m, 5H), 1.97-1.53 (m, 4H), 1.01-0.97 (t, 3H). LC-MS: m/z 968.4 [M+H]+

### Compound 297

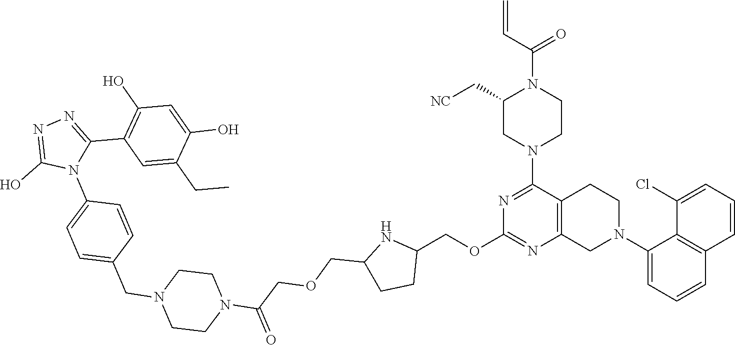 [SMILES: C=CC(=O)N1CCN(c2nc(OCC3CCC(COCC(=O)N4CCN(Cc5ccc(-n6c(O)nnc6-c6cc(CC)c(O)cc6O)cc5)CC4)N3)nc3c2CCN(c2cccc4cccc(Cl)c24)C3)C[C@@H]1CC#N]

### 2-((2S)-1-acryloyl-4-(7-(8-chloronaphthalen-1-yl)-2-((5-((2-(4-(4-(3-(5-ethyl-2,4-dihydroxyphenyl)-5-hydroxy-4H-1,2,4-triazol-4-yl)benzyl)piperazin-1-yl)-2-oxoethoxy)methyl)pyrrolidin-2-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.95 (s, 1H), 9.72 (brs, 1H), 9.58 (s, 1H), 9.32 (s, 1H), 7.92 (d, J=8.0 Hz, 1H), 7.75 (dd, J=8.0 Hz, 3.6 Hz, 1H), 7.58-7.24 (m, 8H), 6.84 (brs, 1H), 6.24 (s, 1H), 6.18 (d, J=17.2 Hz, 1H), 5.78 (d, J=10.4 Hz, 1H), 4.97-4.76 (m, 1H), 4.51-2.75 (m, 31H), 2.39-2.32 (m, 3H), 2.14-2.08 (m, 2H), 1.78 (brs, 2H), 0.99 (t, J=7.2 Hz, 3H). LC-MS: m/z 1037.5 [M+H]⁺.

### Compound 298

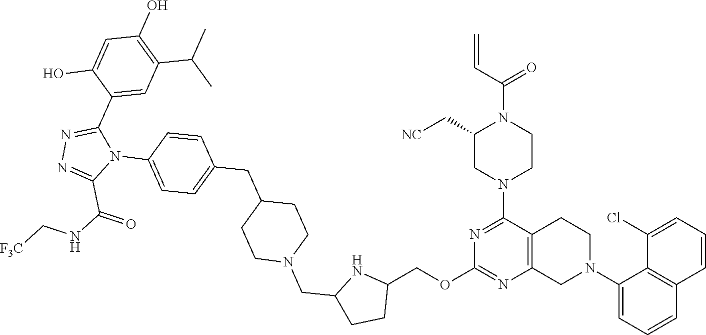 [SMILES: C=CC(=O)N1CCN(c2nc(OCC3CCC(CN4CCC(Cc5ccc(-n6c(C(=O)NCC(F)(F)F)nnc6-c6cc(C(C)C)c(O)cc6O)cc5)CC4)N3)nc3c2CCN(c2cccc4cccc(Cl)c24)C3)C[C@@H]1CC#N]

### 4-(4-((1-((5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)pyrrolidin-2-yl)methyl)piperidin-4-yl)methyl)phenyl)-5-(2,4-dihydroxy-5-isopropylphenyl)-N-(2,2,2-trifluoroethyl)-4H-1,2,4-triazole-3-carboxamide

¹H NMR (DMSO-d₆, 400 MHz): δ 9.78 (s, 1H), 9.60 (t, J=6.0 Hz, 1H). 9.39 (brs, 1H), 9.24 (brs, 1H), 8.81 (brs, 1H), 7.92 (d, J=7.6 Hz, 1H), 7.75 (dd, J=8.0 Hz, 4.0 Hz, 1H), 7.58 (d, J=7.6 Hz, 1H), 7.55 (q, J=8.0 Hz, 1H), 7.45 (t, J=8.0 Hz, 1H), 7.38-7.27 (m, 5H), 6.84 (brs, 1H), 6.60 (s, 1H), 6.35 (s, 1H), 6.19 (d, J=16.4 Hz, 1H), 5.79 (d, J=12.0 Hz, 1H), 4.97-4.76 (m, 1H), 4.54-2.60 (m, 31H), 2.22-2.13 (m, 3H), 1.79 (brs, 5H), 1.44 (brs, 2H), 0.82 (d, J=6.8 Hz, 6H). LC-MS: m/z 1101.7 [M+H]⁺.

### Compound 299

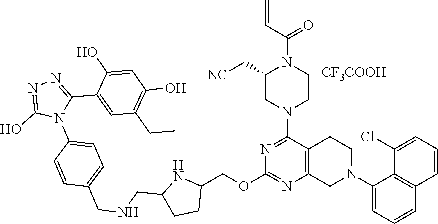 [SMILES: C=CC(=O)N1CCN(c2nc(OCC3CCC(CNCc4ccc(-n5c(O)nnc5-c5cc(CC)c(O)cc5O)cc4)N3)nc3c2CCN(c2cccc4cccc(Cl)c24)C3)C[C@@H]1CC#N ; "CF3COOH"]

### 2-((2S)-1-acryloyl-4-(7-(8-chloronaphthalen-1-yl)-2-((5-(((4-(3-(5-ethyl-2,4-dihydroxyphenyl)-5-hydroxy-4H-1,2,4-triazol-4-yl)benzyl)amino)methyl)pyrrolidin-2-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.95 (s, 1H), 9.61 (s, 1H), 9.49 (brs, 1H). 9.35 (s, 1H), 9.16 (brs, 1H), 9.09 (brs, 1H), 8.79 (brs, 1H), 7.93 (d, J=8.4 Hz, 1H), 7.76 (dd, J=8.0 Hz, 4.0 Hz, 1H), 7.58 (d, J=6.0 Hz, 1H), 7.55 (q, J=8.0 Hz, 1H), 7.47-7.43 (m, 3H), 7.35 (dd, J=19.6 Hz, 7.6 Hz, 1H), 7.27 (d, J=8.0 Hz, 2H), 6.93 (s, 1H), 6.84 (brs, 1H), 6.23 (s, 1H), 6.20 (d, J=17.2 Hz, 1H), 5.80 (d, J=11.2 Hz, 1H), 4.97-4.76 (m, 1H), 4.57-2.67 (m, 22H), 2.38 (q, J=8.0 Hz, 1H), 2.21-2.14 (m, 2H), 1.79 (brs, 2H), 1.02 (t, J=7.2 Hz, 3H). LC-MS: m/z 910.4 [M+H]⁺.

### Compound 300

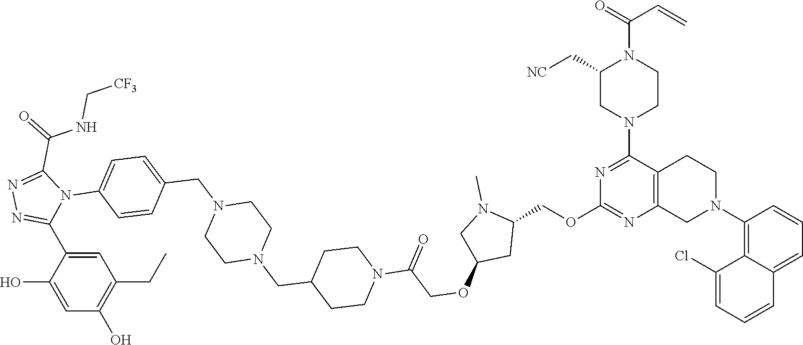 [SMILES: C=CC(=O)N1CCN(c2nc(OC[C@@H]3C[C@@H](OCC(=O)N4CCC(CN5CCN(Cc6ccc(-n7c(C(=O)NCC(F)(F)F)nnc7-c7cc(CC)c(O)cc7O)cc6)CC5)CC4)CN3C)nc3c2CCN(c2cccc4cccc(Cl)c24)C3)C[C@@H]1CC#N]

### 4-(4-((4-((1-(2-(((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)oxy)acetyl)piperidin-4-yl)methyl)piperazin-1-yl)methyl)phenyl)-5-(5-ethyl-2,4-dihydroxyphenyl)-N-(2,2,2-trifluoroethyl)-4H-1,2,4-triazole-3-carboxamide

¹H NMR (DMSO-d₆, 400 MHz): δ 10.46 (brs, 1H), 10.07 (brs, 1H), 9.72 (brs, 1H), 9.63 (t, J=6.4 Hz, 1H), 7.93 (d, J=8.0 Hz, 1H), 7.76 (dd, J=8.0, 3.2 Hz, 1H), 7.59-7.51 (m, 2H), 7.47-7.27 (m, 6H), 6.85-6.82 (m, 1H), 6.60 (s, 1H), 6.31 (s, 1H), 6.19 (d, J=16.4 Hz, 1H), 5.79 (d, J=12.0 Hz, 1H), 4.96-4.75 (m, 1H), 4.61-4.44 (m, 2H), 4.28-4.17 (m, 6H), 4.00-3.94 (m, 4H), 3.82-3.50 (m, 6H), 3.34-2.86 (m, 20H), 2.74-2.54 (m, 4H), 2.36-2.32 (m, 1H), 2.26 (q, J=7.6 Hz, 2H), 2.06-1.99 (m, 1H), 1.85-1.72 (m, 4H), 1.12-0.99 (m, 2H), 0.88 (t, J=7.6 Hz, 3H). LC-MS: m/z 1243.6 [M+H]⁺.

### Compound 301

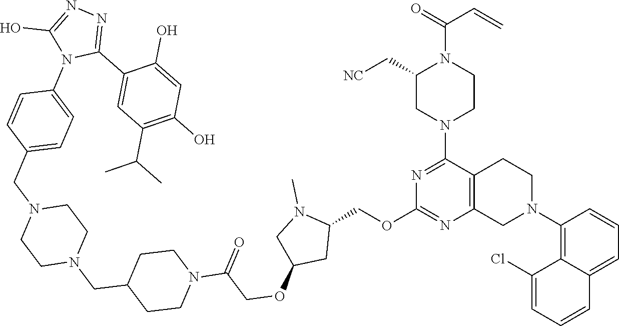 [SMILES: C=CC(=O)N1CCN(c2nc(OC[C@@H]3C[C@@H](OCC(=O)N4CCC(CN5CCN(Cc6ccc(-n7c(O)nnc7-c7cc(C(C)C)c(O)cc7O)cc6)CC5)CC4)CN3C)nc3c2CCN(c2cccc4cccc(Cl)c24)C3)C[C@@H]1CC#N]

### 2-((S)-1-acryloyl-4-(7-(8-chloronaphthalen-1-yl)-2-(((2S,4R)-4-(2-(4-((4-(4-(3-(2,4-dihydroxy-5-isopropylphenyl)-5-hydroxy-4H-1,2,4-triazol-4-yl)benzyl)piperazin-1-yl)methyl)piperidin-1-yl)-2-oxoethoxy)-1-methylpyrrolidin-2-yl)methoxy)-5,6,7,8-tetrahydropyrido[3,4-d]pyrimidin-4-yl)piperazin-2-yl)acetonitrile

¹H NMR (DMSO-d₆, 400 MHz): δ 11.95 (s, 1H), 10.00 (brs, 1H), 9.62 (s, 1H), 9.37 (s, 1H), 7.93 (d, J=7.6 Hz, 1H), 7.76 (dd, J=7.6, 3.6 Hz, 1H), 7.59-7.51 (m, 2H), 7.45 (dd, J=7.6, 8.0 Hz, 1H), 7.38-7.32 (m, 3H), 7.22-7.20 (m, 2H), 6.97-6.86 (m, 1H), 6.81 (s, 1H), 6.26 (s, 1H), 6.19 (d, J=17.2 Hz, 1H), 5.79 (d, J=11.6 Hz, 1H), 4.98-4.75 (m, 1H), 4.60-4.43 (m, 4H), 4.31-4.17 (m, 7H), 4.07-3.97 (m, 5H), 3.81-3.48 (m, 6H), 3.34-3.30 (m, 2H), 3.18-2.82 (m, 16H), 2.74-2.50 (m, 2H), 2.42-2.32 (m, 3H), 2.02-1.98 (m, 1H), 1.71-1.70 (m, 2H), 1.12-1.06 (m, 1H), 0.88 (d, J=6.8 Hz, 6H). LC-MS: m/z 1148.6 [M+H]⁺.

### Compound 302

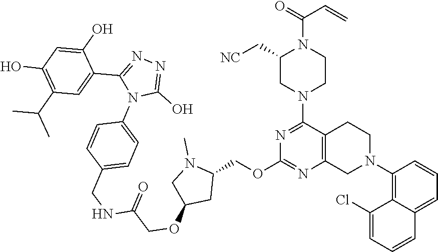 [SMILES: C=CC(=O)N1CCN(c2nc(OC[C@@H]3C[C@@H](OCC(=O)NCc4ccc(-n5c(O)nnc5-c5cc(C(C)C)c(O)cc5O)cc4)CN3C)nc3c2CCN(c2cccc4cccc(Cl)c24)C3)C[C@@H]1CC#N]

### 2-(((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)oxy)-N-(4-(3-(2,4-dihydroxy-5-isopropylphenyl)-5-hydroxy-4H-1,2,4-triazol-4-yl)benzyl)acetamide

¹H NMR (DMSO-d₆, 400 MHz): δ 11.90 (s, 1H), 9.95 (brs, 1H), 9.58 (s, 1H), 9.37 (s, 1H), 8.42 (t, J=6.0 Hz, 1H), 7.93 (d, J=8.0 Hz, 1H), 7.76 (dd, J=8.0, 3.6 Hz, 1H), 7.59-7.51 (m, 2H), 7.45 (dd, J=7.6, 8.4 Hz, 1H), 7.35 (dd, J=15.6, 7.6 Hz, 1H), 7.24 (d, J=8.4 Hz, 2H), 7.11 (d, J=8.4 Hz, 2H), 6.90-6.83 (m, 1H), 6.85 (s, 1H), 6.23 (s, 1H), 6.19 (d, J=16.4 Hz, 1H), 5.79 (d, J=11.6 Hz, 1H), 4.97-4.94 (m, 1H), 4.77-4.44 (m, 3H), 4.31-4.29 (m, 3H), 4.24-4.14 (m, 1H), 4.02-3.76 (m, 5H), 3.54-3.36 (m, 2H), 3.16-2.70 (m, 11H), 2.43-2.38 (m, 1H), 2.04-1.96 (m, 1H), 1.84-1.81 (m, 1H), 0.99 (d, J=7.2 Hz, 6H). LC-MS: m/z 982.5 [M+H]⁺.

### Compound 303

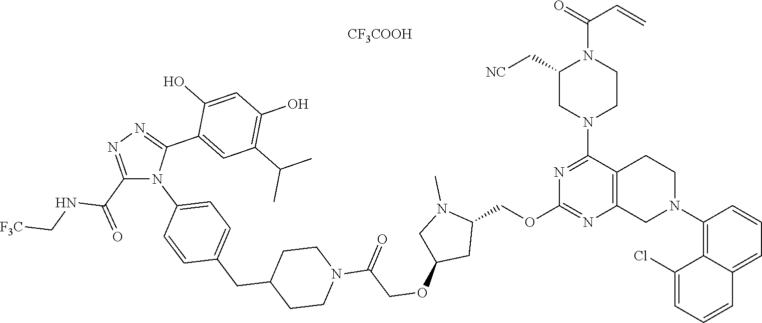 [SMILES: C=CC(=O)N1CCN(c2nc(OC[C@@H]3C[C@@H](OCC(=O)N4CCC(Cc5ccc(-n6c(C(=O)NCC(F)(F)F)nnc6-c6cc(C(C)C)c(O)cc6O)cc5)CC4)CN3C)nc3c2CCN(c2cccc4cccc(Cl)c24)C3)C[C@@H]1CC#N ; "CF3COOH"]

### 4-(4-((1-(2-(((3R,5S)-5-(((4-((S)-4-acryloyl-3-(cyanomethyl)piperazin-1-yl)-7-(8-chloronaphthalen-1-yl)-5,6,7,8-tetrahydropyrido[3,4-d]pyrimidin-2-yl)oxy)methyl)-1-methylpyrrolidin-3-yl)oxy)acetyl)piperidin-4-yl)methyl)phenyl)-5-(2,4-dihydroxy-5-isopropylphenyl)-N-(2,2,2-trifluoroethyl)-4H-1,2,4-triazole-3-carboxamide

¹H NMR (DMSO-d₆, 400 MHz): δ 9.95 (brs, 1H), 9.79 (s, 1H), 9.60 (t, J=6.4 Hz, 1H), 7.93 (d, J=7.6 Hz, 1H), 7.75 (dd, J=8.0, 3.2 Hz, 1H), 7.59-7.51 (m, 2H), 7.45 (dd, J=8.0, 7.6 Hz, 1H), 7.38-7.22 (m, 5H), 6.88-6.82 (m, 1H), 6.61 (s, 1H), 6.35 (s, 1H), 6.19 (d, J=16.4 Hz, 1H), 5.78 (d, J=12.4 Hz, 1H), 4.99-4.94 (m, 1H), 4.97-4.95 (m, 1H), 4.77-4.43 (m, 4H), 4.32-4.17 (m, 5H), 4.00-3.91 (m, 4H), 3.81-3.51 (m, 5H), 3.35-3.06 (m, 7H), 3.00 (s, 3H), 2.94-2.70 (m, 4H), 2.56-2.54 (m, 2H), 2.04-1.98 (m, 1H), 1.83-1.61 (m, 4H), 1.23-1.01 (m, 2H), 0.82 (d, J=6.8 Hz, 6H). LC-MS: m/z 1159.5 [M+H]⁺.

### Compound 304

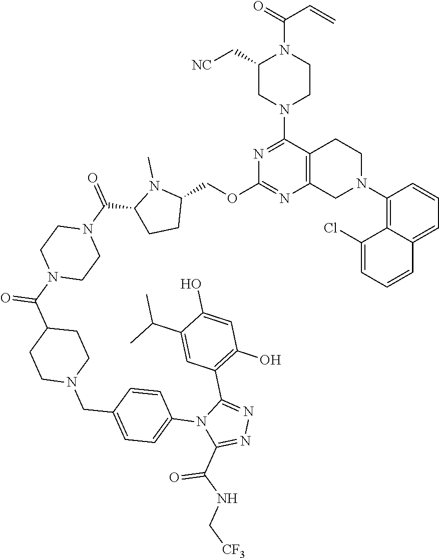 [SMILES: C=CC(=O)N1CCN(c2nc(OC[C@@H]3CC[C@H](C(=O)N4CCN(C(=O)C5CCN(Cc6ccc(-n7c(C(=O)NCC(F)(F)F)nnc7-c7cc(C(C)C)c(O)cc7O)cc6)CC5)CC4)N3C)nc3c2CCN(c2cccc4cccc(Cl)c24)C3)C[C@@H]1CC#N]

### 4-{4-[(4-{4-[(2R,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidine-2-carbonyl]piperazine-1-carbonyl}piperidin-1-yl)methyl]p-henyl}-5-(2,4-dihydroxy-5-isopropylphenyl)-N-(2,2,2-trifluoroethyl)-1,2,4-triazole-3-carboxamide

¹H NMR (CDCl₃, 400 MHz): δ 11.42 (s, 1H), 7.76-7.72 (m, 2H), 7.65-7.44 (m, 5H), 7.37-7.28 (m, 3H), 7.25-7.23 (m, 1H), 6.55-6.38 (m, 5H), 5.82 (s, 1H), 5.08-4.44 (m, 1H), 4.39-4.04 (m, 3H), 4.02-3.80 (m, 8H), 3.60-2.97 (m, 17H), 2.91-2.45 (m, 8H), 2.44-1.91 (m, 7H), 1.90-1.59 (m, 2H), 1.48-1.28 (m, 1H), 1.27 (s, 1H), 0.73 (d, J=6.8 Hz, 6H). 9F NMR (CDCl₃, 376.6 MHz) δ −72.16. LC-MS: m/z 1241.3 [M+H]⁺.

### Compound 305

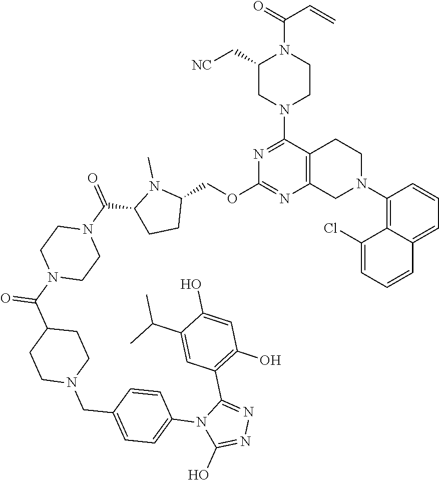 [SMILES: C=CC(=O)N1CCN(c2nc(OC[C@@H]3CC[C@H](C(=O)N4CCN(C(=O)C5CCN(Cc6ccc(-n7c(O)nnc7-c7cc(C(C)C)c(O)cc7O)cc6)CC5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-{4-[1-({4-[3-(2,4-dihydroxy-5-isopropylphenyl)-5-hydroxy-1,2,4-triazol-4-yl]phenyl}methyl)piperidine-4-carbonyl]piperazine-1-carbonyl}-1-methylpyrrolidin-2-yl]methoxy}-5H,6H,8H-pyri-do[3,4-d]pyrimidin-4-yl]-1-(prop-2-enoyl)piperazin-2-yl]acetonitrile

¹H NMR (CDCl₃, 400 MHz): δ 11.28 (s, 1H), 9.79 (s, 1H), 9.10-8.40 (m, 1H), 7.75-7.73 (m, 1H), 7.62-7.60 (m, 1H), 7.50-7.44 (m, 4H), 7.34-7.32 (m, 1H), 7.28-7.20 (m, 3H), 6.59-6.47 (m, 4H), 5.82-5.07 (m, 1H), 4.38-4.07 (m, 4H), 3.93-2.82 (m, 24H), 2.59-2.29 (m, 5H), 2.28-2.10 (m, 4H), 2.09-1.87 (m, 6H), 1.65-1.48 (m, 2H), 1.27 (s, 1H), 0.67 (d, J=6.8 Hz, 6H). LC-MS: m/z 1132.4 [M+H]⁺.

### Compound 306

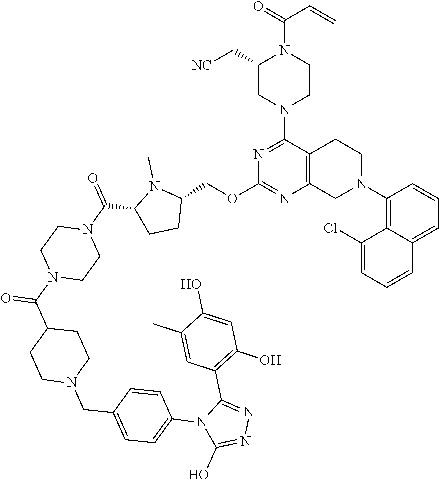 [SMILES: C=CC(=O)N1CCN(c2nc(OC[C@@H]3CC[C@H](C(=O)N4CCN(C(=O)C5CCN(Cc6ccc(-n7c(O)nnc7-c7cc(C)c(O)cc7O)cc6)CC5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-{4-[1-({4-[3-(2,4-dihydroxy-5-methylphenyl)-5-hydroxy-1,2,4-triazol-4-yl]phenyl}methyl)piperidine-4-carbonyl]piperazine-1-carbonyl}-1-methylpyrrolidin-2-yl]methoxy}-5H,6H,8H-pyrido [3,4-d]pyrimidin-4-yl]-1-(prop-2-enoyl)piperazin-2-yl]acetonitrile

¹H NMR (CDCl₃, 400 MHz): δ 10.42-9.40 (m, 1H), 7.77-7.75 (d, J=5.3 Hz, 1H), 7.64-7.60 (m, 1H), 7.54-7.41 (m, 4H), 7.36-7.32 (m, 2H), 7.28-7.02 (m, 2H), 6.61-6.48 (m, 1H), 6.41-6.37 (m, 1H), 6.36-6.28 (m, 1H), 6.27-6.18 (m, 1H), 5.84 (s, 1H), 4.43-4.39 (m, 3H), 4.29-3.79 (m, 7H), 3.74-3.27 (m, 10H), 3.26-2.71 (m, 9H), 2.64-2.40 (m, 5H), 2.07-1.88 (m, 4H), 1.87-1.77 (m, 4H), 1.76-1.61 (m, 3H), 1.60-1.28 (m, 5H). LC-MS: m/z 1104.3 [M+H]⁺.

### Compound 307

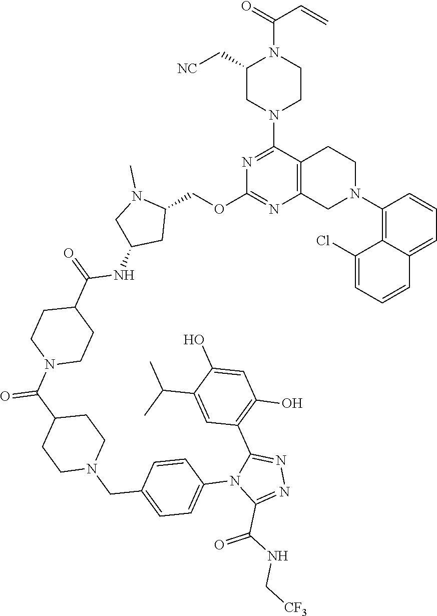 [SMILES: C=CC(=O)N1CCN(c2nc(OC[C@@H]3C[C@H](NC(=O)C4CCN(C(=O)C5CCN(Cc6ccc(-n7c(C(=O)NCC(F)(F)F)nnc7-c7cc(C(C)C)c(O)cc7O)cc6)CC5)CC4)CN3C)nc3c2CCN(c2cccc4cccc(Cl)c24)C3)C[C@@H]1CC#N]

### N-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]-1-[1-({4-[3-(2,4-dihydroxy-5-isopropylphenyl)-5-[(2,2,2-tri-fluoroethyl)carbamoyl]-1,2,4-triazol-4-yl]phenyl}methyl)piperidine-4-carbonyl]piperidine-4-carboxamide

¹H NMR (DMSO-d₆, 400 MHz): δ 10.47 (s, 1H), 9.77 (s, 1H), 9.65 (t, J=6.7 Hz, 1H), 7.93-7.91 (m, 2H), 7.80-7.59 (m, 1H), 7.57 (dd, J=7.4, 1.3 Hz, 2H), 7.44-7.38 (m, 1H), 7.36-7.30 (m, 5H), 6.85 (s, 1H), 6.62 (d, J=2.1 Hz, 1H), 6.34 (s, 1H), 6.18 (d, J=16.7 Hz, 1H), 5.77 (d, J=10.6 Hz, 1H), 4.96-4.76 (m, 1H), 4.60-4.21 (m, 4H), 4.20-3.98 (m, 7H), 3.96-3.94 (m, 1H), 3.47 (s, 3H), 3.35-3.26 (m, 2H), 3.19-3.00 (m, 4H), 2.98-2.86 (m, 3H), 2.80-2.63 (m, 4H), 2.45-2.35 (m, 5H), 2.30-2.10 (m, 3H), 2.05-1.75 (m, 3H), 1.72-1.46 (m, 7H), 1.45-1.27 (m, 2H), 0.82 (d, J=6.9 Hz, 6H). 9F NMR (DMSO-d₆, 376.6 MHz) δ −70.32. LC-MS: m/z 1255.3 [M+H]⁺.

### Compound 308

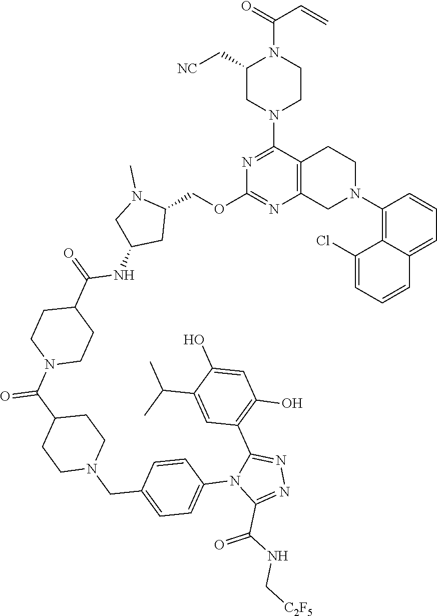

### N-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]-1-[1-({4-[3-(2,4-dihydroxy-5-isopropylphenyl)-5-[(2,2,3,3,3-pentafluoropropyl)carbamoyl]-1,2,4-triazol-4-yl]phenyl}methyl) piperidine-4-carbonyl]piperidine-4-carboxamide

¹H NMR (DMSO-d₆, 400 MHz): δ 10.47 (s, 1H), 9.77 (s, 1H), 9.65 (t, J=6.7 Hz, 1H), 7.93-7.91 (m, 1H), 7.90-7.85 (m, 1H), 7.80-7.70 (m, 1H), 7.57 (d, J=7.4 Hz, 1H), 7.55 (d, J=7.4 Hz, 1H), 7.54-7.50 (m, 1H), 7.48-7.41 (m, 3H), 7.40-7.28 (m, 2H), 6.85 (s, 1H), 6.62 (s, 1H), 6.34 (s, 1H), 6.18 (d, J=16.7 Hz, 1H), 5.77 (d, J=10.6 Hz, 1H), 4.96-4.76 (m, 1H), 4.60-4.21 (m, 4H), 4.20-3.85 (m, 7H), 3.80-3.68 (m, 1H), 3.62-3.41 (m, 4H), 3.40-3.23 (m, 5H), 3.19-3.00 (m, 3H), 2.98-2.86 (m, 4H), 2.80-2.63 (m, 3H), 2.45-2.35 (m, 3H), 2.30-2.10 (m, 3H), 2.05-1.90 (m, 2H), 1.85-1.61 (m, 3H), 1.60-1.45 (m, 4H), 1.44-1.27 (m, 2H), 0.83 (d, J=6.9 Hz, 6H). 19F NMR (DMSO-d₆, 376.6 MHz) δ −83.61, −120.21. LC-MS: m/z 1305.4 [M+H]⁺.

### Compound 309

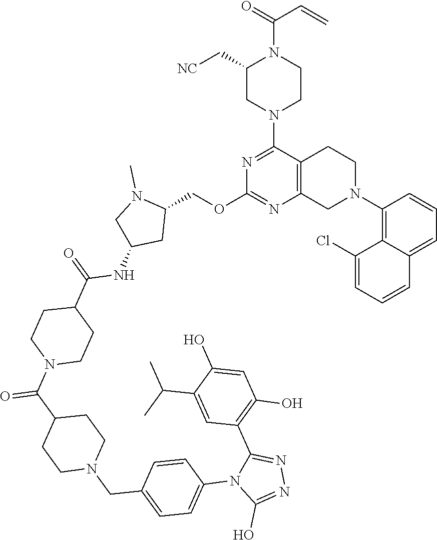 [SMILES: C=CC(=O)N1CCN(c2nc(OC[C@@H]3C[C@H](NC(=O)C4CCN(C(=O)C5CCN(Cc6ccc(-n7c(O)nnc7-c7cc(C(C)C)c(O)cc7O)cc6)CC5)CC4)CN3C)nc3c2CCN(c2cccc4cccc(Cl)c24)C3)C[C@@H]1CC#N]

### N-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]-1-[1-({4-[3-(2,4-dihydroxy-5-isopropylphenyl)-5-hydroxy-1,2,4-triazol-4-yl]phenyl}methyl)piperidine-4-carbonyl]piperidine-4-carboxamide

¹H NMR (DMSO-d₆, 400 MHz): δ 11.96 (s, 1H), 9.86-9.20 (m, 2H), 7.95-7.85 (m, 2H), 7.77 (d, J=7.4, 1H), 7.60-7.51 (m, 2H), 7.50-7.41 (m, 1H), 7.40-7.25 (m, 3H), 7.15 (d, J=7.4, 2H), 6.95-6.70 (m, 2H), 6.26 (s, 1H), 6.18 (d, J=16.7 Hz, 1H), 5.77 (d, J=10.6 Hz, 1H), 4.96-4.76 (m, 1H), 4.55-3.80 (m, 9H), 3.50-3.41 (m, 2H), 3.30-3.13 (m, 3H), 2.98-2.81 (m, 3H), 2.80-2.63 (m, 2H), 2.60-2.55 (s, 5H), 2.30-2.26 (m, 4H), 2.25-2.01 (m, 3H), 2.00-1.86 (m, 3H), 1.85-1.35 (m, 9H), 1.30-1.20 (m, 2H), 0.91 (d, J=6.9 Hz, 6H). LC-MS: m/z 1146.4 [M+H]⁺.

### Compound 310

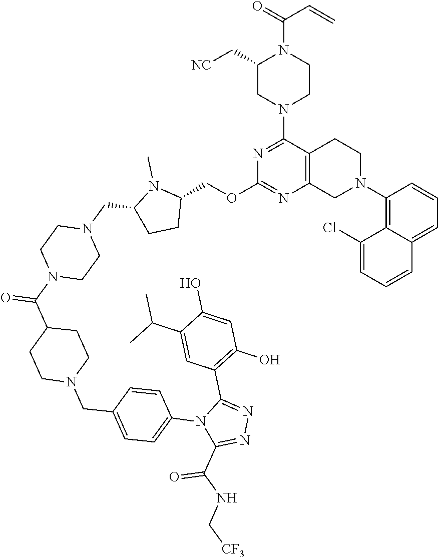 [SMILES: C=CC(=O)N1CCN(c2nc(OC[C@@H]3CC[C@H](CN4CCN(C(=O)C5CCN(Cc6ccc(-n7c(C(=O)NCC(F)(F)F)nnc7-c7cc(C(C)C)c(O)cc7O)cc6)CC5)CC4)N3C)nc3c2CCN(c2cccc4cccc(Cl)c24)C3)C[C@@H]1CC#N]

### 4-(4-{[4-(4-{[(2R,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-2-yl]methyl}piperazine-1-carbonyl)piperidin-1-yl]methyl}phenyl)-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.47 (s, 1H), 9.77 (s, 1H), 9.59 (d, J=6.6 Hz, 1H), 7.93 (d, J=8.2 Hz, 1H), 7.75 (dd, J=8.1, 4.5 Hz, 1H), 7.59 (d, J=7.4 Hz, 1H), 7.56-7.50 (m, 1H), 7.45 (t, J=7.8 Hz, 1H), 7.39-7.29 (m, 5H), 6.86 (s, 1H), 6.62 (s, 1H), 6.34 (s, 1H), 6.19 (d, J=16.7 Hz, 1H), 5.78 (d, J=10.5 Hz, 1H), 5.04-4.72 (m, 1H), 4.49-4.11 (m, 3H), 4.10-3.82 (m, 6H), 3.81-3.68 (m, 1H), 3.67-3.38 (m 8H), 3.20-2.99 (m, 5H), 2.91 (t, J=6.7 Hz, 2H), 2.82 (d, J=10.5 Hz, 3H), 2.68 (s, 2H), 2.38 (s, 6H), 2.33 (s, 2H), 2.20 (dd, J=12.0, 6.5 Hz, 1H), 1.99 (s, 2H), 1.86 (s, 2H), 1.56 (s, 6H), 0.83 (d, J=6.9 Hz, 6H). LC-MS: m/z 1227.4 [M+H]⁺.

### Compound 311

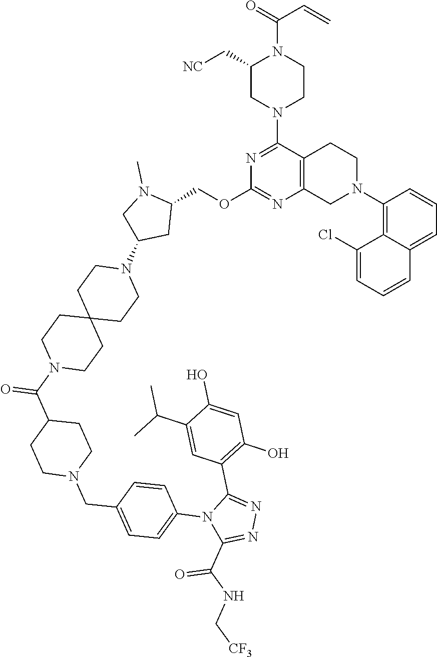 [SMILES: C=CC(=O)N1CCN(c2nc(OC[C@@H]3C[C@H](N4CCC5(CCN(C(=O)C6CCN(Cc7ccc(-n8c(C(=O)NCC(F)(F)F)nnc8-c8cc(C(C)C)c(O)cc8O)cc7)CC6)CC5)CC4)CN3C)nc3c2CCN(c2cccc4cccc(Cl)c24)C3)C[C@@H]1CC#N]

### 4-{4-[(4-{9-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]-3,9-diazaspiro[5.5]undecane-3-carbonyl}piperidin-1-yl)methyl]phenyl}-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.48 (s, 1H), 9.77 (s, 1H), 9.60 (t, J=6.5 Hz, 1H), 7.93 (d, J=8.1 Hz, 1H), 7.75 (d, J=8.2, 1H), 7.59 (d, J=7.5 Hz, 1H), 7.56 (d, J=8.0 Hz, 1H), 7.45 (t, J=7.8 Hz, 1H), 7.41-7.26 (m, 5H), 6.86 (s, 1H), 6.62 (s, 1H), 6.35 (s, 1H), 6.19 (d, J=16.6 Hz, 1H), 5.78 (d, J=10.6 Hz, 1H), 5.98-4.76 (m, 1H), 4.49-4.09 (m, 4H), 4.08-3.89 (m, 4H), 3.88-3.70 (m, 1H), 3.47 (s, 3H), 3.40 (s, 4H), 3.17 (d, J=5.0 Hz, 1H), 3.16-3.05 (m, 3H), 3.04-2.87 (m, 4H), 2.85-2.80 (m, 2H), 2.68 (p, J=1.9 Hz, 1H), 2.55 (s, 3H), 2.49-2.19 (m, 8H), 2.10-1.99 (m, 3H), 1.70-1.52 (m, 5H), 1.50-1.35 (m, 6H), 1.30 (s, 2H), 1.24 (s, 1H), 0.83 (d, J=6.9 Hz, 6H). LC-MS: m/z 1281.4 [M+H]⁺.

### Compound 312

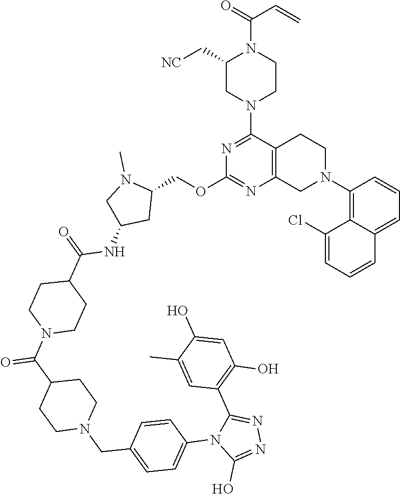 [SMILES: C=CC(=O)N1CCN(c2nc(OC[C@@H]3C[C@H](NC(=O)C4CCN(C(=O)C5CCN(Cc6ccc(-n7c(O)nnc7-c7cc(C)c(O)cc7O)cc6)CC5)CC4)CN3C)nc3c2CCN(c2cccc4cccc(Cl)c24)C3)C[C@@H]1CC#N]

### N-[(3S,5S)-5-({[7-(8-chloronaphthalen-1-yl)-4-[(3S)-3-(cyanomethyl)-4-(prop-2-e-noyl)piperazin-1-yl]-5H,6H,8H-pyrido[3,4-d]pyrimidin-2-yl]oxy}methyl)-1-meth-ylpyrrolidin-3-yl]-1-[1-({4-[3-(2,4-dihydroxy-5-methylphenyl)-5-hydroxy-1,2,4-tri azol-4-yl]phenyl}methyl)piperidine-4-carbonyl]piperidine-4-carboxamide

¹H NMR (DMSO-d₆, 400 MHz): δ 11.96 (s, 1H), 9.55 (s, 1H), 9.35 (s, 1H), 7.91 (d, J=8.4, 1H), 7.85 (d, J=7.8, 1H), 7.75 (d, J=7.7, 1H), 7.60-7.51 (m, 2H), 7.50-7.41 (m, 1H), 7.40-7.30 (m, 1H), 7.28-7.20 (m, 2H), 7.12 (d, J=8.5, 2H), 6.85 (s, 2H), 6.23-6.15 (m, 2H), 5.77 (d, J=10.6 Hz, 1H), 4.96-4.76 (m, 1H), 4.60-4.41 (m, 2H), 4.40-4.21 (m, 2H), 4.20-4.05 (m, 3H), 4.00-3.85 (m, 1H) 3.80-3.65 (m, 2H), 3.60-358 (m, 2H), 3.52-3.41 (m, 2H), 3.40-3.23 (m, 2H), 3.19-3.03 (m, 3H), 3.00-2.95 (m, 1H), 2.80-2.63 (m, 2H), 2.45-2.30 (m, 4H), 2.25-2.16 (m, 1H), 2.15-2.07 (m, 3H), 2.05-1.80 (m, 6H), 1.78-1.65 (m, 1H), 1.63-1.51 (m, 1H), 1.50-1.41 (m, 3H), 1.40-1.36 (m, 5H), 1.35-1.28 (m, 2H). LC-MS: m/z 1118.3 [M+H]⁺.

### Compound 313

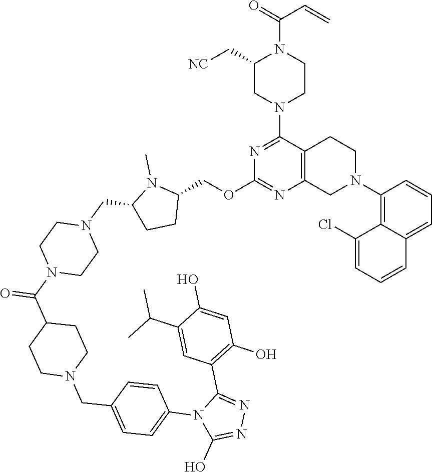 [SMILES: C=CC(=O)N1CCN(c2nc(OC[C@@H]3CC[C@H](CN4CCN(C(=O)C5CCN(Cc6ccc(-n7c(O)nnc7-c7cc(C(C)C)c(O)cc7O)cc6)CC5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-({4-[1-({4-[3-(2,4-dihydroxy-5-isopropylphenyl)-5-hydroxy-1,2,4-triazol-4-yl]phenyl}methyl)piperidine-4-carbonyl]piperazin-1-yl}methyl)-1-methylpyrrolidin-2-yl]methoxy}-5H,6H,8H-pyrido [3,4-d]pyrimidin-4-yl]-1-(prop-2-enoyl) piperazin-2-yl]acetonitrile

¹H NMR (DMSO-d₆, 400 MHz): δ 11.93 (s, 1H), 9.60 (s, 1H), 9.42 (s, 1H), 7.93 (d, J=8.1 Hz, 1H), 7.75 (dd, J=8.3, 4.5 Hz, 1H), 7.62-7.57 (m, 1H), 7.56-7.50 (m, 1H), 7.45 (t, J=7.8 Hz, 1H), 7.34 (dd, J=18.6, 7.6 Hz, 1H), 7.29 (d, J=8.2 Hz, 2H), 7.13 (d, J=8.1 Hz, 2H), 6.76 (s, 2H), 6.27 (s, 1H), 6.19 (d, J=16.5 Hz, 1H), 5.78 (d, J=10.7 Hz, 1H), 5.02-4.83 (m, 1H), 4.51-3.94 (m, 7H), 3.88-3.57 (m, 2H), 3.42 (s, 8H), 3.10-2.85 (m, 8H), 2.83-2.73 (m, 2H), 2.68 (p, J=1.9 Hz, 1H), 2.37 (s, 6H), 2.35-2.27 (m, 2H), 2.19 (dd, J=12.5, 6.7 Hz, 1H), 1.96 (s, 2H), 1.85 (s, 2H), 1.54 (s, 6H), 0.94 (d, J=7.0 Hz, 6H). LC-MS: m/z 1118.3 [M+H]⁺.

### Compound 314

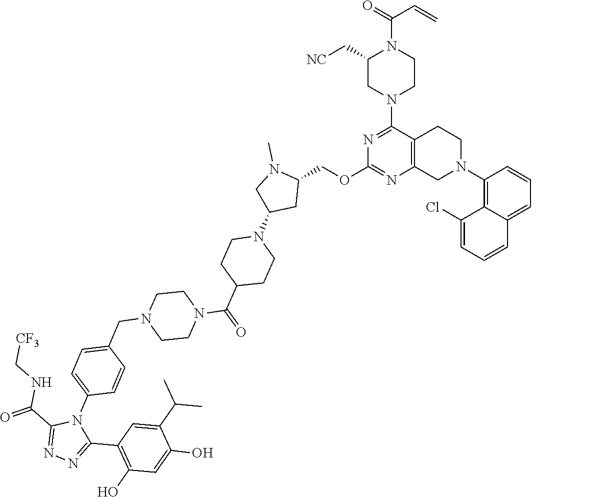 [SMILES: C=CC(=O)N1CCN(c2nc(OC[C@@H]3C[C@H](N4CCC(C(=O)N5CCN(Cc6ccc(-n7c(C(=O)NCC(F)(F)F)nnc7-c7cc(C(C)C)c(O)cc7O)cc6)CC5)CC4)CN3C)nc3c2CCN(c2cccc4cccc(Cl)c24)C3)C[C@@H]1CC#N]

### 4-{4-[(4-{1-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]piperidine-4-carbonyl}piperazin-1-yl)methyl]phenyl}-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.45 (s, 1H), 9.78 (s, 1H), 9.55 (s, 1H), 7.92 (d, J=8.1 Hz, 1H), 7.75 (dd, J=8.2, 4.3 Hz, 1H), 7.60-7.50 (m, 2H), 7.45 (t, J=7.8 Hz, 1H), 7.34-7.31 (m, 5H), 6.85 (s, 1H), 6.63 (s, 1H), 6.34 (s, 1H), 6.19 (d, J=16.7 Hz, 1H), 5.78 (d, J=10.7 Hz, 1H), 4.95-4.74 (m, 1H), 4.70-4.51 (m, 1H), 4.46-4.25 (m, 2H), 4.21-4.12 (m, 2H), 4.06-3.92 (m, 4H), 3.83-3.69 (m, 2H), 3.65-3.45 (m, 2H), 3.40-3.24 (m, 4H), 3.23-3.18 (m, 2H), 3.15-3.05 (m, 3H), 3.04-2.95 (m, 2H), 2.89-2.71 (m, 3H), 2.65-2.45 (m, 4H), 2.43-2.35 (m, 2H), 2.34-2.26 (m, 4H), 2.07-1.75 (m, 4H), 1.59-1.39 (m, 5H), 1.25 (m, 1H), 0.85 (d, J=6.9 Hz, 6H). LC-MS: m/z 1213.3 [M+H]⁺.

### Compound 315

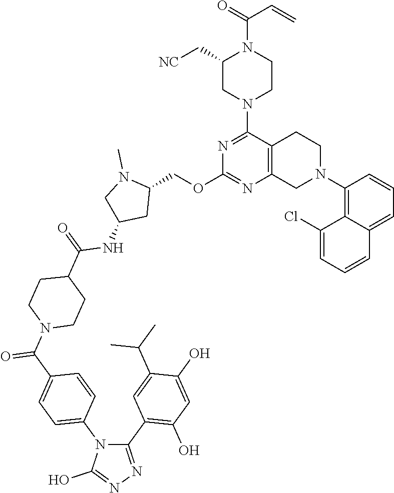 [SMILES: C=CC(=O)N1CCN(c2nc(OC[C@@H]3C[C@H](NC(=O)C4CCN(C(=O)c5ccc(-n6c(O)nnc6-c6cc(C(C)C)c(O)cc6O)cc5)CC4)CN3C)nc3c2CCN(c2cccc4cccc(Cl)c24)C3)C[C@@H]1CC#N]

### N-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]-1-{4-[3-(2,4-dihydroxy-5-isopropylphenyl)-5-hydroxy-1,2,4-triazol-4-yl]benzoyl}piperidine-4-carboxamide

¹H NMR (DMSO-d₆, 400 MHz): δ 11.97 (s, 1H), 9.60 (s, 1H), 9.38 (s, 1H), 7.92-7.85 (m, 2H), 7.75 (t, J=7.8 Hz, 1H), 7.58 (dd, J=8.2, 4.3 Hz, 2H), 7.55 (t, J=7.8 Hz, 1H), 7.42-7.31 (m, 3H), 7.30-7.20 (m, 2H), 6.86 (s, 2H), 6.21 (s, 1H), 6.16 (d, J=16.7 Hz, 1H), 5.78 (d, J=10.7 Hz, 1H), 4.95-4.74 (m, 1H), 4.50-4.35 (m, 1H), 4.34-3.90 (m, 7H), 3.80-3.70 (m, 1H), 3.60-3.45 (m, 2H), 3.40-3.24 (m, 3H), 3.20-2.81 (m, 8H), 2.80-2.70 (m, 2H), 2.55-2.30 (m, 3H), 2.24-2.20 (m, 3H), 1.80-1.40 (m, 5H), 0.99 (d, J=6.9 Hz, 6H). LC-MS: m/z 1049.2 [M+H]⁺.

### Compound 316

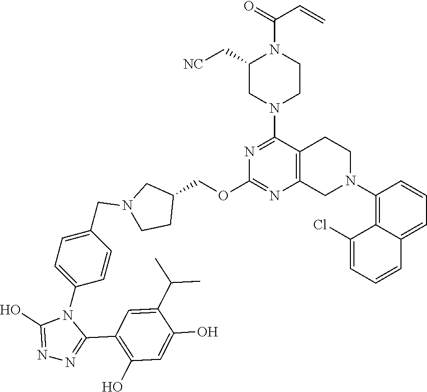 [SMILES: C=CC(=O)N1CCN(c2nc(OC[C@@H]3CCN(Cc4ccc(-n5c(O)nnc5-c5cc(C(C)C)c(O)cc5O)cc4)C3)nc3c2CCN(c2cccc4cccc(Cl)c24)C3)C[C@@H]1CC#N]

### 2-[(2S)-4-[7-(8-chloronaphthalen-1-yl)-2-{[(3R)-1-({4-[3-(2,4-dihydroxy-5-isopropylphenyl)-5-hydroxy-1,2,4-triazol-4-yl]phenyl}methyl)pyrrolidin-3-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.92 (s, 1H), 9.58 (s, 1H), 9.41 (s, 1H), 7.93 (d, J=8.1 Hz, 1H), 7.75 (dd, J=8.1, 4.0 Hz, 1H), 7.63-7.50 (m, 2H), 7.45 (t, J=7.7 Hz, 1H), 7.42-7.22 (m, 3H), 7.15-7.07 (m, 2H), 6.84 (s, 1H), 6.76 (s, 1H), 6.26 (s, 1H), 6.23-6.15 (m, 1H), 5.78 (d, J=10.7 Hz, 1H), 4.98-4.75 (m, 1H), 4.34-3.94 (m, 7H), 3.55 (s, 1H), 3.50 (d, J=10.4 Hz, 1H), 3.18-2.82 (m, 6H), 2.07-1.84 (m, 3H), 1.48 (s, 1H), 1.24 (s, 6H), 0.99-0.81 (m, 6H). LC-MS: m/z 895.2 [M+H]⁺.

### Compound 317

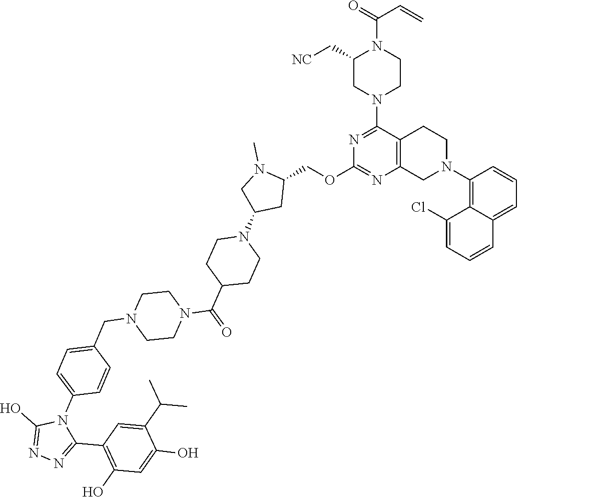 [SMILES: C=CC(=O)N1CCN(c2nc(OC[C@@H]3C[C@H](N4CCC(C(=O)N5CCN(Cc6ccc(-n7c(O)nnc7-c7cc(C(C)C)c(O)cc7O)cc6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{4-[4-({4-[3-(2,4-dihydroxy-5-isopropylphenyl)-5-hydroxy-1,2,4-triazol-4-yl]phenyl}methyl)piperazine-1-carbonyl]piperidin-1-yl}-1-methylpyrrolidin-2yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.93 (s, 1H), 9.60 (s, 1H), 9.40 (s, 1H), 7.92 (d, J=8.1 Hz, 1H), 7.75 (dd, J=8.2, 4.3 Hz, 1H), 7.60-7.50 (m, 2H), 7.45 (t, J=7.8 Hz, 1H), 7.28-7.20 (m, 3H), 7.14 (d, J=8.0 Hz, 2H), 6.85 (d, J=8.5 Hz, 1H), 6.78 (s, 1H), 6.27 (s, 1H), 6.19 (d, J=16.7 Hz, 1H), 5.78 (d, J=10.7 Hz, 1H), 4.98-4.76 (m, 1H), 4.46-4.25 (m, 1H), 4.21-4.12 (m, 2H), 4.06-3.92 (m, 2H), 3.83-3.69 (m, 1H), 3.45-3.42 (m, 7H), 3.20-3.04 (m, 4H), 2.98-2.94 (m, 4H), 2.89-2.71 (m, 3H), 2.27 (d, J=3.8 Hz, 6H), 2.07 (s, 4H), 1.54 (s, 6H), 1.28-1.11 (m, 3H), 1.08 (s, 1H), 0.95 (d, J=6.9 Hz, 6H), 0.89-0.80 (m, 1H). LC-MS: m/z 1104.3 [M+H]⁺.

### Compound 318

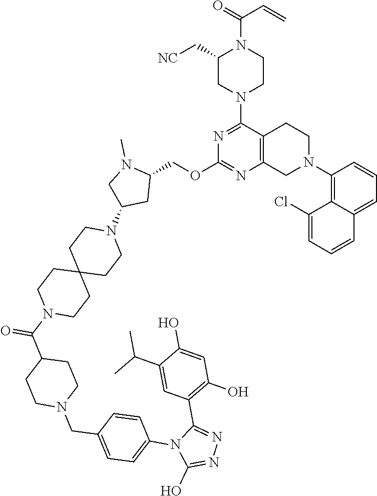 [SMILES: C=CC(=O)N1CCN(c2nc(OC[C@@H]3C[C@H](N4CCC5(CCN(C(=O)C6CCN(Cc7ccc(-n8c(O)nnc8-c8cc(C(C)C)c(O)cc8O)cc7)CC6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{9-[1-({4-[3-(2,4-dihydroxy-5-isopropylphenyl)-5-hydroxy-1,2,4-triazol-4-yl]phenyl}methyl)piperidine-4-car bonyl]-3,9-diazaspiro[5.5]undecan-3-yl}-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): 11.93 (s, 1H), 9.60 (s, 1H), 9.42 (s, 1H), 7.93 (d, J=8.1 Hz, 1H), 7.75 (dd, J=8.2, 4.2 Hz, 1H), 7.59-7.51 (m, 2H), 7.45 (t, J=7.8 Hz, 1H), 7.40-7.25 (m, 3H), 7.13 (d, J=8.1 Hz, 2H), 6.85 (s, 1H), 6.76 (s, 1H), 6.27 (s, 1H), 6.19 (d, J=16.6 Hz, 1H), 5.78 (d, J=10.6 Hz, 1H), 4.97-4.77 (m, 1H), 4.55-3.89 (m, 6H), 3.86-3.45 (m, 3H), 3.39 (s, 6H), 3.19-2.89 (m, 8H), 2.80-2.77 (m, 2H), 2.45-2.41 (m, 2H) 2.31-2.22 (m, 5H), 2.19-1.89 (m, 4H), 1.53 (d, J=8.5 Hz, 5H), 1.42 (s, 4H), 1.36-1.25 (m, 5H), 1.24 (s, 2H), 0.94 (d, J=6.9 Hz, 6H), 0.89-0.85 (m, 1H). LC-MS: m/z 1172.4 [M+H]⁺.

### Compound 319

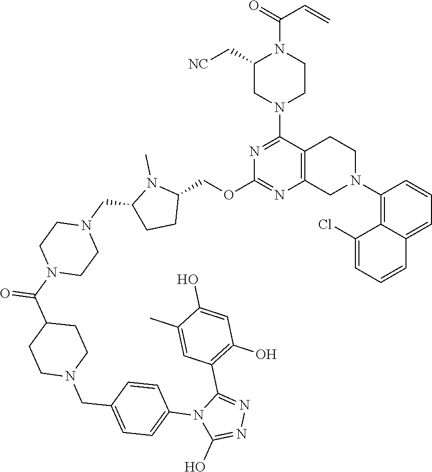 [SMILES: C=CC(=O)N1CCN(c2nc(OC[C@@H]3CC[C@H](CN4CCN(C(=O)C5CCN(Cc6ccc(-n7c(O)nnc7-c7cc(C)c(O)cc7O)cc6)CC5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-({4-[1-({4-[3-(2,4-dihydroxy-5-methylphenyl)-5-hydroxy-1,2,4-triazol-4-yl]phenyl}methyl)piperidine-4-carbonyl]piperazin-1-yl}methyl)-1-methylpyrrolidin-2-yl]methoxy}-5H,6H,8H-pyrido [3,4-d]pyrimidin-4-yl]-1-(prop-2-enoyl)piperazin-2-yl]acetonitrile

¹H NMR (DMSO-d₆, 400 MHz): δ 11.87 (s, 1H), 9.54 (s, 1H), 9.31 (s, 1H), 7.92 (d, J=8.1 Hz, 1H), 7.75 (dd, J=8.1, 4.5 Hz, 1H), 7.62-7.50 (m, 2H), 7.45 (t, J=7.8 Hz, 1H), 7.35 (dd, J=18.3, 7.4 Hz, 1H), 7.25 (d, J=8.3 Hz, 2H), 7.10 (d, J=8.4 Hz, 2H), 6.88 (s, 2H), 6.27-6.13 (m, 2H), 5.78 (d, J=11.2 Hz, 1H), 5.03-4.77 (m, 1H), 4.31-4.10 (m, 3H), 4.00 (d, J=10.7 Hz, 4H), 3.83-3.55 (m, 3H), 3.41 (s, 5H), 3.14-2.82 (m, 6H), 2.77 (d, J=10.7 Hz, 2H), 2.37 (t, J=1.6 Hz, 7H), 2.28-2.16 (m, 4H), 1.94 (s, 5H), 1.85 (s, 2H), 1.54 (s, 6H), 1.24 (s, 2H). LC-MS: m/z 1090.3 [M+H]⁺.

### Compound 320

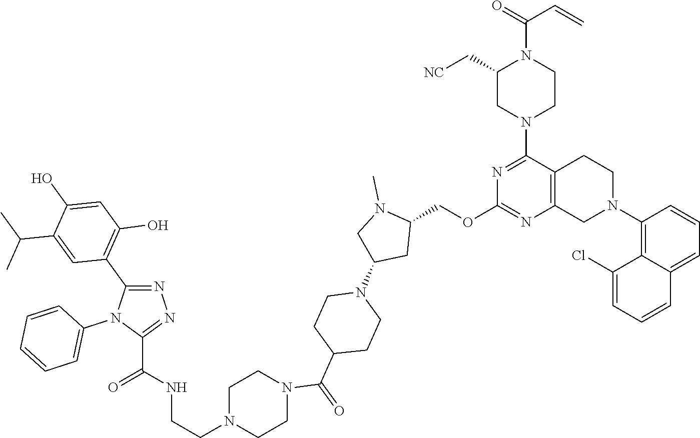 [SMILES: C=CC(=O)N1CCN(c2nc(OC[C@@H]3C[C@H](N4CCC(C(=O)N5CCN(CCNC(=O)c6nnc(-c7cc(C(C)C)c(O)cc7O)n6-c6ccccc6)CC5)CC4)CN3C)nc3c2CCN(c2cccc4cccc(Cl)c24)C3)C[C@@H]1CC#N]

### N-[2-(4-{1-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]piperidine-4-carbonyl}piperazin-1-yl)ethyl]-5-(2,4-dihy-droxy-5-isopropylphenyl)-4-phenyl-1,2,4-triazole-3-carboxamide

¹H NMR (DMSO-d₆, 400 MHz): δ 10.40 (s, 1H), 9.72 (s, 1H), 8.80 (t, J=5.8 Hz, 1H), 7.92 (dd, J=8.4, 1.2 Hz, 1H), 7.75 (dd, J=8.0, 4.4 Hz, 1H), 7.58 (d, J=7.6 Hz, 1H), 7.53 (t, J=7.6 Hz, 1H), 7.47-7.42 (m, 4H), 7.38-7.32 (m, 3H), 6.85 (s, 1H), 6.60 (s, 1H), 6.33 (s, 1H), 6.19 (d, J=16.6 Hz, 1H), 5.78 (d, J=11.5 Hz, 1H), 4.97-4.77 (m, 1H), 4.31-4.28 (m, 1H), 4.24-3.87 (m, 5H), 3.86-3.77 (m, 1H), 3.65-3.45 (m, 2H), 3.41 (s, 4H), 3.28 (s, 4H), 3.06-2.94 (m, 4H), 2.93-2.86 (m, 5H), 2.83 (d, J=10.7 Hz, 3H), 2.42 (s, 3H), 2.34 (s, 2H), 2.30 (s, 2H), 2.27 (s, 3H), 2.10-1.96 (m, 3H), 1.54 (s, 5H), 0.84 (d, J=6.9 Hz, 6H). LC-MS: m/z 1145.3 [M+H]⁺.

### Compound 321

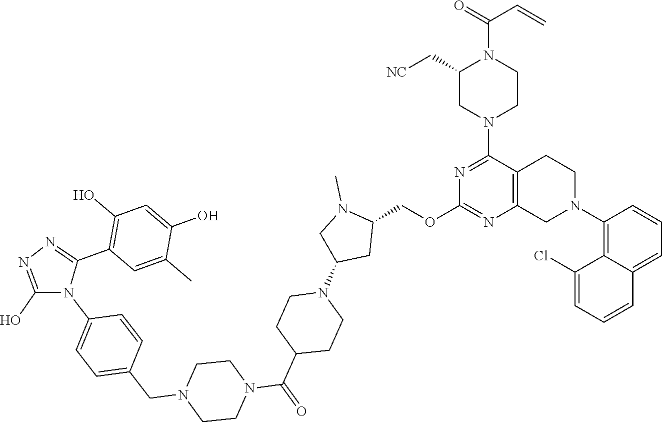 [SMILES: C=CC(=O)N1CCN(c2nc(OC[C@@H]3C[C@H](N4CCC(C(=O)N5CCN(Cc6ccc(-n7c(O)nnc7-c7cc(C)c(O)cc7O)cc6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{4-[4-({4-[3-(2,4-dihydroxy-5-methylphenyl)-5-hydroxy-1,2,4-triazol-4-yl]phenyl}methyl)piperazine-1-carbonyl]piperidin-1-yl}-1-methylpyrrolidin-2-yl]methoxy}-5H,6H,8H-pyrido[3,4d]-pyrimidin-4-yl]-1-(prop-2-enoyl)piperazin-2-yl]acetonitrile

¹H NMR (DMSO-d₆, 400 MHz): δ 11.88 (s, 1H), 9.56 (s, 1H), 9.32 (s, 1H), 7.92 (d, J=8.1 Hz, 1H), 7.74 (dd, J=8.2, 4.1 Hz, 1H), 7.63-7.50 (m, 2H), 7.45 (t, J=7.8 Hz, 1H), 7.41-7.22 (m, 3H), 7.11 (d, J=8.0 Hz, 2H), 6.90-6.84 (m, 2H), 6.27-6.14 (m, 2H), 5.78 (d, J=10.5 Hz, 1H), 4.97-4.77 (m, 1H), 4.60-4.41 (m, 1H), 4.31-4.27 (m, 1H), 4.21-4.12 (m, 2H), 4.06-3.92 (m, 2H), 3.83-3.69 (m, 2H), 3.42 (s, 6H), 3.20-3.04 (m, 4H), 2.98-2.94 (m, 4H), 2.89-2.71 (m, 3H), 2.27 (d, J=3.8 Hz, 6H), 1.95 (s, 6H), 1.54 (s, 6H), 1.35-1.06 (m, 3H), 0.94-0.86 (m, 1H). LC-MS: m/z 1076.3 [M+H]⁺.

### Compound 322

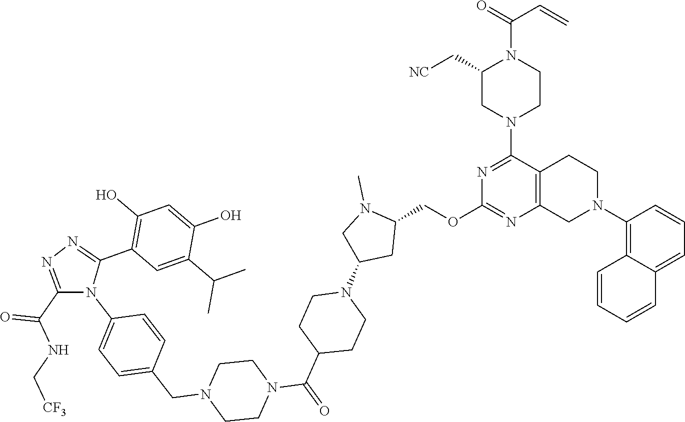 [SMILES: C=CC(=O)N1CCN(c2nc(OC[C@@H]3C[C@H](N4CCC(C(=O)N5CCN(Cc6ccc(-n7c(C(=O)NCC(F)(F)F)nnc7-c7cc(C(C)C)c(O)cc7O)cc6)CC5)CC4)CN3C)nc3c2CCN(c2cccc4ccccc24)C3)C[C@@H]1CC#N]

### 4-{4-[(4-{1-[(3S,5S)-5-[({4-[(3S)-3-(cyanomethyl)-4-(prop-2-enoyl)piperazin-1-yl]-7-(naphthalen-1-yl)-5H,6H,8H-pyrido[3,4-d]pyrimidin-2-yl}oxy)methyl]-1-methylpyrrolidin-3-yl]piperidine-4-carbonyl}piperazin-1-yl)methyl]phenyl}-5-(2,4-dihydroxy-5-isopropylphenyl)-N-(2,2,2-trifluoroethyl)-1,2,4-triazole-3-carboxamide

¹H NMR (CDCl₃, 400 MHz): δ 11.34 (s, 1H), 8.24-8.21 (m, 1H), 7.89-7.86 (m, 1H), 7.74-7.71 (t, J=6.7 Hz, 1H), 7.63 (d, J=8.2 Hz, 1H), 7.56-7.50 (m, 4H), 7.45 (t, J=7.8 Hz, 1H), 7.34 (d, J=8.0 Hz, 2H), 7.17 (d, J=7.2 Hz 1H), 6.55 (d, J=8.5 Hz, 3H), 6.43 (d, J=16.6 Hz, 1H), 5.85 (d, J=10.0 Hz, 1H), 5.16-4.39 (m, 2H), 4.28 (s, 3H), 4.12-3.95 (m, 4H), 3.63 (s, 6H), 3.57-3.29 (m, 7H), 3.18 (s, 3H), 3.06-2.72 (m, 8H), 2.50 (s, 8H), 0.78 (dd, J=6.9, 1.8 Hz, 7H). LC-MS: m/z 1179.4 [M+H]⁺.

### Compound 323

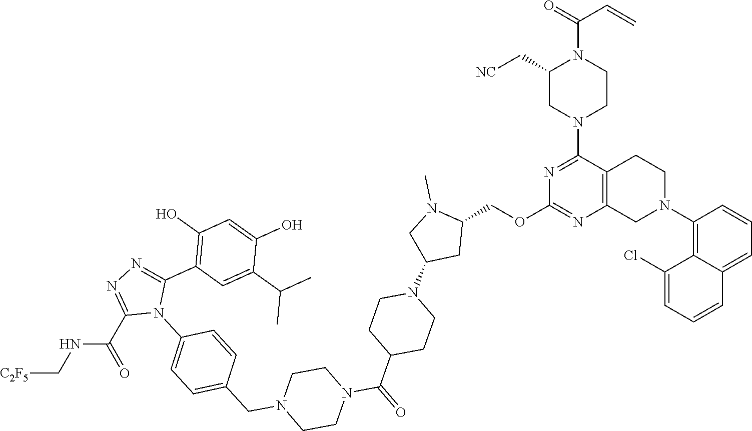

### 4-{4-[(4-{1-[(3S,5S)-5-[({4-[(3S)-3-(cyanomethyl)-4-(prop-2-enoyl)piperazin-1-yl]-7-(naphthalen-1-yl)-5H,6H,8H-pyrido[3,4-d]pyrimidin-2-yl}oxy)methyl]-1-methylpyrrolidin-3-yl]piperidine-4-carbonyl}piperazin-1-yl)methyl]phenyl}-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.37 (s, 1H), 9.76 (s, 1H), 9.59 (t, J=6.5 Hz, 1H), 7.92 (d, J=8.0 Hz, 1H), 7.75 (dd, J=8.3, 4.2 Hz, 1H), 7.62-7.49 (m, 2H), 7.45 (t, J=7.8 Hz, 1H), 7.35 (d, J=8.6 Hz, 4H), 6.85 (s, 1H), 6.65 (s, 1H), 6.34 (s, 1H), 6.19 (d, J=16.6 Hz, 1H), 5.78 (d, J=10.8 Hz, 1H), 4.97-4.77 (m, 1H), 4.50-4.11 (m, 4H), 4.03-3.98 (m, 4H), 3.75-3.71 (m, 2H), 3.53-3.41 (m, 8H), 3.33-2.70 (m, 12H), 2.38 (s, 3H), 2.30-2.25 (m, 6H), 1.91 (s, 3H), 1.55 (s, 6H). LC-MS: m/z 1263.3 [M+H]⁺.

### Compound 324

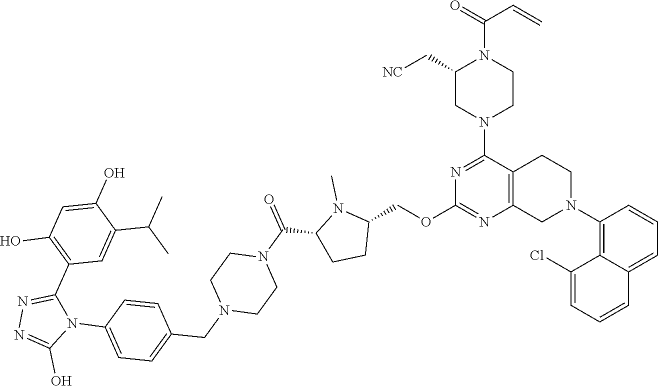 [SMILES: C=CC(=O)N1CCN(c2nc(OC[C@@H]3CC[C@H](C(=O)N4CCN(Cc5ccc(-n6c(O)nnc6-c6cc(C(C)C)c(O)cc6O)cc5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-[4-({4-[3-(2,4-dihydroxy-5-isopropylphenyl)-5-hydroxy-1,2,4-triazol-4-yl]phenyl}methyl)piperazine-1-carbonyl]-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.93 (s, 1H), 9.59 (s, 1H), 9.40 (s, 1H), 7.92 (d, J=8.1 Hz, 1H), 7.74 (dd, J=8.1, 4.8 Hz, 1H), 7.59-7.60 (m, 2H), 7.44 (dd, J=7.8, 4.0 Hz, 1H), 7.37-7.28 (m, 3H), 7.13 (dd, J=8.4, 2.0 Hz, 2H), 6.85 (s, 1H), 6.77 (s, 1H), 6.27 (s, 1H), 6.18 (d, J=16.4 Hz, 1H), 5.77 (d, J=10.4 Hz, 1H), 4.96-4.77 (m, 1H), 4.40-3.94 (m, 6H), 3.79-3.68 (m, 3H), 3.52-3.46 (m, 3H), 3.43 (s, 3H), 3.30-3.28 (m, 4H), 3.14-2.93 (m, 7H), 2.28 (d, J=3.4 Hz, 2H), 1.97 (d, J=7.2 Hz, 2H), 1.67 (s, 2H), 0.94 (d, J=6.9 Hz, 6H). LC-MS: m/z 1021.3 [M+H]⁺.

### Compound 325

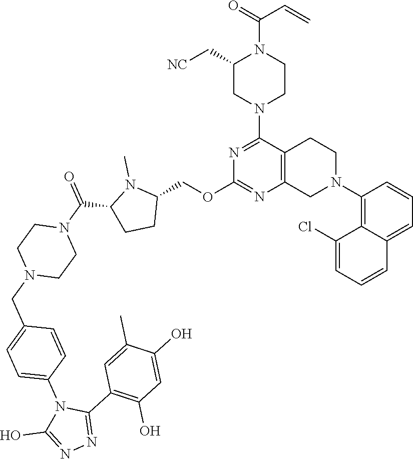 [SMILES: C=CC(=O)N1CCN(c2nc(OC[C@@H]3CC[C@H](C(=O)N4CCN(Cc5ccc(-n6c(O)nnc6-c6cc(C)c(O)cc6O)cc5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-[4-({4-[3-(2,4-dihydroxy-5-methylphenyl)-5-hydroxy-1,2,4-triazol-4-yl]phenyl}methyl)piperazine-1-carbonyl]-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.89 (s, 1H), 9.56 (s, 1H), 9.32 (s, 1H), 7.93 (d, J=8.2 Hz, 1H), 7.75 (t, J=6.6 Hz, 1H), 7.59-7.50 (m, 2H), 7.45 (t, J=7.7 Hz, 1H), 7.32 (d, J=35.6 Hz, 3H), 7.12 (s, 2H), 6.89 (s, 1H), 6.24 (s, 1H), 6.19 (d, J=16.6 Hz, 1H), 5.78 (d, J=10.3 Hz, 1H), 4.99-4.65 (m, 3H), 4.37-3.75 (m, 7H), 3.50-3.42 (m, 5H), 3.12-3.09 (m, 5H), 2.92 (s, 3H), 2.34 (p, J=1.9 Hz, 3H), 2.27 (s, 4H), 1.95-1.91 (m, 5H), 1.84 (s, 2H), 1.70-1.67 (m, 1H), 1.25-1.24 (m, 1H), 1.18 (t, J=7.3 Hz, 1H). LC-MS: m/z 993.2 [M+H]⁺.

### Compound 326

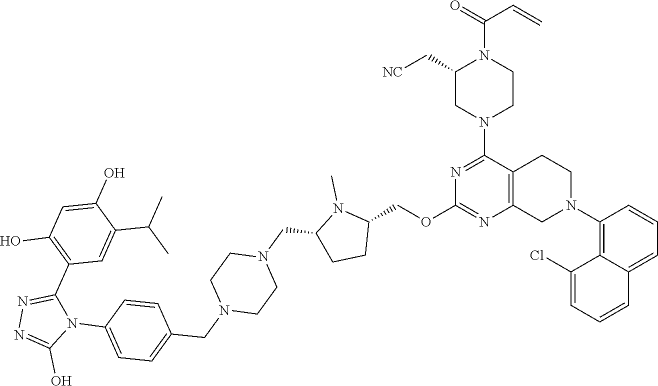 [SMILES: C=CC(=O)N1CCN(c2nc(OC[C@@H]3CC[C@H](CN4CCN(Cc5ccc(-n6c(O)nnc6-c6cc(C(C)C)c(O)cc6O)cc5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-{[4-({4-[3-(2,4-dihydroxy-5-isopropylphenyl)-5-hydroxy-1,2,4-triazol-4-yl]-phenyl}methyl)piperazin-1-yl]methyl}-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.93 (s, 1H), 9.59 (s, 1H), 9.41 (s, 1H), 7.93 (d, J=8.2 Hz, 1H), 7.74 (s, 1H), 7.65-7.49 (m, 2H), 7.45 (t, J=7.8 Hz, 1H), 7.38-7.20 (m, 3H), 7.12 (d, J=7.9 Hz, 2H), 6.76 (s, 2H), 6.26 (s, 1H), 6.19 (d, J=16.6 Hz, 1H), 5.78 (d, J=10.6 Hz, 1H), 5.13-4.79 (m, 1H), 4.47-3.91 (m, 6H), 3.89-3.45 (m, 3H), 3.41 (s, 2H), 3.21-2.86 (m, 8H), 2.68 (s, 2H), 2.40-2.23 (m, 12H), 2.22-2.09 (m, 1H), 1.83 (s, 2H), 1.52-1.35 (m, 2H), 0.94 (d, J=6.9 Hz, 6H). LC-MS: m/z 1007.2 [M+H]⁺.

### Compound 327

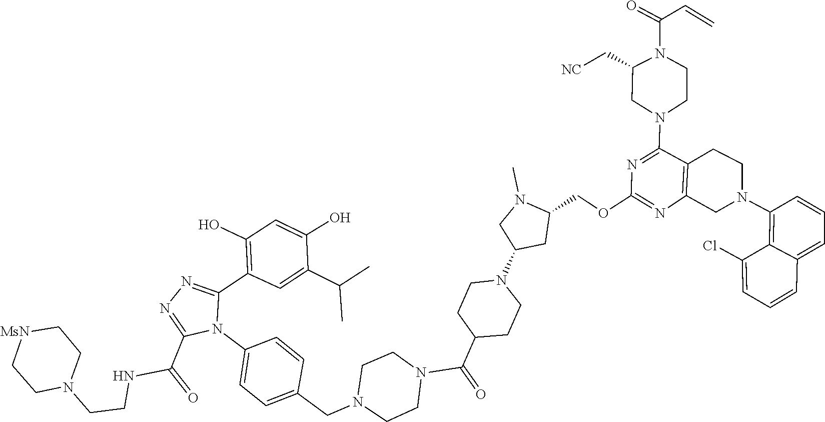

### 4-{4-[(4-{1-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]piperidine-4-carbonyl}piperazin-1-yl)methyl]phenyl}-5-(2,4-dihydroxy-5-isopropylphenyl)-N-[2-(4-methanesulfonylpiperazin-1-yl)ethyl]-1,2,4-triazole-3-carboxamide

¹H NMR (DMSO-d₆, 400 MHz): δ 10.50 (s, 1H), 9.73 (s, 1H), 8.78 (t, J=5.9 Hz, 1H), 7.92 (d, J=8.3 Hz, 1H), 7.75 (dd, J=8.1, 4.3 Hz, 1H), 7.62-7.49 (m, 2H), 7.45 (t, J=7.8 Hz, 1H), 7.41-7.31 (m, 5H), 6.86 (s, 1H), 6.60 (s, 1H), 6.34 (s, 1H), 6.19 (d, J=16.7 Hz, 1H), 5.77 (d, J=10.9 Hz, 1H), 4.97-4.77 (m, 1H), 4.32-4.13 (m, 4H), 4.04-4.00 (m, 2H), 3.87-3.65 (m, 2H), 3.52 (s, 7H), 3.38-3.35 (m, 5H), 3.08 (t, J=4.9 Hz, 8H), 2.98 (d, J=10.2 Hz, 2H), 2.92 (s, 2H), 2.88 (s, 3H), 2.80 (s, 3H), 2.47 (s, 7H), 2.39 (s, 2H), 2.41-2.18 (m, 5H), 2.02-1.98 (m, 3H), 1.55 (s, 5H), 0.82 (d, J=6.9 Hz, 6H). LC-MS: m/z 1321.4 [M+H]⁺.

### Compound 328

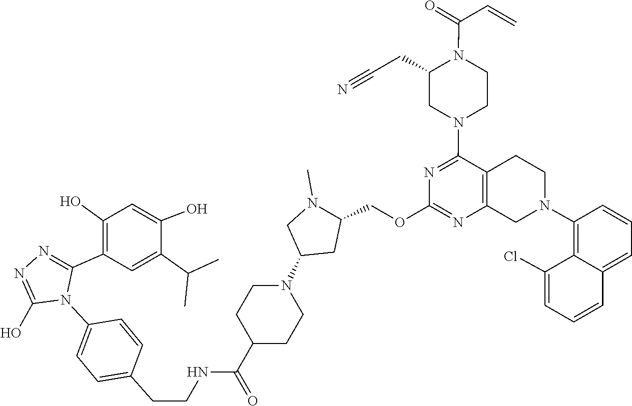 [SMILES: C=CC(=O)N1CCN(c2nc(OC[C@@H]3C[C@H](N4CCC(C(=O)NCCc5ccc(-n6c(O)nnc6-c6cc(C(C)C)c(O)cc6O)cc5)CC4)CN3C)nc3c2CCN(c2cccc4cccc(Cl)c24)C3)C[C@@H]1CC#N]

### 1-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methyl pyrrolidin-3-yl]-N-(2-{4-[3-(2,4-dihydroxy-5-isopropylphenyl)-5-hydroxy-1,2,4-triazol-4-yl]phenyl}ethyl)piperidine-4-carboxamide

¹H NMR (DMSO-d₆, 400 MHz): δ 11.89 (s, 1H), 9.57 (s, 1H), 9.38 (s, 1H), 7.96-7.89 (m, 1H), 7.81 (t, J=5.5 Hz, 1H), 7.75 (dd, J=8.1, 4.2 Hz, 1H), 7.59 (d, J=7.6 Hz, 1H), 7.52 (d, J=7.6 Hz, 1H), 7.45 (t, J=8.0 Hz, 1H), 7.35 (dd, J=17.1, 7.5 Hz, 1H), 7.19 (d, J=8.0 Hz, 2H), 7.09 (d, J=8.0 Hz, 2H), 6.80 (s, 2H), 6.25 (s, 1H), 6.19 (d, J=16.6 Hz, 1H), 5.77 (d, J=11.3 Hz, 1H), 4.97-4.77 (m, 1H), 4.29 (dd, J=10.8, 4.9 Hz, 2H), 4.19-4.11 (m, 2H), 4.04-4.00 (m, 2H), 3.73 (s, 2H), 3.51 (m, 4H), 3.23 (s, 2H), 3.17-3.04 (m, 4H), 3.01-2.83 (m, 3H), 2.81 (m, 5H), 2.27 (d, J=3.6 Hz, 3H), 2.02 s, 2H), 1.88 (s, 2H), 1.58 (s, 6H), 0.97 (d, J=6.9 Hz, 6H). LC-MS: m/z 1049.3 [M+H]⁺.

### Compound 329

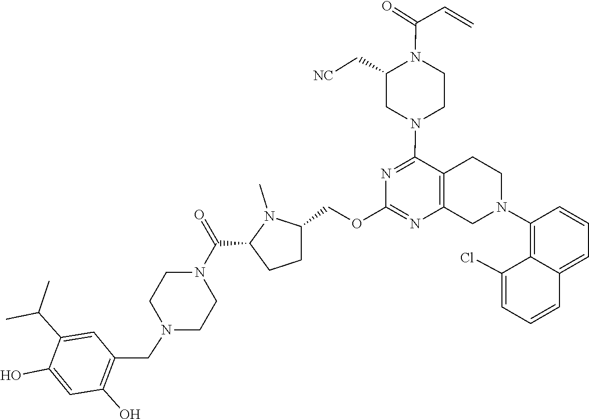 [SMILES: C=CC(=O)N1CCN(c2nc(OC[C@@H]3CC[C@H](C(=O)N4CCN(Cc5cc(C(C)C)c(O)cc5O)CC4)N3C)nc3c2CCN(c2cccc4cccc(Cl)c24)C3)C[C@@H]1CC#N]

### 2-[(2S)-4[7-(8chloronaphthalen-1-yl)-2-{[(2S,5R)-5-{4-[(2,4-dihydroxy-5-isopropylphenyl)methyl]piperazine-1-carbonyl}-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 9.66 (s, 1H), 9.01 (s, 1H), 7.93 (d, J=8.1 Hz, 1H), 7.75 (dd, J=8.1, 4.8 Hz, 1H), 7.60-7.51 (m, 2H), 7.47-7.43 (m, 1H), 7.38-7.31 (m, 1H), 6.84 (s, 1H), 6.75 (s, 1H), 6.23 (s, 1H), 6.19 (d, J=16.8 Hz, 1H), 5.78 (d, J=10.5 Hz, 1H), 4.96-4.77 (m, 1H), 4.39-4.22 (m, 2H), 4.19-3.94 (m, 5H), 3.82-3.49 (m, 6H), 3.46 (s, 2H), 3.12-2.91 (m, 8H), 2.69-2.67 (m, 1H), 2.40 (s, 2H), 2.33-2.27 (m, 5H), 1.97 (s, 2H), 1.67 (s, 2H), 1.09 (d, J=6.9 Hz, 6H). LC-MS: m/z 862.3 [M+H]⁺.

### Compound 330

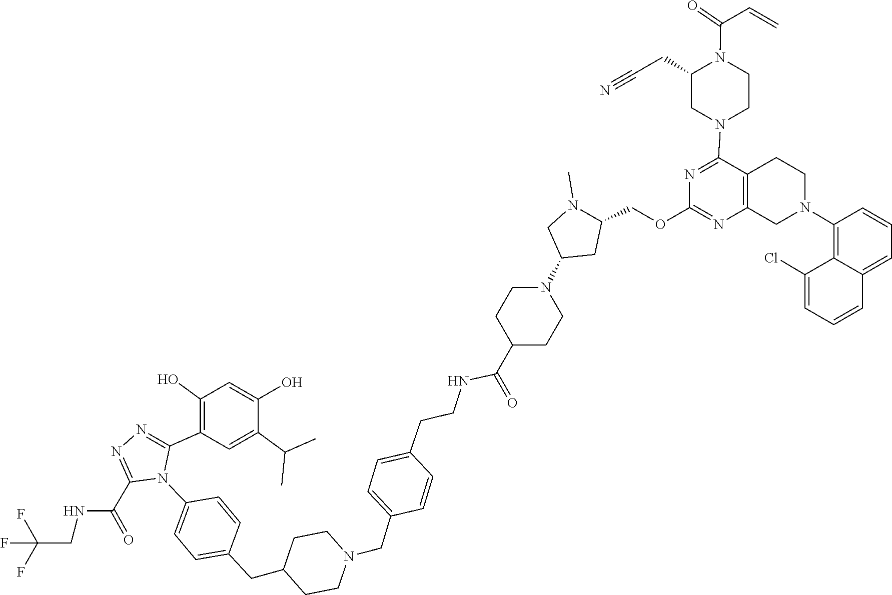 [SMILES: C=CC(=O)N1CCN(c2nc(OC[C@@H]3C[C@H](N4CCC(C(=O)NCCc5ccc(CN6CCC(Cc7ccc(-n8c(C(=O)NCC(F)(F)F)nnc8-c8cc(C(C)C)c(O)cc8O)cc7)CC6)cc5)CC4)CN3C)nc3c2CCN(c2cccc4cccc(Cl)c24)C3)C[C@@H]1CC#N]

### 1-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methyl pyrrolidin-3-yl]-N-[2-(4-{[4-({4-[3-(2,4-dihydroxy-5-isopropylphenyl)-5-[(2,2,2-tri fluoroethyl)carbamoyl]-1,2,4-triazol-4-yl]phenyl}methyl)piperidin-1-yl]methyl}phenyl)ethyl]piperidine-4-carboxamide

¹H NMR (DMSO-d₆, 400 MHz): δ 10.55 (s, 1H), 9.77 (s, 1H), 9.58 (t, J=6.6 Hz, 1H), 7.92 (d, J=8.2 Hz, 1H), 7.78-7.71 (m, 2H), 7.61-7.48 (m, 2H), 7.45 (t, J=7.8 Hz, 1H), 7.34 (dd, J=16.7, 7.5 Hz, 1H), 7.30-7.20 (m, 4H), 7.18 (d, J=7.8 Hz, 2H), 7.11 (d, J=7.8 Hz, 2H), 6.85 (s, 1H), 6.60 (s, 1H), 6.35 (s, 1H), 6.19 (d, J=16.6 Hz, 1H), 5.77 (d, J=10.9 Hz, 1H), 4.97-4.77 (m, 1H), 4.43-4.09 (m, 4H), 4.06-3.89 (m, 4H), 3.77-3.45 (m, 1H), 3.37 (s, 2H), 3.23 (s, 3H), 3.17-2.99 (m, 4H), 2.96 (s, 1H), 2.95-2.86 (m, 4H), 2.77-2.75 (m, 3H), 2.66 (d, J=7.6 Hz, 3H), 2.54 (s, 2H), 2.34-2.24 (m, 4H), 2.05-1.97 (m, 2H), 1.95-1.76 (m, 4H), 1.55-1.43 (m, 8H), 1.26-1.17 (m, 3H), 0.80 (d, J=6.9 Hz, 6H). LC-MS: m/z 1345.4 [M+H]⁺.

### Compound 331

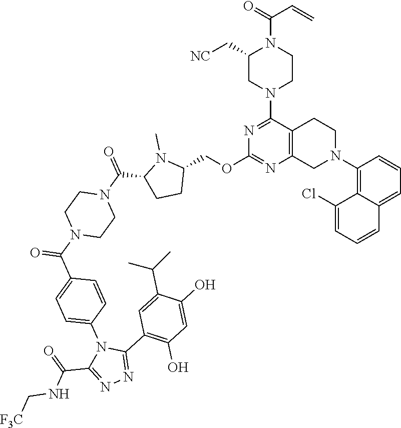 [SMILES: C=CC(=O)N1CCN(c2nc(OC[C@@H]3CC[C@H](C(=O)N4CCN(C(=O)c5ccc(-n6c(C(=O)NCC(F)(F)F)nnc6-c6cc(C(C)C)c(O)cc6O)cc5)CC4)N3C)nc3c2CCN(c2cccc4cccc(Cl)c24)C3)C[C@@H]1CC#N]

### 4-(4-{4-[(2R,5S)-5-({[7-(8-chloronaphthalen-1-yl)-4-[(3S)-3-(cyanomethyl)-4-(pro p-2-enoyl)piperazin-1-yl]-5H,6H,8H-pyrido[3,4-d]pyrimidin-2-yl]oxy}methyl)-1-methylpyrrolidine-2-carbonyl]piperazine-1-carbonyl}phenyl)-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.15 (s, 1H), 9.72 (s, 1H), 9.64 (t, J=6.5 Hz, 1H), 7.92 (d, J=7.9 Hz, 1H), 7.74 (dd, J=8.0, 5.0 Hz, 1H), 7.58-7.32 (m, 7H), 6.85 (s, 1H), 6.72 (s, 1H), 6.33 (s, 1H), 6.18 (d, J=16.0 Hz, 1H), 5.76 (d, J=10.5 Hz, 1H), 4.96-4.76 (m, 1H), 4.39-4.13 (m, 3H), 3.99-3.95 (m, 6H), 3.74-3.63 (m, 4H), 3.49 (s, 2H), 3.22 (s, 2H), 3.21-3.08 (m, 5H), 2.97-2.92 (m, 2H), 2.70-2.66 (m, 1H), 2.50 (s, 4H), 2.30-2.29 (m, 3H), 2.08-1.99 (m, 2H), 1.73 (s, 2H), 0.89 (d, J=6.8 Hz, 6H). LC-MS: m/z 1144.2 [M+H]⁺.

### Compound 332

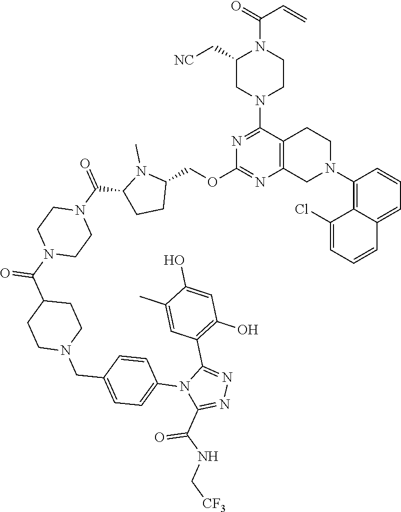 [SMILES: C=CC(=O)N1CCN(c2nc(OC[C@@H]3CC[C@H](C(=O)N4CCN(C(=O)C5CCN(Cc6ccc(-n7c(C(=O)NCC(F)(F)F)nnc7-c7cc(C)c(O)cc7O)cc6)CC5)CC4)N3C)nc3c2CCN(c2cccc4cccc(Cl)c24)C3)C[C@@H]1CC#N]

### 4-{4-[(4-{4-[(2R,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidine-2-carbonyl]piperazine-1-carbonyl}piperidin-1-yl)methyl]phenyl}-5-(2,4-dihydroxy-5-methylphenyl)-N-(2,2,2-trifluoroethyl)-1,2,4-triazole-3-carboxamide

¹H NMR (DMSO-d₆, 400 MHz): δ 10.06 (s, 1H), 9.68 (s, 1H), 9.60 (t, J=6.5 Hz, 1H), 7.91 (d, J=8.0 Hz, 1H), 7.73 (s, 1H), 7.61-7.59 (m, 2H), 7.45 (t, J=7.8 Hz, 1H), 7.38-7.23 (m, 5H), 6.85 (s, 1H), 6.64 (s, 1H), 6.30 (s, 1H), 6.19 (d, J=16.5 Hz, 1H), 5.77 (d, J=10.6 Hz, 1H), 4.97-4.78 (m, 1H), 4.24-4.15 (m, 4H), 4.01-3.92 (m, 4H), 3.81-3.77 (m, 2H), 3.53-3.48 (d, 7H), 3.42-3.39 (m, 4H), 3.30 (s, 3H), 3.10-3.07 (m, 5H), 2.78-2.66 (m, 4H), 2.29 (s, 3H), 2.00 (s, 4H), 1.84 (s, 3H), 1.72 (s, 2H), 1.57 (s 4H). LC-MS: m/z 1213.3 [M+H]⁺.

### Compound 333

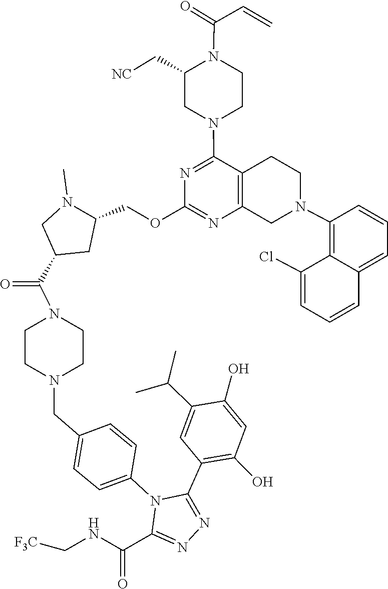 [SMILES: C=CC(=O)N1CCN(c2nc(OC[C@@H]3C[C@H](C(=O)N4CCN(Cc5ccc(-n6c(C(=O)NCC(F)(F)F)nnc6-c6cc(C(C)C)c(O)cc6O)cc5)CC4)CN3C)nc3c2CCN(c2cccc4cccc(Cl)c24)C3)C[C@@H]1CC#N]

### 4-[4-({4-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidine-3-carbonyl]piperazin-1-yl}methyl)phenyl]-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.41 (s, 1H), 9.75 (s, 1H), 9.59 (t, J=6.5 Hz, 1H), 7.96-7.86 (m, 1H), 7.75 (dd, J=7.9, 4.2 Hz, 1H), 7.63-7.49 (m, 2H), 7.48-7.41 (m, 1H), 7.39-7.28 (m, 5H), 6.85 (s, 1H), 6.64 (s, 1H), 6.34 (s, 1H), 6.19 (d, J=16.6 Hz, 1H), 5.78 (d, J=11.1 Hz, 1H), 4.96-4.77 (m, 1H), 4.52-4.08 (m, 4H), 4.05-3.91 (m, 4H), 3.90-3.62 (m, 2H), 3.55-3.41 (m, 7H), 3.26-2.98 (m, 2H), 2.96-2.83 (m, 2H), 2.68 (m, 3H), 2.66-2.54 (m, 1H), 2.38 (s, 3H), 2.35-2.33 (m, 2H), 2.31-2.29 (m, 4H), 2.18-2.09 (m, 1H), 1.89 (d, J=10.6 Hz, 1H), 0.84 (d, J=6.9 Hz, 6H). LC-MS: m/z 1130.2 [M+H]⁺.

### Compound 334

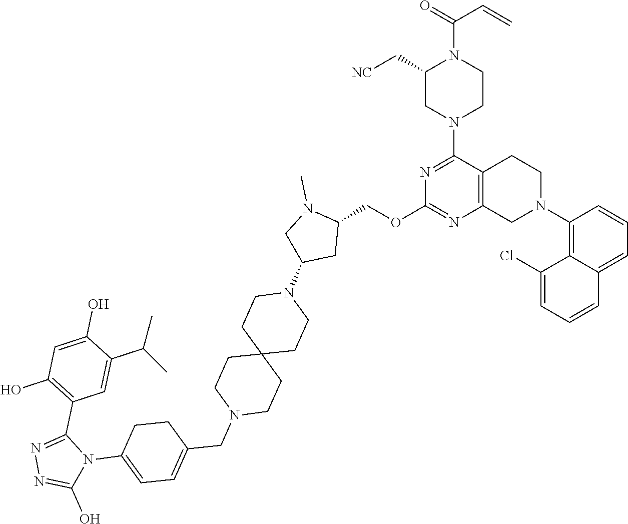 [SMILES: C=CC(=O)N1CCN(c2nc(OC[C@@H]3C[C@H](N4CCC5(CCN(CC6=CC=C(n7c(O)nnc7-c7cc(C(C)C)c(O)cc7O)CC6)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-[9-({4-[3-(2,4-dihydroxy-5-isopropylphenyl)-5-hydroxy-1,2,4-triazol-4-yl]phenyl}methyl)-3,9-diazaspiro[5.5]undecan-3-yl]-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 9.59 (s, 1H), 9.41 (s, 1H), 7.92 (dd, J=8.3, 1.3 Hz, 1H), 7.75 (dd, J=8.0, 3.9 Hz, 1H), 7.60-7.51 (m, 2H), 7.45 (t, J=7.8 Hz, 1H), 7.37 (d, J=7.5 Hz, 1H), 7.27 (d, J=8.1 Hz, 2H), 7.11 (d, J=8.3 Hz, 2H), 6.85 (s, 1H), 6.76 (s, 1H), 6.27 (s, 1H), 6.19 (d, J=16.7 Hz, 1H), 5.78 (d, J=10.5 Hz, 1H), 4.96-4.78 (m, 1H), 4.41-4.14 (m, 4H), 4.03-3.94 (m, 3H), 3.80-3.70 (m, 1H), 3.49 (s, 2H), 3.39 (s, 3H), 3.16-3.05 (m, 3H), 3.00-2.94 (m, 3H), 2.87-2.85 (m, 2H), 2.34 (p, J=1.9 Hz, 9H), 2.03-1.99 (m, 2H), 1.52-1.45 (m, 2H), 1.37 (s, 6H), 1.24 (s, 4H), 0.94 (d, J=6.9 Hz, 6H). LC-MS: m/z 1061.2 [M+H]⁺.

### Compound 335

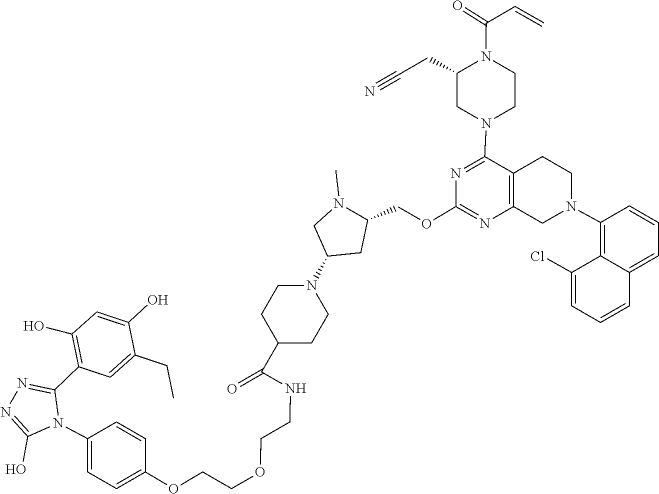 [SMILES: C=CC(=O)N1CCN(c2nc(OC[C@@H]3C[C@H](N4CCC(C(=O)NCCOCCOc5ccc(-n6c(O)nnc6-c6cc(CC)c(O)cc6O)cc5)CC4)CN3C)nc3c2CCN(c2cccc4cccc(Cl)c24)C3)C[C@@H]1CC#N]

### 1-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methyl pyrrolidin-3-yl]-N-[2-(2-{4-[3-(5-ethyl-2,4-dihydroxyphenyl)-5-hydroxy-1,2,4-tri-azol-4-yl]phenoxy}ethoxy)ethyl]piperidine-4-carboxamide

¹H NMR (DMSO-d₆, 400 MHz): δ 11.82 (s, 1H), 9.54 (s, 1H), 9.34 (s, 1H), 7.92 (d, J=8.0 Hz, 1H), 7.80-7.71 (m, 2H), 7.58-7.54 (m, 2H), 7.45 (t, J=7.8 Hz, 1H), 7.37-7.31 (m, 1H), 7.07 (d, J=8.9 Hz, 2H), 6.95-6.80 (m, 4H), 6.25-6.15 (m, 2H), 5.78 (d, J=10.7 Hz, 1H), 4.97-4.77 (m, 1H), 4.43-4.10 (m, 4H), 4.09-3.92 (m, 4H), 3.88-3.65 (m, 4H), 3.50 (s, 1H), 3.44 (t, J=5.9 Hz, 2H), 3.19 (q, J=6.0 Hz, 3H), 3.12-2.93 (m, 5H), 2.92-2.78 (m, 4H), 2.54 (s, 2H), 2.41-2.31 (m, 2H), 2.27 (d, J=3.0 Hz, 4H), 2.06-1.99 (m, 2H), 1.90-1.87 (m, 2H), 1.56-1.48 (m, 5H), 1.00 (t, J=7.4 Hz, 3H). LC-MS: m/z 1095.2 [M+H]⁺.

### Compound 336

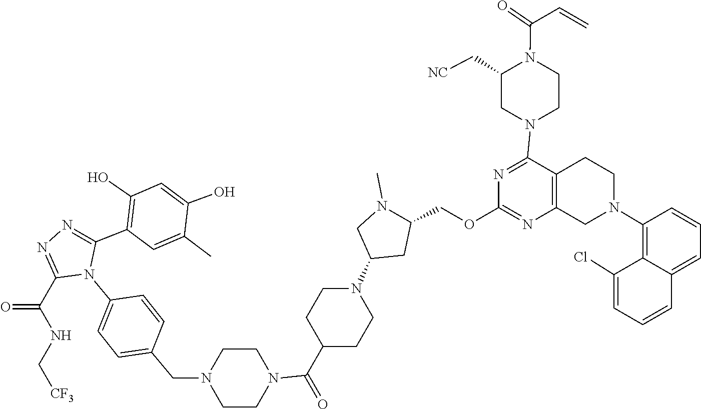 [SMILES: C=CC(=O)N1CCN(c2nc(OC[C@@H]3C[C@H](N4CCC(C(=O)N5CCN(Cc6ccc(-n7c(C(=O)NCC(F)(F)F)nnc7-c7cc(C)c(O)cc7O)cc6)CC5)CC4)CN3C)nc3c2CCN(c2cccc4cccc(Cl)c24)C3)C[C@@H]1CC#N]

### 4-{4-[(4-{1-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]piperidine-4-carbonyl}piperazin-1-yl)methyl]phenyl}-5-(2,4-dihydroxy-5-methylphenyl)-N-(2,2,2-trifluoroethyl)-1,2,4-triazole-3-carbox-amide

¹H NMR (DMSO-d₆, 400 MHz): δ 9.99 (s, 1H), 9.68 (s, 1H), 9.60 (t, J=6.5 Hz, 1H), 7.92 (d, J=8.0 Hz, 1H), 7.78-7.71 (m, 1H), 7.62-7.51 (m, 2H), 7.45 (t, J=7.8 Hz, 1H), 7.38-7.34 (m, 3H), 7.25 (d, J=8.4 Hz, 2H), 6.86 (s, 1H), 6.67 (s, 1H), 6.29 (s, 1H), 6.17 (d, J=16.0 Hz, 1H), 5.78 (d, J=11.0 Hz, 2H), 4.97-4.87 (m, 1H), 4.45-4.11 (m, 4H), 4.06-3.93 (m, 4H), 3.81-3.75 (m, 2H), 3.51 (s, 3H), 3.46 (s, 4H), 3.22-3.03 (m, 4H), 3.01-2.65 (m, 6H), 2.55 (d, J=5.9 Hz, 3H), 2.36 (s, 2H), 2.15 (s, 6H), 2.14-2.01 (m, 3H), 1.85 (s, 3H), 1.54 (s, 5H). LC-MS: m/z 1185.3 [M+H]⁺.

### Compound 337

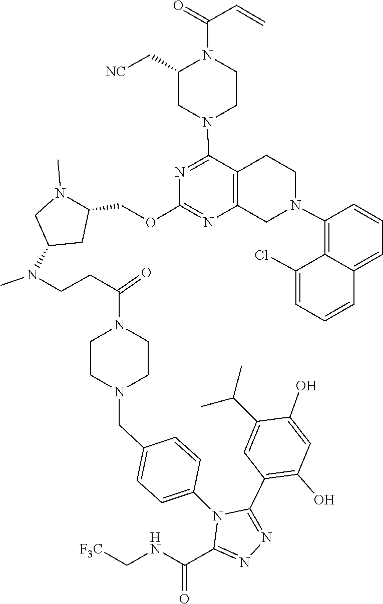 [SMILES: C=CC(=O)N1CCN(c2nc(OC[C@@H]3C[C@H](N(C)CCC(=O)N4CCN(Cc5ccc(-n6c(C(=O)NCC(F)(F)F)nnc6-c6cc(C(C)C)c(O)cc6O)cc5)CC4)CN3C)nc3c2CCN(c2cccc4cccc(Cl)c24)C3)C[C@@H]1CC#N]

### 4-(4-{[4-(3-{[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl](methyl) amino}propanoyl)piperazin-1-yl]methyl}phen-yl)-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.39 (s, 1H), 9.60 (s, 1H), 7.92 (d, J=8.1 Hz, 1H), 7.75 (dd, J=8.2, 4.3 Hz, 1H), 7.60-7.50 (m, 2H), 7.45 (t, J=7.8 Hz, 1H), 7.36-7.32 (m, 5H), 6.85 (d, J=8.5 Hz, 1H), 6.78 (s, 1H), 6.27 (s, 1H), 6.19 (d, J=16.7 Hz, 1H), 5.78 (d, J=10.7 Hz, 1H), 4.97-4.77 (m, 1H), 4.46-4.08 (m, 4H), 4.06-3.92 (m, 4H), 3.83-3.69 (m, 2H), 3.59 (s, 3H), 3.45 (s, 5H), 3.20-3.05 (m, 4H), 3.04-2.82 (m, 3H), 2.79 (s, 2H), 2.68 (s, 1H), 2.45-2.37 (m, 4H), 2.27 (d, J=3.8 Hz, 6H), 2.07 (s, 3H), 2.01 (s, 1H), 1.54 (s, 1H), 0.95 (d, J=6.9 Hz, 6H). LC-MS: m/z 1187.3 [M+H]⁺.

### Compound 338

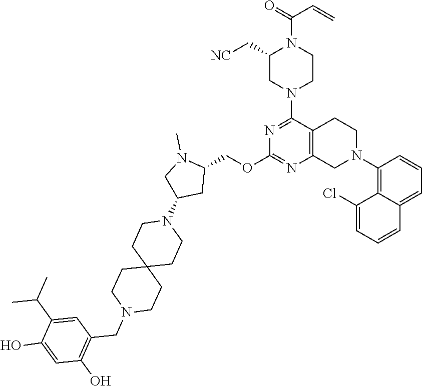 [SMILES: C=CC(=O)N1CCN(c2nc(OC[C@@H]3C[C@H](N4CCC5(CCN(Cc6cc(C(C)C)c(O)cc6O)CC5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{9-[(2,4-dihydroxy-5-isopropylphenyl)methyl]-3,9-diazaspiro[5.5]undecan-3-yl}-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 10.50 (s, 1H), 8.97 (s, 1H), 7.92 (d, J=8.1 Hz, 1H), 7.75 (dd, J=8.1, 4.3 Hz, 1H), 7.60-7.52 (m, 2H), 7.45 (t, J=7.8 Hz, 1H), 7.38-7.32 (m, 1H), 6.85 (s, 1H), 6.69 (d, J=2.6 Hz, 1H), 6.21-6.16 (m, 2H), 5.78 (d, J=10.6 Hz, 1H), 4.96-4.76 (m, 1H), 4.41-3.93 (m, 7H), 3.80-3.62 (m, 3H), 3.50 (s, 3H), 3.16-3.04 (m, 5H), 2.98-2.96 (m, 2H), 2.88-2.86 (m, 2H), 2.37 (s, 3H), 2.34-2.26 (m, 9H), 2.02 (d, J=4.7 Hz, 1H), 1.55-1.52 (m, 1H), 1.39 (s, 8H), 1.24 (s, 2H), 1.09 (d, J=6.9 Hz, 6H). LC-MS: m/z 902.3 [M+H]⁺.

### Compound 339

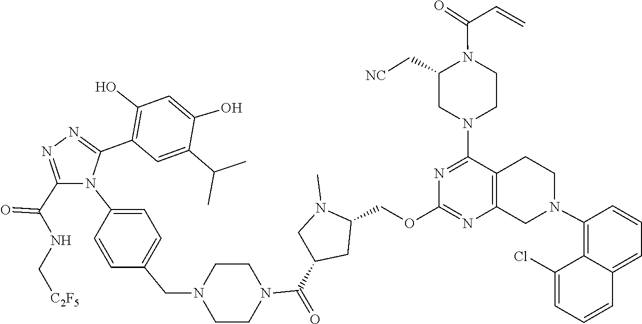

### 4-[4-({4-[(3S,5S)-5-({[7-(8-chloronaphthalen-1-yl)-4-[(3S)-3-(cyanomethyl)-4-(pr-op-2-enoyl)piperazin-1-yl]-5H,6H,8H-pyrido[3,4-d]pyrimidin-2-yl]oxy}methyl)-1-methylpyrrolidine-3-carbonyl]piperazin-1-yl}methyl)phenyl]-5-(2,4-dihydroxy-5-isopropylphenyl)-N-(2,2,3,3,3-pentafluoropropyl)-1,2,4-triazole-3-carboxamide

¹H NMR (DMSO-d₆, 400 MHz): δ 10.37 (s, 1H), 9.76 (s, 1H), 9.59 (t, J=6.5 Hz, 1H), 7.99-7.86 (m, 1H), 7.75 (dd, J=8.1, 4.3 Hz, 1H), 7.60-7.49 (m, 2H), 7.45 (t, J=7.8 Hz, 1H), 7.40-7.26 (m, 5H), 6.85 (s, 1H), 6.64 (s, 1H), 6.34 (s, 1H), 6.19 (d, J=16.4 Hz, 1H), 5.78 (dd, J=10.4, 2.3 Hz, 1H), 4.97-4.77 (m, 1H), 4.48-3.91 (m, 8H), 3.90-3.60 (m 2H), 3.52 (s, 3H), 3.46 (s, 4H), 3.20 (s, 3H), 3.15-3.02 (m, 4H), 2.99-2.83 (m, 3H), 2.76-2.56 (m, 2H), 2.41-2.36 (m, 2H), 2.35-2.24 (m, 5H), 2.14 (s, 1H), 1.87 (q, J=10.5, 9.3 Hz, 1H), 0.84 (d, J=6.9 Hz, 6H). LC-MS: m/z 1180.2 [M+H]⁺.

### Compound 340

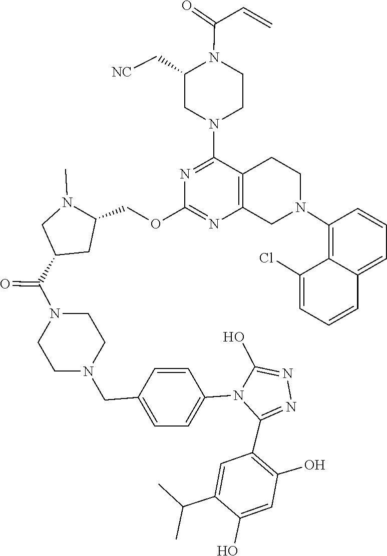 [SMILES: C=CC(=O)N1CCN(c2nc(OC[C@@H]3C[C@H](C(=O)N4CCN(Cc5ccc(-n6c(O)nnc6-c6cc(C(C)C)c(O)cc6O)cc5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-[4-({4-[3-(2,4-dihydroxy-5-isopropylphenyl)-5-hydroxy-1,2,4-triazol-4-yl]phenyl}methyl)piperazine-1-car-bonyl]-1-methylpyrrolidin-2-yl]methoxy}-5H,6H,8H-pyrido[3,4-d]pyrimidin-4-yl]-1-(prop-2-enoyl)piperazin-2-yl]acetonitrile

¹H NMR (DMSO-d₆, 400 MHz): δ 11.93 (s, 1H), 9.60 (s, 1H), 9.40 (s, 1H), 7.93 (d, J=8.1 Hz, 1H), 7.75 (dt, J=8.1, 2.7 Hz, 1H), 7.59 (dd, J=8.5, 2.1 Hz, 1H), 7.56-7.49 (m, 1H), 7.45 (t, J=7.8 Hz, 1H), 7.34 (dd, J=25.7, 8.0 Hz, 3H), 7.14 (d, J=8.2 Hz, 2H), 6.85 (s, 2H), 6.78 (s, 1H), 6.26 (s, 1H), 6.19 (d, J=16.4 Hz, 1H), 5.78 (d, J=10.5 Hz, 1H), 4.97-4.77 (m, 1H), 4.47-4.25 (m, 2H), 4.25-3.92 (m, 5H), 3.90-3.64 (m, 2H), 3.55-3.35 (m, 8H), 3.17 (s, 3H), 3.13-3.03 (m, 4H), 3.02-2.83 (m, 3H), 2.68 (p, J=1.9 Hz, 1H), 2.39-2.22 (m, 5H), 2.10 (d, J=17.0 Hz, 1H), 1.89 (d, J=18.1 Hz, 1H), 1.24 (s, 1H), 0.95 (d, J=6.9 Hz, 6H). LC-MS: m/z 1021.2 [M+H]⁺.

### Compound 341

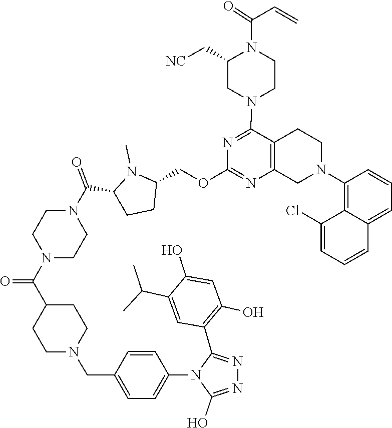 [SMILES: C=CC(=O)N1CCN(c2nc(OC[C@@H]3CC[C@H](C(=O)N4CCN(C(=O)C5CCN(Cc6ccc(-n7c(O)nnc7-c7cc(C(C)C)c(O)cc7O)cc6)CC5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2S,5R)-5-{4-[1-({4-[3-(2,4-dihydroxy-5-isopropylphenyl)-5-hydroxy-1,2,4-triazol-4-yl]phenyl}methyl)piperidine-4-carbonyl]piperazine-1-carbonyl}-1-methylpyrrolidin-2-yl]methoxy}-5H,6H,8H-pyri-do[3,4-d]pyrimidin-4-yl]-1-(prop-2-enoyl)piperazin-2-yl]acetonitrile

¹H NMR (DMSO-d₆, 400 MHz): δ 11.92 (s, 1H), 9.60 (s, 1H), 9.42 (s, 1H), 7.92 (d, J=8.1 Hz, 1H), 7.74 (t, J=6.3 Hz, 1H), 7.56 (dd, J=18.9, 7.7 Hz, 2H), 7.51-7.43 (m, 1H), 7.42-7.18 (m, 3H), 7.13 (d, J=7.9 Hz, 2H), 6.85 (s, 1H), 6.76 (s, 1H), 6.27 (s, 1H), 6.25-6.15 (m, 1H), 5.77 (d, J=10.5 Hz, 1H), 5.02-4.72 (m, 1H), 4.51-3.93 (m, 6H), 3.92-3.80 (m, 2H), 3.76-3.48 (m, 6H), 3.46-3.42 (m, 2H), 3.26 (s, 4H), 3.22-3.02 (m, 5H), 2.96 (p, J=6.7 Hz, 3H), 2.86-2.66 (m, 4H), 2.32-2.20 (m, 3H), 2.08-1.82 (m, 4H), 1.72 (s, 2H), 1.55 (s, 4H), 1.24 (s, 1H), 0.94 (d, J=6.8 Hz, 6H). LC-MS: m/z 1132.3 [M+H]⁺.

### Compound 342

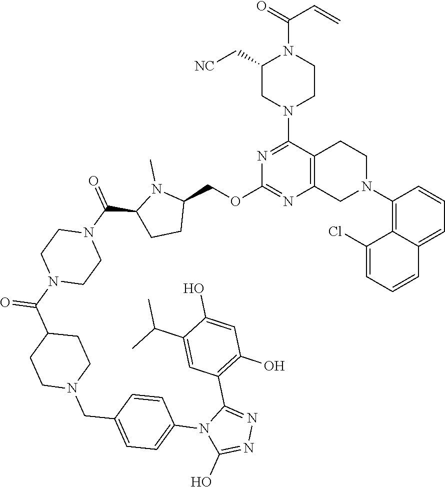 [SMILES: C=CC(=O)N1CCN(c2nc(OC[C@H]3CC[C@@H](C(=O)N4CCN(C(=O)C5CCN(Cc6ccc(-n7c(O)nnc7-c7cc(C(C)C)c(O)cc7O)cc6)CC5)CC4)N3C)nc3c2CCN(c2cccc4cccc(Cl)c24)C3)C[C@@H]1CC#N]

### 2-[(2S)-4-[7-(8-chloronaphthalen-1-yl)-2-{[(2R,5S)-5-{4-[1-({4-[3-(2,4-dihydroxy-5-isopropylphenyl)-5-hydroxy-1,2,4-triazol-4-yl]phenyl}methyl)piperidine-4-carbonyl]piperazine-1-carbonyl}-1-methylpyrrolidin-2-yl]methoxy}-5H,6H,8H-pyri-do[3,4-d]pyrimidin-4-yl]-1-(prop-2-enoyl)piperazin-2-yl]acetonitrile

¹H NMR (DMSO-d₆ 400 MHz): δ 11.93 (s, 1H), 9.60 (s, 1H), 9.42 (s, 1H), 7.92 (d, J=8.1 Hz, 1H), 7.78-7.70 (m, 1H), 7.64-7.52 (m, 2H), 7.51-7.41 (m, 1H), 7.40-7.19 (m, 3H), 7.16-7.08 (m, 2H), 6.85 (s, 1H), 6.76 (s, 1H), 6.27 (s, 1H), 6.24-6.13 (m, 1H), 5.84-5.70 (m, 1H), 5.02-4.82 (m, 1H), 4.53-3.93 (m, 6H), 3.83 (s, 2H), 3.68-3.40 (m, 7H), 3.30-3.26 (m, 4H), 3.20-2.89 (m, 7H) 2.88-2.65 (m, 4H), 2.48-2.44 (m, 2H), 2.38-2.22 (m, 3H), 2.04-1.80 (m, 4H), 1.72 (s, 2H), 1.56 (s, 4H), 0.94 (d, J=6.8 Hz, 6H). LC-MS: m/z 1132.2 [M+H]⁺.

### Compound 343

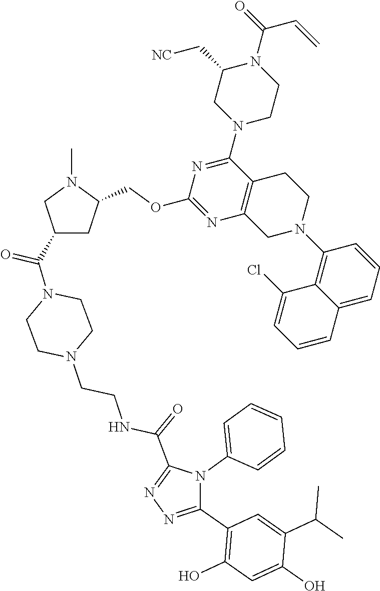 [SMILES: C=CC(=O)N1CCN(c2nc(OC[C@@H]3C[C@H](C(=O)N4CCN(CCNC(=O)c5nnc(-c6cc(C(C)C)c(O)cc6O)n5-c5ccccc5)CC4)CN3C)nc3c2CCN(c2cccc4cccc(Cl)c24)C3)C[C@@H]1CC#N]

### N-(2-{4-[(3S,5S)-5-({[7-(8-chloronaphthalen-1-yl)-4-[(3S)-3-(cyanomethyl)-4-(pr-op-2-enoyl)piperazin-1-yl]-5H,6H,8H-pyrido[3,4-d]pyrimidin-2-yl]oxy}methyl)-1-methylpyrrolidine-3-carbonyl]piperazin-1-yl}ethyl)-5-(2,4-dihydroxy-5-isopropylphenyl)-4-phenyl-1,2,4-triazole-3-carboxamide

¹H NMR (DMSO-d₆, 400 MHz): δ 10.39 (s, 1H), 9.72 (s, 1H), 8.82 (t, J=5.8 Hz, 1H), 7.93 (d, J=8.1 Hz, 1H), 7.79-7.71 (m, 1H), 7.62-7.50 (m, 2H), 7.49-7.40 (m, 4H), 7.39-7.29 (m, 3H), 6.85 (s, 1H), 6.60 (s, 1H), 6.32 (s, 1H), 6.19 (d, J=16.6 Hz, 1H), 5.78 (d, J=11.6 Hz, 1H), 4.98-4.78 (m, 1H), 4.46-3.91 (m, 6H), 3.90-3.55 (m, 2H), 3.54-3.36 (m, 5H), 3.28 (t, J=6.4 Hz, 2H), 3.18 (d, J=9.2 Hz, 2H), 3.14-2.99 (m, 4H), 2.98-2.85 (m, 2H), 2.74-2.55 (m, 2H), 2.42 (t, J=7.1 Hz, 4H), 2.36 (s, 3H), 2.29 (d, J=3.7 Hz, 4H), 2.17-2.09 (m, 1H), 1.87 (s, 1H), 0.84 (d, J=6.9 Hz, 6H). LC-MS: m/z 1062.2 [M+H]⁺.

### Compound 344

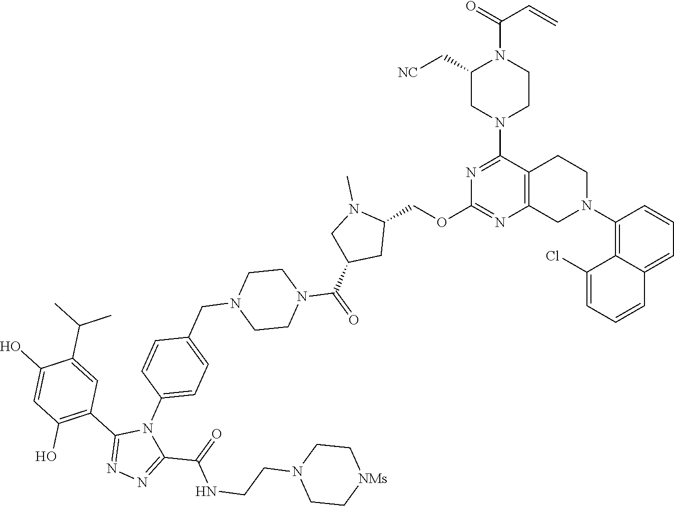

### 4-[4-({4-[(3S,5S)-5-({[7-(8-chloronaphthalen-1-yl)-4-[(3S)-3-(cyanomethyl)-4-(pr-op-2-enoyl)piperazin-1-yl]-5H,6H,8H-pyrido[3,4-d]pyrimidin-2-yl]oxy}methyl)-1-methylpyrrolidine-3-carbonyl]piperazin-1-yl}methyl)phenyl]-5-(2,4-dihydroxy-5-isopropylphenyl)-N-[2-(4-methanesulfonylpiperazin-1-yl)ethyl]-1,2,4-triazole-3-carboxamide

¹H NMR (DMSO-d₆, 400 MHz): δ 10.50 (s, 1H), 9.75 (s, 1H), 8.81 (t, J=5.9 Hz, 1H), 7.93 (dd, J=8.2, 1.3 Hz, 1H), 7.75 (dd, J=8.3, 4.4 Hz, 1H), 7.62-7.53 (m, 2H), 7.51-7.41 (m, 1H), 7.40-7.34 (m, 3H), 7.31 (d, J=8.2 Hz, 2H), 6.86 (s, 1H), 6.60 (s, 1H), 6.34 (s, 1H), 6.19 (d, J=16.5 Hz, 1H), 5.81-5.74 (m, 1H), 4.98-4.78 (m, 1H), 4.47-4.26 (m, 2H), 4.24-3.98 (m, 3H), 3.92-3.65 (m, 2H), 3.52 (s, 2H), 3.47 (s, 5H), 3.28 (t, J=6.3 Hz, 2H), 3.20 (s, 3H), 3.08 (s, 9H), 2.91 (t, J=6.7 Hz, 1H), 2.88 (s, 3H), 2.68 (s, 2H), 2.47 (t, J=6.4 Hz, 8H), 2.39 (s, 3H), 2.29 (d, J=3.9 Hz, 4H), 2.13 (s, 1H), 1.88 (d, J=8.7 Hz, 1H), 0.82 (d, J=6.9 Hz, 6H). LC-MS: m/z 1238.3 [M+H]⁺.

### Compound 345

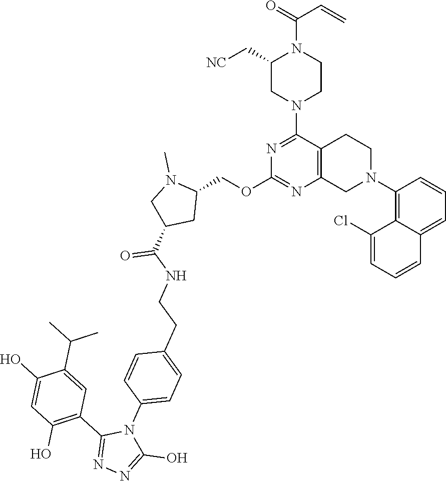 [SMILES: C=CC(=O)N1CCN(c2nc(OC[C@@H]3C[C@H](C(=O)NCCc4ccc(-n5c(O)nnc5-c5cc(C(C)C)c(O)cc5O)cc4)CN3C)nc3c2CCN(c2cccc4cccc(Cl)c24)C3)C[C@@H]1CC#N]

### (3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-N-(2-{4-[3-(2,4-dihydroxy-5-isopropylphenyl)-5-hydroxy-1,2,4-triazol-4-yl]phenyl}ethyl)-1-methylpyrrolidine-3-carboxamide

¹H NMR (DMSO-d₆, 400 MHz): δ 11.89 (s, 1H), 9.57 (s, 1H), 9.39 (d, J=1.6 Hz, 1H), 7.92 (dt, J=8.2, 2.1 Hz, 1H), 7.82 (t, J=5.6 Hz, 1H), 7.74 (dd, J=8.2, 4.4 Hz, 1H), 7.62-7.49 (m, 2H), 7.48-7.40 (m, 1H), 7.34 (dd, J=17.9, 7.4 Hz, 1H), 7.19 (d, J=8.4 Hz, 2H), 7.11-7.04 (m, 2H), 6.80 (d, J=8.4 Hz, 2H), 6.25 (s, 1H), 6.23-6.14 (m, 1H), 5.77 (dd, J=10.3, 2.3 Hz, 1H), 5.06-4.69 (m, 1H), 4.50-4.09 (m, 4H), 4.02 (d, J=14.6 Hz, 2H), 3.88-3.56 (m, 2H), 3.48 (d, J=12.2 Hz, 1H), 3.27-3.20 (m, 3H), 3.15-2.90 (m, 7H), 2.78-2.63 (m, 3H), 2.57 (dd, J=13.1, 7.1 Hz, 1H), 2.38 (t, J=8.9 Hz, 1H), 2.29 (dd, J=7.2, 3.8 Hz, 4H), 2.13 (dd, J=12.6, 8.2 Hz, 1H), 1.82 (dt, J=13.6, 7.4 Hz, 1H), 0.96 (d, J=6.9 Hz, 6H). LC-MS: m/z 966.2 [M+H]⁺.

### Compound 346

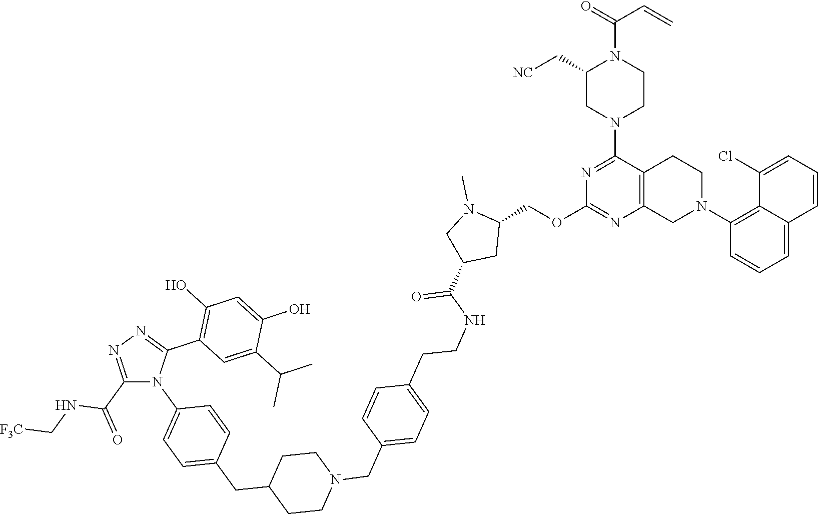 [SMILES: C=CC(=O)N1CCN(c2nc(OC[C@@H]3C[C@H](C(=O)NCCc4ccc(CN5CCC(Cc6ccc(-n7c(C(=O)NCC(F)(F)F)nnc7-c7cc(C(C)C)c(O)cc7O)cc6)CC5)cc4)CN3C)nc3c2CCN(c2cccc4cccc(Cl)c24)C3)C[C@@H]1CC#N]

### 4-{4-[(1-{[4-(2-{[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidin-3-yl]formamido}ethyl)phenyl]methyl}piperidin-4-yl)methyl]phenyl}-5-(2,4-dihydroxy-5-isopropylphenyl)-N-(2,2,2-trifluoroethyl)-1,2,4-triazole-3-carboxamide

¹H NMR (DMSO-d₆, 400 MHz): δ 10.54 (s, 1H), 9.77 (s, 1H), 9.58 (t, J=6.5 Hz, 1H), 7.92 (dd, J=8.3, 1.3 Hz, 1H), 7.80-7.69 (m, 2H), 7.62-7.48 (m, 2H), 7.44 (t, J=7.8 Hz, 1H), 7.40-7.17 (m, 6H), 7.13 (q, J=8.0 Hz, 4H), 6.85 (s, 1H), 6.60 (s, 1H), 6.35 (s, 1H), 6.19 (d, J=16.6 Hz, 1H), 5.77 (d, J=11.4 Hz, 1H), 4.86 (m, 1H), 4.50-4.22 (m, 2H), 4.18-3.85 (m, 7H), 3.83-3.72 (m, 2H), 3.67-3.41 (m, 2H), 3.28-3.24 (m, 2H), 3.06 (m, 6H), 2.95-2.82 (m, 3H), 2.75-2.70 (m, 4H), 2.44-2.30 (m, 1H), 2.29-2.26 (m, 3H), 2.20-2.04 (m, 3H), 1.85-1.72 (m, 3H), 1.61-1.38 (m, 4H), 1.30-1.09 (m, 3H), 0.84-0.78 (m, 6H). LC-MS: m/z 1262.3 [M+H]⁺.

### Compound 347

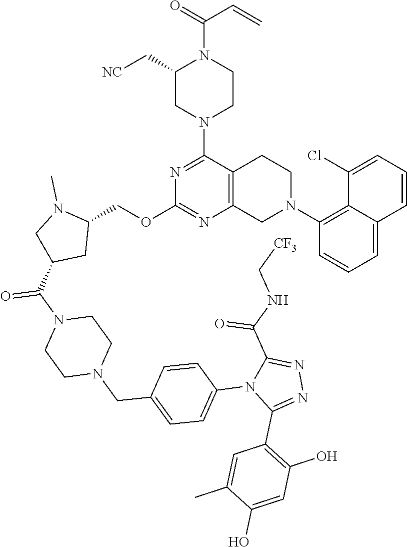 [SMILES: C=CC(=O)N1CCN(c2nc(OC[C@@H]3C[C@H](C(=O)N4CCN(Cc5ccc(-n6c(C(=O)NCC(F)(F)F)nnc6-c6cc(C)c(O)cc6O)cc5)CC4)CN3C)nc3c2CCN(c2cccc4cccc(Cl)c24)C3)C[C@@H]1CC#N]

### 4-[4-({4-[(3S,5S)-5-({[7-(8-chloronaphthalen-1-yl)-4-[(3S)-3-(cyanomethyl)-4-(prop-2-enoyl)piperazin-1-yl]-5H,6H,8H-pyrido[3,4-d]pyrimidin-2-yl]oxy}methyl)-1-methylpyrrolidine-3-carbonyl]piperazin-1-yl}methyl)phenyl]-5-(2,4-dihydroxy-5-methylphenyl)-N-(2,2,2-trifluoroethyl)-1,2,4-triazole-3-carboxamide

¹H NMR (DMSO-d₆, 400 MHz): δ 9.99 (s, 1H), 9.69 (s, 1H), 9.61 (t, J=6.6 Hz, 1H), 7.96-7.89 (m, 1H), 7.75 (dd, J=8.0, 4.3 Hz, 1H), 7.59 (d, J=7.4 Hz, 2H), 7.44 (q, J=8.0 Hz, 1H), 7.35 (t, J=7.8 Hz, 1H), 7.32 (dd, J=18.1, 7.7 Hz, 2H), 7.25 (d, J=8.0 Hz, 2H), 6.86 (s, 1H), 6.67 (s, 1H), 6.29 (s, 1H), 6.19 (d, J=16.6 Hz, 1H), 5.80-5.75 (m, 1H), 5.00-4.79 (m, 1H), 4.60-4.22 (m, 3H), 4.21-4.10 (m, 2H), 4.08-3.92 (m, 4H), 3.91-3.60 (m, 6.6 Hz, 3H), 3.55-3.50 m, 2H), 3.48-3.42 (m, 4H), 3.32 (s, 3H), 3.22-3.15 (m, 2H), 3.14-2.93 (m, 5H), 2.90-2.85 (m, 1H), 2.52-2.45 (m, 1H), 2.33-2.25 (m, 4H), 2.18-2.07 (s, 1H), 1.85 (s, 3H). LC-MS: m/z 1102.2 [M+H]⁺.

### Compound 348

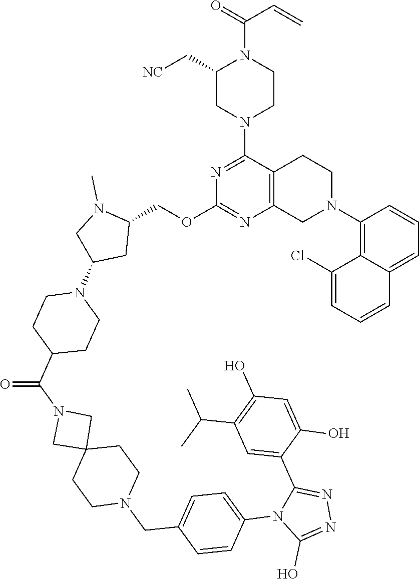 [SMILES: C=CC(=O)N1CCN(c2nc(OC[C@@H]3C[C@H](N4CCC(C(=O)N5CC6(CCN(Cc7ccc(-n8c(O)nnc8-c8cc(C(C)C)c(O)cc8O)cc7)CC6)C5)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{4-[7-({4-[3-(2,4-dihydroxy-5-isopropylphenyl)-5-hydroxy-1,2,4-triazol-4-yl]phenyl}methyl)-2,7-diazaspiro [3.5]nonane-2-carbonyl]piperidin-1-yl}-1-methylpyrrolidin-2-yl]methoxy}-5H,6-H,8H-pyrido[3,4-d]pyrimidin-4-yl]-1-(prop-2-enoyl)piperazin-2-yl]acetonitrile

¹H NMR (DMSO-d₆, 400 MHz): δ 11.94 (s, 1H), 9.61 (s, 1H), 9.42 (s, 1H), 7.93 (d, J=8.1 Hz, 1H), 7.78-7.71 (m, 1H), 7.63-7.50 (m, 2H), 7.45 (t, J=7.8 Hz, 1H), 7.37 (d, J=7.4 Hz, 1H), 7.28 (d, J=8.0 Hz, 2H), 7.13 (d, J=8.0 Hz, 2H), 6.77 (s, 2H), 6.27-6.17 (m, 2H), 5.78 (d, J=10.5 Hz, 1H), 5.01-4.96 (m, 1H), 4.29 (s, 2H), 4.21-4.07 (m, 2H), 4.03 (s, 2H), 3.88-3.72 (m, 4H), 3.48 (s, 4H), 3.19-3.05 (m, 6H), 3.01-2.91 (m, 4H), 2.34-2.25 (m, 4H), 1.64 (s, 6H), 1.50 (s, 4H), 1.24 (s, 4H), 1.15 (s, 6H), 0.94 (d, J=6.9 Hz, 6H), 0.85 (d, J=7.0 Hz, 1H). LC-MS: m/z 1144.4 [M+H]⁺.

### Compound 349

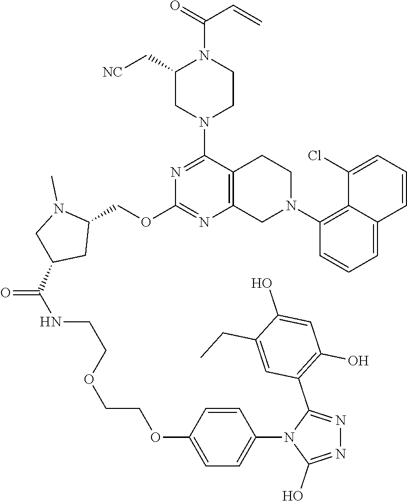 [SMILES: C=CC(=O)N1CCN(c2nc(OC[C@@H]3C[C@H](C(=O)NCCOCCOc4ccc(-n5c(O)nnc5-c5cc(CC)c(O)cc5O)cc4)CN3C)nc3c2CCN(c2cccc4cccc(Cl)c24)C3)C[C@@H]1CC#N]

### (3S,5S)-5-({[7-(8-chloronaphthalen-1-yl)-4-[(3S)-3-(cyanomethyl)-4-(prop-2-enoyl) piperazin-1-yl]-5H,6H,8H-pyrido[3,4-d]pyrimidin-2-yl]oxy}methyl)-N-[2-(2-{4-[3-(5-ethyl-2,4-dihydroxyphenyl)-5-hydroxy-1,2,4-triazol-4-yl]phenoxy}ethoxy)eth yl]-1-methylpyrrolidine-3-carboxamide

¹H NMR (DMSO-d₆, 400 MHz): δ 11.83 (s, 1H), 9.54 (s, 1H), 9.33 (s, 1H), 7.96-7.88 (m, 1H), 7.81 (t, J=5.5 Hz, 1H), 7.78-7.70 (m, 1H), 7.59-7.48 (m, 2H), 7.44 (t, J=7.8 Hz, 1H), 7.38-7.30 (m, 1H), 7.06 (d, J=8.7 Hz, 2H), 6.91-6.83 (m, 4H), 6.24 (s, 1H), 6.19 (d, J=16.6 Hz, 1H), 5.77 (d, J=10.8 Hz, 1H), 5.02-4.75 (m, 1H), 4.54-3.96 (m, 9H), 3.91-3.54 (m, 5H), 3.52-3.46 (m, 3H), 3.26-3.20 (m, 3H), 3.16-2.96 (m, 5H), 2.95-2.83 (m, 1H), 2.80-2.75 (m, 1H), 2.62 (d, J=1.8 Hz, 1H), 2.45-2.36 (m, 2H), 2.28 (d, J=3.8 Hz, 3H), 2.20-2.05 (m, 1H), 1.84-1.75 (s, 1H), 0.99 (t, J=7.5 Hz, 3H). LC-MS: m/z 1012.1 [M+H]⁺.

### Compound 350

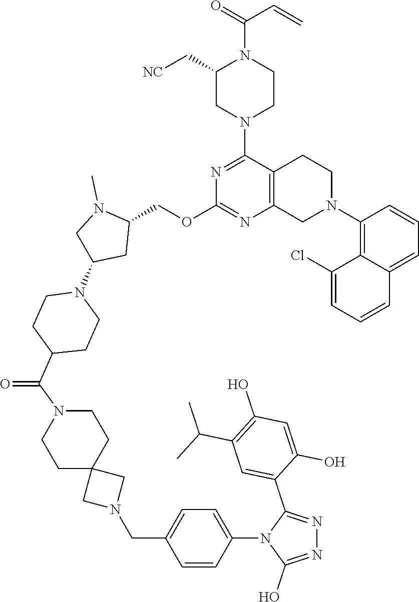 [SMILES: C=CC(=O)N1CCN(c2nc(OC[C@@H]3C[C@H](N4CCC(C(=O)N5CCC6(CC5)CN(Cc5ccc(-n7c(O)nnc7-c7cc(C(C)C)c(O)cc7O)cc5)C6)CC4)CN3C)nc3c2CCN(c2cccc4cccc(Cl)c24)C3)C[C@@H]1CC#N]

### 2-[(2S)-4-[7-(8-chloronaphthalen-1-yl)-2-{[(2S,4S)-4-{4-[2-({4-[3-(2,4-dihydroxy-5-isopropylphenyl)-5-hydroxy-1,2,4-triazol-4-yl]phenyl}methyl)-2,7-diazaspiro [3.5]nonane-7-carbonyl]piperidin-1-yl}-1-methylpyrrolidin-2-yl]methoxy}-5H,6-H,8H-pyrido[3,4-d]pyrimidin-4-yl]-1-(prop-2-enoyl)piperazin-2-yl]acetonitrile

¹H NMR (DMSO-d₆, 400 MHz): δ 11.91 (s, 1H), 9.58 (s, 1H), 9.40 (s, 1H), 7.92 (d, J=8.1 Hz, 1H), 7.75 (dd, J=8.2, 4.7 Hz, 1H), 7.61-7.50 (m, 2H), 7.45 (t, J=7.8 Hz, 1H), 7.38-7.31 (m, 1H), 7.26 (d, J=8.2 Hz, 2H), 7.12 (d, J=8.3 Hz, 2H), 6.84-6.77 (m, 2H), 6.26 (s, 1H), 6.21-6.16 (m, 1H), 5.79-5.76 (m, 1H), 4.95-4.76 (m, 1H), 4.56 (s, 1H), 4.40-4.11 (m, 4H), 4.08-3.91 (m, 2H), 3.84 (s, 1H), 3.76 (s, 1H), 3.74-3.42 (m, 5H), 3.20-3.02 (m, 5H), 3.01-2.85 (m, 9H), 2.81 (s, 3H), 2.35-2.25 (m, 4H), 2.14-1.87 (m, 4H), 1.73-1.45 (m, 10H), 0.96 (d, J=6.9 Hz, 6H). LC-MS: m/z 1144.2 [M+H]⁺.

### Compound 351

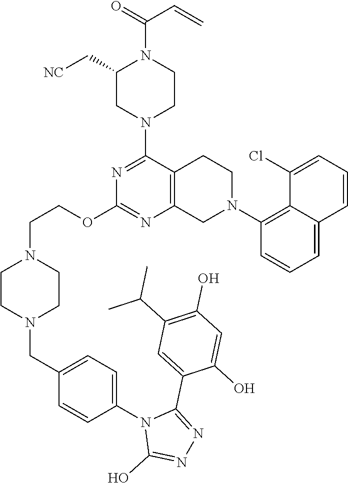 [SMILES: C=CC(=O)N1CCN(c2nc(OCCN3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N]

### 2-[(2S)-4-[7-(8-chloronaphthalen-1-yl)-2-{2-[4-({4-[3-(2,4-dihydroxy-5-isopropyl phenyl)-5-hydroxy-1,2,4-triazol-4-yl]phenyl}methyl)piperazin-1-yl]ethoxy}-5H,6-H,8H-pyrido[3,4-d]pyrimidin-4-yl]-1-(prop-2-enoyl)piperazin-2-yl]acetonitrile

¹H NMR (DMSO-d₆, 400 MHz): δ 11.92 (s, 1H), 9.58 (s, 1H), 9.41 (s, 1H), 7.92 (dd, J=8.3, 1.3 Hz, 1H), 7.75 (dd, J=8.0, 3.8 Hz, 1H), 7.63-7.51 (m, 2H), 7.45 (t, J=7.8 Hz, 1H), 7.41-7.26 (m, 3H), 7.13 (d, J=8.1 Hz, 2H), 6.85 (s, 1H), 6.76 (s, 1H), 6.27 (s, 1H), 6.21-6.16 (m, 1H), 5.78 (d, J=10.5 Hz, 1H), 4.96-4.76 (m, 1H), 4.54-4.13 (m, 4H), 4.11-3.92 (m, 2H), 3.91-3.59 (m, 2H), 3.58-3.46 (m, 1H), 3.43 (s, 2H), 3.25-3.02 (m, 4H), 3.01-2.81 (m, 3H), 2.68 (p, J=1.9 Hz, 3H), 2.64 (s, 4H), 2.36 (s, 2H), 2.34 (p, J=1.9 Hz, 2H), 0.93 (d, J=6.9 Hz, 6H). LC-MS: m/z 924.2 [M+H]⁺.

### Example 4: Testing Various CHAMP Molecules

Materials and Methods

Cell Lines

The following cancer cell lines were employed: BT-474 human breast carcinoma (ATCC, #HTB-20); MIA PaCa-2 human pancreatic carcinoma [2 copies KRAS(G12C)](ATCC, #CRL-1420); NCI-H23 human non-small cell lung cancer [1 copy KRAS(G12C)](ATCC, #CRL-5800); NCI-H358 human non-small cell lung cancer [1 copy KRAS(G12C)](ATCC, #CRL-5807); LU65A human lung carcinoma (Pharmaron, Beijing, China). Cell lines were cultured essentially according to ATCC and Pharmaron recommendations.

HSP90α-Binding Fluorescent Polarization (FP) Assay

Binding of test compounds to HSP90a protein was measured by fluorescent polarization (FP) using the HSP90a (N-terminal) Assay Kit (BPS Bioscience, #50298), following the manufacturer's instructions, except as noted. Fluorescently labeled HSP90-binding compounds, either the provided FITC-geldanamycin (5 nM final concentration) or RNK04010, a triazolone-based HSP90 binding small molecule labeled with BODIPY through a piperizine-phenyl linker (5 nM final concentration) were employed. A 2.5-fold serial dilution of each test compound ranging from 20 μM to 5.2 nM was assayed for binding to HSP90a. After the final step of adding HSP90a protein to each assay well, plates were mixed by brief shaking, incubated at 25° C. for 120 min for FITC-geldanamycin or 300 min for RNK04010, and fluorescence was measured using a PerkinElmer EnVision Plate Reader. Background-subtracted mP values were calculated from raw data and a four-parameter “log[inhibitor] vs. response” curve was fitted and IC50 values (the concentration at which 50% of the maximal inhibition occurs) calculated using GraphPad Prism 7 software.

KRAS(G12C)/SOS1 Homogeneous Time-Resolved Fluorescence (HTRF) Assay

Binding of test compounds to KRAS(G12C) protein, which in turn blocks KRAS(G12C) interaction with the SOS1 protein, was measured in the absences of GTP by homogeneous time-resolved fluorescence (HTRF) using the KRAS-G12C/SOS1 Binding Assay Kit (Cisbio, #63ADK000CB16PEG), following the manufacturer's instructions, except as noted. 3-fold serial dilutions of each test compound were prepared ranging from M to 1.02 nM. The test compound was mixed and incubated with reaction components, incubated in a sealed plate at 4° C. for 3 hr and fluorescence was measured using a PerkinElmer Envision plate reader. The % inhibition and IC50 values (the concentration at which 50% of the maximal inhibition occurs) were calculated and plotted using GraphPad Prism 7 software.

KRAS(G12C) Western Blot Protein Degradation Assay

The MIA PaCa-2 human pancreatic carcinoma cell line and LU65A human lung cancer cell line, which expresses KRAS(G12C), were seeded in 6- or 12-well tissue culture plates, and after 1 hr, test compounds were added at various concentrations and incubated at 37° C./5% CO₂ for 48 hr (MIA Paca-2) and 6 hr (LU65A) respectively. Cells were then washed with cold PBS, aspirated and cold RIPA buffer containing a protease/phosphatase inhibitor cocktail was added to lyse cells. After centrifugation, the total protein concentrations of cell lysates were determined using the BCA protein assay. Samples were normalized for equivalent protein concentrations, 5×SDS-PAGE loading buffer added and denatured at 100° C. for 10 min. 20 μl of each sample/well was loaded on a SDS-PAGE gel and electrophoresed for 20 min at 80 V, then 120 V for 1.5 hr. Gels were then electroblotted to nitrocellulose membranes using a wet-transfer method at 250 mA for 2.5 hr. Membranes were incubated with blocking buffer for 1 hr and washed 3 times with TBST for 5 min. Membranes were then incubated with anti-KRAS (Sigma-Aldrich, #SAB1404011) and anti-β-Actin monoclonal antibodies (Cell Signaling Technology, #3700) diluted in blocking buffer at 4° C. overnight per the manufacturer's recommendations. After washing 3 times, blots were incubated with appropriately labeled secondary antibodies for 1 hr at room temperature and washed again. Fluorescence imaging and quantitation was performed using a LI-COR Odyssey. Results was analyzed using GraphPad Prism 7 software. Compound degradation of was determined by the following equation and DC50 values (the concentration at which 50% of the maximal degradation of KRAS occurs) calculated using GraphPad Prism 7 software:

% Inhibition=100−(D−B)/(S−B)*100%.
S: Fluorescence intensity of cells with antibody
D: Fluorescence intensity of compound-treated cells with antibody
B: Fluorescence intensity of cells without antibody

ERBB2 (HER2) Flow Cytometry Protein Degradation Assay

BT-474 human breast carcinoma cells were plated in 24-well tissue culture plates at 250,000 cells/well and incubated at 37° C./5% CO₂ for 24 hr. Cells were then treated with test compounds at various concentrations and incubated at 37° C./5% CO₂ for 24 hr. To analyze changes in ERBB2 protein levels by flow cytometry, cells were detached with trypsin, washed, counted and treated with 10 μl/10⁶ cells PE-conjugated, anti-ERBB2 monoclonal antibody (R&D Systems, #FAB1129P) for 30 min at 25° C. in dark. Cells were then washed, resuspended in 200 μl 1% paraformaldehyde and analyzed by flow cytometry. Compound inhibition of was determined by the following equation and DC50 values (the concentration at which 50% of the maximal degradation of ERBB2 occurs) calculated using GraphPad Prism 7 software:

% Degradation=100−(D−B)/(S−B)*100%.
S: Fluorescence intensity of cells with antibody
D: Fluorescence intensity of compound-treated cells with antibody
B: Fluorescence intensity of cells without antibody

Cancer Cell Line Proliferation (CCK-8) Assays

Cells were plated in 96-well tissue culture plates at 4,000 cells/well and incubated at 37° C./5% CO₂ for 24 hr. 3-fold serial dilutions of each test compound were prepared ranging from 20 μM to 1.02 nM. Cells were then treated with test compounds at various concentrations with a final concentration of 0.5% DMSO/well, and then incubated at 37° C./5% CO₂ for 72 hr. 10 l the cell proliferation detection reagent CCK-8 (Dojindo Molecular Technologies, #CK04) was added to each well and incubated at 37° C./5% CO₂ for 3-4 hr and the absorbance at 450 nm measured using an Perkin Elmer EnVision Plate Reader. Compound inhibition of was determined by the following equation and EC50 values (the concentration at which 50% of the maximal inhibition occurs) calculated using GraphPad Prism 7 software:

% Inhibition=100−(D−B)/(S−B)*100%.
S: Absorbance of cells treated with DMSO
D: Absorbance of compound-treated cells
B: Absorbance of medium with DMSO without cells

Results

A number of synthetic schemes have been developed to construct various CHAMP molecules designed to degrade KRAS(G12C), which are termed KRAS(G12C)-CHAMP molecules. Representative examples are shown, each consisting of a HSP90 binder linked to a KRAS(G12C) binder. Similar chemistry can be applied to other CHAMP molecules not limited to these specific HSP90- and KRAS(G12C)-binding moieties.

HSP90α-binding fluorescent polarization (FP) assays measuring competition with the fluorescently labeled HSP90 binders, FITC-geldanamycin or RNK04010 (BODIPY-labeled), were applied to assess the binding capabilities of CHAMP molecules to HSP90. As shown in Table 1, CHAMP molecules containing HSP90-binding moieties documented in the literature were generally in agreement with the published structure activity relationship (SAR).

The incorporation of a KRAS(G12C) binder of similar molecular weight to the HSP90 binder into the CHAMPs typically had only minimal impact on the binding of CHAMP molecules to HSP90a in this assay (Table 1). There are a number of reasons: first the co-crystal structures of these moieties with their corresponding proteins are available and allow precise structure-based molecular designs; and secondly, the linker is constructed to provide rigidity with suitable length.

Binding by a variety of CHAMP molecules to KRAS(G12C) was assessed by measuring inhibition of KRAS(G12C) interaction with SOS1 in a HTRF biochemical assay, as shown in Table 1. CHAMP molecules containing KRAS(G12C)-binding moieties documented in the literature were generally in agreement with the published SAR.

The incorporation of a chaperone-binding moiety, such as a HSP90 binder, typically had only minimal impact on the binding of CHAMP molecules to KRAS(G12C) as measured by this assay. There are a number of reasons: first the co-crystal structures of these moieties with their corresponding proteins are available and allow precise structure-based molecular designs; and secondly, the linker is constructed to provide rigidity with suitable length.

Heterobifunctional CHAMP molecules with both a KRAS(G12C)-binding moiety and a HSP90-binding moiety are designed to induce targeted protein degradation (TPD) of KRAS(G12C). As shown in Table 2, MIA PaCa-2 human pancreatic carcinoma cells, which express KRAS(G12C), were treated for 48 hr with various concentrations of CHAMP compounds and degradation of KRAS(G12C) was observed by Western blotting.

CHAMP molecules may include chaperone or chaperone complex binders that have a range of different binding affinities. In different embodiments, it is desirable to use a high-affinity binder, a moderate-affinity binder or a low-affinity binder. Since a HSP9C-binding moiety that interacts with the N-terminal ATP-binding pocket of HSP9 may inhibit HSP90 activity and induce the degradation of HSP90 client proteins, some CHAMP molecules may not only induce the degradation of the desired target protein or proteins (which may or may not be HSP90 client proteins), but also simultaneously induce the degradation of HSP90 client proteins. As shown in Table 2, CHAMP compounds also displayed various levels of degradation of HSP90 client protein ERB1B2 as assessed by flow cytometry in ERB1B2-expressing BT-474 human breast carcinoma cells.

As shown in Table 1, various KRAS (G12C)-CHAMP molecules also inhibited the growth and/or survival of a panel of cancer cell lines as measured by a CCK-8 cell line proliferation assay.

TABLE 1
Biochemical and Cell-based Assays of Compounds

| | HSP90a | HSP90a | KRAS | | | |
|---|---|---|---|---|---|---|
| Compound | binding | binding | (G12C) | MIA PaCa-2 | NCI-H23 | NCI-H358 |
| # | (BODIPY)¹ | (FITC)² | binding³ | CCK-8⁴ | CCK-8⁵ | CCK-8⁶ |
| 1 | B | | | | | C |
| 2 | B | | | | | C |
| 3 | B | | | | | C |
| 4 | B | | | | | C |
| 5 | C | | | | | C |
| 6 | C | | | | | C |
| 7 | B | | | | | C |
| 8 | B | | | | | B |
| 9 | B | | | | | C |
| 10 | B | | | | B | B |
| 11 | C | | | | C | B |
| 12 | B | | | | | C |
| 13 | C | | | | | C |
| 14 | A | | C | C | C | B |
| 15 | A | | | | B | B |
| 16 | C | | C | A | B | B |
| 17 | B | | C | | B | B |
| 18 | B | | | | B | B |
| 19 | B | | | | B | B |
| 20 | C | | | | | C |
| 21 | A | | | B | B | B |
| 22 | A | | | | | C |
| 23 | B | | C | | B | B |
| 24 | B | | C | B | C | B |
| 25 | A | | | B | B | B |
| 26 | B | | | | | C |
| 27 | C | | | | | C |
| 28 | B | | | | | C |
| 29 | B | | | | | C |
| 30 | C | | | | | C |
| 31 | C | | | | | C |
| 32 | C | | | | | C |
| 33 | B | | | | | C |
| 34 | C | | | | | C |
| 35 | B | | | | | C |
| 36 | B | | | | | C |
| 37 | B | | | | | C |
| 38 | B | | | | | C |
| 39 | C | | | | | C |
| 40 | B | | | | | C |
| 41 | B | | | | | C |
| 42 | B | | | | | C |
| 43 | C | | | | | C |
| 44 | C | | | | | C |
| 45 | C | | | | | C |
| 46 | B | | | | | C |
| 47 | B | | | | | C |
| 48 | B | | | | | C |
| 49 | B | | | | | C |
| 50 | B | | | | | C |
| 51 | B | | | | | C |
| 52 | B | | | | | C |
| 53 | C | | | | | C |
| 54 | B | | C | A | B | B |
| 55 | A | | C | | B | B |
| 56 | B | | | | | C |
| 57 | B | | | | | C |
| 58 | B | | | | | C |
| 59 | B | | | | C | B |
| 60 | A | | | | C | B |
| 61 | A | | | | C | B |
| 62 | B | | C | | B | B |
| 63 | B | | | C | | B |
| 64 | B | | C | | B | B |
| 65 | B | | | | | C |
| 66 | C | | | | | C |
| 67 | B | | | | | C |
| 68 | C | | | | | C |
| 69 | A | | | | C | B |
| 70 | B | | | | | C |
| 71 | A | | | | C | B |
| 72 | A | | | | | C |
| 73 | B | | | | | C |
| 74 | B | | | | | C |
| 75 | A | | | | C | B |
| 76 | B | | C | | B | B |
| 77 | B | | | | | B |
| 78 | A | | | | B | B |
| 79 | B | | C | | B | B |
| 80 | B | | C | | | B |
| 81 | C | | | | | C |
| 82 | B | | | | | C |
| 83 | C | | | | | C |
| 84 | B | | | | | B |
| 85 | B | | | | | C |
| 86 | C | | | | | C |
| 87 | B | | | | B | B |
| 88 | B | | | | | C |
| 89 | C | | | | | C |
| 90 | B | | | | | C |
| 91 | C | | | | | C |
| 92 | A | | | | | C |
| 93 | B | | | | | C |
| 94 | C | | | | | C |
| 95 | B | | C | | B | B |
| 96 | B | | | | | C |
| 97 | B | | | | | C |
| 98 | A | | | | B | B |
| 99 | B | | | | B | B |
| 100 | B | | | | | C |
| 101 | A | | | | | C |
| 102 | A | | | | | C |
| 103 | A | | | | | C |
| 104 | A | | | | | C |
| 105 | C | | | | | C |
| 106 | C | | | | | C |
| 107 | C | | | | | C |
| 108 | B | | | | B | B |
| 109 | B | | | | B | B |
| 110 | C | | | C | | C |
| 111 | C | | | | | C |
| 112 | B | | | B | B | B |
| 113 | B | | | | | B |
| 114 | C | | | | | C |
| 115 | B | | | | B | B |
| 116 | A | | | B | B | B |
| 117 | B | | | | | B |
| 118 | C | | | | B | B |
| 119 | B | | | | B | B |
| 120 | B | | | B | B | A |
| 121 | A | | | B | B | A |
| 122 | C | | | | | C |
| 123 | C | | | B | | B |
| 124 | B | | | | | B |
| 125 | B | | | | | B |
| 126 | B | | | | | |
| 127 | B | | | | | C |
| 128 | B | | | | B | B |
| 129 | A | | | | | B |
| 130 | A | | | | | C |
| 131 | C | | | | B | C |
| 132 | B | | | | B | B |
| 133 | | B | | | | B |
| 134 | | B | | | | C |
| 135 | | B | | | | C |
| 136 | | C | | | | B |
| 137 | | B | | | | B |
| 138 | | C | | | | C |
| 139 | | B | C | C | | B |
| 140 | | B | | | | B |
| 141 | | B | | | | B |
| 142 | | C | | | | C |
| 143 | | C | | | | C |
| 144 | | B | | | | C |
| 145 | | B | | B | | B |
| 146 | | B | | | | B |
| 147 | | A | | B | | B |
| 148 | A | | B | B | | B |
| 149 | | B | | | | B |
| 150 | | A | | | | B |
| 151 | | B | | | | B |
| 152 | | A | | | | C |
| 153 | C | | | C | | C |
| 154 | B | | | | | B |
| 155 | B | | C | | | C |
| 156 | B | | C | | | B |
| 157 | C | | C | | | C |
| 158 | C | | C | C | | C |
| 159 | C | | C | | | C |
| 160 | A | | C | C | | C |
| 161 | B | | B | C | | C |
| 162 | C | | | | | C |
| 163 | C | | | | | C |
| 164 | C | | | | | C |
| 165 | | B | | C | | |
| 166 | | B | | A | | |
| 167 | | A | | B | | |
| 168 | | B | | A | | |
| 169 | | A | | B | | |
| 170 | | B | | C | | |
| 171 | | C | | C | | |
| 172 | C | B | | B | | |
| 173 | C | C | | C | | |
| 174 | C | C | | C | | |
| 175 | C | | | | | |
| 176 | C | | | | | |
| 177 | B | | | | | |
| 178 | C | | | C | | |
| 179 | A | | | B | | |
| 180 | B | | | C | | |
| 181 | C | | | C | | |
| 182 | A | | | C | | |
| 183 | B | | | B | | |
| 184 | B | | | B | | |
| 185 | B | | B | A | | |
| 186 | C | | B | | | |
| 187 | | | | | | |
| 188 | A | | C | B | | |
| 189 | A | | C | C | | |
| 190 | | | B | B | | |
| 191 | | | | | | |
| 192 | A | | A | B | | |
| 193 | A | | A | B | | |
| 194 | A | | B | B | | |
| 195 | A | | C | C | | |
| 196 | A | | B | C | | |
| 197 | A | | C | C | | |
| 198 | A | | B | B | | |
| 199 | A | | | B | | |
| 200 | A | | A | A | | |
| 201 | A | | B | C | | |
| 202 | A | | B | C | | |
| 203 | A | | B | C | | |
| 204 | A | | | C | | |
| 205 | A | | B | B | | |
| 206 | A | | B | B | | |
| 207 | A | | B | A | | |
| 208 | A | | C | A | | |
| 209 | A | | A | A | | |
| 210 | A | | A | A | | |
| 211 | A | | B | C | | |
| 212 | A | | B | B | | |
| 213 | A | | B | C | | |
| 214 | A | | A | B | | |
| 215 | A | | B | B | | |
| 216 | B | | A | B | | |
| 217 | B | | A | C | | |
| 218 | A | | A | C | | |
| 219 | A | | B | C | | |
| 220 | A | | B | B | | |
| 221 | A | | C | C | | |
| 222 | A | | B | B | | |
| 223 | A | | A | C | | |
| 224 | A | | C | B | | |
| 225 | A | | C | B | | |
| 226 | A | | | C | | |
| 227 | A | | A | B | | |
| 228 | A | | A | B | | |
| 229 | A | | A | C | | |
| 230 | A | | A | B | | |
| 231 | A | | B | C | | |
| 232 | A | | A | C | | |
| 233 | A | | A | C | | |
| 234 | A | | A | B | | |
| 235 | | | B | B | | |
| 236 | | | A | C | | |
| 237 | | | B | B | | |
| 238 | | | B | C | | |
| 239 | | | A | B | | |
| 240 | | | A | A | | |
| 241 | | | A | B | | |
| 242 | | | A | B | | |
| 243 | | | A | B | | |
| 244 | | | A | A | | |
| 245 | | | A | B | | |
| 246 | | | A | B | | |
| 247 | | | A | C | | |
| 248 | | | B | B | | |
| 249 | | | A | B | | |
| 250 | | | A | B | | |
| 251 | | | A | B | | |
| 252 | | | A | C | | |
| 253 | | | | B | | |
| 254 | | | | C | | |
| 255 | | | A | B | | |
| 256 | | | A | C | | |
| 257 | | | A | B | | |
| 258 | | | A | B | | |
| 259 | | | A | A | | |
| 260 | | | | A | | |
| 261 | | | A | A | | |
| 262 | | | A | B | | |
| 263 | | | A | A | | |
| 264 | | | A | B | | |
| 265 | | | A | C | | |
| 266 | | | B | C | | |
| 267 | | | A | C | | |
| 268 | | | A | B | | |
| 269 | | | A | A | | |
| 270 | | | | C | | |
| 271 | | | | B | | |
| 272 | | | | A | | |
| 273 | | | | B | | |
| 274 | | | A | B | | |
| 275 | | | A | B | | |
| 276 | | | A | A | | |
| 277 | | | A | B | | |
| 278 | | | A | B | | |
| 279 | | | A | B | | |
| 280 | | | A | A | | |
| 281 | | | A | B | | |
| 282 | | | A | B | | |
| 283 | | | A | B | | |
| 284 | | | A | B | | |
| 285 | | | A | A | | |
| 286 | | | B | A | | |
| 287 | | | B | A | | |
| 288 | | | A | A | | |
| 289 | | | A | A | | |
| 290 | | | A | A | | |
| 291 | | | | A | | |
| 292 | | | | B | | |
| 293 | A | | C | B | | |
| 294 | A | | C | C | | |
| 295 | A | | B | C | | |
| 296 | A | | B | C | | |
| 297 | A | | C | C | | |
| 298 | A | | C | B | | |
| 299 | A | | B | C | | |
| 300 | A | | A | A | | |
| 301 | A | | B | B | | |
| 302 | A | | B | B | | |
| 303 | A | | B | B | | |
| 304 | A | | B | A | | |
| 305 | A | | | B | | |
| 306 | A | | | C | | |
| 307 | A | | A | A | | |
| 308 | A | | A | A | | |
| 309 | A | | A | B | | |
| 310 | A | | A | A | | |
| 311 | A | | A | A | | |
| 312 | A | | A | C | | |
| 313 | A | | A | A | | |
| 314 | A | | A | A | | |
| 315 | A | | A | C | | |
| 316 | | | | | | |
| 317 | A | | A | B | | |
| 318 | A | | A | B | | |
| 319 | A | | A | B | | |
| 320 | A | | A | B | | |
| 321 | A | | A | B | | |
| 322 | | | | | | |
| 323 | A | | A | A | | |
| 324 | A | | B | B | | |
| 325 | A | | A | C | | |
| 326 | A | | A | B | | |
| 327 | A | | A | B | | |
| 328 | A | | A | C | | |
| 329 | A | | B | C | | |
| 330 | A | | B | B | | |
| 331 | A | | A | A | | |
| 332 | A | | A | A | | |
| 333 | A | | B | A | | |
| 334 | A | | A | A | | |
| 335 | A | | A | C | | |
| 336 | A | | A | B | | |
| 337 | A | | B | B | | |
| 338 | B | | A | C | | |
| 339 | A | | B | B | | |
| 340 | A | | A | B | | |
| 341 | A | | A | B | | |
| 342 | A | | A | B | | |
| 343 | A | | A | B | | |
| 344 | A | | A | B | | |
| 345 | A | | A | B | | |
| 346 | A | | A | B | | |
| 347 | A | | A | B | | |
| 348 | A | | A | B | | |
| 349 | A | | A | C | | |
| 350 | A | | A | B | | |
| 351 | A | | A | B | | |

¹HSP90α-binding FP (BODIPY) assay: A. IC50 < 100 nM; B. IC50 = 100-1000 nM; C. IC50 > 1000 nM;
²HSP90α-binding FP (FITC) assay: A. IC50 < 100 nM; B. IC50 = 100-1000 nM; C. IC50 > 1000 nM;
³KRAS(G12C)/SOS1 HTRF assay: A. IC50 < 100 nM; B. IC50 = 100-1000 nM; C. IC50 > 1000 nM;
⁴MIA PaCa-2 CCK-8 proliferation assay: A. EC50 < 100 nM; B. EC50 = 100-1000 nM; C. EC50 > 1000 nM;
⁵NCI-H23 CCK-8 proliferation assay: A. EC50 < 100 nM; B. EC50 = 100-1000 nM; C. EC50 > 1000 nM;
⁶NCI-H358 CCK-8 proliferation assay: A. EC50 < 100 nM; B. EC50 = 100-1000 nM; C. EC50 > 1000 nM

TABLE 2
ERBB2 and KRAS(G12C) Degradation by Compounds

| | | KRAS | KRAS | KRAS | KRAS | KRAS | KRAS | KRAS | |
|---|---|---|---|---|---|---|---|---|---|
| | | (G12C) | (G12C) | (G12C) | (G12C) | (G12C) | (G12C) | (G12C) | LU65A |
| | ERBB2 | degradation | degradation | degradation | degradation | degradation | degradation | degradation | at 6 H |
| Comp# | degradation¹ | (10,000nM)² | (5000 nM)² | (2500 nM)² | (1250 nM)² | (625 nM)² | (312.5 nM)² | (156.2 nM)² | DC50(nM)³ |
| 10 | C | | | | | | | | |
| 11 | C | | | | | | | | |
| 12 | C | | | | | | | | |
| 14 | B | | | | | | | | |
| 16 | C | C | C | C | C | C | C | C | |
| 17 | B | | | | | | | | |
| 18 | C | | | | | | | | |
| 19 | C | | | | | | | | |
| 21 | B | | | | | | | | |
| 23 | B | | | | | | | | |
| 24 | B | | | | | | | | |
| 25 | C | | | | | | | | |
| 53 | C | | | | | | | | |
| 54 | B | | | | | | | | |
| 55 | B | | | | | | | | |
| 58 | C | | | | | | | | |
| 60 | B | | | | | | | | |
| 61 | B | | | | | | | | |
| 62 | B | | | | | | | | |
| 64 | C | | | | | | | | |
| 76 | B | | | | | | | | |
| 77 | B | | | | | | | | |
| 78 | B | | | | | | | | |
| 103 | C | | | | | | | | |
| 104 | C | | | | | | | | |
| 125 | C | | | | | | | | |
| 139 | | C | C | C | C | C | C | C | |
| 148 | B | B | C | C | C | C | C | C | |
| 152 | C | | | | | | | | |
| 153 | C | C | C | C | C | C | C | C | |
| 154 | C | | | | | | | | |
| 155 | C | | | | | | | | |
| 156 | B | | | | | | | | |
| 157 | C | C | C | C | C | C | C | C | |
| 158 | | C | C | C | C | C | C | C | |
| 159 | | C | C | C | C | C | C | C | |
| 160 | C | | | | | | | | |
| 161 | C | | | | | | | | |
| 168 | | B | B | B | B | C | C | C | |
| 172 | C | | | | | | | | |
| 183 | | A | A | B | B | C | C | C | |
| 185 | | A | A | A | B | C | C | C | |
| 186 | | B | | | | | | | C |
| 187 | | | | | | | | | |
| 188 | | | | | | | | | |
| 189 | | | | | | | | | |
| 190 | | | | | | | | | |
| 191 | | | | | | | | | |
| 192 | B | | | | | | | | B |
| 193 | B | | | | | | | | B |
| 194 | | | | | | | | | |
| 195 | | | | | | | | | |
| 196 | C | | | | | | | | |
| 197 | | | | | | | | | |
| 198 | | | | | | | | | |
| 199 | | | | | | | | | |
| 200 | | | | | | | | | |
| 201 | | | | | | | | | |
| 202 | | | | | | | | | |
| 203 | | | | | | | | | |
| 204 | | | | | | | | | |
| 205 | C | | | | | | | | |
| 206 | B | | | | | | | | C |
| 207 | B | | | | | | | | |
| 208 | B | | | | | | | | |
| 209 | C | | | | | | | | |
| 210 | B | | | | | | | | |
| 211 | | | | | | | | | |
| 212 | | | | | | | | | |
| 213 | | | | | | | | | C |
| 214 | | | | | | | | | |
| 215 | C | | | | | | | | |
| 216 | | | | | | | | | |
| 217 | | | | | | | | | |
| 218 | | | | | | | | | |
| 219 | | | | | | | | | C |
| 220 | | | | | | | | | C |
| 221 | | | | | | | | | |
| 222 | | | | | | | | | |
| 223 | | | | | | | | | B |
| 224 | B | | | | | | | | C |
| 225 | B | | | | | | | | C |
| 226 | | | | | | | | | |
| 227 | C | | | | | | | | C |
| 228 | | | | | | | | | C |
| 229 | | | | | | | | | C |
| 230 | | | | | | | | | C |
| 231 | | | | | | | | | C |
| 232 | | | | | | | | | C |
| 233 | | | | | | | | | C |
| 234 | | | | | | | | | C |
| 235 | | | | | | | | | C |
| 236 | | | | | | | | | C |
| 237 | | | | | | | | | C |
| 238 | | | | | | | | | |
| 239 | | | | | | | | | |
| 240 | B | | | | | | | | B |
| 241 | | | | | | | | | C |
| 242 | | | | | | | | | |
| 243 | | | | | | | | | |
| 244 | B | | | | | | | | B |
| 245 | B | | | | | | | | C |
| 246 | B | | | | | | | | C |
| 247 | C | | | | | | | | C |
| 248 | | | | | | | | | B |
| 249 | | | | | | | | | |
| 250 | | | | | | | | | C |
| 251 | | | | | | | | | C |
| 252 | | | | | | | | | C |
| 253 | | | | | | | | | C |
| 254 | | | | | | | | | C |
| 255 | | | | | | | | | B |
| 256 | | | | | | | | | C |
| 257 | | | | | | | | | B |
| 258 | | | | | | | | | B |
| 259 | | | | | | | | | A |
| 260 | | | | | | | | | C |
| 261 | | | | | | | | | B |
| 262 | | | | | | | | | B |
| 263 | | | | | | | | | C |
| 264 | | | | | | | | | B |
| 265 | | | | | | | | | C |
| 266 | | | | | | | | | B |
| 267 | | | | | | | | | |
| 268 | | | | | | | | | C |
| 269 | | | | | | | | | C |
| 270 | | | | | | | | | B |
| 271 | | | | | | | | | B |
| 272 | | | | | | | | | A |
| 273 | | | | | | | | | B |
| 274 | | | | | | | | | C |
| 275 | | | | | | | | | B |
| 276 | | | | | | | | | C |
| 277 | | | | | | | | | C |
| 278 | | | | | | | | | B |
| 279 | | | | | | | | | B |
| 280 | | | | | | | | | B |
| 281 | | | | | | | | | B |
| 282 | | | | | | | | | B |
| 283 | | | | | | | | | A |
| 284 | | | | | | | | | B |
| 285 | | | | | | | | | B |
| 286 | | | | | | | | | C |
| 287 | | | | | | | | | C |
| 288 | | | | | | | | | C |
| 289 | | | | | | | | | C |
| 290 | | | | | | | | | C |
| 291 | | | | | | | | | |
| 292 | | | | | | | | | |
| 293 | | | | | | | | | |
| 294 | | | | | | | | | |
| 295 | | | | | | | | | |
| 296 | | | | | | | | | |
| 297 | | | | | | | | | |
| 298 | | | | | | | | | |
| 299 | | | | | | | | | |
| 300 | | | | | | | | | |
| 301 | | | | | | | | | |
| 302 | | | | | | | | | |
| 303 | | | | | | | | | |
| 304 | B | | | | | | | | |
| 305 | | | | | | | | | |
| 306 | C | | | | | | | | |
| 307 | B | | | | | | | | |
| 308 | B | | | | | | | | |
| 309 | | | | | | | | | |
| 310 | | | | | | | | | |
| 311 | | | | | | | | | |
| 312 | | | | | | | | | |
| 313 | B | | | | | | | | |
| 314 | B | | | | | | | | |
| 315 | | | | | | | | | |
| 316 | | | | | | | | | |
| 317 | | | | | | | | | |
| 318 | | | | | | | | | |
| 319 | | | | | | | | | B |
| 320 | C | | | | | | | | |
| 321 | | | | | | | | | |
| 322 | | | | | | | | | |
| 323 | B | | | | | | | | |
| 324 | B | | | | | | | | |
| 325 | | | | | | | | | |
| 326 | C | | | | | | | | B |
| 327 | | | | | | | | | |
| 328 | | | | | | | | | |
| 329 | | | | | | | | | |
| 330 | | | | | | | | | |
| 331 | | | | | | | | | |
| 332 | | | | | | | | | |
| 333 | | | | | | | | | |
| 334 | B | | | | | | | | B |
| 335 | | | | | | | | | |
| 336 | | | | | | | | | C |
| 337 | | | | | | | | | |
| 338 | | | | | | | | | |
| 339 | | | | | | | | | |
| 340 | | | | | | | | | |
| 341 | | | | | | | | | |
| 342 | | | | | | | | | |
| 343 | | | | | | | | | |
| 344 | | | | | | | | | |
| 345 | | | | | | | | | |
| 346 | | | | | | | | | B |
| 347 | | | | | | | | | C |
| 348 | | | | | | | | | B |
| 349 | | | | | | | | | B |
| 350 | | | | | | | | | B |
| 351 | | | | | | | | | B |

¹ERBB2 flow cytometry protein degradation assay in BT-474 cells: A. DC50 < 100 nM; B. DC50 = 100-1000 nM; C. DC50 > 1000 nM;
²KRAS(G12C) Western blot protein degradation assay in MIA PaCa-2 cells: A. >66% degradation; B. 33-66% degradation; C. <33% degradation;
³KRAS(G12C) Western blot protein degradation assay in LU65A cells: A. DC50 < 100 nM; B. DC50 = 100-1000 nM; C. DC50 > 1000 nM

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A compound of the Formula I 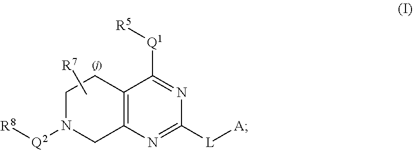
or a pharmaceutically acceptable salt thereof, wherein,
A is a chemical moiety that binds HSP90 protein;
L is a linker;
Q¹ is a nitrogen containing heteroaryl or heterocyclyl ring, each of which are optionally substituted with 1 to 3 groups selected from R⁶;
R⁵ is —C(O)Y or —S(O)₂Y;
Y is a (C₁-C₆)alkyl, halo(C₁-C₆)alkyl, (C₂-C₆)alkenyl, halo(C₂-C₆)alkenyl, NH₂, —NH(C₁-C₆)alkyl, —N[(C₁-C₆)alkyl]2, NHNH₂, or NHOH, wherein said (C₂-C₆)alkenyl, alone or as recited in halo(C₂-C₆)alkenyl, is optionally substituted with (C₁-C₆)alkyl, halo(C₁-C₆)alkyl, heteroalkyl, hydroxy(C₁-C₆)alkyl, —C(O)NH₂, —C(O)NH(C₁-C₆)alkyl, or —C(O)N[(C₁-C₆)alkyl]2;
R⁶ is (C₁-C₆)alkyl, halo(C₁-C₆)alkyl, (C₁-C₆)alkoxy, halo(C₁-C₆)alkoxy, hydroxy(C₁-C₆)alkyl, cyano(C₁-C₆)alkyl, oxo, cyano, heteroalkyl, —C(O)OH, —C(O)O(C₁-C₆)alkyl, —C(O)NH₂, —C(O)NH(C₁-C₆)alkyl, or —C(O)N[(C₁-C₆)alkyl]2, wherein said (C₁-C₆)alkyl is optionally substituted with heteroaryl;
R⁷ is halo, hydroxyl, (C₁-C₆)alkyl, halo(C₁-C₆)alkyl, (C₁-C₆)alkoxy, halo(C₁-C₆)alkoxy, cycloalkyl, heteroalkyl, hydroxy(C₁-C₆)alkyl, or S(C₁-C₆)alkyl;
j is 1 or 2;
Q² is a bond, —C(O)—, or (C₁-C₃)alkylene;
R⁸ is cycloalkyl, heterocyclyl, aryl, or heteroaryl, each of which are optionally substituted with 1 to 3 groups selected from R⁹;
R⁹ is halo, (C₁-C₆)alkyl, (C₂-C₆)alkenyl, halo(C₂-C₆)alkenyl, (C₂-C₆)alkynyl, oxo, cyano, —(C₁-C₆)alkylOR^{c}, —(C₁-C₆)alkylN(R^{d})₂, —(C₁-C₆)alkylC(O)OR^{d}, OH, —(C₁-C₆)alkylC(O)N(R^{d})₂, —(C₁-C₆)alkylO(C₁-C₆)alkylN(R^{d})₂, —(C₁-C₆)alkylSOR^{d}, —(C₁-C₆)alkylS(O)₂R^{d}, —(C₁-C₆)alkylSON(R^{d})₂, —(C₁-C₆)alkylSO₂N(R^{d})₂, —(C₁-C₆)alkylcycloalkyl, —(C₁-C₆)alkylheterocyclyl, —(C₁-C₆)alkylheteroaryl, —(C₁-C₆)alkylaryl, —(C₁-C₆)alkoxy, halo(C₁-C₆)alkoxy, CN, aryl, heteroaryl, cycloalkyl, heterocycloalkyl, —C(O)R^{d}, —C(O)OR^{d}, —C(O)N(R^{d})₂, N(R^{d})₂, —C(O)NR^{d}(C₁-C₆)alkylN(R^{d})₂, —NR^{d}(C₁-C₆)alkylN(R^{d})₂, —NR^{d}(C₁-C₆)alkylOR^{d}, —SOR^{d}, —S(O)₂R^{d}, —SON(R^{d})₂, —SO₂N(R^{d})₂, or CN, wherein each aryl, cycloalkyl, heterocyclyl, and heteoaryl alone and in connection with —(C₁-C₆)alkylcycloalkyl, —(C₁-C₆)alkylheterocyclyl, —(C₁-C₆)alkylheteroaryl, —(C₁-C₆)alkylaryl are optionally substituted with 1 to 3 groups selected from R^{e};
R^{c} and R^{d} are each independently selected from hydrogen, (C₁-C₆)alkyl, and halo(C₁-C₆)alkyl; and
R^{e} is selected from halo, oxo, CN, NO₂, —N(R^{d})₂, —OR^{d}, —C(O)OR^{d}, (C₁-C₆)alkyl, —(C₁-C₆)alkylOR^{c}, halo(C₁-C₆)alkyl, (C₁-C₆)alkoxy, halo(C₁-C₆)alkoxy, —(C₁-C₆)alkylC(O)OR^{d}, —(C₁-C₆)alkylC(O)N(R^{d})₂, (C₂-C₆)alkenyl, halo(C₂-C₆)alkenyl, (C₂-C₆)alkynyl, —(C₁-C₆)alkylSR^{d}, —(C₁-C₆)alkylOR^{c}, —(C₁-C₆)alkylN(R^{d})₂, —C(O)N(R^{d})₂, —C(O)NR^{d}C₁₋₆alkylN(R^{d})₂, —NR^{d}C₁-₆alkylN(R^{d})₂, —NR^{d}C₁₋₆alkylOR^{d}, —SOR^{d}, —S(O)₂R^{d}, —SON(R^{d})₂, —SO₂N(R^{d})₂, aryl, heteroaryl, cycloalkyl, and heterocycloalkyl.

2. compound of claim 1, or a pharmaceutically acceptable salt thereof, wherein A is selected from 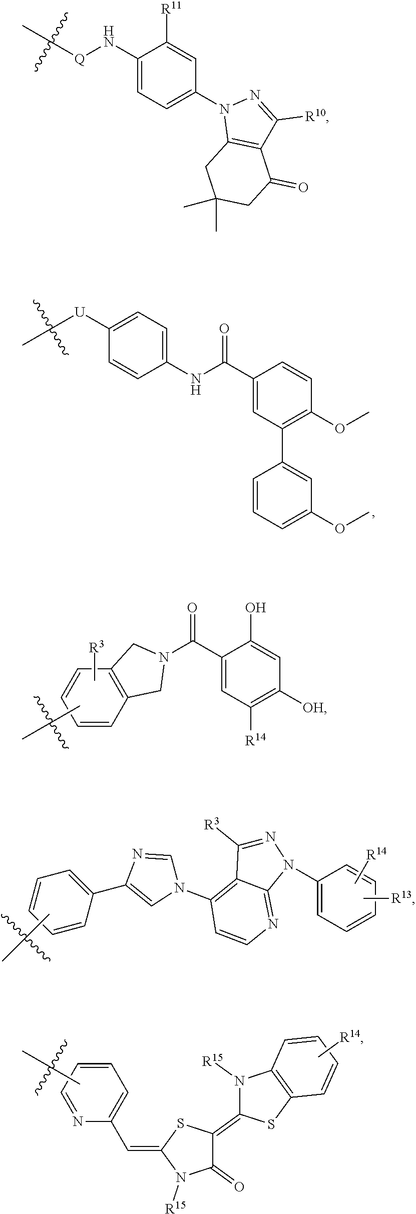 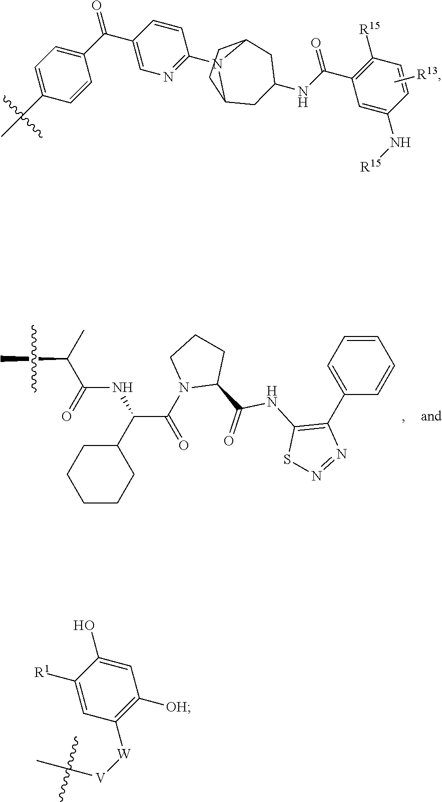
wherein
Q and U are each independently selected from phenyl, heteroaryl, heterocyclyl, and cycloalkyl, each of which being optionally substituted with 1 to 3 groups selected from R²;
R¹³ and R¹⁴ are each independently selected from hydrogen, halo, —CN, (C₁-C₄)alkyl, halo(C₁-C₄)alkyl, and —C(O)NR^{a}R;
R¹⁵ is hydrogen, (C₁-C₄)alkyl, or halo(C₁-C₄)alkyl;
W is 5- or 6-membered heteroaryl optionally substituted with 1 to 3 groups selected from R²;
V is phenyl or 5- to 9-membered heteroaryl optionally substituted with 1 to 3 groups selected from R³;
R¹ is halo, (C₁-C₄)alkyl, halo(C₁-C₄)alkyl, (C₁-C₄)alkoxy, or halo(C₁-C₄)alkoxy;
R² is (C₁-C₄)alkyl, halo(C₁-C₄)alkyl, (C₂-C₆)alkenyl, halo(C₂-C₆)alkenyl, (C₂-C₆)alkynyl, halo(C₂-C₆)alkynyl, CN, —C₁₋₄alkylOR^{a}, —OR^{a}, —C(O)R^{a}, —C(O)OR^{a}, —C(O)NR^{a}R^{b}, —C(O)NR^{a}(C₁₋₄alkylene)OR^{a}, —C(O)NR^{a}(C₁₋₄alkylene)NR^{a}R^{b}, —C(O)NR^{a}(C₁₋₄alkylene)OR, —NR^{a}R^{b}, —O(C₁₋₄alkylene)NR^{a}R^{b}, —SH, —S(C₁₋₄alkyl), —C₁₋₄alkylNR^{a}R^{b}, —SR^{a}, —S(O)R^{a}, —S(O)₂R^{a}, —S(O)NR^{a}R^{b}, —SO₂NR^{a}R^{b}, —NR^{a}(C₁₋₄alkyl)OR^{a}, —NR^{a}(C₁₋₄alkyl)NR^{a}R^{b}, —C₁₋₆alkylC(O)NR^{a}R^{b}, phenyl or 5- to 7-membered heteroaryl, wherein said phenyl and 5- to 7-membered heteroaryl are each optionally and independently substituted with 1 to 3 groups selected from R⁴;
R^{a} and R^{b} are each independently selected from hydrogen and (C₁-C₄)alkyl, wherein said (C₁-C₄)alkyl is optionally substituted with one or more halo or a 3- to 7-membered heterocyclyl, or both; and
R³ and R⁴ are each independently halo, —NR^{a}R^{b}, (C₁-C₄)alkyl, halo(C₁-C₄)alkyl, (C₁-C₄)alkoxy, or halo(C₁-C₄)alkoxy.

3. The compound of claim 2, or a pharmaceutically acceptable salt thereof, wherein A is

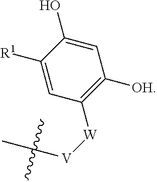

4. The compound of claim 3, wherein the compound is of the Formula 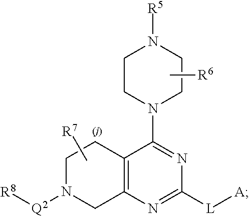
or a pharmaceutically acceptable salt thereof.

5. The compound of claim 4, or a pharmaceutically acceptable salt thereof, wherein is R⁵ is —C(O)Y: Y is (C₁-C₆)alkyl, halo(C₁-C₆)alkyl, (C₂-C₆)alkenyl, halo(C₂-C₆)alkenyl, or NH₂; and R⁶ is cyano(C₁-C₆)alkyl.

6-10. (canceled)

11. The compound of claim 5, or a pharmaceutically acceptable salt thereof, wherein j is 0 and Q² is a bond.

12. (canceled)

13. The compound of claim 5, or a pharmaceutically acceptable salt thereof, wherein R⁸ is aryl optionally substituted with 1 to 3 groups selected from R⁹; and R⁹ is selected from halo, (C₁-C₆)alkyl, and OH.

14-16. (canceled)

17. The compound of claim 4, or a pharmaceutically acceptable salt thereof, wherein A is selected from 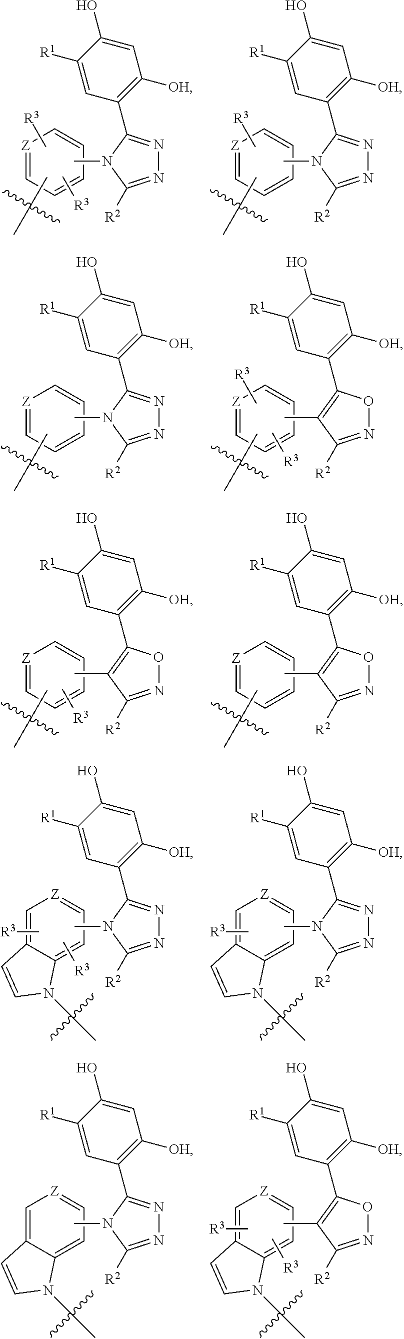 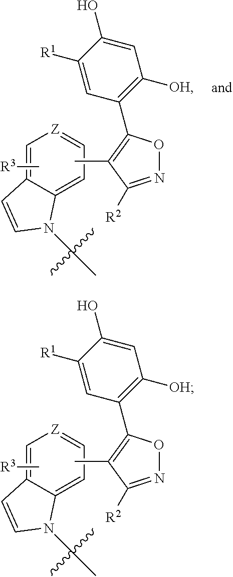
and
Z is N or CH.

18-20. (canceled)

21. The compound of claim 4, or a pharmaceutically acceptable salt thereof, wherein A is

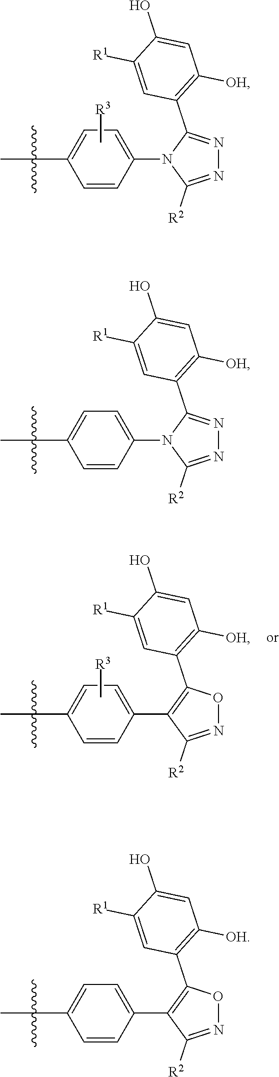

22. (canceled)

23. (canceled)

24. The compound of claim 21, or a pharmaceutically acceptable salt thereof, wherein R¹ is halo or (C₁-C₄)alkyl; R² is —OR^{a}, —SR^{a}, —C(O)NR^{a}R^{b}, or —C(O)NR^{a}(C₁₋₄alkylene)NR^{a}R; and R^{a} and R^{b} are each independently selected from hydrogen and (C₁-C₄)alkyl, wherein said (C₁-C₄)alkyl is optionally substituted with 1 to 3 halo or a 6-membered heterocyclyl.

25-31. (canceled)

32. The compound of claim 1, or a pharmaceutically acceptable salt thereof, wherein
L is -Het¹-X¹—*, -Het¹-Het²-X¹—*, *—X¹-Het¹-X²-Het²-(CH₂)ₘO—, —NR^{c}—(CH₂)ₘ—X¹-Het¹-X²—*, —NR^{c}—(CH₂)ₘ—X—NR^{c}—(CH₂CH₂O)₁—*, —NR^{c}—(CH₂)ₘ—X—NR^{c}—(CH₂)ₚ—*, —NR^{c}—(CH₂)ₘ—X¹-Het¹-X²-Het²-X³—*, —O(CH₂)ₘ—X¹-Het¹-X²-Het²-X³—*, —O(CH₂)ₘ—X¹—NR^{c}—(CH₂)ₚ-Het¹- X²-Het²-X³—*, *—X¹—NR^{c}—(CH₂)ₘ-Het¹-X²-Het²-X³—(CH₂)ₚ—NR^{d}—(CH₂)ₚ—, —NR^{c}—(CH₂)ₘ—X—(CH)CH₃-Het¹-X²-Het³-X³—*, —NR^{c}—(CH₂)ₘ—X¹—(CH₂)ₚ-Het¹-X²-Het²-X³—*, —NR^{c}—(CH₂)ₘ—X¹—NR^{d}—(CH₂)ₚ-Het¹-X²-Het²-X³—*, —NR^{c}—(CH₂)ₘ—NR^{d}—X-Het¹-X²—*, *Het-X¹-Het²-X²—, *-Het-X¹-Het²-X²—O—, *—O(CH₂)ₘ-Het⁴-(CH₂)ₚ—O(CH₂)ₘ—NR^{c}—X²—, —O(CH₂)ₘ-Het¹-(CH₂)ₚ—O(CH₂)ₘ—NR^{c}—X²—*, *-Het¹-O—(CH₂)ₘ—X¹-Het²-X²—, *-Het¹-O—(CH₂)ₘ—X—NR^{c}—(CH₂CH₂O)ₙ(CH₂)ₘ-Het²-X²—, *-Het¹-X¹—NR^{c}—(CH₂)ₘ—, *-Het¹-X¹-Het²-Het³-X²—, *-Het¹-X—NR—(CH₂CH₂O)ₙ(CH₂)ₘ—, *-Het¹-X¹—NR^{c}—(CH₂CH₂O)ₙHet²-(CH₂)ₘ—X²—, *-Het¹-X—NR^{c}—(CH₂CH₂O)ₙ—, *-Het¹-X¹—NR^{c}—(CH₂)ₘ-Het²-X²—Het³-(CH₂)ₘ—, *-Het¹-X-Het²-(CH₂)ₘ-Het-X²—, *-Het-X¹-Het²-, *-Het¹-X¹—NR^{c}—, *-Het-XL-NR^{c}—(CH₂)ₘ-Phe-X²-Het²-(CH₂)ₘ—, *-Het¹-X¹-Het²-Het³-, *-Het¹-X¹-Het²-(CH₂)ₘ-Het³-X²—(CH₂)ₚ—NR^{c}—(CH₂)ₘ—, *-Het¹-X¹-Het²-(CH₂)ₘ-Het³-(CH₂)ₘ—O—, *-Het¹-X¹-Het²-(CH₂)ₘ- Het³-(CH₂)ₚ—NR^{c}—(CH₂)ₘ—, *-Het¹-X¹-Het²-(CH₂CH₂O)ₙ—,*-Het¹-X¹—(CH₂)ₘ-Het²-X²—, *—(CH₂CH₂O)ₒ—(CH₂)ₚ-Het¹-X¹-Het²-(CH₂CH₂O)ₙ, *—(CH₂CH₂O)ₙ—(CH₂)ₘ-Het¹-X¹-Het²-X², *- Het¹-X¹-Phe-X²—NR^{c}—X³—, *—(CH₂CH₂O)ₒ—(CH₂)ₚ-Het¹-X¹-Phe-X²—NR^{c}—(CH₂CH₂O)ₙ—, *—(CH₂CH₂O)ₙ—(CH₂)ₘ—NR^{c}-Phe-X¹—, *—(CH₂CH₂O)ₒ—(CH₂)ₚ—NR^{c}-Phe-(CH₂CH₂O)ₙ—, *—(CH₂CH₂O)ₒ—(CH₂)ₚ—NR^{c}—(CH₂CH₂O)ₙ—(CH₂)ₘ—, *—(CH₂CH₂O)ₙ—(CH₂)ₘ—NR^{c}—(CH₂CH₂O)ₙ—(CH₂)ₘ—C(O)—NR^{d}—(CH₂CH₂O)ₒ—(CH₂)ₚ—, *—(CH₂CH₂O)ₒ—(CH₂)ₚ—NR^{c}—(CH₂CH₂O)ₙ—(CH₂)ₘ-Het¹-X¹-Het²-X²—, *—(CH₂CH₂O)ₒ—(CH₂)ₚ—NR^{c}—(CH₂CH₂O)—(CH₂)ₘ-Het¹-X¹-Het²-X²—; (CH₂CH₂O)ₒ, *—NR^{c}—(CH₂CH₂O)ₙ—(CH₂)ₘ-Phe-NH-XL-Het¹-X², *—NR^{c}—(CH₂CH₂O)ₙ—(CH₂)ₘ-Phe-NH—X¹-Het¹-X²—(CH₂CH₂O)ₒ, *—(CH₂CH₂O)ₒ—(CH₂)ₚ—NR^{c}—(CH₂CH₂O)—(CH₂)ₘ-Phe-X¹—NR^{c}—(CH₂CH₂O)ₒ—(CH₂)ₚ—, *—(CH₂CH₂O)ₒ—(CH₂)ₚ—NR^{c}—(CH₂CH₂O)ₙ—(CH₂)ₘ-Het¹-X¹—, *—(CH₂CH₂O)ₒ—(CH₂)ₚ—NR^{c}—(CH₂CH₂O)ₙ—(CH₂)ₘ-Het¹-X¹—(CH₂CH₂O)ₙ—, *—(CH₂CH₂O)ₙ—(CH₂)ₘ—NR^{c}—(CH₂)ₘ—C(O)—NR^{d}-Het¹-X-Het²-(CH₂CH₂O)ₒ-(CH₂)ₚ, or *—NR^{c}—(CH₂)ₘ—C(O)—NR^{d}—(CH₂)ₘ-Het¹-X¹-Het²-X²—;
* indicates the point of attachment to A;
Het¹, Het², and Het³ are each independently phenyl, a 4- to 6-membered heterocyclyl, 5-to 7-membered heteroaryl, or a 4- to 6-membered cycloalkyl, each of which are optionally substituted with (C₁-C₄)alkyl;
X¹, X², and X³, are each independently C(O) or (CH₂)ᵣ; and
m, n, o, p, q and r are each independently integers selected from 0, 1, 2, 3, 4, 5, and 6.

33. (canceled)

34. The compound of claim 32, or a pharmaceutically acceptable salt thereof, wherein L is —NR^{c}—(CH₂)ₘ—X¹—NR^{c}—(CH₂)ₚ—* or —NR—(CH₂)ₘ—X¹-Het¹-X²-Het²-X³—*.

35. The compound of claim 32, or a pharmaceutically acceptable salt thereof, wherein Het¹ and Het² are each independently phenyl or a 4- to 6-membered heterocyclyl.

36. (canceled)

37. (canceled)

38. The compound of claim 1, or a pharmaceutically acceptable salt thereof, wherein L is selected from

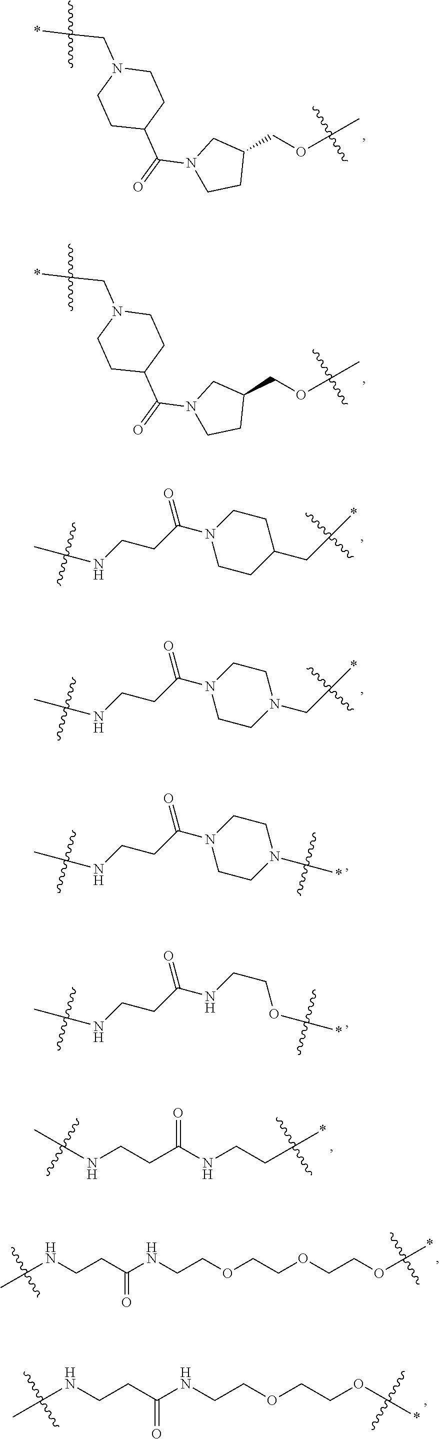

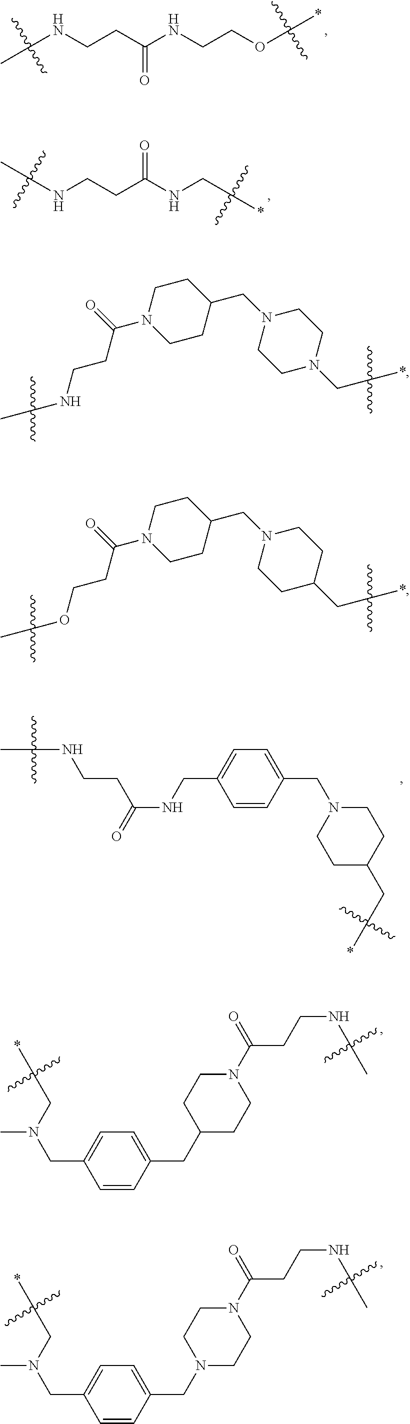

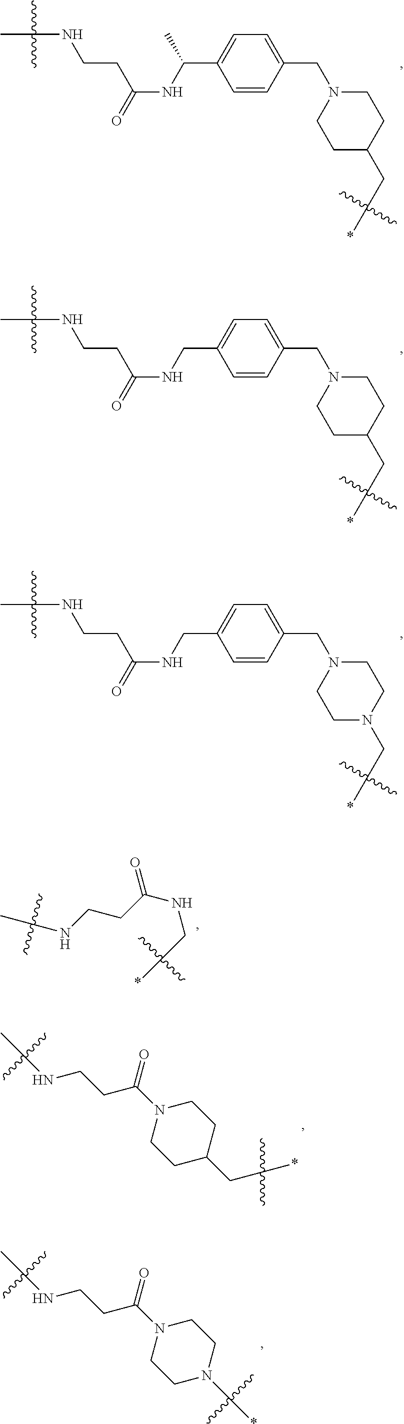

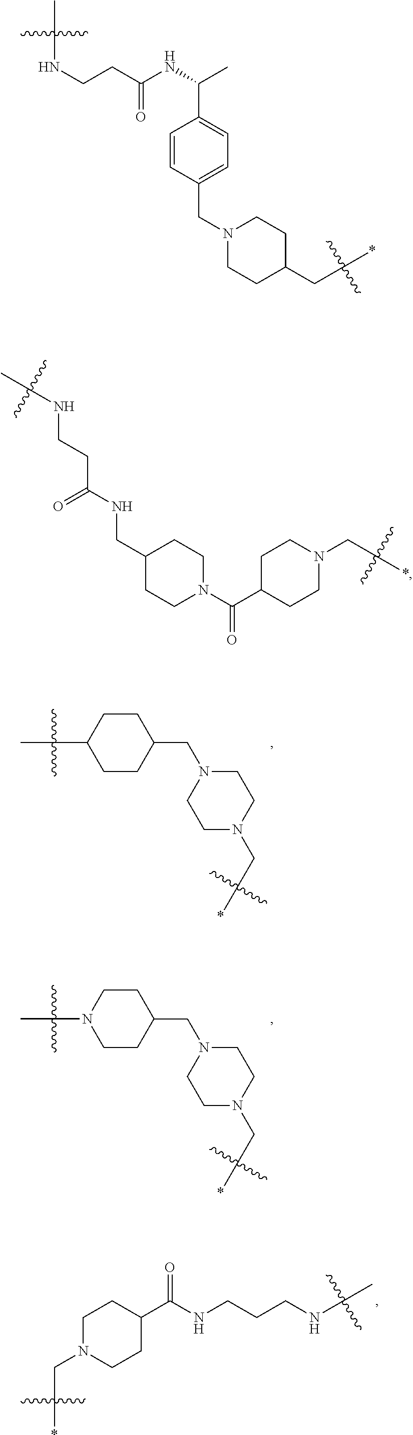

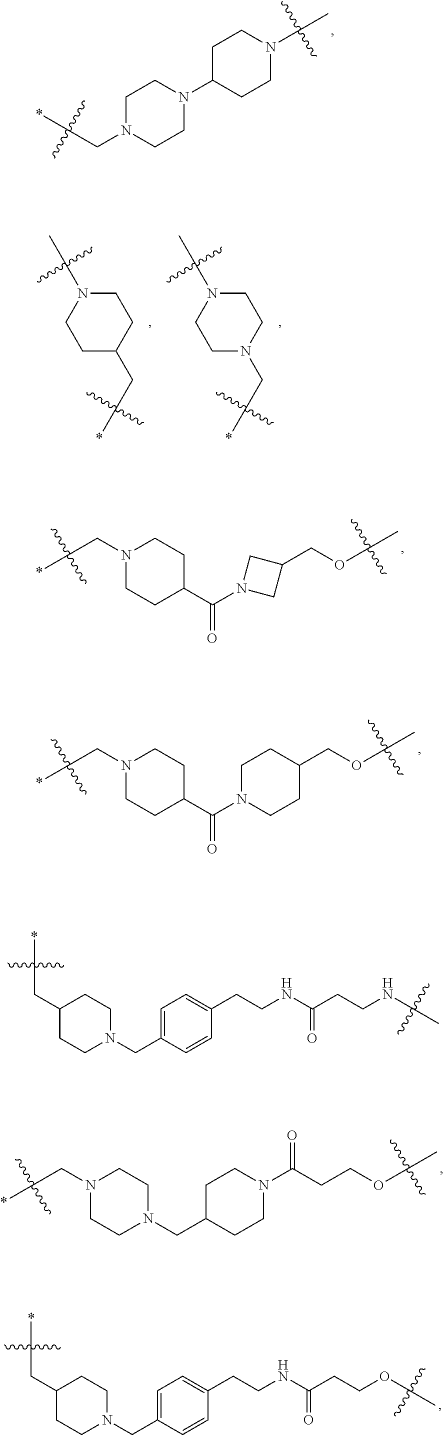

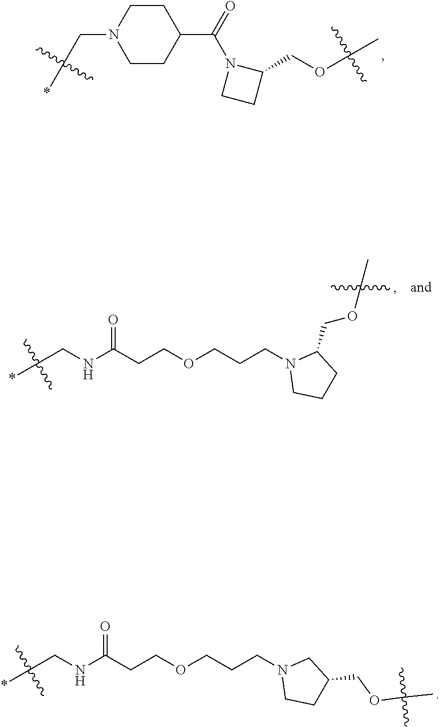

39. The compound of claim 38, or a pharmaceutically acceptable salt thereof, wherein L is selected from

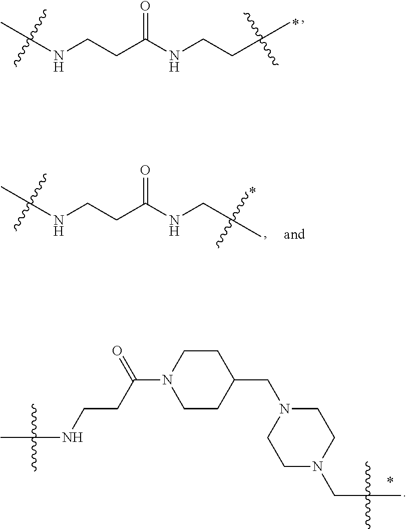 [SMILES: *CCNC(=O)CCN* ; *CNC(=O)CCN* ; *CN1CCN(CC2CCN(C(=O)CCN*)CC2)CC1]

40. The compound of claim 1, wherein the compound is selected from the following structural formula: 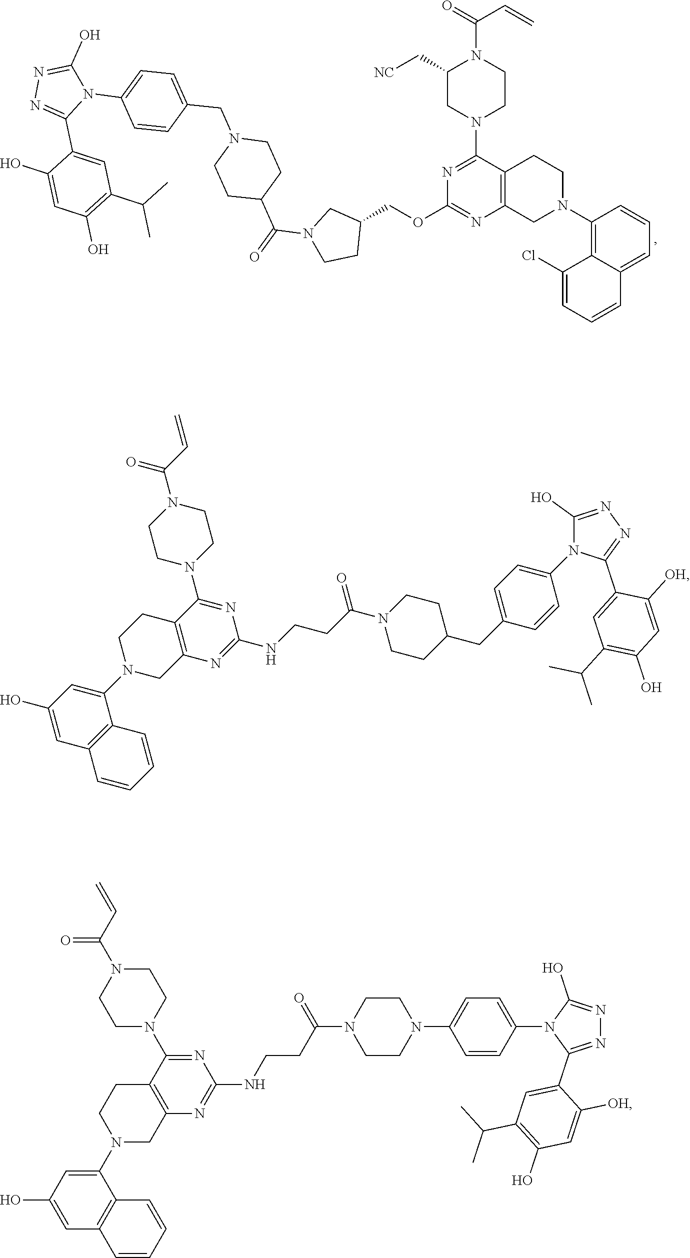 [SMILES: C=CC(=O)N1CCN(c2nc(OC[C@@H]3CCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N ; C=CC(=O)N1CCN(c2nc(NCCC(=O)N3CCC(Cc4ccc(-n5c(O)nnc5-c5cc(C(C)C)c(O)cc5O)cc4)CC3)nc3c2CCN(c2cc(O)cc4ccccc24)C3)CC1 ; C=CC(=O)N1CCN(c2nc(NCCC(=O)N3CCN(c4ccc(-n5c(O)nnc5-c5cc(C(C)C)c(O)cc5O)cc4)CC3)nc3c2CCN(c2cc(O)cc4ccccc24)C3)CC1] 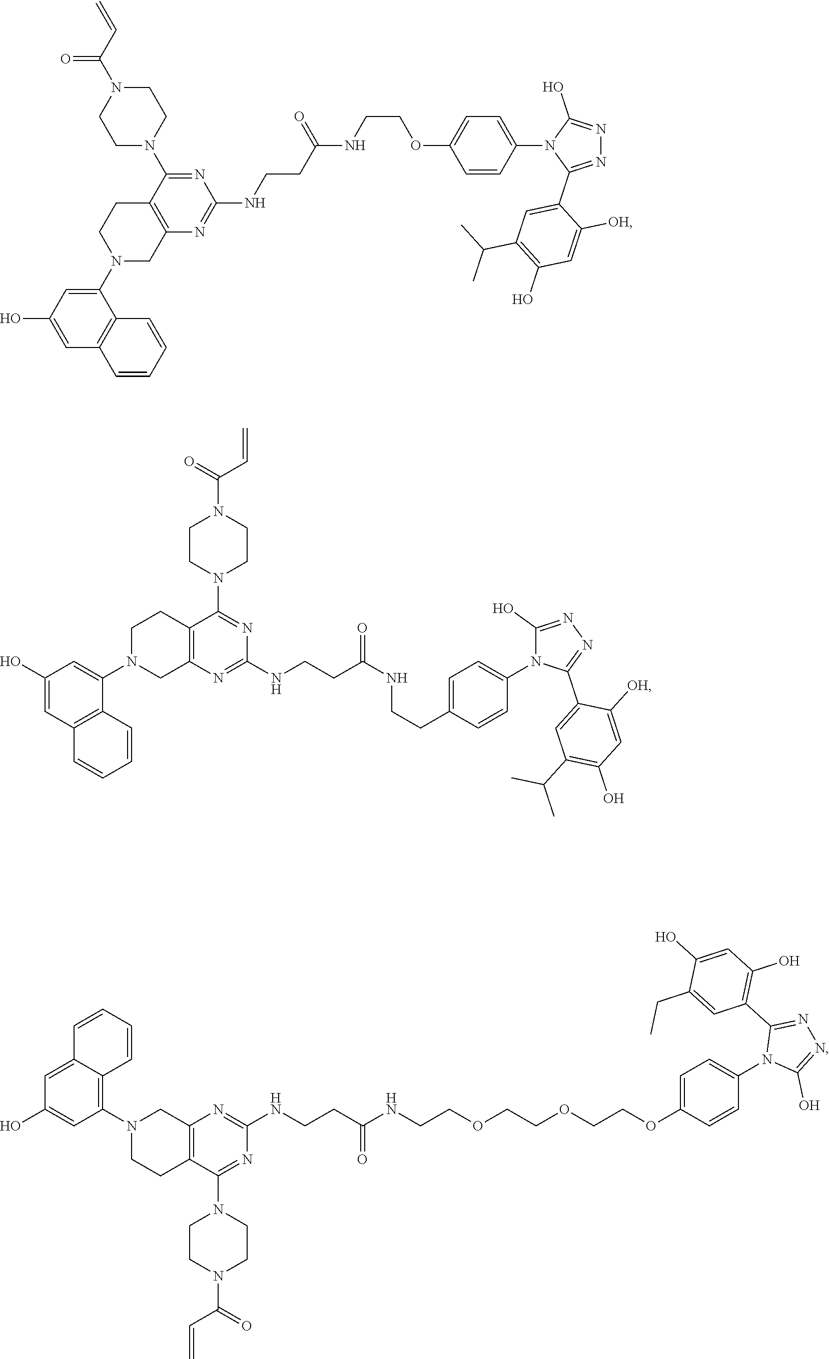 [SMILES: C=CC(=O)N1CCN(c2nc(NCCC(=O)NCCOc3ccc(-n4c(O)nnc4-c4cc(C(C)C)c(O)cc4O)cc3)nc3c2CCN(c2cc(O)cc4ccccc24)C3)CC1 ; C=CC(=O)N1CCN(c2nc(NCCC(=O)NCCc3ccc(-n4c(O)nnc4-c4cc(C(C)C)c(O)cc4O)cc3)nc3c2CCN(c2cc(O)cc4ccccc24)C3)CC1 ; C=CC(=O)N1CCN(c2nc(NCCC(=O)NCCOCCOCCOc3ccc(-n4c(O)nnc4-c4cc(CC)c(O)cc4O)cc3)nc3c2CCN(c2cc(O)cc4ccccc24)C3)CC1] 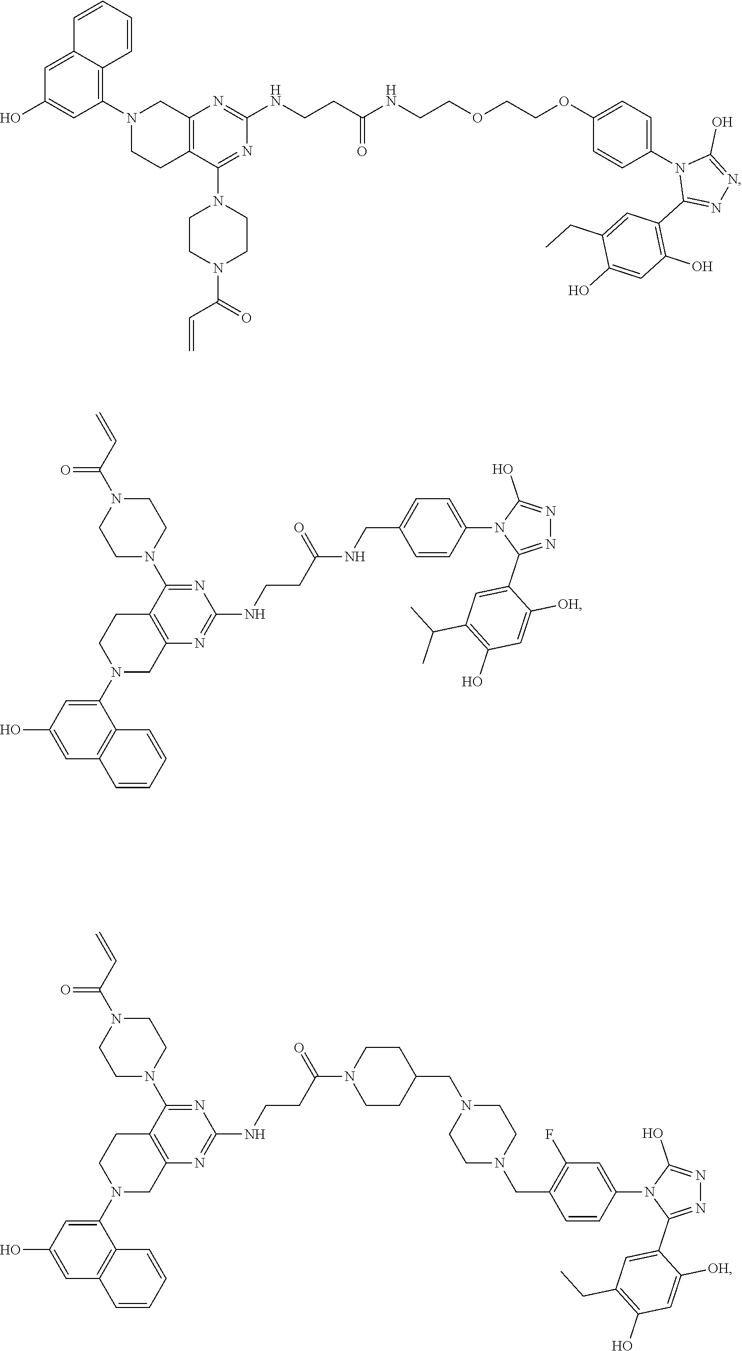 [SMILES: C=CC(=O)N1CCN(c2nc(NCCC(=O)NCCOCCOc3ccc(-n4c(O)nnc4-c4cc(CC)c(O)cc4O)cc3)nc3c2CCN(c2cc(O)cc4ccccc24)C3)CC1 ; C=CC(=O)N1CCN(c2nc(NCCC(=O)NCc3ccc(-n4c(O)nnc4-c4cc(C(C)C)c(O)cc4O)cc3)nc3c2CCN(c2cc(O)cc4ccccc24)C3)CC1 ; C=CC(=O)N1CCN(c2nc(NCCC(=O)N3CCC(CN4CCN(Cc5ccc(-n6c(O)nnc6-c6cc(CC)c(O)cc6O)cc5F)CC4)CC3)nc3c2CCN(c2cc(O)cc4ccccc24)C3)CC1] 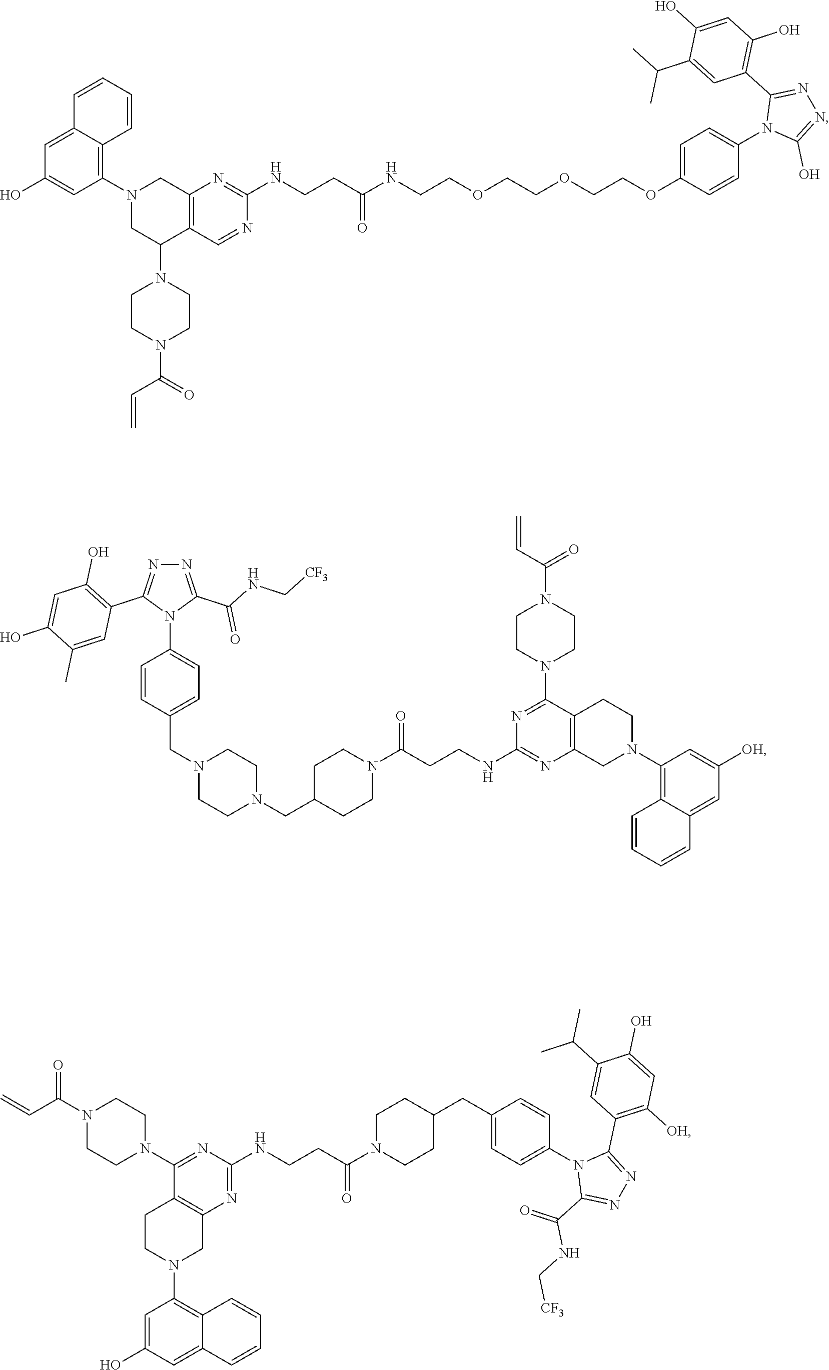 [SMILES: C=CC(=O)N1CCN(C2CN(c3cc(O)cc4ccccc34)Cc3nc(NCCC(=O)NCCOCCOCCOc4ccc(-n5c(O)nnc5-c5cc(C(C)C)c(O)cc5O)cc4)ncc32)CC1 ; C=CC(=O)N1CCN(c2nc(NCCC(=O)N3CCC(CN4CCN(Cc5ccc(-n6c(C(=O)NCC(F)(F)F)nnc6-c6cc(C)c(O)cc6O)cc5)CC4)CC3)nc3c2CCN(c2cc(O)cc4ccccc24)C3)CC1 ; C=CC(=O)N1CCN(c2nc(NCCC(=O)N3CCC(Cc4ccc(-n5c(C(=O)NCC(F)(F)F)nnc5-c5cc(C(C)C)c(O)cc5O)cc4)CC3)nc3c2CCN(c2cc(O)cc4ccccc24)C3)CC1] 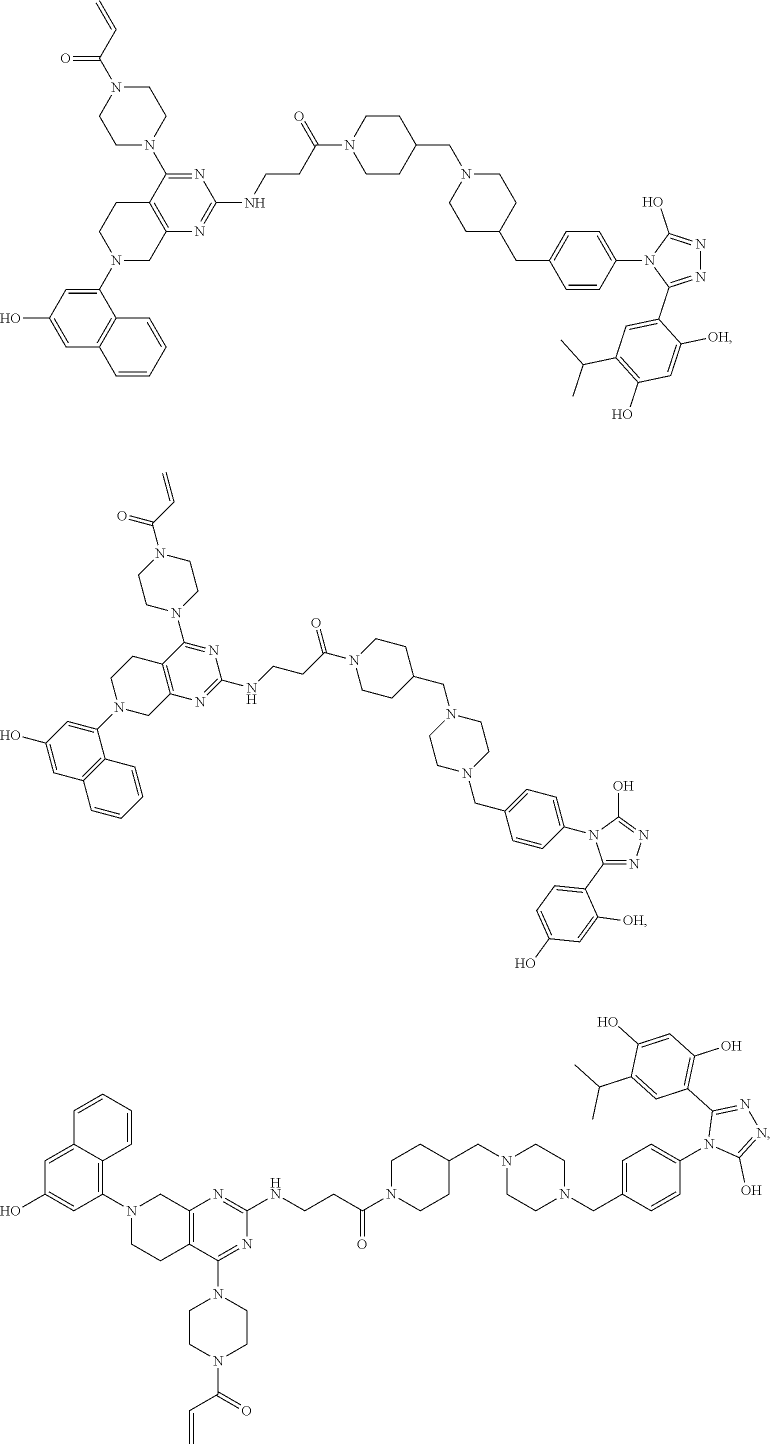 [SMILES: C=CC(=O)N1CCN(c2nc(NCCC(=O)N3CCC(CN4CCC(Cc5ccc(-n6c(O)nnc6-c6cc(C(C)C)c(O)cc6O)cc5)CC4)CC3)nc3c2CCN(c2cc(O)cc4ccccc24)C3)CC1 ; C=CC(=O)N1CCN(c2nc(NCCC(=O)N3CCC(CN4CCN(Cc5ccc(-n6c(O)nnc6-c6ccc(O)cc6O)cc5)CC4)CC3)nc3c2CCN(c2cc(O)cc4ccccc24)C3)CC1 ; C=CC(=O)N1CCN(c2nc(NCCC(=O)N3CCC(CN4CCN(Cc5ccc(-n6c(O)nnc6-c6cc(C(C)C)c(O)cc6O)cc5)CC4)CC3)nc3c2CCN(c2cc(O)cc4ccccc24)C3)CC1] 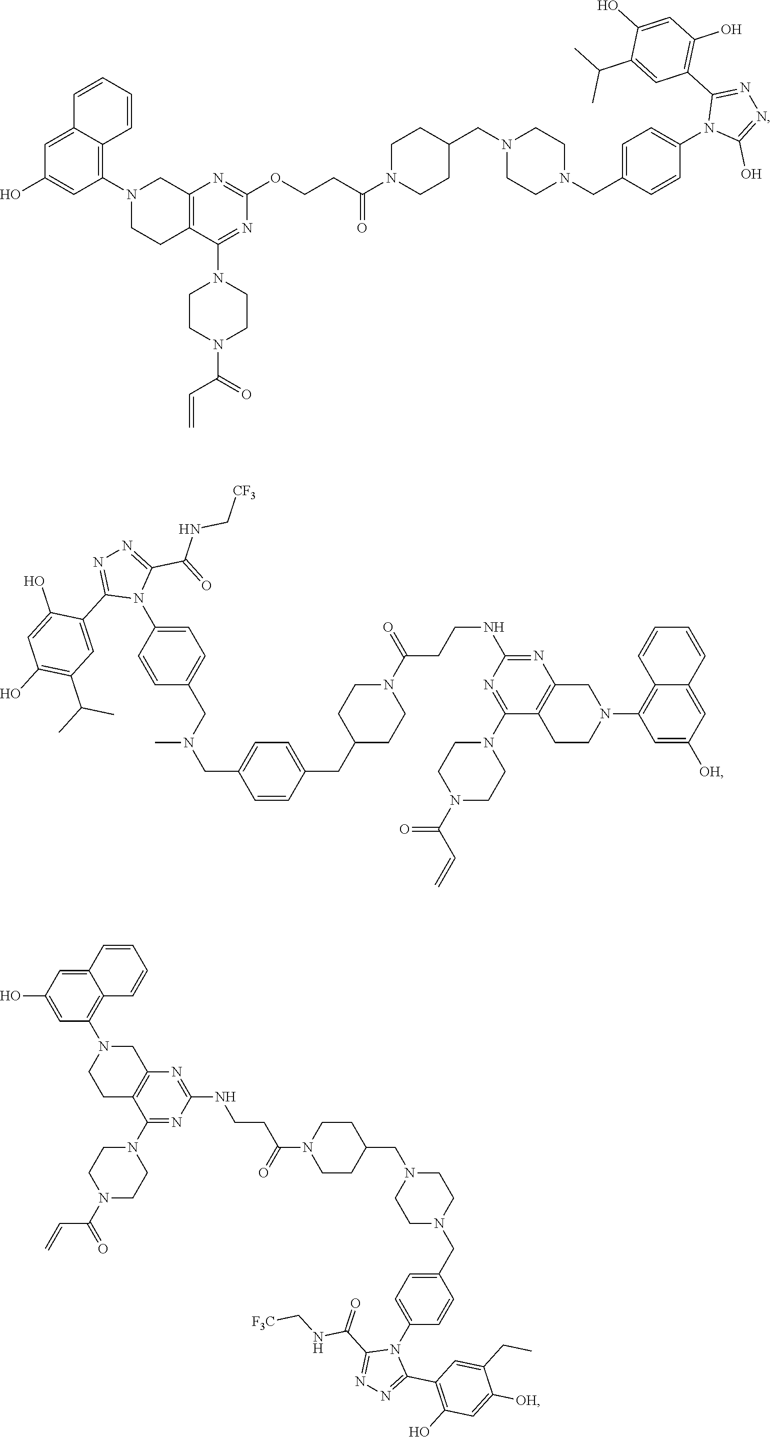 [SMILES: C=CC(=O)N1CCN(c2nc(OCCC(=O)N3CCC(CN4CCN(Cc5ccc(-n6c(O)nnc6-c6cc(C(C)C)c(O)cc6O)cc5)CC4)CC3)nc3c2CCN(c2cc(O)cc4ccccc24)C3)CC1 ; C=CC(=O)N1CCN(c2nc(NCCC(=O)N3CCC(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cc(O)cc4ccccc24)C3)CC1 ; C=CC(=O)N1CCN(c2nc(NCCC(=O)N3CCC(CN4CCN(Cc5ccc(-n6c(C(=O)NCC(F)(F)F)nnc6-c6cc(CC)c(O)cc6O)cc5)CC4)CC3)nc3c2CCN(c2cc(O)cc4ccccc24)C3)CC1] 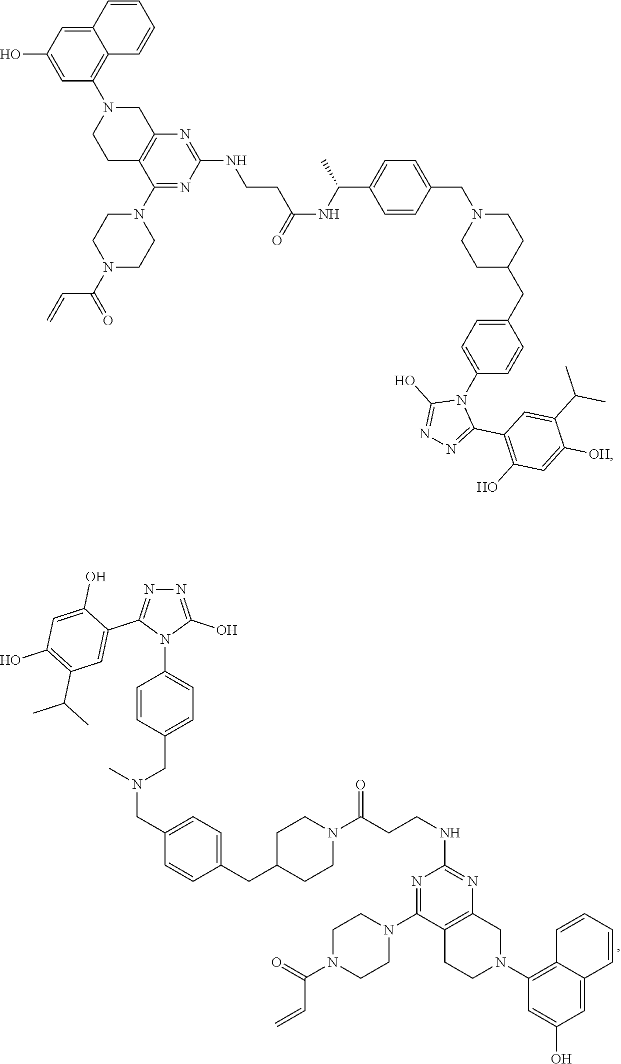 [SMILES: C=CC(=O)N1CCN(c2nc(NCCC(=O)N[C@H](C)c3ccc(CN4CCC(Cc5ccc(-n6c(O)nnc6-c6cc(C(C)C)c(O)cc6O)cc5)CC4)cc3)nc3c2CCN(c2cc(O)cc4ccccc24)C3)CC1 ; C=CC(=O)N1CCN(c2nc(NCCC(=O)N3CCC(Cc4ccc(CN(C)Cc5ccc(-n6c(O)nnc6-c6cc(C(C)C)c(O)cc6O)cc5)cc4)CC3)nc3c2CCN(c2cc(O)cc4ccccc24)C3)CC1] 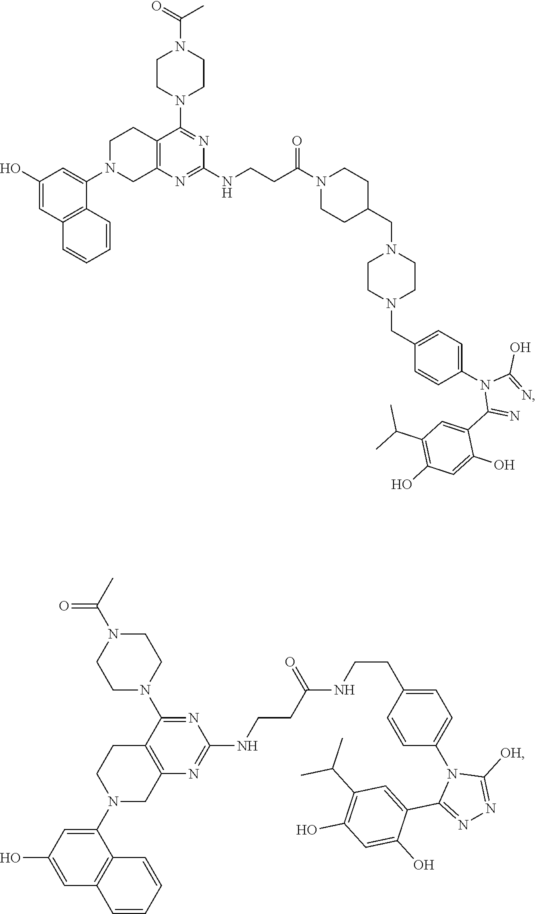 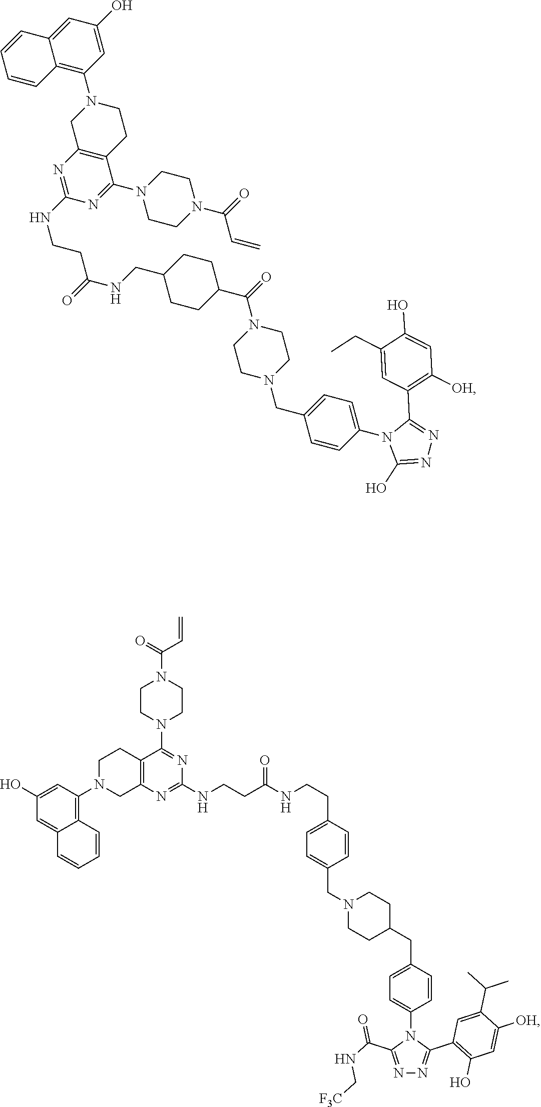 [SMILES: C=CC(=O)N1CCN(c2nc(NCCC(=O)NCC3CCC(C(=O)N4CCN(Cc5ccc(-n6c(O)nnc6-c6cc(CC)c(O)cc6O)cc5)CC4)CC3)nc3c2CCN(c2cc(O)cc4ccccc24)C3)CC1 ; C=CC(=O)N1CCN(c2nc(NCCC(=O)NCCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1] 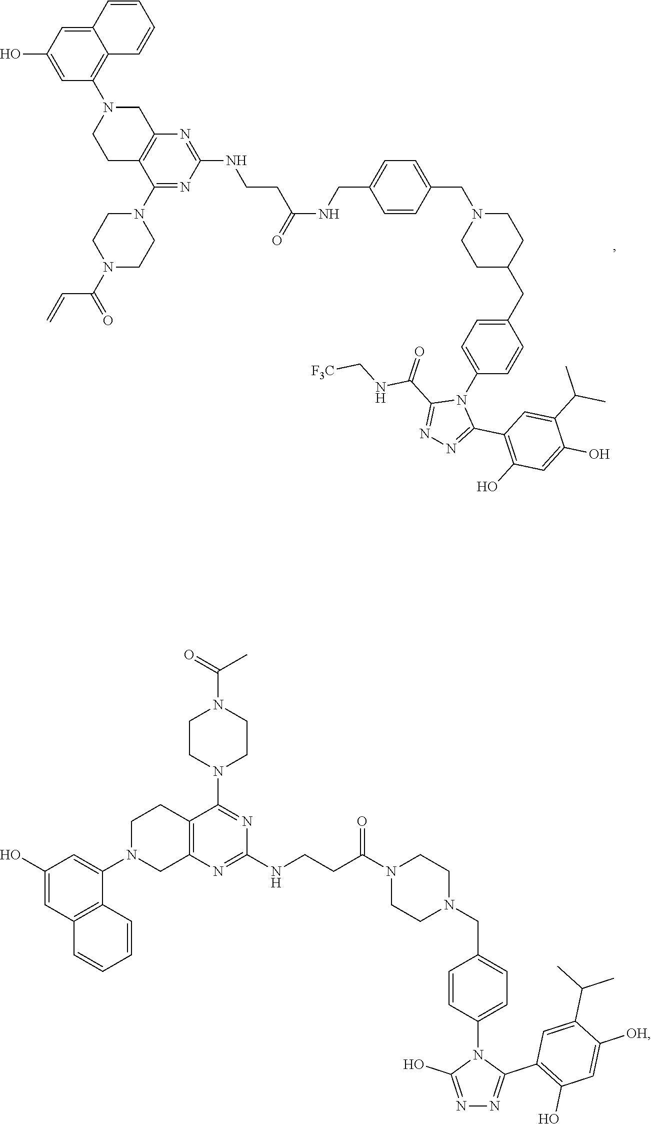 [SMILES: C=CC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; CC(=O)N1CCN(c2nc(NCCC(=O)N3CCN(Cc4ccc(-n5c(O)nnc5-c5cc(C(C)C)c(O)cc5O)cc4)CC3)nc3c2CCN(c2cc(O)cc4ccccc24)C3)CC1] 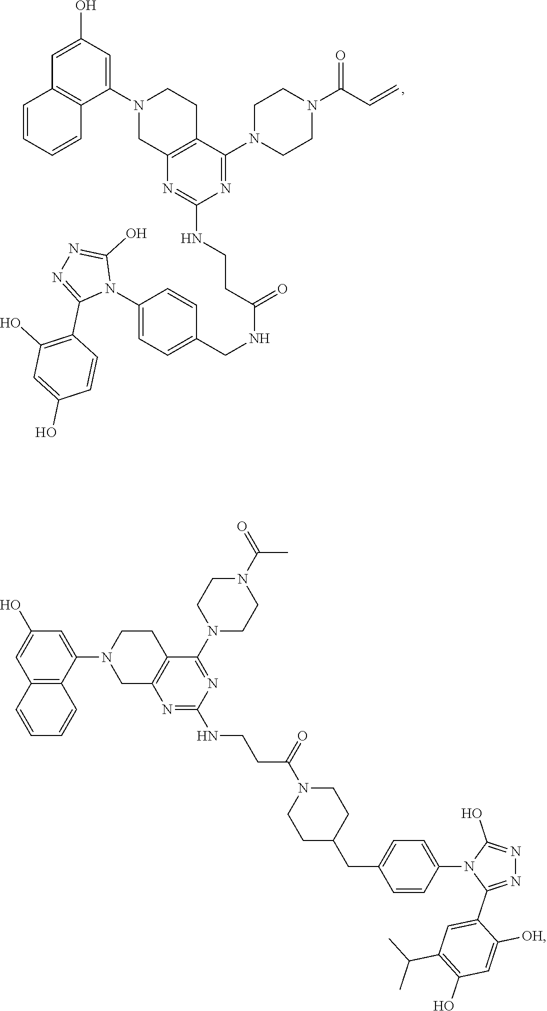 [SMILES: C=CC(=O)N1CCN(c2nc(NCCC(=O)NCc3ccc(-n4c(O)nnc4-c4ccc(O)cc4O)cc3)nc3c2CCN(c2cc(O)cc4ccccc24)C3)CC1 ; CC(=O)N1CCN(c2nc(NCCC(=O)N3CCC(Cc4ccc(-n5c(O)nnc5-c5cc(C(C)C)c(O)cc5O)cc4)CC3)nc3c2CCN(c2cc(O)cc4ccccc24)C3)CC1] 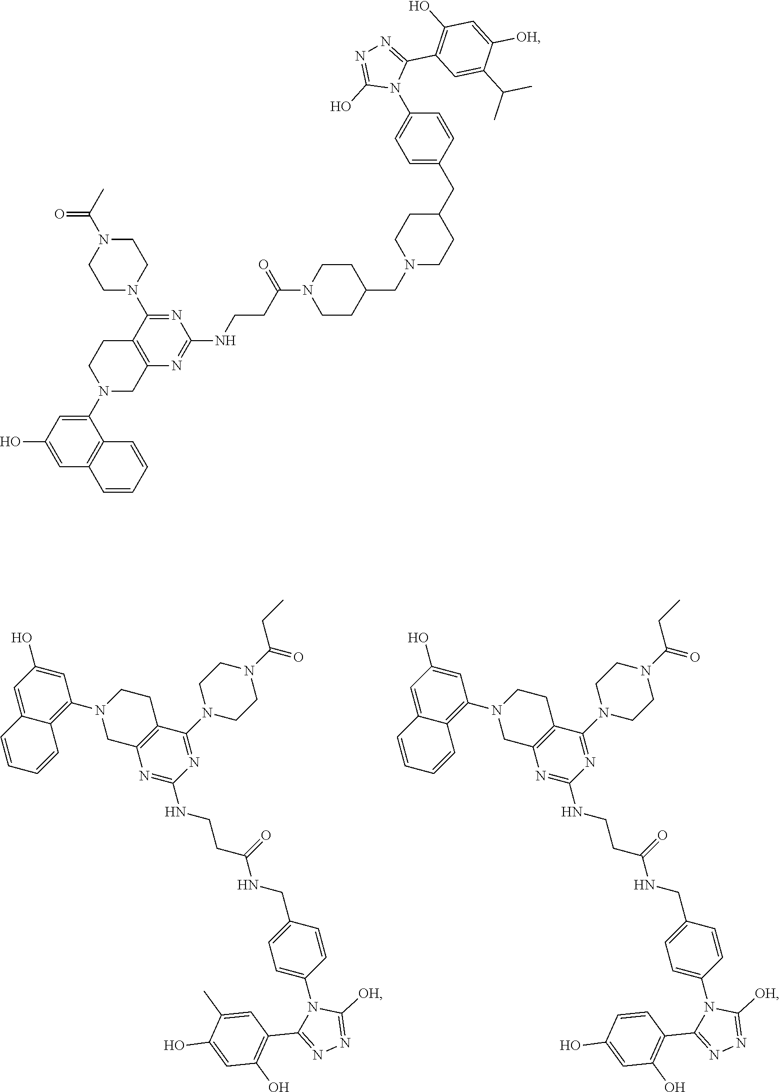 [SMILES: CC(=O)N1CCN(c2nc(NCCC(=O)N3CCC(CN4CCC(Cc5ccc(-n6c(O)nnc6-c6cc(C(C)C)c(O)cc6O)cc5)CC4)CC3)nc3c2CCN(c2cc(O)cc4ccccc24)C3)CC1 ; CCC(=O)N1CCN(c2nc(NCCC(=O)NCc3ccc(-n4c(O)nnc4-c4cc(C)c(O)cc4O)cc3)nc3c2CCN(c2cc(O)cc4ccccc24)C3)CC1 ; CCC(=O)N1CCN(c2nc(NCCC(=O)NCc3ccc(-n4c(O)nnc4-c4ccc(O)cc4O)cc3)nc3c2CCN(c2cc(O)cc4ccccc24)C3)CC1] 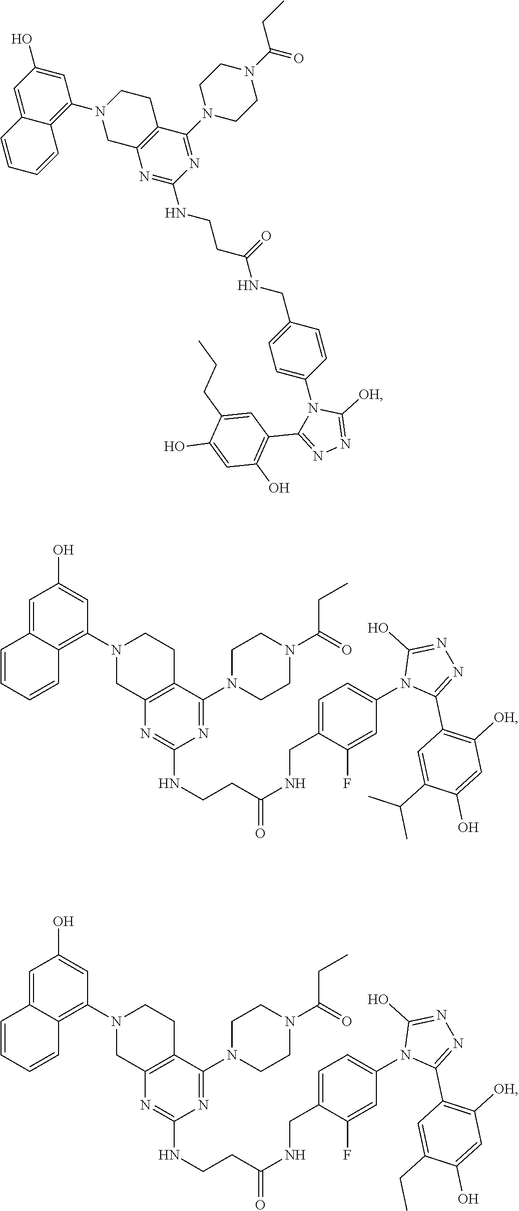 [SMILES: CCCc1cc(-c2nnc(O)n2-c2ccc(CNC(=O)CCNc3nc4c(c(N5CCN(C(=O)CC)CC5)n3)CCN(c3cc(O)cc5ccccc35)C4)cc2)c(O)cc1O ; CCC(=O)N1CCN(c2nc(NCCC(=O)NCc3ccc(-n4c(O)nnc4-c4cc(C(C)C)c(O)cc4O)cc3F)nc3c2CCN(c2cc(O)cc4ccccc24)C3)CC1 ; CCC(=O)N1CCN(c2nc(NCCC(=O)NCc3ccc(-n4c(O)nnc4-c4cc(CC)c(O)cc4O)cc3F)nc3c2CCN(c2cc(O)cc4ccccc24)C3)CC1] 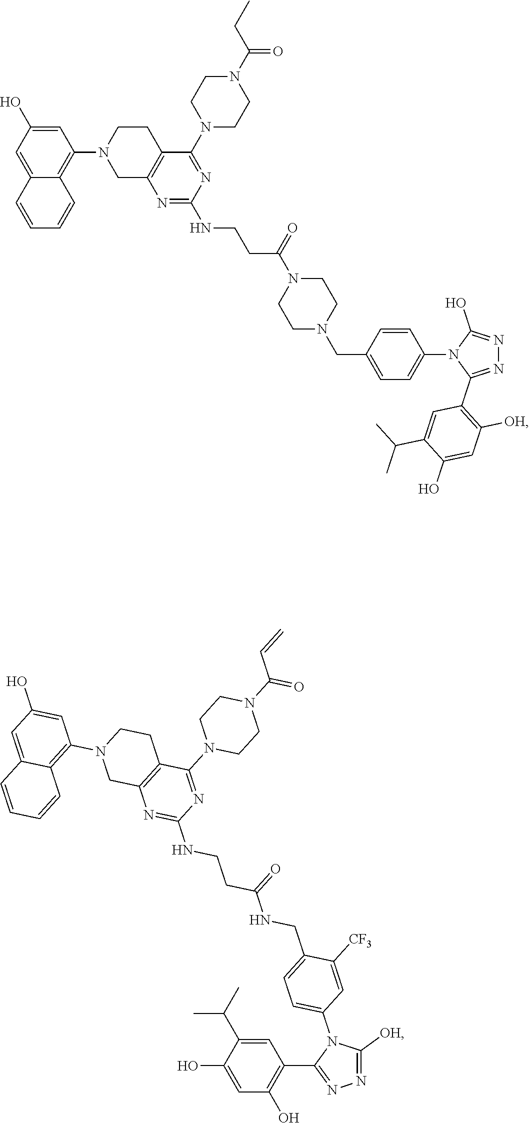 [SMILES: CCC(=O)N1CCN(c2nc(NCCC(=O)N3CCN(Cc4ccc(-n5c(O)nnc5-c5cc(C(C)C)c(O)cc5O)cc4)CC3)nc3c2CCN(c2cc(O)cc4ccccc24)C3)CC1 ; C=CC(=O)N1CCN(c2nc(NCCC(=O)NCc3ccc(-n4c(O)nnc4-c4cc(C(C)C)c(O)cc4O)cc3C(F)(F)F)nc3c2CCN(c2cc(O)cc4ccccc24)C3)CC1] 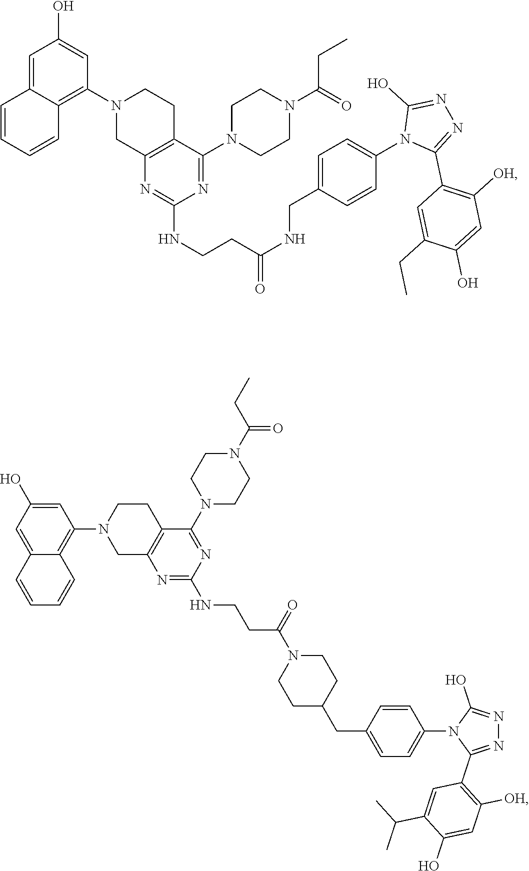 [SMILES: CCC(=O)N1CCN(c2nc(NCCC(=O)NCc3ccc(-n4c(O)nnc4-c4cc(CC)c(O)cc4O)cc3)nc3c2CCN(c2cc(O)cc4ccccc24)C3)CC1 ; CCC(=O)N1CCN(c2nc(NCCC(=O)N3CCC(Cc4ccc(-n5c(O)nnc5-c5cc(C(C)C)c(O)cc5O)cc4)CC3)nc3c2CCN(c2cc(O)cc4ccccc24)C3)CC1] 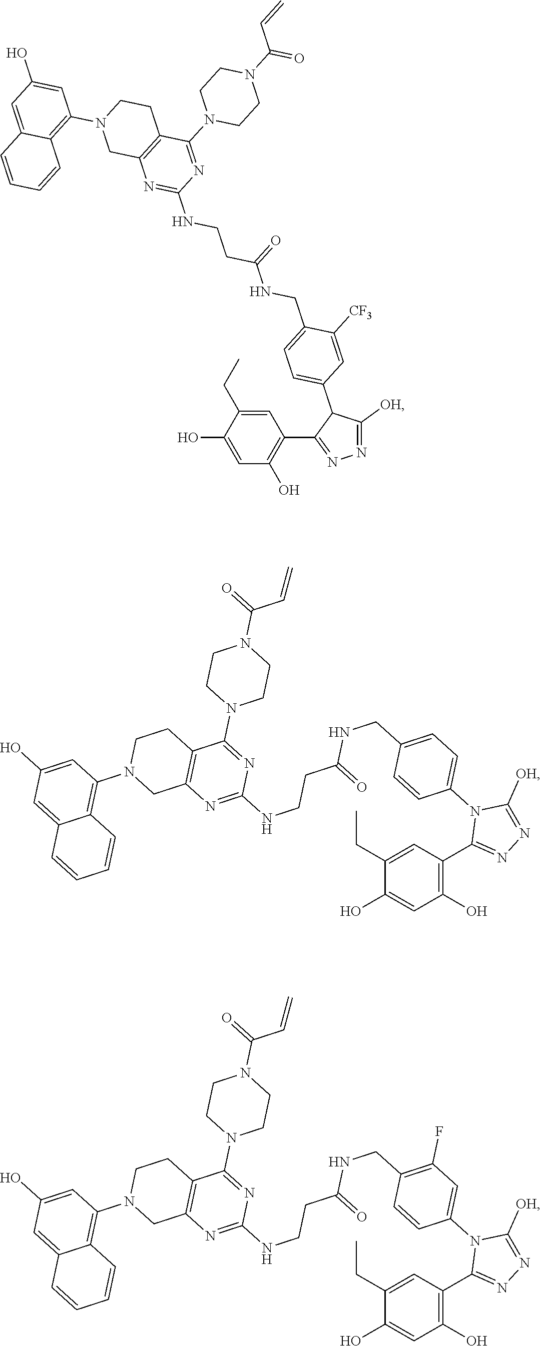 [SMILES: C=CC(=O)N1CCN(c2nc(NCCC(=O)NCc3ccc(C4C(O)=NN=C4c4cc(CC)c(O)cc4O)cc3C(F)(F)F)nc3c2CCN(c2cc(O)cc4ccccc24)C3)CC1 ; C=CC(=O)N1CCN(c2nc(NCCC(=O)NCc3ccc(-n4c(O)nnc4-c4cc(CC)c(O)cc4O)cc3)nc3c2CCN(c2cc(O)cc4ccccc24)C3)CC1 ; C=CC(=O)N1CCN(c2nc(NCCC(=O)NCc3ccc(-n4c(O)nnc4-c4cc(CC)c(O)cc4O)cc3F)nc3c2CCN(c2cc(O)cc4ccccc24)C3)CC1] 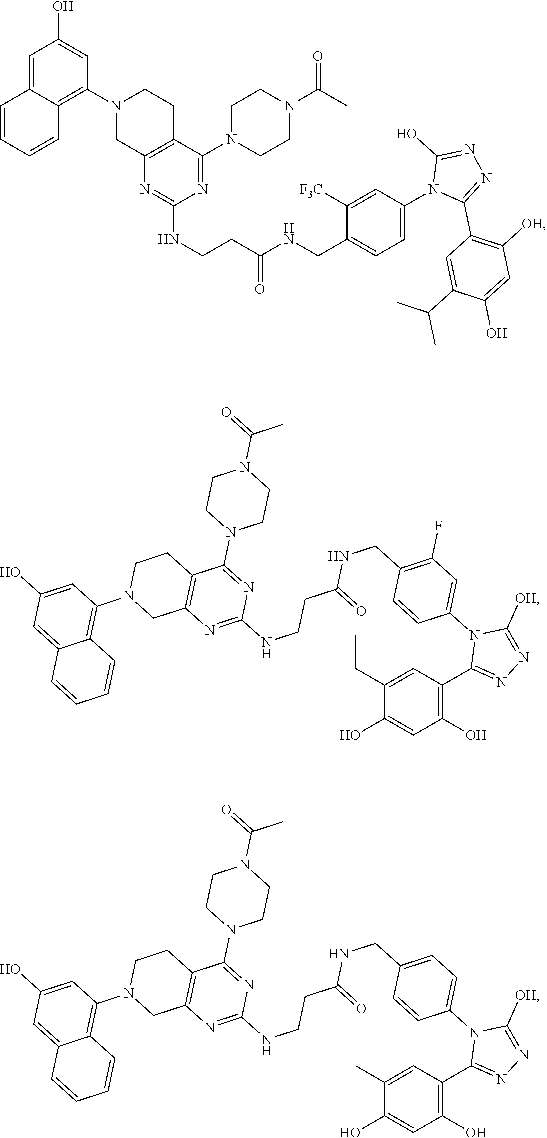 [SMILES: CC(=O)N1CCN(c2nc(NCCC(=O)NCc3ccc(-n4c(O)nnc4-c4cc(C(C)C)c(O)cc4O)cc3C(F)(F)F)nc3c2CCN(c2cc(O)cc4ccccc24)C3)CC1 ; CCc1cc(-c2nnc(O)n2-c2ccc(CNC(=O)CCNc3nc4c(c(N5CCN(C(C)=O)CC5)n3)CCN(c3cc(O)cc5ccccc35)C4)c(F)c2)c(O)cc1O ; CC(=O)N1CCN(c2nc(NCCC(=O)NCc3ccc(-n4c(O)nnc4-c4cc(C)c(O)cc4O)cc3)nc3c2CCN(c2cc(O)cc4ccccc24)C3)CC1] 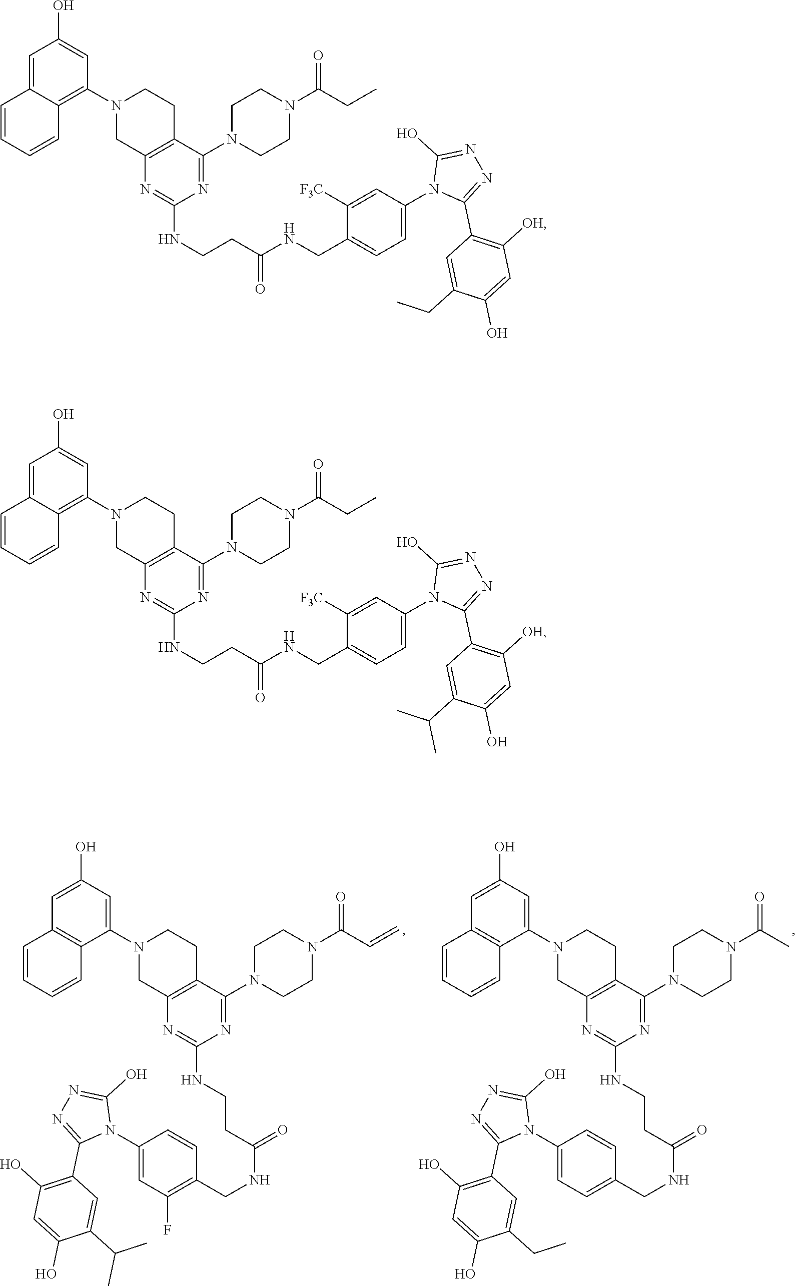 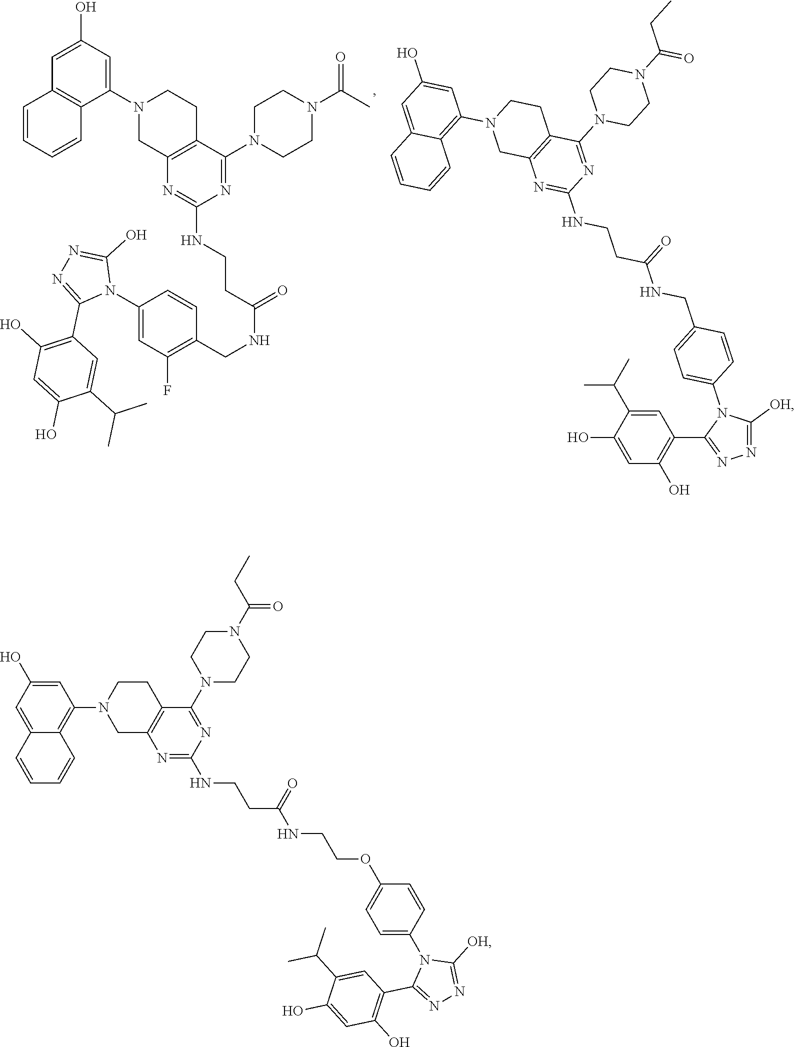 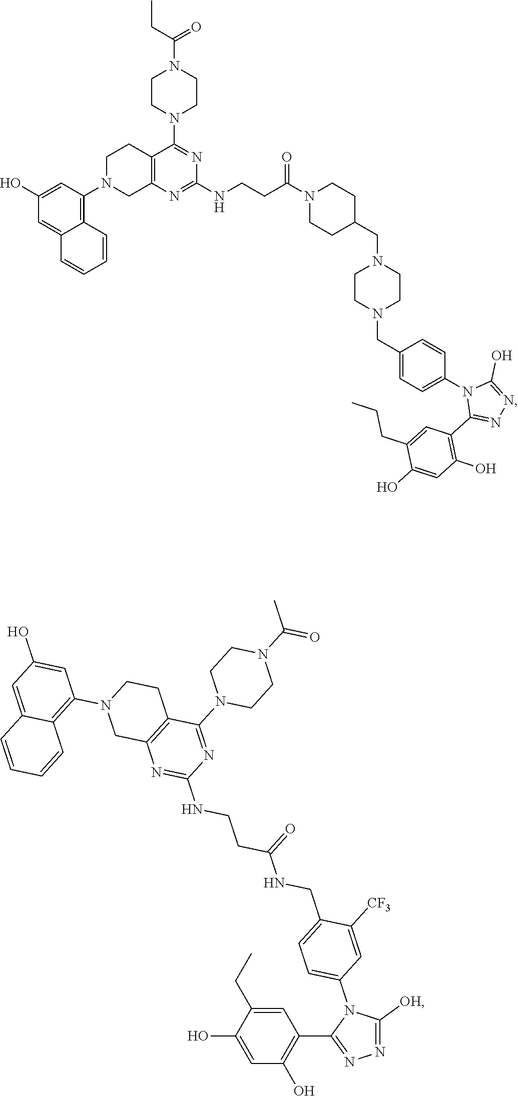 [SMILES: CCCc1cc(-c2nnc(O)n2-c2ccc(CN3CCN(CC4CCN(C(=O)CCNc5nc6c(c(N7CCN(C(=O)CC)CC7)n5)CCN(c5cc(O)cc7ccccc57)C6)CC4)CC3)cc2)c(O)cc1O ; CCc1cc(-c2nnc(O)n2-c2ccc(CNC(=O)CCNc3nc4c(c(N5CCN(C(C)=O)CC5)n3)CCN(c3cc(O)cc5ccccc35)C4)c(C(F)(F)F)c2)c(O)cc1O] 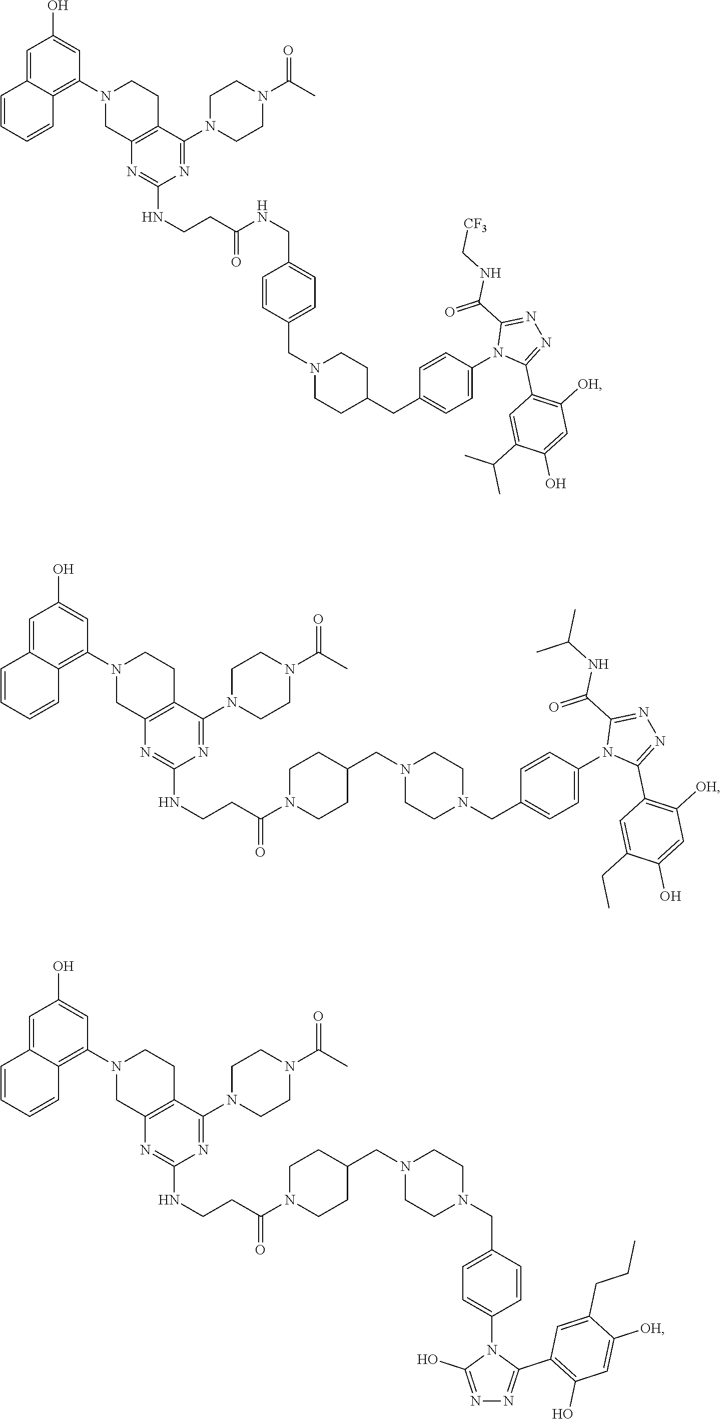 [SMILES: CC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; CCc1cc(-c2nnc(C(=O)NC(C)C)n2-c2ccc(CN3CCN(CC4CCN(C(=O)CCNc5nc6c(c(N7CCN(C(C)=O)CC7)n5)CCN(c5cc(O)cc7ccccc57)C6)CC4)CC3)cc2)c(O)cc1O ; CCCc1cc(-c2nnc(O)n2-c2ccc(CN3CCN(CC4CCN(C(=O)CCNc5nc6c(c(N7CCN(C(C)=O)CC7)n5)CCN(c5cc(O)cc7ccccc57)C6)CC4)CC3)cc2)c(O)cc1O] 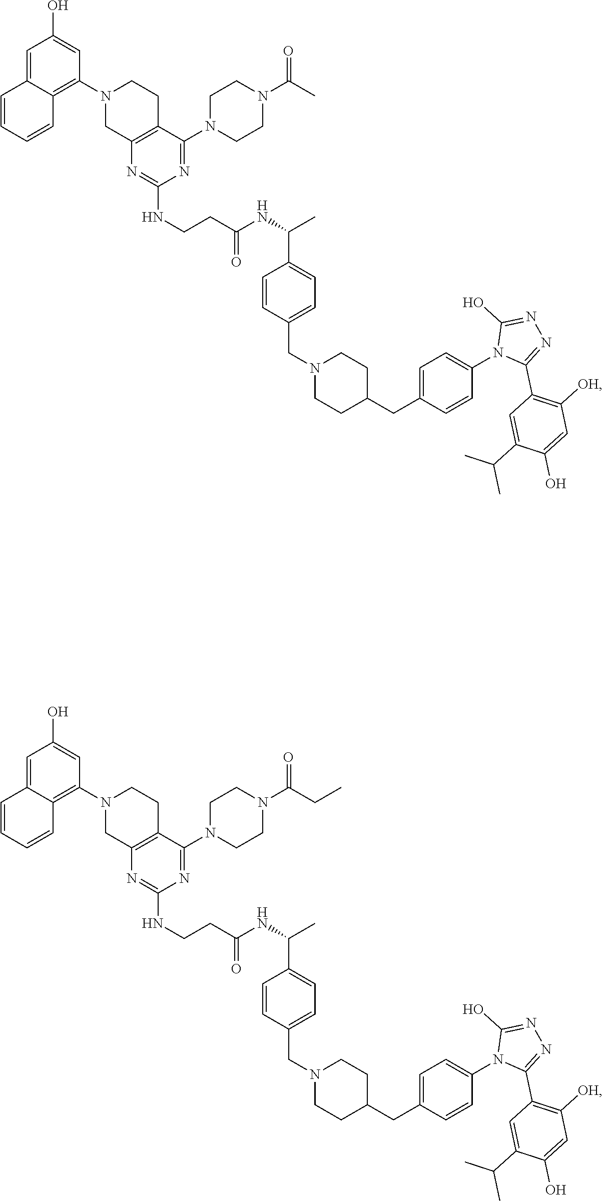 [SMILES: CC(=O)N1CCN(c2nc(NCCC(=O)N[C@H](C)c3ccc(CN4CCC(Cc5ccc(-n6c(O)nnc6-c6cc(C(C)C)c(O)cc6O)cc5)CC4)cc3)nc3c2CCN(c2cc(O)cc4ccccc24)C3)CC1 ; CCC(=O)N1CCN(c2nc(NCCC(=O)N[C@H](C)c3ccc(CN4CCC(Cc5ccc(-n6c(O)nnc6-c6cc(C(C)C)c(O)cc6O)cc5)CC4)cc3)nc3c2CCN(c2cc(O)cc4ccccc24)C3)CC1] 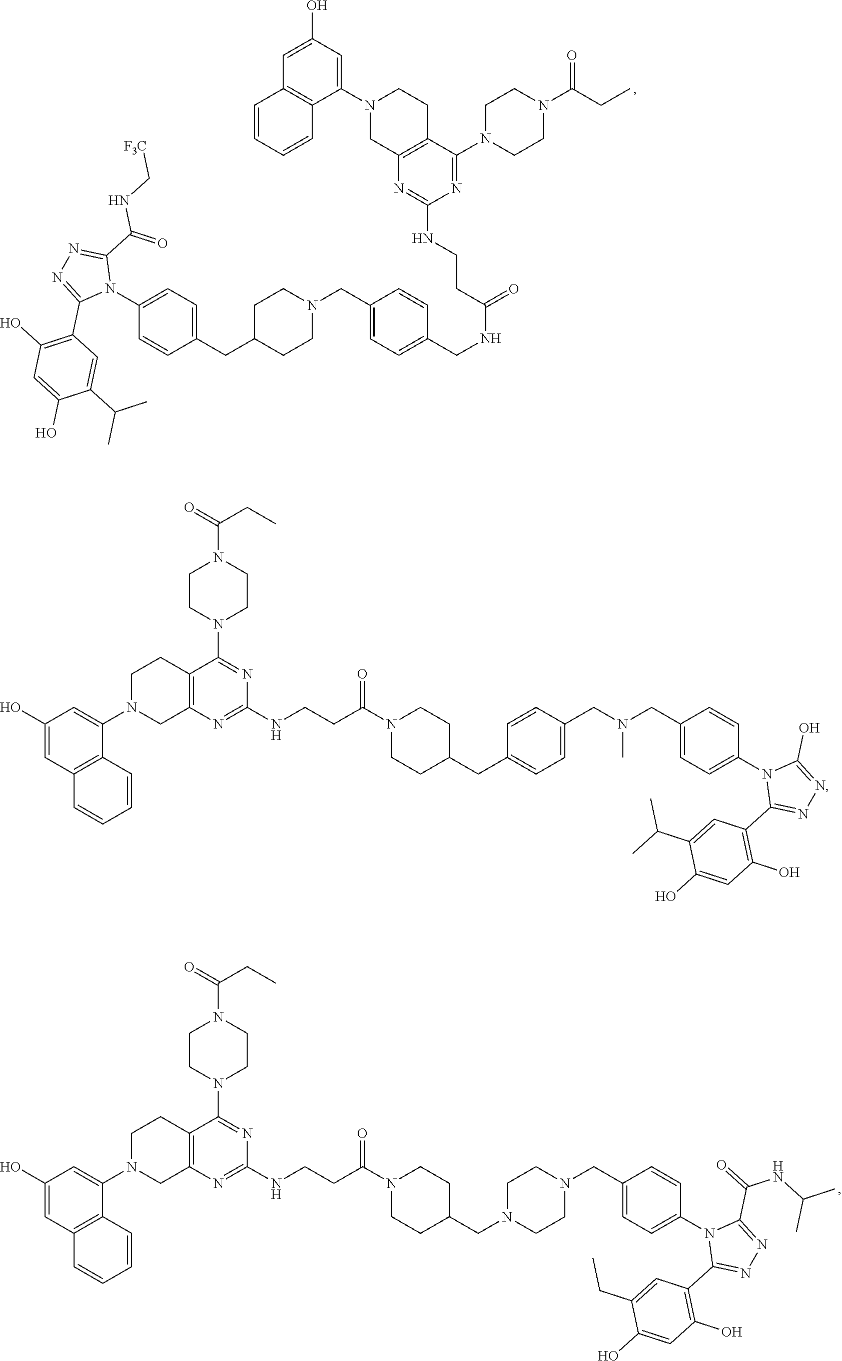 [SMILES: CCC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; CCC(=O)N1CCN(c2nc(NCCC(=O)N3CCC(Cc4ccc(CN(C)Cc5ccc(-n6c(O)nnc6-c6cc(C(C)C)c(O)cc6O)cc5)cc4)CC3)nc3c2CCN(c2cc(O)cc4ccccc24)C3)CC1 ; CCC(=O)N1CCN(c2nc(NCCC(=O)N3CCC(CN4CCN(Cc5ccc(-n6c(C(=O)NC(C)C)nnc6-c6cc(CC)c(O)cc6O)cc5)CC4)CC3)nc3c2CCN(c2cc(O)cc4ccccc24)C3)CC1] 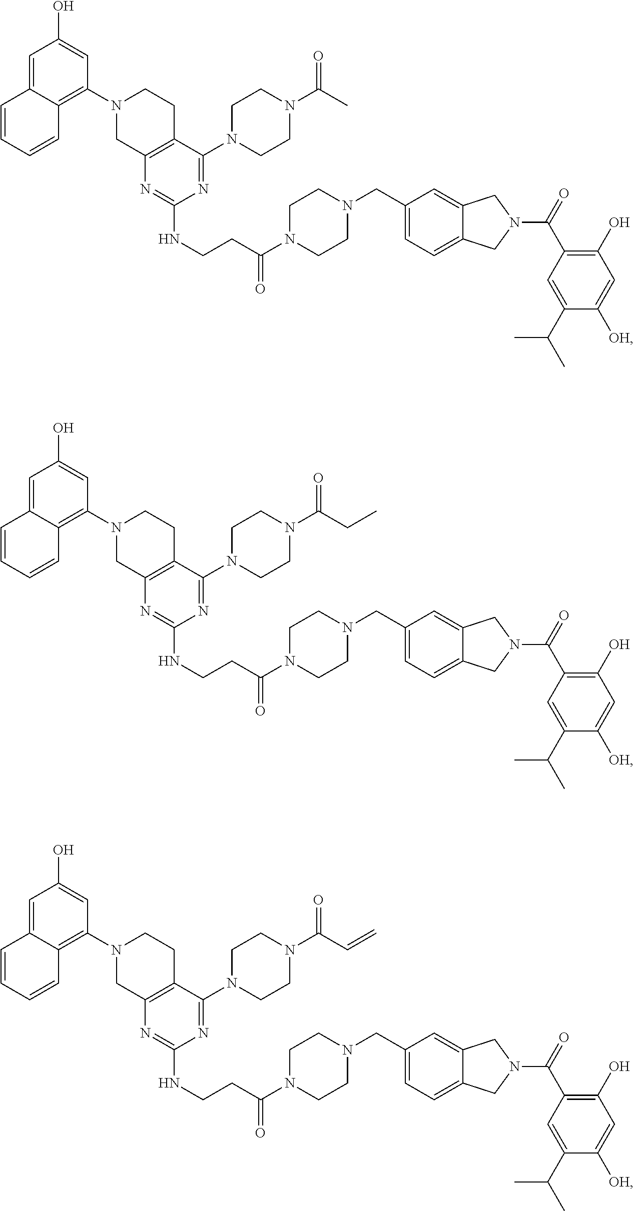 [SMILES: CC(=O)N1CCN(c2nc(NCCC(=O)N3CCN(Cc4ccc5c(c4)CN(C(=O)c4cc(C(C)C)c(O)cc4O)C5)CC3)nc3c2CCN(c2cc(O)cc4ccccc24)C3)CC1 ; CCC(=O)N1CCN(c2nc(NCCC(=O)N3CCN(Cc4ccc5c(c4)CN(C(=O)c4cc(C(C)C)c(O)cc4O)C5)CC3)nc3c2CCN(c2cc(O)cc4ccccc24)C3)CC1 ; C=CC(=O)N1CCN(c2nc(NCCC(=O)N3CCN(Cc4ccc5c(c4)CN(C(=O)c4cc(C(C)C)c(O)cc4O)C5)CC3)nc3c2CCN(c2cc(O)cc4ccccc24)C3)CC1] 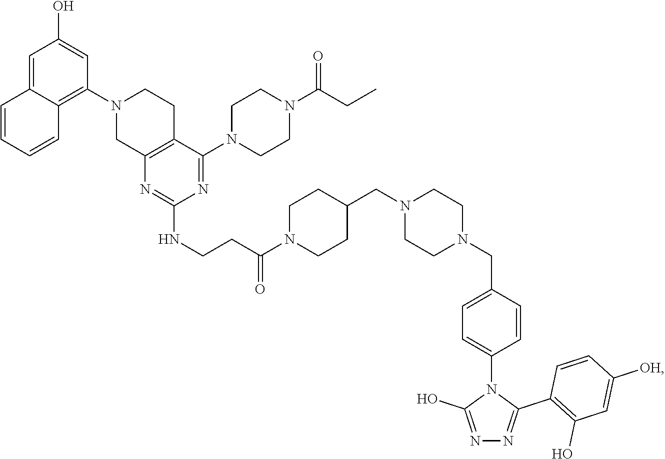 [SMILES: CCC(=O)N1CCN(c2nc(NCCC(=O)N3CCC(CN4CCN(Cc5ccc(-n6c(O)nnc6-c6ccc(O)cc6O)cc5)CC4)CC3)nc3c2CCN(c2cc(O)cc4ccccc24)C3)CC1] 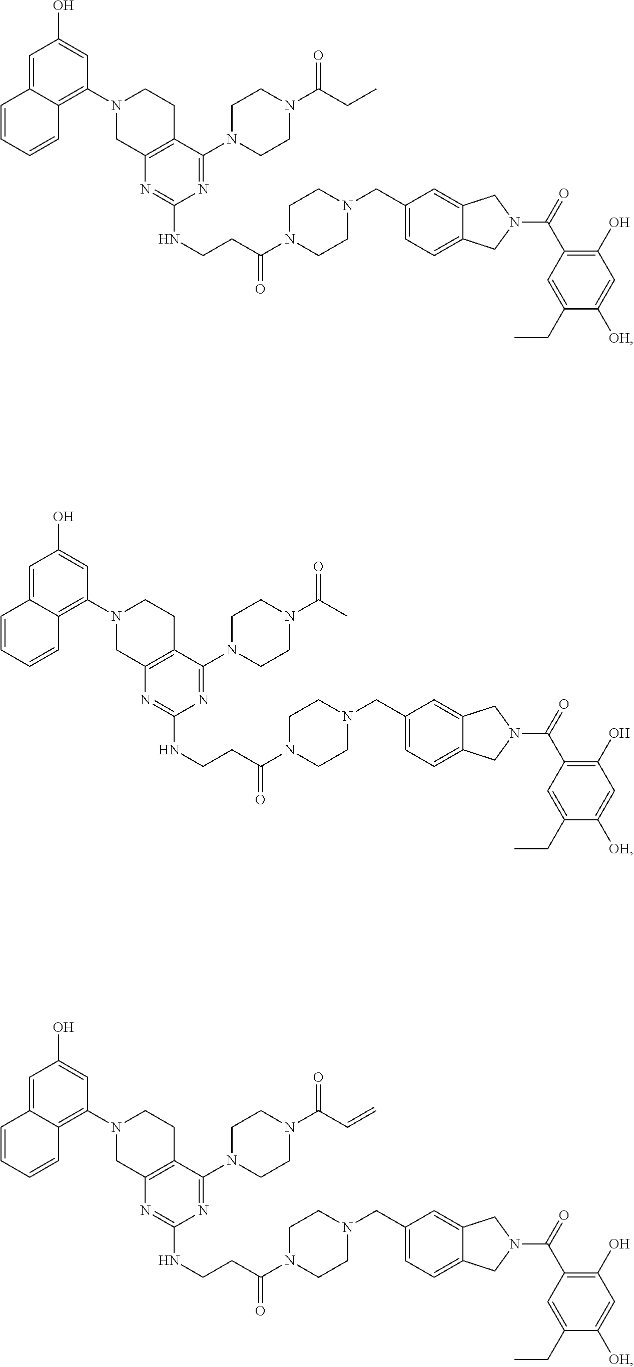 [SMILES: CCC(=O)N1CCN(c2nc(NCCC(=O)N3CCN(Cc4ccc5c(c4)CN(C(=O)c4cc(CC)c(O)cc4O)C5)CC3)nc3c2CCN(c2cc(O)cc4ccccc24)C3)CC1 ; CCc1cc(C(=O)N2Cc3ccc(CN4CCN(C(=O)CCNc5nc6c(c(N7CCN(C(C)=O)CC7)n5)CCN(c5cc(O)cc7ccccc57)C6)CC4)cc3C2)c(O)cc1O ; C=CC(=O)N1CCN(c2nc(NCCC(=O)N3CCN(Cc4ccc5c(c4)CN(C(=O)c4cc(CC)c(O)cc4O)C5)CC3)nc3c2CCN(c2cc(O)cc4ccccc24)C3)CC1] 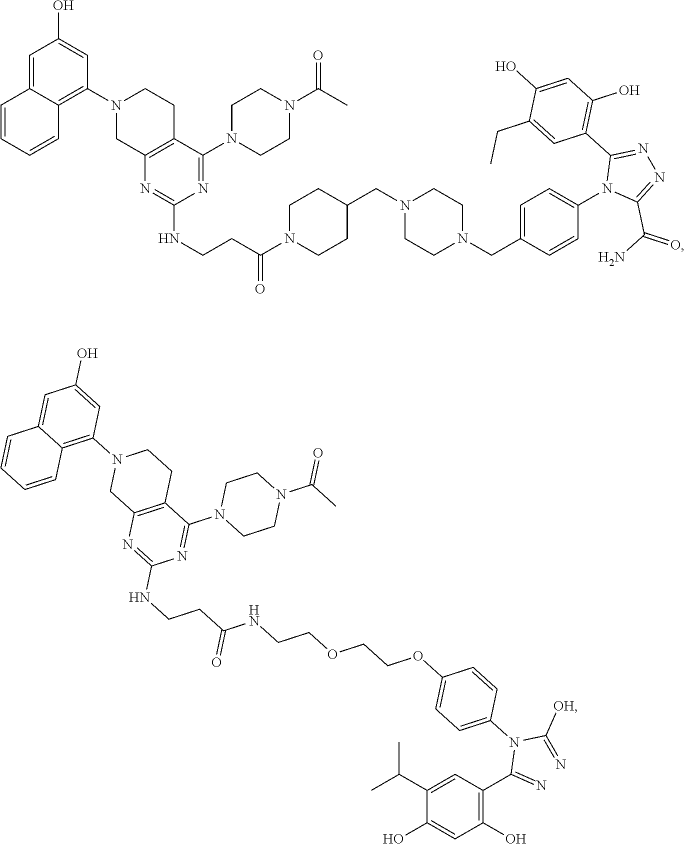 [SMILES: CCc1cc(-c2nnc(C(N)=O)n2-c2ccc(CN3CCN(CC4CCN(C(=O)CCNc5nc6c(c(N7CCN(C(C)=O)CC7)n5)CCN(c5cc(O)cc7ccccc57)C6)CC4)CC3)cc2)c(O)cc1O ; CC(=O)N1CCN(c2nc(NCCC(=O)NCCOCCOc3ccc(N(C(=N)O)C(=N)c4cc(C(C)C)c(O)cc4O)cc3)nc3c2CCN(c2cc(O)cc4ccccc24)C3)CC1] 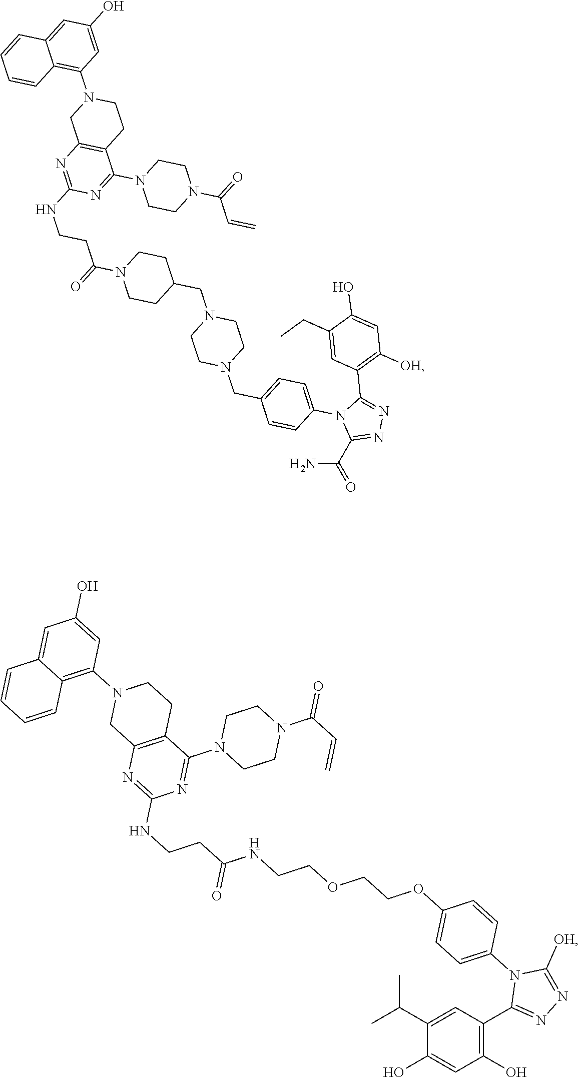 [SMILES: C=CC(=O)N1CCN(c2nc(NCCC(=O)N3CCC(CN4CCN(Cc5ccc(-n6c(C(N)=O)nnc6-c6cc(CC)c(O)cc6O)cc5)CC4)CC3)nc3c2CCN(c2cc(O)cc4ccccc24)C3)CC1 ; C=CC(=O)N1CCN(c2nc(NCCC(=O)NCCOCCOc3ccc(-n4c(O)nnc4-c4cc(C(C)C)c(O)cc4O)cc3)nc3c2CCN(c2cc(O)cc4ccccc24)C3)CC1] 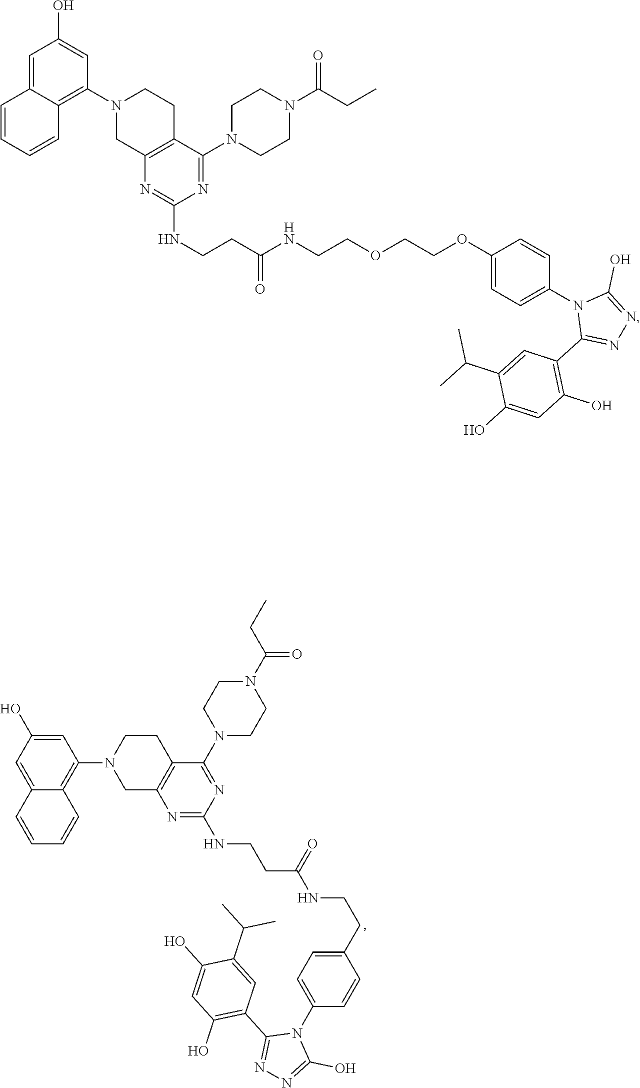 [SMILES: CCC(=O)N1CCN(c2nc(NCCC(=O)NCCOCCOc3ccc(-n4c(O)nnc4-c4cc(C(C)C)c(O)cc4O)cc3)nc3c2CCN(c2cc(O)cc4ccccc24)C3)CC1 ; CCC(=O)N1CCN(c2nc(NCCC(=O)NCCc3ccc(-n4c(O)nnc4-c4cc(C(C)C)c(O)cc4O)cc3)nc3c2CCN(c2cc(O)cc4ccccc24)C3)CC1] 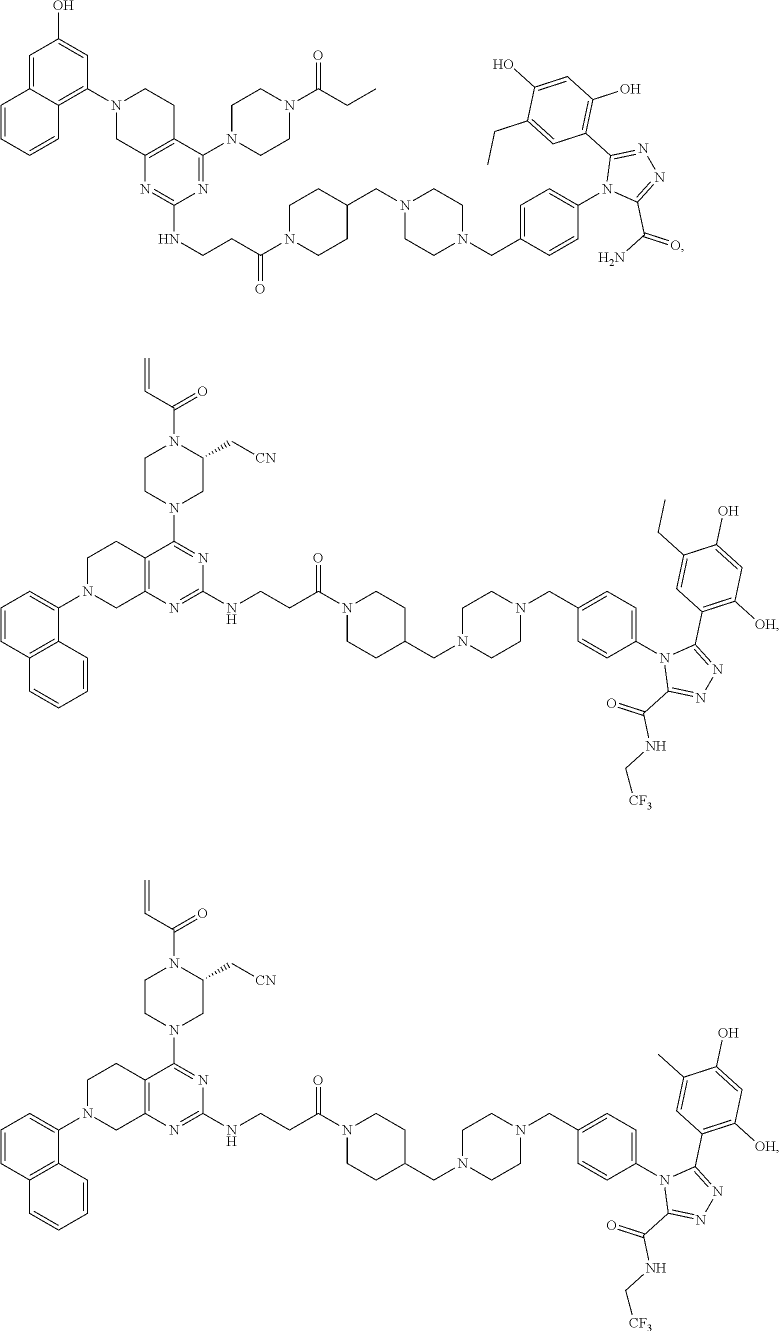 [SMILES: CCC(=O)N1CCN(c2nc(NCCC(=O)N3CCC(CN4CCN(Cc5ccc(-n6c(C(N)=O)nnc6-c6cc(CC)c(O)cc6O)cc5)CC4)CC3)nc3c2CCN(c2cc(O)cc4ccccc24)C3)CC1 ; C=CC(=O)N1CCN(c2nc(NCCC(=O)N3CCC(CN4CCN(Cc5ccc(-n6c(C(=O)NCC(F)(F)F)nnc6-c6cc(CC)c(O)cc6O)cc5)CC4)CC3)nc3c2CCN(c2cccc4ccccc24)C3)C[C@H]1CC#N ; C=CC(=O)N1CCN(c2nc(NCCC(=O)N3CCC(CN4CCN(Cc5ccc(-n6c(C(=O)NCC(F)(F)F)nnc6-c6cc(C)c(O)cc6O)cc5)CC4)CC3)nc3c2CCN(c2cccc4ccccc24)C3)C[C@H]1CC#N] 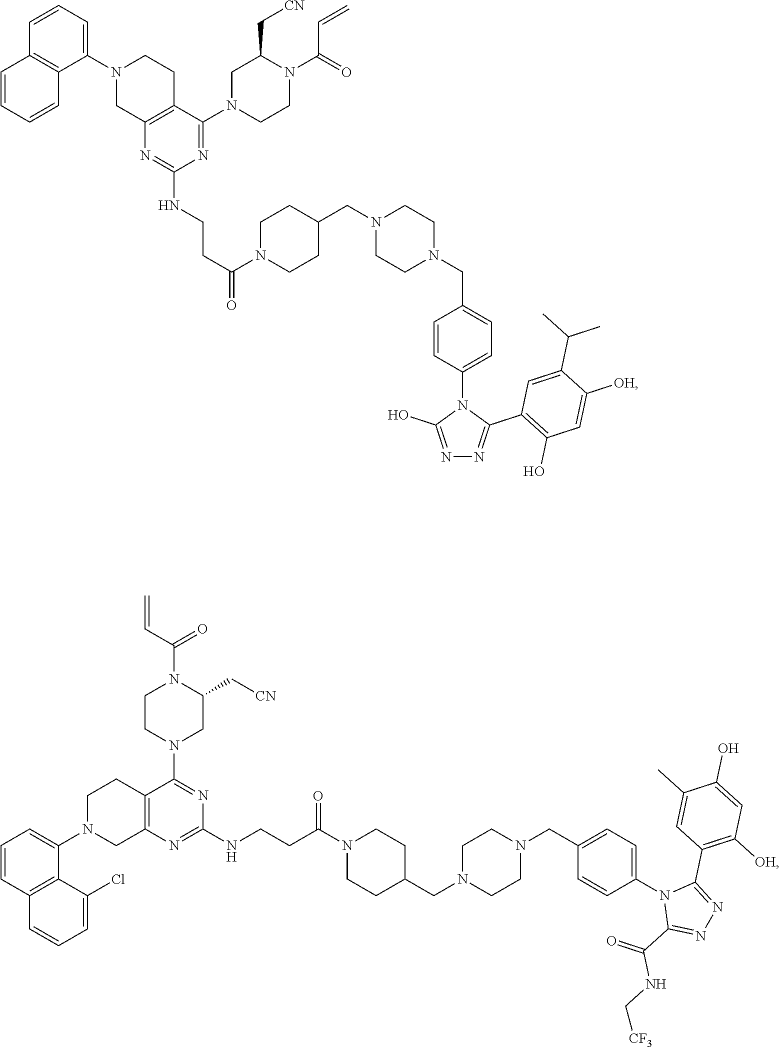 [SMILES: C=CC(=O)N1CCN(c2nc(NCCC(=O)N3CCC(CN4CCN(Cc5ccc(-n6c(O)nnc6-c6cc(C(C)C)c(O)cc6O)cc5)CC4)CC3)nc3c2CCN(c2cccc4ccccc24)C3)C[C@H]1CC#N ; C=CC(=O)N1CCN(c2nc(NCCC(=O)N3CCC(CN4CCN(Cc5ccc(-n6c(C(=O)NCC(F)(F)F)nnc6-c6cc(C)c(O)cc6O)cc5)CC4)CC3)nc3c2CCN(c2cccc4cccc(Cl)c24)C3)C[C@H]1CC#N] 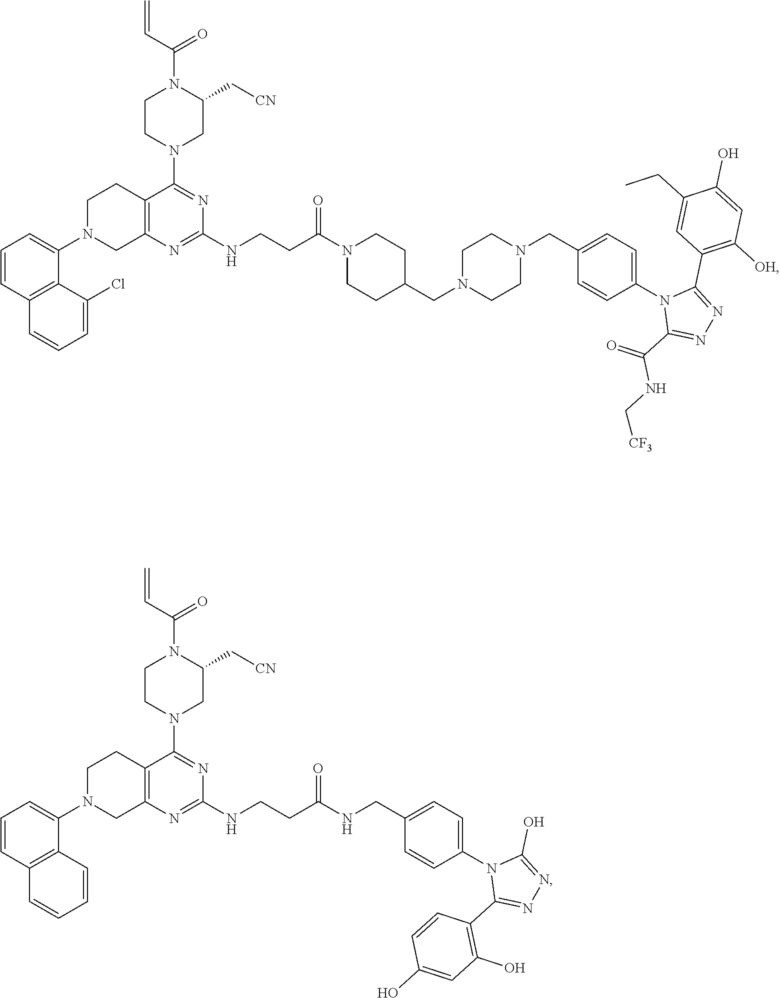 [SMILES: C=CC(=O)N1CCN(c2nc(NCCC(=O)N3CCC(CN4CCN(Cc5ccc(-n6c(C(=O)NCC(F)(F)F)nnc6-c6cc(CC)c(O)cc6O)cc5)CC4)CC3)nc3c2CCN(c2cccc4cccc(Cl)c24)C3)C[C@H]1CC#N ; C=CC(=O)N1CCN(c2nc(NCCC(=O)NCc3ccc(-n4c(O)nnc4-c4ccc(O)cc4O)cc3)nc3c2CCN(c2cccc4ccccc24)C3)C[C@H]1CC#N] 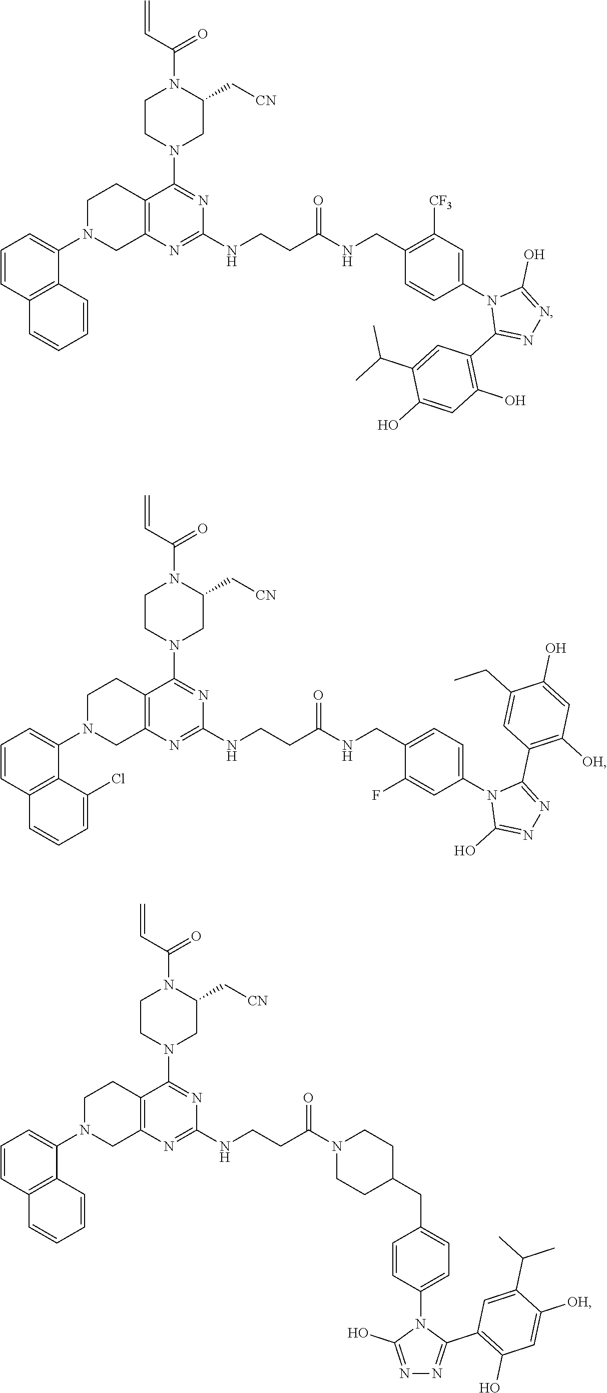 [SMILES: C=CC(=O)N1CCN(c2nc(NCCC(=O)NCc3ccc(-n4c(O)nnc4-c4cc(C(C)C)c(O)cc4O)cc3C(F)(F)F)nc3c2CCN(c2cccc4ccccc24)C3)C[C@H]1CC#N ; C=CC(=O)N1CCN(c2nc(NCCC(=O)NCc3ccc(-n4c(O)nnc4-c4cc(CC)c(O)cc4O)cc3F)nc3c2CCN(c2cccc4cccc(Cl)c24)C3)C[C@H]1CC#N ; C=CC(=O)N1CCN(c2nc(NCCC(=O)N3CCC(Cc4ccc(-n5c(O)nnc5-c5cc(C(C)C)c(O)cc5O)cc4)CC3)nc3c2CCN(c2cccc4ccccc24)C3)C[C@H]1CC#N] 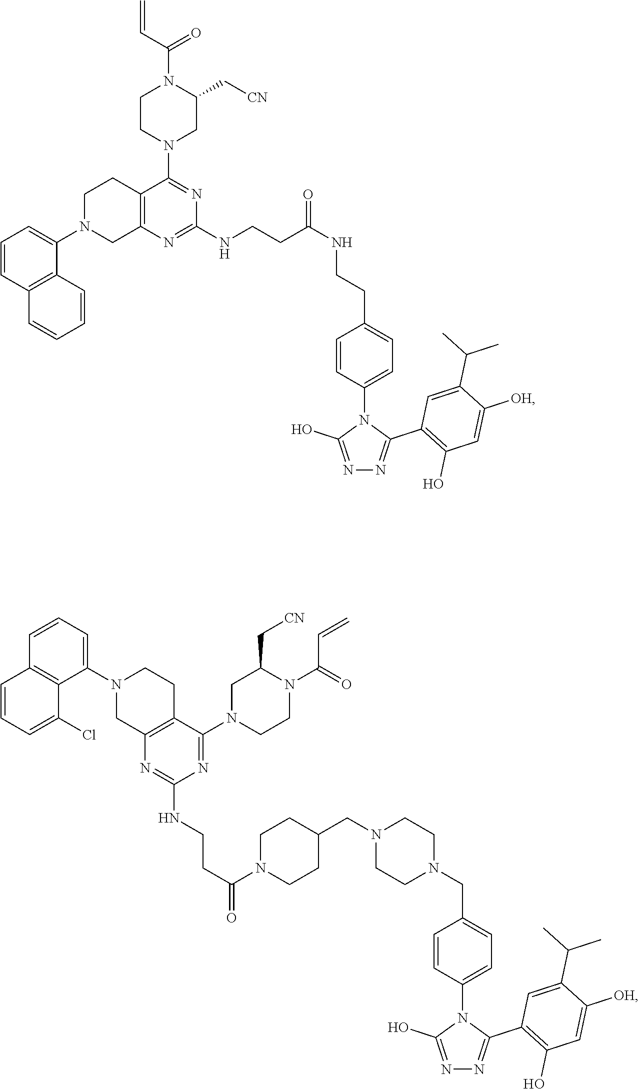 [SMILES: C=CC(=O)N1CCN(c2nc(NCCC(=O)NCCc3ccc(-n4c(O)nnc4-c4cc(C(C)C)c(O)cc4O)cc3)nc3c2CCN(c2cccc4ccccc24)C3)C[C@H]1CC#N ; C=CC(=O)N1CCN(c2nc(NCCC(=O)N3CCC(CN4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@H]1CC#N] 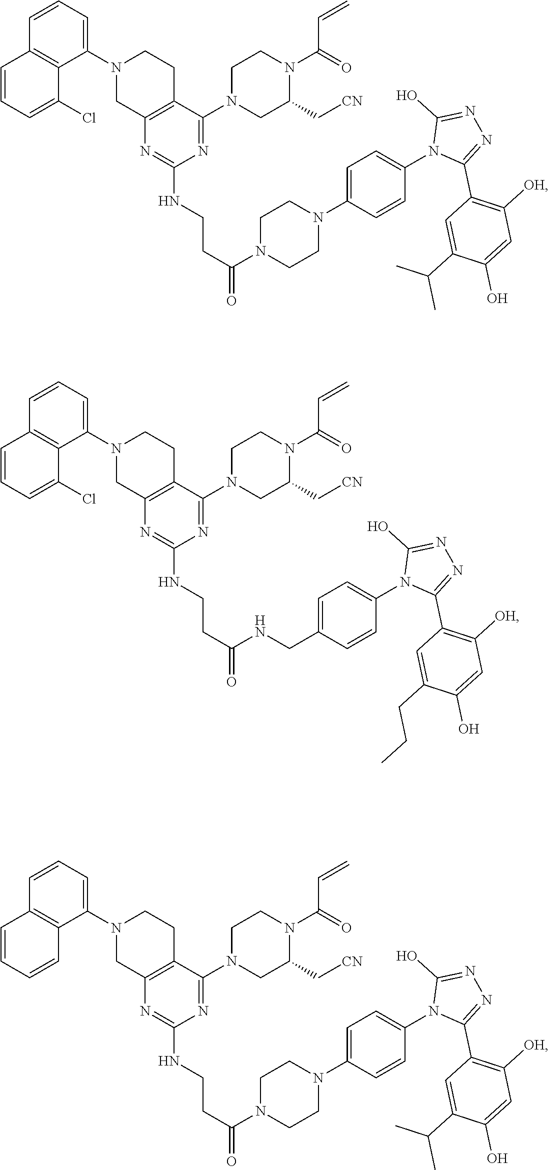 [SMILES: C=CC(=O)N1CCN(c2nc(NCCC(=O)N3CCN(c4ccc(-n5c(O)nnc5-c5cc(C(C)C)c(O)cc5O)cc4)CC3)nc3c2CCN(c2cccc4cccc(Cl)c24)C3)C[C@H]1CC#N ; C=CC(=O)N1CCN(c2nc(NCCC(=O)NCc3ccc(-n4c(O)nnc4-c4cc(CCC)c(O)cc4O)cc3)nc3c2CCN(c2cccc4cccc(Cl)c24)C3)C[C@H]1CC#N ; C=CC(=O)N1CCN(c2nc(NCCC(=O)N3CCN(c4ccc(-n5c(O)nnc5-c5cc(C(C)C)c(O)cc5O)cc4)CC3)nc3c2CCN(c2cccc4ccccc24)C3)C[C@H]1CC#N] 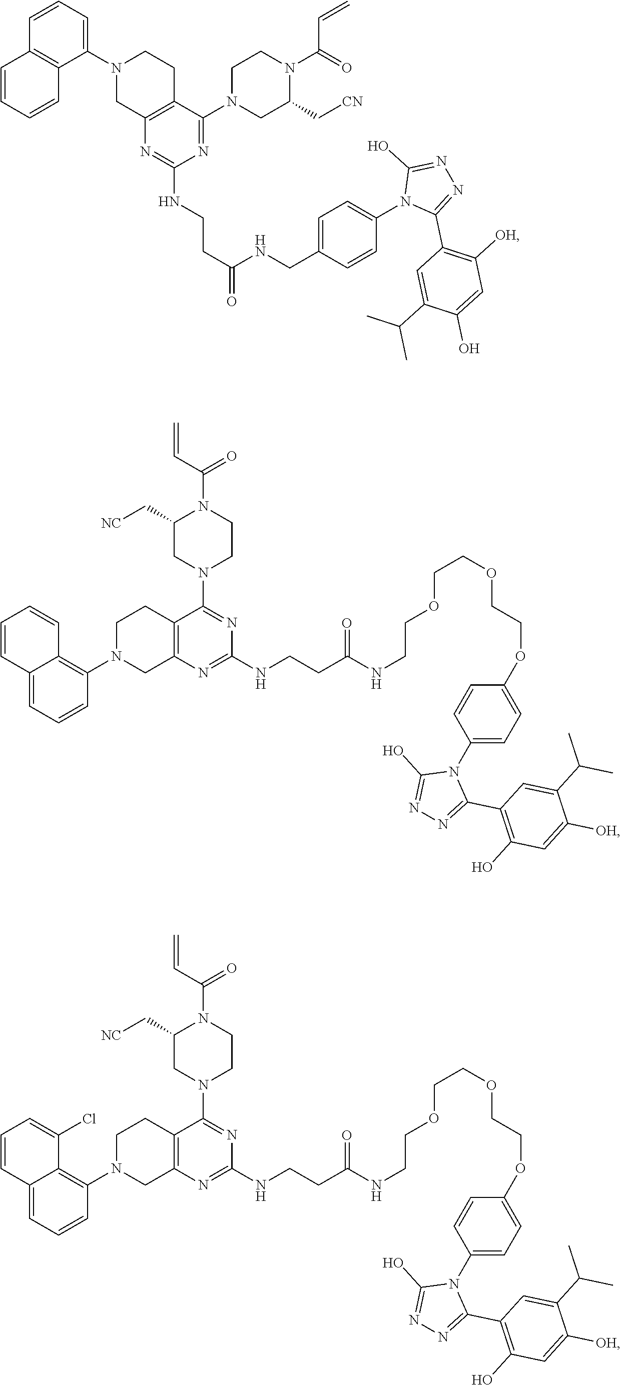 [SMILES: C=CC(=O)N1CCN(c2nc(NCCC(=O)NCc3ccc(-n4c(O)nnc4-c4cc(C(C)C)c(O)cc4O)cc3)nc3c2CCN(c2cccc4ccccc24)C3)C[C@H]1CC#N ; C=CC(=O)N1CCN(c2nc(NCCC(=O)NCCOCCOCCOc3ccc(-n4c(O)nnc4-c4cc(C(C)C)c(O)cc4O)cc3)nc3c2CCN(c2cccc4ccccc24)C3)C[C@@H]1CC#N ; C=CC(=O)N1CCN(c2nc(NCCC(=O)NCCOCCOCCOc3ccc(-n4c(O)nnc4-c4cc(C(C)C)c(O)cc4O)cc3)nc3c2CCN(c2cccc4cccc(Cl)c24)C3)C[C@@H]1CC#N] 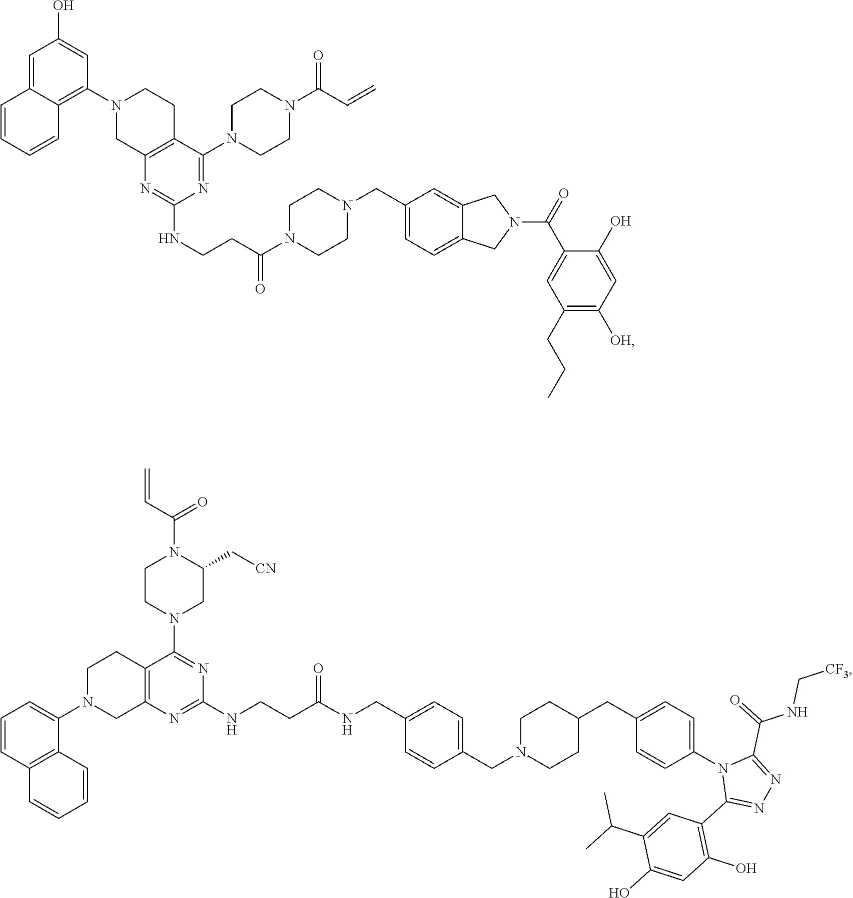 [SMILES: C=CC(=O)N1CCN(c2nc(NCCC(=O)N3CCN(Cc4ccc5c(c4)CN(C(=O)c4cc(CCC)c(O)cc4O)C5)CC3)nc3c2CCN(c2cc(O)cc4ccccc24)C3)CC1 ; C=CC(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cccc4ccccc24)C3)C[C@H]1CC#N] 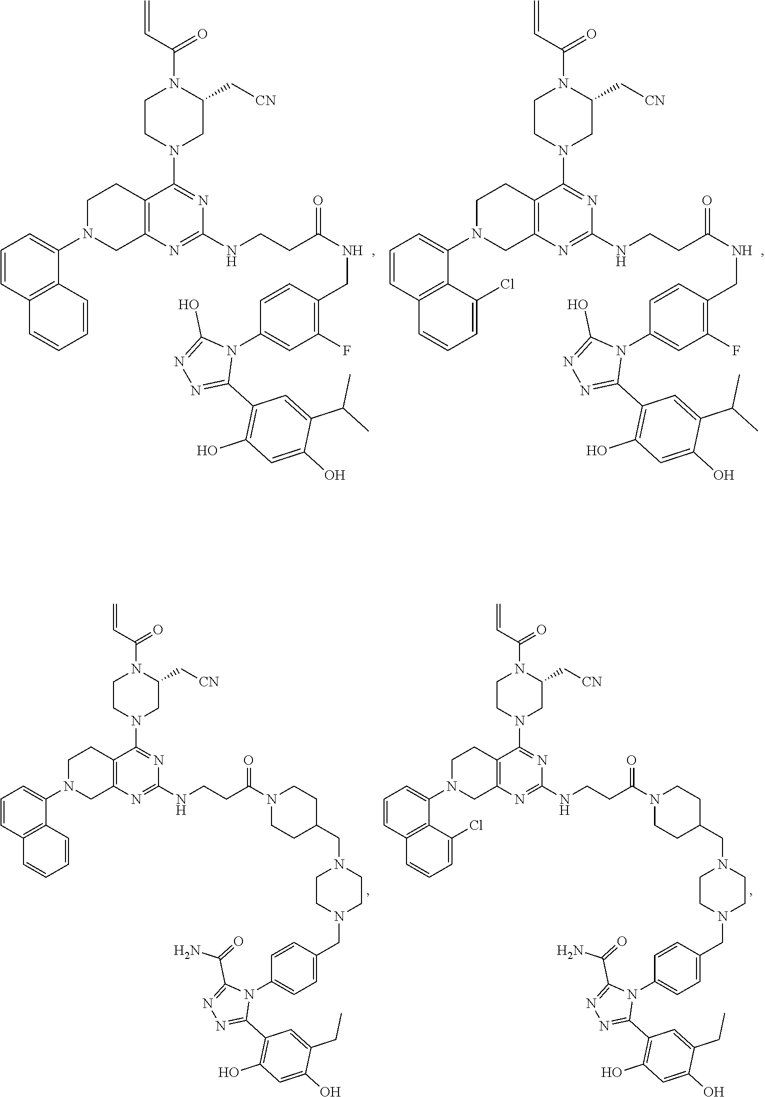 [SMILES: C=CC(=O)N1CCN(c2nc(NCCC(=O)NCc3ccc(-n4c(O)nnc4-c4cc(C(C)C)c(O)cc4O)cc3F)nc3c2CCN(c2cccc4ccccc24)C3)C[C@H]1CC#N ; C=CC(=O)N1CCN(c2nc(NCCC(=O)NCc3ccc(-n4c(O)nnc4-c4cc(C(C)C)c(O)cc4O)cc3F)nc3c2CCN(c2cccc4cccc(Cl)c24)C3)C[C@H]1CC#N ; C=CC(=O)N1CCN(c2nc(NCCC(=O)N3CCC(CN4CCN(Cc5ccc(-n6c(C(N)=O)nnc6-c6cc(CC)c(O)cc6O)cc5)CC4)CC3)nc3c2CCN(c2cccc4ccccc24)C3)C[C@H]1CC#N ; C=CC(=O)N1CCN(c2nc(NCCC(=O)N3CCC(CN4CCN(Cc5ccc(-n6c(C(N)=O)nnc6-c6cc(CC)c(O)cc6O)cc5)CC4)CC3)nc3c2CCN(c2cccc4cccc(Cl)c24)C3)C[C@H]1CC#N] 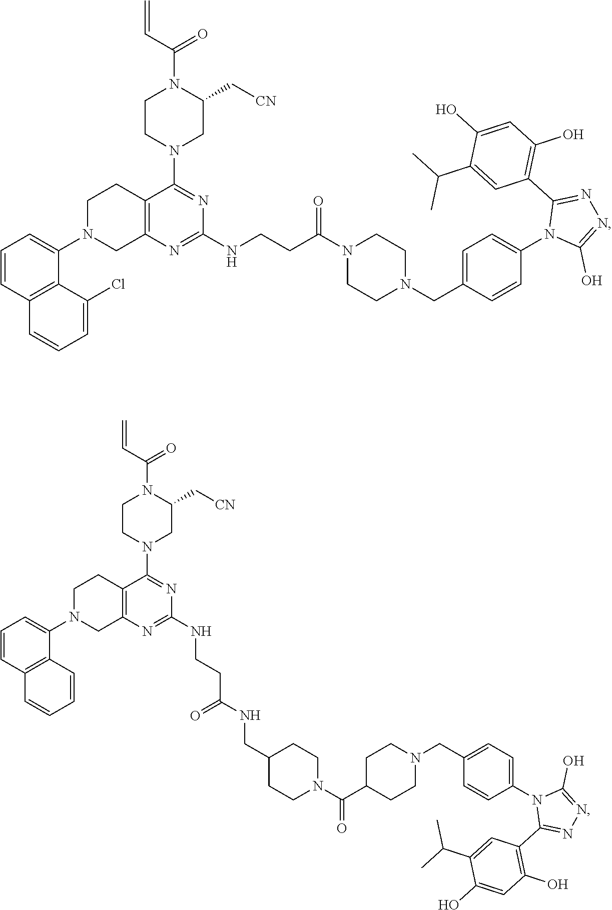 [SMILES: C=CC(=O)N1CCN(c2nc(NCCC(=O)N3CCN(Cc4ccc(-n5c(O)nnc5-c5cc(C(C)C)c(O)cc5O)cc4)CC3)nc3c2CCN(c2cccc4cccc(Cl)c24)C3)C[C@H]1CC#N ; C=CC(=O)N1CCN(c2nc(NCCC(=O)NCC3CCN(C(=O)C4CCN(Cc5ccc(-n6c(O)nnc6-c6cc(C(C)C)c(O)cc6O)cc5)CC4)CC3)nc3c2CCN(c2cccc4ccccc24)C3)C[C@H]1CC#N] 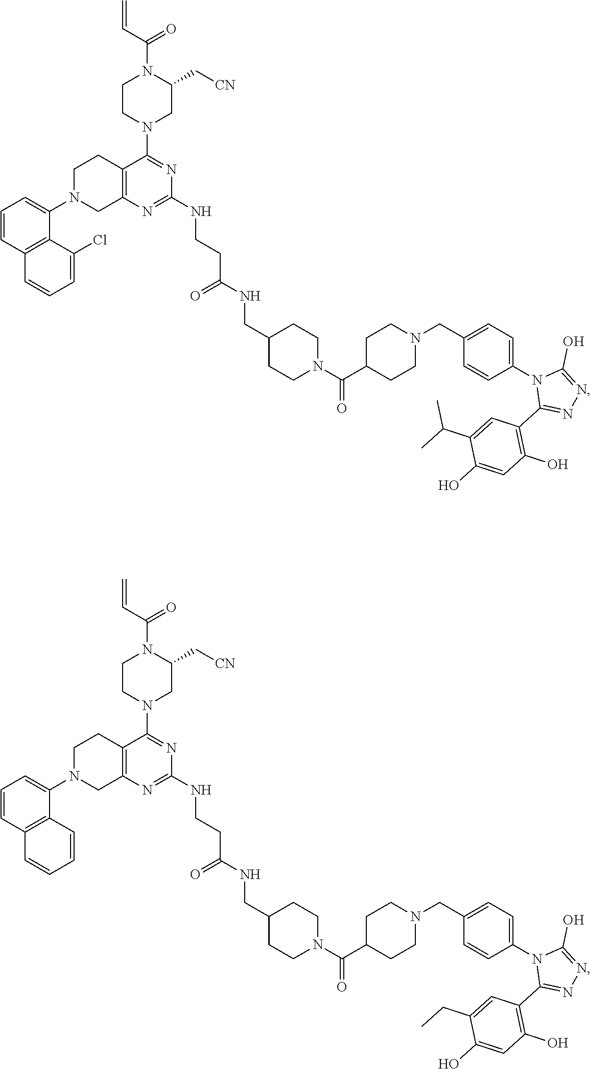 [SMILES: C=CC(=O)N1CCN(c2nc(NCCC(=O)NCC3CCN(C(=O)C4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@H]1CC#N ; C=CC(=O)N1CCN(c2nc(NCCC(=O)NCC3CCN(C(=O)C4CCN(Cc5ccc(-n6c(O)nnc6-c6cc(CC)c(O)cc6O)cc5)CC4)CC3)nc3c2CCN(c2cccc4ccccc24)C3)C[C@H]1CC#N] 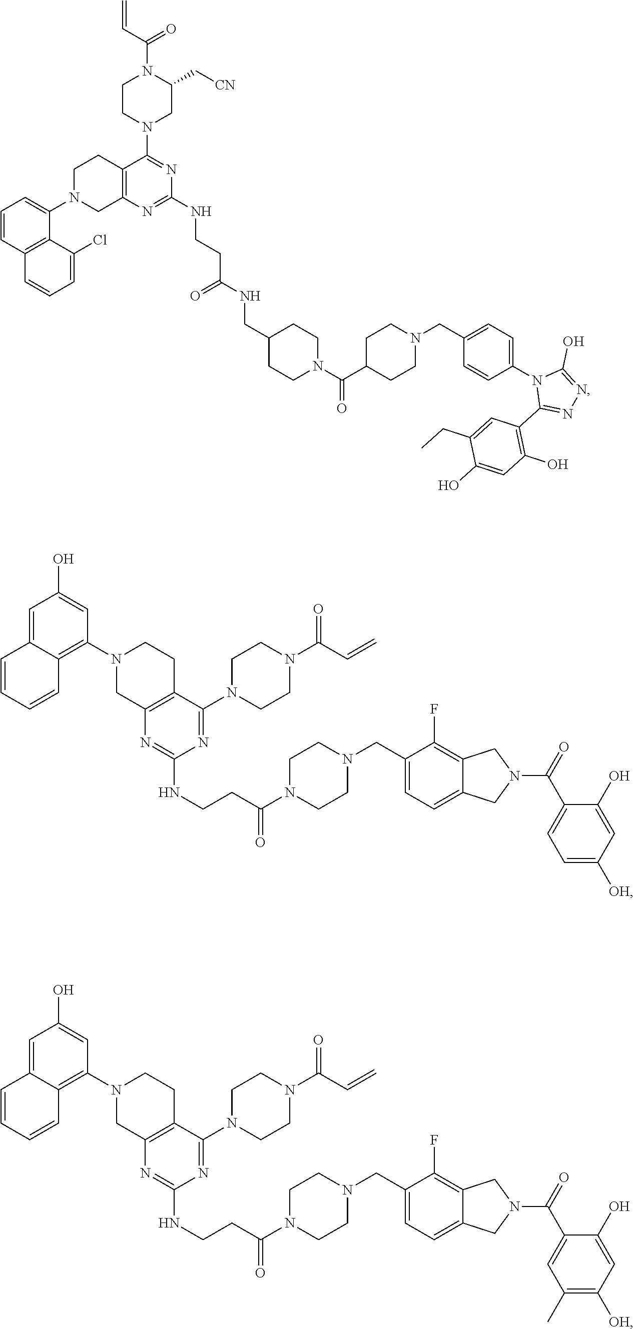 [SMILES: C=CC(=O)N1CCN(c2nc(NCCC(=O)NCC3CCN(C(=O)C4CCN(Cc5ccc(-n6c(O)nnc6-c6cc(CC)c(O)cc6O)cc5)CC4)CC3)nc3c2CCN(c2cccc4cccc(Cl)c24)C3)C[C@H]1CC#N ; C=CC(=O)N1CCN(c2nc(NCCC(=O)N3CCN(Cc4ccc5c(c4F)CN(C(=O)c4ccc(O)cc4O)C5)CC3)nc3c2CCN(c2cc(O)cc4ccccc24)C3)CC1 ; C=CC(=O)N1CCN(c2nc(NCCC(=O)N3CCN(Cc4ccc5c(c4F)CN(C(=O)c4cc(C)c(O)cc4O)C5)CC3)nc3c2CCN(c2cc(O)cc4ccccc24)C3)CC1] 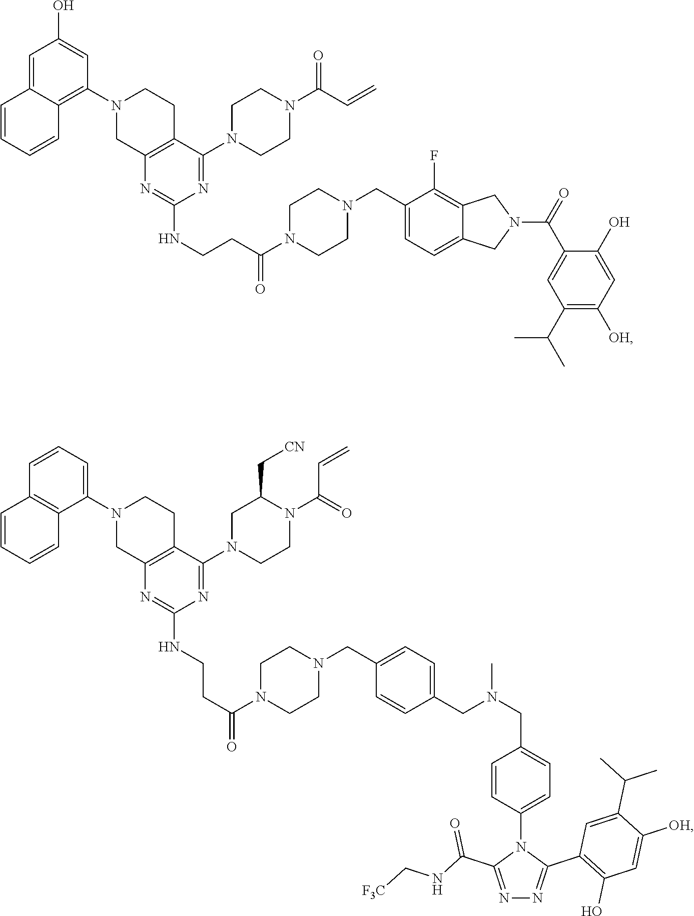 [SMILES: C=CC(=O)N1CCN(c2nc(NCCC(=O)N3CCN(Cc4ccc5c(c4F)CN(C(=O)c4cc(C(C)C)c(O)cc4O)C5)CC3)nc3c2CCN(c2cc(O)cc4ccccc24)C3)CC1 ; C=CC(=O)N1CCN(c2nc(NCCC(=O)N3CCN(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cccc4ccccc24)C3)C[C@H]1CC#N] 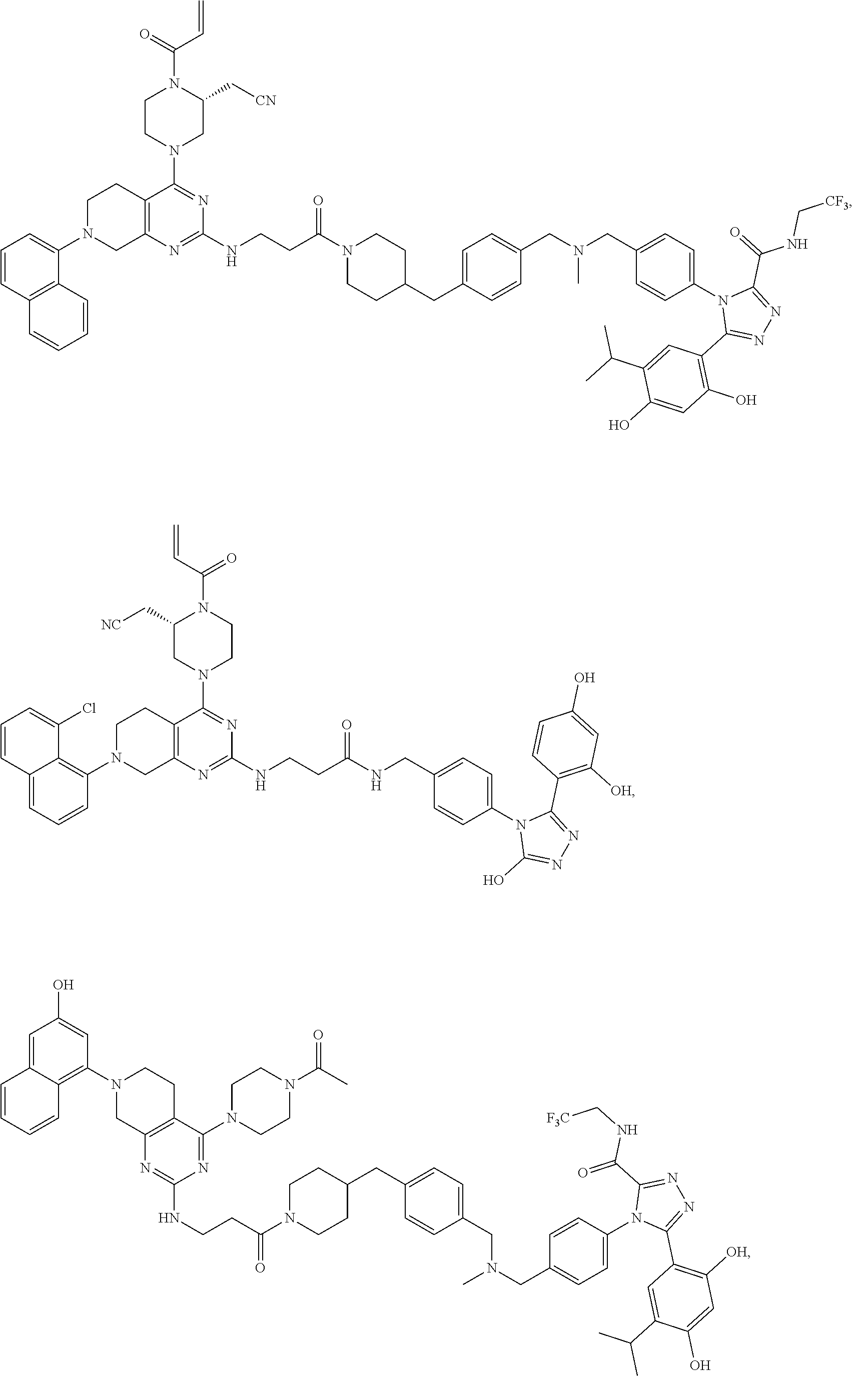 [SMILES: C=CC(=O)N1CCN(c2nc(NCCC(=O)N3CCC(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cccc4ccccc24)C3)C[C@H]1CC#N ; C=CC(=O)N1CCN(c2nc(NCCC(=O)NCc3ccc(-n4c(O)nnc4-c4ccc(O)cc4O)cc3)nc3c2CCN(c2cccc4cccc(Cl)c24)C3)C[C@@H]1CC#N ; CC(=O)N1CCN(c2nc(NCCC(=O)N3CCC(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cc(O)cc4ccccc24)C3)CC1] 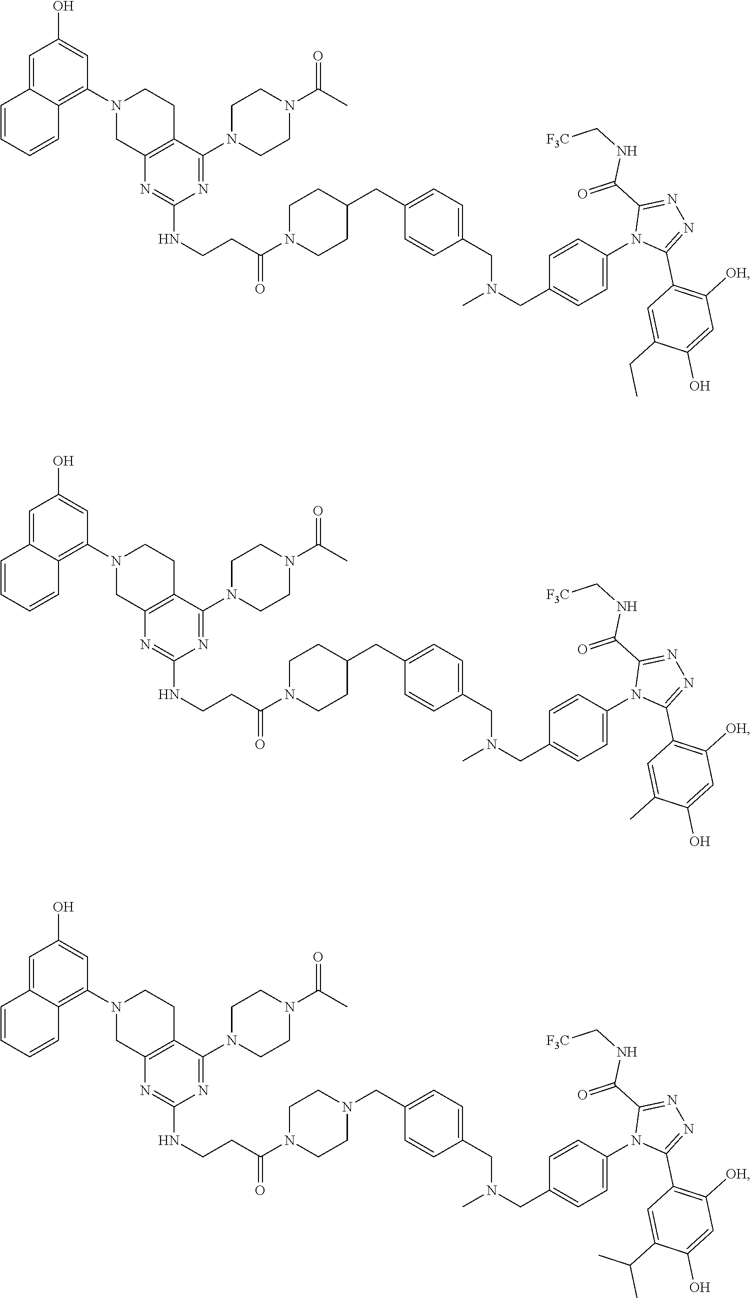 [SMILES: CCc1cc(-c2nnc(C(=O)NCC(F)(F)F)n2-c2ccc(CN(C)Cc3ccc(CC4CCN(C(=O)CCNc5nc6c(c(N7CCN(C(C)=O)CC7)n5)CCN(c5cc(O)cc7ccccc57)C6)CC4)cc3)cc2)c(O)cc1O ; CC(=O)N1CCN(c2nc(NCCC(=O)N3CCC(Cc4ccc(CN(C)Cc5ccc(-n6c(C(=O)NCC(F)(F)F)nnc6-c6cc(C)c(O)cc6O)cc5)cc4)CC3)nc3c2CCN(c2cc(O)cc4ccccc24)C3)CC1 ; CC(=O)N1CCN(c2nc(NCCC(=O)N3CCN(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cc(O)cc4ccccc24)C3)CC1] 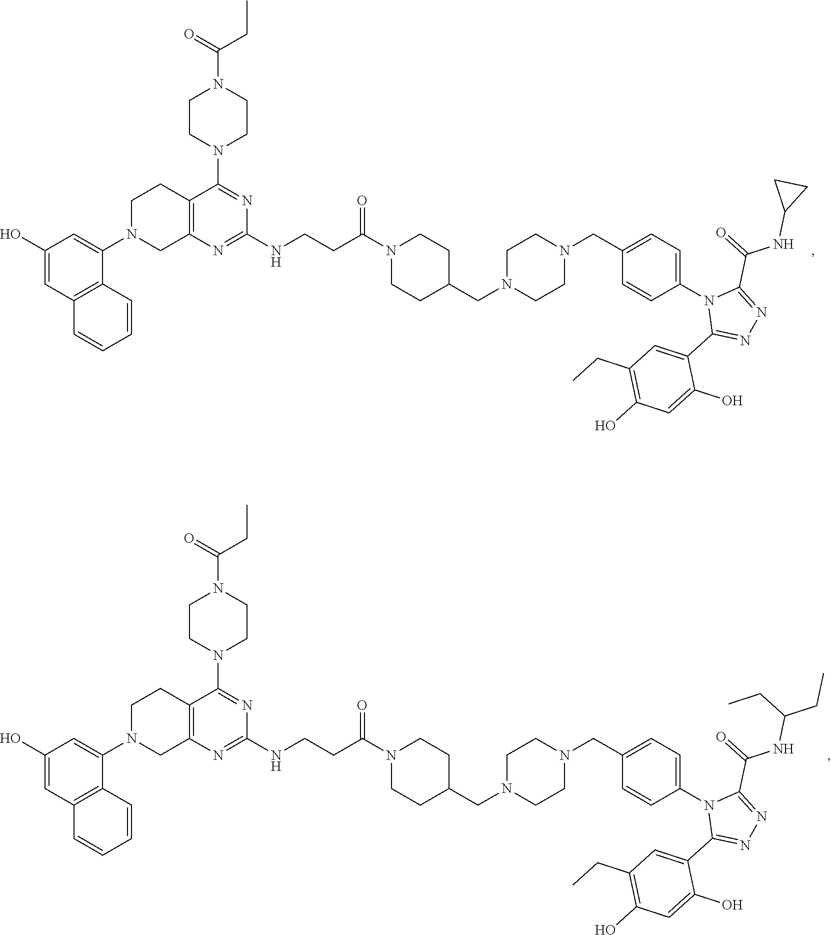 [SMILES: CCC(=O)N1CCN(c2nc(NCCC(=O)N3CCC(CN4CCN(Cc5ccc(-n6c(C(=O)NC7CC7)nnc6-c6cc(CC)c(O)cc6O)cc5)CC4)CC3)nc3c2CCN(c2cc(O)cc4ccccc24)C3)CC1 ; CCC(=O)N1CCN(c2nc(NCCC(=O)N3CCC(CN4CCN(Cc5ccc(-n6c(C(=O)NC(CC)CC)nnc6-c6cc(CC)c(O)cc6O)cc5)CC4)CC3)nc3c2CCN(c2cc(O)cc4ccccc24)C3)CC1] 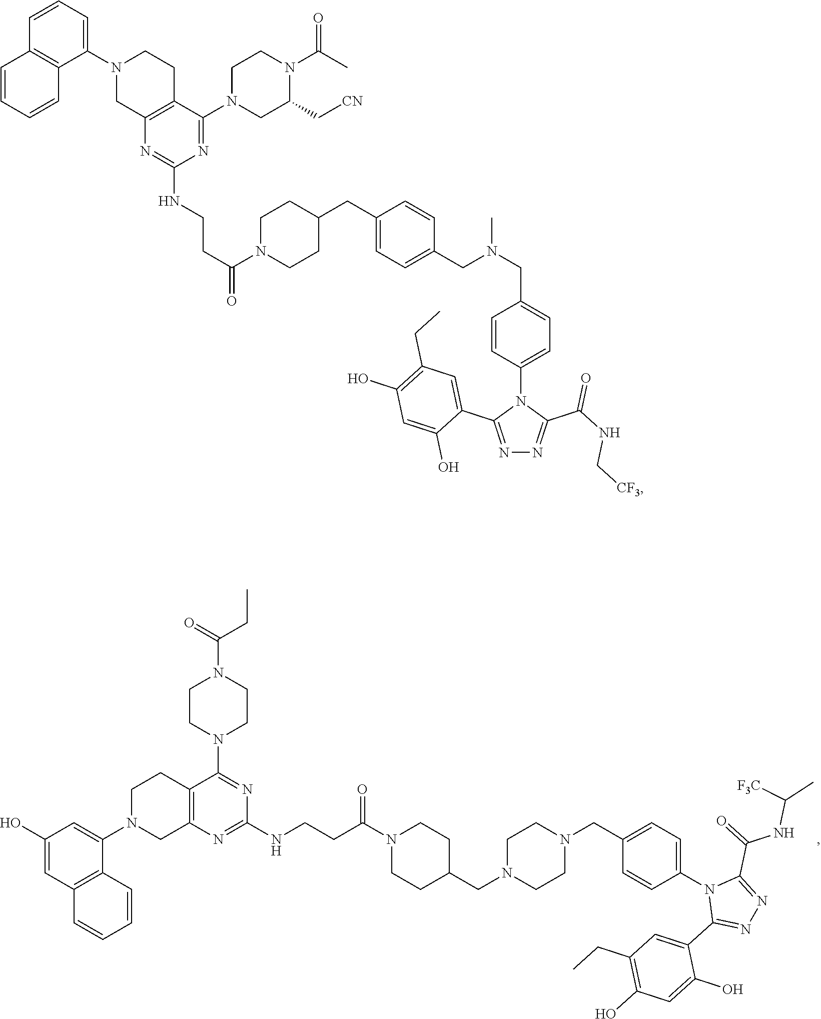 [SMILES: CCc1cc(-c2nnc(C(=O)NCC(F)(F)F)n2-c2ccc(CN(C)Cc3ccc(CC4CCN(C(=O)CCNc5nc6c(c(N7CCN(C(C)=O)[C@H](CC#N)C7)n5)CCN(c5cccc7ccccc57)C6)CC4)cc3)cc2)c(O)cc1O ; CCC(=O)N1CCN(c2nc(NCCC(=O)N3CCC(CN4CCN(Cc5ccc(-n6c(C(=O)NC(C)C(F)(F)F)nnc6-c6cc(CC)c(O)cc6O)cc5)CC4)CC3)nc3c2CCN(c2cc(O)cc4ccccc24)C3)CC1] 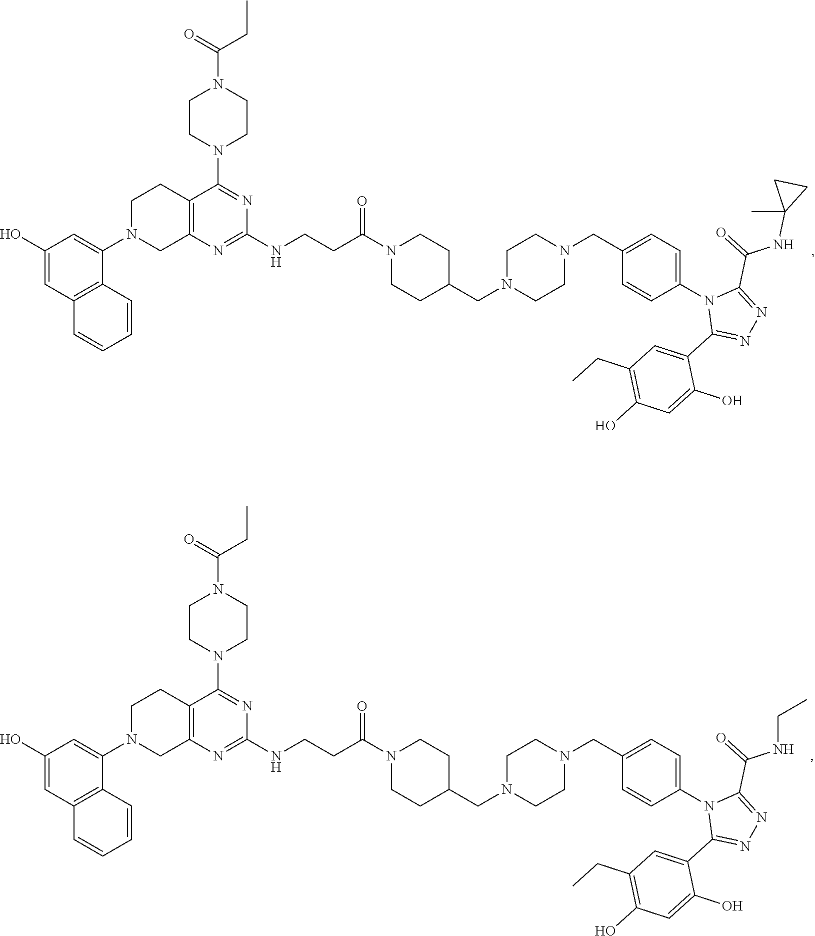 [SMILES: CCC(=O)N1CCN(c2nc(NCCC(=O)N3CCC(CN4CCN(Cc5ccc(-n6c(C(=O)NC7(C)CC7)nnc6-c6cc(CC)c(O)cc6O)cc5)CC4)CC3)nc3c2CCN(c2cc(O)cc4ccccc24)C3)CC1 ; CCNC(=O)c1nnc(-c2cc(CC)c(O)cc2O)n1-c1ccc(CN2CCN(CC3CCN(C(=O)CCNc4nc5c(c(N6CCN(C(=O)CC)CC6)n4)CCN(c4cc(O)cc6ccccc46)C5)CC3)CC2)cc1] 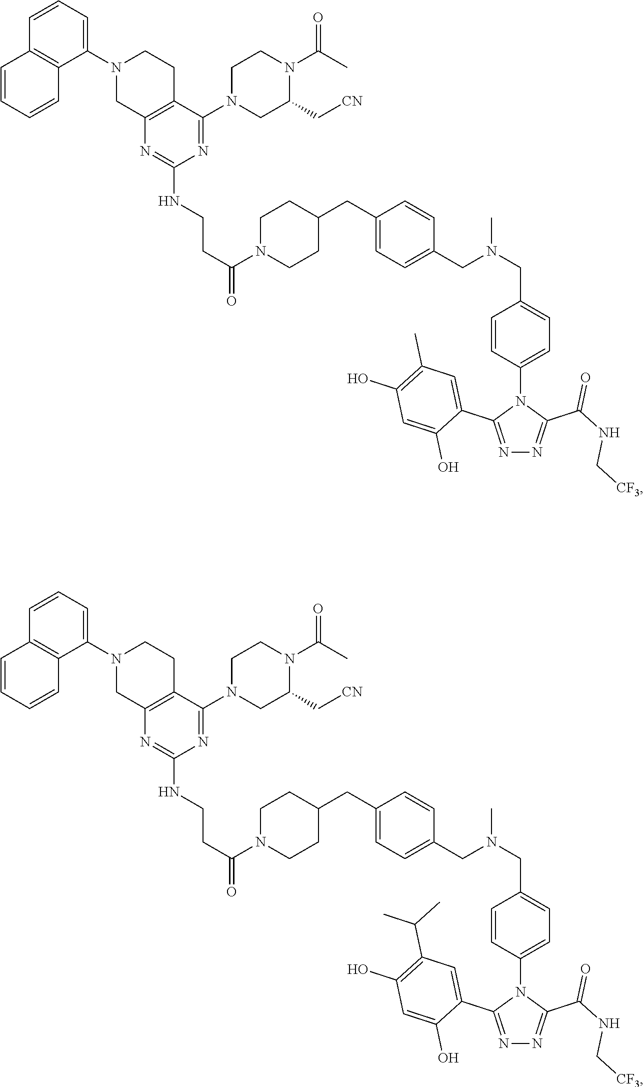 [SMILES: CC(=O)N1CCN(c2nc(NCCC(=O)N3CCC(Cc4ccc(CN(C)Cc5ccc(-n6c(C(=O)NCC(F)(F)F)nnc6-c6cc(C)c(O)cc6O)cc5)cc4)CC3)nc3c2CCN(c2cccc4ccccc24)C3)C[C@H]1CC#N ; CC(=O)N1CCN(c2nc(NCCC(=O)N3CCC(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cccc4ccccc24)C3)C[C@H]1CC#N] 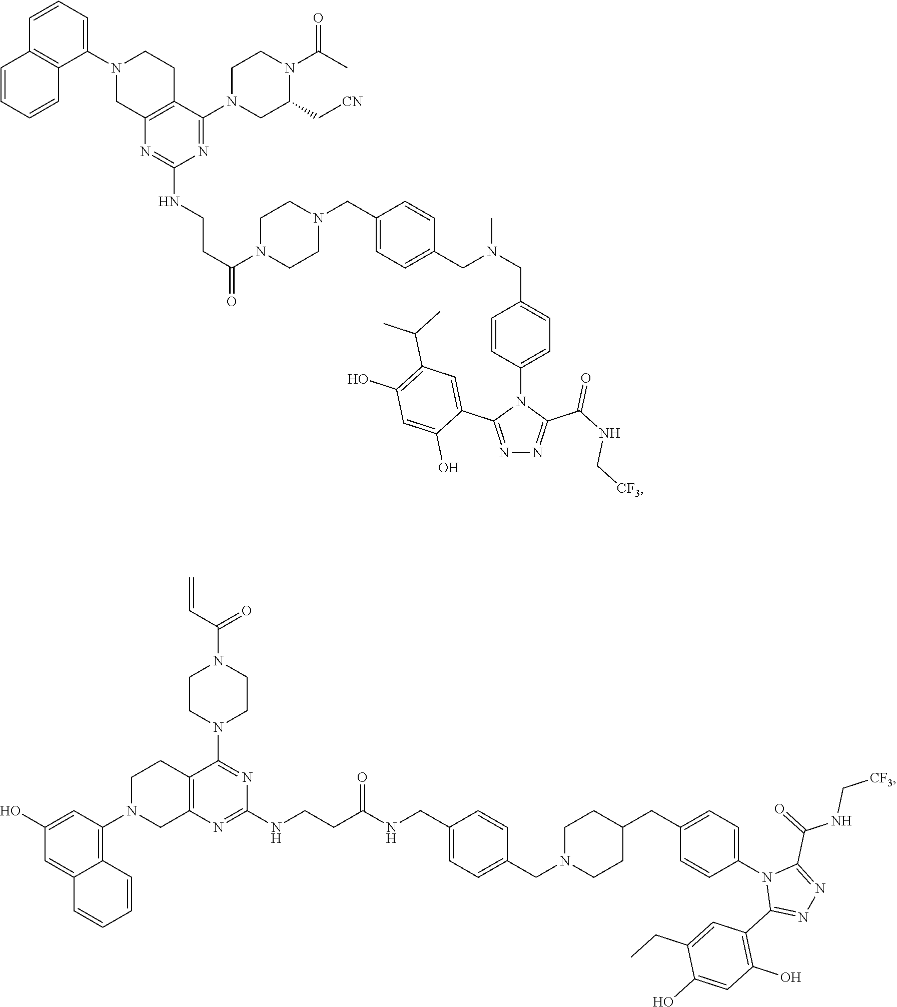 [SMILES: CC(=O)N1CCN(c2nc(NCCC(=O)N3CCN(Cc4ccc(CN(C)Cc5ccc(-n6c(C(=O)NCC(F)(F)F)nnc6-c6cc(C(C)C)c(O)cc6O)cc5)cc4)CC3)nc3c2CCN(c2cccc4ccccc24)C3)C[C@H]1CC#N ; C=CC(=O)N1CCN(c2nc(NCCC(=O)NCc3ccc(CN4CCC(Cc5ccc(-n6c(C(=O)NCC(F)(F)F)nnc6-c6cc(CC)c(O)cc6O)cc5)CC4)cc3)nc3c2CCN(c2cc(O)cc4ccccc24)C3)CC1] 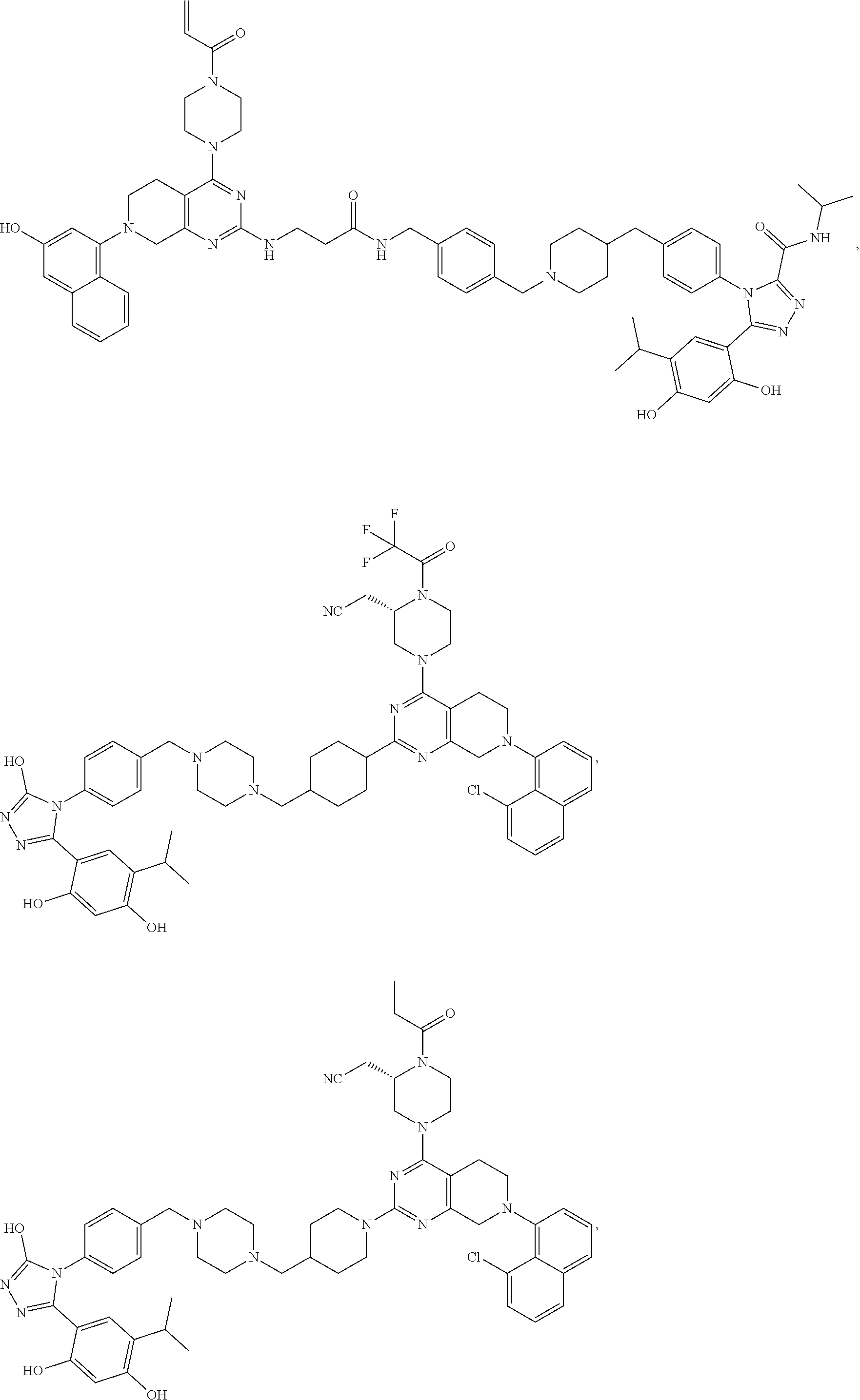 [SMILES: C=CC(=O)N1CCN(c2nc(NCCC(=O)NCc3ccc(CN4CCC(Cc5ccc(-n6c(C(=O)NC(C)C)nnc6-c6cc(C(C)C)c(O)cc6O)cc5)CC4)cc3)nc3c2CCN(c2cc(O)cc4ccccc24)C3)CC1 ; CC(C)c1cc(-c2nnc(O)n2-c2ccc(CN3CCN(CC4CCC(c5nc6c(c(N7CCN(C(=O)C(F)(F)F)[C@@H](CC#N)C7)n5)CCN(c5cccc7cccc(Cl)c57)C6)CC4)CC3)cc2)c(O)cc1O ; CCC(=O)N1CCN(c2nc(N3CCC(CN4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N] 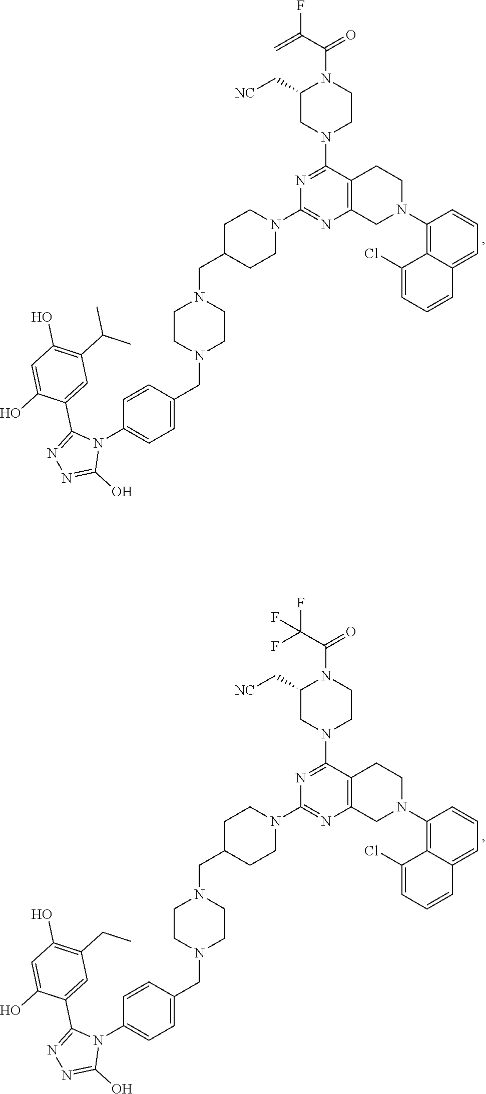 [SMILES: C=C(F)C(=O)N1CCN(c2nc(N3CCC(CN4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N ; CCc1cc(-c2nnc(O)n2-c2ccc(CN3CCN(CC4CCN(c5nc6c(c(N7CCN(C(=O)C(F)(F)F)[C@@H](CC#N)C7)n5)CCN(c5cccc7cccc(Cl)c57)C6)CC4)CC3)cc2)c(O)cc1O] 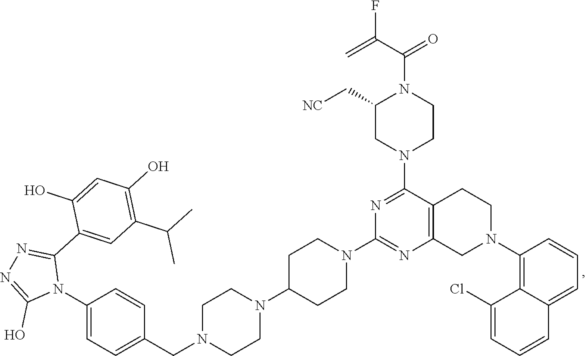 [SMILES: C=C(F)C(=O)N1CCN(c2nc(N3CCC(N4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N] 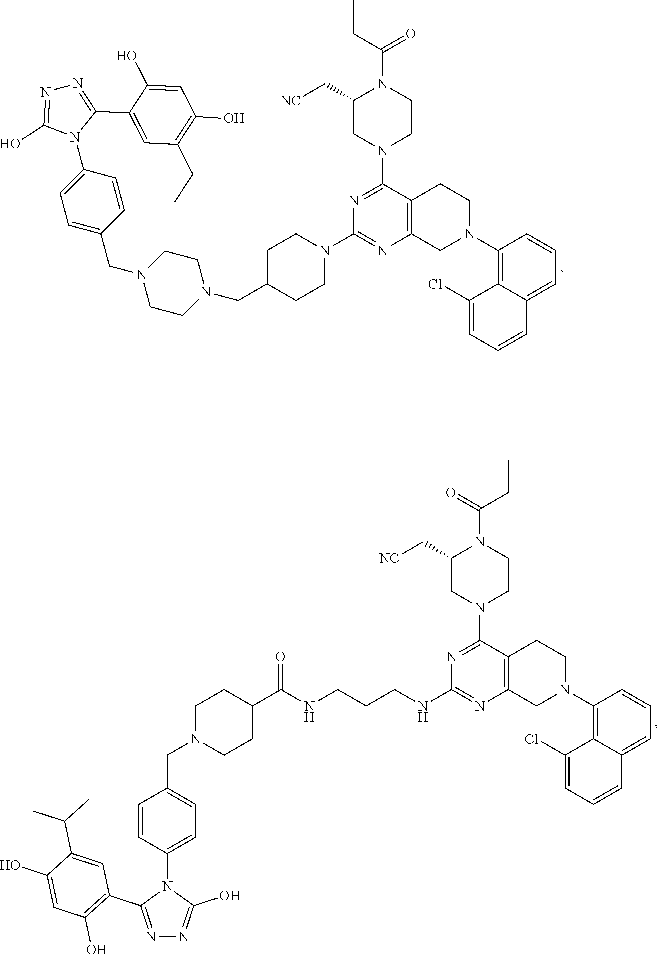 [SMILES: CCC(=O)N1CCN(c2nc(N3CCC(CN4CCN(Cc5ccc(-n6c(O)nnc6-c6cc(CC)c(O)cc6O)cc5)CC4)CC3)nc3c2CCN(c2cccc4cccc(Cl)c24)C3)C[C@@H]1CC#N ; CCC(=O)N1CCN(c2nc(NCCCNC(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N] 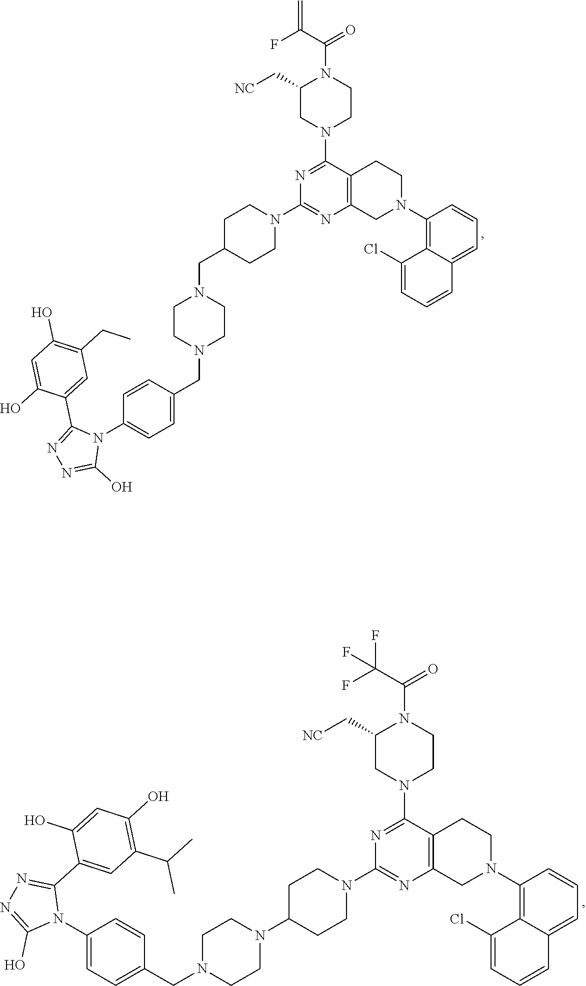 [SMILES: C=C(F)C(=O)N1CCN(c2nc(N3CCC(CN4CCN(Cc5ccc(-n6c(O)nnc6-c6cc(CC)c(O)cc6O)cc5)CC4)CC3)nc3c2CCN(c2cccc4cccc(Cl)c24)C3)C[C@@H]1CC#N ; CC(C)c1cc(-c2nnc(O)n2-c2ccc(CN3CCN(C4CCN(c5nc6c(c(N7CCN(C(=O)C(F)(F)F)[C@@H](CC#N)C7)n5)CCN(c5cccc7cccc(Cl)c57)C6)CC4)CC3)cc2)c(O)cc1O] 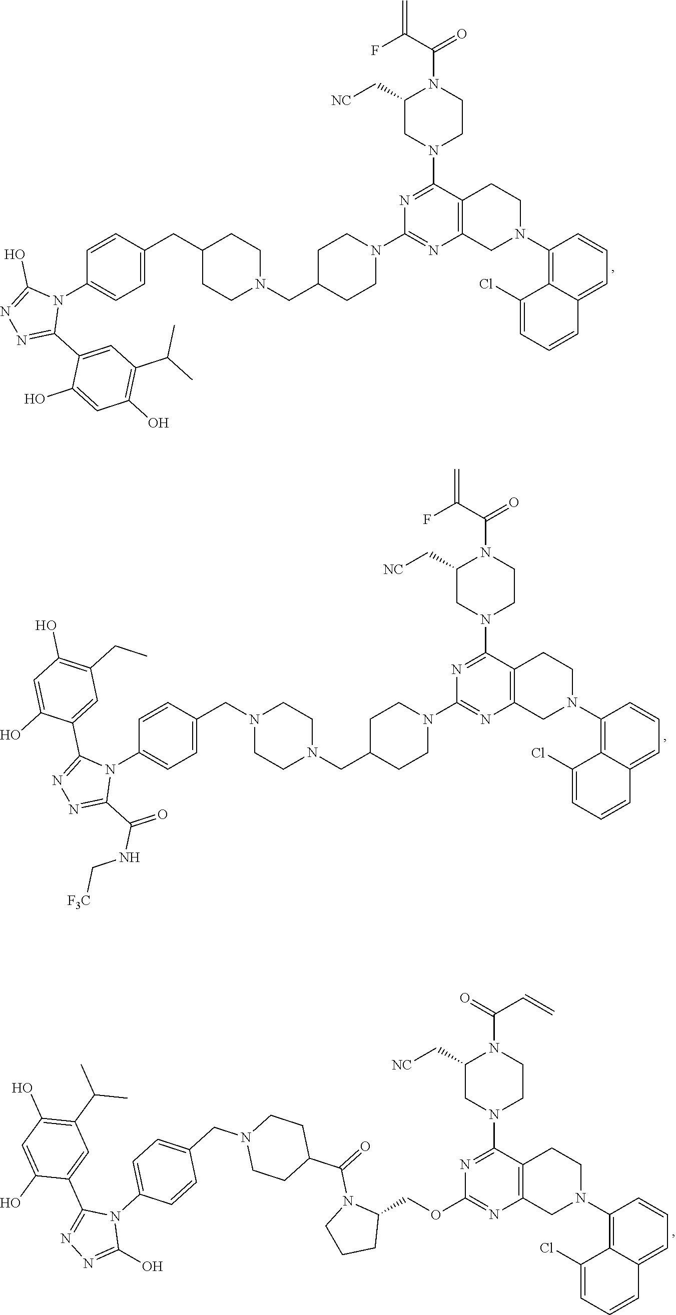 [SMILES: C=C(F)C(=O)N1CCN(c2nc(N3CCC(CN4CCC(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N ; C=C(F)C(=O)N1CCN(c2nc(N3CCC(CN4CCN(Cc5ccc(-n6c(C(=O)NCC(F)(F)F)nnc6-c6cc(CC)c(O)cc6O)cc5)CC4)CC3)nc3c2CCN(c2cccc4cccc(Cl)c24)C3)C[C@@H]1CC#N ; C=CC(=O)N1CCN(c2nc(OC[C@@H]3CCCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N] 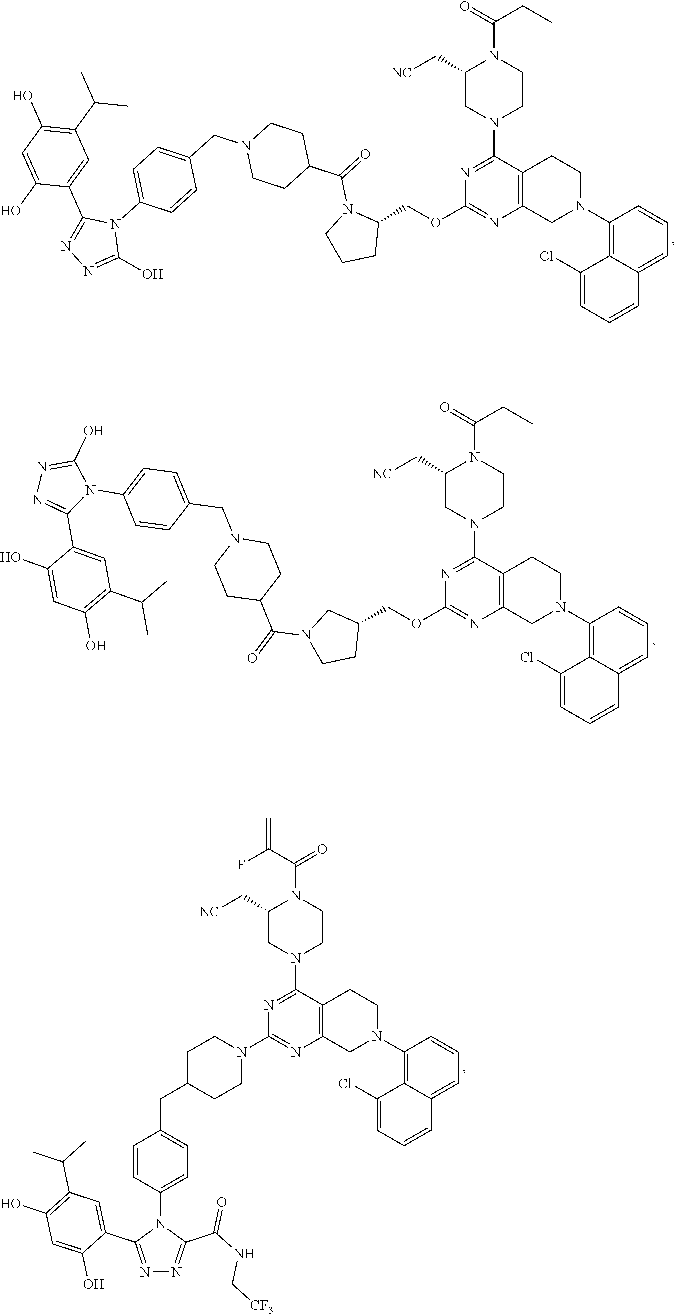 [SMILES: CCC(=O)N1CCN(c2nc(OC[C@@H]3CCCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N ; CCC(=O)N1CCN(c2nc(OC[C@@H]3CCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N ; C=C(F)C(=O)N1CCN(c2nc(N3CCC(Cc4ccc(-n5c(C(=O)NCC(F)(F)F)nnc5-c5cc(C(C)C)c(O)cc5O)cc4)CC3)nc3c2CCN(c2cccc4cccc(Cl)c24)C3)C[C@@H]1CC#N] 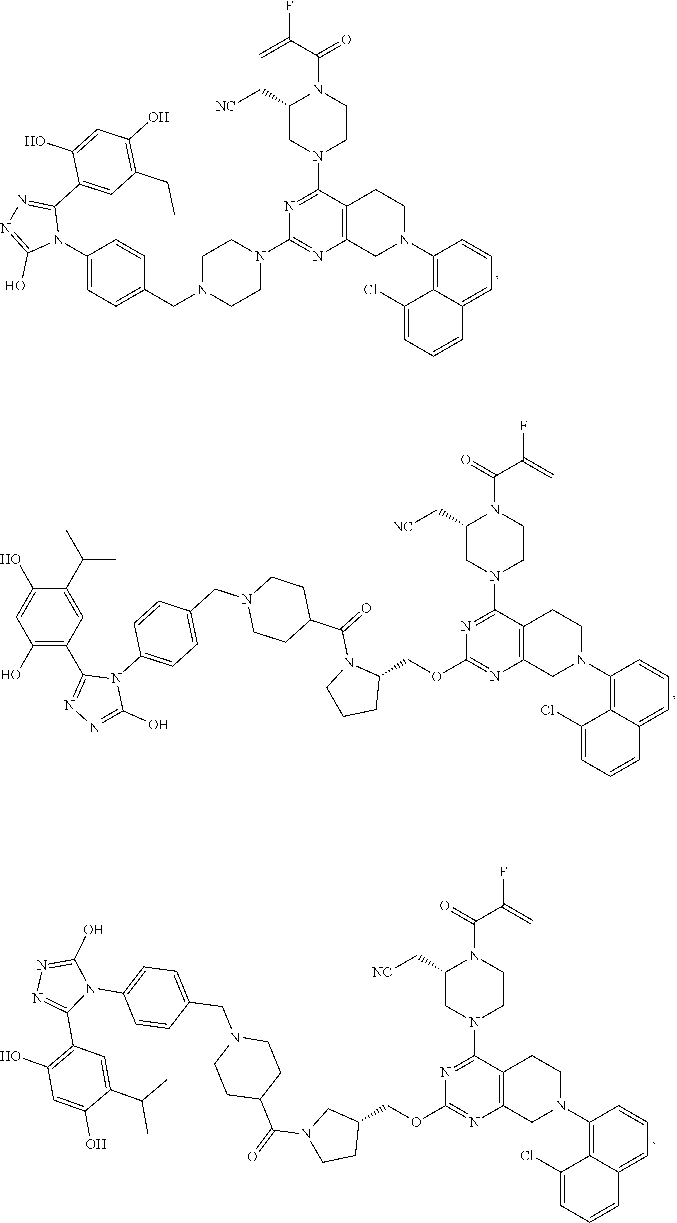 [SMILES: C=C(F)C(=O)N1CCN(c2nc(N3CCN(Cc4ccc(-n5c(O)nnc5-c5cc(CC)c(O)cc5O)cc4)CC3)nc3c2CCN(c2cccc4cccc(Cl)c24)C3)C[C@@H]1CC#N ; C=C(F)C(=O)N1CCN(c2nc(OC[C@@H]3CCCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N ; C=C(F)C(=O)N1CCN(c2nc(OC[C@@H]3CCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N] 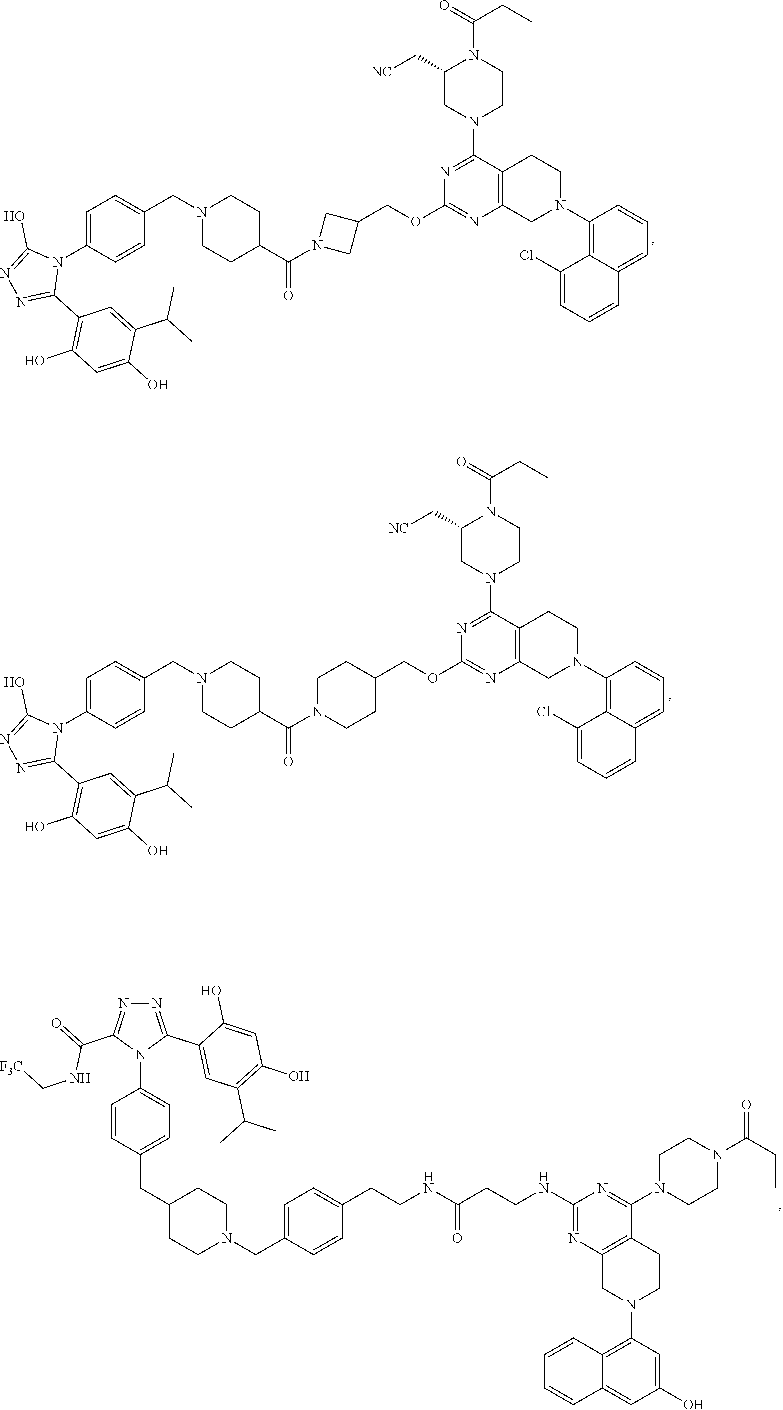 [SMILES: CCC(=O)N1CCN(c2nc(OCC3CN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N ; CCC(=O)N1CCN(c2nc(OCC3CCN(C(=O)C4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N ; CCC(=O)N1CCN(c2nc(NCCC(=O)NCCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1] 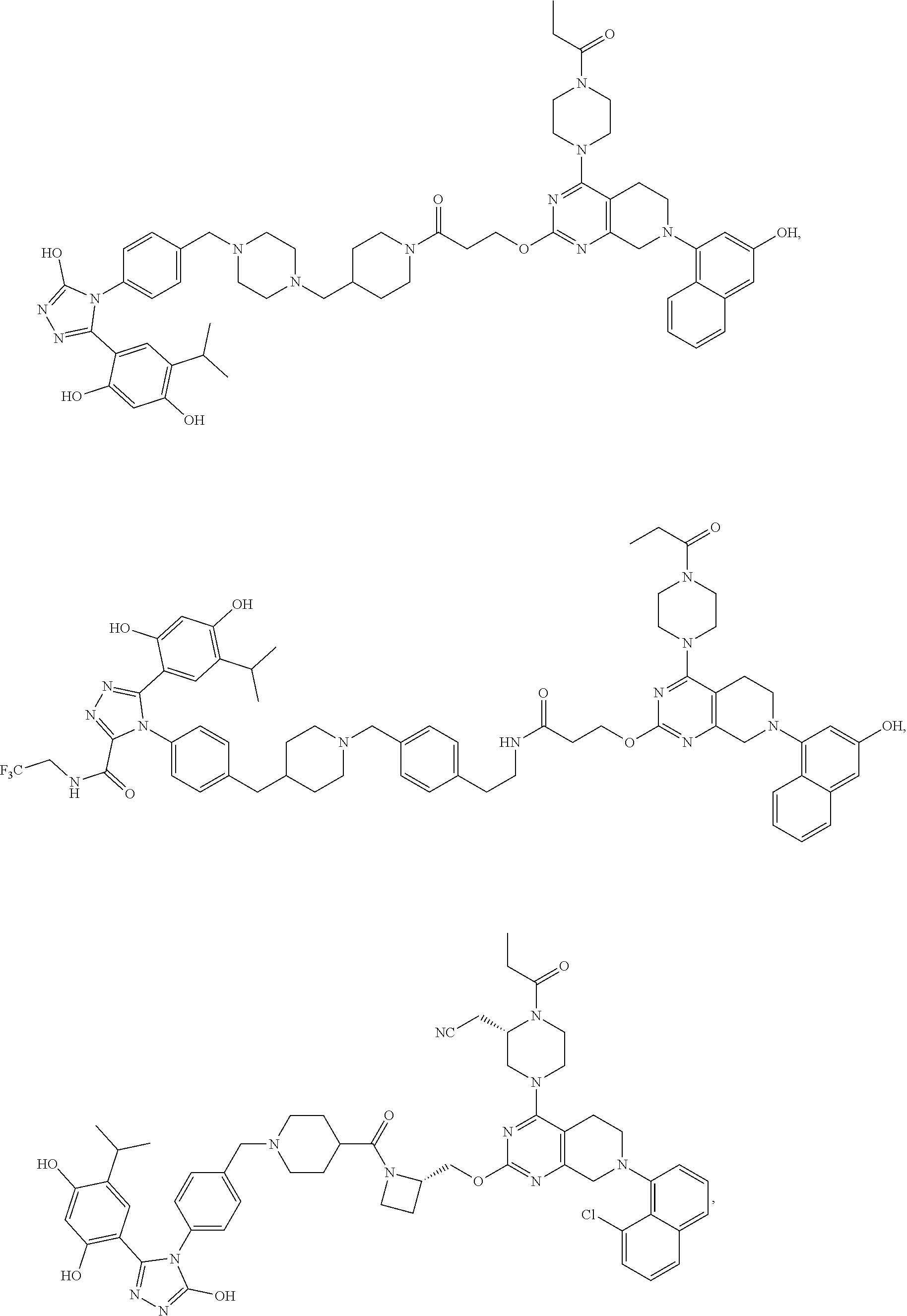 [SMILES: CCC(=O)N1CCN(c2nc(OCCC(=O)N3CCC(CN4CCN(Cc5ccc(-n6c(O)nnc6-c6cc(C(C)C)c(O)cc6O)cc5)CC4)CC3)nc3c2CCN(c2cc(O)cc4ccccc24)C3)CC1 ; CCC(=O)N1CCN(c2nc(OCCC(=O)NCCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; CCC(=O)N1CCN(c2nc(OC[C@@H]3CCN3C(=O)C3CCN(Cc4ccc(-n5c(O)nnc5-c5cc(C(C)C)c(O)cc5O)cc4)CC3)nc3c2CCN(c2cccc4cccc(Cl)c24)C3)C[C@@H]1CC#N] 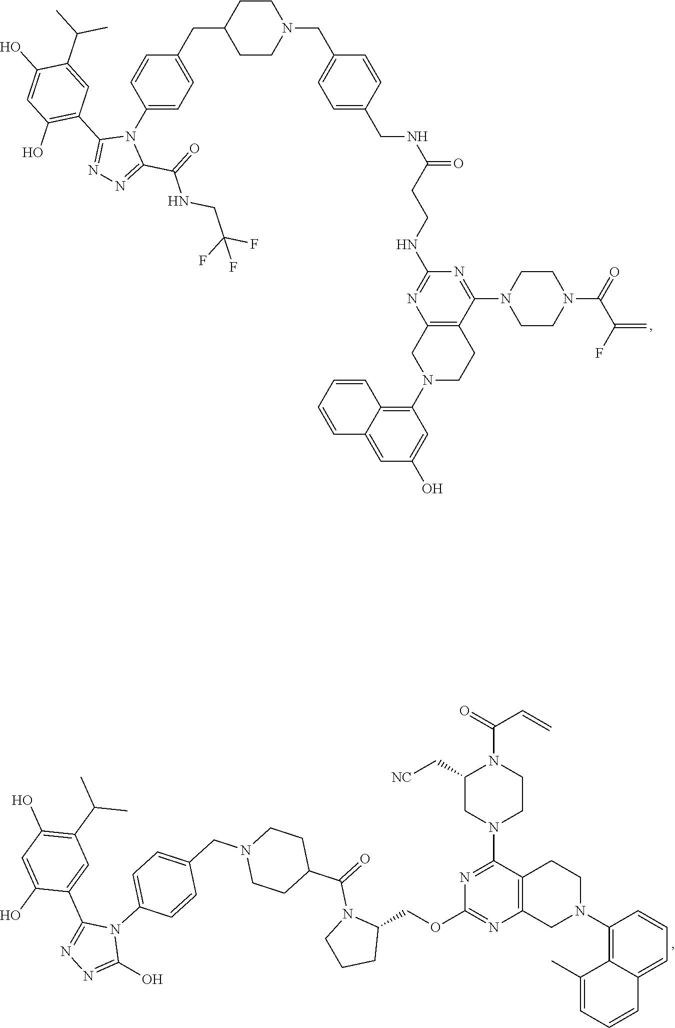 [SMILES: C=C(F)C(=O)N1CCN(c2nc(NCCC(=O)NCc3ccc(CN4CCC(Cc5ccc(-n6c(C(=O)NCC(F)(F)F)nnc6-c6cc(C(C)C)c(O)cc6O)cc5)CC4)cc3)nc3c2CCN(c2cc(O)cc4ccccc24)C3)CC1 ; C=CC(=O)N1CCN(c2nc(OC[C@@H]3CCCN3C(=O)C3CCN(Cc4ccc(-n5c(O)nnc5-c5cc(C(C)C)c(O)cc5O)cc4)CC3)nc3c2CCN(c2cccc4cccc(C)c24)C3)C[C@@H]1CC#N] 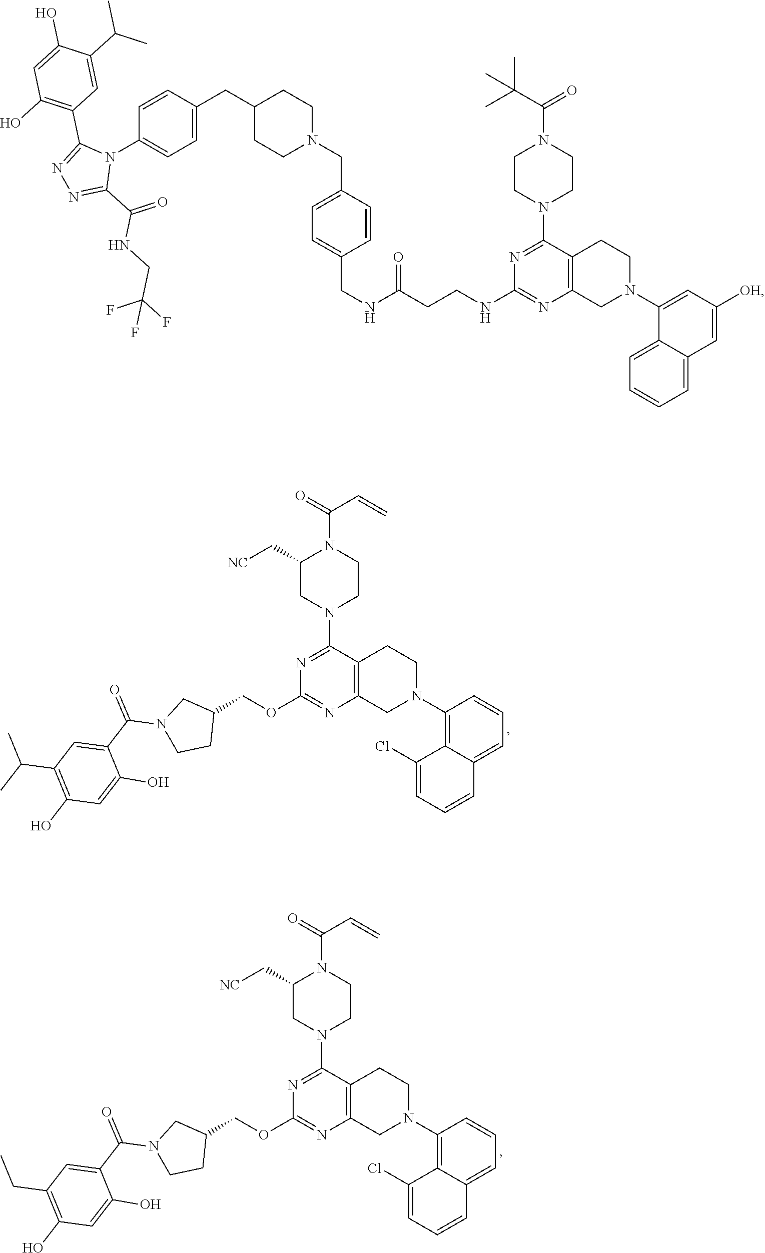 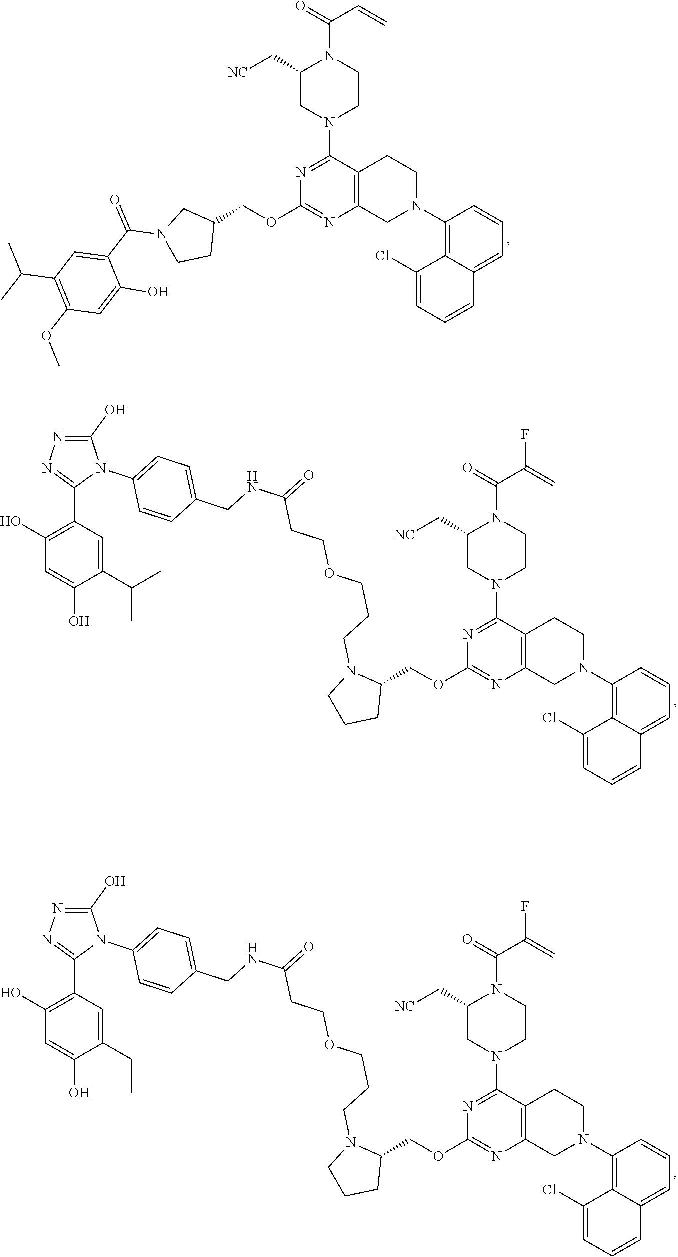 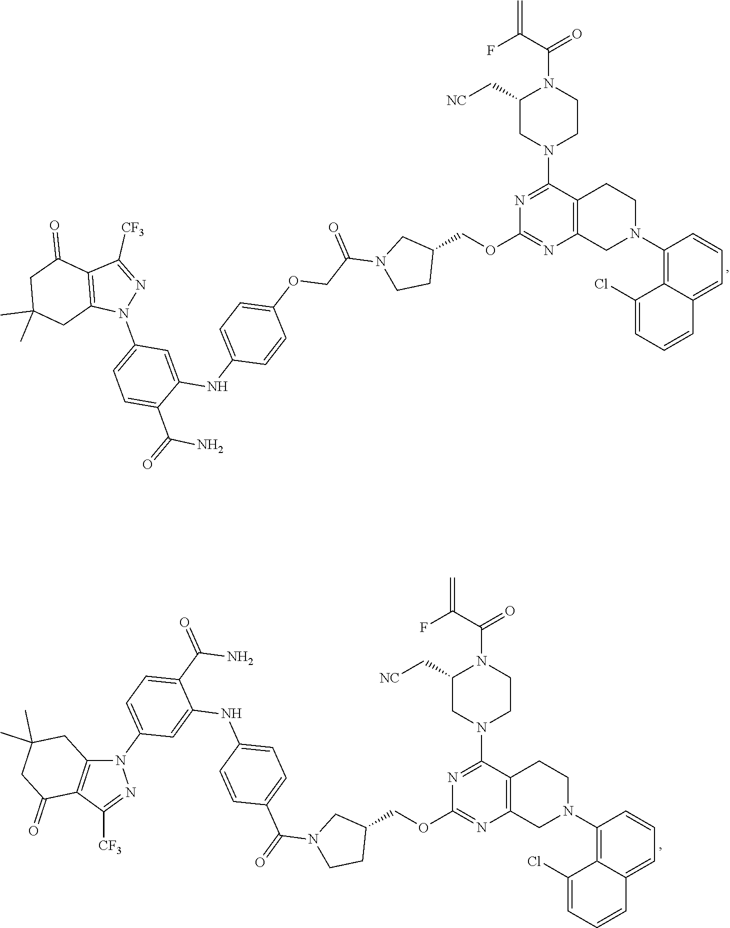 [SMILES: C=C(F)C(=O)N1CCN(c2nc(OC[C@@H]3CCN(C(=O)COc4ccc(Nc5cc(-n6nc(C(F)(F)F)c7c6CC(C)(C)CC7=O)ccc5C(N)=O)cc4)C3)nc3c2CCN(c2cccc4cccc(Cl)c24)C3)C[C@@H]1CC#N ; C=C(F)C(=O)N1CCN(c2nc(OC[C@@H]3CCN(C(=O)c4ccc(Nc5cc(-n6nc(C(F)(F)F)c7c6CC(C)(C)CC7=O)ccc5C(N)=O)cc4)C3)nc3c2CCN(c2cccc4cccc(Cl)c24)C3)C[C@@H]1CC#N] 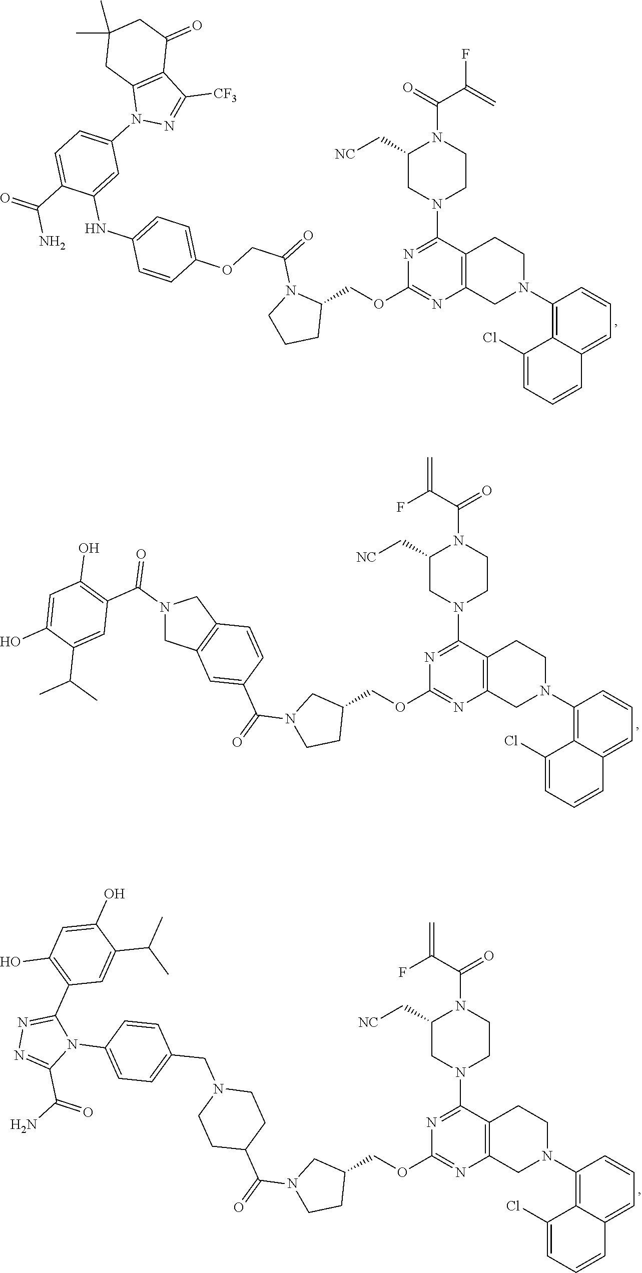 [SMILES: C=C(F)C(=O)N1CCN(c2nc(OC[C@@H]3CCCN3C(=O)COc3ccc(Nc4cc(-n5nc(C(F)(F)F)c6c5CC(C)(C)CC6=O)ccc4C(N)=O)cc3)nc3c2CCN(c2cccc4cccc(Cl)c24)C3)C[C@@H]1CC#N ; C=C(F)C(=O)N1CCN(c2nc(OC[C@@H]3CCN(C(=O)c4ccc5c(c4)CN(C(=O)c4cc(C(C)C)c(O)cc4O)C5)C3)nc3c2CCN(c2cccc4cccc(Cl)c24)C3)C[C@@H]1CC#N ; C=C(F)C(=O)N1CCN(c2nc(OC[C@@H]3CCN(C(=O)C4CCN(Cc5ccc(-n6c(C(N)=O)nnc6-c6cc(C(C)C)c(O)cc6O)cc5)CC4)C3)nc3c2CCN(c2cccc4cccc(Cl)c24)C3)C[C@@H]1CC#N] 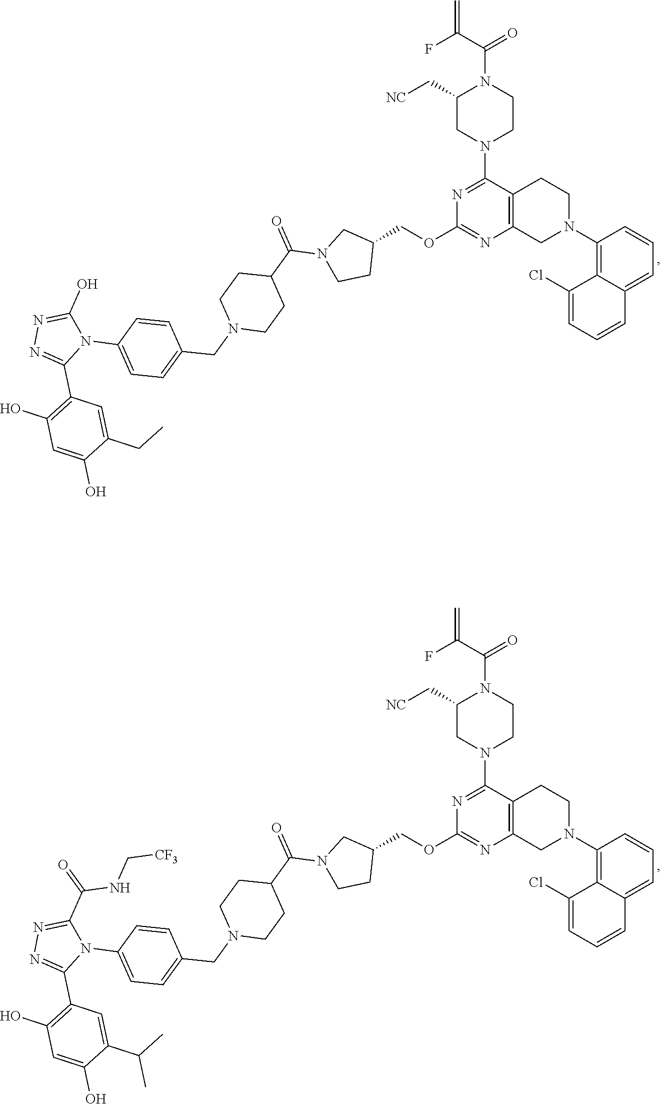 [SMILES: C=C(F)C(=O)N1CCN(c2nc(OC[C@@H]3CCN(C(=O)C4CCN(Cc5ccc(-n6c(O)nnc6-c6cc(CC)c(O)cc6O)cc5)CC4)C3)nc3c2CCN(c2cccc4cccc(Cl)c24)C3)C[C@@H]1CC#N ; C=C(F)C(=O)N1CCN(c2nc(OC[C@@H]3CCN(C(=O)C4CCN(Cc5ccc(-n6c(C(=O)NCC(F)(F)F)nnc6-c6cc(C(C)C)c(O)cc6O)cc5)CC4)C3)nc3c2CCN(c2cccc4cccc(Cl)c24)C3)C[C@@H]1CC#N] 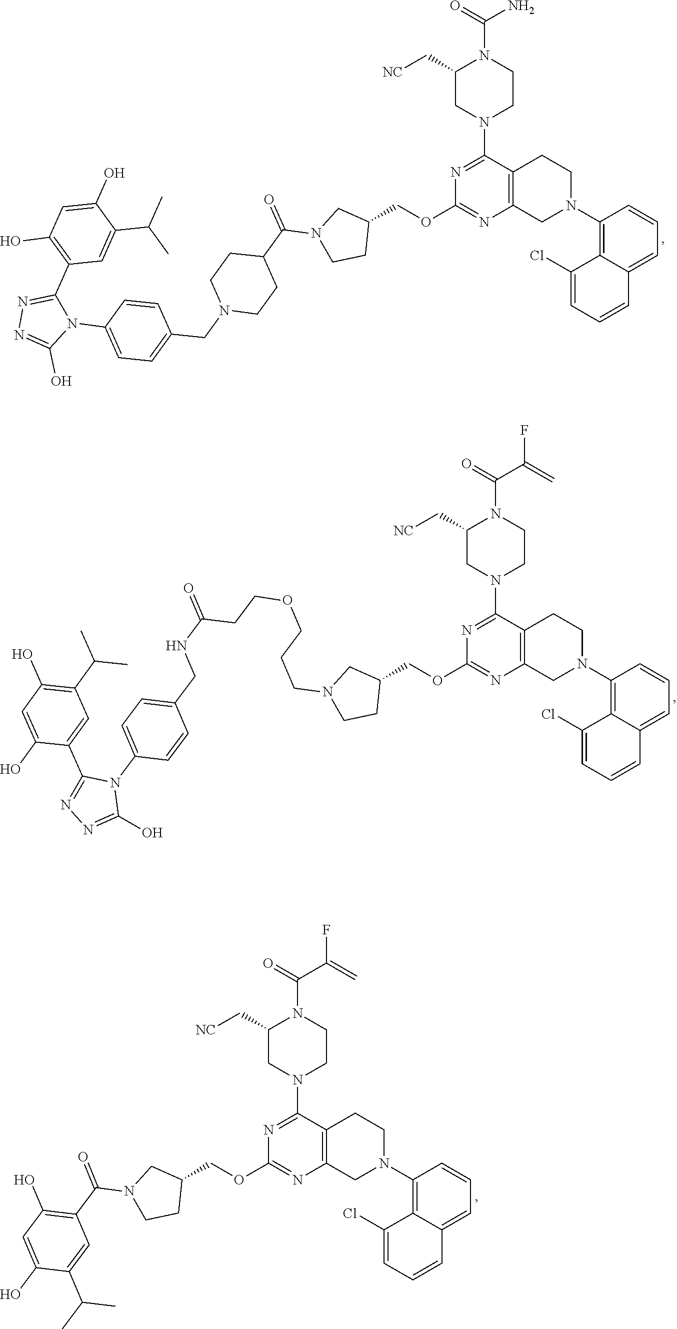 [SMILES: CC(C)c1cc(-c2nnc(O)n2-c2ccc(CN3CCC(C(=O)N4CC[C@@H](COc5nc6c(c(N7CCN(C(N)=O)[C@@H](CC#N)C7)n5)CCN(c5cccc7cccc(Cl)c57)C6)C4)CC3)cc2)c(O)cc1O ; C=C(F)C(=O)N1CCN(c2nc(OC[C@@H]3CCN(CCCOCCC(=O)NCc4ccc(-n5c(O)nnc5-c5cc(C(C)C)c(O)cc5O)cc4)C3)nc3c2CCN(c2cccc4cccc(Cl)c24)C3)C[C@@H]1CC#N ; C=C(F)C(=O)N1CCN(c2nc(OC[C@@H]3CCN(C(=O)c4cc(C(C)C)c(O)cc4O)C3)nc3c2CCN(c2cccc4cccc(Cl)c24)C3)C[C@@H]1CC#N] 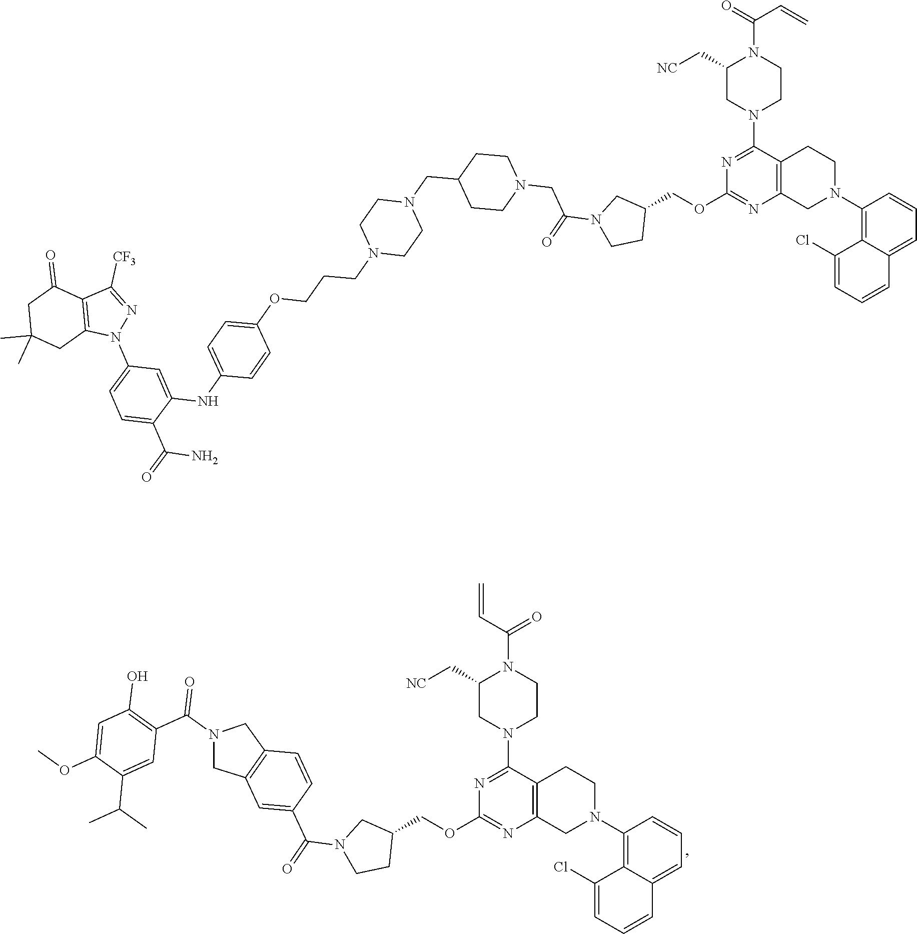 [SMILES: C=CC(=O)N1CCN(c2nc(OC[C@@H]3CCN(C(=O)CN4CCC(CN5CCN(CCCOc6ccc(Nc7cc(-n8nc(C(F)(F)F)c9c8CC(C)(C)CC9=O)ccc7C(N)=O)cc6)CC5)CC4)C3)nc3c2CCN(c2cccc4cccc(Cl)c24)C3)C[C@@H]1CC#N ; C=CC(=O)N1CCN(c2nc(OC[C@@H]3CCN(C(=O)c4ccc5c(c4)CN(C(=O)c4cc(C(C)C)c(OC)cc4O)C5)C3)nc3c2CCN(c2cccc4cccc(Cl)c24)C3)C[C@@H]1CC#N] 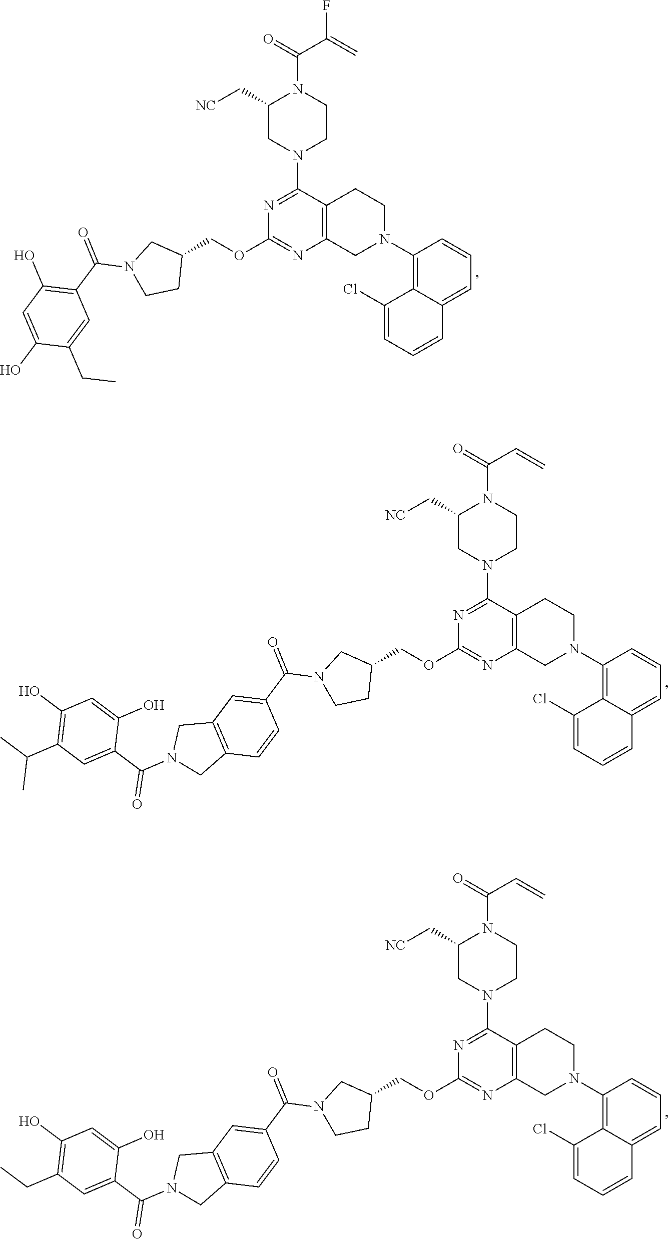 [SMILES: C=C(F)C(=O)N1CCN(c2nc(OC[C@@H]3CCN(C(=O)c4cc(CC)c(O)cc4O)C3)nc3c2CCN(c2cccc4cccc(Cl)c24)C3)C[C@@H]1CC#N ; C=CC(=O)N1CCN(c2nc(OC[C@@H]3CCN(C(=O)c4ccc5c(c4)CN(C(=O)c4cc(C(C)C)c(O)cc4O)C5)C3)nc3c2CCN(c2cccc4cccc(Cl)c24)C3)C[C@@H]1CC#N ; C=CC(=O)N1CCN(c2nc(OC[C@@H]3CCN(C(=O)c4ccc5c(c4)CN(C(=O)c4cc(CC)c(O)cc4O)C5)C3)nc3c2CCN(c2cccc4cccc(Cl)c24)C3)C[C@@H]1CC#N] 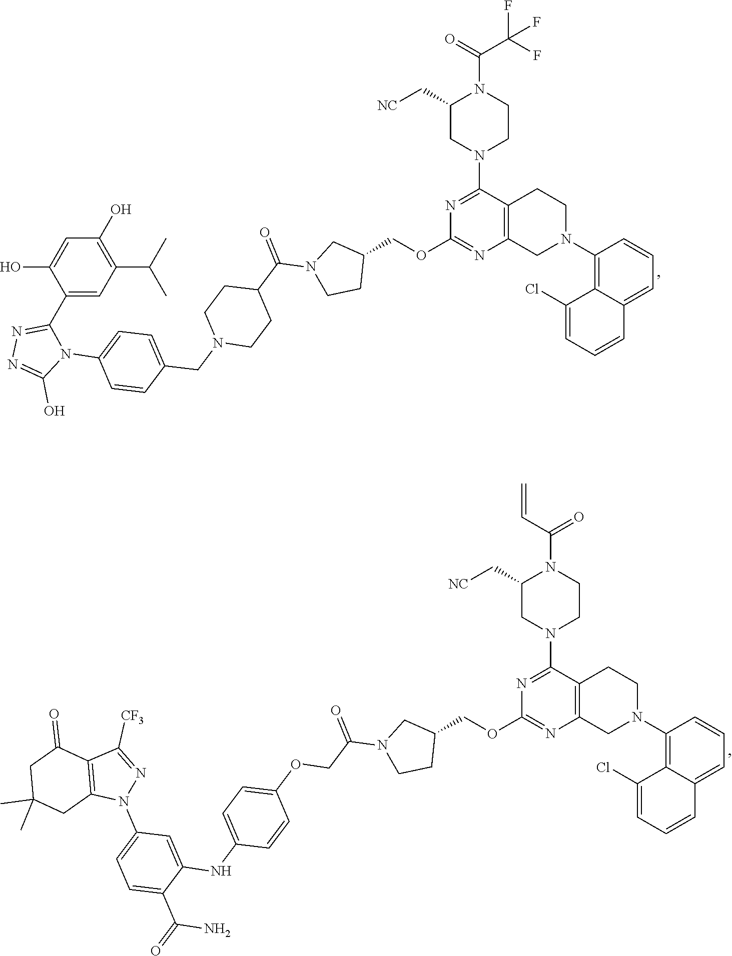 [SMILES: CC(C)c1cc(-c2nnc(O)n2-c2ccc(CN3CCC(C(=O)N4CC[C@@H](COc5nc6c(c(N7CCN(C(=O)C(F)(F)F)[C@@H](CC#N)C7)n5)CCN(c5cccc7cccc(Cl)c57)C6)C4)CC3)cc2)c(O)cc1O ; C=CC(=O)N1CCN(c2nc(OC[C@@H]3CCN(C(=O)COc4ccc(Nc5cc(-n6nc(C(F)(F)F)c7c6CC(C)(C)CC7=O)ccc5C(N)=O)cc4)C3)nc3c2CCN(c2cccc4cccc(Cl)c24)C3)C[C@@H]1CC#N] 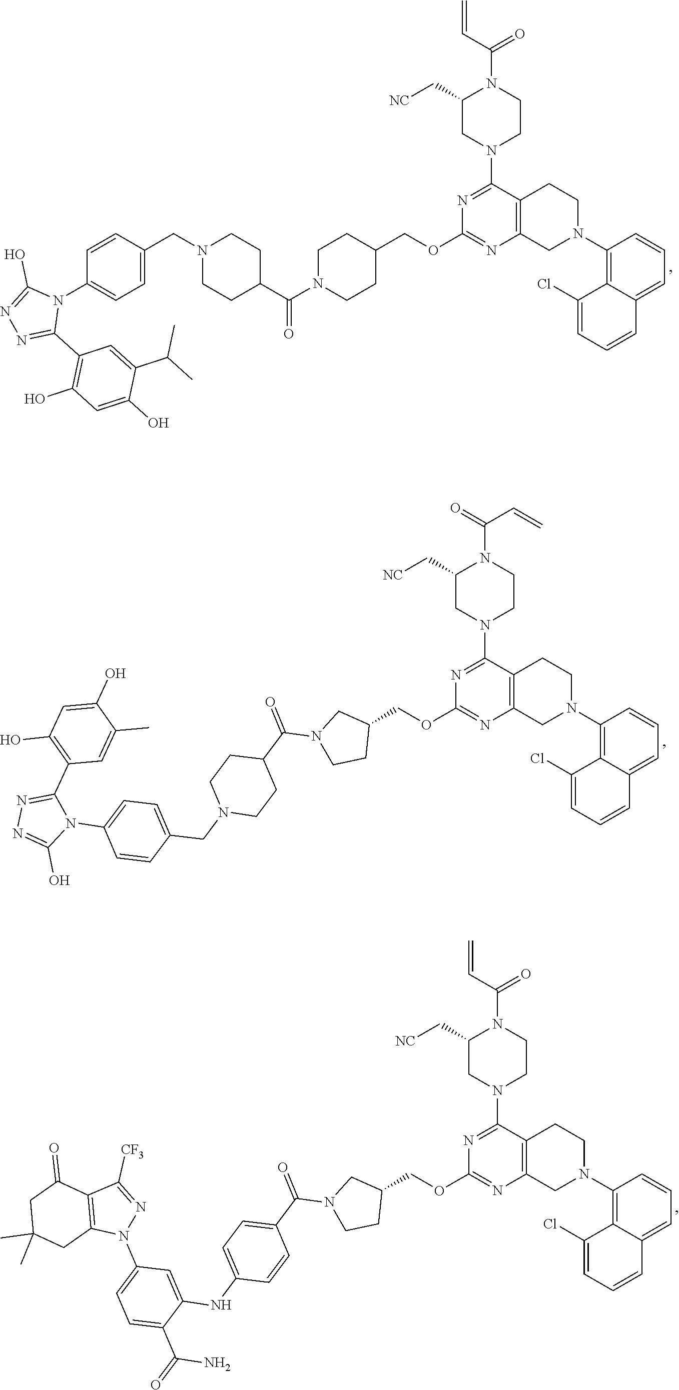 [SMILES: C=CC(=O)N1CCN(c2nc(OCC3CCN(C(=O)C4CCN(Cc5ccc(-n6c(O)nnc6-c6cc(C(C)C)c(O)cc6O)cc5)CC4)CC3)nc3c2CCN(c2cccc4cccc(Cl)c24)C3)C[C@@H]1CC#N ; C=CC(=O)N1CCN(c2nc(OC[C@@H]3CCN(C(=O)C4CCN(Cc5ccc(-n6c(O)nnc6-c6cc(C)c(O)cc6O)cc5)CC4)C3)nc3c2CCN(c2cccc4cccc(Cl)c24)C3)C[C@@H]1CC#N ; C=CC(=O)N1CCN(c2nc(OC[C@@H]3CCN(C(=O)c4ccc(Nc5cc(-n6nc(C(F)(F)F)c7c6CC(C)(C)CC7=O)ccc5C(N)=O)cc4)C3)nc3c2CCN(c2cccc4cccc(Cl)c24)C3)C[C@@H]1CC#N] 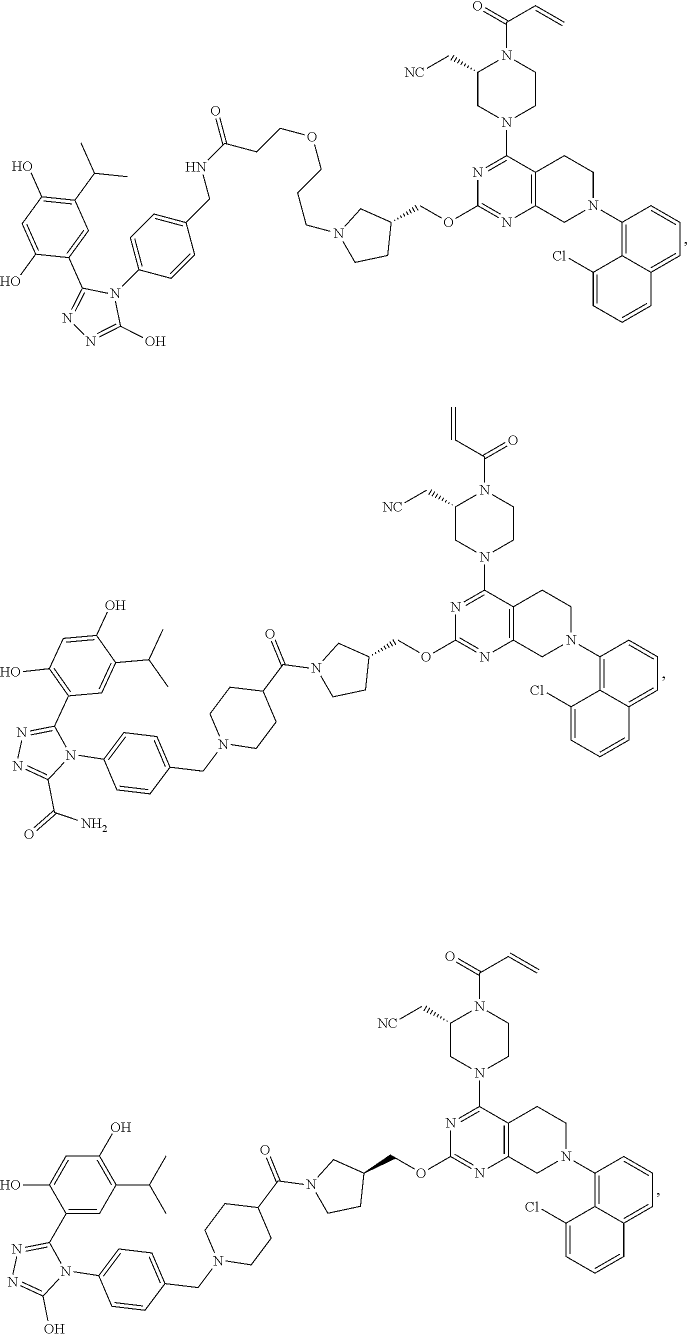 [SMILES: C=CC(=O)N1CCN(c2nc(OC[C@@H]3CCN(CCCOCCC(=O)NCc4ccc(-n5c(O)nnc5-c5cc(C(C)C)c(O)cc5O)cc4)C3)nc3c2CCN(c2cccc4cccc(Cl)c24)C3)C[C@@H]1CC#N ; C=CC(=O)N1CCN(c2nc(OC[C@@H]3CCN(C(=O)C4CCN(Cc5ccc(-n6c(C(N)=O)nnc6-c6cc(C(C)C)c(O)cc6O)cc5)CC4)C3)nc3c2CCN(c2cccc4cccc(Cl)c24)C3)C[C@@H]1CC#N ; C=CC(=O)N1CCN(c2nc(OC[C@H]3CCN(C(=O)C4CCN(Cc5ccc(-n6c(O)nnc6-c6cc(C(C)C)c(O)cc6O)cc5)CC4)C3)nc3c2CCN(c2cccc4cccc(Cl)c24)C3)C[C@@H]1CC#N] 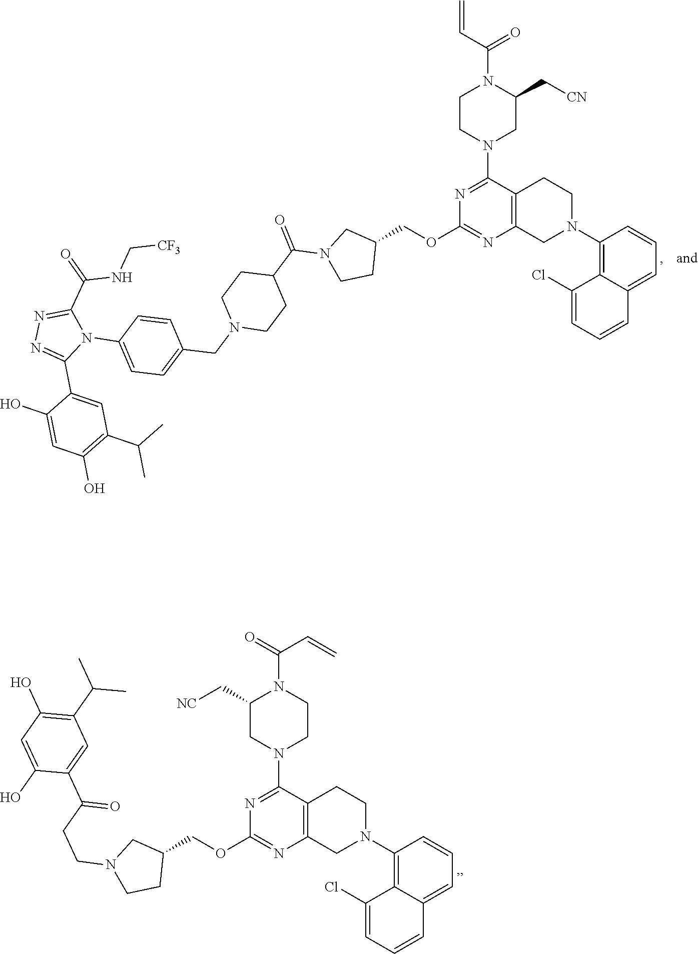 [SMILES: C=CC(=O)N1CCN(c2nc(OC[C@@H]3CCN(C(=O)C4CCN(Cc5ccc(-n6c(C(=O)NCC(F)(F)F)nnc6-c6cc(C(C)C)c(O)cc6O)cc5)CC4)C3)nc3c2CCN(c2cccc4cccc(Cl)c24)C3)C[C@@H]1CC#N ; C=CC(=O)N1CCN(c2nc(OC[C@@H]3CCN(CCC(=O)c4cc(C(C)C)c(O)cc4O)C3)nc3c2CCN(c2cccc4cccc(Cl)c24)C3)C[C@@H]1CC#N]
or a pharmaceutically acceptable salt of any of the foregoing.

41. The compound of claim 1, wherein the compound is selected from compounds 187 to 351, or a pharmaceutically acceptable salt of any of compounds 187 to 351.

42. A pharmaceutical composition comprising the compound of claim 1, or a pharmaceutically acceptable salt thereof, and a pharmaceutically acceptable carrier.

43. A method of treating cancer comprising administering to a subject a therapeutically effective amount of a compound of claim 1, or a pharmaceutically acceptable salt thereof.
